(19) United States
(12) Patent Application Publication
(10) Pub. No.: US 2025/0243248 A1
(43) Pub. Date: Jul. 31, 2025

(54) REPROGRAMMABLE TNPB POLYPEPTIDES WITH MAZE DOMAINS AND USES THEREOF

(71) Applicants: The Broad Institute, Inc., Cambridge, MA (US); Massachusetts Institute of Technology, Cambridge, MA (US)

(72) Inventors: Feng Zhang, Cambridge, MA (US); Han Altae-Tran, Cambridge, MA (US); Soumya Kannan, Cambridge, MA (US)

(21) Appl. No.: 19/176,309

(22) Filed: Apr. 11, 2025

Related U.S. Application Data
(63) Continuation of application No. PCT/US2023/076591, filed on Oct. 11, 2023.
(60) Provisional application No. 63/415,172, filed on Oct. 11, 2022.

(51) Int. Cl. C07K 14/245; C12N 9/12; C12N 9/78; C12N 15/90; C12Q 1/6823; C12Q 1/6844

(52) U.S. Cl. CPC C07K 14/245; C12N 9/1276; C12N 9/78; C12N 15/902; C12Q 1/6823; C12Q 1/6844; C07K 2319/80; C12Y 207/07049; C12Y 305/04004; C12Y 305/04005

(57) ABSTRACT

Systems, methods and composition for targeting polynucleotides are detailed herein. In particular, engineered DNA-targeting systems comprising novel TnpB polypeptides and a reprogrammable targeting nucleic acid component and methods and application of use are provided.

### CROSS-REFERENCE TO RELATED APPLICATIONS

This application is a continuation application of PCT/US2023/076591, filed Oct. 11, 2023, which claims the benefit of U.S. Provisional Application No. 63/415,172, filed Oct. 11, 2022. The entire contents of the above-identified applications are hereby fully incorporated herein by reference.

### STATEMENT AS TO FEDERALLY SPONSORED RESEARCH

This invention was made with government support under Grant Nos. HL141201 and HG009761 awarded by The National Institutes of Health. The government has certain rights in the invention.

### SEQUENCE LISTING

This application contains a sequence listing filed in electronic form as an xml file entitled BROD-5720US_ST26.xml, created on Apr. 2, 2025, and having a size of 335,583 bytes. The content of the sequence listing is incorporated herein in its entirety.

### TECHNICAL FIELD

The subject matter disclosed herein is generally directed to systems, methods and compositions used for targeted gene modification and nucleic acid editing utilizing systems comprising TnpB polypeptides.

### BACKGROUND

While there are genome-editing techniques available for producing targeted genome perturbations, there remains a need for new genome engineering technologies that employ novel strategies and molecular mechanisms and are affordable, easy to set up, scalable, and amenable to targeting multiple positions within the genome. Additional desirable tools in genome engineering and biotechnology would further advance the art.

Citation or identification of any document in this application is not an admission that such a document is available as prior art to the present invention.

### SUMMARY

In one aspect, the present disclosure provides a non-naturally occurring, engineered composition comprising a) a TnpB polypeptide, a MazE polypeptide or domain and a nucleic acid component comprising a scaffold and a reprogrammable spacer sequence, the nucleic acid component molecule capable of forming a complex with the TnpB polypeptide and directing sequence-specific binding of a target polynucleotide, and wherein the MazE polypeptide or domain is linked to or otherwise capable of complexing with the TnpB polypeptide and/or nucleic acid component.

In example embodiments, the TnpB polypeptide comprises about 200 to about 500 amino acids. In example embodiments, the reprogrammable spacer sequence comprises a spacer of 10 nucleotides to 30 nucleotides in length. In example embodiments, the nucleic acid component molecule comprises a scaffold of about 80 to 200 nucleotides in length. In example embodiments, the TnpB complex binds a target adjacent motif (TAM) sequence 5′ of the target polynucleotide. In example embodiments, the TAM sequence comprises TCA. In example embodiments, the TAM sequence comprises TTCAN. In example embodiments, the target polynucleotide is DNA.

The composition of any one of the preceding claims, further comprising a homologous recombination donor template comprising a donor sequence for insertion into a target polynucleotide. In example embodiments, the composition further comprises a functional domain associated with the TnpB protein. In example embodiments, the functional domain is a transposase, an integrase, a nucleobase deaminase, a reverse transcriptase, a recombinase, an integrase, a topoisomerase, a retrotransposon, phosphatase, polymerase, ligase, helitron, a helicase, a methylase, a demethylase, a translation activater, a translation repressor, a transcription activator, a transcription repressor, a transcription release factor, a chromatin modifier, a histone modifier, a nuclease. In example embodiments, the TnpB polypeptide and MazE polypeptide are selected from Table 1.

In preferred embodiments, the engineered nucleic acid component comprises of an engineered OMEGA guide molecule. In example embodiments, the engineered OMEGA guide molecule is modified to: (i) further comprise one or more of a riboswitch sequence, a toehold switch sequence, or a ribozyme sequence; (ii) further comprise one or more nucleotide modifications; (iii) have a 5′ truncation, a 3′ truncation, or both, and wherein the truncations; or (iii) a combination thereof. In example embodiments, the riboswitch sequence is inserted on a 5′ end, a 3′ end, or an internal position of the OMEGA guide molecule. In example embodiments, the riboswitch sequence is configured to bind a small molecule and switch the guide molecule to an active structural conformation upon binding of the small molecule. In example embodiments, the small molecule is a synthetic small molecule or an endogenous small molecule produced by a cell. In example embodiments, the endogenous small molecule is indicative of a disease state.

In example embodiments, the toehold switch sequence is inserted on a 5′ end, a 3′ end, or an internal position of the OMEGA guide molecule. In example embodiments, the internal position is an exposed loop of the guide molecule. In example embodiments, the toehold switch is configured to bind an oligonucleotide and (i) switch the guide molecule from an in-active structural conformation to an active structural conformation upon binding the oligonucleotide, or (ii) switch the guide molecule from an active structural conformation to an in-active structural conformation. In example embodiments, the oligonucleotide is a synthetic oligonucleotide or an endogenous oligonucleotide expressed by a prokaryotic or eukaryotic cell. In example embodiments, the oligonucleotide is indicative of a disease state.

In example embodiments, the ribozyme sequence is added to a 5′ or 3′ end of the guide molecule. In example embodiments, the ribozyme sequence is configured to cleave a 5′ cap from the guide molecule. In example embodiments, the 3′ truncation comprises a truncation of between 1 and 21 nucleotides. In example embodiments, the one or more modifications comprise a 5′ truncation of between 1 and 45 nucleotides.

In example embodiments, the engineered nucleic acid component comprises of a trans oligonucleotide-activated OMEGA guide system comprising an in-active truncated OMEGA guide molecule, wherein the truncated OMEGA guide molecule comprises a portion capable of hybridizing with a trans oligonucleotide to form an activated guide molecule capable of complexing with an OMEGA polypeptide and directing site-specific binding of the complex to a target oligonucleotide. In example embodiments, the truncated OMEGA guide molecule has a truncation on the 3′ end of the guide molecule. In example embodiments, the 3′ truncation includes a truncation of a portion of the guide molecule forming a 3′ stem loop. In example embodiments, the 3′ truncation comprises between 22 and 45 nucleotides.

In example embodiments, the in-activate truncated OMEGA guide molecule further comprises between 1 and 8 additional base pair insertions in the 3′ stem loop. In example embodiments, the trans oligonucleotide is an endogenous oligonucleotide expressed by a prokaryotic or eukaryotic cell. In example embodiments, the trans oligonucleotide is a mRNA, lncRNA, miRNA, or piRNA. In example embodiments, the trans oligonucleotide is specifically expressed in a disease state. In example embodiments, the trans oligonucleotide is a synthetic trans oligonucleotide configured to hybridize with the in-active truncated guide molecule. In example embodiments, the trans oligonucleotide comprises a NN(U/C)U(A/G) motif.

In one aspect, the present disclosure provides a vector system comprising one or more vectors encoding the TnpB polypeptide and the nucleic acid component of any of the preceding claims. In one aspect, the present disclosure provides an engineered cell comprising any composition as described herein. In one aspect, the present disclosure provides a method of modifying a target polynucleotide sequence in a cell, comprising introducing into the cell the composition of any as described herein. In example embodiments, the modifying comprises cleaving a DNA polynucleotide. In example embodiments, the cleaving results in a 5′ overhang. In example embodiments, the cleavage occurs distal to a target-adjacent motif. In example embodiments, the cleavage occurs at the site of the spacer annealing site or 3′ of the target sequence. In example embodiments, the polypeptide and/or nucleic acid components are provided via one or more polynucleotides encoding the polypeptides and/or nucleic acid component(s), and wherein the one or more polynucleotides are operably configured to express the TnpB polypeptide and/or the nucleic acid component molecule.

In one aspect, the present disclosure provides a vector system comprising one or more vectors encoding the TnpB polypeptide and the ωRNA component as described herein. In one aspect, the present disclosure provides an engineered cell comprising the composition of any of those described herein. In one aspect, the present disclosure provides a method of modifying a target polynucleotide sequence in a cell, comprising introducing into the cell the composition of any of those described therein. In example embodiments, the modifying comprises cleaving a DNA polynucleotide. In example embodiments, the cleaving results in a 5′ overhang. In example embodiments, the cleavage occurs distal to a target-adjacent motif. In example embodiments, the cleavage occurs at the site of the spacer annealing site or 3′ of the target sequence. In example embodiments, the polypeptide and/or ωRNA components are provided via one or more polynucleotides encoding the polypeptides and/or ωRNA component(s), and wherein the one or more polynucleotides are operably configured to express the TnpB polypeptide and/or the ωRNA component molecule. In example embodiments, the one or more mutations include substitutions, deletions, and insertions.

In one aspect, the present disclosure provides an engineered, non-naturally occurring composition comprising: 1) a TnpB polypeptide, wherein the TnpB polypeptide is catalytically inactive, 2) a nucleotide deaminase associated with or otherwise capable of forming a complex with the TnpB protein, 3) a MazE polypeptide or domain; and 4) an nucleic acid component molecule capable of forming a complex with the TnpB protein and directing site-specific binding at a target sequence. In example embodiments, the TnpB and MazE is selected from Table 1. In example embodiments, the nucleotide deaminase is an adenosine deaminase or a cytidine deaminase.

In one aspect, the present disclosure provides for one or more polynucleotides encoding one or more components of the composition of any of those described herein. In one aspect, the present disclosure provides for one or more vectors encoding the one or more polynucleotides described herein. In one aspect, the present disclosure provides a cell or progeny thereof genetically engineered to express one or more components of any of those described herein.

In one aspect, the present disclosure provides a method of editing nucleic acids in target polynucleotides comprising delivering the composition of any of those described herein, the one or more polynucleotides of any of those described herein, or one or more vectors of those described herein to a cell or population of cells comprising the target polynucleotides. In example embodiments, the target polynucleotides are target sequences within genomic DNA. In example embodiments, the target polynucleotide is edited at one or more bases to introduce a G→A or C→T mutation.

In one aspect, the present disclosure provides an isolated cell or progeny thereof comprising one or more base edits made using the method of any one of as described herein. In one aspect, the present disclosure provides an engineered, non-naturally occurring composition comprising: 1) a catalytically dead TnpB polypeptide, 2) a reverse transcriptase associated with or otherwise capable of forming a complex with the TnpB polypeptide, 3) a MazE polypeptide or domain, and 4) an nucleic acid component molecule capable of forming a complex with the TnpB protein and directing site-specific binding of the complex to a target sequence of a target polynucleotide, the guide molecule further comprising a donor template encoding a donor sequence for insertion into the target polynucleotide.

In one aspect, the present disclosure provides a method of modifying target polynucleotides comprising; delivering the composition of any of those described herein, the one or more polynucleotides of any of those described herein, or the one or more vectors of any of those described herein to a cell, or population of cells, comprising the target polynucleotides, wherein the complex directs the reverse transcriptase to the target sequence and the reverse transcriptase facilitates insertion of a donor sequence encoded by the donor template from the ωRNA component molecule into the target polynucleotide. In example embodiments, insertion of the donor sequence comprises: 1) introduces one or more base edits; 2) corrects or introduces a premature stop codon; 3) disrupts a splice site; 4) inserts or restores a splice site; 5) inserts a gene or gene fragment at one or both alleles of the target polynucleotide; or 6) a combination thereof. In one aspect, the present disclosure provides an isolated cell or progeny thereof comprising the modifications made using the method of any of those described herein.

In one aspect, the present disclosure provides an engineered, non-naturally occurring composition comprising: 1) a TnpB polypeptide, 2) a non-LTR retrotransposon protein associated with or otherwise capable of forming a complex with the TnpB polypeptide, 3) a MazE polypeptide or domain, 4) a nucleic acid component molecule capable of forming a complex with the TnpB protein and directing site-specific binding of the complex to a target sequence of a target polynucleotide, the ωRNA molecule further comprising a donor template encoding a donor sequence for insertion into the target polynucleotide and located between two binding elements capable of forming a complex with the non-LTR retrotransposon protein.

In example embodiments, the TnpB protein is fused to the N-terminus of the non-LTR retrotransposon protein. In example embodiments, the TnpB protein is engineered to have nickase activity. In example embodiments, the nucleic acid component molecule directs the fusion protein to a target sequence 5′ of the targeted insertion site, and wherein the TnpB protein generates a strand break at the targeted insertion site. In example embodiments, the ωRNA component molecule directs the fusion protein to a target sequence 3′ of the targeted insertion site, and wherein the TnpB protein generates a strand break at the targeted insertion site. In example embodiments, the donor polynucleotide further comprises a polymerase processing element to facilitate 3′ end processing of the donor polynucleotide sequence. In example embodiments, the donor polynucleotide further comprises a homology region to the target sequence on the 5′ end of the donor construct, the 3′ end of the donor construct, or both. In example embodiments, the homology region is from 8 to 25 base pairs.

In one aspect, the present disclosure provides a method of modifying target polynucleotides comprising; delivering the composition of any one of those as describe herein, the one or more polynucleotides of those as describe herein, or one or more vectors of those as describe herein to a cell or population of cells comprising the target polynucleotides, wherein the complex directs the non-LTR retrotransposon protein to the target sequence and the non-LTR retrotransposon protein facilitates insertion of the donor polynucleotide sequence from the donor construct into the target polynucleotide. In example embodiments, insertion of the donor sequence: 1) introduces one or more base edits; 2) corrects or introduces a premature stop codon; 3) disrupts a splice site; 4) inserts or restores a splice site; 5) inserts a gene or gene fragment at one or both alleles of the target polynucleotide; or 6) a combination thereof.

In one aspect, the present disclosure provides an engineered, non-naturally occurring composition comprising: 1) A TnpB polypeptide, 2) an integrase protein associated with or otherwise capable of forming a complex with the TnpB polypeptide, and optionally a reverse transcriptase, and 3) an nucleic acid component molecule capable of forming a complex with the TnpB protein and directing site-specific binding of the complex to a target sequence of a target polynucleotide, the guide molecule further comprising a donor template encoding a donor sequence for insertion into the target polynucleotide and located between two binding elements capable of forming a complex with the integrase protein. In example embodiments, the TnpB protein is fused to the integrase protein and optionally the reverse transcriptase. In example embodiments, the TnpB protein is engineered to have nickase activity. In example embodiments, the nucleic acid component molecule directs the fusion protein to a target sequence, and wherein the TnpB protein generates a nick at the targeted insertion site. In example embodiments, the donor polynucleotide further comprises a homology region to the target sequence on the 5′ end of the donor construct, the 3′ end of the donor construct, or both.

In one aspect, the present disclosure provides a method of modifying target polynucleotides comprising; delivering the composition of any one of any of those as described, the one or more polynucleotides of any of those described herein, or one or more vectors of those described herein to a cell or population of cells comprising the target polynucleotides, wherein the complex directs the integrase protein to the target sequence and the integrase protein facilitates insertion of the donor polynucleotide sequence from the donor construct into the target polynucleotide. a method of modifying target polynucleotides comprising; delivering insertion of the donor sequence comprises: 1) introduces one or more base edits; 2) corrects or introduces a premature stop codon; 3) disrupts a splice site; 4) inserts or restores a splice site; 5) inserts a gene or gene fragment at one or both alleles of the target polynucleotide; or 6) a combination thereof.

In one aspect, the present disclosure provides a composition for detecting the presence of a target polynucleotide in a sample, comprising: one or more TnpB proteins possessing collateral activity; at least one nucleic acid component comprising a sequence capable of binding a target polynucleotide and designed to form a complex with the one or more TnpB proteins; a MazE polypeptide or domain, and a detection construct comprising a polynucleotide component, wherein the TnpB protein exhibits collateral nuclease activity and cleaves the polynucleotide component of the detection construct once activated by the target sequence; and optionally, isothermal amplification reagents.

In example embodiments, the TnpB and MazE is selected from Table 1. In example embodiments, the one or more TnpB proteins possess nuclease activity, in the temperature range of 45° C. to 60° C. In example embodiments, the isothermal amplification reagents are loop-mediated isothermal amplification (LAMP) reagents. In example embodiments, the LAMP reagents comprise LAMP primers. In example embodiments, the composition further comprises one or more additives to increase reaction specificity or kinetics. In example embodiments, the composition further comprises polynucleotide binding beads.

In one aspect, the present disclosure provides a method for detecting polynucleotides in a sample, the method comprising; contacting one or more target sequences with a TnpB, at least one ωRNA component capable of forming a complex with the TnpB and direct sequence-specific binding to one or more target polynucleotides and a detection construct, wherein the TnpB exhibits collateral nuclease activity and cleaves the detection construction once activated by the one or more target sequences; and detecting a signal from cleavage of the detection construction thereby detecting the one or more target polynucleotides. In example embodiments, the composition further comprises amplifying the target polynucleotides using isothermal amplification prior to the contacting step. In example embodiments, detection of amplified target polynucleotides by binding of the target polynucleotides to the TnpB complex occurs in the temperature range of 45° C. to 60° C. In example embodiments, the target polynucleotide is detected in one hour or less.

In one aspect, the present disclosure provides an expression construct comprising a polynucleotide encoding an TnpB polypeptide and a guide molecule, wherein guide molecule comprises a self-cleaving ribozyme sequence.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—shows example genomic loci architectures of TnpB systems in accordance with example embodiments disclosed herein.

FIG. 2—shows an example TnpB system in complex with a nucleic acid component and where an example MazE domain (referred to as HTH in this figure) contacts the TnpB complex.

FIG. 3—shows an example TnpB system in complex with a nucleic acid component and where an example MazE domain (referred to as HTH in this figure) contacts the TnpB complex.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4^{th} ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide engineered TnpB systems. The TnpB system comprises a TnpB polypeptide and a nucleic acid component capable of forming a complex with the TnpB polypeptide and directing the complex to a target polynucleotide. The TnpB systems and TnpB/nucleic acid component complexes may also be referred to herein as OMEGA (Obligate Mobile Element Guided Activity) systems or complexes, or 22 systems or complexes for short. TnpB systems are a distinct type of 22 system, which further include IscB, IsrB, and IshB systems. The nucleic acid component of (2 systems is structurally distinct from other RNA-guided nucleases, such as CRISPR-Cas systems, and may also be referred to as a ωRNA. In certain example embodiments, the TnpB systems are RNA-predominate, that is the nucleic acid component makes a larger contribution to the overall size of the TnpB complex relative to other RNA-guided nuclease systems such as CRISPR-Cas. Also, given the more minimal structural features of TnpB relative other known programmable nucleases such as CRISPR-Cas, the polynucleotide binding pocket is open and more accessible, which can facilitate greater access to and ability to manipulate, modify, edit, remove, or delete nucleotides at a target region on the bound polynucleotide. Disclosed herein are TnpB systems that may function as nuclease, nickases, or catalytically inactive polynucleotide binding proteins that can be coupled with other functional domains.

In one embodiment, the TnpB systems and related compositions may specifically target single-strand or double-strand DNA. In one embodiment, the TnpB system may bind and cleave double-strand DNA. In one embodiment, the TnpB system may bind to double-stranded DNA without introducing a break to either of the strands. In one embodiment, the TnpB polypeptides or nuclease/nucleic acid component complexes may open, disrupting the continuity of one of the two DNA strands, thereby introducing a nick of the double stranded DNA. In an embodiment, and without being bound by theory, the size and configuration of the TnpB systems allows exposure to the non-targeting strand, which may be in single-stranded form, to allow for the ability to modify, edit, delete or insert polynucleotides on the non-target strand. In an embodiment, this accessibility further allows for enhanced editing outcomes on the target and/or non-target strand, e.g., increased specificity, enhanced editing efficiency. The TnpB systems disclosed herein further comprise a MazE polypeptide or domain. In on example embodiment, the MazE domain comprises a HTH domain.

In another aspect, embodiments disclosed herein include applications of the compositions herein, including therapeutic and diagnostic compositions and uses. Delivery of the proteins and systems disclosed is also provided, including to a variety of cells and via a variety of delivery vehicles.

### TnpB Compositions

In one aspect, embodiments disclosed herein are directed to compositions comprising a TnpB and a ωRNA capable of forming a complex with the TnpB and directing site-specific binding of the TnpB to a target sequence on a target polynucleotide.

### TnpB Polypeptides

TnpB polypeptides of the present invention may comprise a Ruv-C-like domain. Exemplary TnpB sequences are shown in Table 1. The RuvC domain may be a split RuvC domain comprising RuvC-I, RuvC-II, and RuvC-III subdomains. The TnpB may further comprise one or more of a HTH domain, a bridge helix domain and a zinc finger domain. TnpB polypeptides do not comprise an HNH domain. In one example embodiment, TnpB proteins comprise, starting at the N-terminus a HTH domain, a RuvC-I sub-domain, a bridge helix domain, a RuvC-II sub-domain, a zinger finger domain, and a RuvC-III sub-domain. In one example embodiment, the RuvC-III sub-domain forms the C-terminus of the TnpB polypeptide.

In certain example embodiments, the TnpB polypeptides are between 175 and 800 amino acids in size, between 200 and 790 amino acids in size, between 200 and 780 amino acids in size, between 200 and 770 amino acids in size, between 200 and 760 amino acids in size, between 200 and 750 amino acids in size, between 200 and 740 amino acids in size, between 200 and 730 amino acids in size, between 200 and 720 amino acids in size, between 200 and 720 amino acids in size, between 200 and 710 amino acids in size, between 200 and 700 amino acids in size, between 200 and 690 amino acids in size, between 200 and 680 amino acids in size, between 200 and 670 amino acids in size, between 200 and 660 amino acids in size, between 200 and 650 amino acids in size, between 200 and 640 amino acids in size, between 200 and 630 amino acids in size, between 200 and 620 amino acids in size, between 200 and 610 amino acids in size, between 200 and 600 amino acids in size, between 200 and 590 amino acids in size, between 200 and 580 amino acids in size, between 200 and 570 amino acids in size, between 200 and 560 amino acid, between 200 between 550 amino acids, between 200 and 540 amino acids, between 200 and 530 amino acids, between 200 and 520 amino acids, between 200 and 510 amino acids, between 200 and 500 amino acids, between 200 and 490 amino acids, between 200 and 480 amino acids, between 200 and 470 amino acids, between 200 and 460 amino acids, between 200 and 450 amino acids, between 200 and 440 amino acids, between 200 and 430 amino acids, between 200 and 420 amino acids, between 200 and 410 amino acids, between 210 and 500 amino acids, between 220 and 500 amino acids. Between 230 and 500 amino acids, between 240 and 500 amino acids, between 250 and 500 amino acids, between 260 and 500 amino acids, between 270 and 500 amino acids, between 280 and 500 amino acids, between 290 and 500 amino acids, between 300 and 500 amino acids, between 250 and 490 amino acids, between 250 and 480 amino acids, between 250 and 490 amino acids, or between 250 and 600 amino acids. In one embodiment, the TnpB polypeptide is between 300 and 500 amino acids, or between 350 and 450 amino acids.

In one embodiment, the TnpB polypeptides may comprise a modified naturally occurring protein, functional fragment or truncated version thereof, or a non-naturally occurring protein. In one embodiment, the TnpB polypeptide comprises one or more domains originating from other TnpB polypeptides, more particularly originating from different organisms. In one embodiment, the TnpB polypeptides may be designed by in silico approaches. Examples of in silico protein design have been described in the art and are therefore known to a skilled person.

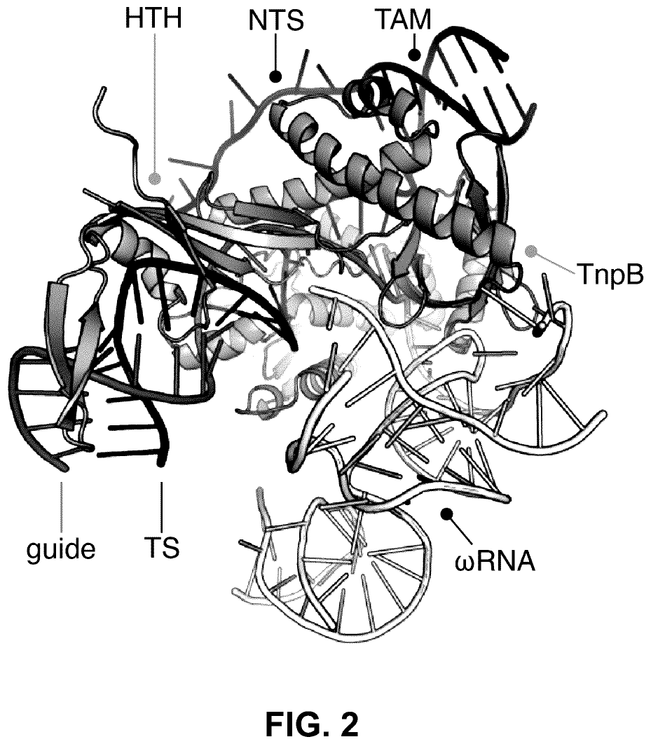

In one embodiment, the TnpB polypeptide is from Epsilonproteobacteria bacterium, or Actinoplanes lobatus strain DSM 43150, Actinomadura celluolosilytica strain DSM 45823, Actinomadura namibiensis strain DSM 44197, Alicyclobacillus macrosprangiidus strain DSM 17980, Lipingzhangella halophila strain DSM 102030, or Ktedonobacter recemifer. In one embodiment, the TnpB polypeptide is from Ktedonobacter racemifer, or comprises a conserved RNA region with similarity to the 5′ ITR of K. racemifer TnpB loci. See, e.g., Table 5, FIG. 2. In an aspect, the TnpB polypeptide encodes 5′ ITR/RNA (with RNA on the 3′ strand), TnpB (3′ strand), and lastly 3′ ITR. In one example embodiment, the TnpB may comprise a Fanzor protein, TnpB homologs, found in eukaryotic genomes.

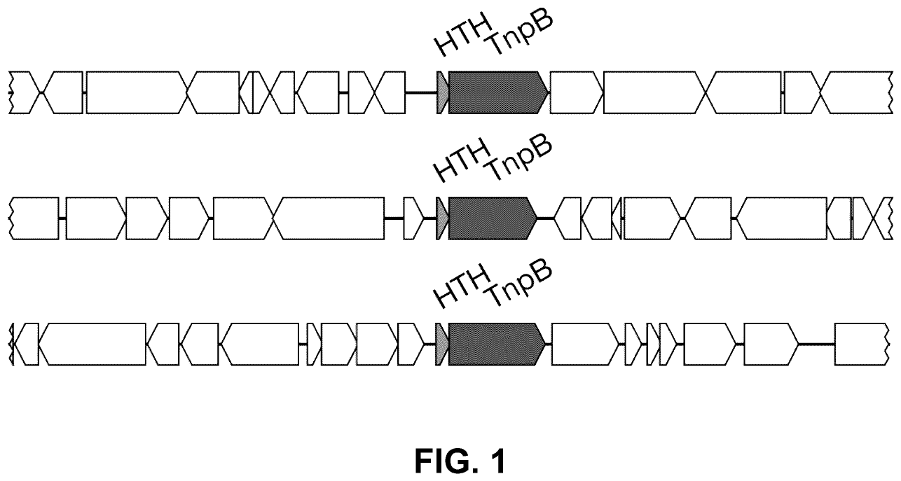

The TnpB polypeptides also encompasses homologs or orthologs of TnpB polypeptides whose sequences are specifically described herein. The terms “ortholog” and “homolog” are well known in the art. By means of further guidance, a “homolog” of a protein as used herein is a protein of the same species which performs the same or a similar function as the protein it is a homolog of. Homologous proteins may but need not be structurally related, or are only partially structurally related. An “ortholog” of a protein as used herein is a protein of a different species which performs the same or a similar function as the protein it is an orthologue of. Orthologous proteins may be, but may not always be, structurally related or are only partially structurally related. In particular embodiments, the homolog or ortholog of a TnpB polypeptide such as referred to herein has a sequence homology or identity of at least 80%, at least 81%, at least 82%, at least 83%, at least 84% at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% with a TnpB polypeptide, more specifically with a TnpB sequence identified in Table 1A, 1B, 1C, 5, or FIG. 1. In particular embodiments, a homolog or ortholog is identified according to its domain structure and/or function. In embodiments, the homolog or ortholog comprises catalytic residues and/or domains as defined herein, including as identified in FIGS. 1 and 18. Sequence alignments conducted as described herein, as well as folding studies and domain predictions as taught herein can aid in the identification of a homolog or ortholog with the structural and functional characteristics identifying TnpB polypeptides, particularly those with conserved residues, including catalytic residues, and domains of TnpB polypeptides.

In one embodiment, the TnpB loci comprises inverted terminal repeats (ITRs). An inverted terminal repeat may be present on the 5′ or 3′ end of the TnpB sequence. In an aspect, the inverted terminal repeat may comprise between about 20 to about 40 nucleotides, for example, about 20, 21, 22, 23, 24, 25, 26, 27, 28, 29, 30, 31, 32, 33, 34, 35, 36, 37, 38, 39, or 40 nucleotides. In embodiments, the ITR comprises about 25 to 35 nucleotides, about 28 to 32 nucleotides. In an aspect, the ITR shares similarity with one or more inverted terminal repeats with sequences encoding IscB polypeptides. In one embodiment, the 5′ ITR or 3′ITR of TnpB has a sequence homology or identity of at least 80%, at least 85%, at least 90%, at least 95%, at least 96%, at least 97% at least 98% or at least 99% identity with an IscB 5′ ITR or 3′ ITR. In an embodiment, the 5′ ITR of the TnpB is homologous to the 5′ ITR of the IscB. Exemplary IscB ITRs are disclosed in Altae-Tran et al., Science 9 Sep. 2021, 374:6563, pp. 57-65; doi: 10.1126/science.abj685, specifically incorporated herein by reference in its entirety, including supplementary materials Data S1 to S4 and Tables S1 to S6.

In one embodiment, the TnpB loci comprises a region of high conservation beyond the sequence encoding the polypeptide that indicates the presence of RNA at the 5′ end of the TnpB loci. In an aspect, the region upstream of the 5′ ITR of TnpB comprises a region encoding an RNA species that comprises a guide sequence.

A chimeric enzyme can comprise a first fragment and a second fragment, and the fragments can be of TnpB polypeptide orthologs of organisms of a genus or of a species, e.g., the fragments are from TnpB polypeptide orthologs of different species.

### RuvC Domain

In one embodiment, the TnpB polypeptide comprises at least at least one RuvC-like nuclease domain. The RuvC domain may comprise conserved catalytic amino acids indicative of the RuvC catalytic residue. In an example embodiment, the RuvC catalytic residue may be referenced relative to 186D, 270E or 354D of TnpB polypeptide 488601079 of Table 1A; to 172D, 254E, or 337D of TnpB polypeptide 297565028 of Table 1A; or to 179D, 268E, or 351D of TnpB polypeptide 257060308 of Table 1A. The catalytic residue may be referenced relative to 195D, 277E, or 361D of the sequence alignment in FIG. 1. In an aspect, the RuvC domain may comprise multiple subdomains, e.g., RuvC-I, RuvC-II and RuvC-III. The subdomains may be separated by intervening amino acid sequence of the protein. An exemplary domain architecture of an example TnpB polypeptide is shown in FIG. 18A.

In one embodiment, examples of the RuvC domain include any polypeptides a structural similarity and/or sequence similarity to a RuvC domain described in the art. In some examples, the RuvC domain may have an amino acid sequence that share at least 50%, at least 55%, at least 60%, at least 65%, at least 70%, at least 75%, at least 80%, at least 85%, at least 90%, at least 95%, at least 99%, or 100% sequence identity with RuvC domains known in the art.

In some examples, the RuvC domain comprise RuvC-I sub-domain, RuvC-II sub-domain, and RuvC-III sub-domain. Examples of the RuvC-I sub-domain also include any polypeptides having structural similarity and/or sequence similarity to a RuvC-I domain described in the art. For example, the RuvC-I domain may share a structural similarity and/or sequence similarity to a RuvC-I found in bacterial or archaeal species In some examples, the RuvC domain may have an amino acid sequence that share at least 50%, at least 55%, at least 60%, at least 65%, at least 70%, at least 75%, at least 80%, at least 85%, at least 90%, at least 95%, at least 99%, or 100% sequence identity with RuvC-I domain. The RuvC-II domain also include any polypeptides a structural similarity and/or sequence similarity to a RuvC-II domain described in the art. In some examples, the RuvC domain may have an amino acid sequence that share at least 50%, at least 55%, at least 60%, at least 65%, at least 70%, at least 75%, at least 80%, at least 85%, at least 90%, at least 95%, at least 99%, or 100% sequence identity with RuvC-II domains. The RuvC-III domain also include any polypeptides a structural similarity and/or sequence similarity to a RuvC-III domain described in the art. In some examples, the RuvC domain may have an amino acid sequence that share at least 50%, at least 55%, at least 60%, at least 65%, at least 70%, at least 75%, at least 80%, at least 85%, at least 90%, at least 95%, at least 99%, or 100% sequence identity with RuvC-III domains.

For example, and as described in the art (e.g., Crystal structure of Cas9 in complex with nucleic acid component molecule and target DNA, Nishimasu et al. Cell, 2014) a RuvC may consist of a six-stranded mixed β-sheet (β1, β2, β5, β11, β14 and β17) flanked by α-helices (α33, α34 and α39-α45) and two additional two-stranded antiparallel β-sheets (β3/4 and β15/β16). It has been described that some RuvC domains shares structural similarity with the retroviral integrase superfamily members characterized by an RNase H fold, such as Escherichia coli RuvC (PDB code 1HJR, 14% identity, root-mean-square deviation (rmsd) of 3.6 Å for 126 equivalent Ca atoms) and Thermus thermophilus RuvC (PDB code 4LD0, 12% identity, rmsd of 3.4 Å for 131 equivalent Ca atoms). E. coli RuvC is E. coli RuvC is a 3-layer alpha-beta sandwich containing a 5-stranded beta-sheet sandwiched between 5 alpha-helices. RuvC nucleases have four catalytic residues (e.g., Asp7, Glu70, His143 and Asp146 in T. thermophilus RuvC), and cleave Holliday junctions (or structurally analogous cruciform junctions) through a two-metal mechanism. Asp10 (Ala), Glu762, His983 and Asp986 of the Cas9 RuvC domain are located at positions similar to those of the catalytic residues of T. thermophilus RuvC. The RuvC-like domain of the TnpB polypeptides may comprise 1, 2, 3 or 4 of the catalytic residues.

In embodiments, the TnpB polypeptide is a nuclease. In one embodiment, the TnpB and nucleic acid component can direct sequence-specific nuclease activity. The cleavage may result in a 5′ overhang. The cleavage may occur distal to a target-adjacent motif (TAM), and may occur at the site of the spacer (guide) annealing site or 3′ of the target sequence. In an aspect, the TnpB cleaves at multiple positions within and beyond the nucleic acid component annealing site. In an aspect, DNA cleavage occurs 15, 16, 17, 18, 19, 20, 21, 22, 23, 24, 25 or more base pairs distal to the TAM and results in a 5′ overhang.

In an embodiment, the TnpB polypeptide is active, i.e., possesses nuclease activity, over a temperature range of from about 37° C. to about 80° C. In an embodiment, the TnpB polypeptide is active from about 37° C. to about 75° C., from about 37° C. to about 70° C., from about 37° C. to about 65° C., from about 37° C. to about 60° C., from about 37° C. to about 55° C., from about 37° C. to about 50° C., from about 37° C. to about 45° C. In an example embodiment, the TnpB polypeptide is active in the range of 37° C. to 65° C. In an example embodiment, the TnpB polypeptide is active in the range of 45° C. to 65° C. In an example embodiment, the TnpB polypeptide is active in the range of 45° C. to 60° C. In a further example embodiment, the TnpB polypeptide is the TnpB protein selected from Actinomadura cellulosilytica strain DSM 45823, Actinomadura namibiensis strain DSM 44197, Actinoplanus lobatus strain DSM 43150 (TnpB-1 and TnpB-2), Lipingzhangella halophila strain DSM 102030, Ktedonobacter racemifer, and Epsilonproteobacteria bacterium QNF01000004_Extraction (reversed). In another example embodiment, the TnpB polypeptide is from Alicyclobacillus macrosporangiidus strain DSM 17980. In an example embodiment, the Alicyclobacillus macrosporangiidus strain DSM 17980 TnpB protein is most active in the range of 45° C. to 60° C. (FIG. 22).

In one embodiment, the TnpB polypeptide displays collateral activity, also referred to as trans cleavage, where upon activation and cleavage of its cognate target, non-specific cleave of non-cognate nucleic acid occurs. In an aspect, the TnpB polypeptide possesses collateral activity once triggered by target recognition. In an aspect, upon binding to the target sequence, the TnpB polypeptide will non-specifically cleave polynucleotide sequences, e.g., DNA. The target-activated nonspecific nuclease activity of TnpB is also referred to herein as collateral activity.

In an embodiment, the TnpB protein displays nuclease activity towards both ssDNA and dsDNA target sequences. In an embodiment, the TnpB protein displays nuclease activity towards both ssDNA and dsDNA wherein a TAM may not be necessary to cut a ssDNA target (FIG. 23).

In embodiments, the TnpB polypeptide is a nuclease. In one embodiment, the TnpB and nucleic acid component molecule can direct sequence-specific nuclease activity. The TnpB polypeptides provided herein may also exhibit RNA-guided recombinase activity. The homology to the RuvC domain and relatedness to the DDE family of recombinases indicate potential recombinase activity. In an embodiment some TnpB polypeptides detailed herein may naturally exhibit, or be engineered to exhibit, a lack of nuclease activity, or reduced nuclease activity, and are provided with a functional domain as detailed herein, for example, nucleotide deaminases, reverse transcriptases, transposable elements, e.g., transposase, integrase, recombinase, allowing for RNA-guided target specific modifications.

### Example TnpB Polypeptides

In certain example embodiments, the TnpB protein may comprise a sequence as set forth in Table 1A, Table 1B, or Table 1C. In Table 1A, provided are the native TnpB amino acid sequences for A. cellulosilytica, A. lobatus TnpB-1, H. alba, A. namibiensis, A. umbrina and Epsilonproteobacteria bacterium 10_QNFX01000004 extraction reversed which all start (+1 position) with a valine (GTG) but as is well known in the art is translated as a methionine because of the peculiar nature of the initiator tRNA.

TABLE 1
Exemplary TnpB and MazE Sequences

| | TnpB_seq | MazE_seq |
|---|---|---|
| contig_id | SEQ ID NO | SEQ ID NO |
| 0180301_10006133 | 1 | |
| DTCV01000040.1 | 2 | |
| Ga0081425_146485 | 3 | |
| Ga0065719_107510 | 4 | |
| 0180302_10020142 | 5 | |
| a0326760_1000146 | 6 | |
| a0326762_1001126 | 7 | |
| 0180301_10000028 | 8 | 9 |
| CP002426.1 | 10 | 11 |
| CP001402.1 | 12 | 13 |
| CP001404.1 | 14 | |
| CP001404.1 | 15 | 16 |
| BA000023.2 | 17 | 18 |
| CP001404.1 | 19 | 20 |
| NBVN01000002.1 | 21 | 22 |
| WYEO01000063.1 | 23 | |
| Ga0326758_100006 | 24 | 25 |
| BBDO01000010.1 | 26 | 27 |
| Ga0326758_100010 | 28 | 29 |
| 0180301_10003051 | 30 | 31 |
| 0180301_10003263 | 32 | 33 |
| NJEC01000211.1 | 34 | 35 |
| QMWZ01000063.1 | 36 | 37 |
| OGCG01006409.1 | 38 | 39 |
| scf1118683610783 | 40 | 41 |
| QMWV01000213.1 | 42 | 43 |
| DQYJ01000280.1 | 44 | 45 |
| 0180301_10041172 | 46 | 47 |
| Ga0326765_101204 | 48 | 49 |
| Ga0326758_102284 | 50 | 51 |
| Ga0326758_102980 | 52 | 53 |
| 0180300_10000035 | 54 | 55 |
| a0063354_1000266 | 56 | 57 |
| CP002051.1 | 58 | 59 |
| 0164310_10002417 | 60 | 61 |
| 0180300_10001883 | 62 | 63 |
| 0180301_10005587 | 64 | 65 |
| 0180300_10003564 | 66 | 67 |
| 0164318_10018220 | 68 | 69 |
| 0180301_10011776 | 70 | 71 |
| NJEC01000162.1 | 72 | 73 |
| 0164311_10014497 | 74 | 75 |
| CP001857.1 | 76 | |
| QMWV01000014.1 | 77 | |
| 0180301_10000160 | 78 | |
| NJDT01000039.1 | 79 | |
| QMSP01000016.1 | 80 | |
| JAADDN010000174.1 | 81 | |
| 0180300_10001082 | 82 | 83 |
| JACNFM010000308.1 | 84 | 85 |
| DQWM01000055.1 | 86 | 87 |
| LCWN02000003.1 | 88 | 89 |
| CP002100.1 | 90 | 91 |
| a0326760_1000014 | 92 | 93 |
| a0326760_1000142 | 94 | 95 |
| 0315272_10000973 | 96 | 97 |
| 0180301_10002355 | 98 | 99 |
| a0197143_1001366 | 100 | 101 |
| DTCV01000040.1 | 102 | 103 |
| OFEJ01001365.1 | 104 | 105 |
| QMRZ01000080.1 | 106 | 107 |
| DQXH01000129.1 | 108 | 109 |
| Ga0065719_104821 | 110 | 111 |
| 0180301_10017624 | 112 | 113 |
| NJDT01000144.1 | 114 | 115 |
| a0326760_1004649 | 116 | 117 |
| Ga0065719_116852 | 118 | 119 |
| 0180302_10015610 | 120 | 121 |
| 0180301_10031888 | 122 | 123 |
| 0180302_10018094 | 124 | 125 |
| a0326760_1008489 | 126 | 127 |
| CP000493.1 | 128 | |
| 0180301_10002641 | 129 | |
| DRZR01000193.1 | 130 | |
| CP001403.1 | 131 | |
| AHJK01000001.1 | 132 | |
| Ga0326758_100007 | 133 | |
| AHJK01000001.1 | 134 | |
| CP001403.1 | 135 | |
| JH597761.1 | 136 | |
| CP001400.1 | 137 | |
| CP029289.1 | 138 | |
| BBCT01000001.1 | 139 | |
| scf1118683610393 | 140 | |
| 0180301_10003807 | 141 | |
| OSPB_contig00595 | 142 | |
| 0180301_10028196 | 143 | |
| 0180302_10015518 | 144 | |
| DRAZ01000230.1 | 145 | |
| OGCG01008231.1 | 146 | |
| CP001857.1 | 147 | 148 |
| QMSP01000021.1 | 149 | 150 |
| OGCE01001859.1 | 151 | 152 |
| OGCB01001092.1 | 153 | 154 |
| Ga0065719_100868 | 155 | 156 |
| 0180301_10000741 | 157 | 158 |
| 0180301_10003768 | 159 | 160 |
| QMWV01000218.1 | 161 | 162 |
| QMSP01000023.1 | 163 | 164 |
| 0180302_10000030 | 165 | 166 |
| QMRQ01000058.1 | 167 | 168 |
| 0180302_10003229 | 169 | 170 |
| 0164318_10018220 | 171 | |
| 0164311_10006234 | 172 | |
| 0164318_10032829 | 173 | 174 |
| 0180301_10022711 | 175 | 176 |
| QMSA01000086.1 | 177 | |
| DQYF01000334.1 | 178 | 179 |
| CP008887.1 | 180 | 181 |
| QMUI01000016.1 | 182 | 183 |
| LLYW01000047.1 | 184 | 185 |
| 0180302_10022843 | 186 | |
| a0081762_1010554 | 187 | |
| 0180301_10034909 | 188 | |
| 0164315_10048720 | 189 | |
| 0180301_10000349 | 190 | |
| Ga0065719_100722 | 191 | |

The TnpB polypeptide may comprise one or more modifications. As used herein, the term “modified” with regard to a TnpB polypeptide generally refers to a TnpB polypeptide having one or more modifications or mutations (including point mutations, truncations, insertions, deletions, chimeras, fusion proteins, etc.) compared to the wild type counterpart from which it is derived. By derived is meant that the derived enzyme is largely based, in the sense of having a high degree of sequence or structural homology with, a wildtype enzyme, but that it has been mutated (modified) in some way as known in the art or as described herein.

The modified proteins, e.g., modified TnpB polypeptide may be catalytically inactive (also referred as dead). As used herein, a catalytically inactive or dead nuclease may have reduced, or no nuclease activity compared to a wildtype counterpart nuclease. In some cases, a catalytically inactive or dead nuclease may have nickase activity. In some cases, a catalytically inactive or dead nuclease may not have nickase activity. Such a catalytically inactive or dead nuclease may not make either double-strand or single-strand break on a target polynucleotide but may still bind or otherwise form complex with the target polynucleotide.

In an embodiment, eukaryotic homologues of bacterial TnpB may be utilized in the present invention. These TnpB-like proteins, Fanzor 1 and Fanzor 2, while having a shared amino acid motif in their C-terminal half regions, are variable in their N terminal regions. See, Bao et al., Homologues of bacterial TnpB_IS605 are widespread in diverse eukaryotic transposable elements. Mobile DNA 4, 12 (2013). Doi: 10.1186/1759-8753-4-12. In an aspect, the conserved sequence between TnpB and fanzor comprise D-X (125, 275)-[TS]-[TS]-X-X-[C4 zinc finger]-X (5,50)-RD. Fanzor proteins, in addition to varying in their N-terminal region from TnpB have higher diversity, with Fanzor proteins associated with different transposons and compositions. With Applicant's discovery of the nucleic acid component and mechanism for reprogramming TnpB polypeptide activity, the similarity of the Fanzor systems may allow for similar use and applications.

In one embodiment, the modifications of the TnpB polypeptide may or may not cause an altered functionality. By means of example, modifications which do not result in an altered functionality include for instance codon optimization for expression into a particular host, or providing the nuclease with a particular marker (e.g., for visualization). Modifications with may result in altered functionality may also include mutations, including point mutations, insertions, deletions, truncations (including split nucleases), etc., as well as chimeric nucleases (e.g., comprising domains from different orthologues or homologues) or fusion proteins. Fusion proteins may without limitation include, for instance, fusions with heterologous domains or functional domains (e.g., localization signals, catalytic domains, etc.). In one embodiment, various different modifications may be combined (e.g., a mutated nuclease which is catalytically inactive and which further is fused to a functional domain, such as for instance to induce DNA methylation or another nucleic acid modification, such as including without limitation, a break (e.g., by a different nuclease (domain)), a mutation, a deletion, an insertion, a replacement, a ligation, a digestion, a break or a recombination). As used herein, “altered functionality” includes without limitation an altered specificity (e.g., altered target recognition, increased (e.g., “enhanced” TnpB polypeptide) or decreased specificity, or altered TAM recognition), altered activity (e.g., increased or decreased catalytic activity, including catalytically inactive nucleases or nickases), and/or altered stability (e.g., fusions with destabilization domains). Examples of all these modifications are known in the art. It will be understood that a “modified” nuclease as referred to herein, and in particular a “modified” TnpB polypeptide or system or complex preferably still has the capacity to interact with or bind to the polynucleic acid (e.g., in complex with the nucleic acid component molecule). Such modified TnpB polypeptide can be combined with the deaminase protein or active domain thereof as described herein.

In one embodiment, an unmodified TnpB polypeptides may have cleavage activity. In one embodiment, the TnpB polypeptides may direct cleavage of one or both nucleic acid (DNA or RNA) strands at the location of or near a target sequence, such as within the target sequence and/or within the complement of the target sequence or at sequences associated with the target sequence. In one embodiment, the TnpB polypeptides may direct cleavage of one or both DNA or RNA strands within about 1, 2, 3, 4, 5, 6, 7, 8, 9, 10, 15, 20, 25, 50, 100, 200, 500, or more base pairs or nucleotides from the first or last nucleotide of a target sequence. In one embodiment, the cleavage may be staggered, i.e., generating sticky ends. In one embodiment, the cleavage is a staggered cut with a 5′ overhang. In one embodiment, the cleavage is a staggered cut with a 5′ overhang of 1 to 5 nucleotides, preferably of 4 or 5 nucleotides. In particular embodiments, the TnpB polypeptides cleave DNA strands.

In one embodiment, a TnpB polypeptide may be mutated with respect to a corresponding wild-type enzyme such that the mutated TnpB lacks the ability to cleave one or both strands of a target polynucleotide containing a target sequence. As a further example, two or more catalytic domains of a TnpB polypeptide (e.g., RuvC) may be mutated to produce a mutated TnpB polypeptide substantially lacking all DNA cleavage activity. In one embodiment, a TnpB polypeptide may be considered to substantially lack all polynucleotide cleavage activity when the polynucleotide cleavage activity of the mutated enzyme is no more than 25%, no more than 10%, no more than 5%, no more than 1%, no more than 0.1%, no more than 0.01% of the nucleic acid cleavage activity of the non-mutated form of the enzyme; an example can be when the nucleic acid cleavage activity of the mutated form is nil or negligible as compared with the non-mutated form.

In one embodiment, the TnpB polypeptide may comprise one or more modifications resulting in enhanced activity and/or specificity, such as including mutating residues that stabilize the targeted or non-targeted strand. In one embodiment, the altered or modified activity of the engineered TnpB polypeptide comprises increased targeting efficiency or decreased off-target binding. In one embodiment, the altered activity of the engineered TnpB polypeptide comprises modified cleavage activity. In one embodiment, the altered activity comprises increased cleavage activity as to the target polynucleotide loci. In one embodiment, the altered activity comprises decreased cleavage activity as to the target polynucleotide loci. In one embodiment, the altered activity comprises decreased cleavage activity as to off-target polynucleotide loci. In one embodiment, the modified nuclease comprises a modification that alters association of the protein with the nucleic acid molecule comprising RNA, or a strand of the target polynucleotide loci, or a strand of off-target polynucleotide loci. In an aspect of the invention, the engineered TnpB polypeptide comprises a modification that alters formation of the TnpB polypeptide and related complex. In one embodiment, the altered activity comprises increased cleavage activity as to off-target polynucleotide loci. Accordingly, in one embodiment, there is increased specificity for target polynucleotide loci as compared to off-target polynucleotide loci. In other embodiments, there is reduced specificity for target polynucleotide loci as compared to off-target polynucleotide loci. In one embodiment, the mutations result in decreased off-target effects (e.g., cleavage or binding properties, activity, or kinetics), such as in case for TnpB polypeptide for instance resulting in a lower tolerance for mismatches between target and the ωRNA. Other mutations may lead to increased off-target effects (e.g., cleavage or binding properties, activity, or kinetics). Other mutations may lead to increased or decreased on-target effects (e.g., cleavage or binding properties, activity, or kinetics). In one embodiment, the mutations result in altered (e.g., increased or decreased) activity, association or formation of the functional nuclease complex. Examples mutations include mutation of negative or neutral residues to positively charged residues, or positively charged residues to neutral or neutral residues to negative residues and/or (evolutionary) conserved residues, such as conserved positively charged residues, in order to enhance specificity. See, e.g., Zhou et al., Chem Rev. 2018 Feb. 28: 118(4): 1691-1741, doi: 10.1021/acs/chemrev.7b00305 (discussing electrostatic interactions in protein binding and effects of amino acid mutations on such electrostatic interactions). In one embodiment, such residues may be mutated to uncharged residues, such as alanine. Because the TnpB polypeptide interacts with guide or bound DNA over the length of the TnpB polypeptide, mutation of residues across the TnpB polypeptide may be utilized for altered activity. In an aspect, the TnpB polypeptide residues for mutation are altered based on amino acid sequence positions of Deinococcus radiodurans ISDra2, see, e.g., Karvelis et al., Nature 599, 692-696(2021). The ISDra2 amino acid may comprise the sequence:

(SEQ ID NO: 192)
MIRNKAFVVRLYPNAAQTELINRTLGSARFVYNHFLARRIAAYKESGK
GLTYGQTSSELTLLKQAEETSWLSEVDKFALQNSLKNLETAYKNFFRT
VKQSGKKVGFPRFRKKRTGESYRTQFTNNNIQIGEGRLKLPKLGWVKT
KGQQDIQGKILNVTVRRIHEGHYEASVLCEVEIPYLPAAPKFAAGVDV
GIKDFAIVTDGVRFKHEQNPKYYRSTLKRLRKAQQTLSRRKKGSARYG
KAKTKLARIHKRIVNKRQDFLHKLTTSLVREYEIIGTEHLKPDNMRKN
RRLALSISDAGWGEFIRQLEYKAAWYGRLVSKVSPYFPSSQLCHDCGF
KNPEVKNLAVRTWTCPNCGETHDRDENAALNIRREALVAAGISDTLNA
HGGYVRPASAGNGLRSENHATLVV.

In an embodiment, one or more residues are mutated to alter the TAM specificity of the TnpB polypeptide. In an aspect, the one or more mutations correspond to one or more of 52TYR, 53GLY, 56SER, 57SER, 60THR, 72SER, 75ASP, 76LYS, 77PHE, 80GLN, 84LYS, 119ARG, 121GLN, 122PHE, 123THR, 124ASN, 125ASN, 126ASN, 137PRO, 138LYS, 153LYS, 155LEU, and 172LEU based on amino acid sequence positions of ISDra2.

In one embodiment, one or more residues are mutated to alter the specificity and/or activity of the TnpB selected from one or more of 6ALA, 7PHE, 8VAL, 9VAL, 10ARG, 11LEU, 12TYR, 35PHE, 36LEU, 39ARG, 40ILE, 42ALA, 43TYR, 46SER, 47GLY, 48LYS, 49GLY, 50LEU, 51THR, 52TYR, 95ARG, 96THR, 97VAL, 98LYS, 99GLN, 100SER, 101GLY, 102 LYS, 103LYS, 104VAL, 105GLY, 106PHE, 107PRO, 108ARG, 109PHE, 110ARG, 111LYS, 112LYS, 113ARG, 114THR, 115GLY, 116GLU, 117SER, 118TYR, 119ARG, 120THR, 121GLN, 154ILE, 155LEU, 156ASN, 157VAL, 158THR, 159VAL, 160ARG, 161ARG, 162 ILE, 163HIS, 164GLU, 165GLY, 166HIS, 167TYR, 168GLU, 169ALA, 170SER, 171VAL, 172LEU, 173CYS, 174GLU, 215TYR, 216ARG, 217SER, 218THR, 219LEU, 220LYS, 221ARG, 222LEU, 223ARG, 224LYS, 225ALA, 226GLN, 227GLN, 228THR, 229LEU, 230SER, 231ARG, 232ARG, 233LYS, 234LYS, 235GLY, 236SER, 237ALA, 238ARG, 239YR, 240GLY, 241LYS, 242ALA, 243LYS, 244THR, 245LYS, 246LEU, 247ALA, 248ARG, 249ILE, 250HIS, 251LYS, 252ARG, 253ILE, 254VAL, 283ASP, 284ASN, 285MET, 286ARG, 287LYS, 288ASN, 289ARG, 290ARG, 291LEU, 292ALA, 293LEU, 294SER, 295ILE, 296SER, and 297ASP based on amino acid sequence positions of ISDra2.

Without being bound by a particular scientific theory, it is believed that Type V CRISPR-Cas systems evolved from TnpB systems. Type V systems are known to possess collateral activity in vitro against single-stranded DNA, see, e.g., Chen et al., Science. 2018 Apr. 27; 360(6387): 436-439.

### Specialty TnpB Systems

In one embodiment, the system is a TnpB-based system that is capable of performing a specialized function or activity. For example, the TnpB protein may be fused, operably coupled to, or otherwise associated with one or more heterologous functionals domains. In certain example embodiments, the TnpB protein may be a catalytically dead TnpB protein and/or have nickase activity. A nickase is an TnpB protein that cuts only one strand of a double stranded target. In such embodiments, the catalytically inactive TnpB or nickase provide a sequence specific targeting functionality via the ωRNA that delivers the functional domain to or proximate a target sequence.

It is also envisaged that the TnpB complex as a whole may be associated with two or more functional domains. For example, there may be two or more functional domains associated with the TnpB polypeptide, or there may be two or more functional domains associated with the nucleic acid component (via one or more adaptor proteins or aptamers), or there may be one or more functional domains associated with the TnpB polypeptide and one or more functional domains associated with the nucleic acid component.

In one embodiment, one or more functional domains are associated with a TnpB polypeptide via an adaptor protein, for example as used with the modified guides of Konnerman et al. (Nature 517, 583-588, 29 Jan. 2015). In one embodiment, the one or more functional domains is attached to the adaptor protein so that upon binding of the TnpB polypeptide to the RNA molecule and target, the functional domain is in a spatial orientation allowing for the functional domain to function in its attributed function.

In one embodiment, one or more functional domains are associated with a dead nucleic acid component. In one embodiment, a complex with active TnpB polypeptide directs gene regulation by a functional domain at on gene locus while a functional domain associated with the nucleic acid component directs DNA cleavage by the active TnpB polypeptide at another. In one embodiment, nucleic acid components are selected to maximize selectivity of regulation for a gene locus of interest compared to off-target regulation. In one embodiment, nucleic acid components are selected to maximize target gene regulation and minimize target cleavage. Loops of the nucleic acid component may be extended, without colliding with the TnpB polypeptide by the insertion of distinct loop(s) or distinct sequence(s) that may recruit adaptor proteins that can bind to the distinct loop(s) or distinct sequence(s). The adaptor proteins may include but are not limited to orthogonal polynucleotide-binding protein/aptamer combinations that exist within the diversity of bacteriophage coat proteins. A list of such coat proteins includes, but is not limited to: Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s and PRR1. These adaptor proteins or orthogonal RNA binding proteins can further recruit effector proteins or fusions which comprise one or more functional domains.

Example functional domains that may be fused to, operably coupled to, or otherwise associated with an TnpB protein can be or include, but are not limited to a nuclear localization signal (NLS) domain, a nuclear export signal (NES) domain, a translational activation domain, a transcriptional activation domain (e.g., VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, a ligase domain, a topoisomerase domain, an integrase domain, and combinations thereof. In an embodiment, the functional domain is an HNH domain, and may be used with a naturally catalytically inactive TnpB protein to engineer a nickase. Methods for generating catalytically dead TnpB or a nickase TnpB can be adapted from approaches in Cas9 proteins, see, for example, WO 2014/204725, Ran et al. Cell. 2013 Sep. 12; 154(6): 1380-1389, known in the art and incorporated herein by reference. Briefly, one or more mutations in the catalytic domain of the RuvC domain and/or the HNH domain of the TnpB protein can be introduced that may reduce or abolish NHEJ activity. In an aspect, at least one mutation in the RuvC domain and at least one mutation in the HNH domain is provided. In an embodiment, the TnpB polypeptide comprises a mutation at D191 and/or E278 based on amino acid sequence positions of Deinococcus radiodurans ISDra2. In an aspect, the amino acid mutations comprise D191A and/or E278A based on amino acid sequence positions of Deinococcus radiodurans ISDra2.

In one embodiment, the functional domains can have one or more of the following activities: nucleobase deaminse activity, reverse transcriptase activity, retrotransposase activity, transposase activity, integrase activity, recombinase activity, topoisomerase activity, ligase activity, polymerase activity, helicase activity, methylase activity, demethylase activity, translation activation activity, translation initiation activity, translation repression activity, transcription activation activity, transcription repression activity, transcription release factor activity, histone modification activity, nuclease activity (e.g., VirD2), single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In one embodiment, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a TnpB protein). In embodiments having two or more functional domains, each of the two can be positioned at or near or in proximity to a terminus of the effector protein (e.g., a TnpB protein). In one embodiment, such as those where the functional domain is operably coupled to the effector protein, the one or more functional domains can be tethered or linked via a suitable linker (including, but not limited to, GlySer linkers) to the effector protein (e.g., a TnpB protein). When there is more than one functional domain, the functional domains can be same or different. In one embodiment, all the functional domains are the same. In one embodiment, all of the functional domains are different from each other. In one embodiment, at least two of the functional domains are different from each other. In one embodiment, at least two of the functional domains are the same as each other.

Histone modifying domains are also preferred in one embodiment. Exemplary histone modifying domains are discussed below. Transposase domains, HR (Homologous Recombination) machinery domains, recombinase domains, and/or integrase domains are also preferred as the present functional domains. In one embodiment, DNA integration activity includes HR machinery domains, integrase domains, recombinase domains and/or transposase domains.

In one embodiment, the DNA cleavage activity is due to a nuclease. In one embodiment, the nuclease comprises a Fok1 nuclease. See, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77(2014), relates to dimeric RNA-guided FokI Nucleases that recognize extended sequences and can edit endogenous genes with high efficiencies in human cells.

Functional domains may be used to regulate transcription, e.g., transcriptional repression. Transcriptional repression is often mediated by chromatin modifying enzymes such as histone methyltransferases (HMTs) and deacetylases (HDACs). Repressive histone effector domains are known and an exemplary list is provided below. Proteins and functional truncations of small size to facilitate efficient viral packaging (for instance via AAV) are preferred. In general, however, the domains may include HDACs, histone methyltransferases (HMTs), and histone acetyltransferase (HAT) inhibitors, as well as HDAC and HMT recruiting proteins. The functional domain may be or include, In one embodiment, HDAC Effector Domains, HDAC Recruiter Effector Domains, Histone Methyltransferase (HMT) Effector Domains, Histone Methyltransferase (HMT) Recruiter Effector Domains, or Histone Acetyltransferase Inhibitor Effector Domains.

In one embodiment, the functional domain may be a Methyltransferase (HMT) Effector Domain. Preferred examples include NUE, vSET, EHMT2/G9A, SUV39H1, dim-5, KYP, SUVR4, SET4, SET1, SETD8, and TgSET8. NUE is exemplified in the present Examples and, although preferred, it is envisaged that others in the class will also be useful.

In one embodiment, the functional domain may be a Histone Methyltransferase (HMT) Recruiter Effector Domain. Preferred examples include Hp1a, PHF19, and NIPP1.

In one embodiment, the functional domain may be Histone Acetyltransferase Inhibitor Effector Domain. Preferred examples include SET/TAF-1B.

In some cases, the target endogenous (regulatory) control elements (such as enhancers and silencers) in addition to a promoter or promoter-proximal elements. Thus, the invention can also be used to target endogenous control elements (including enhancers and silencers) in addition to targeting of the promoter. These control elements can be located upstream and downstream of the transcriptional start site (TSS), starting from 200 bp from the TSS to 100 kb away. Targeting of known control elements can be used to activate or repress the gene of interest. In some cases, a single control element can influence the transcription of multiple target genes. Targeting of a single control element could therefore be used to control the transcription of multiple genes simultaneously.

Targeting of putative control elements on the other hand (e.g., by tiling the region of the putative control element as well as 200 bp up to 100 kB around the element) can be used as a means to verify such elements (by measuring the transcription of the gene of interest) or to detect novel control elements (e.g., by tiling 100 kb upstream and downstream of the TSS of the gene of interest). In addition, targeting of putative control elements can be useful in the context of understanding genetic causes of disease. Many mutations and common SNP variants associated with disease phenotypes are located outside coding regions. Targeting of such regions with either the activation or repression systems described herein can be followed by readout of transcription of either a) a set of putative targets (e.g., a set of genes located in closest proximity to the control element) or b) whole-transcriptome readout by e.g., RNAseq or microarray. This would allow for the identification of likely candidate genes involved in the disease phenotype. Such candidate genes could be useful as novel drug targets.

In one embodiment the one or more functional domains comprise an acetyltransferase, preferably a histone acetyltransferase. These are useful in the field of epigenomics, for example in methods of interrogating the epigenome. Methods of interrogating the epigenome may include, for example, targeting epigenomic sequences. Targeting epigenomic sequences may include the ωRNA being directed to an epigenomic target sequence. Epigenomic target sequence may include, a promoter, silencer or an enhancer sequence.

The functional domains may be acetyltransferases domains. Examples of acetyltransferases are known but may include, In one embodiment, histone acetyltransferases. In one embodiment, the histone acetyltransferase may comprise the catalytic core of the human acetyltransferase p300 (Gerbasch & Reddy, Nature Biotech 6 Apr. 2015).

### Nuclear Localization Sequences

In one embodiment, the TnpB polypeptide is fused to one or more nuclear localization sequences (NLSs), such as about or more than about 1, 2, 3, 4, 5, 6, 7, 8, 9, 10, or more NLSs. In one embodiment, the TnpB polypeptide comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In a preferred embodiment of the invention, the TnpB polypeptide comprises at most 6 NLSs. In one embodiment, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Non-limiting examples of NLSs include an NLS sequence derived from: the NLS of the SV40 virus large T-antigen, having the amino acid sequence PKKKRKV (SEQ ID NO: 193); the NLS from nucleoplasmin (e.g., the nucleoplasmin bipartite NLS with the sequence KRPAATKKAGQAKKKK (SEQ ID NO: 194); the c-myc NLS having the amino acid sequence PAAKRVKLD (SEQ ID NO: 195) or RQRRNELKRSP (SEQ ID NO: 196); the hRNPA1 M9 NLS having the sequence NQSSNFGPMKGGNFGGRSSGPYGGGGQYFAKPRNQGGY (SEQ ID NO: 197); the sequence RMRIZFKNKGKDTAELRRRRVEVSVELRKAKKDEQILKRRNV (SEQ ID NO: 198) of the IBB domain from importin-alpha; the sequences VSRKRPRP (SEQ ID NO: 199) and PPKKARED (SEQ ID NO: 200) of the myoma T protein; the sequence PQPKKKPL (SEQ ID NO: 201) of human p53; the sequence SALIKKKKKMAP (SEQ ID NO: 202) of mouse c-abl IV; the sequences DRLRR (SEQ ID NO: 203) and PKQKKRK (SEQ ID NO: 204) of the influenza virus NS1; the sequence RKLKKKIKKL (SEQ ID NO: 205) of the Hepatitis virus delta antigen; the sequence REKKKFLKRR (SEQ ID NO: 206) of the mouse Mx1 protein; the sequence KRKGDEVDGVDEVAKKKSKK (SEQ ID NO: 207) of the human poly(ADP-ribose) polymerase; and the sequence RKCLQAGMNLEARKTKK (SEQ ID NO: 208) of the steroid hormone receptors (human) glucocorticoid. In general, the one or more NLSs are of sufficient strength to drive accumulation of the TnpB polypeptide in a detectable amount in the nucleus of a eukaryotic cell. In general, strength of nuclear localization activity may derive from the number of NLSs in the TnpB polypeptide, the particular NLS(s) used, or a combination of these factors. Detection of accumulation in the nucleus may be performed by any suitable technique. For example, a detectable marker may be fused to the TnpB polypeptide, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of complex formation (e.g., assay for DNA cleavage or mutation at the target sequence, or assay for altered gene expression activity affected by complex formation and/or TnpB polypeptide activity), as compared to a control no exposed to the TnpB polypeptide or complex, or exposed to a TnpB polypeptide lacking the one or more NLSs. In one embodiment of the herein described TnpB polypeptide protein complexes and systems the codon optimized TnpB polypeptide proteins comprise an NLS attached to the C-terminal of the protein. In one embodiment, other localization tags may be fused to the TnpB polypeptide, such as without limitation for localizing the TnpB polypeptide to particular sites in a cell, such as organelles, such as mitochondria, plastids, chloroplast, vesicles, golgi, (nuclear or cellular) membranes, ribosomes, nucleolus, ER, cytoskeleton, vacuoles, centrosome, nucleosome, granules, centrioles, etc.

In one embodiment of the invention, at least one nuclear localization signal (NLS) is attached to the nucleic acid sequences encoding the TnpB polypeptide. In preferred embodiments at least one or more C-terminal or N-terminal NLSs are attached (and hence nucleic acid molecule(s) coding for the TnpB polypeptide can include coding for NLS(s) so that the expressed product has the NLS(s) attached or connected). In a preferred embodiment a C-terminal NLS is attached for optimal expression and nuclear targeting in eukaryotic cells, preferably human cells. The invention also encompasses methods for delivering multiple nucleic acid components, wherein each nucleic acid component is specific for a different target locus of interest thereby modifying multiple target loci of interest. The nucleic acid component of the complex may comprise one or more protein-binding RNA aptamers. The one or more aptamers may be capable of binding a bacteriophage coat protein.

### Linkers

In some preferred embodiments, the functional domain is linked to a TnpB polypeptide (e.g., an active or a dead TnpB polypeptide) to target and activate epigenomic sequences such as promoters or enhancers. One or more ωRNAs directed to such promoters or enhancers may also be provided to direct the binding of the TnpB polypeptide to such promoters or enhancers.

The term “associated with” is used here in relation to the association of the functional domain to the TnpB polypeptide protein or the adaptor protein. It is used in respect of how one molecule ‘associates’ with respect to another, for example between an adaptor protein and a functional domain, or between the TnpB polypeptide protein and a functional domain. In the case of such protein-protein interactions, this association may be viewed in terms of recognition in the way an antibody recognizes an epitope. Alternatively, one protein may be associated with another protein via a fusion of the two, for instance one subunit being fused to another subunit. Fusion typically occurs by addition of the amino acid sequence of one to that of the other, for instance via splicing together of the nucleotide sequences that encode each protein or subunit. Alternatively, this may essentially be viewed as binding between two molecules or direct linkage, such as a fusion protein. In any event, the fusion protein may include a linker between the two subunits of interest (i.e., between the enzyme and the functional domain or between the adaptor protein and the functional domain). Thus, in one embodiment, the TnpB polypeptide protein or adaptor protein is associated with a functional domain by binding thereto. In other embodiments, the TnpB polypeptide or adaptor protein is associated with a functional domain because the two are fused together, optionally via an intermediate linker.

The term “linker” as used in reference to a fusion protein refers to a molecule which joins the proteins to form a fusion protein. Generally, such molecules have no specific biological activity other than to join or to preserve some minimum distance or other spatial relationship between the proteins. However, in one embodiment, the linker may be selected to influence some property of the linker and/or the fusion protein such as the folding, net charge, or hydrophobicity of the linker.

Suitable linkers for use in the methods of the present invention are well known to those of skill in the art and include, but are not limited to, straight or branched-chain carbon linkers, heterocyclic carbon linkers, or peptide linkers. However, as used herein the linker may also be a covalent bond (carbon-carbon bond or carbon-heteroatom bond). In particular embodiments, the linker is used to separate the TnpB polypeptide and the nucleotide deaminase by a distance sufficient to ensure that each protein retains its required functional property. Preferred peptide linker sequences adopt a flexible extended conformation and do not exhibit a propensity for developing an ordered secondary structure. In one embodiment, the linker can be a chemical moiety which can be monomeric, dimeric, multimeric or polymeric. Preferably, the linker comprises amino acids. Typical amino acids in flexible linkers include Gly, Asn and Ser. Accordingly, in particular embodiments, the linker comprises a combination of one or more of Gly, Asn and Ser amino acids. Other near neutral amino acids, such as Thr and Ala, also may be used in the linker sequence. Exemplary linkers are disclosed in Maratea et al. (1985), Gene 40:39-46; Murphy et al. (1986) Proc. Nat'l. Acad. Sci. USA 83:8258-62; U.S. Pat. Nos. 4,935,233; and 4,751,180. For example, GlySer linkers GGS, GGGS (SEQ ID NO: 209) or GSG can be used. GGS, GSG, GGGS (SEQ ID NO: 209) or GGGGS (SEQ ID NO: 210) linkers can be used in repeats of 3 (such as (GGS)₃ (SEQ ID NO: 211), (GGGGS)₃ (SEQ ID NO: 212)) or 5, 6, 7, 9 or even 12 or more, to provide suitable lengths. In some cases, the linker may be (GGGGS)₃-15 (SEQ ID NO: 212-224), For example, in some cases, the linker may be (GGGGS)₃-11 (SEQ ID NO: 212-220), e.g., GGGGS (SEQ ID NO: 210), (GGGGS)₂ (SEQ ID NO: 225), (GGGGS)₃ (SEQ ID NO: 212), (GGGGS)₄ (SEQ ID NO: 213), (GGGGS)₅ (SEQ ID NO: 214), (GGGGS)₆ (SEQ ID NO: 215), (GGGGS)₇ (SEQ ID NO: 216), (GGGGS): (SEQ ID NO: 217), (GGGGS), (SEQ ID NO: 218), (GGGGS)₁₀ (SEQ ID NO: 219), or (GGGGS)₁₁ (SEQ ID NO: 220).

In particular embodiments, linkers such as (GGGGS)₃ (SEQ ID NO: 212) are preferably used herein. (GGGGS)₆ (SEQ ID NO: 215), (GGGGS)₉ (SEQ ID NO: 218) or (GGGGS)₁₂ (SEQ ID NO: 221) may preferably be used as alternatives. Other preferred alternatives are (GGGGS)₁ (SEQ ID NO: 210), (GGGGS)₂ (SEQ ID NO: 225), (GGGGS)₄ (SEQ ID NO: 213), (GGGGS)₅ (SEQ ID NO: 214), (GGGGS)₇ (SEQ ID NO: 216), (GGGGS): (SEQ ID NO: 217), (GGGGS)₁₀ (SEQ ID NO: 219), or (GGGGS)₁₁ (SEQ ID NO: 220). In yet a further embodiment, LEPGEKPYKCPECGKSFSQSGALTRHQRTHTR (SEQ ID NO: 226) is used as a linker. In yet an additional embodiment, the linker is an XTEN linker. In particular embodiments, the TnpB polypeptide is linked to the deaminase protein or its catalytic domain by means of an LEPGEKPYKCPECGKSFSQSGALTRHQRTHTR (SEQ ID NO: 226) linker. In further particular embodiments, TnpB polypeptide is linked C-terminally to the N-terminus of a deaminase protein or its domain by catalytic means of an LEPGEKPYKCPECGKSFSQSGALTRHQRTHTR (SEQ ID NO: 226) linker. In addition, N- and C-terminal NLSs can also function as linker (e.g., PKKKRKVEASSPKKRKVEAS (SEQ ID NO: 227)).

Examples of linkers are shown in Table 2 below.

TABLE 2

| | |
|---|---|
| GGS | GGTGGTAGT |
| GGSx3 (9) | GGTGGTAGTGGAGGGAGCGGCGGTTCA |
| | (SEQ ID NO: 228) |
| GGSx7 (21) | ggtggaggaggctctggtggaggcggt |
| | agcggaggcggagggtcgGGTGGTAGT |
| | GGAGGGAGCGGCGGTTCA |
| | (SEQ ID NO: 229) |
| XTEN | TCGGGATCTGAGACGCCTGGGACCTCG |
| | GAATCGGCTACGCCCGAAAGT |
| | (SEQ ID NO: 230) |
| Z-EGFR_ | Gtggataacaaatttaacaaagaaatg |
| Short | tgggcggcgtgggaagaaattcgtaac |
| | ctgccgaacctgaacggctggcagatg |
| | accgcgtttattgcgagcctggtggat |
| | gatccgagccagagcgcgaacctgctg |
| | gcggaagcgaaaaaactgaacgatgcg |
| | caggcgccgaaaaccggcggtggttct |
| | ggt (SEQ ID NO: 231) |
| GSAT | Ggtggttctgccggtggctccggttct |
| | ggctccagcggtggcagctctggtgcg |
| | tccggcacgggtactgcgggtggcact |
| | ggcagcggttccggtactggctctggc |
| | (SEQ ID NO: 232) |

Linkers may be used between the ωRNA molecules and the functional domain (activator or repressor), or between the TnpB polypeptide and the functional domain. The linkers may be used to engineer appropriate amounts of “mechanical flexibility”.

In one embodiment, the one or more functional domains are controllable, e.g., inducible.

Other suitable functional domains can be found, for example, in International Application Publication No. WO 2019/018423, for example, at [0678]-[0692], incorporated herein by reference. Exemplary functional domains are further detailed elsewhere herein.

### ωRNA Molecules

The TnpB systems herein may further comprise one or more nucleic acid components, which are also referred to herein as OMEGA RNA (ωRNA). Such nucleic acid component may comprise RNA, DNA, or combinations thereof and include modified and non-canonical nucleotides as described further below. The ωRNA can comprise a reprogrammable spacer sequence and a scaffold that interacts with the TnpB polypeptide. ωRNA may form a complex (52 complex) with a TnpB polypeptide, and direct sequence-specific binding of the complex to a target sequence of a target polynucleotide. In one example embodiment, the ωRNA is a single molecule comprising a scaffold sequence and a spacer sequence. In certain example embodiments, the spacer is 5′ of the scaffold sequence. In one example embodiment, the ωRNA may further comprise a conserved nucleic acid sequence between the scaffold and spacer portions.

In embodiments, the ωRNA comprises a spacer sequence and a scaffold sequence, e.g., a conserved nucleotide sequence. In embodiments, the ωRNA comprises about 45 to about 250 nucleotides, or about 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 17, 138, 19, 140, 141, 142, 143, 144, 145, 146, 147, 148, 149, 150, 151, 152, 153, 154, 155, 156, 157, 158, 159, 160, 11, 162, 163, 164, 165, 166, 167, 168, 169, 170, 171, 172, 173, 174, 175, 176, 177, 178, 179, 180. 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 2340, 241, 242, 243, 244, 245, 246, 247, 248, 249, or 250 nucleotides.

In embodiments, the ωRNA comprises a scaffold sequence, e.g., a conserved nucleotide sequence. The scaffold sequence therefore typically comprises conserved regions, with the scaffold comprising about 30 to 200 nucleotides, about 50 to 180, about 80 to 175 nucleotides, or about 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180 or more nt. In an aspect, the Nucleic acid component scaffold comprises one conserved nucleotide sequence. In embodiments, the conserved nucleotide sequence is on or near a 5′ end of the scaffold.

The ωRNA may further comprise a spacer, which can be re-programmed to direct site-specific binding to a target sequence of a target polynucleotide. The spacer may also be referred to herein as part of the ωRNA scaffold or ωRNA, and may comprise an engineered heterologous sequence. In an embodiment the scaffold may comprise a sequence from Table 5. In an embodiment, the scaffold comprises one or more conserved sequences to the RNA conserved region in Table 5 and depicted in FIG. 2. In one embodiment, the secondary structure of the ωRNA comprises multi-hairpin regions indicated in FIG. 18D. In an aspect, the RNA species comprises the RNA conserved region+guide sequence, which is distinct from but generally related to the DR+spacer configuration of CRISPR-Cas systems.

In one embodiment, the spacer length of the ωRNA is from 10 to 50 nt. In one embodiment, the spacer length of the ωRNA is at least 10, 11, 12, 13, 14, or 15 nucleotides. In one embodiment, the spacer length is from 10 to 40 nucleotides, from 15 to 30 nt, 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer. In example embodiments, the spacer sequence is 14, 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, or 50 nt.

In one embodiment, the sequence of the ωRNA is selected to reduce the degree secondary structure within the ωRNA. In one embodiment, about or less than about 75%, 50%, 40%, 30%, 25%, 20%, 15%, 10%, 5%, 1%, or fewer of the nucleotides of the nucleic acid-targeting ωRNA component participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9(1981), 133-148). Another example of a folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

As used herein, a heterologous ωRNA is an ωRNA that is not derived from the same species as the TnpB polypeptide, or comprises a portion of the molecule, e.g., spacer, that is not derived from the same species as the TnpB polypeptide. For example, a heterologous ωRNA of a TnpB polypeptide derived from species A comprises a polynucleotide derived from a species different from species A, or an artificial polynucleotide.

In a particular embodiment, the ωRNA comprises a spacer sequence linked to a conserved nucleotide sequence, wherein the conserved nucleotide sequence may comprise one or more stem loops or optimized secondary structures. In particular embodiments, the conserved nucleotide sequence has a minimum length of 16 nts and a single stem loop. In further embodiments the conserved nucleotide sequence has a length longer than 16 nts, preferably more than 17 nts, and has more than one stem loops or optimized secondary structures. In particular embodiments, the spacer sequence may be linked to all or part of the natural conserved nucleotide sequence. In particular embodiments, certain aspects of the ωRNA architecture can be modified, for example by addition, subtraction, or substitution of features, whereas certain other aspects of architecture are maintained. Preferred locations for engineered ωRNA modifications, including but not limited to insertions, deletions, and substitutions include ωRNA termini and regions of the ωRNA that are exposed when complexed with TnpB polypeptide and/or target.

In one embodiment, the ωRNA forms a stemloop with a separate non-covalently linked sequence, which can be DNA or RNA. In particular embodiments, the sequences forming the ωRNA are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In one embodiment, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the conserved nucleotide sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In one embodiment, these stem-loop forming sequences can be chemically synthesized. In one embodiment, the chemical synthesis uses automated, solid-phase oligonucleotide synthesis machines with 2′-acetoxyethyl orthoester (2′-ACE) (Scaringe et al., J. Am. Chem. Soc. (1998) 120:11820-11821; Scaringe, Methods Enzymol. (2000) 317:3-18) or 2′-thionocarbamate (2′-TC) chemistry (Dellinger et al., J. Am. Chem. Soc. (2011) 133:11540-11546; Hendel et al., Nat. Biotechnol. (2015) 33:985-989).

The repeat: anti repeat duplex will be apparent from the secondary structure of the ωRNA component. It may be typically a first complimentary stretch after (in 5′ to 3′ direction) the poly U tract and before the tetraloop; and a second complimentary stretch after (in 5′ to 3′ direction) the tetraloop and before the poly A tract. The first complimentary stretch (the “repeat”) is complimentary to the second complimentary stretch (the “anti-repeat”). As such, they Watson-Crick base pair to form a duplex of dsRNA when folded back on one another. As such, the anti-repeat sequence is the complimentary sequence of the repeat and in terms to A-U or C-G base pairing, but also in terms of the fact that the anti-repeat is in the reverse orientation due to the tetraloop.

In an embodiment of the invention, modification of the ωRNA component molecule architecture comprises replacing bases in stemloop 2. For example, in one embodiment, “actt” (“acuu” in RNA) and “aagt” (“aagu” in RNA) bases in stemloop2 are replaced with “cgcc” and “gcgg”. In one embodiment, “actt” and “aagt” bases in stemloop2 are replaced with complimentary GC-rich regions of 4 nucleotides. In one embodiment, the complimentary GC-rich regions of 4 nucleotides are “cgcc” and “gcgg” (both in 5′ to 3′ direction). In one embodiment, the complimentary GC-rich regions of 4 nucleotides are “gcgg” and “cgcc” (both in 5′ to 3′ direction). Other combination of C and G in the complimentary GC-rich regions of 4 nucleotides will be apparent including CCCC and GGGG.

In one aspect, the stemloop 2, e.g., “ACTTgtttAAGT (SEQ ID NO: 233)” can be replaced by any “XXXXgtttYYYY”, e.g., where XXXX and YYYY represent any complementary sets of nucleotides that together will base pair to each other to create a stem.

As used herein, the term “spacer” may also be referred to as a “guide sequence.” In one embodiment, the degree of complementarity of the spacer sequence to a given target sequence, when optimally aligned using a suitable alignment algorithm, is about or more than 50%, 60%, 75%, 80%, 85%, 90%, 95%, 97.5%, 99%, or more. In certain example embodiments, the ωRNA molecule comprises a spacer sequence that may be designed to have at least one mismatch with the target sequence, such that a RNA duplex formed between the sequence and the target sequence. Accordingly, the degree of complementarity is less than 99%. For instance, where the spacer sequence consists of 24 nucleotides, the degree of complementarity is more particularly about 96% or less. In particular embodiments, the spacer sequence is designed to have a stretch of two or more adjacent mismatching nucleotides, such that the degree of complementarity over the entire sequence is further reduced. For instance, where the spacer sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In one embodiment, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). The ability of a sequence (within a nucleic acid-targeting ωRNA t molecule) to direct sequence-specific binding of a nucleic acid-targeting complex to a target nucleic acid sequence may be assessed by any suitable assay. For example, the components of a ωRNA system sufficient to form a TnpB-targeting complex, including the ωRNA molecule sequence to be tested, may be provided to a host cell having the corresponding target nucleic acid sequence, such as by transfection with vectors encoding the components of the TnpB-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a TnpB-targeting complex, including the sequence to be tested and a control sequence different from the test ωRNA, and comparing binding or rate of cleavage at or in the vicinity of the target sequence between the test and control ωRNA molecule sequence reactions. Other assays are possible, and will occur to those skilled in the art. A spacer sequence, and hence a nucleic acid-targeting ωRNA may be selected to target any target nucleic acid sequence.

A ωRNA, and hence a nucleic acid-targeting spacer, may be selected to target any target nucleic acid sequence. The target sequence may be DNA. The target sequence may be any RNA sequence. In one embodiment, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In one embodiment, the ωRNA forms a stemloop with a separate non-covalently linked sequence, which can be DNA or RNA. In particular embodiments, the sequences forming the ωRNA component are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In one embodiment, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the conserved nucleotide sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

### ωRNA Chemical Modifications

In one embodiment, these stem-loop forming sequences can be chemically synthesized. In one embodiment, the chemical synthesis uses automated, solid-phase oligonucleotide synthesis machines with 2′-acetoxyethyl orthoester (2′-ACE) (Scaringe et al., J. Am. Chem. Soc. (1998) 120:11820-11821; Scaringe, Methods Enzymol. (2000) 317:3-18) or 2′-thionocarbamate (2′-TC) chemistry (Dellinger et al., J. Am. Chem. Soc. (2011) 133:11540-11546; Hendel et al., Nat. Biotechnol. (2015) 33:985-989).

In one embodiment, the ωRNA component molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the ωRNA sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a ωRNA component nucleic acid comprises ribonucleotides and non-ribonucleotides. In one such embodiment, a ωRNA component comprises one or more ribonucleotides and one or more deoxyribonucleotides. In an embodiment of the invention, the ωRNA component comprises one or more non-naturally occurring nucleotide or nucleotide analog such as a nucleotide with phosphorothioate linkage, a locked nucleic acid (LNA) nucleotide comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of ωRNA chemical modifications include, without limitation, incorporation of 2′-O-methyl (M), 2′-O-methyl 3′phosphorothioate (MS), S-constrained ethyl (cEt), or 2′-O-methyl 3′thioPACE (MSP) at one or more terminal nucleotides. Such chemically modified ωRNA components can comprise increased stability and increased activity as compared to unmodified ωRNA components, though on-target vs. off-target specificity is not predictable. (See, Hendel, 2015, Nat Biotechnol. 33(9): 985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., Med Chem Comm., 2014, 5:1454-1471; Hendel et al., Nat. Biotechnol. (2015) 33(9): 985-989; Li et al., Nature Biomedical Engineering, 2017, 1, 0066 DOI: 10.1038/s41551-017-0066). In one embodiment, the 5′ and/or 3′ end of a ωRNA component is modified by a variety of functional moieties including fluorescent dyes, polyethylene glycol, cholesterol, proteins, or detection tags. (See Kelly et al., 2016, J. Biotech. 233:74-83). In one embodiment, a ωRNA component comprises ribonucleotides in a region that binds to a target sequence and one or more deoxyribonucletides and/or nucleotide analogs in a region that binds to the TnpB polypeptide. In an embodiment, deoxyribonucleotides and/or nucleotide analogs are incorporated in engineered ωRNA component structures. In one embodiment, 3-5 nucleotides at either the 3′ or the 5′ end of a ωRNA component is chemically modified. In one embodiment, only minor modifications are introduced in the seed region, such as 2′-F modifications. In one embodiment, 2′-F modification is introduced at the 3′ end of a ωRNA component. In one embodiment, three to five nucleotides at the 5′ and/or the 3′ end of the ωRNA component are chemically modified with 2′-O-methyl (M), 2′-O-methyl 3′ phosphorothioate (MS), S-constrained ethyl (cEt), or 2′-O-methyl 3′ thioPACE (MSP). Such modification can enhance genome editing efficiency (see Hendel et al., Nat. Biotechnol. (2015) 33(9): 985-989). In one embodiment, all of the phosphodiester bonds of a ωRNA component are substituted with phosphorothioates (PS) for enhancing levels of gene disruption. In one embodiment, more than five nucleotides at the 5′ and/or the 3′ end of the ωRNA component are chemically modified with 2′-O-Me, 2′-F or S-constrained ethyl (cEt). Such chemically modified ωRNA component can mediate enhanced levels of gene disruption (see Ragdarm et al., 0215, PNAS, E7110-E7111). In an embodiment of the invention, a ωRNA component is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the ωRNA component by a linker, such as an alkyl chain. In one embodiment, the chemical moiety of the modified Nucleic acid component can be used to attach the ωRNA component to another molecule, such as DNA, RNA, protein, or nanoparticles. Such chemically modified ωRNA component can be used to identify or enrich cells generically edited by a TnpB polypeptide and related systems (see Lee et al., eLife, 2017, 6: e25312, DOI: 10.7554).

In a particular embodiment, the conserved nucleotide sequence may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In embodiments, the TnpB polypeptide utilizes the ωRNA component scaffold comprising a polynucleotide sequence that facilitates the interaction with the TnpB protein, allowing for sequence specific binding and/or targeting of the Nucleic acid component molecule with the target polynucleotide. Chemical synthesis of the ωRNA component scaffold is contemplated, using covalent linkage using various bioconjugation reactions, loops, bridges, and non-nucleotide links via modifications of sugar, internucleotide phosphodiester bonds, purine and pyrimidine residues. Sletten et al., Angew. Chem. Int. Ed. (2009) 48:6974-6998; Manoharan, M. Curr. Opin. Chem. Biol. (2004) 8:570-9; Behlke et al., Oligonucleotides (2008) 18:305-19; Watts, et al., Drug. Discov. Today (2008) 13:842-55; Shukla, et al., Chem Med Chem (2010) 5:328-49; chemical synthesis using automated, solid-phase oligonucleotide synthesis machines with 2′-acetoxyethyl orthoester (2′-ACE) (Scaringe et al., J. Am. Chem. Soc. (1998) 120:11820-11821; Scaringe, Methods Enzymol. (2000) 317:3-18) or 2′-thionocarbamate (2′-TC) chemistry (Dellinger et al., J. Am. Chem. Soc. (2011) 133:11540-11546; Hendel et al., Nat. Biotechnol. (2015) 33:985-989).

In certain example embodiments, the scaffold and spacer may be designed as two separate molecules that can hybridize or covalently joined into a single molecule. Covalent linkage can be via a linker (e.g., a non-nucleotide loop) that comprises a moiety such as spacers, attachments, bioconjugates, chromophores, reporter groups, dye labeled RNAs, and non-naturally occurring nucleotide analogues. More specifically, suitable spacers for purposes of this invention include, but are not limited to, polyethers (e.g., polyethylene glycols, polyalcohols, polypropylene glycol or mixtures of efhylene and propylene glycols), polyamines group (e.g., spennine, spermidine and polymeric derivatives thereof), polyesters (e.g., poly(ethyl acrylate)), polyphosphodiesters, alkylenes, and combinations thereof. Suitable attachments include any moiety that can be added to the linker to add additional properties to the linker, such as but not limited to, fluorescent labels. Suitable bioconjugates include, but are not limited to, peptides, glycosides, lipids, cholesterol, phospholipids, diacyl glycerols and dialkyl glycerols, fatty acids, hydrocarbons, enzyme substrates, steroids, biotin, digoxigenin, carbohydrates, polysaccharides. Suitable chromophores, reporter groups, and dye-labeled RNAs include, but are not limited to, fluorescent dyes such as fluorescein and rhodamine, chemiluminescent, electrochemiluminescent, and bioluminescent marker compounds. The design of example linkers conjugating two nucleic acid components which can be adapted for use with ωRNAs are also described in WO 2004/015075.

The linker (e.g., a non-nucleotide loop) can be of any length. In one embodiment, the linker has a length equivalent to about 0-16 nucleotides. In one embodiment, the linker has a length equivalent to about 0-8 nucleotides. In one embodiment, the linker has a length equivalent to about 0-4 nucleotides. In one embodiment, the linker has a length equivalent to about 2 nucleotides. Example linker design is also described in International Patent Publication No. WO 2011/008730.

### Escorted ωRNA Components

In particular embodiments, the compositions or complexes have a ωRNA component molecule with a functional structure designed to improve ωRNA component molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7(2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8(2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green fluorescent protein (Paige, Jeremy S., Karen Y. Wu, and Samie R. Jaffrey. “RNA mimics of green fluorescent protein.” Science 333.6042(2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1(2010): 4).

Accordingly, in particular embodiments, the ωRNA component molecule is modified, e.g., by one or more aptamer(s) designed to improve ωRNA component molecule delivery, including delivery across the cellular membrane, to intracellular compartments, or into the nucleus. Such a structure can include, either in addition to the one or more aptamer(s) or without such one or more aptamer(s), moiety(ies) so as to render the Nucleic acid component molecule deliverable, inducible or responsive to a selected effector. The invention accordingly comprehends a ωRNA component molecule that responds to normal or pathological physiological conditions, including without limitation pH, hypoxia, 02 concentration, temperature, protein concentration, enzymatic concentration, lipid structure, light exposure, mechanical disruption (e.g., ultrasound waves), magnetic fields, electric fields, or electromagnetic radiation.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

Energy sources such as electromagnetic radiation, sound energy or thermal energy may induce the Nucleic acid component molecule. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm2. In a preferred embodiment, a stimulation paradigm of as low as 0.25 sec every 15 sec should result in maximal activation.

The chemical or energy sensitive ωRNA component may undergo a conformational change upon induction by the binding of a chemical source or by the energy allowing it act as a ωRNA and have the TnpB polypeptide system or complex function. The invention can involve applying the chemical source or energy so as to have the ωRNA function and the TnpB polypeptide system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans; 4/164/rs2), 2. FKBP-FRB based system inducible by rapamycin (or related chemicals based on rapamycin) (see, e.g., nature.com/nmeth/journal/v2/n6/full/nmeth763.html), 3. GID1-GAI based system inducible by Gibberellin (GA) (see, e.g., nature.com/nchembio/journal/v8/n5/full/nchembio.922.html).

A chemical inducible system can be an estrogen receptor (ER) based system inducible by 4-hydroxytamoxifen (4OHT) (see, e.g., pnas.org/content/104/3/1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogen receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel-based system inducible by energy, heat or radio-wave (see, e.g., sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the nucleic acid component and the other components of the TnpB polypeptide/ωRNA molecule complex or system, and the binding will induce the change of sub-cellular localization of the polypeptide, leading to the entire polypeptide entering the nucleus of cells. Once inside the nucleus, the nucleic acid component protein, and the other components of the TnpB polypeptide/ωRNA molecule complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 us and 500 milliseconds, preferably between 1 us and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc., as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100.mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably, the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably, the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz′ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably, the exposure to an ultrasound energy source is at a power density of from about 0.05 to about 100 Wcm-2. Even more preferably, the exposure to an ultrasound energy source is at a power density of from about 1 to about 15 Wcm-2.

Preferably, the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably, the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the ωRNA molecule is modified by a secondary structure to increase the specificity of the TnpB polypeptide and related system and the secondary structure can protect against exonuclease activity and allow for 5′ additions to the nucleic acid component sequence also referred to herein as a protected nucleic acid component molecule.

In one aspect, the invention provides for hybridizing a “protector RNA” to a sequence of the nucleic acid component molecule, wherein the “protector RNA” is an RNA strand complementary to the 3′ end of the nucleic acid component molecule to thereby generate a partially double-stranded nucleic acid component. In an embodiment of the invention, protecting mismatched bases (i.e., the bases of the nucleic acid component molecule which do not form part of the nucleic acid component sequence) with a perfectly complementary protector sequence decreases the likelihood of target DNA binding to the mismatched basepairs at the 3′ end. In particular embodiments of the invention, additional sequences comprising an extended length may also be present within the nucleic acid component molecule such that the nucleic acid component comprises a protector sequence within the nucleic acid component molecule. This “protector sequence” ensures that the nucleic acid component molecule comprises a “protected sequence” in addition to an “exposed sequence” (comprising the part of the nucleic acid component sequence hybridizing to the target sequence). In particular embodiments, the nucleic acid component molecule is modified by the presence of the protector nucleic acid component to comprise a secondary structure such as a hairpin. Advantageously there are three or four to thirty or more, e.g., about 10 or more, contiguous base pairs having complementarity to the protected sequence, the nucleic acid component sequence or both. It is advantageous that the protected portion does not impede thermodynamics of the TnpB polypeptide and related system interacting with its target. By providing such an extension including a partially double stranded nucleic acid component molecule, the nucleic acid component molecule is considered protected and results in improved specific binding of the TnpB polypeptide/nucleic acid component molecule complex, while maintaining specific activity.

In particular embodiments, use is made of a truncated ωRNA component (tru-nucleic acid component), i.e., a nucleic acid component molecule which comprises a nucleic acid component sequence which is truncated in length with respect to the canonical nucleic acid component sequence length. As described by Nowak et al. (Nucleic Acids Res (2016) 44(20): 9555-9564), such nucleic acid component molecules may allow catalytically active TnpB polypeptide to bind its target without cleaving the target DNA. In particular embodiments, a truncated nucleic acid component is used which allows the binding of the target but retains only nickase activity of the TnpB polypeptide.

In one embodiment, conjugation of triantennary N-acetyl galactosamine (GalNAc) to oligonucleotide components may be used to improve delivery, for example delivery to select cell types, for example hepatocytes (see International Patent Publication No. WO 2014/118272 incorporated herein by reference; Nair, J K et al., 2014, Journal of the American Chemical Society 136(49), 16958-16961). This is considered to be a sugar-based particle and further details on other particle delivery systems and/or formulations are provided herein. GalNAc can therefore be considered to be a particle in the sense of the other particles described herein, such that general uses and other considerations, for instance delivery of said particles, apply to GalNAc particles as well. A solution-phase conjugation strategy may for example be used to attach triantennary GalNAc clusters (mol. wt. ˜2000) activated as PFP (pentafluorophenyl) esters onto 5′-hexylamino modified oligonucleotides (5′-HA ASOs, mol. wt. ˜8000 Da; Østergaard et al., Bioconjugate Chem., 2015, 26(8), pp 1451-1455). Similarly, poly(acrylate) polymers have been described for in vivo nucleic acid delivery (see WO2013158141 incorporated herein by reference). In further alternative embodiments, pre-mixing TnpB polypeptide nanoparticles (or protein complexes) with naturally occurring serum proteins may be used in order to improve delivery (Akinc A et al, 2010, Molecular Therapy vol. 18 no. 7, 1357-1364).

Screening techniques are available to identify delivery enhancers, for example by screening chemical libraries (Gilleron J. et al., 2015, Nucl. Acids Res. 43(16): 7984-8001). Approaches have also been described for assessing the efficiency of delivery vehicles, such as lipid nanoparticles, which may be employed to identify effective delivery vehicles for components (see Sahay G. et al., 2013, Nature Biotechnology 31, 653-658).

### Target Adjacent Motifs

The TnpB systems disclosed herein may recognize a target adjacent motif (TAM) in order to recognize and bind a target sequence on a target polynucleotide. In one embodiment, the nucleic acid-guided nucleases and related compositions do not contain a TAM requirement. The precise sequence and length requirements for the TAM will differ depending on the nucleic acid-guided nucleases used. In some examples, TAMs are typically 2-5 base pair sequences adjacent the protospacer (that is, the target sequence). In one example embodiment, the TAM is 3′ adjacent to the target polynucleotide. In another example embodiment, the TAM is 5′ adjacent to the target sequence of the target polynucleotide.

In one embodiment, the cleavage site is distant from the TAM, e.g., the cleavage occurs after the nth nucleotide on the non-target strand and after the nucleotide on the targeted strand. In one embodiment, the cleavage site occurs after an identified nucleotide (counted from the TAM) on the non-target strand and after the further identified nucleotide (counted from the TAM) on the targeted strand. In one embodiment, a vector encodes a nucleic acid-targeting effector protein that may be mutated with respect to a corresponding wild-type enzyme such that the mutated nucleic acid-targeting effector protein lacks the ability to cleave one or both DNA and RNA strands of a target polynucleotide containing a target sequence.

In one example embodiment the TAM sequence is TCAG. In another example embodiment, the TAM sequence is TCAA. In another example embodiment, the TAM sequence is TTCAN. TAM identification and specificity may be identified, for example, using the methods disclosed in the Examples section below.

### HDR Donor Templates

In one embodiment, the compositions and systems herein may further comprise one or more HDR donor templates for use in homology-directed repair mediated editing. In some cases, the HDR donor template may comprise one or more polynucleotides. In certain cases, the HDR donor template may comprise coding sequences for one or more polynucleotides. The HDR donor template may be a DNA template.

The HDR donor template may be used for editing the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the HDR donor template alters a stop codon in the target polynucleotide. For example, the HDR donor template may correct a premature stop codon. The correction may be achieved by deleting the stop codon or introduces one or more mutations to the stop codon. In other example embodiments, the HDR donor template addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g., sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of a the corresponding wild-type gene. In certain example embodiments, these defective genes may be associated with one or more disease phenotypes. In certain example embodiments, the defective gene or gene fragment is not replaced but the systems described herein are used to insert HDR donor templates that encode gene or gene fragments that compensate for or override defective gene expression such that cell phenotypes associated with defective gene expression are eliminated or changed to a different or desired cellular phenotype.

In an embodiment of the invention, the HDR donor template may include, but not be limited to, genes or gene fragments, encoding proteins or RNA transcripts to be expressed, regulatory elements, repair templates, and the like. According to the invention, the HDR donor templates may comprise left end and right end sequence elements that function with transposition components that mediate insertion.

In certain cases, the HDR donor template manipulates a splicing site on the target polynucleotide. In some examples, the HDR donor template disrupts a splicing site. The disruption may be achieved by inserting the polynucleotide to a splicing site and/or introducing one or more mutations to the splicing site. In certain examples, the HDR donor template may restore a splicing site. For example, the polynucleotide may comprise a splicing site sequence.

The HDR donor template to be inserted may has a size from 10 basepair or nucleotides to 50 kb in length, e.g., from 50 to 40k, from 100 and 30 k, from 100 to 10000, from 100 to 300, from 200 to 400, from 300 to 500, from 400 to 600, from 500 to 700, from 600 to 800, from 700 to 900, from 800 to 1000, from 900 to from 1100, from 1000 to 1200, from 1100 to 1300, from 1200 to 1400, from 1300 to 1500, from 1400 to 1600, from 1500 to 1700, from 600 to 1800, from 1700 to 1900, from 1800 to 2000 base pairs (bp) or nucleotides in length.

### Systems and Complexes

In one aspect, the present disclosure provides nucleic acid-targeting systems. Such systems may be used to target, modify, and otherwise manipulate target polynucleotides. In one embodiment, the systems comprise the TnpB polypeptide and one or more ωRNAs. The TnpB polypeptide may have nuclease activity, e.g., capable of cleaving DNA. In some embodiments the TnpB polypeptide may, or be engineered to have nickase activity, e.g., capable of generating a single-strand break on a double-strand nucleic acid such as dsDNA or dsRNA.

In some examples, two or more of the components in a system herein may form a complex. For example, the components are separate molecules but interact with each other directly or indirectly. In certain two or more of the components in a system herein may be comprised in a fusion protein.

As used herein, “target sequence” refers to a sequence to which a ωRNA is designed to have complementarity, where hybridization between a target sequence and a ωRNA promotes the formation of a polynucleotide targeting complex. Full complementarity is not necessarily required, provided there is sufficient complementarity to cause hybridization and promote formation of a TnpB-targeting complex. A target sequence may comprise DNA polynucleotides. In one embodiment, a target sequence is located in the nucleus or cytoplasm of a cell. In one embodiment, the target sequence may be within an organelle of a eukaryotic cell, for example, mitochondrion or chloroplast. A sequence or template that may be used for recombination into the targeted locus comprising the target sequences is referred to as an “editing template” or “editing sequence”. In aspects of the invention, an exogenous template may be referred to as an editing template. In an aspect the recombination is homologous recombination.

In one embodiment, formation of a TnpB-targeting complex (comprising a ωRNA hybridized to a target sequence and complexed with one or more nucleic acid-targeting effector proteins) results in cleavage of one or both nucleic acid strands in or near (e.g., within 1, 2, 3, 4, 5, 6, 7, 8, 9, 10, 20, 50, or more base pairs from) the target sequence. In one embodiment, one or more vectors driving expression of one or more elements of the TnpB system are introduced into a host cell such that expression of the elements of the TnpB system direct formation of a TnpB complex at one or more target sites. For example, a TnpB polypeptide and a ωRNA could each be operably linked to separate regulatory elements on separate vectors. Alternatively, two or more of the elements expressed from the same or different regulatory elements, may be combined in a single vector, with one or more additional vectors providing any components of the TnpB system not included in the first vector. TnpB system elements combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In one embodiment, a single promoter drives expression of a transcript encoding a TnpB and a ωRNA embedded within one or more intron sequences (e.g., each in a different intron, two or more in at least one intron, or all in a single intron). In one embodiment, the TnpB polypeptide and ωRNAs are operably linked to and expressed from the same promoter.

The present disclosure encompasses computational methods and algorithms to predict new TnpB polypeptides, identify the components, and new TnpB systems therein. In some examples, a computational method of identifying novel TnpB polypeptide loci analysis of the candidates may be conducted by searching metagenomics databases for additional homologs.

In one aspect the identifying all predicted protein coding genes is carried out by comparing the identified genes with TnpB polypeptide specific profiles and annotating them according to NCBI Conserved Domain Database (CDD) which is a protein annotation resource that consists of a collection of well-annotated multiple sequence alignment models for ancient domains and full-length proteins. These are available as position-specific score matrices (PSSMs) for fast identification of conserved domains in protein sequences via RPS-BLAST. CDD content includes NCBI-curated domains, which use 3D-structure information to explicitly define domain boundaries and provide insights into sequence/structure/function relationships, as well as domain models imported from a number of external source databases (Pfam, SMART, COG, PRK, TIGRFAM).

In a further aspect, the case-by-case analysis is performed using PSI-BLAST (Position-Specific Iterative Basic Local Alignment Search Tool). PSI-BLAST derives a position-specific scoring matrix (PSSM) or profile from the multiple sequence alignment of sequences detected above a given score threshold using protein-protein BLAST. This PSSM is used to further search the database for new matches, and is updated for subsequent iterations with these newly detected sequences. Thus, PSI-BLAST provides a means of detecting distant relationships between proteins.

In another aspect, the case-by-case analysis is performed using HHpred, a method for sequence database searching and structure prediction that is as easy to use as BLAST or PSI-BLAST and that is at the same time much more sensitive in finding remote homologs. In fact, HHpred's sensitivity is competitive with the most powerful servers for structure prediction currently available. HHpred is the first server that is based on the pairwise comparison of profile hidden Markov models (HMMs). Whereas most conventional sequence search methods search sequence databases such as UniProt or the NR, HHpred searches alignment databases, like Pfam or SMART. This greatly simplifies the list of hits to a number of sequence families instead of a clutter of single sequences. All major publicly available profile and alignment databases are available through HHpred. HHpred accepts a single query sequence or a multiple alignment as input. Within only a few minutes it returns the search results in an easy-to-read format similar to that of PSI-BLAST. Search options include local or global alignment and scoring secondary structure similarity. HHpred can produce pairwise query-template sequence alignments, merged query-template multiple alignments (e.g., for transitive searches), as well as 3D structural models calculated by the MODELLER software from HHpred alignments.

### Specialized Systems

The TnpB polypeptide may be in a dead form, e.g., does not have nuclease or nickase activity. In one embodiment, the systems further comprising one or more functional domains, e.g., nucleotide deaminase, reverse transcriptase, non-LTR retrotransposon (and protein encoded), polymerase, diversity generating element (and protein encoded) and integrases. In some examples, the systems further comprise one or more donor polynucleotides. The donor polynucleotides may be inserted to a target polynucleotide by the systems. The donor polynucleotide may be comprised in or coded by a nucleic acid template.

### TnpB Base Editing Systems

The present disclosure also provides for base editing systems. In general, such a system may comprise a nucleobase deaminase (e.g., an adenosine deaminase or cytidine deaminase) associated (e.g., fused) with a TnpB polypeptide. The TnpB polypeptide may be a catalytically inactive, or dead TnpB polypeptide, dTnpB. In certain examples, the nucleobase deaminase is a mutated form of an adenosine deaminase. The mutated form of the adenosine deaminase may have both adenosine deaminase and cytidine deaminase activities.

In some examples, the present disclosure provides an engineered, non-naturally occurring composition comprising: a dTnpB, a nucleobase deaminase associated with or otherwise capable of forming a complex with the dTnpB, and a ωRNA capable of forming a complex with the TnpB protein and directing site-specific binding at a target sequence at or adjacent to a single nucleotide or nucleotide base pair to be edited. In an aspect, the nucleotide deaminase or other editing enzyme flips the target base within the DNA. See, e.g., Hong and Cheng et al., DNA Base Flipping: A general Mechanism for Writing Reading and Erasing DNA Modifications, Adv. Exp Med Biol., 2016:945:321-341, doi: 10.1007/978-3-316-43624-1_14. Without being bound by theory, the TnpB-ωRNA complex bound to the target provides a more open pocket relative to, for example, a CRISPR-Cas protein, e.g., Cas9, Cas12, which advantageously provides more accessibility for the complex and the base flipping of the target nucleotide by the deaminase or other base editing enzyme, reducing steric hindrance and making it possible to enhance the specificity of the base editing system.

In an aspect, the bae edits can be targeted from about 2 to 100 base pairs from the end of the TAM, or about 4 to 100, 50 to 100, 6, to 100, 7 to 100, 8 to 100, 9. To 100, 10, to 100, 11 to 100, 12 to 100, 13 to 100, 14 to 100, 15 to 100, 16 to 100, 17 to 100, 18 to 100, 19 to 100, 20 to 100, 25 to 100, 3 to 90, 3 to 80, 3 to 70, 3 to 60, 3 to 50, 3 to 40, 3 to 30, or about 3 to 30 base pairs from the end of the TAM. In an aspect, when the base editor is fused to the TnpB, the linker length can be configured to allow for more precise base editing at the desired location. For example, the linker length, as detailed elsewhere herein, can be tuned to facilitate base editing closer or more distant to the TAM, and may be configured with increasing or decreasing rigidity and other properties to generate a desired configuration or presentation at the binding site. A more open configuration at the binding pocket of the TnpB complex may allow more flexibility in configuration of the TnpB editing system and specificity in access to target sites.

In one aspect, the present disclosure provides an engineered adenosine deaminase. The engineered adenosine deaminase may comprise one or more mutations herein. In one embodiment, the engineered adenosine deaminase has cytidine deaminase activity. In certain examples, the engineered adenosine deaminase has both cytidine deaminase activity and adenosine deaminase. In some cases, the modifications by base editors herein may be used for targeting post-translational signaling or catalysis. In one embodiment, compositions herein comprise nucleotide sequence comprising encoding sequences for one or more components of a base editing system. A base-editing system may comprise a deaminase (e.g., an adenosine deaminase or cytidine deaminase) fused with a TnpB polypeptide or a variant thereof. In some cases, the target polynucleotide is edited at one or more bases to introduce a G→A or C→T mutation.

In some cases, the adenosine deaminase is double-stranded RNA-specific adenosine deaminase (ADAR). Examples of ADARs include those described Yiannis A Savva et al., The ADAR protein family, Genome Biol. 2012; 13(12): 252, which is incorporated by reference in its entirety. In some examples, the ADAR may be hADAR1. In certain examples, the ADAR may be hADAR2. The sequence of hADAR2 may be that described under Accession No. AF525422.1.

In some cases, the deaminase may be a deaminase domain, e.g., a deaminase domain of ADAR (“ADAR-D”). In one example, the deaminase may be the deaminase domain of hADAR2 (“hADAR2-D), e.g., as described in Phelps K J et al., Recognition of duplex RNA by the deaminase domain of the RNA editing enzyme ADAR2. Nucleic Acids Res. 2015 January; 43(2): 1123-32, which is incorporated by reference herein in its entirety. In a particular example, the hADAR2-D has a sequence comprising amino acid 299-701 of hADAR2-D, e.g., amino acid 299-701 of the sequence under Accession No. AF525422.1.

In certain examples, the system comprises a mutated form of an adenosine deaminase fused with a dTnpB. The mutated form of the adenosine deaminase may have both adenosine deaminase and cytidine deaminase activities. In one embodiment, the adenosine deaminase may comprise one or more of the mutations: E488Q based on amino acid sequence positions of hADAR2-D, and mutations in a homologous ADAR protein corresponding to the above. In one embodiment, the adenosine deaminase may comprise one or more of the mutations: E488Q, V351G, based on amino acid sequence positions of hADAR2-D, and mutations in a homologous ADAR protein corresponding to the above. In one embodiment, the adenosine deaminase may comprise one or more of the mutations: E488Q, V351G, S486A, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, 1398V, K350I, M383L, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, 1398V, K350I, M383L, D619G, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, D619G, S582T, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, 1398V, K350I, M383L, D619G, S582T, V440I based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, 1398V, K350I, M383L, D619G, S582T, V440I, S495N based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, 1398V, K350I, M383L, D619G, S582T, V440I, S495N, K418E based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, 1398V, K350I, M383L, D619G, S582T, V440I, S495N, K418E, S661T based on amino acid sequence positions of hADAR2-D, and mutations in a homologous ADAR protein corresponding to the above. In some examples, provided herein includes a mutated adenosine deaminase e.g., an adenosine deaminase comprising one or more mutations of E488Q, V351G, S486A, T375S, S370C, P462A, N597I, L332I, I398V, K350I, M383L, D619G, S582T, V440I, S495N, K418E, S661T, fused with a dead TnpB polypeptide or TnpB polypeptide nickase. In some examples, provided herein includes a mutated adenosine deaminase e.g., an adenosine deaminase comprising E488Q, V351G, S486A, T375S, S370C, P462A, N597I, L332I, 1398V, K350I, M383L, D619G, S582T, V440I, S495N, K418E, and S661T, fused with a dead TnpB polypeptide or TnpB polypeptide nickase. In some examples, provided herein includes a mutated adenosine deaminase e.g., an adenosine deaminase comprising E488Q, V351G, S486A, T375S, S370C, P462A, N597I, L332I, 1398V, K350I, M383L, D619G, S582T, V440I, S495N, K418E, S661T, and S375N fused with a dead TnpB polypeptide or TnpB polypeptide nickase.

In one embodiment, the adenosine deaminase may be a tRNA-specific adenosine deaminase or a variant thereof. In one embodiment, the adenosine deaminase may comprise one or more of the mutations: W23L, W23R, R26G, H36L, N37S, P48S, P48T, P48A, I49V, R51L, N72D, L84F, S97C, A106V, D108N, H123Y, G125A, A142N, S146C, D147Y, R152H, R152P, E155V, I156F, K157N, K161T, based on amino acid sequence positions of E. coli TadA, and mutations in a homologous deaminase protein corresponding to the above. In one embodiment, the adenosine deaminase may comprise one or more of the mutations: D108N based on amino acid sequence positions of E. coli TadA, and mutations in a homologous deaminase protein corresponding to the above. In one embodiment, the adenosine deaminase may comprise one or more of the mutations: A106V, D108N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, based on amino acid sequence positions of E. coli TadA, and mutations in a homologous deaminase protein corresponding to the above. In one embodiment, the adenosine deaminase may comprise one or more of the mutations: A106V, D108N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, 1156F, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, 1156F, A142N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, A142N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, A142N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, R152P, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, R152P, A142N, based on amino acid sequence positions of E. coli TadA, and mutations in a homologous deaminase protein corresponding to the above.

In some examples, the base editing systems may comprise an intein-mediated trans-splicing system that enables in vivo delivery of a base editor, e.g., a split-intein cytidine base editors (CBE) or adenine base editor (ABE) engineered to trans-splice. Examples of such base editing systems include those described in Colin K. W. Lim et al., Treatment of a Mouse Model of ALS by In Vivo Base Editing, Mol Ther. 2020 Jan. 14. pii: S1525-0016(20) 30011-3. doi: 10.1016/j.ymthe.2020.01.005; and Jonathan M. Levy et al., Cytosine and adenine base editing of the brain, liver, retina, heart and skeletal muscle of mice via adeno-associated viruses, Nature Biomedical Engineering volume 4, pages 97-110(2020), which are incorporated by reference herein in their entireties.

Examples of base editing systems include those described in International Patent Publication Nos. WO 2019/071048 (e.g., paragraphs [0933]-[0938]), WO 2019/084063 (e.g., paragraphs [0173]-[0186], [0323]-[0475], [0893]-[1094]), WO 2019/126716 (e.g., paragraphs [0290]-[0425], [1077]-[1084]), WO 2019/126709 (e.g., paragraphs [0294]-[0453]), WO 2019/126762 (e.g., paragraphs [0309]-[0438]), WO 2019/126774 (e.g., paragraphs [0511]-[0670]), Cox DBT, et al., RNA editing with CRISPR-Cas13, Science. 2017 Nov. 24; 358(6366): 1019-1027; Abudayyeh O O, et al., A cytosine deaminase for programmable single-base RNA editing, Science 26 Jul. 2019: Vol. 365, Issue 6451, pp. 382-386; Gaudelli N M et al., Programmable base editing of A.T to G.C in genomic DNA without DNA cleavage, Nature volume 551, pages 464-471(23 Nov. 2017); Komor A C, et al., Programmable editing of a target base in genomic DNA without double-stranded DNA cleavage. Nature. 2016 May 19; 533(7603): 420-4; Jordan L. Doman et al., Evaluation and minimization of Cas9-independent off-target DNA editing by cytosine base editors, Nat Biotechnol (2020). doi.org/10.1038/s41587-020-0414-6; and Richter M F et al., Phage-assisted evolution of an adenine base editor with improved Cas domain compatibility and activity, Nat Biotechnol (2020). doi.org/10.1038/s41587-020-0453-z, which are incorporated by reference herein in their entireties and can be used to adapt to the TnpB polypeptides.

### TnpB Prime Editing Systems

In one embodiment, the present disclosure provides compositions and systems may comprise a TnpB or a dTnpB, one or more nucleic acid components, and a reverse transcriptase. The systems may be used to insert a donor polynucleotide to a target polynucleotide. In some examples, the composition or system comprises a catalytically inactive TnpB polypeptide, a reverse transcriptase associated with or otherwise capable of forming a complex with the TnpB polypeptide, and a nucleic acid component molecule capable of forming a complex with the TnpB polypeptide and directing site-specific binding of the complex to a target sequence of a target polynucleotide, the nucleic acid component molecule further comprising a donor template which functions as a template for insertion of a donor sequence into a target polynucleotide by the reverse transcriptase.

In some cases, the TnpB or dTnpB may be a nickase, e.g., a DNA nickase. The TnpB nickase may comprise or more mutations. In some examples, the TnpB comprises mutations corresponding to the mutations in the RuvC nuclease. In some examples, the TnpB is naturally catalytically inactive and comprises fusion to a nuclease domain, e.g., HNH or FokI domain.

A reverse transcriptase domain may be a reverse transcriptase or a fragment thereof. In certain aspects, the reverse transcriptase is Human immunodeficiency virus (HIV) RT, Avian myoblastosis virus (AMV) RT, Moloney murine leukemia virus (M-MLV) RT a group II intron RT, a group II intron-like RT, or a chimeric RT. In one embodiment, the RT comprises modified forms of these RTs, such as, engineered variants of Avian myoblastosis virus (AMV) RT, Moloney murine leukemia virus (M-MLV) RT, or Human immunodeficiency virus (HIV) RT (see, e.g., Anzalone, et al., Search-and-replace genome editing without double-strand breaks or donor DNA, Nature. 2019 December; 576(7785): 149-157).

In some examples, the compositions and systems may comprise the TnpB protein herein; a reverse transcriptase (RT) polypeptide connected to or otherwise capable of forming a complex with the TnpB protein; and a ωRNA molecule capable of forming a complex with the TnpB protein and comprising: a ωRNA sequence capable of directing site-specific binding of the TnpB complex to a target sequence of a target polynucleotide; a 3′ binding site region capable of binding to a cleaved upstream strand of the target polynucleotide; and a RT template sequence encoding an extended sequence, wherein the extended sequence comprises a variant region and a 3′ homologous sequence capable of hybridization to the downstream cleaved strand of the target polynucleotide.

A wide variety of reverse transcriptases (RT) may be used in alternative embodiments of the present invention, including prokaryotic and eukaryotic RT, provided that the RT functions within the host to generate a donor polynucleotide sequence from the RNA template. If desired, the nucleotide sequence of a native RT may be modified, for example using known codon optimization techniques, so that expression within the desired host is optimized. A reverse transcriptase (RT) is an enzyme used to generate complementary DNA (cDNA) from an RNA template, a process termed reverse transcription. Reverse transcriptases are used by retroviruses to replicate their genomes, by retrotransposon mobile genetic elements to proliferate within the host genome, by eukaryotic cells to extend the telomeres at the ends of their linear chromosomes, and by some non-retroviruses such as the hepatitis B virus, a member of the Hepadnaviridae, which are dsDNA-RT viruses. Retroviral RT has three sequential biochemical activities: RNA-dependent DNA polymerase activity, ribonuclease H, and DNA-dependent DNA polymerase activity. Collectively, these activities enable the enzyme to convert single-stranded RNA into double-stranded cDNA. In one embodiment, the RT domain of a reverse transcriptase is used in the present invention. The domain may include only the RNA-dependent DNA polymerase activity. In some examples, the RT domain is non-mutagenic, i.e., does not cause mutation in the donor polynucleotide (e.g., during the reverse transcriptase process). In some cases, in some examples, the RT domain may be non-retron RT, e.g., a viral RT or a human endogenous RT. In some examples, the RT domain may be retron RT or DGRs RT. In some examples, the RT may be less mutagenic than a counterpart wildtype RT. In one embodiment, the RT herein is not mutagenic.

The reverse transcriptase may be fused to the C-terminus of a TnpB. Alternatively, or additionally, the reverse transcriptase may be fused to the N-terminus of a TnpB. The fusion may be via a linker and/or an adaptor protein. In some examples, the reverse transcriptase may be an M-MLV reverse transcriptase or variant thereof. The M-MLV reverse transcriptase variant may comprise one or more mutations. For the examples, the M-MLV reverse transcriptase may comprise D200N, L603W, and T330P. In another example, the M-MLV reverse transcriptase may comprise D200N, L603W, T330P, T306K, and W313F. In a particular example, the fusion of TnpB polypeptide and reverse transcriptase is TnpB polypeptide with mutation fused with M-MLV reverse transcriptase (D200N+L603W+T330P+T306K+W313F).

The small sizes of the TnpB polypeptide herein may allow easier packaging and delivery of the prime editing system, e.g., with a viral vector, e.g., AAV or lentiviral vector. See, e.g., Lino et al. Drug Deliv. 2018; 25(1): 1234-1257; doi: 10.1080/10717544.2018.1474964, incorporated herein by reference, specifically, see table 1, incorporated herein by reference for CRISPR delivery approaches.

A single-strand break (a nick) may be generated on the target DNA by the TnpB polypeptide at the target site to expose a 3′-hydroxyl group, thus priming the reverse transcription of an edit-encoding extension on the nucleic acid component molecule directly into the target site. These steps may result in a branched intermediate with two redundant single-stranded DNA flaps: a 5′ flap that contains the unedited DNA sequence, and a 3′ flap that contains the edited sequence copied from the nucleic acid component. The 5′ flaps may be removed by a structure-specific endonuclease, e.g., FEN122, which excises 5′ flaps generated during lagging-strand DNA synthesis and long-patch base excision repair. The non-edited DNA strand may be nicked to induce bias DNA repair to preferentially replace the non-edited strand. Examples of prime editing systems and methods include those described in Anzalone A V et al., Search-and-replace genome editing without double-strand breaks or donor DNA, Nature. 2019 Oct. 21. doi: 10.1038/s41586-019-1711-4, which is incorporated by reference herein in its entirety.

The TnpB (e.g., the nickase form) may be used to prime-edit a single nucleotide on a target DNA. Alternatively or additionally, the TnpB polypeptide may be used to prime-edit at least 2, at least 3, at least 4, at least 5, at least 6, at least 7, at least 8, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 200, at least 300, at least 400, at least 500, at least 600, at least 700, at least 800, at least 900, or at least 1000 nucleotides on a target DNA.

In yet another embodiment, PRIME editing is used first to create a longer 3′ region (e.g., 20 nucleotides). Examples of prime editing systems and methods include those described in Anzalone A V et al., Search-and-replace genome editing without double-strand breaks or donor DNA, Nature. 2019 Oct. 21. doi: 10.1038/s41586-019-1711-4, which is incorporated by reference herein in its entirety. In such cases, the system comprises a TnpB protein with nickase activity, a reverse transcriptase domain, and a DNA polymerase, and a ωRNA molecule comprising a binding sequence capable of hybridizing to the target polynucleotide and an editing sequence. The generated region may be further extended on a DNA template as described herein. The latter may allow generation of a target-independent sequence, compatible with a generic donor sequence.

The TnpB protein is capable of generating a first cleavage in the target sequence and a second cleavage outside the target sequence on the target polynucleotide. In some variations, a second TnpB-mediated cleavage in vicinity to the target site may be made, which may enable more efficient invasion of the extended DNA.

In some examples, the compositions and systems of the TnpB protein herein comprise: a reverse transcriptase (RT) polypeptide connected to or otherwise capable of forming a complex with the TnpB protein; a first ωRNA molecule capable of forming a first TnpB-Reverse transcriptase complex with the TnpB protein and comprising: a ωRNA sequence capable of directing site-specific binding of the first TnpB-Reverse transcriptase complex to a first target sequence of a target polynucleotide; a first binding site region capable of binding to a cleaved or nicked strand of the target polynucleotide; and a RT template sequence encoding a first extended sequence; a second ωRNA molecule capable of forming a second TnpB-Reverse transcriptase complex with the TnpB protein and comprising: a ωRNA sequence capable of directing site specific binding of the second TnpB-Reverse transcriptase complex to a second target sequence of the target polynucleotide; a second binding site region capable of binding to a cleaved or nicked strand of the target polynucleotide; and a RT template sequence encoding a second extended sequence.

In some cases, the compositions and systems may further comprise: a donor template; a third ωRNA sequence capable of forming a TnpB-Reverse transcriptase complex-ωRNA with the TnpB protein and comprising: a ωRNA sequence capable of directing site-specific binding to a target sequence on the donor template; a third binding region capable of binding to a cleaved or nicked strand of the donor template; and a RT template encoding a third extended region complementary to the first extended region generated on the target polynucleotide; and a fourth ωRNA sequence capable of forming a TnpB-Reverse transcriptase complex with the TnpB protein and comprising: a ωRNA sequence capable of directing site-specific binding to a second target sequence on the donor template; a fourth binding region capable of binding to a cleaved or nicked strand of the donor template; and a RT template encoding a fourth extended region complementary to the second extended region generated on the target polynucleotide.

Advantageously, the more open configuration of the TnpB complex relative to a CRISPR-Cas enzyme can allow for improved accessibility not only for a sequence complementary to the target site, but also for the RT template sequence and the reverse transcriptase, which may increase the editing efficiency at the target site. Additionally, the more minimal size of the TnpB allows for increased packaging efficiency for delivery with the reverse transcriptase, accessibility for deliver of additional ωRNA sequences, as detailed further below, all of which may further improvise editing efficiencies.

In some cases, the compositions and systems may further comprise a site-specific recombinase, and wherein the first and second extended regions are complementary to each other and introduce a serine integrase recombination site; and a donor molecule comprising a donor sequence for insertion into the target polypeptide and the complementary recombination site to the serine integrase recombination site.

In some examples, the compositions and systems may further comprise a recombinase. The recombinase is connected to or otherwise capable of forming a complex with the TnpB protein. In one embodiment, the complex is capable of inserting a recombination site in the DNA loci of interest by extension of RT templates that encode for the recombination site on the 3′ extension of the ωRNA sequences by the reverse transcriptase. In one embodiment, a donor template comprising a compatible recombination site is provided that can recombine unidirectionally with the inserted recombination site when a recombinase specific for the recombination site is also provided. In one embodiment, the donor template is a plasmid comprising the complementary recombination site and any sequence for insertion at the DNA loci of interest. In one embodiment, the recombinase is connected to or capable of forming a complex with the TnpB enzyme, such that all of the enzymatic proteins are brought into contact at the loci of interest. In one embodiment, the recombinase is codon optimized for eukaryotic cells (described further herein). In one embodiment, the recombinase includes a NLS (described further herein). In one embodiment, the recombinase is provided as a separate protein. The separate recombinase may form a dimer and bind to the donor template recombination site. The recombinase may be targeted to the loci of interest as a result of the insertion of the compatible recombination site that is also recognized by the recombinase. Thus, the recombinase may recognize the recombination site inserted at the DNA loci of interest and the recombination site on the donor and be targeted to the DNA loci of interest without any additional modifications to the recombinase.

In one embodiment, a second TnpB complex connected to a recombinase is targeted to the DNA loci of interest. In one embodiment, the second TnpB complex comprises a dead TnpB protein (dTnpB, described further herein), such that the recombinase is targeted to the DNA loci of interest, but the target sequence is not further cleaved. In one embodiment, the dTnpB targets a sequence generated only after the insertion of the recombination site. In one embodiment, the recombinase recognizes and binds to the donor template recombination site and the inserted recombination site. In one embodiment, the recombinase forms a dimer with a recombinase provided as a separate protein.

As used herein, the term “recombinase” refers to an enzyme that catalyzes recombination between two or more recombination sites (e.g., an acceptor and donor site). Recombinases useful in the present invention catalyze recombination at specific recombination sites which are specific polynucleotide sequences that are recognized by a particular recombinase. “Uni-directional recombinases” or “integrases” refer to recombinase enzymes whose recognition sites are destroyed after the recombination has taken place. The term “integrase” refers to a type of recombinase. In other words, the sequence recognized by the recombinase is changed into one that is not recognized by the recombinase upon recombination. As a result, once a sequence is subjected to recombination by the uni-directional recombinase, the continued presence of the recombinase cannot reverse the previous recombination event.

“Recombination sites” are specific polynucleotide sequences that are recognized by the recombinase enzymes described herein. Typically, two different sites are involved (in regard to recombination termed “complementary sites”), one present in the target nucleic acid (e.g., a chromosome or episome of a eukaryote) and another on the nucleic acid that is to be integrated at the target recombination site. The terms “attB” and “attP,” which refer to attachment (or recombination) sites originally from a bacterial target (attachment site of bacteria) and a phage donor (attachment site of phage), respectively, are used herein although recombination sites for particular enzymes may have different names. The two attachment sites can share as little sequence identity as a few base pairs. The recombination sites typically include left and right arms separated by a core or spacer region. Thus, an attB recombination site consists of BOB′, where B and B′ are the left and right arms, respectively, and O is the core region. Similarly, attP is POP′, where P and P′ are the arms and O is again the core region. Upon recombination between the attB and attP sites, and concomitant integration of a nucleic acid at the target, the recombination sites that flank the integrated DNA are referred to as “attL” and “aatR.” The attL and attR sites, using the terminology above, thus consist of BOP′ and POB′, respectively. In some representations herein, the “O” is omitted and attB and attP, for example, are designated as BB′ and PP′, respectively.

### TnpB-Associated Transposase Systems

The systems and compositions herein may comprise a TnpB, one or more nucleic acid components, and one or more components of a transposase. In one example embodiment, the TnpB mediates RNA-guided TnpA-catalyzed transposition. In one-example embodiment, TnpB mediate RNA-guided Tn7-catalyzed transposition.

In an example embodiment, the transposases may comprise TnpA. The transposase may be a Y1 transposase of the IS200/IS605 family, encoded by the insertion sequence (IS) IS608 from Helicobacter pylori, e.g., TnpAIS608, from Deinococcus radiodurans, e.g., ISDra2, from Halanaerobium hydrogeniformans or from Sulfolobus solfataricus. Examples of the transposases include those described in Barabas, O., Ronning, D. R., Guynet, C., Hickman, A. B., TonHoang, B., Chandler, M. and Dyda, F. (2008) Mechanism of IS200/IS605 family DNA transposases: activation and transposon-directed target site selection. Cell, 132, 208-220; in Sadler et al., Genes 2020, 11, 484, doi: 10.3390/genes11050484, and in He et al., (2013) NAR, 41:5, 3302-3313. In certain example embodiments, the transposase is a single stranded DNA transposase. In certain example embodiments, the single stranded DNA transposase is TnpA or a functional fragment thereof. In an aspect, the TnpA motif used for homing to an insertion site is at least 50%, 75% or 100% complementary to the TAM of the TnpB, such that TnpA catalyzed transposition may occur at or near the TAM portion of the sequence.

In some examples, the one or more transposases or transposase sub-units are, or are derived from, Tn7 transposases. In a particular embodiment, the Tn7 or TN7-like transposase may be a Tn5053 transposase. For example, the Tn5053 transposases include those described in Minakhina S et al., Tn5053 family transposons are res site hunters sensing plasmidal res sites occupied by cognate resolvases. Mol Microbiol. 1999 September; 33(5): 1059-68; and FIG. 4 and related texts in Partridge S R et al., Mobile Genetic Elements Associated with Antimicrobial Resistance, Clin Microbiol Rev. 2018 Aug. 1; 31(4), both of which are incorporated by reference herein in their entirety. In some cases, the one or more Tn5053 transposases may comprise one or more of TniA, TniB, and TniQ. TniA is also known as TnsB. TniB is also known as TnsC. TniQ is also known as TnsD. Accordingly, in one embodiment these Tn5053 transposase subunits may be referred to as TnsB, TnsC, and TnsD, respectively. In certain cases, the one or more transposases may comprise TnsB, TnsC, and TnsD.

In one embodiment, the transposases may be one or more Vibrio cholerae Tn6677 transposases. In one example, the transposon may include a terminal operon comprising the tnsA, tnsB, and tnsC genes. The transposon may further comprise a tniQ gene. In one embodiment, the TnsE may be absent in the transposon.

In certain examples, the transposase includes one or more of Mu-transposase, TniQ, TniB, or functional domains thereof. In certain examples, the transposase includes one or more of TniQ, a TniB, a TnpB, or functional domains thereof. In certain examples, the transposase includes one or more of a rve integrase, TniQ, TniB, or functional domains thereof.

In one embodiment the system, more particularly the transposase does not include an rve integrase. In one embodiment the system, more particularly the transposase does not include one or more of Mu-transposase, TniQ, a TniB, a TnpB, a IstB domain or functional domains thereof.

In certain examples, the transposase includes one or more of Mu-transposase, TniQ, TniB, or functional domains thereof. In certain examples, the transposase includes one or more of TniQ, a TniB, a TnpB, or functional domains thereof. In certain examples, the transposase includes one or more of a rve integrase, TniQ, TniB, TnpB domain, or functional domains thereof.

A right end sequence element or a left end sequence element are made in reference to an example Tn7 transposon. The general structure of the left end (LE) and right end (RE) sequence elements of canonical Tn7 is established. Tn7 ends comprise a series of 22-bp TnsB-binding sites. Flanking the most distal TnsB-binding sites is an 8-bp terminal sequence ending with 5′-TGT-3′/3′-ACA-5′. The right end of Tn7 contains four overlapping TnsB-binding sites in the ˜90-bp right end element. The left end contains three TnsB-binding sites dispersed in the ˜150-bp left end of the element. The number and distribution of TnsB-binding sites can vary among Tn7-like elements. End sequences of Tn7-related elements can be determined by identifying the directly repeated 5-bp target site duplication, the terminal 8-bp sequence, and 22-bp TnsB-binding sites (Peters J E et al., 2017). Example Tn7 elements, including right end sequence element and left end sequence element include those described in Parks A R, Plasmid, 2009 January; 61(1): 1-14.

### TnpB Recombinase/Integrase Systems

The systems and compositions herein may comprise a TnpB system, and one or more components of a recombinase o rintegrase. In an aspect, the TnpB is naturally catalytically inactive and utilized with one or more nucleic acid components to provide site-specific targetings, and the one or more components of the recombinase to introduce a modification. In an aspect, the TnpB polypeptide may be catalytically inactivated via mutation of one or more residues of a catalytic domain (e.g., RuvC) or via truncation, and utilized with one or more nucleic acid components to provide site-specific targeting, and the one or more components of the recombinase introduce a modification. In one embodiment, a naturally inactive TnpB is provided with a recombinase, e.g., an integrase, and optionally a reverse transcriptase. The systems and compositions herein may comprise a TnpB polypeptide, one or more nucleic acid components, and one or more components of an integrase. In an aspect, the TnpB polypeptide is a nickase, and utilized with one or more nucleic acid components to provide site-specific targeting, with the one or more components of the integrase introduce a modification. The systems and compositions may be used to insert a donor polynucleotide to a target polynucleotide. The systems and compositions may further comprise a donor polynucleotide.

In preferred embodiments, the recombinase mediates unidirectional site-specific recombination. In one embodiment, the recombinase is a serine recombinase (SR) also referred to as a serine integrase, encoded, for example, by IS607 family, Tn4451, and bacteriophage phiC31. See, generally, Smith M C, Thorpe H M: Diversity in the serine recombinases. Mol Microbiol. 2002, 44:299-307. 10.1046/j. 1365-2958.2002.02891.x; Li et al., (2018) J. Mol. Biol. 430:21, 4401-4418.

In an embodiment, the recombinase is a tyrosine recombinase (YR) encoded by IS91, Helitron, IS200/IS605, Crypton or DIRS-retrotransposon families. See, generally, Goodwin T J, Butler M I, Poulter T: Cryptons: a group of tyrosine-recombinase-encoding DNA transposons from pathogenic fungi. Microbiology. 2003, 149:3099-3109. Doi: 10.1099/mic.0.26529-0; Cappello J, Handelsman K, Lodish H F: Sequence of Dictyostelium DIRS-1: an apparent retrotransposon with inverted terminal repeats and an internal circle junction sequence. Cell. 1985, 43:105-115. 10.1016/0092-8674(85) 90016-9.

In an aspect, the recombinase provides site-specific integration of a template that can be provided with the composition, e.g., a donor oligonucleotide. Without being bound by theory, the recombinase allows for integration independent of payload size and can coordinate strand exchange and re-ligation across multiple cell types, allowing integration of long stretches of polynucleotides. In an exemplary embodiment, the serine recombinase is PhiC31 and the target is DNA. In an aspect, the phiC31 allows for integration of a target site comprising an attP or pseudoattP recognition site. See, e.g., systembio.com/wp-content/uploads/phiC31_productsheet-1.pdf. In an embodiment utilizing phiC231, a donor oligonucleotide would be provided with an attB at sequence that facilitates attachment at the attP site of the target genome. Similar approaches of designing donor oligonucleotides with sequences complementary to attachment sites for a recombinase can be designed for use with the present invention. See, e.g., Li et al., (2018) J. Mol. Biol. 430:21, 4401-4418.

In preferred embodiments, the integrase mediates gene integration at diverse loci by directing insertion with an TnpB nickase fused to both a reverse transcriptase and an integrase. In one embodiment, the integrase is a serine integrase, encoded, for example, BxbINT. See, generally, Ioannidi et al., “Drag-and-drop genome insertion without DNA cleavage with CRISPR-directed integrases”; doi: 10.1101/2021.11.01.466786m incorporated herein by reference in its entirety. In Ioannidi, Gootenberg, Abudayyeh, and colleagues show integration using a CRISPR-Cas9 nickase fused to a reverse transcriptase and serine integrase termed Programmable Addition via Site-specific Targeting Elements (PASTE) with delivery via a single dose of plasmids with functionality in non-dividing and primary cells, utilizing a guide RNA comprising an AttB landing site, termed attachment site-containing guide RNA were used to insert sequences, including diverse cargo sequences that can be inserted across different loci, varying in size up to about 36 kb. Additional uses of the PASTE system included gene tagging, gene replacement, gene delivery, and protein production and secretion, approaches that are contemplated for use with the TnpB nickase and integrase approach. In an aspect, the omega RNA may comprise an AttB landing site. In an aspect, the recombinase provides site-specific integration of a template that can be provided with the composition, e.g., a donor oligonucleotide.

Additional large serine integrases can be used with the TnpB polypeptide, for example, as identified and described in Durrant et al., Large-scale discovery of recombinases for integrating DNA into the human genome, doi: 10.1101/2021.11.05.467528, incorporated herein by reference. Other integrases include BceINT, SscINT, SacINT. See, Ioannidi, 2021 at and FIG. 6d, and FIG. 10a.

Without being bound by theory, the recombinase allows for integration independent of payload size and can coordinate strand exchange and re-ligation across multiple cell types, allowing integration of long stretches of polynucleotides. In an exemplary embodiment, the integrase is BxbINT and the target is DNA. In an aspect, the BxbINT allows for integration of a target site comprising an attP or pseudoattP recognition site. In an embodiment utilizing BxbINT, a donor oligonucleotide would be provided with an att at sequence that facilitates attachment at the attP site of the target genome. Similar approaches of designing donor oligonucleotides with sequences complementary to attachment sites for an integrase can be designed for use with the present invention, for example a circular double-strand DNA template containing the AttP attachment site, or delivery of large cargo via an adenovirus or other viral vector, as described elsewhere herein. See, e.g., Ioannidi et al., 2021 at FIGS. 1a, 1b and 5b.

### TnpB Topoisomerase Systems

The one or more functional domains may be one or more topoisomerase domains. Topoisomerases are a class of enzymes that modify the topological state of DNA via the breakage and rejoining of nucleic acid strands. In some cases, a topoisomerase may be a DNA topoisomerase, which is an enzyme that controls and alters the topologic states of DNA during transcription, and catalyzes the transient breaking and rejoining of a single strand of DNA which allows the strands to pass through one another, thus altering the topology of DNA.

In one embodiment, the topoisomerase domain is capable of ligating the donor polynucleotide with the target polynucleotide. The ligation may be achieved by sticky end or blunt end ligation. In an example, a donor polynucleotide may comprise a overhang comprising a sequence complementary to a region of the target polynucleotide. Examples of ligating the donor polynucleotide with the target polynucleotide include those of TOPO cloning, e.g., those described in “The Technology Behind TOPO Cloning,” at www.thermofisher.com/us/en/home/life-science/cloning/topo/topo-resources/the-technology-behind-topo-cloning.html.

In one embodiment, the topoisomerase domain may be associated with a donor polynucleotide. For example, the topoisomerase domain is covalently linked to a donor polynucleotide. In one embodiment, a topoisomerase domain may be provided together with, e.g., associated (e.g., fused) with a TnpB or a variant thereof.

Alternatively or additionally, the topoisomerase domain may be on a molecule different from TnpB polypeptide. In some cases, the topoisomerase domain may be associated with a donor polynucleotide. For example, the topoisomerase domain may be pre-loaded covalently with a donor DNA molecule. Such deign may allow for efficient ligation of only a specific cargo. The topoisomerase domain may ligate the donor polynucleotide (e.g., a DNA molecule) to a target site on a target polynucleotide (e.g., a free double-stranded DNA end). In one embodiment, the donor polynucleotide may have an overhang that comprises a sequence complementary to a region of the target polynucleotide. For example, the overhang may invade into the target polynucleotide at a cut site generated by the tnpB polypeptide.

Examples of topoisomerases include type I, including type IA and type IB topoisomerases, which cleave a single strand of a double-stranded nucleic acid molecule, and type II topoisomerases (e.g., gyrases), which cleave both strands of a double-stranded nucleic acid molecule.

Type IA and IB topoisomerases cleave one strand of a double-stranded nucleic acid molecule. In some examples, the cleavage of a double-stranded nucleic acid molecule by type IA topoisomerases generates a 5′ phosphate and a 3′ hydroxyl at the cleavage site, with the type IA topoisomerase covalently binding to the 5′ terminus of a cleaved strand. Cleavage of a double-stranded nucleic acid molecule by type IB topoisomerases may generate a 3′ phosphate and a 5′ hydroxyl at the cleavage site, with the type IB topoisomerase covalently binding to the 3′ terminus of a cleaved strand.

Examples of Type IA topoisomerases include E. coli topoisomerase I, E. coli topoisomerase III, eukaryotic topoisomerase II, archeal reverse gyrase, yeast topoisomerase III, Drosophila topoisomerase III, human topoisomerase III, Streptococcus pneumoniae topoisomerase III, and the like, including other type IA topoisomerases. A DNA-protein adduct is formed with the enzyme covalently binding to the 5′-thymidine residue, with cleavage occurring between the two thymidine residues.

Examples of Type IB topoisomerases include the nuclear type I topoisomerases present in all eukaryotic cells and those encoded by Vaccinia and other cellular poxviruses. The eukaryotic type IB topoisomerases are exemplified by those expressed in yeast, Drosophila and mammalian cells, including human cells. Viral type IB topoisomerases are exemplified by those produced by the vertebrate poxviruses (Vaccinia, Shope fibroma virus, ORF virus, fowlpox virus, and molluscum contagiosum virus), and the insect poxvirus (Amsacta moorei entomopoxvirus).

Examples of Type II topoisomerases include, bacterial gyrase, bacterial DNA topoisomerase IV, eukaryotic DNA topoisomerase II, and T-even phage encoded DNA topoisomerases. Type II topoisomerases may have both cleaving and ligating activities. Substrate double-stranded nucleic acid molecules of type II topoisomerase can be prepared such that the type II topoisomerase can form a covalent linkage to one strand at a cleavage site. For example, calf thymus type II topoisomerase can cleave a substrate ds nucleic acid molecule containing a 5′ recessed topoisomerase recognition site positioned three nucleotides from the 5′ end, resulting in dissociation of the three nucleic acid molecule 5′ to the cleavage site and covalent binding of the topoisomerase to the 5′ terminus of the ds nucleic acid molecule. Furthermore, upon contacting such a type II topoisomerase-charged ds nucleic acid molecule with a second nucleic acid molecule containing a 3′ hydroxyl group, the type II topoisomerase can ligate the sequences together, and then is released from the recombinant nucleic acid molecule.

Structural analysis of topoisomerases indicates that the members of each particular topoisomerase families, including type IA, type IB and type II topoisomerases, share common structural features with other members of the family. In addition, sequence analysis of various type IB topoisomerases indicates that the structures are highly conserved, particularly in the catalytic domain. For example, a domain comprising amino acids 81 to 314 of the 314 amino acid Vaccinia topoisomerase shares substantial homology with other type IB topoisomerases, and the isolated domain has essentially the same activity as the full length topoisomerase, although the isolated domain has a slower turnover rate and lower binding affinity to the recognition site. In addition, a mutant Vaccinia topoisomerase, which is mutated in the amino terminal domain (e.g., at amino acid residues 70 and 72) may display identical properties as the full length topoisomerase. Mutation analysis of Vaccinia type IB topoisomerase reveals a large number of amino acid residues that can be mutated without affecting the activity of the topoisomerase, and has identified several amino acids that are required for activity. In view of the high homology shared among the Vaccinia topoisomerase catalytic domain and the other type IB topoisomerases, and the detailed mutation analysis of Vaccinia topoisomerase, it will be recognized that isolated catalytic domains of the type IB topoisomerases and type IB topoisomerases having various amino acid mutations can be used in the methods of the invention and thus are considered to be topoisomerases for purposes of the present invention.

The various topoisomerases exhibit a range of sequence specificity. For example, type II topoisomerases can bind to a variety of sequences, but cleave at a highly specific recognition site. The type IB topoisomerases may include site specific topoisomerases, which bind to and cleave a specific nucleotide sequence (“topoisomerase recognition site”). Upon cleavage of a double-stranded nucleic acid molecule by a topoisomerase, for example, a type IB topoisomerase, the energy of the phosphodiester bond is conserved via the formation of a phosphotyrosyl linkage between a specific tyrosine residue in the topoisomerase and the 3′ nucleotide of the topoisomerase recognition site. Where the topoisomerase cleavage site is near the 3′ terminus of the nucleic acid molecule, the downstream sequence (3′ to the cleavage site) can dissociate, leaving a nucleic acid molecule having the topoisomerase covalently bound to the newly generated 3′ end.

The covalently bound topoisomerase also can catalyze the reverse reaction, for example, covalent linkage of the 3′ nucleotide of the recognition sequence, to which a type IB topoisomerase is linked through the phosphotyrosyl bond, and a nucleic acid molecule containing a free 5′ hydroxyl group. As such, methods have been developed for using a type IB topoisomerase to produce recombinant nucleic acid molecules. Nucleic acid molecules such as those comprising a cDNA library, or restriction fragments, or sheared genomic DNA sequences that are to be cloned into such a vector are treated, for example, with a phosphatase to produce 5′ hydroxyl termini, then are added to the linearized vector under conditions that allow the topoisomerase to ligate the nucleic acid molecules at the 5′ terminus containing the hydroxyl group and the 3′ terminus containing the covalently bound topoisomerase.

Examples of vaccinia viruses encode a 314 amino acid type I topoisomerase enzyme capable of site-specific single-strand nicking of double stranded DNA, as well as 5′ hydroxyl driven re-ligation. Site-specific type I topoisomerases include, but are not limited to, viral topoisomerases such as pox virus topoisomerase. Examples of pox virus topoisomerases include Shope fibroma virus and ORF virus. Other site-specific topoisomerases are well known to those skilled in the art and can be used to practice this invention.

Examples of vaccinia topoisomerase binds to duplex DNA and cleaves the phosphodiester backbone of one strand while exhibiting a high level of sequence specificity. Cleavage may occur at a consensus pentapyrimidine element 5′-(C/T) CCTTI, or related sequences in the scissile strand. In one embodiment the scissile bond is situated in the range of 2 to 12 bp from the 3′ end of the duplex DNA. In another embodiment cleavable complex formation by Vaccinia topoisomerase requires six duplex nucleotides upstream and two nucleotides downstream of the cleavage site.

In some examples, the topoisomerase is DNA topoisomerase I, e.g., a Vaccinia virus topoisomerase I. The topoisomerase may be pre-loaded with a donor polynucleotide. The Vaccinia virus topoisomerase may need a target comprising a 5′ —OH group.

### TnpB Guided Excision-Transposition Systems

Embodiments disclosed herein provide an engineered or non-natural guided excision-transposition system. The engineered or non-natural guided excision-transposition system may comprise one or more components of a ωRNA-TnpB system, e.g., an ωRNA scaffold and spacer and/or TnpB polypeptide, and one or more components of a Class II transposon. The components of the ωRNA-TnpB system can direct the Class II transposon component(s) to retrotransposon to a target nucleic acid sequence and direct its transposition into a recipient polynucleotide.

For example, the engineered or non-natural guided excision-transposition systems that can include (a) a first TnpB protein; (b) a first Class II transposon polypeptide coupled to or otherwise capable of complexing with the first TnpB protein; (c) a first guide molecule capable of forming a first ωRNA-TnpB complex with the first TnpB protein and directing site-specific binding to a first target sequence of a first target polynucleotide; (d) a second TnpB protein; (e) a second Class II transposon polypeptide coupled to or otherwise capable of complexing with the second TnpB protein; (f) a second guide molecule capable of forming a second ωRNA-TnpB complex with the first TnpB protein and directing site-specific binding to a second target sequence of the first target polynucleotide; and (g) a Class II transposon polynucleotide comprising the first target polynucleotide and is capable of forming a complex with the first and second TnpB protein, the first and second guide molecules, and the first and second Class II transposon polypeptides.

In some embodiments, the engineered or non-natural guided excision-transposition system can include (h) a third guide molecule capable of complexing with the first TnpB protein and directing site-specific binding to a first target sequence of a second target polynucleotide, wherein the third guide molecule is optionally coupled to the first TnpB protein; (i) optionally, a first guide molecule polynucleotide that encodes the third guide molecule; (j) a fourth guide molecule capable of complexing with the second TnpB protein and directing site-specific binding to a second target sequence of the second target polynucleotide, wherein the fourth guide molecule is optionally coupled to the second TnpB protein; and (k) optionally, a second guide molecule polynucleotide that encodes the fourth guide molecule.

In some embodiments, the first and the second Class II transposon polypeptides are capable of excising the first target polynucleotide from the Class II transposon polynucleotide. In some embodiments, the first and the second Class II transposon polypeptides are capable of transposing the first target polynucleotide in the second target polynucleotide. In some embodiments, the first target polynucleotide does not include one or more Class II transposon long terminal repeats.

The engineered or non-natural guided excision-transposition systems described herein can be based on a Class II transposon or Class II transposon system. The engineered or non-natural guided excision-transposition system may include a first target polynucleotide, also referred to as a donor polynucleotide or transposon and a second target polynucleotide, which is also referred to herein as a recipient polynucleotide. As used herein, “transposon” (also referred to as transposable element) refers to a polynucleotide sequence that is capable of moving form location in a genome to another. There are several classes of transposons. Transposons include retrotransposons (Class I transposons) and DNA transposons (Class II transposons). In some cases, retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide.

Any suitable transposon system can be used. Suitable transposon and systems thereof can include, but are not limited, to Sleeping Beauty transposon system (Tc1/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tcl/mariner superfamily) (see e.g., Miskey et al. 2003 Nucleic Acid Res. 31(23): 6873-6881) and variants thereof.

In some embodiments, the first and/or second Class II transposon polypeptide is a DD [E/D] transposon or transposon polypeptide. In some embodiments, the first and/or the second Class II transposon polynucleotide is a Tcl/mariner, PiggyBac, Frog Prince, Tn3, Tn5, hAT, CACTA, P, Mutator, PIF/Harbinger, Transib, or a Merlin/IS1016 transposon polynucleotide. In some embodiments, the first and/or second Class II transposon polypeptide is a Tcl/mariner, PiggyBac, Frog Prince, Tn3, Tn5, hAT, CACTA, P, Mutator, PIF/Harbinger, Transib, or a Merlin/IS1016 transposon polypeptide.

Suitable Class II transposon systems and components that can be utilized can also be and are not limited to those described in e.g., and without limitation, Han et al., 2013. BMC Genomics. 14:71, doi: 10.1186/1471-2164-14-71, Lopez and Garcia-Perez. 2010. Curr. Genomics. 11(2): 115-128; Wessler. 2006. PNAS. 103(47): 176000-17601; Gao et al., 2017. Marine Genomics. 34:67-77; Bradic et al. 2014. Mobile DNA. 5(12) doi: 10.1186/1759-8753-5-12; Li et al., 2013. PNAS. 110(25) E2279-E2287; Kebriaei et al. 2017. Trends in Genetics. 33(11): 852-870); Miskey et al. 2003. Nucleic Acid res. 31(23): 6873-6881; Nicolas et al. 2015. Microbiol Spectr. 3(4) doi: 10.1128/microbiolspec.MDNA3-0060-2014); W. S. Reznikoff. 1993. Annu Rev. Microbiol. 47:945-963; Rubin et al. 2001. Genetics. 158(3): 949-957; Wicker et al. 2003. Plant Physiol. 132(1): 52-63; Majumdar and Rio. 2015. Microbiol. Spectr. 3(2) doi: 10.1128/microbiolspec.MDNA3-0004-2014; D. Lisch. 2002. Trends in Plant Sci. 7(11): 498-504; Sinzelle et al. 2007. PNAS. 105(12): 4715-4720; Han et al. 2014; Genome Biol. Evol. 6(7): 1748-1757; Grzebelus et al. 2006; Mol. Genet. Genomics. 275(5): 450-459; Zhang et al. 2004. Genetics. 166(2): 971-986; Chen and Li. 2008. Gene. 408(1-2): 51-63; and C. Feschotte. 2004. Mol. Biol. Evol. 21(9): 1769-1780.

### TnpB Retrotransposon Systems

The systems and compositions herein may comprise a TnpB, one or more nucleic acid components, and one or more components of a retrotransposon, e.g., a non-LTR retrotransposon. The one or more components of a retrotransposon include a retrotransposon protein and retrotransposon RNA. The systems and compositions may be used to insert a donor polynucleotide to a target polynucleotide. The systems and compositions may further comprise a donor polynucleotide.

In some examples, the present disclosure provides an engineered, non-naturally occurring composition comprising: a TnpB polypeptide, a non-LTR retrotransposon protein associated with or otherwise capable of forming a complex with the TnpB polypeptide; a single nucleic acid component capable of forming a complex with the TnpB polypeptide and directing site-specific binding to a target sequence of a target polynucleotide. The composition may further comprise a donor construct comprising a donor polynucleotide for insertion to the target polynucleotide and located between two binding elements capable of forming a complex with the non-LTR retrotransposon protein. In some cases, the TnpB polypeptide is engineered to have nickase activity.

In some examples, the TnpB polypeptide is fused to the N-terminus of the non-LTR retrotransposon protein. In some examples, the TnpB polypeptide is fused to the C-terminus of the non-LTR retrotransposon protein.

The nucleic acid component molecule s may direct the fusion protein to a target sequence 5′ of the targeted insertion site, and wherein the TnpB polypeptide generates a double-strand break at the targeted insertion site. The nucleic acid component molecule s may direct the fusion protein to a target sequence 3′ of the targeted insertion site, and wherein the TnpB polypeptide generates a double-strand break at the targeted insertion site.

The donor polynucleotide may further comprise a polymerase processing element to facilitate 3′ end processing of the donor polynucleotide sequence. The polymerase may be a DNA polymerase, e.g., DNA polymerase I. In some examples, the polymerase may be an RNA polymerase.

In some examples, the donor polynucleotide further comprises a homology region to the target sequence on the 5′ end of the donor construct, the 3′ end of the donor construct, or both. In some examples, the homology region is from 1 to 50, from 5 to 30, from 8 to 25, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base pairs in length.

Native or wild-type non-LTR retrotransposons encode the protein machinery necessary for their self-mobilization. The non-LTR retrotransposon element comprises a DNA element integrated into a host genome. This DNA element may encode one or two open reading frames (ORFs). For example, the R2 element of Bombyx mori encodes a single ORF containing reverse transcriptase (RT) activity and a restriction enzyme-like (REL) domain. L1 elements encode two ORFs, ORF1 and ORF2. ORF1 contains a leucine zipper domain involved in protein-protein interactions and a C-terminal nucleic acid binding domain. ORF2 has a N-terminal apurinic/apyrimidinic endonuclease (APE), a central RT domain, and a C-terminal cysteine histidine rich domain. An example replicative cycle of a non-LTR retrotransposon may comprise transcription of the full-length retrotransposon element to generate an mRNA active element (retrotransposon RNA). The active element mRNA is translated to generate the encoded retrotransposon proteins or polypeptides. A ribonucleoprotein complex comprising the active element and retrotransposon protein or polypeptide is formed and this RNP facilitates integration of the active element into the genome. The RNA-transposase complex nicks the genome. The 3′ end of the nicked DNA serves as a primer to allow the reverse transcription of the transposon RNA into cDNA. Fourth, the transposase proteins integrate the cDNA into the genome.

Elements of these systems may be engineered to work within the context of the invention. For example, a non-LTR retrotransposon polypeptide may be fused to a site-specific nuclease. The binding elements that allow a non-LTR retrotransposon polypeptide to bind to the native retrotransposon DNA element, may be engineered into a donor construct to facilitate entry of a donor polynucleotide sequence into a target polypeptide.

In the present invention, the protein component of the non-LTR retrotransposon may be connected to or otherwise engineered to form a complex with a site-specific nuclease, e.g., TnpB polypeptide. The retrotransposon RNA may be engineered to encode a donor polynucleotide sequence. Thus, in certain example embodiments, the TnpB polypeptide, via formation of a TnpB polypeptide complex with a nucleic acid component molecule sequence, directs the retrotransposon complex (e.g., the retrotransposon polypeptide(s) and retrotransposon RNA to a target sequence in a target polynucleotide, where the retrotransposon RNP complex facilitates integration of the donor polynucleotide sequence into the target polynucleotide. Accordingly, the one or more non-LTR retrotransposon components may comprise retrotransposon polypeptides, or function domains thereof, that facilitate binding of the retrotransposon RNA, reverse transcription of the retrotransposon RNA into cDNA, and/or integration of the donor polynucleotide into the target polynucleotide, as well as retrotransposon RNA elements modified to encode the donor polynucleotide sequence.

Examples non-LTR retrotransposons include CRE, R2, R4, L1, RTE, Tad, R1, LOA, I, Jockey, CR1. In one example, the non-LTR retrotransposon is R2. In another example, the non-LTR retrotransposon is L1. Examples of non-LTR retrotransposons may include those described in Christensen S M et al., RNA from the 5′ end of the R2 retrotransposon controls R2 protein binding to and cleavage of its DNA target site, Proc Natl Acad Sci USA. 2006 Nov. 21; 103(47): 17602-7; Eickbush T H et al, Integration, Regulation, and Long-Term Stability of R2 Retrotransposons, Microbiol Spectr. 2015 April; 3(2): MDNA3-0011-2014. doi: 10.1128/microbiolspec.MDNA3-0011-2014; Han J S, Non-long terminal repeat (non-LTR) retrotransposons: mechanisms, recent developments, and unanswered questions, Mob DNA. 2010 May 12; 1(1): 15. doi: 10.1186/1759-8753-1-15; Malik H S et al., The age and evolution of non-LTR retrotransposable elements, Mol Biol Evol. 1999 June; 16(6): 793-805, which are incorporated by reference herein in their entireties.

Examples of the non-LTR retrotransposon polypeptides also include R2 from Clonorchis sinensis, or Zonotrichia albicollis.

A non-LTR retrotransposon may comprise multiple retrotransposon polypeptides or polynucleotides encoding same. In one embodiment, the retrotransposon polypeptides may form a complex. For example, a non-LTR retrotransposon is a dimer, e.g., comprising two retrotransposon polypeptides forming a dimer. The dimer subunits may be connected or form a tandem fusion. A TnpB polypeptide may be associate with (e.g., connected to) one or more subunits of such complex. In some examples, the non-LTR retrotransposon is a dimer of two retrotransposon polypeptides; one of the retrotransposon polypeptides comprises nuclease or nickase activity and is connected with a TnpB polypeptide.

The retrotransposon polypeptides may comprise one or more modifications to, for example, enhance specificity or efficiency of donor polynucleotide recognition, target-primed template recognition (TPTR). The retrotransposon polypeptides may also comprise one or more truncations or excisions to remove domains or regions of wild-type protein to arrive at a minimal polypeptide that retain donor polynucleotide recognition and TPTR. In some example embodiments, the native endonuclease activity may be mutated to eliminate endonuclease activity.

In certain example embodiments, the modifications or truncations of the non-LTR retrotransposon peptide may be in a zinc finger region, a Myb region, a basic region, a reverse transcriptase domain, a cysteine-histidine rich motif, or an endonuclease domain.

A non-LTR retrotransposon may comprise polynucleotide encoding one or more retrotransposon RNA molecules. The polynucleotide may comprise one or more regulatory elements. The regulatory elements may be promoters. The regulatory elements and promoters on the polynucleotides include those described throughout this application. For example, the polynucleotide may comprise a pol2 promoter, a pol3 promoter, or a T7 promoter.

In some cases, the polynucleotide encodes a retrotransposon RNA with at least a portion of its sequence complementary to a target sequence. For example, the 3′ end of the retrotransposon RNA may be complementary to a target sequence. The RNA may be complementary to a portion of a nicked target sequence. In one embodiment, a retrotransposon RNA may comprise one or more donor polynucleotides. In certain cases, a retrotransposon RNA may encode one or more donor polynucleotides.

A retrotransposon RNA may be capable of binding to a retrotransposon polypeptide. Such retrotransposon RNA may comprise one or more elements for binding to the retrotransposon polypeptide. Examples of binding elements include hairpin structures, pseudoknots (e.g., a nucleic acid secondary structure containing at least two stem-loop structures in which half of one stem is intercalated between the two halves of another stem), stem loops, and bulges (e.g., unpaired stretches of nucleotides located within one strand of a nucleic acid duplex). In certain examples, the retrotransposon RNA comprises one or more hairpin structures. In some examples, the retrotransposon RNA comprises one or more pseudoknots. In certain examples, a retrotransposon RNA comprises a sequence encoding a donor polynucleotide and one or more binding elements for forming a complex with the retrotransposon polypeptide. The binding elements may be located on the 5′ end or the 3′ end.

In one embodiment, a retrotransposon RNA comprises a region capable of hybridizing with an overhang of a target polynucleotide at the target site. The overhang may be a stretch of single-stranded DNA. The overhang may function as a primer for reverse transcription of at least a portion of the retrotransposon RNA to a cDNA. In some cases, a region of the cDNA may be capable of hybridizing a second overhang of the target polynucleotide. The second overhang may function as a primer for the synthesis of a second strand to generate a double-stranded cDNA. The cDNA may comprise a donor polynucleotide sequence. The two overhangs may be from different strands of the target polynucleotide.

### Reverse Transcriptase Domain

The one or more functional domains may be one or more reverse transcriptase domains. In some embodiments, the systems comprise an engineered system for modifying a target polynucleotide comprising: a TnpB protein or a variant thereof (e.g., dTnpB); a reverse transcriptase (RT) domain; an RNA template comprising or encoding a donor polynucleotide to be inserted to a target sequence of the target polynucleotide; and an ωRNA molecule (i.e., a naturally single guide RNA molecule comprising a scaffold for reprogramming).

The reverse transcriptase may generate single-strand DNA based on the RNA template. The single-strand DNA may be generated by a non-retron, retron, or diversity generating retroelement (DGR). In some examples, the single-strand DNA may be generated from a self-priming RNA template. A self-priming RNA template may be used to generate a DNA without the need of a separate primer.

A reverse transcriptase domain may be a reverse transcriptase or a fragment thereof. A wide variety of reverse transcriptases (RT) may be used in alternative embodiments of the present invention, including prokaryotic and eukaryotic RT, provided that the RT functions within the host to generate a donor polynucleotide sequence from the RNA template. If desired, the nucleotide sequence of a native RT may be modified, for example using known codon optimization techniques, so that expression within the desired host is optimized. A reverse transcriptase (RT) is an enzyme used to generate complementary DNA (cDNA) from an RNA template, a process termed reverse transcription. Reverse transcriptases are used by retroviruses to replicate their genomes, by retrotransposon mobile genetic elements to proliferate within the host genome, by eukaryotic cells to extend the telomeres at the ends of their linear chromosomes, and by some non-retroviruses such as the hepatitis B virus, a member of the Hepadnaviridae, which are dsDNA-RT viruses. Retroviral RT has three sequential biochemical activities: RNA-dependent DNA polymerase activity, ribonuclease H, and DNA-dependent DNA polymerase activity. Collectively, these activities enable the enzyme to convert single-stranded RNA into double-stranded cDNA. In one embodiment, the RT domain of a reverse transcriptase is used in the present invention. The domain may include only the RNA-dependent DNA polymerase activity. In some examples, the RT domain is non-mutagenic, i.e., does not cause mutation in the donor polynucleotide (e.g., during the reverse transcriptase process). In some examples, the RT domain may be non-retron RT, e.g., a viral RT or a human endogenous RT. In some examples, the RT domain may be retron RT or DGRs RT. In some examples, the RT may be less mutagenic than a counterpart wildtype RT. In some embodiments, the RT herein is not mutagenic.

### Retrons

In one embodiment, a donor template for homologous recombination is generated by use of a self-priming RNA template for reverse transcription. A non-limiting example of a self-priming reverse transcription system is the retron system. By the term “retron” it is meant a genetic element which encodes components enabling the synthesis of branched RNA-linked single stranded DNA (msDNA) and a reverse transcriptase. Retrons which encode msDNA are known in the art, for example, but not limited to U.S. Pat. Nos. 6,017,737; 5,849,563; 5,780,269; 5,436,141; 5,405,775; 5,320,958; CA 2,075,515; all of which are herein incorporated by reference.

In one embodiment, the reverse transcriptase domain is a retron RT domain. In one embodiment, the RNA template encodes a retron RNA template that is recognized and reverse transcribed by the retron reverse transcriptase domain. Conserved across many bacterial species, retrons are highly efficient reverse transcription systems of relatively unknown function. The retron system consists of the retron RT protein, as well as the msr and msd transcripts, which function as the primer and template sequences, respectively. All components of the retron system are expressed from a single open reading frame as a single transcript including the msr-msd and encoding the retron RT protein (Lampson, et al., 2005, Retrons, msDNA, and the bacterial genome. Cytogenet Genome Res 110:491-499). The msr element ORF of a retron provides for the RNA portion of the msDNA molecule, while the msd element ORF provides for the DNA portion of the msDNA molecule. The primary transcript from the msr-msd region is thought to serve as both a template and a primer to produce the msDNA. Synthesis of msDNA is primed from an internal rG residue of the RNA transcript using its 2′ —OH group. Modification of msd, or msr may also be made to permit insertion of an RNA template encoding a donor polynucleotide within the msd without altering the functioning of or the production of msDNA. The RNA template encoding a donor polynucleotide sequence may be any length but is preferably less than about 5 kb nucleotides, or also less than about 2 kb, or also less than 500 bases, provided that an msDNA product is produced.

### TnpB Diversity Generating Retroelement Systems

In one embodiment, the one or more functional domains may be a diversity generating retroelement(s) (e.g., DGR described in US20100041033A1). In some embodiments, the DGR may insert a donor polynucleotide with its homing mechanism. For example, the DGR may be associated with a catalytically inactive TnpB protein (e.g., a dead TnpB), and integrate the single-strand DNA using a homing mechanism. In some examples, the DGR may be less mutagenic than a counterpart wild type DGR. In some examples, the DGR is not error-prone. In some embodiments, the DGR herein is not mutagenic. The non-mutagenic DGR may be a mutant of a wild type DGR. As used herein, the term “DGR” encompasses both diversity generating retroelement polynucleotides and proteins encoded by diversity generating retroelement polynucleotides. In some examples, DGR may be proteins encoded by diversity generating retroelement polynucleotides having reverse transcriptase activity. In some examples, DGR may be proteins encoded by diversity generating retroelement polynucleotides having reverse transcriptase activity and integrase activity. In some cases, the template or donor polynucleotide may be encoded by a diversity generating retroelement polynucleotide. In certain cases, the template may be a polynucleotide different from the diversity generating retroelement polynucleotide, e.g., provided as a separate construct or molecule.

In some embodiments, the DGR herein may also include a Group II intron (and any proteins and polynucleotides encoded), which are mobile ribozymes that self-splice from precursor RNAs to yield excised intron lariat RNAs, which then invade new genomic DNA sites by reverse splicing. Examples of Group II intron include those described in Lambowitz A M et al., Group II Introns: Mobile Ribozymes that Invade DNA, Cold Spring Harb Perspect Biol. 2011 August; 3(8): a003616.

In some embodiments, the diversity-generating retroelements (DGRs) are genetic elements that can produce targeted, massive variations in the genomes that carry these elements. In some embodiments, the DGR systems rely on error-prone reverse transcriptases to produce mutagenized cDNA (containing A-to-N mutations) from a template region (TR), to replace a segment called a variable region (VR) that is similar to the TR region—this process is called mutagenic retrohoming (see, e.g., Sharifi and Ye, MyDGR: a server for identification and characterization of diversity-generating retroelements. Nucleic Acids Res. 2019 Jul. 2; 47 (W1): W289-W294). DGRs may include a unique family of retroelements that generate sequence diversity of DNA. They exist widely in bacteria, archaea, phage and plasmid, and benefit their hosts by introducing variations and accelerating the evolution of target proteins (see, e.g., Yan et al., Discovery and characterization of the evolution, variation and functions of diversity-generating retroelements using thousands of genomes and metagenomes. BMC Genomics. 2019; 20:595). The first DGR was discovered in a Bordetella phage, BPP-1. Bordetella causes the respiratory infection in humans and many other mammals, controlled by the BvgAS signal transduction system. The surface of Bordetella is highly variable owing to the dynamic gene expression in the infectious cycle. The invasion of BPP-1 to Bordetella relies on the phage tail fiber protein Mtd. With the process of mutagenic reverse transcription and cDNA integration, DGR may introduce multiple nucleotide substitutions to Mtd gene and generates different receptor-binding molecules, thus making BPP-1 the ability to invade Bordetellae with diverse cell surfaces.

The systems may be used to generate an ssDNA donor using a retron- or DGR RT, which is then integrated by homologous recombination upon target cleavage or nicking using a TnpB polypeptide. In some embodiments, the systems may comprise DGRs and/or Group-II intron reverse transcriptases. The homing mechanism of DGRs or Group-II introns may be used in modifying a target polynucleotide. The DGRs or Group-II introns reverse transcriptase may be guided to a target polynucleotide by tethering to a nuclease-dead TnpB polypeptide, TALE, or ZF protein. In another embodiment, a non-retron/DGR reverse transcriptase (e.g., a viral RT) may be used for generating cDNA off of a self-priming RNA. In some embodiments, a ssDNA may be generated by an RT, but integrate it using a dead TnpB enzyme, creating an accessible R-loop instead of nicking/cleaving.

### TnpB Topoisomerase Systems

The one or more functional domains may be one or more topoisomerase domains. In some embodiments, an engineered system for modifying a target polynucleotide comprising: a TnpB protein; a topoisomerase domain; and a nucleic acid template comprising or encoding a donor polynucleotide to be inserted to a target sequence of the target polynucleotide. In some examples, two or more of: the TnpB protein; topoisomerase domain; and nucleic acid template may form a complex. In some examples, two or more of: the TnpB protein; topoisomerase domain, may be comprised in a fusion protein.

Topoisomerases are a class of enzymes that modify the topological state of DNA via the breakage and rejoining of nucleic acid strands. In some cases, a topoisomerase may be a DNA topoisomerase, which is an enzyme that controls and alters the topologic states of DNA during transcription, and catalyzes the transient breaking and rejoining of a single strand of DNA which allows the strands to pass through one another, thus altering the topology of DNA.

In some embodiments, the topoisomerase domain is capable of ligating the donor polynucleotide with the target polynucleotide. The ligation may be achieved by sticky end or blunt end ligation. In an example, the donor polynucleotide may comprise an overhang comprising a sequence complementary to a region of the target polynucleotide. Examples of ligating the donor polynucleotide with the target polynucleotide include those of TOPO cloning, e.g., those described in “The Technology Behind TOPO Cloning,” at www.thermofisher.com/us/en/home/life-science/cloning/topo/topo-resources/the-technology-behind-topo-cloning.html.

In some embodiments, the topoisomerase domain may be associated with the donor polynucleotide. For example, the topoisomerase domain is covalently linked to the donor polynucleotide.

In some embodiments, a topoisomerase domain may be provided together with, e.g., associated (e.g., fused) with a TnpB protein (e.g., a TnpB protein or a variant thereof such as a dead TnpB or a TnpB nickase). Alternatively or additionally, the topoisomerase domain may be on a molecule different from the TnpB protein. In some cases, the topoisomerase domain may be associated with a donor polynucleotide. For example, the topoisomerase domain may be pre-loaded covalently with a donor DNA molecule. Such design may allow for efficient ligation of only a specific cargo. The topoisomerase domain may ligate the donor polynucleotide (e.g., a DNA molecule) to a target site on a target polynucleotide (e.g., a free double-stranded DNA end). In some embodiments, the donor polynucleotide may have an overhang that comprises a sequence complementary to a region of the target polynucleotide. For example, the overhang may invade into the target polynucleotide at a cut site generated by the TnpB protein.

Examples of topoisomerases include type I, including type IA and type IB topoisomerases, which cleave a single strand of a double-stranded nucleic acid molecule, and type II topoisomerases (e.g., gyrases), which cleave both strands of a double-stranded nucleic acid molecule.

Type IA and IB topoisomerases cleave one strand of a double-stranded nucleic acid molecule. In some examples, the cleavage of a double-stranded nucleic acid molecule by type IA topoisomerases generates a 5′ phosphate and a 3′ hydroxyl at the cleavage site, with the type IA topoisomerase covalently binding to the 5′ terminus of a cleaved strand. Cleavage of a double-stranded nucleic acid molecule by type IB topoisomerases may generate a 3′ phosphate and a 5′ hydroxyl at the cleavage site, with the type IB topoisomerase covalently binding to the 3′ terminus of a cleaved strand.

Examples of Type IA topoisomerases include E. coli topoisomerase I, E. coli topoisomerase III, eukaryotic topoisomerase II, archeal reverse gyrase, yeast topoisomerase III, Drosophila topoisomerase III, human topoisomerase III, Streptococcus pneumoniae topoisomerase III, and the like, including other type IA topoisomerases. A DNA-protein adduct is formed with the enzyme covalently binding to the 5′-thymidine residue with cleavage occurring between the two thymidine residues.

Examples of Type IB topoisomerases include the nuclear type I topoisomerases present in all eukaryotic cells and those encoded by Vaccinia and other cellular poxviruses. The eukaryotic type IB topoisomerases are exemplified by those expressed in yeast, Drosophila and mammalian cells, including human cells. Viral type IB topoisomerases are exemplified by those produced by the vertebrate poxviruses (Vaccinia, Shope fibroma virus, ORF virus, fowlpox virus, and molluscum contagiosum virus), and the insect poxvirus (Amsacta moorei entomopoxvirus).

Examples of Type II topoisomerases include, bacterial gyrase, bacterial DNA topoisomerase IV, eukaryotic DNA topoisomerase II, and T-even phage encoded DNA topoisomerases. Type II topoisomerases may have both cleaving and ligating activities. Substrate double-stranded nucleic acid molecules of type II topoisomerase can be prepared such that the type II topoisomerase can form a covalent linkage to one strand at a cleavage site. For example, calf thymus type II topoisomerase can cleave a substrate ds nucleic acid molecule containing a 5′ recessed topoisomerase recognition site positioned three nucleotides from the 5′ end, resulting in dissociation of the three nucleic acid molecule 5′ to the cleavage site and covalent binding of the topoisomerase to the 5′ terminus of the ds nucleic acid molecule. Furthermore, upon contacting such a type II topoisomerase-charged ds nucleic acid molecule with a second nucleic acid molecule containing a 3′ hydroxyl group, the type II topoisomerase can ligate the sequences together, and then is released from the recombinant nucleic acid molecule.

In some examples, the topoisomerase is DNA topoisomerase I, e.g., a Vaccinia virus topoisomerase I. The topoisomerase may be pre-loaded with a donor polynucleotide. The Vaccinia virus topoisomerase may need a target comprising a 5′ —OH group.

### TnpB Phosphatase Systems

The systems herein may further comprise a phosphatase domain. A phosphatase is an enzyme capable of removing a phosphate group from a molecule e.g., a nucleic acid such as DNA. Examples of phosphatases include calf intestinal phosphatase, shrimp alkaline phosphatase, Antarctic phosphatase, and APEX alkaline phosphatase.

In some examples, the 5′ —OH group of in the target polynucleotide may be generated by a phosphatase. A topoisomerase compatible with a 5′ phosphate target may be used to generate stable loaded intermediates. In some cases, a TnpB polypeptide that leaves a 5′ OH after cleaving the target polynucleotide may be used. In some cases, the phosphatase domain may be associated with (e.g., fused to) the TnpB protein. The phosphatase domain may be capable of generating a —OH group at a 5′ end of the target polynucleotide. The phosphatase may be delivered separated from other components in the system, e.g., as a separate protein, on a separate vector from other components.

### TnpB Polyermase Systems

The systems herein may further comprise a polymerase domain. A polymerase refers to an enzyme that synthesizes chains of nucleic acids. The polymerase may be a DNA polymerase or an RNA polymerase.

In some embodiments, the systems comprise an engineered system for modifying a target polynucleotide comprising: a TnpB protein; a DNA polymerase domain; and a DNA template comprising a donor polynucleotide to be inserted to a target sequence of the target polynucleotide. In some examples, two or more of: the TnpB protein; DNA polymerase domain; and DNA template may form a complex. In some examples, two or more of: the TnpB protein; DNA polymerase domain; are comprised in a fusion protein. For example, the TnpB protein and DNA polymerase domain may be comprised in a fusion protein.

In some embodiments, the systems may comprise a TnpB enzyme (or variant thereof such as a dTnpB or TnpB nickase) and a DNA polymerase (e.g., phi29, T4, T7 DNA polymerase). The systems may further comprise a single-stranded DNA or double-stranded DNA template. The DNA template may comprise i) a first sequence homologous to a target site of the TnpB protein on the target polynucleotide, and/or ii) a second sequence homologous to another region of the target polynucleotide. In some embodiments, the template may be a synthetic single-stranded or PCR-generated DNA molecule, (optionally end-protected by modified nucleotides), or a viral genome (e.g., AAV). In another embodiment, the template is generated using a reverse transcriptase. When the system is delivered into a cell, an endogenous DNA polymerase in the cell may be used. Alternatively or additionally, an exogenous DNA polymerase may be expressed in the cell.

The DNA template may be end-protected by one or more modified nucleotides, or comprises a portion of a viral genome. In some embodiment, the DNA template comprises LNA or other modifications (e.g., at the 3′ end). The presence of LNA and/or the modifications may lead to more efficient annealing with the 3′ flap generated by TnpB protein cleavage.

Examples of DNA polymerase include Taq, Tne (exo-), Tma (exo-), Pfu (exo-), Pwo (exo-), Thermoanaerobacter thermohydrosulfuricus DNA polymerase, Thermococcus litoralis DNA polymerase I, E. coli DNA polymerase I, Taq DNA polymerase I, Tth DNA polymerase I, Bacillus stearothermophilus (Bst) DNA polymerase I, E. coli DNA polymerase III, bacteriophage T5 DNA polymerase, bacteriophage M2 DNA polymerase, bacteriophage T4 DNA polymerase, bacteriophage T7 DNA polymerase, bacteriophage phi29 DNA polymerase, bacteriophage PRD1 DNA polymerase, bacteriophage phi15 DNA polymerase, bacteriophage phi21DNA polymerase, bacteriophage PZE DNA polymerase, bacteriophage PZA DNA polymerase, bacteriophage Nf DNA polymerase, bacteriophage M2Y DNA polymerase, bacteriophage B103 DNA polymerase, bacteriophage SF5 DNA polymerase, bacteriophage GA-1 DNA polymerase, bacteriophage Cp-5 DNA polymerase, bacteriophage Cp-7 DNA polymerase, bacteriophage PR4 DNA polymerase, bacteriophage PR5 DNA polymerase, bacteriophage PR722 DNA polymerase and bacteriophage L17 DNA polymerase.

### TnpB Ligase Systems

In general, the systems comprise a TnpB protein and a ligase associated with the TnpB protein. The TnpB protein may be recruited to the target sequence by an ωRNA comprising a spacer capable of binding the target sequence, and generate a break on the target sequence. The ωRNA may further comprise a template sequence with desired mutations or other sequence elements. The template sequence may be ligated to the target sequence to introduce the mutations or other sequence elements to the nucleic acid molecule. The TnpB protein may be a nickase that generates a single-strand break on nucleic acid molecule, and the ligase may be a single-strand DNA ligase. In some embodiments, the systems comprise a pair of TnpB-ligases complexes with two distinct ωRNA sequences. Each TnpB-ligase complex can target one strand of a double-stranded polynucleotide, and work together to effectively modify the sequence of the double-stranded polynucleotides.

In some examples, the TnpB is associated with a ligase or functional fragment thereof. The ligase may ligate a single-strand break (a nick) generated by the TnpB. In certain cases, the ligase may ligate a double-strand break generated by the TnpB. In certain examples, the TnpB is associated with a reverse transcriptase or functional fragment thereof.

The present invention further provides systems and methods of modifying a nucleic acid sequence using a pair of distinct TnpB-ligase-ωRNA complexes, said systems and methods comprising: (a) an engineered TnpB protein connected to or complexed with a ligase; (b) two distinct ωRNA sequences complexed with such TnpB-ligase protein complex to form a first and a second distinct TnpB-ligase ωRNA complexes; (c) the first TnpB-ligase-ωRNA complex binding to one strand of a target double-stranded polynucleotide sequence, and the second TnpB-ligase-ωRNA complex binding to another strand of the target double-stranded polynucleotide sequence; (d) upon binding of the said complexes to the locus of interest the effector protein induces the modification of the sequences associated with or at the target locus of interest, whereby the two TnpB-ligase-ωRNA complexes work together on different strands of the double-stranded target sequence and modify the sequence.

One of the advantages of using such a “pair” of TnpB-ligase-ωRNA complexes includes high efficiency in modifying the sequence associated with or at the locus of interest of target double-stranded polynucleotides.

In some embodiments, the TnpB protein can be a nickase. In a preferred embodiment, a ligase is linked to the TnpB protein. The ligase can ligate the donor sequence to the target sequence. The ligase can be a single-strand DNA ligase or a double-strand DNA ligase. The ligase can be fused to the carboxyl-terminus of a TnpB protein, or to the amino-terminus of a TnpB protein.

As used herein the term “ligase” refers to an enzyme, which catalyzes the joining of breaks (e.g., double-stranded breaks or single-stranded breaks (“nicks”) between adjacent bases of nucleic acids. For example, a ligase may be an enzyme capable of forming intra- or inter-molecular covalent bonds between a 5′ phosphate group and a 3′ hydroxyl group. The term “ligate” refers to the reaction of covalently joining adjacent oligonucleotides through formation of an internucleotide linkage.

DNA ligases fall into two general categories: ATP-dependent DNA ligases (EC 6.5.1.1), and NAD (+) dependent DNA ligases (EC 6.5.1.2). NAD (+) dependent DNA ligases are found only in bacteria (and some viruses) while ATP-dependent DNA ligases are ubiquitous. The ATP-dependent DNA ligases can be divided into four classes: DNA ligase I, II, III, and IV. DNA ligase I links Okazaki fragments to form a continuous strand of DNA; DNA ligase II is an alternatively spliced form of DNA ligase III, found only in non-dividing cells; DNA ligase III is involved in base excision repair; and DNA ligase IV is involved in the repair of DNA double-strand breaks by non-homologous end joining (NHEJ). Amongst all ligases, there are two types of prokaryotic and one type of eukaryotic ligases that are particularly well suited for facilitating the blunt-ended, double-stranded DNA ligation: Prokaryotic DNA ligases (T3 and T4) and Eukaryotic DNA ligase (Ligase 1).

In some cases, the ligase is specific for double-stranded nucleic acids (e.g., dsDNA, dsRNA, RNA/DNA duplex). An example of a ligase specific for double-stranded DNA and DNA/RNA hybrids is T4 DNA ligase. In some cases, the ligase is specific for single-stranded nucleic acids (e.g., ssDNA, ssRNA). An example of such ligase is CircLigase II. In some cases, the ligase is specific for RNA/DNA duplexes. In some cases, the ligase is able to work on single-stranded, double-stranded, and/or RNA/DNA nucleic acids in any combination.

In some cases, the ligase may be a pan-ligase, which is a single ligase with the ability to ligate both DNA and RNA targets. The ligase may be specific for a target (e.g., DNA-specific or RNA-specific). In some cases, the ligase may be a dual ligase system that include DNA-specific, RNA-specific, and/or pan-ligases, in any combination.

Examples of ligases that can be used with the disclosure include T4 DNA Ligase, T3 DNA Ligase, T7 DNA Ligase, E. coli DNA Ligase, HiFi Taq DNA Ligase, 9° N™ DNA Ligase, Taq DNA Ligase, SplintR® Ligase (also known as. PBCV-1 DNA Ligase or Chlorella virus DNA Ligase), Thermostable 5′ AppDNA/RNA Ligase, T4 RNA Ligase, T4 RNA Ligase 2, T4 RNA Ligase 2 Truncated, T4 RNA Ligase 2 Truncated K227Q, T4 RNA Ligase 2, Truncated KQ, RtcB Ligase (joins single stranded RNA with a 3″-phosphate or 2′,3′-cyclic phosphate to another RNA), CircLigase II, CircLigase ssDNA Ligase, CircLigase RNA Ligase, or Ampligase® Thermostable DNA Ligas, NAD-dependent ligases including Taq DNA ligase, Thermus filiformis DNA ligase, Escherichia coli DNA ligase, Tth DNA ligase, Thermus scotoductus DNA ligase (I and II), thermostable ligase, Ampligase thermostable DNA ligase, VanC-type ligase, 9° N DNA Ligase, Tsp DNA ligase, and novel ligases discovered by bioprospecting; ATP-dependent ligases including T4 RNA ligase, T4 DNA ligase, T3 DNA ligase, T7 DNA ligase, Pfu DNA ligase, DNA ligase I, DNA ligase III, DNA ligase IV, and novel ligases discovered by bioprospecting, and wild-type, mutant isoforms, and genetically engineered variants thereof.

In some embodiments, the examples of the ligases include those used in sequencing by synthesis or sequencing by ligation reactions.

### TnpB Helitron Systems

The systems and compositions herein may comprise a TnpB polypeptide, one or more nucleic acid components, and one or more components of a helitron. The systems and compositions may be used to insert a donor polynucleotide to a target polynucleotide. The systems and compositions may further comprise a donor polynucleotide.

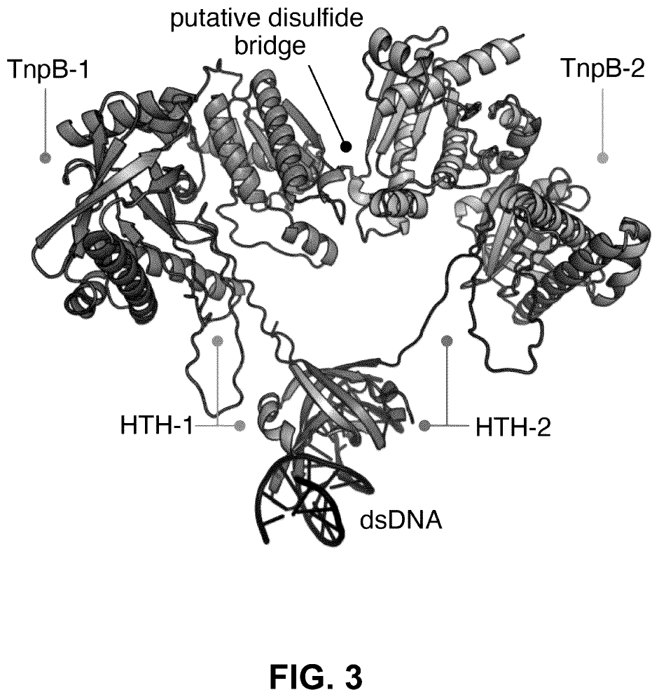

The term “helitron”, as used herein, refers to a polynucleotide (or nucleic acid segment), recognized as a transposon that captures and mobilizes gene fragments in eukaryotes. The term “helitron” as used herein refers to transposase that comprises an endonuclease domain and a C-terminal helicase domain. Helitrons are rolling-circle RNA transposons. In particular embodiments, the helitron encodes a 1400 to about 2000 amino acid, or about 1800 amino acid multidomain transposase. In embodiments, the helitron comprises a hairpin near the 3′end to function as a transposition terminator. In embodiments, the transposon comprises a RepHel motif comprising a replication initiator (Rep) and a DNA helicase (hel) domain. See, Thomas J. & Pritham E. J. Helitrons, the eukaryotic rolling-circle transposable elements. Microbiol. Spectr. 3, 893-926(2015). In embodiments, the helitron comprises a Rep nuclease domain and C-terminal helicase domain and inserts between an AT dinucleotide in single strand DNA. In an aspect, the C-terminal helicase unwinds the DNA in a 5′ to 3′ direction. The HUH nuclease domain may comprise one or two active site tyrosine residues, in embodiments, is a 2 Tyrosine (Y2) HUH endonuclease domain. Helitrons can encompass helentron, proto-helentron and helitron2 type proteins, structures of which can be as described in Thomas et al., 2015 at FIGS. 1 and 3, incorporated specifically by reference. Particular organsisms in which the helitron or helentrons have been found can include those in Table 1 of Thomas J. & Pritham E. J. Helitrons, the eukaryotic rolling-circle transposable elements. Microbiol. Spectr. 3, 893-926(2015), incorporated herein by reference. Similarly, helitrons can be identified based at least in part on the Rep motif, and conserived residues in the helitrons, and according to the alignment sequence of FIG. 2 of Thomas J. & Pritham E. J. Helitrons, the eukaryotic rolling-circle transposable elements. Microbiol. Spectr. 3, 893-926(2015), specifically incorporated herein by reference.

The expression “helitron reaction” used herein refers to a reaction wherein a transposase inserts a donor polynucleotide sequence in or adjacent to an insertion site on a target polynucleotide. The insertion site may contain a sequence or secondary structure recognized by the helitron and/or an insertion motif sequence in the target polynucleotide into which the donor polynucleotide sequence may be inserted.

As described in Grabundzija 2018, the helitron terminal sequences contains a distinct ˜150 base pairs (bp) long sequence with an absolutely conserved dinucleotide at the end of left terminal sequence (LTS), and a tetranucleotide at the end of right terminal sequence (RTS) which is preceded by a palindromic sequence that can form a hairpin structure. Grabundzija et al., Nat. Commun. 2018; 9:1278; doi: 10.1035/s41467-018-03688-w.

The helitron end sequences may be responsible for identifying the donor polynucleotide for transposition. The helitron end sequences may be the DNA sequences used to perform a transposition reaction, the end sequences may be referred to herein as right terminal sequences and left terminal sequence. The donor polynucleotide can be configured to comprise a first and second helitron recognition sequence that are at least 80%, 85%, 90%, 95% 96%, 97%, 98%, 99% or 100% complementary to a left terminal sequence and/or a right terminal sequence of a polynucleotide encoding the helitron polypeptide.

In an aspect, the palindromic sequence may be located upstream of the right terminal sequence, for example, about 5, 10, 15, 20, 25, 30, 35 nucleotides upstream of the right terminal sequence end, or about 10 to 15 nucleotides upstream of the right terminal sequence end, about 10 to 12 nucleotides or about 11 nucleotides upstream of the right terminal sequence end. Ivana Grabundzija, Nat Commun. 2016; 7:10716, doi: 10.1038/ncomms10716, incorporated herein by reference.

Exemplary helitrons can be identified using software, for example (EAHelitron) that has been used to identify Helitrons in a wide range of plant genomes. See, Hu, K., Xu, K., Wen, J. et al. Helitron distribution in Brassicaceae and whole Genome Helitron density as a character for distinguishing plant species. BMC Bioinformatics 20, 354(2019). doi: 10.1186/s12859-019-2945-8, incorporated herein by reference.

The helitron may be derived from a eukaryote. In an aspect, the helitron is derived from a mammalian genome, in an aspect, vespertilionid bats, e.g., Helibat. In embodiments, the helitron is derived from derived from a Helibat1 transposon. In embodiments, the helitron is Helraiser, the full DNA sequence of the consensus transposon, including left terminal and right terminal sequences as well as hairpin identified is provided in Grabundzija, 2016 at Supplementary FIG. 1, specifically incorporated herein by reference. In an aspect, the helitron is flanked by left and right terminal sequences of the transposon. In an aspect, the left terminal sequence and right terminal sequence terminates with the conserved 5′-TC/CTAG-3′ motif. In one embodiment, the helitron may comprise a palindromic sequence that is about 10 to about 35, or about 5-25 bp or about 19-bp-long palindromic sequence with the potential to form a hairpin structure.

Elements of these systems may be engineered to work within the context of the invention. For example, a helitron polypeptide may be fused to a polypeptide capable of generating an R-loop. Fusion may be by any appropriate linker, in an exemplary embodiment, XTEN16. The binding elements that allow a helitron polypeptide to bind, for example, the use of sequences complementary to the right terminal sequence and the left terminal sequence of the helitron may be engineered into a donor construct to facilitate entry of a donor polynucleotide sequence into a target polynucleotide.

In certain example embodiments, the Isc polypeptide, via formation of complex with a nucleic acid component sequence, directs the helitron polypeptide to a target sequence in a target polynucleotide, where the helitron facilitates integration of a donor polynucleotide sequence into the target polynucleotide.

The helitron polypeptides may also comprise one or more truncations or excisions to remove domains or regions of wild-type protein to arrive at a minimal polypeptide, alter functionality according to the system in which the helitron is used, or mutated to enhance or diminish particular activities associated with the helitron, i.e., nuclease activity or helicase activity.

### Multiplexing

In one embodiment, TnpB polypeptides may be used in a multiplex (tandem) targeting approach. For example, TnpB polypeptide herein can employ more than one nucleic acid component molecule without losing activity. This may enable the use of the TnpB polypeptide, systems or complexes as defined herein for targeting multiple DNA targets, genes or gene loci, with a single enzyme, system or complex as defined herein. The nucleic acid component molecules may be tandemly arranged, optionally separated by a nucleotide sequence such as a conserved nucleotide sequence as defined herein. The position of the different nucleic acid component molecules is the tandem does not influence the activity.

In one aspect, the TnpB polypeptides may be used for tandem or multiplex targeting. It is to be understood that any of the TnpB polypeptides, complexes, or compositions herein elsewhere may be used in such an approach. Any of the methods, products, compositions and uses as described herein elsewhere are equally applicable with the multiplex or tandem targeting approach further detailed below. By means of further guidance, the following particular aspects and embodiments are provided.

In one aspect, the invention provides for the use of a TnpB polypeptide, complex or system as defined herein for targeting multiple gene loci. In one embodiment, this can be established by using multiple (tandem or multiplex) nucleic acid component molecule sequences.

In one aspect, the invention provides methods for using one or more elements of a TnpB polypeptide, complex or system as defined herein for tandem or multiplex targeting, wherein said system herein comprises multiple nucleic acid component molecule sequences. Said sequences are separated by a nucleotide sequence, such as a conserved nucleotide sequence as defined herein elsewhere.

The TnpB polypeptides, compositions, systems or complexes as defined herein provides an effective means for modifying multiple target polynucleotides. The TnpB polypeptide, system or complex as defined herein has a wide variety of utility including modifying (e.g., deleting, inserting, translocating, inactivating, activating) one or more target polynucleotides in a multiplicity of cell types. As such the TnpB polypeptide, system or complex as defined herein of the invention has a broad spectrum of applications in, e.g., gene therapy, drug screening, disease diagnosis, and prognosis, including targeting multiple gene loci within a single system.

In one aspect, the present disclosure provides a TnpB polypeptide, system or complex as defined herein, having a TnpB polypeptide having at least one destabilization domain associated therewith, and multiple nucleic acid component molecule that target multiple nucleic acid molecules such as DNA molecules, whereby each of said multiple nucleic acid component molecules specifically targets its corresponding nucleic acid molecule, e.g., DNA molecule. Each nucleic acid molecule target, e.g., DNA molecule can encode a gene product or encompass a gene locus. Using multiple nucleic acid component molecules hence enables the targeting of multiple gene loci or multiple genes. In one embodiment the TnpB polypeptide may cleave the DNA molecule encoding the gene product. In one embodiment expression of the gene product is altered. The TnpB polypeptide and the nucleic acid component molecules do not naturally occur together. The present disclosure comprehends the nucleic acid component molecules comprising tandemly arranged nucleic acid component molecule. The present disclosure further comprehends coding sequences for the TnpB polypeptide being codon optimized for expression in a eukaryotic cell. In an embodiment the eukaryotic cell is a mammalian cell, a plant cell or a yeast cell and in a more preferred embodiment the mammalian cell is a human cell. Expression of the gene product may be decreased. The TnpB polypeptide may form part of a system or complex, which further comprises tandemly arranged nucleic acid component molecule comprising a series of 2, 3, 4, 5, 6, 7, 8, 9, 10, 15, 25, 25, 30, or more than 30 nucleic acid component molecules, each capable of specifically hybridizing to a target sequence in a genomic locus of interest in a cell. In one embodiment, the functional system or complex binds to the multiple target sequences. In one embodiment, the functional system or complex may edit the multiple target sequences, e.g., the target sequences may comprise a genomic locus, and in one embodiment, there may be an alteration of gene expression. In one embodiment, the functional system or complex may comprise further functional domains. In one embodiment, the invention provides a method for altering or modifying expression of multiple gene products. The method may comprise introducing into a cell containing said target nucleic acids, e.g., DNA molecules, or containing and expressing target nucleic acid, e.g., DNA molecules; for instance, the target nucleic acids may encode gene products or provide for expression of gene products (e.g., regulatory sequences).

In one embodiment, the TnpB polypeptide used for multiplex targeting is associated with one or more functional domains. In some more specific embodiments, the TnpB polypeptide used for multiplex targeting is a dead TnpB polypeptide. The inventors have found that the TnpB polypeptide as described herein may enable improved and/or direct access to one or more nucleotides involved in the DNA: RNA duplex.

### Inducible Systems

In one embodiment, a TnpB polypeptide may form a component of an inducible system. The inducible nature of the system would allow for spatiotemporal control of gene editing or gene expression using a form of energy. The form of energy may include but is not limited to electromagnetic radiation, sound energy, chemical energy and thermal energy. Examples of inducible system include tetracycline inducible promoters (Tet-On or Tet-Off), small molecule two-hybrid transcription activations systems (FKBP, ABA, etc.), or light inducible systems (Phytochrome, LOV domains, or cryptochrome). In one embodiment, the TnpB polypeptide may be a part of a Light Inducible Transcriptional Effector (LITE) to direct changes in transcriptional activity in a sequence-specific manner. The components of a light may include a TnpB polypeptide, a light-responsive cytochrome heterodimer (e.g., from Arabidopsis thaliana), and a transcriptional activation/repression domain. Further examples of inducible DNA binding proteins and methods for their use are provided in U.S. Provisional Application Nos. 61/736,465 and U.S. 61/721,283, and International Patent Publication No. WO 2014/018423 A2 which is hereby incorporated by reference in its entirety.

### Self-Inactivating Systems

Once all copies of a gene in the genome of a cell have been edited, continued expression of the system in that cell is no longer necessary. Indeed, sustained expression would be undesirable in case of off-target effects at unintended genomic sites, etc. Thus time-limited expression would be useful. Inducible expression offers one approach, but in addition Applicants have engineered a self-Inactivating system that relies on the use of a non-coding nucleic acid component molecule target sequence within the vector itself. Thus, after expression begins, the system will lead to its own destruction, but before destruction is complete it will have time to edit the genomic copies of the target gene (which, with a normal point mutation in a diploid cell, requires at most two edits). Simply, the self-inactivating system includes additional RNA (e.g., nucleic acid component molecule) that targets the coding sequence for the TnpB polypeptide itself or that targets one or more non-coding nucleic acid component molecule target sequences complementary to unique sequences present in one or more of the following: (a) within the promoter driving expression of the non-coding RNA elements, (b) within the promoter driving expression of the TnpB polypeptide gene, (c) within 100 bp of the ATG translational start codon in the TnpB polypeptide coding sequence, (d) within the inverted terminal repeat (iTR) of a viral delivery vector, e.g., in the AAV genome.

In some aspects, a single nucleic acid component molecule is provided that is capable of hybridization to a sequence downstream of a TnpB polypeptide start codon, whereby after a period of time there is a loss of the TnpB polypeptide expression. In some aspects, one or more nucleic acid component molecule(s) are provided that are capable of hybridization to one or more coding or non-coding regions of the polynucleotide encoding the system, whereby after a period of time there is a inactivation of one or more, or in some cases all, of the system. In some aspects of the system, and not to be limited by theory, the cell may comprise a plurality of complexes, wherein a first subset of complexes comprise a first nucleic acid component molecule capable of targeting a genomic locus or loci to be edited, and a second subset of complexes comprise at least one second nucleic acid component molecule capable of targeting the polynucleotide encoding the system, wherein the first subset of complexes mediate editing of the targeted genomic locus or loci and the second subset of complexes eventually inactivate the system, thereby inactivating further expression in the cell.

The various coding sequences (TnpB polypeptide and nucleic acid component molecule) can be included on a single vector or on multiple vectors. For instance, it is possible to encode the enzyme on one vector and the various RNA sequences on another vector, or to encode the enzyme and one nucleic acid component molecule on one vector, and the remaining nucleic acid component molecule on another vector, or any other permutation. In general, a system using a total of one or two different vectors is preferred.

Where multiple vectors are used, it is possible to deliver them in unequal numbers, and ideally with an excess of a vector which encodes the first nucleic acid component molecule relative to the second nucleic acid component molecule, thereby assisting in delaying final inactivation of the system until genome editing has had a chance to occur.

The first nucleic acid component molecule can target any target sequence of interest within a genome, as described elsewhere herein. The second nucleic acid component molecule targets a sequence within the vector which encodes the TnpB polypeptide, and thereby inactivates the enzyme's expression from that vector. Thus, the target sequence in the vector must be capable of inactivating expression. Suitable target sequences can be, for instance, near to or within the translational start codon for the TnpB polypeptide coding sequence, in a non-coding sequence in the promoter driving expression of the non-coding RNA elements, within the promoter driving expression of the TnpB polypeptide gene, within 100 bp of the ATG translational start codon in the TnpB polypeptide coding sequence, and/or within the inverted terminal repeat (iTR) of a viral delivery vector, e.g., in the AAV genome. A double stranded break near this region can induce a frame shift in the TnpB polypeptide coding sequence, causing a loss of protein expression. An alternative target sequence for the “self-inactivating” nucleic acid component molecule would aim to edit/inactivate regulatory regions/sequences needed for the expression of the system or for the stability of the vector. For instance, if the promoter for the TnpB polypeptide coding sequence is disrupted then transcription can be inhibited or prevented. Similarly, if a vector includes sequences for replication, maintenance or stability then it is possible to target these. For instance, in a AAV vector a useful target sequence is within the iTR. Other useful sequences to target can be promoter sequences, polyadenylation sites, etc.

Furthermore, if the nucleic acid component molecules are expressed in array format, the “self-inactivating” nucleic acid component molecules that target both promoters simultaneously will result in the excision of the intervening nucleotides from within the TnpB polypeptide expression construct, effectively leading to its complete inactivation. Similarly, excision of the intervening nucleotides will result where the nucleic acid component molecules target both ITRs, or targets two or more other components simultaneously. Self-inactivation as explained herein is applicable, in general, with systems in order to provide regulation of the systems. For example, self-inactivation as explained herein may be applied to the repair of mutations, for example expansion disorders, as explained herein. As a result of this self-inactivation, repair may be only transiently active.

Addition of non-targeting nucleotides to the 5′ end (e.g., 1-10 nucleotides, preferably 1-5 nucleotides) of the “self-inactivating” nucleic acid component molecule can be used to delay its processing and/or modify its efficiency as a means of ensuring editing at the targeted genomic locus prior to shut down.

In one aspect of the self-inactivating AAV system, plasmids that co-express one or more nucleic acid component molecule targeting genomic sequences of interest (e.g., 1-2, 1-5, 1-10, 1-15, 1-20, 1-30) may be established with “self-inactivating” nucleic acid component molecule that target an TnpB polypeptide sequence at or near the engineered ATG start site (e.g., within 5 nucleotides, within 15 nucleotides, within 30 nucleotides, within 50 nucleotides, within 100 nucleotides). A regulatory sequence in the U6 promoter region can also be targeted with a nucleic acid component molecule. The U6-driven nucleic acid component molecules may be designed in an array format such that multiple nucleic acid component molecule sequences can be simultaneously released. When first delivered into target tissue/cells (left cell) nucleic acid component molecules begin to accumulate while TnpB polypeptide levels rise in the nucleus. TnpB polypeptide complexes with all of the nucleic acid component molecules to mediate genome editing and self-inactivation of the TnpB polypeptide plasmids.

One aspect of a self-inactivating system is expression of singly or in tandem array format from 1 up to 4 or more different nucleic acid component sequences, e.g., up to about 20 or about 30 sequences. Each individual self-inactivating nucleic acid component molecule sequence may target a different target. Such may be processed from, e.g., one chimeric pol3 transcript. Pol3 promoters such as U6 or H1 promoters may be used. Pol2 promoters such as those mentioned throughout herein. Inverted terminal repeat (iTR) sequences may flank the Pol3 promoter-nucleic acid component molecule(s)-Pol2 promoter-TnpB polypeptide.

One aspect of a tandem array transcript is that one or more nucleic acid component molecule(s) edit the one or more target(s) while one or more self-inactivating nucleic acid component molecules inactivate the system. Thus, for example, the described system for repairing expansion disorders may be directly combined with the self-inactivating system described herein. Such a system may, for example, have two nucleic acid component molecules directed to the target region for repair as well as at least a third nucleic acid component molecule directed to self-inactivation of the TnpB polypeptide or systems.

The nucleic acid component molecule may be a control molecule. For example, it may be engineered to target a nucleic acid sequence encoding the TnpB polypeptide itself, as described in U.S. Patent Publication No. US2015232881A1, the disclosure of which is hereby incorporated by reference. In one embodiment, a system or composition may be provided with just the nucleic acid component molecule engineered to target the nucleic acid sequence encoding the TnpB polypeptide. In addition, the system or composition may be provided with the nucleic acid component molecule engineered to target the nucleic acid sequence encoding the TnpB polypeptide, as well as nucleic acid sequence encoding the TnpB polypeptide and, optionally a second nucleic acid component molecule and, further optionally, a repair template. The second nucleic acid component may be the primary target of the system or composition (such a therapeutic, diagnostic, knock out etc. as defined herein). In this way, the system or composition is self-inactivating. This is exemplified in relation to Cas in US2015232881A1 (also published as WO2015070083 (A1), and may be extrapolated to TnpB polypeptides disclosed herein, e.g., TnpB polypeptides.

### Polynucleotides Encoding TnpB Systems and Vectors

The systems herein may comprise one or more polynucleotides. The polynucleotide(s) may comprise coding sequences of components of the systems herein, e.g., TnpB polypeptide, nucleic acid component(s), functional domain(s), donor polynucleotide(s), and/or other components in the systems. The present disclosure further provides vectors or vector systems comprising one or more polynucleotides herein. The vectors or vector systems include those described in the delivery sections herein.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (snucleic acid component), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. As used herein the term “wild type” is a term of the art understood by skilled persons and means the typical form of an organism, strain, gene or characteristic as it occurs in nature as distinguished from mutant or variant forms. A “wild type” can be a base line. As used herein the term “variant” should be taken to mean the exhibition of qualities that have a pattern that deviates from what occurs in nature. The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature. “Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y. Where reference is made to a polynucleotide sequence, then complementary or partially complementary sequences are also envisaged. These are preferably capable of hybridizing to the reference sequence under highly stringent conditions. “Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence. As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this invention, it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. As used herein, “expression of a genomic locus” or “gene expression” is the process by which information from a gene is used in the synthesis of a functional gene product. The products of gene expression are often proteins, but in non-protein coding genes such as rRNA genes or tRNA genes, the product is functional RNA. The process of gene expression is used by all known life-eukaryotes (including multicellular organisms), prokaryotes (bacteria and archaea) and viruses to generate functional products to survive. As used herein, “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. The terms “polypeptide”, “peptide” and “protein” are used interchangeably herein to refer to polymers of amino acids of any length. The polymer may be linear or branched, it may comprise modified amino acids, and it may be interrupted by non amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. As used herein, the term “domain” or “protein domain” refers to a part of a protein sequence that may exist and function independently of the rest of the protein chain. As described in aspects of the invention, sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences.

In one embodiment, the polynucleotide sequence is recombinant DNA. In further embodiments, the polynucleotide sequence further comprises additional sequences as described elsewhere herein. In one embodiment, the nucleic acid sequence is synthesized in vitro.

The present disclosure provides polynucleotide molecules that encode one or more components of the system or TnpB polypeptide as referred to in any embodiment herein. In one embodiment, the polynucleotide molecules may comprise further regulatory sequences. By means of guidance and not limitation, the polynucleotide sequence can be part of an expression plasmid, a minicircle, a lentiviral vector, a retroviral vector, an adenoviral or adeno-associated viral vector, a piggyback vector, or a to 12 vector. In one embodiment, the polynucleotide sequence may be a bicistronic expression construct. In further embodiments, the isolated polynucleotide sequence may be incorporated in a cellular genome. In yet further embodiments, the isolated polynucleotide sequence may be part of a cellular genome. In further embodiments, the isolated polynucleotide sequence may be comprised in an artificial chromosome. In one embodiment, the 5′ and/or 3′ end of the isolated polynucleotide sequence may be modified to improve the stability of the sequence of actively avoid degradation. In one embodiment, the isolated polynucleotide sequence may be comprised in a bacteriophage. In other embodiments, the isolated polynucleotide sequence may be contained in agrobacterium species. In one embodiment, the isolated polynucleotide sequence is lyophilized.

### Codon Optimization

Aspects of the invention relate to polynucleotide molecules that encode one or more components of one or more systems as described in any of the embodiments herein, wherein at least one or more regions of the polynucleotide molecule may be codon optimized for expression in eukaryotic cells. In one embodiment, the polynucleotide molecules that encode one or more components of one or more systems as described in any of the embodiments herein are optimized for expression in a mammalian cell or a plant cell.

An example of a codon optimized sequence is in this instance a sequence optimized for expression in a eukaryote, e.g., humans (i.e., being optimized for expression in humans), or for another eukaryote, animal or mammal as herein discussed. In one embodiment, an enzyme coding sequence encoding a DNA/RNA-targeting TnpB polypeptide is codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In one embodiment, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In one embodiment, one or more codons (e.g., 1, 2, 3, 4, 5, 10, 15, 20, 25, 50, or more, or all codons) in a sequence encoding a TnpB polypeptide corresponds to the most frequently used codon for a particular amino acid.

### Delivery

The present disclosure also provides delivery systems for introducing components of the systems and compositions herein to cells, tissues, organs, or organisms. A delivery system may comprise one or more delivery vehicles and/or cargos. Exemplary delivery systems and methods include those described in paragraphs to of Feng Zhang et al., (WO2016106236A1), and pages 1241-1251 and Table 1 of Lino C A et al., Delivering CRISPR: a review of the challenges and approaches, DRUG DELIVERY, 2018, VOL. 25, NO. 1, 1234-1257, which are incorporated by reference herein in their entireties and can be adapted for use with the TnpB proteins disclosed herein.

In one embodiment, the delivery systems may be used to introduce the components of the systems and compositions to plant cells. For example, the components may be delivered to plant using electroporation, microinjection, aerosol beam injection of plant cell protoplasts, biolistic methods, DNA particle bombardment, and/or Agrobacterium-mediated transformation. Examples of methods and delivery systems for plants include those described in Fu et al., Transgenic Res. 2000 February; 9(1): 11-9; Klein R M, et al., Biotechnology. 1992; 24:384-6; Casas A M et al., Proc Natl Acad Sci USA. 1993 Dec. 1; 90(23): 11212-11216; and U.S. Pat. No. 5,563,055, Davey M R et al., Plant Mol Biol. 1989 September; 13(3): 273-85, which are incorporated by reference herein in their entireties.

The example delivery compositions, systems, and methods described herein related to composition or TnpB polypeptide also apply to functional domains and other components (e.g., other proteins and polynucleotides related to the TnpB polypeptide, such as reverse transcriptase, nucleotide deaminase, retrotransposon, donor polynucleotide, etc.). In a preferred embodiment, the composition comprises delivery of the polypeptides via mRNA.

### RNA Delivery

In one embodiment, the TnpB system may comprise is delivered as an mRNA encoding the TnpB polypeptide. The ωRNA may be delivered with or separately from the mRNA encoding the TnpB polypeptide. The in vivo translation efficiency of mRNA molecules may be further increased by RNA engineering. To achieve effective translation, mRNA requires five structural elements, including the 5′ cap, 3′ poly(A) tail, protein-coding sequence and 5′ and 3′ untranslated regions (UTRs) with sequence engineering of one or more of these elements may be utilized to improve translation in vivo.

In some embodiments, the isolated mRNA is not self-replicating.

In some embodiment, the isolated mRNA comprises and/or encodes one or more 5′terminal cap (or cap structure), 3′terminal cap, 5′untranslated region, 3′untranslated region, a tailing region, or any combination thereof.

In some embodiments, the capping region of the isolated mRNA region may be from 1 to 10, e.g., 2-9, 3-8, 4-7, 1-5, 5-10, or at least 2, or 10 or fewer nucleotides in length. In some embodiments, the cap is absent.

In an exemplary embodiment, mRNA can be synthesized in vitro and transferred directly into target cells, and may be further modified. For example, the mRNA may comprise a 5′ end of endogenous mRNAs modified with a 7-methylguanosine cap structure, with polyadenylated 3′ end, which may facilitate protein production. Modification of pyrimidine residues may also be performed to enhance transgene expression from delivered mRNAs, as it may lower stimulation of the innate immune system of host cells. In an example embodiment, the mRNA comprises an anti-reverse cap analog and a 120-nt poly(A) tail, and optionally may comprise cytosine and uridine residues replaced with 5-methylcytosine and pseudouridine. See, U.S. Patent Publication 2019/0151474, incorporated herein by reference.

In some embodiments, a 5′-cap structure is cap0, cap1, ARCA, inosine, N1-methyl-guanosine, 2′-fluoro-guanosine, 7-deaza-guanosine, 8-oxo-guanosine, 2-amino-guanosine, LNA-guanosine, or 2-azido-guanosine.

In some embodiments, the 5′terminal cap is 7 mG (5′) ppp (5′) NlmpNp, m7GpppG cap, N7-methylguanine. In some embodiments, the 3′terminal cap is a 3′-O-methyl-m7GpppG.

In some embodiments, the 3′-UTR is an alpha-globin 3′-UTR. In some embodiments, the 5′-UTR comprises a Kozak sequence.

In some embodiments, the tailing sequence may range from absent to 500 nucleotides in length (e.g., at least 60, 70, 80, 90, 120, 140, 160, 180, 200, 250, 300, 350, 400, 450, or 500 nucleotides). In some embodiments, the tailing region is or includes a polyA tail. Where the tailing region is a polyA tail, the length may be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional. In some embodiments, the poly-A tail is at least 160 nucleotides in length.

In some embodiments, the mRNA polynucleotide includes a stabilization element. In some embodiments, the stabilization element is a histone stem-loop. In some embodiments, the stabilization element is a nucleic acid sequence having increased GC content relative to wild type sequence.

In an embodiment, it is desirable to reduce the immunogenic sequence motifs of the mRNA for delivery. Exemplary techniques are known in the art, see, e.g., International Patent Publication WO/2020/033720, discussing exemplary immunogenic sequence motifs for removal, including those that can bind human TLR8), incorporated herein by reference.

The isolated mRNA(s) can be made in part or using only in vitro transcription. Methods of making polynucleotides by in vitro transcription are known in the art and are described in U.S. Provisional Patent Application Nos. 61/618,862, 61/681,645, 61/737,130, 61/618,866, 61/681,647, 61/737,134, 61/618,868, 61/681,648, 61/737,135, 61/618,873, 61/681,650, 61/737,147, 61/618,878, 61/681,654, 61/737,152, 61/618,885, 61/681,658, 61/737,155, 61/618,896, 61/668,157, 61/681,661, 61/737,160, 61/618,911, 61/681,667, 61/737,168, 61/618,922, 61/681,675, 61/737,174, 61/618,935, 61/681,687, 61/737,184, 61/618,945, 61/681,696, 61/737,191, 61/618,953, 61/681,704 61/737,203, International Publication Nos WO2013151666, WO2013151668, WO2013151663, WO2013151669, WO2013151670, WO2013151664, WO2013151665, WO2013151736, WO2013151672, WO2013151671 WO2013151667, and WO/2020/205793A1; the contents of each of which are herein incorporated by reference in their entireties. Cell-free production methods of making ribonucleic acid, including large scale syntheses are described, for example in U.S. Pat. No. 10,954,541, incorporated herein by reference in its entirety.

Targeted delivery of mRNA and endosomal escape are generally requirements of effective mRNA use. Lipids, including lipid nanoparticles, lipid-like materials, polymers are particularly preferred delivery vehicles, as detailed elsewhere herein.

### Cargos

The delivery systems may comprise one or more cargos. The cargos may comprise one or more components of the systems and compositions herein. A cargo may comprise one or more of the following: i) a plasmid encoding one or more proteins components in the compositions and systems such as the TnpB polypeptide and/or functional domains; ii) a plasmid encoding one or more nucleic acid components, iii) mRNA of one or more one or more proteins components in the compositions and systems such as the TnpB polypeptide and/or functional domains; iv) one or more nucleic acid component molecules; v) one or more proteins components in the compositions and systems such as the TnpB polypeptide and/or functional domains; vi) any combination thereof. The one or more protein components may include the nuclei acid-guided nuclease (e.g., Cas), reverse transcriptase, nucleotide deaminase, retrotransposon protein, other functional domain, or any combination thereof.

In some examples, a cargo may comprise a plasmid encoding one or more proteins components in the compositions and systems such as the TnpB polypeptide and/or functional domains and one or more (e.g., a plurality of) nucleic acid component molecules. In some cases, the plasmid may also encode a recombination template (e.g., for HDR). In one embodiment, a cargo may comprise mRNA encoding one or more protein components and one or more nucleic acid component molecules.

In some examples, a cargo may comprise one or more protein components and one or more nucleic acid component molecules, e.g., in the form of ribonucleoprotein complexes (RNP). The ribonucleoprotein complexes may be delivered by methods and systems herein. In some cases, the ribonucleoprotein may be delivered by way of a polypeptide-based shuttle agent. In one example, the ribonucleoprotein may be delivered using synthetic peptides comprising an endosome leakage domain (ELD) operably linked to a cell penetrating domain (CPD), to a histidine-rich domain and a CPD, e.g., as describe in WO2016161516. RNP may also be used for delivering the compositions and systems to plant cells, e.g., as described in Wu J W, et al., Nat Biotechnol. 2015 November; 33(11): 1162-4.

### Physical Delivery

In one embodiment, the cargos may be introduced to cells by physical delivery methods. Examples of physical methods include microinjection, electroporation, and hydrodynamic delivery. Both nucleic acid and proteins may be delivered using such methods. For example, one or more protein components may be prepared in vitro, isolated, (refolded, purified if needed), and introduced to cells.

### Microinjection

Microinjection of the cargo directly to cells can achieve high efficiency, e.g., above 90% or about 100%. In one embodiment, microinjection may be performed using a microscope and a needle (e.g., with 0.5-5.0 μm in diameter) to pierce a cell membrane and deliver the cargo directly to a target site within the cell. Microinjection may be used for in vitro and ex vivo delivery.

Plasmids comprising coding sequences for one or more protein components and/or nucleic acid components, mRNAs, and/or nucleic acid component molecules, may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery nucleic acid component directly to the nucleus and mRNA to the cytoplasm, e.g., facilitating translation and shuttling of one or more protein components to the nucleus.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up- or down-regulate a specific gene within the genome of a cell, e.g., using TnpB.

### Electroporation

In one embodiment, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses, non-viral vehicles, and other delivery reagents described herein.

The delivery vehicles in accordance with the present invention may have a greatest dimension (e.g., diameter) of less than 100 microns (μm). In one embodiment, the delivery vehicles have a greatest dimension of less than 10 μm. In one embodiment, the delivery vehicles may have a greatest dimension of less than 2000 nanometers (nm). In one embodiment, the delivery vehicles may have a greatest dimension of less than 1000 nanometers (nm). In one embodiment, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In one embodiment, the delivery vehicles may have a greatest dimension ranging between 25 nm and 200 nm.

In one embodiment, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles). Nanoparticles may also be used to deliver the compositions and systems to plant cells, e.g., as described in International Patent Publication No. WO 2008042156, US Publication Application No. US 20130185823, and International Patent Publication No WO 2015/089419.

### Vectors

The systems, compositions, and/or delivery systems may comprise one or more vectors. The present disclosure also includes vector systems. A vector system may comprise one or more vectors. In one embodiment, a vector refers to a nucleic acid molecule capable of transporting another nucleic acid to which it has been linked. Vectors include nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques. Certain vectors may be capable of autonomous replication in a host cell into which they are introduced (e.g., bacterial vectors having a bacterial origin of replication and episomal mammalian vectors). Some vectors (e.g., non-episomal mammalian vectors) are integrated into the genome of a host cell upon introduction into the host cell, and thereby are replicated along with the host genome. In certain examples, vectors may be expression vectors, e.g., capable of directing the expression of genes to which they are operatively-linked. In some cases, the expression vectors may be for expression in eukaryotic cells. Common expression vectors of utility in recombinant DNA techniques are often in the form of plasmids.

Examples of vectors include pGEX, pMAL, pRIT5, E. coli expression vectors (e.g., pTrc, pET 11d, yeast expression vectors (e.g., pYepSec1, pMFa, pJRY88, pYES2, and picZ, Baculovirus vectors (e.g., for expression in insect cells such as SF9 cells) (e.g., pAc series and the pVL series), mammalian expression vectors (e.g., pCDM8 and pMT2PC.

A vector may comprise i) one or more protein components encoding sequence(s), and/or ii) a single, or at least 2, at least 3, at least 4, at least 5, at least 6, at least 7, at least 8, at least 9, at least 10, at least 12, at least 14, at least 16, at least 32, at least 48, at least 50 nucleic acid component molecule(s) encoding sequences. In a single vector, there can be a promoter for each RNA coding sequence. Alternatively or additionally, in a single vector, there may be a promoter controlling (e.g., driving transcription and/or expression) multiple RNA encoding sequences.

Furthermore, that compositions or systems may be delivered via a vector, e.g., a separate vector or the same vector that is encoding the complex. When provided by a separate vector, the RNA that targets TnpB polypeptide expression can be administered sequentially or simultaneously. When administered sequentially, the RNA that targets TnpB polypeptide expression is to be delivered after the RNA that is intended for e.g., gene editing or gene engineering. This period may be a period of minutes (e.g., 5 minutes, 10 minutes, 20 minutes, 30 minutes, 45 minutes, 60 minutes). This period may be a period of hours (e.g., 2 hours, 4 hours, 6 hours, 8 hours, 12 hours, 24 hours). This period may be a period of days (e.g., 2 days, 3 days, 4 days, 7 days). This period may be a period of weeks (e.g., 2 weeks, 3 weeks, 4 weeks). This period may be a period of months (e.g., 2 months, 4 months, 8 months, 12 months). This period may be a period of years (2 years, 3 years, 4 years). In this fashion, the TnpB polypeptide associates with a first nucleic acid component molecule capable of hybridizing to a first target, such as a genomic locus or loci of interest and undertakes the function(s) desired of the system (e.g., gene engineering); and subsequently the TnpB polypeptide may then associate with the second nucleic acid component molecule capable of hybridizing to the sequence comprising at least part of the TnpB polypeptide. Where the nucleic acid component molecule targets the sequences encoding expression of the TnpB polypeptide, the enzyme becomes impeded, and the system becomes self-inactivating. In the same manner, RNA that targets TnpB polypeptide expression applied via, for example liposome, lipofection, particles, microvesicles as explained herein, may be administered sequentially or simultaneously. Similarly, self-inactivation may be used for inactivation of one or more nucleic acid component molecule used to target one or more targets.

### Regulatory Elements

A vector may comprise one or more regulatory elements. The regulatory element(s) may be operably linked to coding sequences of TnpB polypeptide, accessory proteins, nucleic acid component scaffold and/or nucleic acid component molecule or combination thereof. The term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). In certain examples, a vector may comprise: a first regulatory element operably linked to a nucleotide sequence encoding a TnpB polypeptide, and a second regulatory element operably linked to a nucleotide sequence encoding a nucleic acid component molecule.

Examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific.

Examples of promoters include one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter.

### Viral Vectors

The cargos may be delivered by viruses. In one embodiment, viral vectors are used. A viral vector may comprise virally-derived DNA or RNA sequences for packaging into a virus (e.g., retroviruses, replication defective retroviruses, adenoviruses, replication defective adenoviruses, and adeno-associated viruses). Viral vectors also include polynucleotides carried by a virus for transfection into a host cell. Viruses and viral vectors may be used for in vitro, ex vivo, and/or in vivo deliveries.

### Adeno Associated Virus (AAV)

The systems and compositions herein may be delivered by adeno associated virus (AAV). AAV vectors may be used for such delivery. AAV, of the Dependovirus genus and Parvoviridae family, is a single stranded DNA virus. In one embodiment, AAV may provide a persistent source of the provided DNA, as AAV delivered genomic material can exist indefinitely in cells, e.g., either as exogenous DNA or, with some modification, be directly integrated into the host DNA. In one embodiment, AAV do not cause or relate with any diseases in humans. The virus itself is able to efficiently infect cells while provoking little to no innate or adaptive immune response or associated toxicity.

Examples of AAV that can be used herein include AAV-1, AAV-2, AAV-3, AAV-4, AAV-5, AAV-6, AAV-8, and AAV-9. The type of AAV may be selected with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. AAV-2-based vectors were originally proposed for CFTR delivery to CF airways, other serotypes such as AAV-1, AAV-5, AAV-6, and AAV-9 exhibit improved gene transfer efficiency in a variety of models of the lung epithelium. Examples of cell types targeted by AAV are described in Grimm, D. et al, J. Virol. 82:5887-5911(2008), and shown as follows in Table 3:

TABLE 3

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

The AAV particles may be created in HEK 293 T cells. Once particles with specific tropism have been created, they are used to infect the target cell line much in the same way that native viral particles do. This may allow for persistent presence of the components in the infected cell type, and what makes this version of delivery particularly suited to cases where long-term expression is desirable. Examples of doses and formulations for AAV that can be used include those describe in U.S. Pat. Nos. 8,454,972 and 8,404,658.

Various strategies may be used for delivery the systems and compositions herein with AAVs. In some examples, coding sequences of TnpB polypeptide and nucleic acid component may be packaged directly onto one DNA plasmid vector and delivered via one AAV particle. In some examples, AAVs may be used to deliver nucleic acid components into cells that have been previously engineered to express TnpB polypeptide. In some examples, coding sequences of TnpB polypeptide and nucleic acid component may be made into two separate AAV particles, which are used for co-transfection of target cells. In some examples, markers, tags, and other sequences may be packaged in the same AAV particles as coding sequences of TnpB polypeptide and/or nucleic acid components.

### Lentiviruses

The systems and compositions herein may be delivered by lentiviruses. Lentiviral vectors may be used for such delivery. Lentiviruses are complex retroviruses that have the ability to infect and express their genes in both mitotic and post-mitotic cells.

Examples of lentiviruses include human immunodeficiency virus (HIV), which may use its envelope glycoproteins of other viruses to target a broad range of cell types; minimal non-primate lentiviral vectors based on the equine infectious anemia virus (EIAV), which may be used for ocular therapies. In one embodiment, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2: 36ra43) may be used/and or adapted to the TnpB system herein.

Lentiviruses may be pseudo-typed with other viral proteins, such as the G protein of vesicular stomatitis virus. In doing so, the cellular tropism of the lentiviruses can be altered to be as broad or narrow as desired. In some cases, to improve safety, second- and third-generation lentiviral systems may split essential genes across three plasmids, which may reduce the likelihood of accidental reconstitution of viable viral particles within cells.

In some examples, leveraging the integration ability, lentiviruses may be used to create libraries of cells comprising various genetic modifications, e.g., for screening and/or studying genes and signaling pathways.

### Adenoviruses

The systems and compositions herein may be delivered by adenoviruses. Adenoviral vectors may be used for such delivery. Adenoviruses include nonenveloped viruses with an icosahedral nucleocapsid containing a double stranded DNA genome. Adenoviruses may infect dividing and non-dividing cells. In one embodiment, adenoviruses do not integrate into the genome of host cells, which may be used for limiting off-target effects of systems in gene editing applications.

### Viral Vehicles for Delivery to Plants

The systems and compositions may be delivered to plant cells using viral vehicles. In particular embodiments, the compositions and systems may be introduced in the plant cells using a plant viral vector (e.g., as described in Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

### Non-Viral Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, gold nanoparticles, streptolysin O, multifunctional envelope-type nanodevices (MENDs), lipid-coated mesoporous silica particles, and other inorganic nanoparticles. Targeted delivery of RNA and endosomal escape are generally requirements of effective RNA use. Lipids, including lipid nanoparticles, lipid-like materials, polymers are particularly preferred delivery vehicles for RNA, as detailed further below.

### Nanoparticles

Delivery vehicles for use with the present compositions may comprise nanoparticles including lipid nanoparticles. Other particle systems, including polymer based materials such as calcium phosphate-silicate nanoparticle, a calcium phosphate nanoparticle, a silica nanoparticle, and poly(amido-amine), poly-beta amino-esters (PBAEs), and polyethylenimine (PEI) can be used. See, e.g., Trepotec et al. Mol. Therapy 27:4 Apr. 2019. In an embodiment, the exemplary nanoparticle comprises modified dendrimers comprising cores, one or more of homogeneous or heterogeneous intermediate and terminal layers for the enclosure and delivery of nucleic acid, e.g., mRNA. Modified dendrimers can preferably comprise one or more polyester dendrimers, for example, comprising a core branching into one or more generations of polyester units, with polyester attached at surface via amine linkers (e.g., polyamine) to hydrophobic units (e.g., fatty acid derivative), including polyamidoamine (PAMAM) dendrimers, polypropylene imine (PPI) dendrimers, or polyethylene imine (PEI) dendrimers. The plurality of intermediate layers may comprise both at least one layer modified for endosomal escape and a polyfluorocarbon. Exemplary molecules and methods of making can be found in WO/2020/132196, and WO 2021/207020, incorporated herein by reference. Formulas IB, II and III of International Patent Publication WO 2021/207020 are specifically incorporated herein by reference as exemplary nanoparticle delivery vehicles for the delivery of nucleic acids.

### Lipid Particles

The delivery vehicles may comprise lipid particles, e.g., lipid nanoparticles (LNPs) and liposomes. Lipidic aminoglycosides and derivatives thereof are known in the art for delivery of RNA, including dioleylamine-A-succinyl-neomycin (“DOSN”), dioleylamine-A-succinyl-paromomycin (“DOSP”), NeoCHol. NeoSucChol, ParomoChol. ParomoCapSucDOLA, ParamoLysSucDOLA, NeoDiSucDODA, NeodiLysSucDOLA, and [ParomoLys] 2-Glu-Lys-[SucDOLA] 2 as detailed in International Patent Publicaiton WO 2008/040792, incorporated herein by reference.

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of TnpB polypeptide and/or nucleic acid component) and/or RNA molecules (e.g., mRNA of TnpB polypeptide, nucleic acid component molecules). In certain cases, LNPs may be use for delivering RNP complexes of TnpB polypeptide/nucleic acid component.

Cationic lipids form complexes with mRNA to form a lipoplex which is then endocytosed by cells. In an example embodiment, the LNP comprises a cationic lipid, a helper lipid, cholesterol, and polyethylene glycol (PEG). In an example embodiment, the LNP can comprise paromomycin-based cationic lipids, with either an amide or a phosphoramide linker, and on the other hand two imidazole-based neutral lipids, having as well either an amide or a phosphoramide function as linker. In an embodiment, assemblies can be obtained when the cationic and helper lipids comprise different linkers. See, Colombani, et al., Self-assembling complexes between binary mixtures of lipids with different linkers and nucleic acids promote universal mRNA, DNA and siRNA delivery. J. Control Release. (2017) doi: 10.1016/j.jconrel.2017.01.041

In an embodiment, the nanoparticles can be developed according to selective organ targeting (SORT) wherein multiple classes of lipid nanoparticles are systematically engineered to exclusively edit extrahepatic tissues via addition of a supplemental SORT molecule. See, e.g., Cheng et al., Nature Nanotechnology 15, 313-320 2020). The approach has been shown with dendrimer lipid nanoparticles (DLNPs), stable nucleic acid lipid particles (SNALPs), and lipid-like nanoparticles (LLNPs), including with use of ionizable cationic lipids (5A2-SC8, C12-200, or DLin-MC3-DMA) 36,48,49, zwitterionic lipids (DOPE or DSPC), cholesterol, DMG-PEG, and permanently cationic lipids (DOTAP, DDAB or EPC). Wei et al., Systemic nanoparticle delivery of CRISPR-Cas9 ribonucleproteins for effective tissue specific genome editing., Nature Comm. (2020) 11:3232, doi: 10.1038/s4146020170293, incorporated herein by reference.

In one embodiment, the composition comprises a plurality of lipid nanoparticles comprising a cationic lipid, a neutral lipid, a cholesterol, a PEG lipid, or a combination thereof, wherein the plurality of lipid nanoparticles optionally has a mean particle size of between 80 nm and 160 nm; and wherein the lipid nanoparticles comprise one or more polynucleotides encoding at least one polypeptide of the present invention, e.g., TnpB polypeptide.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol) 2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-220 December 2011).

Further cationic lipids may comprise di-O-octadecenyl-3-trimethylammonium-propane, (DOTMA), 1,2-dioleoyl-sn-glycero-3-phosphoethanolamine (DOPE), 1,2-dioleoyl-3-trimethylammonium-propane (DOTAP), a biodegradable analogue of DOTMA, alone or in combination with further materials such as, for example cholesterol. Such Cationic lipid LNPs can be delivered as, for example, nanoemulsions and may further incorporate carbonate apatite (increase interaction between particles and cell membranes), or with conjugation with fibronectin, accelerating endocytosis. Other quaternary ammonium lipids, such as Dimethyldioctadecylammonium bromide (DDAB) are also 2,3-dioleyloxy-N-[2-(sperminecarboxamido) ethyl]-N,N-dimethyl-1-propanaminium trifluoroacetate (DOSPA) are also contemplated for use in delivery.

Lipid nanoparticles for mRNA delivery can comprise 2-(((((3S,8S,9S,10R,13R,14S, 17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy) carbonyl)amino)-N,N-bis(2-hydroxyethyl)-N-methylethan-1-aminium bromide (BHEM-Cholesterol). See, Zhang, Y. et al. In situ repurposing of dendritic cells with CRISPR/Cas9-based nanomedicine to induce transplant tolerance. Biomaterials 217, 119302(2019), incorporated herein by reference.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, the lipid nanoparticle is any nanoparticle described in U.S. Pat. No. 10,442,756, and/or comprises any compound described in U.S. Pat. No. 10,442,756, including but not limited to a nanoparticle according to any one of Formulas (IA) or (II) described therein.

In some embodiments, the lipid nanoparticle is any nanoparticle described in e.g., U.S. Pat. No. 10,266,485, and/or comprises any compound described in U.S. Pat. No. 10,266,485, including but not limited to a nanoparticle according to Formula (II) described therein.

In some embodiments, the lipid nanoparticle is a nanoparticle described in U.S. Pat. No. 9,868,692, and/or comprises a compound described in e.g., U.S. Pat. No. 9,868,692, including but not limited to a nanoparticle according to Formula (I), (IA), (II), (IIa), (IIb), (IIc), (IId), (IIe),

In some embodiments, a lipid nanoparticle comprises compounds of Formula (I) and/or Formula (II) as described in U.S. patent Ser. No. 10/272,150.

In some embodiments, the mRNA is formulated in a lipid nanoparticle that comprises a compound selected from Compounds 3, 18, 20, 25, 26, 29, 30, 60, 108-112 and 122 of U.S. Pat. No. 10,272,150.

In some embodiments, at least 80% (e.g., 85%, 90%, 95%, 98%, 99%) of the uracil in the open reading frame have a chemical modification, optionally wherein the vaccine is formulated in a lipid nanoparticle (e.g., a lipid nanoparticle comprises a cationic lipid, a PEG-modified lipid, a sterol and a non-cationic lipid).

In some embodiments, the lipid nanoparticle has a mean diameter of 50-200 nm.

In some embodiments, a lipid nanoparticle comprises Compounds 3, 18, 20, 25, 26, 29, 30, 60, 108-112, or 122 as set forth in U.S. patent Ser. No. 10/272,150.

In some embodiments, the lipid nanoparticle has a polydispersity value of less than 0.4 (e.g., less than 0.3, 0.2 or 0.1).

In some embodiments, a plurality of lipid nanoparticles, such as when contained in a formulation, has a mean PDI of between 0.02 and 0.2. In some embodiments, a plurality of lipid nanoparticles, such as when contained in a formulation comprising one or more polynucleotide(s), has a mean lipid to polynucleotide ratio (wt/wt) of between 10 and 20.

In some embodiments, the lipid nanoparticle has a net neutral charge at a neutral pH value.

### Liposomes

In one embodiment, a lipid particle may be liposome. Liposomes are spherical vesicle structures composed of a uni- or multilamellar lipid bilayer surrounding internal aqueous compartments and a relatively impermeable outer lipophilic phospholipid bilayer. In one embodiment, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In one embodiment, the liposome comprises a transport polymer, which may optionally be branched, comprising at least 10 amino acids and a ratio of histidine to non-histidine amino acids greater than 1.5 and less than 10. The branched transport polymer can comprise one or more backbones, one or more terminal branches, and optionally, one or more non-terminal branches. See, U.S. Pat. No. 7,070,807, incorporated herein by reference in its entirety. In one embodiment, the transposrt polymer is a Histidine-Lysine co-polymer (HKP) used to package and deliver mRNA and other cargos. See, U.S. Pat. Nos. 7,163,695, and 7,772,201, incorporated herein by reference in their entireties.

### Stable Nucleic-Acid-Lipid Particles (SNALPs)

In one embodiment, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol) 2000) carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-CDMA, and 1,2-dilinoleyloxy-3-(N;N-dimethyl)aminopropane (DLinDMA)

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

### Lipoplexes/Polyplexes

In one embodiment, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2b (e.g., forming DNA/Ca²⁺ microcomplexes), polyethenimine (PEI) (e.g., branched PEI), and poly(L-lysine) (PLL). Core-shell structured lipoplyplex delivery platforms can also be used and are one preferred delivery for mRNA, particularly because the core-shell structured particle can protein and gradually release mRNA upon degradation of the polymers. See, U.S. Patent Publication 2018/0360756, incorporated herein by reference.

### Cell Penetrating Peptides

In one embodiment, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the TnpB polypeptide directly, which is then complexed with the nucleic acid component and delivered to cells. In some examples, separate delivery of CPP-TnpB and CPP-nucleic acid component to multiple cells may be performed. CPP may also be used to delivery RNPs.

CPPs may be used to deliver the compositions and systems to plants. In some examples, CPPs may be used to deliver the components to plant protoplasts, which are then regenerated to plant cells and further to plants.

### DNA Nanoclews

In one embodiment, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42): 14722-5; and Sun W et al, Angew Chem Int Ed Engl. 2015 Oct. 5; 54(41): 12029-33. DNA nanoclew may have a palindromic sequences to be partially complementary to the nucleic acid component molecule within the TnpB polypeptide: nucleic acid component ribonucleoprotein complex. A DNA nanoclew may be coated, e.g., coated with PEI to induce endosomal escape.

### Gold Nanoparticles

In one embodiment, the delivery vehicles comprise gold nanoparticles (also referred to AuNPs or colloidal gold). Gold nanoparticles may form complex with cargos, e.g., TnpB polypeptide: nucleic acid component RNP. Gold nanoparticles may be coated, e.g., coated in a silicate and an endosomal disruptive polymer, PAsp (DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901.

iTOP

In one embodiment, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In one embodiment, the delivery vehicles may comprise polymer-based particles (e.g., nanoparticles). In one embodiment, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or snucleic acid component, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In one embodiment, the polymer-based particles may comprise alkylated and carboxyalkylated branched polyethylenimine. In some examples, the polymer-based particles are VIROMER, e.g., VIROMER RNAi, VIROMER RED, VIROMER mRNA. Example methods of delivering the systems and compositions herein include those described in Bawage S S et al., Synthetic mRNA expressed Cas13a mitigates RNA virus infections, biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data., doi: 10.13140/RG.2.2.23912.16642.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6: e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In one embodiment, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1): 9-21; El-Andaloussi S, et al., Nat Protoc. 2012 December; 7(12): 2112-26; Uno Y, et al., Hum Gene Ther. 2011 June; 22(6): 711-9; Zou W, et al., Hum Gene Ther. 2011 April; 22(4): 465-75. Exemplary exosomes can be generated from 293F cells, with mRNA-loaded exosomes driving higher mRNA expression than mRNA loaded LNPs in some instances. See, e.g., J. Biol. Chem. (2021) 297(5) 101266.

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

### Retrovirus Like Delivery Systems

The delivery vehicle may comprise a retro-virus like protein, such as PEG10, which is capable of incorporating a cargo into a virus-like particle. As such systems can be re-programmed to package specific cargos, polynucleotides encoding components of the TnpB systems disclosed herein may be further modified with a recognition sequence that leads to selective packaging of the TnpB components into such retro-virus like VLPs. Said VLPs may be further modified with fusogenic proteins that impart tissue or cell specificity. Example systems are disclosed in Segel et al. Mammalian retrovirus-like protein PEG10 packages its own mRNA and can be pseudotyped for mRNA delivery. 373 Science, 882-889(2021), which is incorporated herein by reference. The harnessing of natural proteins that formvirus-like particles and can deliver mRNA cargo, or Selective Endogenous eNcapsidation for cellular Delivery (SEND), may reduce immunogenic response compared to other delivery approaches.

### Genetically Modified Cells and Organisms

The present disclosure further provides cells comprising one or more components of the compositions and systems herein, e.g., the TnpB polypeptide and/or nucleic acid component(s). Also provided include cells modified by the systems and methods herein, and cell cultures, tissues, organs, organism comprising such cells or progeny thereof. In one embodiment, the present disclosure provides a method of modifying a cell or organism. The cell may be a prokaryotic cell or a eukaryotic cell. The cell may be a mammalian cell. The mammalian cell many be a non-human primate, bovine, porcine, rodent or mouse cell. The cell may be a non-mammalian eukaryotic cell such as poultry, fish or shrimp. The cell may be a therapeutic T cell or antibody-producing B-cell. The cell may also be a plant cell. The plant cell may be of a crop plant such as cassava, corn, sorghum, wheat, or rice. The plant cell may also be of an algae, tree or vegetable. The modification introduced to the cell by the present invention may be such that the cell and progeny of the cell are altered for improved production of biologic products such as an antibody, starch, alcohol or other desired cellular output. The modification introduced to the cell by the present invention may be such that the cell and progeny of the cell include an alteration that changes the biologic product produced.

In one embodiment, one or more polynucleotide molecules, vectors, or vector systems driving expression of one or more elements of the compositions, systems, or delivery systems comprising one or more elements of the TnpB system are introduced into a host cell such that expression of the elements of the TnpB system direct formation of a TnpB-targeting complex at one or more target sites. In one embodiment of the invention the host cell may be a eukaryotic cell, a prokaryotic cell, or a plant cell.

In particular embodiments, the host cell is a cell of a cell line. Cell lines are available from a variety of sources known to those with skill in the art (see, e.g., the American Type Culture Collection (ATCC) (Manassas, Va.)). In one embodiment, a cell transfected with one or more vectors described herein is used to establish a new cell line comprising one or more vector-derived sequences. In one embodiment, a cell transiently transfected with the components of a system as described herein (such as by transient transfection of one or more vectors, or transfection with RNA), and modified through the activity of a complex, is used to establish a new cell line comprising cells containing the modification but lacking any other exogenous sequence. In one embodiment, cells transiently or non-transiently transfected with one or more vectors described herein, or cell lines derived from such cells are used in assessing one or more test compounds.

Further intended are isolated human cells or tissues, plants or non-human animals comprising one or more of the polynucleotide molecules, vectors, vector systems, or cells described in any of the embodiments herein. In an aspect, host cells and cell lines modified by or comprising the compositions, systems or modified enzymes of present invention are provided, including (isolated) stem cells, and progeny thereof.

In one embodiment, the plants or non-human animals comprise at least one of the system components, polynucleotide molecules, vectors, vector systems, or cells described in any of the embodiments herein at least one tissue type of the plant or non-human animal. In one embodiment, non-human animals comprise at least one of the system components, polynucleotide molecules, vectors, vector systems, or cells described in any of the embodiments herein in at least one tissue type. In one embodiment, the presence of the system components is transient, in that they are degraded over time. In one embodiment, expression of the components of the systems and compositions described in any of the embodiments comprised in polynucleotide molecules, vectors, vector systems, or cells is limited to certain tissue types or regions in the plant or non-human animal. In one embodiment, the expression of the components of the systems and compositions described in any of the embodiments comprised in polynucleotide molecules, vectors, vector systems, or cells is dependent of a physiological cue. In one embodiment, expression of the components of the systems and compositions described in any of the embodiments comprised in polynucleotide molecules, vectors, vector systems, or cells may be triggered by an exogenous molecule. In one embodiment, expression of the components of the systems and compositions described in any of the embodiments comprised in polynucleotide molecules, vectors, vector systems, or cells is dependent on the expression of a non-TnpB molecule in the plant or non-human animal.

### Applications and Uses in General

The systems, the vector systems, the vectors and the compositions described herein may be used in various nucleic acids-targeting applications, altering or modifying synthesis of a gene product, such as a protein, nucleic acids cleavage, nucleic acids editing, nucleic acids splicing; trafficking of target nucleic acids, tracing of target nucleic acids, isolation of target nucleic acids, visualization of target nucleic acids, etc.

Aspects of the invention thus also encompass methods and uses of the compositions and systems described herein in genome engineering, e.g., for altering or manipulating the expression of one or more genes or the one or more gene products, in prokaryotic or eukaryotic cells, in vitro, in vivo or ex vivo. In some examples, the target polynucleotides are target sequences within genomic DNA, including nuclear genomic DNA, mitochondrial DNA, or chloroplast DNA.

Typically, in the context of a TnpB system, formation of a TnpB complex (comprising a nucleic acid component molecule (ωRNA) hybridized to a target sequence and complexed with one or more nucleic acid-targeting effector proteins) results in cleavage of one or both DNA or RNA strands in or near (e.g., within 1, 2, 3, 4, 5, 6, 7, 8, 9, 10, 20, 50, or more base pairs from) the target sequence. As used herein the term “sequence(s) associated with a target locus of interest” refers to sequences near the vicinity of the target sequence (e.g., within 1, 2, 3, 4, 5, 6, 7, 8, 9, 10, 20, 50, or more base pairs from the target sequence, wherein the target sequence is comprised within a target locus of interest).

In one embodiment, the present disclosure provides a method of targeting a polynucleotide, comprising contacting a sample (such as cell, population of cells, tissue, organ, or an organism) that comprises a target polynucleotide with the composition, systems, polynucleotide(s), or vector(s). The contacting may result in modification of a gene product or modification of the amount or expression of a gene product. In some examples, the target sequence of the polynucleotide is a disease-associated target sequence.

In one embodiment, the present disclosure provides a method of modifying target polynucleotides comprising delivering the composition, the one or more polynucleotides of 2, or one or more vectors to a cell or population of cells comprising the target polynucleotides, wherein the complex directs the reverse transcriptase to the target sequence and the reverse transcriptase facilitates insertion of the donor sequence from the nucleic acid component into the target polynucleotide.

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

The target polynucleotide of a complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA). Without wishing to be bound by theory, it is believed that the target sequence should be associated with a TAM (targeted adjacent motif); that is, a short sequence recognized by the complex. The precise sequence and length requirements for the TAMdiffer depending on the TnpB polypeptide used, but TAMs are typically 2-5 base pair sequences adjacent the protospacer (that is, the target sequence). TAM specificity can be determined, for example according to the experimental setup described in FIG. 8. In one embodiment, the TAMsequence comprises TCA. In embodiments, the TAMsequence is TCAN, wherein N may comprise any nucleotide. In one embodiment the TAMsequence comprises TCAG or TCAT. A skilled person will be able to identify further TAMsequences for use with a given TnpB polypeptide. Further, engineering of the TAMInteracting (PI) domain may allow programing of TAMspecificity, improve target site recognition fidelity, and increase the versatility of the TnpB polypeptide, genome engineering platform. TnpB polypeptide may be engineered to alter their TAMspecificity, for example as described in Kleinstiver B P et al. Engineered CRISPR-Cas9 nucleases with altered TAMspecificities. Nature. 2015 Jul. 23; 523(7561): 481-5. doi: 10.1038/nature14592.

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

Aspects of the invention relate to a method of targeting a polynucleotide, comprising contacting a sample that comprises the polynucleotide with a composition, system or TnpB polypeptide as described in any embodiment herein, a delivery system comprising a composition, system or TnpB polypeptide as described in any embodiment herein, a polynucleotide comprising a composition, system or TnpB polypeptide as described in any embodiment herein, a vector comprising a composition, system or TnpB polypeptide as described in any embodiment herein, or a vector system comprising a composition, system or TnpB polypeptide as described in any embodiment herein. In one embodiment, a target polynucleotide is contacted with at least two different composition, system or TnpB polypeptides. In further embodiments, the two different TnpB polypeptide have different target polynucleotide specificities, or degrees of specificity. In one embodiment, the two different TnpB polypeptide have a different TAMspecificity.

Also envisaged are methods of targeting a polynucleotide, comprising contacting a sample that comprises the polynucleotide with the composition and systems, vectors, polynucleotides, herein wherein contacting results in modification of a gene product or modification of the amount or expression of a gene product. In one embodiment, the expression of the targeted gene product is increased by the method. In one embodiment, the expression of the targeted gene product is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, p at least 90%, at least 95%, 100%. In one embodiment, the expression of the targeted gene product is increased at least 1.5-fold, at least 2-fold, at least 2.5-fold, at least 3-fold, at least 3.5-fold, at least 3.5-fold, at least 4-fold, at least 4.5-fold, at least 5-fold, at least 10-fold, at least 10-fold, at least 15-fold, at least 20-fold, at least 25-fold, at least 50-fold, at least 100-fold. In one embodiment, the expression of the targeted gene product is reduced by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%. In one embodiment, the expression of the targeted gene product is reduced at least 1.5-fold, at least 2-fold, at least 2.5-fold, at least 3-fold, at least 3.5-fold, at least 3.5-fold, at least 4-fold, at least 4.5-fold, at least 5-fold, at least 10-fold, at least 10-fold, at least 15-fold, at least 20-fold, at least 25-fold, at least 50-fold, at least 100-fold. In alternative embodiments, the expression of the targeted gene product is reduced by the method. In further embodiments, expression of the targeted gene may be completely eliminated, or may be considered eliminated as remnant expression levels of the targeted gene fall below the detection limit of methods known in the art that are used to quantify, detect, or monitor expression levels of genes.

In one embodiment, one or more polynucleotide molecules, vectors, or vector systems driving expression of one or more elements of a TnpB system or delivery systems comprising one or more elements of the TnpB system are introduced into a host cell such that expression of the elements of the TnpB system direct formation of a TnpB-targeting complex at one or more target sites. In one embodiment of the invention the host cell may be a eukaryotic cell, a prokaryotic cell, or a plant cell.

In particular embodiments, the host cell is a cell of a cell line. Cell lines are available from a variety of sources known to those with skill in the art (see, e.g., the American Type Culture Collection (ATCC) (Manassas, Va.)). In one embodiment, a cell transfected with one or more vectors described herein is used to establish a new cell line comprising one or more vector-derived sequences. In one embodiment, a cell transiently transfected with the components of a composition or system as described herein (such as by transient transfection of one or more vectors, or transfection with RNA), and modified through the activity of a complex, is used to establish a new cell line comprising cells containing the modification but lacking any other exogenous sequence. In one embodiment, cells transiently or non-transiently transfected with one or more vectors described herein, or cell lines derived from such cells are used in assessing one or more test compounds.

Further intended are isolated human cells or tissues, plants or non-human animals comprising one or more of the polynucleotide molecules, vectors, vector systems, or cells described in any of the embodiments herein. In an aspect, host cells and cell lines modified by or comprising the compositions, systems or modified enzymes of present invention are provided, including (isolated) stem cells, and progeny thereof.

In one embodiment, the plants or non-human animals comprise at least one of the compositions, polynucleotide molecules, vectors, vector systems, or cells described in any of the embodiments herein at least one tissue type of the plant or non-human animal. In certain embodiment, non-human animals comprise at least one of the compositions, polynucleotide molecules, vectors, vector systems, or cells described in any of the embodiments herein in at least one tissue type. In one embodiment, the presence of the compositions is transient, in that they are degraded over time. In one embodiment, expression of the compositions described in any of the embodiments comprised in polynucleotide molecules, vectors, vector systems, or cells is limited to certain tissue types or regions in the plant or non-human animal. In one embodiment, the expression of the compositions described in any of the embodiments comprised in polynucleotide molecules, vectors, vector systems, or cells is dependent of a physiological cue. In one embodiment, expression of the compositions described in any of the embodiments comprised in polynucleotide molecules, vectors, vector systems, or cells may be triggered by an exogenous molecule. In one embodiment, expression of the compositions described in any of the embodiments comprised in polynucleotide molecules, vectors, vector systems, or cells is dependent on the expression of a non-Cas molecule in the plant or non-human animal.

In one aspect, the invention provides methods for using one or more elements of a TnpB system. The TnpB-targeting complex of the invention provides an effective means for modifying a target DNA or RNA (single or double stranded, linear or super-coiled). The TnpB-targeting complex of the invention has a wide variety of utility including modifying (e.g., deleting, inserting, translocating, inactivating, activating) a target DNA or RNA in a multiplicity of cell types. As such, the TnpB-targeting complex of the invention has a broad spectrum of applications in, e.g., gene therapy, drug screening, disease diagnosis, and prognosis. An exemplary TnpB-targeting complex comprises a DNA or RNA-targeting effector protein complexed with a nucleic acid component molecule hybridized to a target sequence within the target locus of interest.

In one embodiment, this invention provides a method of cleaving a target polynucleotide. The method may comprise modifying a target polynucleotide using a TnpB-targeting complex that binds to the target polynucleotide and effect cleavage of said target polynucleotide. In an embodiment, the TnpB-targeting complex of the invention, when introduced into a cell, may create a break (e.g., a single or a double strand break) in the polynucleotide sequence. For example, the method can be used to cleave a disease polynucleotide in a cell. For example, an exogenous template comprising a sequence to be integrated flanked by an upstream sequence and a downstream sequence may be introduced into a cell. The upstream and downstream sequences share sequence similarity with either side of the site of integration in the polynucleotide. The exogenous template comprises a sequence to be integrated (e.g., a mutated RNA). The sequence for integration may be a sequence endogenous or exogenous to the cell. Examples of a sequence to be integrated include polynucleotide encoding a protein or a non-coding RNA (e.g., a microRNA). Thus, the sequence for integration may be operably linked to an appropriate control sequence or sequences. Alternatively, the sequence to be integrated may provide a regulatory function. The upstream and downstream sequences in the recombination template are selected to promote recombination between the RNA sequence of interest and the recombination. The upstream sequence is a polynucleotide sequence that shares sequence similarity with the sequence upstream of the targeted site for integration. Similarly, the downstream sequence is a polynucleotide sequence that shares sequence similarity with the polynucleotide sequence downstream of the targeted site of integration. The upstream and downstream sequences in the recombination template can have 75%, 80%, 85%, 90%, 95%, or 100% sequence identity with the targeted sequence. Preferably, the upstream and downstream sequences in the recombination template have about 95%, 96%, 97%, 98%, 99%, or 100% sequence identity with the targeted sequence. In some methods, the upstream and downstream sequences in the recombination template have about 99% or 100% sequence identity with the targeted sequence. An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000 bp. In some methods, the recombination template may further comprise a marker. Such a marker may make it easy to screen for targeted integrations. Examples of suitable markers include restriction sites, fluorescent proteins, or selectable markers. The recombination template of the invention can be constructed using recombinant techniques (see, for example, Sambrook et al., 2001 and Ausubel et al., 1996). In a method for modifying a target sequence by integrating a recombination template, a break (e.g., double or single stranded break in double or single stranded DNA or RNA) is introduced into the DNA or RNA sequence by the TnpB-targeting complex, the break is repaired via homologous recombination with an recombination template such that the template is integrated into the target. The presence of a double-stranded break facilitates integration of the template. In other embodiments, this invention provides a method of modifying expression of an RNA in a eukaryotic cell. The method comprises increasing or decreasing expression of a target polynucleotide by using a TnpB-targeting complex that binds to the DNA or RNA (e.g., mRNA or pre-mRNA). In some methods, a target can be inactivated to affect the modification of the expression in a cell. For example, upon the binding of a TnpB-targeting complex to a target sequence in a cell, the target is inactivated such that the sequence is not translated, the coded protein is not produced, or the sequence does not function as the wild-type sequence does. For example, a protein or microRNA coding sequence may be inactivated such that the protein or microRNA or pre-microRNA transcript is not produced. The target of a TnpB-targeting complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., ncRNA, lncRNA, tRNA, or rRNA). Examples of target RNA include a sequence associated with a signaling biochemical pathway, e.g., a signaling biochemical pathway-associated polynucleotide. Examples of target polynucleotide include a disease associated polynucleotide. A “disease-associated” polynucleotide refers to any polynucleotide which is yielding translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated polynucleotide also refers to a gene possessing mutation(s) or genetic variation that is directly responsible or is in linkage disequilibrium with a gene(s) that is responsible for the etiology of a disease. The translated products may be known or unknown, and may be at a normal or abnormal level. The target RNA of a TnpB-targeting complex can be any polynucleotide endogenous or exogenous to the eukaryotic cell. For example, the target RNA can be an RNA residing in the nucleus of the eukaryotic cell. The target polynucleotide can be a sequence coding a gene product (e.g., a protein) or a non-coding sequence (e.g., ncRNA, lncRNA, tRNA, or rRNA).

In one embodiment, the method may comprise allowing compositions to bind to the target DNA or RNA to effect cleavage of said target DNA or RNA thereby modifying the target DNA or RNA, wherein the TnpB-targeting complex comprises a nucleic acid-targeting effector protein complexed with a nucleic acid component molecule hybridized to a target sequence within said target DNA or RNA. In one aspect, the invention provides a method of modifying expression of DNA or RNA in a eukaryotic cell. In one embodiment, the method comprises allowing a TnpB-targeting complex to bind to the DNA or RNA such that said binding results in increased or decreased expression of said DNA or RNA; wherein the TnpB-targeting complex comprises a nucleic acid-targeting effector protein complexed with a nucleic acid component molecule. Similar considerations and conditions apply as above for methods of modifying a target DNA or RNA. In fact, these sampling, culturing and re-introduction options apply across the aspects of the present invention. In one aspect, the invention provides for methods of modifying a target DNA or RNA in a eukaryotic cell, which may be in vivo, ex vivo or in vitro. In one embodiment, the method comprises sampling a cell or population of cells from a human or non-human animal, and modifying the cell or cells. Culturing may occur at any stage ex vivo. The cell or cells may even be re-introduced into the non-human animal or plant. For re-introduced cells it is particularly preferred that the cells are stem cells. The compositions as described in any embodiment herein may be used to detect nucleic acid identifiers. Nucleic acid identifiers are non-coding nucleic acids that may be used to identify a particular article. Example nucleic acid identifiers, such as DNA watermarks, are described in Heider and Barnekow. “DNA watermarks: A proof of concept” BMC Molecular Biology 9:40(2008). The nucleic acid identifiers may also be a nucleic acid barcode. A nucleic-acid based barcode is a short sequence of nucleotides (for example, DNA, RNA, or combinations thereof) that is used as an identifier for an associated molecule, such as a target molecule and/or target nucleic acid. A nucleic acid barcode can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. One or more nucleic acid barcodes can be attached, or “tagged,” to a target molecule and/or target nucleic acid. This attachment can be direct (for example, covalent or non-covalent binding of the barcode to the target molecule) or indirect (for example, via an additional molecule, for example, a specific binding agent, such as an antibody (or other protein) or a barcode receiving adaptor (or other nucleic acid molecule). Target molecule and/or target nucleic acids can be labeled with multiple nucleic acid barcodes in combinatorial fashion, such as a nucleic acid barcode concatemer. Typically, a nucleic acid barcode is used to identify target molecules and/or target nucleic acids as being from a particular compartment (for example a discrete volume), having a particular physical property (for example, affinity, length, sequence, etc.), or having been subject to certain treatment conditions. Target molecule and/or target nucleic acid can be associated with multiple nucleic acid barcodes to provide information about all of these features (and more). Methods of generating nucleic acid-barcodes are disclosed, for example, in International Patent Application Publication No. WO/2014/047561.

In an embodiment, compositions induce a double strand break for the purpose of inducing HDR-mediated correction. In a further embodiment, two or more nucleic acid component molecules complexing with TnpB polypeptide or an ortholog or homolog thereof, may be used to induce multiplexed breaks for purpose of inducing HDR-mediated correction.

A recombination template nucleic acid, as that term is used herein, refers to a nucleic acid sequence which can be used in conjunction with compositions discloser herein to alter the structure of a target position. In an embodiment, the target nucleic acid is modified to have some or all of the sequence of the recombination template nucleic acid, typically at or near cleavage site(s). In an embodiment, the recombination template nucleic acid is single stranded. In an alternate embodiment, the recombination template nucleic acid is double stranded. In an embodiment, the recombination template nucleic acid is DNA, e.g., double stranded DNA. In an alternate embodiment, the recombination template nucleic acid is single stranded DNA.

In one embodiment, a recombination template is provided to serve as a template in homologous recombination, such as within or near a target sequence nicked or cleaved by a nucleic acid-targeting effector protein as a part of a TnpB-targeting complex.

A recombination template may be a component of another vector as described herein, contained in a separate vector, or provided as a separate polynucleotide. A recombination template polynucleotide may be of any suitable length, such as about or more than about 10, 15, 20, 25, 50, 75, 100, 150, 200, 500, 1000, or more nucleotides in length. In one embodiment, the recombination template polynucleotide is complementary to a portion of a polynucleotide comprising the target sequence. When optimally aligned, a recombination template polynucleotide might overlap with one or more nucleotides of a target sequences (e.g., about or more than about 1, 5, 10, 15, 20, 25, 30, 35, 40, 45, 50, 60, 70, 80, 90, 100 or more nucleotides). In one embodiment, when a recombination template sequence and a polynucleotide comprising a target sequence are optimally aligned, the nearest nucleotide of the recombination template polynucleotide is within about 1, 5, 10, 15, 20, 25, 50, 75, 100, 200, 300, 400, 500, 1000, 5000, 10000, or more nucleotides from the target sequence.

In an embodiment, the recombination template nucleic acid alters the structure of the target position by participating in homologous recombination. In an embodiment, the recombination template nucleic acid alters the sequence of the target position. In an embodiment, the recombination template nucleic acid results in the incorporation of a modified, or non-naturally occurring base into the target nucleic acid.

The recombination template sequence may undergo a breakage mediated or catalyzed recombination with the target sequence. In an embodiment, the recombination template nucleic acid may include sequence that corresponds to a site on the target sequence that is cleaved by an TnpB polypeptide mediated cleavage event. In an embodiment, the recombination template nucleic acid may include sequence that corresponds to both, a first site on the target sequence that is cleaved in a first TnpB polypeptide mediated event and a second site on the target sequence that is cleaved in a second TnpB polypeptide mediated event.

In one embodiment, the recombination template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In one embodiment, the recombination template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A recombination template nucleic acid having homology with a target position in a target gene may be used to alter the structure of a target sequence. The recombination template sequence may be used to alter an unwanted structure, e.g., an unwanted or mutant nucleotide. The recombination template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The recombination template nucleic acid may include sequence which results in: a change in sequence of 1, 2, 3, 4, 5, 6, 7, 8, 9, 10, 11, 12 or more nucleotides of the target sequence. In an embodiment, the recombination template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, or 220+/−10 nucleotides in length. In an embodiment, the recombination template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, or 220+/−20 nucleotides in length. In an embodiment, the recombination template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

A recombination template nucleic acid comprises the following components: [5′ homology arm]-[replacement sequence]-[3′ homology arm]. The homology arms provide for recombination into the chromosome, thus replacing the undesired element, e.g., a mutation or signature, with the replacement sequence. In an embodiment, the homology arms flank the most distal cleavage sites. In an embodiment, the 3′ end of the 5′ homology arm is the position next to the 5′ end of the replacement sequence. In an embodiment, the 5′ homology arm can extend at least 10, 20, 30, 40, 50, 100, 200, 300, 400, 500, 600, 700, 800, 900, 1000, 1500, or 2000 nucleotides 5′ from the 5′ end of the replacement sequence. In an embodiment, the 5′ end of the 3′ homology arm is the position next to the 3′ end of the replacement sequence. In an embodiment, the 3′ homology arm can extend at least 10, 20, 30, 40, 50, 100, 200, 300, 400, 500, 600, 700, 800, 900, 1000, 1500, or 2000 nucleotides 3′ from the 3′ end of the replacement sequence.

In one embodiment, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In one embodiment, both the 5′ and the 3′ homology arms may be shortened to avoid including certain sequence repeat elements.

In one embodiment, a recombination template nucleic acids for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Unlike TnpB polypeptide-mediated gene knockout, which permanently eliminates expression by mutating the gene at the DNA level, TnpB polypeptide knockdown allows for temporary reduction of gene expression through the use of artificial transcription factors. Mutating key residues in both DNA cleavage domains of the TnpB polypeptide, results in the generation of a catalytically inactive TnpB polypeptide. A catalytically inactive TnpB polypeptide complexes with a nucleic acid component molecule and localizes to the DNA sequence specified by that nucleic acid component molecule's targeting domain, however, it does not cleave the target DNA. Fusion of the inactive TnpB polypeptide protein to an effector domain, e.g., a transcription repression domain, enables recruitment of the effector to any DNA site specified by the nucleic acid component molecule. In one embodiment, TnpB polypeptide may be fused to a transcriptional repression domain and recruited to the promoter region of a gene. Especially for gene repression, it is contemplated herein that blocking the binding site of an endogenous transcription factor would aid in downregulating gene expression. In another embodiment, an inactive TnpB polypeptide can be fused to a chromatin modifying protein. Altering chromatin status can result in decreased expression of the target gene.

In an embodiment, a nucleic acid component molecule can be targeted to a known transcription response elements (e.g., promoters, enhancers, etc.), known upstream activating sequences, and/or sequences of unknown or known function that are suspected of being able to control expression of the target DNA.

In some methods, a target polynucleotide can be inactivated to affect the modification of the expression in a cell. For example, upon the binding of a composition to a target sequence in a cell, the target polynucleotide is inactivated such that the sequence is not transcribed, the coded protein is not produced, or the sequence does not function as the wild-type sequence does. For example, a protein or microRNA coding sequence may be inactivated such that the protein is not produced.

### Non-Homologous End-Joining

In one embodiment, nuclease-induced non-homologous end-joining (NHEJ) can be used to target gene-specific knockouts. Nuclease-induced NHEJ can also be used to remove (e.g., delete) sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. Two-thirds of these mutations typically alter the reading frame and, therefore, produce a non-functional protein. Additionally, mutations that maintain the reading frame, but which insert or delete a significant amount of sequence, can destroy functionality of the protein. This is locus dependent as mutations in critical functional domains are likely less tolerable than mutations in non-critical regions of the protein. The indel mutations generated by NHEJ are unpredictable in nature; however, at a given break site certain indel sequences are favored and are over represented in the population, likely due to small regions of microhomology. The lengths of deletions can vary widely; most commonly in the 1-50 bp range, but they can easily be greater than 50 bp, e.g., they can easily reach greater than about 100-200 bp. Insertions tend to be shorter and often include short duplications of the sequence immediately surrounding the break site. However, it is possible to obtain large insertions, and in these cases, the inserted sequence has often been traced to other regions of the genome or to plasmid DNA present in the cells.

Because NHEJ is a mutagenic process, it may also be used to delete small sequence motifs as long as the generation of a specific final sequence is not required. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences; however, the error-prone nature of NHEJ may still produce indel mutations at the site of repair.

Both double strand cleaving TnpB polypeptide, or an ortholog or homolog thereof, and single strand, or nickase, TnpB polypeptide, or an ortholog or homolog thereof, molecules can be used in the methods and compositions described herein to generate NHEJ-mediated indels. NHEJ-mediated indels targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp).

In an embodiment, in which a nucleic acid component molecule and TnpB polypeptide, or an ortholog or homolog thereof, generate a double strand break for the purpose of inducing NHEJ-mediated indels, an RNA component molecule may be configured to position one double-strand break in close proximity to a nucleotide of the target position. In an embodiment, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position).

In an embodiment, in which two nucleic acid component molecules complexing with TnpB polypeptide, or an ortholog or homolog thereof, e.g., TnpB polypeptide nickases induce two single strand breaks for the purpose of inducing NHEJ-mediated indels, two nucleic acid component molecules may be configured to position two single-strand breaks to provide for NHEJ repair a nucleotide of the target position.

In some examples, the systems herein may introduce one or more indels via NHEJ pathway and insert sequence from a combination template via HDR.

### Exemplary Applications

The invention provides a non-naturally occurring or engineered composition, or one or more polynucleotides encoding components of said composition, or vector or delivery systems comprising one or more polynucleotides encoding components of said composition for use in a modifying a target cell in vivo, ex vivo or in vitro and, may be conducted in a manner alters the cell such that once modified the progeny or cell line of the TnpB polypeptide modified cell retains the altered phenotype. The modified cells and progeny may be part of a multi-cellular organism such as a plant or animal with ex vivo or in vivo application of composition to desired cell types. The methods herein include a therapeutic method of treatment. The therapeutic method of treatment may comprise gene or genome editing, or gene therapy.

In one embodiment, one or more vectors described herein are used to produce a non-human transgenic animal or transgenic plant. In one embodiment, the transgenic animal is a mammal, such as a mouse, rat, or rabbit. Methods for producing transgenic animals and plants are known in the art, and generally begin with a method of cell transfection, such as described herein.

### Use of Orthogonal Catalytically Inactive TnpB Polypeptides

In particular embodiments, the TnpB polypeptide nickase is used in combination with an orthogonal catalytically inactive TnpB polypeptide to increase efficiency of said nickase (e.g., as described in Chen et al. 2017, Nature Communications 8:14958; doi: 10.1038/ncomms14958). More particularly, the orthogonal catalytically inactive TnpB polypeptide is characterized by a different TAMrecognition site than the TnpB nickase used in the AD-functionalized composition and the corresponding nucleic acid component molecule sequence is selected to bind to a target sequence proximal to that of the nickase of the functionalized TnpB polypeptide. The orthogonal catalytically inactive TnpB polypeptide as used in the context of the present invention does not form part of the functionalized composition but merely functions to increase the efficiency of said nickase and is used in combination with a standard nucleic acid component as described in the art for said TnpB polypeptide. In particular embodiments, said orthogonal catalytically inactive TnpB polypeptide is a dead TnpB polypeptide, i.e., comprising one or more mutations which abolishes the nuclease activity of said TnpB polypeptide. In particular embodiments, the catalytically inactive orthogonal TnpB polypeptide is provided with two or more nucleic acid components which are capable of hybridizing to target sequences which are proximal to the target sequence of the nickase. In particular embodiments, at least two nucleic acid components are used to target said catalytically inactive TnpB polypeptide, of which at least one nucleic acid component is capable of hybridizing to a target sequence 5″ of the target sequence of the nickase and at least one nucleic acid component is capable of hybridizing to a target sequence 3′ of the target sequence of the nickase of the functionalized composition, whereby said one or more target sequences may be on the same or the opposite DNA strand as the target sequence of the TnpB nickase. In particular embodiments, the guide sequences of the one or more @nucleic acid components of the orthogonal catalytically inactive TnpB polypeptide are selected such that the target sequences are proximal to that of the nucleic acid component for the targeting of the functionalized composition, e.g., for the targeting of the nickase. In particular embodiments, the one or more target sequences of the orthogonal catalytically inactive TnpB polypeptide are each separated from the target sequence of the nickase by more than 5 but less than 450 basepairs. Optimal distances between the target sequences of the nucleic acid component molecules for use with the orthogonal catalytically inactive TnpB polypeptide and the target sequence of the functionalized composition can be determined by the skilled person. In particular embodiments, the catalytically inactive orthogonal TnpB polypeptide has been modified to alter its TAMspecificity as described elsewhere herein. In particular embodiments, the TnpB polypeptide nickase is a nickase which, by itself has limited activity in human cells, but which, in combination with an inactive orthogonal TnpB polypeptide and one or more corresponding proximal nucleic acid component molecules ensures the required nickase activity.

### Detection Methods Such as FISH

In one aspect, the invention provides an engineered, non-naturally occurring composition comprising a catalytically inactivate TnpB polypeptide described herein, and use this system in detection methods such as fluorescence in situ hybridization (FISH). A dead TnpB polypeptide which lacks the ability to produce DNA double-strand breaks may be fused with a marker, such as fluorescent protein, such as the enhanced green fluorescent protein (eEGFP) and co-expressed with small nucleic acid component molecules to target pericentric, centric and teleomeric repeats in vivo. The dead TnpB polypeptide system can be used to visualize both repetitive sequences and individual genes in the human genome. Such new applications of labelled dead TnpB polypeptide may be important in imaging cells and studying the functional nuclear architecture, especially in cases with a small nucleus volume or complex 3-D structures. (Chen B, Gilbert L A, Cimini B A, Schnitzbauer J, Zhang W, Li G W, Park J, Blackburn E H, Weissman J S, Qi L S, Huang B. 2013. Dynamic imaging of genomic loci in living human cells by an optimized CRISPR/Cas system. Cell 155(7): 1479-91. doi: 10.1016/j.cell.2013.12.001.).

### Patient-Specific Screening Methods

A nucleic acid-targeting system that targets DNA, e.g., trinucleotide repeats can be used to screen patients or patent samples for the presence of such repeats. The repeats can be the target of the ωRNA of the TnpB system, and if there is binding thereto by the TnpB system, that binding can be detected, to thereby indicate that such a repeat is present. Thus, a TnpB system can be used to screen patients or patient samples for the presence of the repeat. The patient can then be administered suitable compound(s) to address the condition; or can be administered a TnpB system to bind to and cause insertion, deletion or mutation and alleviate the condition.

### Models of Genetic and Epigenetic Conditions

A method of the invention may be used to create a plant, an animal or cell that may be used to model and/or study genetic or epigenetic conditions of interest, such as a through a model of mutations of interest or a disease model. As used herein, “disease” refers to a disease, disorder, or indication in a subject. For example, a method of the invention may be used to create an animal or cell that comprises a modification in one or more nucleic acid sequences associated with a disease, or a plant, animal or cell in which the expression of one or more nucleic acid sequences associated with a disease are altered. Such a nucleic acid sequence may encode a disease associated protein sequence or may be a disease associated control sequence. Accordingly, it is understood that in embodiments of the invention, a plant, subject, patient, organism or cell can be a non-human subject, patient, organism or cell. Thus, the invention provides a plant, animal or cell, produced by the present methods, or a progeny thereof. The progeny may be a clone of the produced plant or animal, or may result from sexual reproduction by crossing with other individuals of the same species to introgress further desirable traits into their offspring. The cell may be in vivo or ex vivo in the cases of multicellular organisms, particularly animals or plants. In the instance where the cell is in cultured, a cell line may be established if appropriate culturing conditions are met and preferably if the cell is suitably adapted for this purpose (for instance a stem cell). Bacterial cell lines produced by the invention are also envisaged. Hence, cell lines are also envisaged.

In some methods, the disease model can be used to study the effects of mutations on the animal or cell and development and/or progression of the disease using measures commonly used in the study of the disease. Alternatively, such a disease model is useful for studying the effect of a pharmaceutically active compound on the disease.

In some methods, the disease model can be used to assess the efficacy of a potential gene therapy strategy. That is, a disease-associated gene or polynucleotide can be modified such that the disease development and/or progression is inhibited or reduced. In particular, the method comprises modifying a disease-associated gene or polynucleotide such that an altered protein is produced and, as a result, the animal or cell has an altered response. Accordingly, in some methods, a genetically modified animal may be compared with an animal predisposed to development of the disease such that the effect of the gene therapy event may be assessed.

In another embodiment, this invention provides a method of developing a biologically active agent that modulates a cell signaling event associated with a disease gene. The method comprises contacting a test compound with a cell comprising one or more vectors that drive expression of one or more of a TnpB polypeptide, and a conserved nucleotide sequence linked to a guide/spacer sequence; and detecting a change in a readout that is indicative of a reduction or an augmentation of a cell signaling event associated with, e.g., a mutation in a disease gene contained in the cell.

A cell model or animal model can be constructed in combination with the method of the invention for screening a cellular function change. Such a model may be used to study the effects of a genome sequence modified by the complex of the invention on a cellular function of interest. For example, a cellular function model may be used to study the effect of a modified genome sequence on intracellular signaling or extracellular signaling. Alternatively, a cellular function model may be used to study the effects of a modified genome sequence on sensory perception. In some such models, one or more genome sequences associated with a signaling biochemical pathway in the model are modified.

Several disease models have been specifically investigated. These include de novo autism risk genes CHD8, KATNAL2, and SCN2A; and the syndromic autism (Angelman Syndrome) gene UBE3A. These genes and resulting autism models are of course preferred, but serve to show the broad applicability of the invention across genes and corresponding models. An altered expression of one or more genome sequences associated with a signaling biochemical pathway can be determined by assaying for a difference in the mRNA levels of the corresponding genes between the test model cell and a control cell, when they are contacted with a candidate agent. Alternatively, the differential expression of the sequences associated with a signaling biochemical pathway is determined by detecting a difference in the level of the encoded polypeptide or gene product.

To assay for an agent-induced alteration in the level of mRNA transcripts or corresponding polynucleotides, nucleic acid contained in a sample is first extracted according to standard methods in the art. For instance, mRNA can be isolated using various lytic enzymes or chemical solutions according to the procedures set forth in Sambrook et al. (1989), or extracted by nucleic-acid-binding resins following the accompanying instructions provided by the manufacturers. The mRNA contained in the extracted nucleic acid sample is then detected by amplification procedures or conventional hybridization assays (e.g., Northern blot analysis) according to methods widely known in the art or based on the methods exemplified herein.

For purpose of this invention, amplification means any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. In particular, the isolated RNA can be subjected to a reverse transcription assay that is coupled with a quantitative polymerase chain reaction (RT-PCR) in order to quantify the expression level of a sequence associated with a signaling biochemical pathway.

Detection of the gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dye suitable for this application include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are well established in the art and are taught in U.S. Pat. No. 5,210,015.

In yet another aspect, conventional hybridization assays using hybridization probes that share sequence homology with sequences associated with a signaling biochemical pathway can be performed. Typically, probes are allowed to form stable complexes with the sequences associated with a signaling biochemical pathway contained within the biological sample derived from the test subject in a hybridization reaction. It will be appreciated by one of skill in the art that where antisense is used as the probe nucleic acid, the target polynucleotides provided in the sample are chosen to be complementary to sequences of the antisense nucleic acids. Conversely, where the nucleotide probe is a sense nucleic acid, the target polynucleotide is selected to be complementary to sequences of the sense nucleic acid.

Hybridization can be performed under conditions of various stringency. Suitable hybridization conditions for the practice of the present invention are such that the recognition interaction between the probe and sequences associated with a signaling biochemical pathway is both sufficiently specific and sufficiently stable. Conditions that increase the stringency of a hybridization reaction are widely known and published in the art. See, for example, (Sambrook, et al., (1989); Nonradioactive In Situ Hybridization Application Manual, Boehringer Mannheim, second edition). The hybridization assay can be formed using probes immobilized on any solid support, including but are not limited to nitrocellulose, glass, silicon, and a variety of gene arrays. A preferred hybridization assay is conducted on high-density gene chips as described in U.S. Pat. No. 5,445,934.

For a convenient detection of the probe-target complexes formed during the hybridization assay, the nucleotide probes are conjugated to a detectable label. Detectable labels suitable for use in the present invention include any composition detectable by photochemical, biochemical, spectroscopic, immunochemical, electrical, optical or chemical means. A wide variety of appropriate detectable labels are known in the art, which include fluorescent or chemiluminescent labels, radioactive isotope labels, enzymatic or other ligands. In preferred embodiments, one will likely desire to employ a fluorescent label or an enzyme tag, such as digoxigenin, β-galactosidase, urease, alkaline phosphatase or peroxidase, avidin/biotin complex.

The detection methods used to detect or quantify the hybridization intensity will typically depend upon the label selected above. For example, radiolabels may be detected using photographic film or a phosphoimager. Fluorescent markers may be detected and quantified using a photodetector to detect emitted light. Enzymatic labels are typically detected by providing the enzyme with a substrate and measuring the reaction product produced by the action of the enzyme on the substrate; and finally colorimetric labels are detected by simply visualizing the colored label.

An agent-induced change in expression of sequences associated with a signaling biochemical pathway can also be determined by examining the corresponding gene products. Determining the protein level typically involves a) contacting the protein contained in a biological sample with an agent that specifically bind to a protein associated with a signaling biochemical pathway; and (b) identifying any agent: protein complex so formed. In one aspect of this embodiment, the agent that specifically binds a protein associated with a signaling biochemical pathway is an antibody, preferably a monoclonal antibody.

The reaction is performed by contacting the agent with a sample of the proteins associated with a signaling biochemical pathway derived from the test samples under conditions that will allow a complex to form between the agent and the proteins associated with a signaling biochemical pathway. The formation of the complex can be detected directly or indirectly according to standard procedures in the art. In the direct detection method, the agents are supplied with a detectable label and unreacted agents may be removed from the complex; the amount of remaining label thereby indicating the amount of complex formed. For such method, it is preferable to select labels that remain attached to the agents even during stringent washing conditions. It is preferable that the label does not interfere with the binding reaction. In the alternative, an indirect detection procedure may use an agent that contains a label introduced either chemically or enzymatically. A desirable label generally does not interfere with binding or the stability of the resulting agent: polypeptide complex. However, the label is typically designed to be accessible to an antibody for an effective binding and hence generating a detectable signal.

A wide variety of labels suitable for detecting protein levels are known in the art. Non-limiting examples include radioisotopes, enzymes, colloidal metals, fluorescent compounds, bioluminescent compounds, and chemiluminescent compounds.

The amount of agent: polypeptide complexes formed during the binding reaction can be quantified by standard quantitative assays. As illustrated above, the formation of agent: polypeptide complex can be measured directly by the amount of label remained at the site of binding. In an alternative, the protein associated with a signaling biochemical pathway is tested for its ability to compete with a labeled analog for binding sites on the specific agent. In this competitive assay, the amount of label captured is inversely proportional to the amount of protein sequences associated with a signaling biochemical pathway present in a test sample.

A number of techniques for protein analysis based on the general principles outlined above are available in the art. They include but are not limited to radioimmunoassay, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

Antibodies that specifically recognize or bind to proteins associated with a signaling biochemical pathway are preferable for conducting the aforementioned protein analyses. Where desired, antibodies that recognize a specific type of post-translational modifications (e.g., signaling biochemical pathway inducible modifications) can be used. Post-translational modifications include but are not limited to glycosylation, lipidation, acetylation, and phosphorylation. These antibodies may be purchased from commercial vendors. For example, anti-phosphotyrosine antibodies that specifically recognize tyrosine-phosphorylated proteins are available from a number of vendors including Invitrogen and Perkin Elmer. Anti-phosphotyrosine antibodies are particularly useful in detecting proteins that are differentially phosphorylated on their tyrosine residues in response to an ER stress. Such proteins include but are not limited to eukaryotic translation initiation factor 2 alpha (eIF-2α). Alternatively, these antibodies can be generated using conventional polyclonal or monoclonal antibody technologies by immunizing a host animal or an antibody-producing cell with a target protein that exhibits the desired post-translational modification.

### Genome Wide Knock-Out Screening

The TnpB polypeptide and systems described herein can be used to perform efficient and cost effective functional genomic screens. Such screens can utilize TnpB polypeptide based genome wide libraries. Such screens and libraries can provide for determining the function of genes, cellular pathways genes are involved in, and how any alteration in gene expression can result in a particular biological process. An advantage of the present invention is that the composition avoids off-target binding and its resulting side effects. This is achieved using systems arranged to have a high degree of sequence specificity for the target DNA. In preferred embodiments of the invention, the TnpB polypeptide complexes are TnpB polypeptide complexes.

In embodiments of the invention, a genome wide library may comprise a plurality of TnpB polypeptide nucleic acid component molecules, as described herein, comprising guide/spacer sequences that are capable of targeting a plurality of target sequences in a plurality of genomic loci in a population of eukaryotic cells. The population of cells may be a population of embryonic stem (ES) cells. The target sequence in the genomic locus may be a non-coding sequence. The non-coding sequence may be an intron, regulatory sequence, splice site, 3′ UTR, 5′ UTR, or polyadenylation signal. Gene function of one or more gene products may be altered by said targeting. The targeting may result in a knockout of gene function. The targeting of a gene product may comprise more than one nucleic acid component molecule. A gene product may be targeted by 2, 3, 4, 5, 6, 7, 8, 9, or 10 nucleic acid component molecules, preferably 3 to 4 per gene. Off-target modifications may be minimized by exploiting the staggered double strand breaks generated by TnpB polypeptide complexes or by utilizing methods analogous to those used in composition (See, e.g., DNA targeting specificity of RNA-guided Cas nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi: 10.1038/nbt.2647(2013)), incorporated herein by reference. The targeting may be of about 100 or more sequences. The targeting may be of about 1000 or more sequences. The targeting may be of about 20,000 or more sequences. The targeting may be of the entire genome. The targeting may be of a panel of target sequences focused on a relevant or desirable pathway. The pathway may be an immune pathway. The pathway may be a cell division pathway.

One aspect of the invention comprehends a genome wide library that may comprise a plurality of nucleic acid component molecules that may comprise guide/spacer sequences that are capable of targeting a plurality of target sequences in a plurality of genomic loci, wherein said targeting results in a knockout of gene function. This library may potentially comprise nucleic acid component molecules that target each and every gene in the genome of an organism.

In one embodiment of the invention, the organism or subject is a eukaryote (including mammal including human) or a non-human eukaryote or a non-human animal or a non-human mammal. In one embodiment, the organism or subject is a non-human animal, and may be an arthropod, for example, an insect, or may be a nematode. In some methods of the invention the organism or subject is a plant. In some methods of the invention, the organism or subject is a mammal or a non-human mammal. A non-human mammal may be, for example, a rodent (preferably a mouse or a rat), an ungulate, or a primate. In some methods of the invention, the organism or subject is algae, including microalgae, or is a fungus.

The knockout of gene function may comprise introducing into each cell in the population of cells a vector system of one or more vectors comprising an engineered, non-naturally occurring composition herein. The nucleic acid component molecule sequence may target a unique gene in each cell, wherein the TnpB polypeptide is operably linked to a regulatory element, wherein when transcribed, the nucleic acid component molecule comprising the spacer sequence directs sequence-specific binding of the TnpB polypeptide to a target sequence in the genomic loci of the unique gene, inducing cleavage of the genomic loci by the TnpB polypeptide, and confirming different knockout mutations in a plurality of unique genes in each cell of the population of cells thereby generating a gene knockout cell library. The invention comprehends that the population of cells is a population of eukaryotic cells, and in a preferred embodiment, the population of cells is a population of embryonic stem (ES) cells.

The one or more vectors may be plasmid vectors. The vector may be a single vector comprising a TnpB polypeptide, a nucleic acid component, and optionally, a selection marker into target cells. Not being bound by a theory, the ability to simultaneously deliver a TnpB polypeptide and nucleic acid component through a single vector enables application to any cell type of interest, without the need to first generate cell lines that express the TnpB polypeptide. The regulatory element may be an inducible promoter. The inducible promoter may be a doxycycline inducible promoter. In some methods of the invention the expression of the nucleic acid component molecule sequence is under the control of the T7 promoter and is driven by the expression of T7 polymerase. The confirming of different knockout mutations may be by whole exome sequencing. The knockout mutation may be achieved in 100 or more unique genes. The knockout mutation may be achieved in 1000 or more unique genes. The knockout mutation may be achieved in 20,000 or more unique genes. The knockout mutation may be achieved in the entire genome. The knockout of gene function may be achieved in a plurality of unique genes which function in a particular physiological pathway or condition. The pathway or condition may be an immune pathway or condition. The pathway or condition may be a cell division pathway or condition.

### Functional Alteration and Screening

In another aspect, the present invention provides for a method of functional evaluation and screening of genes. The use of the compositions to precisely deliver functional domains, to activate or repress genes or to alter epigenetic state by precisely altering the methylation site on a specific locus of interest, can be with one or more nucleic acid component molecules applied to a single cell or population of cells or with a library applied to genome in a pool of cells ex vivo or in vivo comprising the administration or expression of a library comprising a plurality of nucleic acid components (comprising spacer molecules) and wherein the screening further comprises use of a TnpB polypeptide, wherein the complex comprising the TnpB polypeptide is modified to comprise a heterologous functional domain. In an aspect, the invention provides a method for screening a genome comprising the administration to a host or expression in a host in vivo of a library. In an aspect, the invention provides a method as herein discussed further comprising an activator administered to the host or expressed in the host. In an aspect, the invention provides a method as herein discussed wherein the activator is attached to a TnpB polypeptide. In an aspect, the invention provides a method as herein discussed wherein the activator is attached to the N terminus or the C terminus of the TnpB polypeptide. In an aspect, the invention provides a method as herein discussed wherein the activator is attached to a nucleic acid component loop. In an aspect, the invention provides a method as herein discussed further comprising a repressor administered to the host or expressed in the host. In an aspect, the invention provides a method as herein discussed, wherein the screening comprises affecting and detecting gene activation, gene inhibition, or cleavage in the locus.

It is also preferred to target endogenous (regulatory) control elements (such as enhancers and silencers) e.g., in addition to a promoter or promoter-proximal elements. Thus, the invention can also be used to target endogenous control elements (including enhancers and silencers) in addition to targeting of the promoter. These control elements can be located upstream and downstream of the transcriptional start site (TSS), starting from 200 bp from the TSS to 100 kb away. Targeting of known control elements can be used to activate or repress the gene of interest. In some cases, a single control element can influence the transcription of multiple target genes. Targeting of a single control element could therefore be used to control the transcription of multiple genes simultaneously.

Targeting of putative control elements on the other hand (e.g., by tiling the region of the putative control element as well as 200 bp up to 100 kB around the element) can be used as a means to verify such elements (by measuring the transcription of the gene of interest) or to detect novel control elements (e.g., by tiling 100 kb upstream and downstream of the TSS of the gene of interest). In addition, targeting of putative control elements can be useful in the context of understanding genetic causes of disease. Many mutations and common SNP variants associated with disease phenotypes are located outside coding regions. Targeting of such regions with either the activation or repression systems described herein can be followed by readout of transcription of either a) a set of putative targets (e.g., a set of genes located in closest proximity to the control element) or b) whole-transcriptome readout by e.g., RNAseq or microarray. This would allow for the identification of likely candidate genes involved in the disease phenotype. Such candidate genes could be useful as novel drug targets.

Histone acetyltransferase (HAT) inhibitors are mentioned herein. However, an alternative embodiment is for the one or more functional domains to comprise an acetyltransferase, preferably a histone acetyltransferase. These are useful in the field of epigenomics, for example in methods of interrogating the epigenome. Methods of interrogating the epigenome may include, for example, targeting epigenomic sequences. Targeting epigenomic sequences may include the nucleic acid component molecule being directed to an epigenomic target sequence. Epigenomic target sequence may include a promoter, silencer or an enhancer sequence.

### Saturating Mutagenesis

The compositions herein can be used to perform saturating or deep scanning mutagenesis of genomic loci in conjunction with a cellular phenotype—for instance, for determining critical minimal features and discrete vulnerabilities of functional elements required for gene expression, drug resistance, and reversal of disease. By saturating or deep scanning mutagenesis is meant that every or essentially every DNA base is cut within the genomic loci. A library of Cas1 effector protein nucleic acid component molecules may be introduced into a population of cells. The library may be introduced, such that each cell receives a single nucleic acid component. In the case where the library is introduced by transduction of a viral vector, as described herein, a low multiplicity of infection (MOI) is used. The library may include nucleic acid components targeting every sequence upstream of a (targeted adjacent motif) (TAM) sequence in a genomic locus. The library may include at least 100 non-overlapping genomic sequences upstream of a TAM sequence for every 1000 base pairs within the genomic locus. The library may include nucleic acid components targeting sequences upstream of at least one different TAM sequence. The composition may include more than one TnpB polypeptide. Any TnpB polypeptide protein as described herein, including orthologues or engineered TnpB polypeptides. The frequency of off target sites for a nucleic acid component may be less than 500. Off target scores may be generated to select nucleic acid components with the lowest off target sites. Any phenotype determined to be associated with cutting at a nucleic acid component target site may be confirmed by using nucleic acid components targeting the same site in a single experiment. Validation of a target site may also be performed by using a modified TnpB polypeptide, as described herein, and two nucleic acid components targeting the genomic site of interest. Not being bound by a theory, a target site is a true hit if the change in phenotype is observed in validation experiments.

The genomic loci may include at least one continuous genomic region. The at least one continuous genomic region may comprise up to the entire genome. The at least one continuous genomic region may comprise a functional element of the genome. The functional element may be within a non-coding region, coding gene, intronic region, promoter, or enhancer. The at least one continuous genomic region may comprise at least 1 kb, preferably at least 50 kb of genomic DNA. The at least one continuous genomic region may comprise a transcription factor binding site. The at least one continuous genomic region may comprise a region of DNase I hypersensitivity. The at least one continuous genomic region may comprise a transcription enhancer or repressor element. The at least one continuous genomic region may comprise a site enriched for an epigenetic signature. The at least one continuous genomic DNA region may comprise an epigenetic insulator. The at least one continuous genomic region may comprise two or more continuous genomic regions that physically interact. Genomic regions that interact may be determined by ‘4C technology’. 4C technology allows the screening of the entire genome in an unbiased manner for DNA segments that physically interact with a DNA fragment of choice, as is described in Zhao et al. ((2006) Nat Genet 38, 1341-7) and in U.S. Pat. No. 8,642,295, both incorporated herein by reference in its entirety. The epigenetic signature may be histone acetylation, histone methylation, histone ubiquitination, histone phosphorylation, DNA methylation, or a lack thereof.

The compositions for saturating or deep scanning mutagenesis can be used in a population of cells. The compositions can be used in eukaryotic cells, including but not limited to mammalian and plant cells. The population of cells may be prokaryotic cells. The population of eukaryotic cells may be a population of embryonic stem (ES) cells, neuronal cells, epithelial cells, immune cells, endocrine cells, muscle cells, erythrocytes, lymphocytes, plant cells, or yeast cells.

In one aspect, the present invention provides for a method of screening for functional elements associated with a change in a phenotype. The library may be introduced into a population of cells that are adapted to contain a TnpB polypeptide. The cells may be sorted into at least two groups based on the phenotype. The phenotype may be expression of a gene, cell growth, or cell viability. The relative representation of the nucleic acid component molecules present in each group are determined, whereby genomic sites associated with the change in phenotype are determined by the representation of nucleic acid component molecules present in each group. The change in phenotype may be a change in expression of a gene of interest. The gene of interest may be upregulated, downregulated, or knocked out. The cells may be sorted into a high expression group and a low expression group. The population of cells may include a reporter construct that is used to determine the phenotype. The reporter construct may include a detectable marker. Cells may be sorted by use of the detectable marker.

In another aspect, the present invention provides for a method of screening for genomic sites associated with resistance to a chemical compound. The chemical compound may be a drug or pesticide. The library may be introduced into a population of cells that are adapted to contain a TnpB polypeptide, wherein each cell of the population contains no more than one nucleic acid component molecule; the population of cells are treated with the chemical compound; and the representation of nucleic acid component molecules are determined after treatment with the chemical compound at a later time point as compared to an early time point, whereby genomic sites associated with resistance to the chemical compound are determined by enrichment of nucleic acid components. Representation of nucleic acid components may be determined by deep sequencing methods.

Useful in the practice of the instant invention utilizing compositions are methods used in compositions and reference is made to the article entitled BCL11A enhancer dissection by Cas-mediated in situ saturating mutagenesis. Canver, M. C., Smith, E. C., Sher, F., Pinello, L., Sanjana, N. E., Shalem, O., Chen, D. D., Schupp, P. G., Vinjamur, D. S., Garcia, S. P., Luc, S., Kurita, R., Nakamura, Y., Fujiwara, Y., Maeda, T., Yuan, G., Zhang, F., Orkin, S. H., & Bauer, D. E. DOI: 10.1038/nature15521, published online Sep. 16, 2015, the article is herein incorporated by reference and discussed briefly below: Canver et al. involves novel pooled guide RNA libraries to perform in situ saturating mutagenesis of the human and mouse BCL11A erythroid enhancers previously identified as an enhancer associated with fetal hemoglobin (HbF) level and whose mouse ortholog is necessary for erythroid BCL11A expression. This approach revealed critical minimal features and discrete vulnerabilities of these enhancers. Through editing of primary human progenitors and mouse transgenesis, the authors validated the BCL11A erythroid enhancer as a target for HbF reinduction. The authors generated a detailed enhancer map that informs therapeutic genome editing.

### Modification of a Cell or Organism

The present disclosure further provides cells comprising one or more components of the systems herein, e.g., the TnpB polypeptide and/or nucleic acid component(s). Also provided include cells modified by the systems and methods herein, and cell cultures, tissues, organs, organism comprising such cells or progeny thereof. The invention In one embodiment comprehends a method of modifying an cell or organism. The cell may be a prokaryotic cell or a eukaryotic cell. The cell may be a mammalian cell. The mammalian cell many be a non-human primate, bovine, porcine, rodent or mouse cell. The cell may be a non-mammalian eukaryotic cell such as poultry, fish or shrimp. The cell may also be a plant cell. The plant cell may be of a crop plant such as cassava, corn, sorghum, wheat, or rice. The plant cell may also be of an algae, tree or vegetable. The modification introduced to the cell by the present invention may be such that the cell and progeny of the cell are altered for improved production of biologic products such as an antibody, starch, alcohol or other desired cellular output. The modification introduced to the cell by the present invention may be such that the cell and progeny of the cell include an alteration that changes the biologic product produced.

### Therapeutic Uses and Methods of Treatment

Also provided herein are methods of diagnosing, prognosing, treating, and/or preventing a disease, state, or condition in or of a subject. Generally, the methods of diagnosing, prognosing, treating, and/or preventing a disease, state, or condition in or of a subject can include modifying a polynucleotide in a subject or cell thereof using a composition, system, or component thereof described herein and/or include detecting a diseased or healthy polynucleotide in a subject or cell thereof using a composition, system, or component thereof described herein. In one embodiment, the method of treatment or prevention can include using a composition, system, or component thereof to modify a polynucleotide of an infectious organism (e.g., bacterial or virus) within a subject or cell thereof. In one embodiment, the method of treatment or prevention can include using a composition, system, or component thereof to modify a polynucleotide of an infectious organism or symbiotic organism within a subject. The composition, system, and components thereof can be used to develop models of diseases, states, or conditions. The composition, system, and components thereof can be used to detect a disease state or correction thereof, such as by a method of treatment or prevention described herein. The composition, system, and components thereof can be used to screen and select cells that can be used, for example, as treatments or preventions described herein. The composition, system, and components thereof can be used to develop biologically active agents that can be used to modify one or more biologic functions or activities in a subject or a cell thereof.

In general, the method can include delivering a composition, system, and/or component thereof to a subject or cell thereof, or to an infectious or symbiotic organism by a suitable delivery technique and/or composition. Once administered the components can operate as described elsewhere herein to elicit a nucleic acid modification event. In some aspects, the nucleic acid modification event can occur at the genomic, epigenomic, and/or transcriptomic level. DNA and/or RNA cleavage, gene activation, and/or gene deactivation can occur. Additional features, uses, and advantages are described in greater detail below. On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different functional domains to elicit one or more genomic locus events. In addition to treating and/or preventing a disease in a subject, the compositions may be applied in a wide variety of methods for screening in libraries in cells and functional modeling in vivo (e.g., gene activation of lincRNA and identification of function; gain-of-function modeling; loss-of-function modeling; the use the compositions of the invention to establish cell lines and transgenic animals for optimization and screening purposes).

The composition, system, and components thereof described elsewhere herein can be used to treat and/or prevent a disease, such as a genetic and/or epigenetic disease, in a subject. The composition, system, and components thereof described elsewhere herein can be used to treat and/or prevent genetic infectious diseases in a subject, such as bacterial infections, viral infections, fungal infections, parasite infections, and combinations thereof. The composition, system, and components thereof described elsewhere herein can be used to modify the composition or profile of a microbiome in a subject, which can in turn modify the health status of the subject. The composition, system, described herein can be used to modify cells ex vivo, which can then be administered to the subject whereby the modified cells can treat or prevent a disease or symptom thereof. This is also referred to in some contexts as adoptive therapy. The composition, system, described herein can be used to treat mitochondrial diseases, where the mitochondrial disease etiology involves a mutation in the mitochondrial DNA.

Also provided is a method of treating a subject, e.g., a subject in need thereof, comprising inducing gene editing by transforming the subject with the polynucleotide encoding one or more components of the composition, system, or complex or any of polynucleotides or vectors described herein and administering them to the subject. A suitable repair template may also be provided, for example delivered by a vector comprising said repair template. The repair template may be a recombination template herein. Also provided is a method of treating a subject, e.g., a subject in need thereof, comprising inducing transcriptional activation or repression of multiple target gene loci by transforming the subject with the polynucleotides or vectors described herein, wherein said polynucleotide or vector encodes or comprises one or more components of composition, system, complex or component thereof comprising multiple TnpB polypeptides. Where any treatment is occurring ex vivo, for example in a cell culture, then it will be appreciated that the term ‘subject’ may be replaced by the phrase “cell or cell culture.”

Also provided is a method of treating a subject, e.g., a subject in need thereof, comprising inducing gene editing by transforming the subject with the TnpB polypeptide(s), advantageously encoding and expressing in vivo the remaining portions of the composition, system, (e.g., RNA). A suitable repair template may also be provided, for example delivered by a vector comprising said repair template. Also provided is a method of treating a subject, e.g., a subject in need thereof, comprising inducing transcriptional activation or repression by transforming the subject with the TnpB polypeptide(s) advantageously encoding and expressing in vivo the remaining portions of the composition, system, (e.g., nucleic acid component molecule); advantageously in one embodiment, the TnpB polypeptide is a catalytically inactive TnpB polypeptide and includes one or more associated functional domains. Where any treatment is occurring ex vivo, for example in a cell culture, then it will be appreciated that the term ‘subject’ may be replaced by the phrase “cell or cell culture.”

One or more components of the composition and system described herein can be included in a composition, such as a pharmaceutical composition, and administered to a host individually or collectively. Alternatively, these components may be provided in a single composition for administration to a host. Administration to a host may be performed via viral vectors known to the skilled person or described herein for delivery to a host (e.g., lentiviral vector, adenoviral vector, AAV vector). As explained herein, use of different selection markers (e.g., for lentiviral nucleic acid component selection) and concentration of nucleic acid component (e.g., dependent on whether multiple nucleic acid components are used) may be advantageous for eliciting an improved effect.

Thus, also described herein are methods of inducing one or more polynucleotide modifications in a eukaryotic or prokaryotic cell or component thereof (e.g., a mitochondria) of a subject, infectious organism, and/or organism of the microbiome of the subject. The modification can include the introduction, deletion, or substitution of one or more nucleotides at a target sequence of a polynucleotide of one or more cell(s). The modification can occur in vitro, ex vivo, in situ, or in vivo.

In one embodiment, the method of treating or inhibiting a condition or a disease caused by one or more mutations in a genomic locus in a eukaryotic organism or a non-human organism can include manipulation of a target sequence within a coding, non-coding or regulatory element of said genomic locus in a target sequence in a subject or a non-human subject in need thereof comprising modifying the subject or a non-human subject by manipulation of the target sequence and wherein the condition or disease is susceptible to treatment or inhibition by manipulation of the target sequence including providing treatment comprising delivering a composition comprising the particle delivery system or the delivery system or the virus particle of any one of the above embodiment or the cell of any one of the above embodiment.

Also provided herein is the use of the particle delivery system or the delivery system or the virus particle of any one of the above embodiments or the cell of any one of the above embodiments in ex vivo or in vivo gene or genome editing; or for use in in vitro, ex vivo or in vivo gene therapy. Also provided herein are particle delivery systems, non-viral delivery systems, and/or the virus particle of any one of the above embodiments or the cell of any one of the above embodiments used in the manufacture of a medicament for in vitro, ex vivo or in vivo gene or genome editing or for use in in vitro, ex vivo or in vivo gene therapy or for use in a method of modifying an organism or a non-human organism by manipulation of a target sequence in a genomic locus associated with a disease or in a method of treating or inhibiting a condition or disease caused by one or more mutations in a genomic locus in a eukaryotic organism or a non-human organism.

In one embodiment, polynucleotide modification can include the introduction, deletion, or substitution of 1-75 nucleotides at each target sequence of said polynucleotide of said cell(s). The modification can include the introduction, deletion, or substitution of at least 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence. The modification can include the introduction, deletion, or substitution of at least 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of at least 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of at least 20, 21, 22, 23, 24, 25, 26, 27, 28, 29, 30, 35, 40, 45, 50, or 75 nucleotides at each target sequence of said cell(s). The modification can include the introduction, deletion, or substitution of at least 40, 45, 50, 75, 100, 200, 300, 400 or 500 nucleotides at each target sequence of said cell(s). The modification can include the introduction, deletion, or substitution of at least 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, or 9900 to 10000 nucleotides at each target sequence of said cell(s).

In one embodiment, the modifications can include the introduction, deletion, or substitution of nucleotides at each target sequence of said cell(s) via nucleic acid components (e.g., nucleic acid component molecule(s) RNA(s) or nucleic acid component(s)), such as those mediated by a composition, system, or a component thereof described elsewhere herein. In one embodiment, the modifications can include the introduction, deletion, or substitution of nucleotides at a target or random sequence of said cell(s) via a composition, system, or technique.

In one embodiment, the composition, system, or component thereof can promote Non-Homologous End-Joining (NHEJ). In one embodiment, modification of a polynucleotide by a composition, system, or a component thereof, such as a diseased polynucleotide, can include NHEJ. In one embodiment, promotion of this repair pathway by the composition, system, or a component thereof can be used to target gene or polynucleotide specific knock-outs and/or knock-ins. In one embodiment, promotion of this repair pathway by the composition, system, or a component thereof can be used to generate NHEJ-mediated indels. Nuclease-induced NHEJ can also be used to remove (e.g., delete) sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. The indel can range in size from 1-50 or more base pairs. In one embodiment thee indel can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 base pairs or more. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences.

In one embodiment, composition, system, mediated NHEJ can be used in the method to delete small sequence motifs. In one embodiment, composition, system, mediated NHEJ can be used in the method to generate NHEJ-mediate indels that can be targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp). In an embodiment, in which a nucleic acid component and TnpB polypeptide generate a double strand break for the purpose of inducing NHEJ-mediated indels, a nucleic acid component may be configured to position one double-strand break in close proximity to a nucleotide of the target position. In an embodiment, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position). In an embodiment, in which two component RNAs complexing with one or more nickases induce two single strand breaks for the purpose of inducing NHEJ-mediated indels, two component RNAs may be configured to position two single-strand breaks to provide for NHEJ repair a nucleotide of the target position.

For minimization of toxicity and off-target effect, it may be important to control the concentration of TnpB polypeptide mRNA and component RNA delivered. Optimal concentrations of TnpB polypeptide mRNA and component RNA can be determined by testing different concentrations in a cellular or non-human eukaryote animal model and using deep sequencing the analyze the extent of modification at potential off-target genomic loci. Alternatively, to minimize the level of toxicity and off-target effect, nickase mRNA (for example a mutated TnpB) can be delivered with a pair of nucleic acid components targeting a site of interest.

Typically, in the context of an endogenous TnpB polypeptide, formation of a TnpB polypeptide or complex (comprising a polynucleotide component sequence hybridized to a target sequence and complexed with one or more TnpB polypeptides) results in cleavage, nicking, and/or another modification of one or both strands in or near (e.g., within 1, 2, 3, 4, 5, 6, 7, 8, 9, 10, 20, 50, or more base pairs from) the target sequence.

In one embodiment, a method of modifying a target polynucleotide in a cell to treat or prevent a disease can include allowing a composition, system, or component thereof to bind to the target polynucleotide, e.g., to effect cleavage, nicking, or other modification as the composition, system, is capable of said target polynucleotide, thereby modifying the target polynucleotide, wherein the composition, system, or component thereof, complex with a nucleic acid component molecule sequence, and hybridize said nucleic acid component molecule sequence to a target sequence within the target polynucleotide, wherein said nucleic acid component molecule sequence is optionally linked to a nucleic acid component scaffold sequence. In some of these embodiments, the composition, system, or component thereof can be or include a TnpB polypeptide complexed with a nucleic acid component molecule sequence. In one embodiment, modification can include cleaving or nicking one or two strands at the location of the target sequence by one or more components of the composition, system, or component thereof.

The cleavage, nicking, or other modification capable of being performed by the composition, system, can modify transcription of a target polynucleotide. In one embodiment, modification of transcription can include decreasing transcription of a target polynucleotide. In one embodiment, modification can include increasing transcription of a target polynucleotide. In one embodiment, the method includes repairing said cleaved target polynucleotide by homologous recombination with a recombination template polynucleotide, wherein said repair results in a modification such as, but not limited to, an insertion, deletion, or substitution of one or more nucleotides of said target polynucleotide. In one embodiment, said modification results in one or more amino acid changes in a protein expressed from a gene comprising the target sequence. In one embodiment, the modification imparted by the composition, system, or component thereof provides a transcript and/or protein that can correct a disease or a symptom thereof, including but not limited to, any of those described in greater detail elsewhere herein.

In one embodiment, the method of treating or preventing a disease can include delivering one or more vectors or vector systems to a cell, such as a eukaryotic or prokaryotic cell, wherein one or more vectors or vector systems include the composition, system, or component thereof. In one embodiment, the vector(s) or vector system(s) can be a viral vector or vector system, such as an AAV or lentiviral vector system, which are described in greater detail elsewhere herein. In one embodiment, the method of treating or preventing a disease can include delivering one or more viral particles, such as an AAV or lentiviral particle, containing the composition, system, or component thereof. In one embodiment, the viral particle has a tissue specific tropism. In one embodiment, the viral particle has a liver, muscle, eye, heart, pancreas, kidney, neuron, epithelial cell, endothelial cell, astrocyte, glial cell, immune cell, or red blood cell specific tropism.

It will be understood that the composition and system, according to the invention as described herein, such as the composition and system, for use in the methods according to the invention as described herein, may be suitably used for any type of application known for composition, system, preferably in eukaryotes. In certain aspects, the application is therapeutic, preferably therapeutic in a eukaryote organism, such as including but not limited to animals (including human), plants, algae, fungi (including yeasts), etc. Alternatively, or in addition, in certain aspects, the application may involve accomplishing or inducing one or more particular traits or characteristics, such as genotypic and/or phenotypic traits or characteristics, as also described elsewhere herein.

### Treating Diseases of the Circulatory System

In one embodiment, the composition, system, and/or component thereof described herein can be used to treat and/or prevent a circulatory system disease. Exemplary disease is provided, for example, in Tables 2 and 3. In one embodiment the plasma exosomes of Wahlgren et al. (Nucleic Acids Research, 2012, Vol. 40, No. 17 e130) can be used to deliver the composition, system, and/or component thereof described herein to the blood. In one embodiment, the circulatory system disease can be treated by using a lentivirus to deliver the composition, system, described herein to modify hematopoietic stem cells (HSCs) in vivo or ex vivo (see e.g., Drakopoulou, “Review Article, The Ongoing Challenge of Hematopoietic Stem Cell-Based Gene Therapy for β-Thalassemia,” Stem Cells International, Volume 2011, Article ID 987980, 10 pages, doi: 10.4061/2011/987980, which can be adapted for use with the composition, system, herein in view of the description herein). In one embodiment, the circulatory system disorder can be treated by correcting HSCs as to the disease using a composition, system, herein or a component thereof, wherein the composition, system, optionally includes a suitable HDR repair template (see e.g., Cavazzana, “Outcomes of Gene Therapy for β-Thalassemia Major via Transplantation of Autologous Hematopoietic Stem Cells Transduced Ex Vivo with a Lentiviral βA-T87Q-Globin Vector.”; Cavazzana-Calvo, “Transfusion independence and HMGA2 activation after gene therapy of human β-thalassaemia”, Nature 467, 318-322(16 Sep. 2010) doi: 10.1038/nature09328; Nienhuis, “Development of Gene Therapy for Thalassemia, Cold Spring Harbor Perspectives in Medicine, doi: 10.1101/cshperspect.a011833(2012), LentiGlobin BB305, a lentiviral vector containing an engineered β-globin gene (BA-T87Q); and Xie et al., “Seamless gene correction of β-thalassaemia mutations in patient-specific iPSCs using CRISPR/Cas9 and piggyback” Genome Research gr. 173427.114(2014) www.genome.org/cgi/doi/10.1101/gr.173427.114 (Cold Spring Harbor Laboratory Press; Watts, “Hematopoietic Stem Cell Expansion and Gene Therapy” Cytotherapy 13(10): 1164-1171. doi: 10.3109/14653249.2011.620748(2011), which can be adapted for use with the composition, system, herein in view of the description herein). In one embodiment, iPSCs can be modified using a composition, system, described herein to correct a disease polynucleotide associated with a circulatory disease. In this regard, the teachings of Xu et al. (Sci Rep. 2015 Jul. 9; 5:12065. doi: 10.1038/srep12065) and Song et al. (Stem Cells Dev. 2015 May 1; 24(9): 1053-65. doi: 10.1089/scd.2014.0347. Epub 2015 Feb. 5) with respect to modifying iPSCs can be adapted for use in view of the description herein with the composition, system, described herein.

The term “Hematopoietic Stem Cell” or “HSC” refers broadly those cells considered to be an HSC, e.g., blood cells that give rise to all the other blood cells and are derived from mesoderm; located in the red bone marrow, which is contained in the core of most bones. HSCs of the invention include cells having a phenotype of hematopoietic stem cells, identified by small size, lack of lineage (lin) markers, and markers that belong to the cluster of differentiation series, like: CD34, CD38, CD90, CD133, CD105, CD45, and also c-kit,—the receptor for stem cell factor. Hematopoietic stem cells are negative for the markers that are used for detection of lineage commitment, and are, thus, called Lin−; and, during their purification by FACS, a number of up to 14 different mature blood-lineage markers, e.g., CD13 & CD33 for myeloid, CD71 for erythroid, CD19 for B cells, CD61 for megakaryocytic, etc. for humans; and, B220 (murine CD45) for B cells, Mac-1 (CD11b/CD18) for monocytes, Gr-1 for Granulocytes, Ter119 for erythroid cells, Il7Ra, CD3, CD4, CD5, CD8 for T cells, etc. Mouse HSC markers: CD34lo/−, SCA-1+, Thy 1.1+/lo, CD38+, C-kit+, lin−, and Human HSC markers: CD34+, CD59+, Thy1/CD90+, CD38lo/−, C-kit/CD117+, and lin−. HSCs are identified by markers. Hence in embodiments discussed herein, the HSCs can be CD34+ cells. HSCs can also be hematopoietic stem cells that are CD34−/CD38−. Stem cells that may lack c-kit on the cell surface that are considered in the art as HSCs are within the ambit of the invention, as well as CD133+ cells likewise considered HSCs in the art.

In one embodiment, the treatment or prevention for treating a circulatory system or blood disease can include modifying a human cord blood cell with any modification described herein. In one embodiment, the treatment or prevention for treating a circulatory system or blood disease can include modifying a granulocyte colony-stimulating factor-mobilized peripheral blood cell (mPB) with any modification described herein. In one embodiment, the human cord blood cell or mPB can be CD34+. In one embodiment, the cord blood cell(s) or mPB cell(s) modified can be autologous. In one embodiment, the cord blood cell(s) or mPB cell(s) can be allogenic. In addition to the modification of the disease gene(s), allogenic cells can be further modified using the composition, system, described herein to reduce the immunogenicity of the cells when delivered to the recipient. Such techniques are described elsewhere herein and e.g., Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20(2010) 857-862, which can be adapted for use with the composition, system, herein. The modified cord blood cell(s) or mPB cell(s) can be optionally expanded in vitro. The modified cord blood cell(s) or mPB cell(s) can be derived to a subject in need thereof using any suitable delivery technique.

The compositions may be engineered to target genetic locus or loci in HSCs. In one embodiment, the TnpB polypeptide(s) can be codon-optimized for a eukaryotic cell and especially a mammalian cell, e.g., a human cell, for instance, HSC, or iPSC and nucleic acid component targeting a locus or loci in HSC, such as circulatory disease, can be prepared. These may be delivered via particles. The particles may be formed by the TnpB polypeptide, and the nucleic acid component being admixed. The nucleic acid component and TnpB polypeptide mixture can be, for example, admixed with a mixture comprising or consisting essentially of or consisting of surfactant, phospholipid, biodegradable polymer, lipoprotein and alcohol, whereby particles containing the nucleic acid component and TnpB polypeptide may be formed. The invention comprehends so making particles and particles from such a method as well as uses thereof. Particles suitable delivery of the composition in the context of blood or circulatory system or HSC delivery to the blood or circulatory system are described in greater detail elsewhere herein.

In one embodiment, after ex vivo modification the HSCs or iPCS can be expanded prior to administration to the subject. Expansion of HSCs can be via any suitable method such as that described by, Lee, “Improved ex vivo expansion of adult hematopoietic stem cells by overcoming CUL4-mediated degradation of HOXB4.” Blood. 2013 May 16; 121(20): 4082-9. doi: 10.1182/blood-2012-09-455204. Epub 2013 Mar. 21.

In one embodiment, the HSCs or iPSCs modified can be autologous. In one embodiment, the HSCs or iPSCs can be allogenic. In addition to the modification of the disease gene(s), allogenic cells can be further modified using the composition, system, described herein to reduce the immunogenicity of the cells when delivered to the recipient. Such techniques are described elsewhere herein and e.g., Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20(2010) 857-862, which can be adapted for use with the composition, system, herein.

### Treating Neurological Diseases

In one embodiment, the compositions, systems, described herein can be used to treat diseases of the brain and CNS. Delivery options for the brain include encapsulation of TnpB polypeptide and nucleic acid component molecule in the form of either DNA or RNA into liposomes and conjugating to molecular Trojan horses for trans-blood brain barrier (BBB) delivery. Molecular Trojan horses have been shown to be effective for delivery of B-gal expression vectors into the brain of non-human primates. The same approach can be used to delivery vectors containing TnpB polypeptide and nucleic acid component molecule. For instance, Xia C F and Boado R J, Pardridge W M (“Antibody-mediated targeting of siRNA via the human insulin receptor using avidin-biotin technology.” Mol Pharm. 2009 May-June; 6(3): 747-51. doi: 10.1021/mp800194) describes how delivery of short interfering RNA (siRNA) to cells in culture, and in vivo, is possible with combined use of a receptor-specific monoclonal antibody (mAb) and avidin-biotin technology. The authors also report that because the bond between the targeting mAb and the siRNA is stable with avidin-biotin technology, and RNAi effects at distant sites such as brain are observed in vivo following an intravenous administration of the targeted siRNA, the teachings of which can be adapted for use with the compositions, systems, herein. In other embodiments, an artificial virus can be generated for CNS and/or brain delivery. See e.g., Zhang et al. (Mol Ther. 2003 January; 7(1): 11-8.)), the teachings of which can be adapted for use with the compositions, systems, herein.

### Treating Hearing Diseases

In one embodiment the composition and system described herein can be used to treat a hearing disease or hearing loss in one or both ears. Deafness is often caused by lost or damaged hair cells that cannot relay signals to auditory neurons. In such cases, cochlear implants may be used to respond to sound and transmit electrical signals to the nerve cells. But these neurons often degenerate and retract from the cochlea as fewer growth factors are released by impaired hair cells.

In one embodiment, the composition, system, or modified cells can be delivered to one or both ears for treating or preventing hearing disease or loss by any suitable method or technique. Suitable methods and techniques include, but are not limited to, those set forth in US Patent Publication No. 20120328580 describes injection of a pharmaceutical composition into the ear (e.g., auricular administration), such as into the luminae of the cochlea (e.g., the Scala media, Sc vestibulae, and Sc tympani), e.g., using a syringe, e.g., a single-dose syringe. For example, one or more of the compounds described herein can be administered by intratympanic injection (e.g., into the middle ear), and/or injections into the outer, middle, and/or inner ear; administration in situ, via a catheter or pump (see e.g., McKenna et al., (U.S. Patent Publication No. 2006/0030837) and Jacobsen et al., (U.S. Pat. No. 7,206,639); administration in combination with a mechanical device such as a cochlear implant or a hearing aid, which is worn in the outer ear (see e.g., U.S. Patent Publication No. 2007/0093878, which provides an exemplary cochlear implant suitable for delivery of the compositions, systems, described herein to the ear). Such methods are routinely used in the art, for example, for the administration of steroids and antibiotics into human ears. Injection can be, for example, through the round window of the ear or through the cochlear capsule. Other inner ear administration methods are known in the art (see, e.g., Salt and Plontke, Drug Discovery Today, 10:1299-1306, 2005). In one embodiment, a catheter or pump can be positioned, e.g., in the ear (e.g., the outer, middle, and/or inner ear) of a patient during a surgical procedure. In one embodiment, a catheter or pump can be positioned, e.g., in the ear (e.g., the outer, middle, and/or inner ear) of a patient without the need for a surgical procedure.

In general, the cell therapy methods described in US Patent Publication No. 20120328580 can be used to promote complete or partial differentiation of a cell to or towards a mature cell type of the inner ear (e.g., a hair cell) in vitro. Cells resulting from such methods can then be transplanted or implanted into a patient in need of such treatment. The cell culture methods required to practice these methods, including methods for identifying and selecting suitable cell types, methods for promoting complete or partial differentiation of selected cells, methods for identifying complete or partially differentiated cell types, and methods for implanting complete or partially differentiated cells are described below.

Cells suitable for use in the present invention include, but are not limited to, cells that are capable of differentiating completely or partially into a mature cell of the inner ear, e.g., a hair cell (e.g., an inner and/or outer hair cell), when contacted, e.g., in vitro, with one or more of the compounds described herein. Exemplary cells that are capable of differentiating into a hair cell include, but are not limited to stem cells (e.g., inner ear stem cells, adult stem cells, bone marrow derived stem cells, embryonic stem cells, mesenchymal stem cells, skin stem cells, iPS cells, and fat derived stem cells), progenitor cells (e.g., inner ear progenitor cells), support cells (e.g., Deiters' cells, pillar cells, inner phalangeal cells, tectal cells and Hensen's cells), and/or germ cells. The use of stem cells for the replacement of inner ear sensory cells is described in Li et al., (U.S. Patent Publication No. 2005/0287127) and Li et al., (U.S. patent application Ser. No. 11/953,797). The use of bone marrow derived stem cells for the replacement of inner ear sensory cells is described in Edge et al., PCT/US2007/084654. iPS cells are described, e.g., at Takahashi et al., Cell, Volume 131, Issue 5, Pages 861-872(2007); Takahashi and Yamanaka, Cell 126, 663-76(2006); Okita et al., Nature 448, 260-262(2007); Yu, J. et al., Science 318(5858): 1917-1920(2007); Nakagawa et al., Nat. Biotechnol. 26:101-106(2008); and Zaehres and Scholer, Cell 131(5): 834-835(2007). Such suitable cells can be identified by analyzing (e.g., qualitatively or quantitatively) the presence of one or more tissue specific genes. For example, gene expression can be detected by detecting the protein product of one or more tissue-specific genes. Protein detection techniques involve staining proteins (e.g., using cell extracts or whole cells) using antibodies against the appropriate antigen. In this case, the appropriate antigen is the protein product of the tissue-specific gene expression. Although, in principle, a first antibody (i.e., the antibody that binds the antigen) can be labeled, it is more common (and improves the visualization) to use a second antibody directed against the first (e.g., an anti-IgG). This second antibody is conjugated either with fluorochromes, or appropriate enzymes for colorimetric reactions, or gold beads (for electron microscopy), or with the biotin-avidin system, so that the location of the primary antibody, and thus the antigen, can be recognized.

The composition and system may be delivered to the ear by direct application of pharmaceutical composition to the outer ear, with compositions modified from US Patent Publication No. 20110142917. In one embodiment the pharmaceutical composition is applied to the ear canal. Delivery to the ear may also be referred to as aural or otic delivery.

In one embodiment, the compositions, systems, or components thereof and/or vectors or vector systems can be delivered to ear via a transfection to the inner ear through the intact round window by a novel proteidic delivery technology which may be applied to the TnpB system of the present invention (see, e.g., Qi et al., Gene Therapy (2013), 1-9). About 40 μl of 10 mM RNA may be contemplated as the dosage for administration to the ear.

According to Rejali et al. (Hear Res. 2007 June; 228(1-2): 180-7), cochlear implant function can be improved by good preservation of the spiral ganglion neurons, which are the target of electrical stimulation by the implant and brain derived neurotrophic factor (BDNF) has previously been shown to enhance spiral ganglion survival in experimentally deafened ears. Rejali et al. tested a modified design of the cochlear implant electrode that includes a coating of fibroblast cells transduced by a viral vector with a BDNF gene insert. To accomplish this type of ex vivo gene transfer, Rejali et al. transduced guinea pig fibroblasts with an adenovirus with a BDNF gene cassette insert, and determined that these cells secreted BDNF and then attached BDNF-secreting cells to the cochlear implant electrode via an agarose gel, and implanted the electrode in the scala tympani. Rejali et al. determined that the BDNF expressing electrodes were able to preserve significantly more spiral ganglion neurons in the basal turns of the cochlea after 48 days of implantation when compared to control electrodes and demonstrated the feasibility of combining cochlear implant therapy with ex vivo gene transfer for enhancing spiral ganglion neuron survival. Such a system may be applied to the TnpB system of the present invention for delivery to the ear.

In one embodiment, the system set forth in Mukherjea et al. (Antioxidants & Redox Signaling, Volume 13, Number 5, 2010) can be adapted for transtympanic administration of the composition, system, or component thereof to the ear. In one embodiment, a dosage of about 2 mg to about 4 mg of TnpB polypeptide for administration to a human.

In one embodiment, the system set forth in [Jung et al. (Molecular Therapy, vol. 21 no. 4, 834-841 Apr. 2013) can be adapted for vestibular epithelial delivery of the composition, system, or component thereof to the ear. In one embodiment, a dosage of about 1 to about 30 mg of TnpB polypeptide for administration to a human.

### Treating Diseases in Non-Dividing Cells

In one embodiment, the gene or transcript to be corrected is in a non-dividing cell. Exemplary non-dividing cells are muscle cells or neurons. Non-dividing (especially non-dividing, fully differentiated) cell types present issues for gene targeting or genome engineering, for example because homologous recombination (HR) is generally suppressed in the G1 cell-cycle phase. However, while studying the mechanisms by which cells control normal DNA repair systems, Durocher discovered a previously unknown switch that keeps HR “off” in non-dividing cells and devised a strategy to toggle this switch back on. Orthwein et al. (Daniel Durocher's lab at the Mount Sinai Hospital in Ottawa, Canada) recently reported (Nature 16142, published online 9 Dec. 2015) have shown that the suppression of HR can be lifted and gene targeting successfully concluded in both kidney (293T) and osteosarcoma (U2OS) cells. Tumor suppressors, BRCA1, PALB2 and BRAC2 are known to promote DNA DSB repair by HR. They found that formation of a complex of BRCA1 with PALB2-BRAC2 is governed by a ubiquitin site on PALB2, such that action on the site by an E3 ubiquitin ligase. This E3 ubiquitin ligase is composed of KEAP1 (a PALB2-interacting protein) in complex with cullin-3 (CUL3)-RBX1. PALB2 ubiquitylation suppresses its interaction with BRCA1 and is counteracted by the deubiquitylase USP11, which is itself under cell cycle control. Restoration of the BRCA1-PALB2 interaction combined with the activation of DNA-end resection is sufficient to induce homologous recombination in G1, as measured by a number of methods including a Cas polypeptide nuclease-based gene-targeting assay directed at USP11 or KEAP1 (expressed from a pX459 vector). However, when the BRCA1-PALB2 interaction was restored in resection-competent G1 cells using either KEAP1 depletion or expression of the PALB2-KR mutant, a robust increase in gene-targeting events was detected. These teachings can be adapted for and/or applied to the compositions, systems, described herein.

Thus, reactivation of HR in cells, especially non-dividing, fully differentiated cell types is preferred, In one embodiment. In one embodiment, promotion of the BRCA1-PALB2 interaction is preferred. In one embodiment, the target cell is a non-dividing cell. In one embodiment, the target cell is a neuron or muscle cell. In one embodiment, the target cell is targeted in vivo. In one embodiment, the cell is in G1 and HR is suppressed. In one embodiment, use of KEAP1 depletion, for example inhibition of expression of KEAP1 activity, is preferred. KEAP1 depletion may be achieved through siRNA, for example as shown in Orthwein et al. Alternatively, expression of the PALB2-KR mutant (lacking all eight Lys residues in the BRCA1-interaction domain is preferred, either in combination with KEAP1 depletion or alone. PALB2-KR interacts with BRCA1 irrespective of cell cycle position. Thus, promotion or restoration of the BRCA1-PALB2 interaction, especially in G1 cells, is preferred. In one embodiment, especially where the target cells are non-dividing, or where removal and return (ex vivo gene targeting) is problematic, for example neuron or muscle cells. KEAP1 siRNA is available from ThermoFischer. In one embodiment, a BRCA1-PALB2 complex may be delivered to the G1 cell. In one embodiment, PALB2 deubiquitylation may be promoted for example by increased expression of the deubiquitylase USP11, so it is envisaged that a construct may be provided to promote or up-regulate expression or activity of the deubiquitylase USP11.

### Treating Diseases of the Eye

In one embodiment, the disease to be treated is a disease that affects the eyes. Thus, In one embodiment, the composition, system, or component thereof described herein is delivered to one or both eyes.

The composition, system, can be used to correct ocular defects that arise from several genetic mutations further described in Genetic Diseases of the Eye, Second Edition, edited by Elias I. Traboulsi, Oxford University Press, 2012.

In one embodiment, the condition to be treated or targeted is an eye disorder. In one embodiment, the eye disorder may include glaucoma. In one embodiment, the eye disorder includes a retinal degenerative disease. In one embodiment, the retinal degenerative disease is selected from Stargardt disease, Bardet-Biedl Syndrome, Best disease, Blue Cone Monochromacy, Choroidermia, Cone-rod dystrophy, Congenital Stationary Night Blindness, Enhanced S-Cone Syndrome, Juvenile X-Linked Retinoschisis, Leber Congenital Amaurosis, Malattia Leventinesse, Norrie Disease or X-linked Familial Exudative Vitreoretinopathy, Pattern Dystrophy, Sorsby Dystrophy, Usher Syndrome, Retinitis Pigmentosa, Achromatopsia or Macular dystrophies or degeneration, Retinitis Pigmentosa, Achromatopsia, and age related macular degeneration. In one embodiment, the retinal degenerative disease is Leber Congenital Amaurosis (LCA) or Retinitis Pigmentosa. Other exemplary eye diseases are described in greater detail elsewhere herein.

In one embodiment, the composition, system, is delivered to the eye, optionally via intravitreal injection or subretinal injection. Intraocular injections may be performed with the aid of an operating microscope. For subretinal and intravitreal injections, eyes may be prolapsed by gentle digital pressure and fundi visualized using a contact lens system consisting of a drop of a coupling medium solution on the cornea covered with a glass microscope slide coverslip. For subretinal injections, the tip of a 10-mm 34-gauge needle, mounted on a 5-μl Hamilton syringe may be advanced under direct visualization through the superior equatorial sclera tangentially towards the posterior pole until the aperture of the needle was visible in the subretinal space. Then, 2 μl of vector suspension may be injected to produce a superior bullous retinal detachment, thus confirming subretinal vector administration. This approach creates a self-sealing sclerotomy allowing the vector suspension to be retained in the subretinal space until it is absorbed by the RPE, usually within 48 h of the procedure. This procedure may be repeated in the inferior hemisphere to produce an inferior retinal detachment. This technique results in the exposure of approximately 70% of neurosensory retina and RPE to the vector suspension. For intravitreal injections, the needle tip may be advanced through the sclera 1 mm posterior to the corneoscleral limbus and 2 μl of vector suspension injected into the vitreous cavity. For intracameral injections, the needle tip may be advanced through a corneoscleral limbal paracentesis, directed towards the central cornea, and 2 μl of vector suspension may be injected. For intracameral injections, the needle tip may be advanced through a corneoscleral limbal paracentesis, directed towards the central cornea, and 2 μl of vector suspension may be injected. These vectors may be injected at titers of either 1.0-1.4×10¹⁰ or 1.0-1.4×10⁹ transducing units (TU)/ml.

In one embodiment, for administration to the eye, lentiviral vectors. In one embodiment, the lentiviral vector is an equine infectious anemia virus (EIAV) vector. Exemplary EIAV vectors for eye delivery are described in Balagaan, J Gene Med 2006; 8:275-285, Published online 21 Nov. 2005 in Wiley InterScience (www.interscience.wiley.com). DOI: 10.1002/jgm.845; Binley et al., HUMAN GENE THERAPY 23:980-991 (September 2012), which can be adapted for use with the composition, system, described herein. In one embodiment, the dosage can be 1.1×105 transducing units per eye (TU/eye) in a total volume of 100 μl.

Other viral vectors can also be used for delivery to the eye, such as AAV vectors, such as those described in Campochiaro et al., Human Gene Therapy 17:167-176 (February 2006), Millington-Ward et al. (Molecular Therapy, vol. 19 no. 4, 642-649 Apr. 2011; Dalkara et al. (Sci Transl Med 5, 189ra76(2013)), which can be adapted for use with the composition, system, described herein. In one embodiment, the dose can range from about 106 to 109.5 particle units. In the context of the Millington-Ward AAV vectors, a dose of about 2×1011 to about 6×1013 virus particles can be administered. In the context of Dalkara vectors, a dose of about 1×1015 to about 1×1016 vg/ml administered to a human.

In one embodiment, the sd-rxRNA® system of RXi Pharmaceuticals may be used/and or adapted for delivering composition, system, to the eye. In this system, a single intravitreal administration of 3 μg of sd-rxRNA results in sequence-specific reduction of PPIB mRNA levels for 14 days. The sd-rxRNA® system may be applied to the TnpB system of the present invention, contemplating a dose of about 3 to 20 mg of composition administered to a human.

In other embodiments, the methods of US Patent Publication No. 20130183282, which is directed to methods of cleaving a target sequence from the human rhodopsin gene, may also be modified to the TnpB system of the present invention.

In other embodiments, the methods of US Patent Publication No. 20130202678 for treating retinopathies and sight-threatening ophthalmologic disorders relating to delivering of the Puf-A gene (which is expressed in retinal ganglion and pigmented cells of eye tissues and displays a unique anti-apoptotic activity) to the sub-retinal or intravitreal space in the eye may be used or adapted. In particular, desirable targets are zgc: 193933, prdmla, spata2, tex10, rbb4, ddx3, zp2.2, Blimp-1 and HtrA2, all of which may be targeted by the composition, system, of the present invention.

Wu (Cell Stem Cell, 13:659-62, 2013) designed a guide RNA that led Cas9 to a single base pair mutation that causes cataracts in mice, where it induced DNA cleavage. Then using either the other wild-type allele or oligos given to the zygotes repair mechanisms corrected the sequence of the broken allele and corrected the cataract-causing genetic defect in mutant mouse. This approach can be adapted to and/or applied to the TnpB compositions, systems, described herein.

US Patent Publication No. 20120159653, describes use of zinc finger nucleases to genetically modify cells, animals and proteins associated with macular degeneration (MD), the teachings of which can be applied to and/or adapted for the TnpB compositions, systems, described herein.

One aspect of US Patent Publication No. 20120159653 relates to editing of any chromosomal sequences that encode proteins associated with MD which may be applied to the TnpB system of the present invention.

### Treating Muscle Diseases and Cardiovascular Diseases

In one embodiment, the composition, system can be used to treat and/or prevent a muscle disease and associated circulatory or cardiovascular disease or disorder. The present invention also contemplates delivering the composition, system, described herein, e.g., TnpB effector protein systems, to the heart. For the heart, a myocardium tropic adeno-associated virus (AAVM) is preferred, in particular AAVM41 which showed preferential gene transfer in the heart (see, e.g., Lin-Yanga et al., PNAS, Mar. 10, 2009, vol. 106, no. 10). Administration may be systemic or local. A dosage of about 1-10×1014 vector genomes is contemplated for systemic administration. See also, e.g., Eulalio et al. (2012) Nature 492:376 and Somasuntharam et al. (2013) Biomaterials 34:7790, the teachings of which can be adapted for and/or applied to the compositions, systems, described herein.

For example, US Patent Publication No. 20110023139, the teachings of which can be adapted for and/or applied to the compositions, systems, described herein describes use of zinc finger nucleases to genetically modify cells, animals and proteins associated with cardiovascular disease. Cardiovascular diseases generally include high blood pressure, heart attacks, heart failure, and stroke and TIA. Any chromosomal sequence involved in cardiovascular disease or the protein encoded by any chromosomal sequence involved in cardiovascular disease may be utilized in the methods described in this disclosure. The cardiovascular-related proteins are typically selected based on an experimental association of the cardiovascular-related protein to the development of cardiovascular disease. For example, the production rate or circulating concentration of a cardiovascular-related protein may be elevated or depressed in a population having a cardiovascular disorder relative to a population lacking the cardiovascular disorder. Differences in protein levels may be assessed using proteomic techniques including but not limited to Western blot, immunohistochemical staining, enzyme linked immunosorbent assay (ELISA), and mass spectrometry. Alternatively, the cardiovascular-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR).

The compositions, systems, herein can be used for treating diseases of the muscular system. The present invention also contemplates delivering the composition, system, described herein, effector protein systems, to muscle(s).

In one embodiment, the muscle disease to be treated is a muscle dystrophy such as DMD. In one embodiment, the composition, system, such as a system capable of RNA modification, described herein can be used to achieve exon skipping to achieve correction of the diseased gene. As used herein, the term “exon skipping” refers to the modification of pre-mRNA splicing by the targeting of splice donor and/or acceptor sites within a pre-mRNA with one or more complementary antisense oligonucleotide(s) (AONs). By blocking access of a spliceosome to one or more splice donor or acceptor site, an AON may prevent a splicing reaction thereby causing the deletion of one or more exons from a fully-processed mRNA. Exon skipping may be achieved in the nucleus during the maturation process of pre-mRNAs. In some examples, exon skipping may include the masking of key sequences involved in the splicing of targeted exons by using a composition, system, described herein capable of RNA modification. In one embodiment, exon skipping can be achieved in dystrophin mRNA. In one embodiment, the composition, system, can induce exon skipping at exon 1, 2, 3, 4, 5, 6, 7, 8, 10, 11, 12, 13, 14, 15, 16, 17, 18, 19, 20, 21, 22, 23, 24, 25, 26, 27, 28, 29, 30, 31, 32, 33, 34, 45, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or any combination thereof of the dystrophin mRNA. In one embodiment, the composition, system, can induce exon skipping at exon 43, 44, 50, 51, 52, 55, or any combination thereof of the dystrophin mRNA. Mutations in these exons, can also be corrected using non-exon skipping polynucleotide modification methods.

In one embodiment, for treatment of a muscle disease, the method of Bortolanza et al. Molecular Therapy vol. 19 no. 11, 2055-264 Nov. 2011) may be applied to an AAV expressing TnpB polypeptide and injected into humans at a dosage of about 2×10¹⁵ or 2×10¹⁶ vg of vector. The teachings of Bortolanza et al., can be adapted for and/or applied to the compositions, systems, described herein.

In one embodiment, the method of Dumonceaux et al. (Molecular Therapy vol. 18 no. 5, 881-887 May 2010) may be applied to an AAV expressing TnpB polypeptide and injected into humans, for example, at a dosage of about 1014 to about 1015 vg of vector. The teachings of Dumonceaux described herein can be adapted for and/or applied to the compositions, systems, described herein.

In one embodiment, the method of Kinouchi et al. (Gene Therapy (2008) 15, 1126-1130) may be applied to compositions described herein and injected into a human, for example, at a dosage of about 500 to 1000 ml of a 40 μM solution into the muscle.

In one embodiment, the method of Hagstrom et al. (Molecular Therapy Vol. 10, No. 2, August 2004) can be adapted for and/or applied to the compositions, systems, herein and injected at a dose of about 15 to about 50 mg into the great saphenous vein of a human.

In one embodiment, the method comprises treating a sickle cell related disease, e.g., sickle cell trait, sickle cell disease such as sickle cell anemia, β-thalassaemia. For example, the method and system may be used to modify the genome of the sickle cell, e.g., by correcting one or more mutations of the β-globin gene. In the case of β-thalassaemia, sickle cell anemia can be corrected by modifying HSCs with the systems. The system allows the specific editing of the cell's genome by cutting its DNA and then letting it repair itself. The TnpB polypeptide is inserted and directed by a nucleic acid component molecule to the mutated point and then it cuts the DNA at that point. Simultaneously, a healthy version of the sequence is inserted. This sequence is used by the cell's own repair system to fix the induced cut. In this way, the TnpB polypeptide allows the correction of the mutation in the previously obtained stem cells. The methods and systems may be used to correct HSCs as to sickle cell anemia using a system that targets and corrects the mutation (e.g., with a suitable HDR template that delivers a coding sequence for β-globin, advantageously non-sickling β-globin); specifically, the nucleic acid component molecule can target mutation that give rise to sickle cell anemia, and the HDR can provide coding for proper expression of β-globin. An nucleic acid component molecule that targets the mutation-and-TnpB polypeptide containing particle is contacted with HSCs carrying the mutation. The particle also can contain a suitable HDR template to correct the mutation for proper expression of β-globin; or the HSC can be contacted with a second particle or a vector that contains or delivers the HDR template. The so contacted cells can be administered; and optionally treated/expanded; cf. Cartier. The HDR template can provide for the HSC to express an engineered β-globin gene (e.g., BA-T87Q), or β-globin.

### Treating Diseases of the Liver and Kidney

In one embodiment, the composition, system, or component thereof described herein can be used to treat a disease of the kidney or liver. Thus, in one embodiment, delivery of the composition or component thereof described herein is to the liver or kidney.

Delivery strategies to induce cellular uptake of the therapeutic nucleic acid include physical force or vector systems such as viral-, lipid- or complex-based delivery, or nanocarriers. From the initial applications with less possible clinical relevance, when nucleic acids were addressed to renal cells with hydrodynamic high-pressure injection systemically, a wide range of gene therapeutic viral and non-viral carriers have been applied already to target posttranscriptional events in different animal kidney disease models in vivo (Csaba Révész and Péter Hamar (2011). Delivery Methods to Target RNAs in the Kidney, Gene Therapy Applications, Prof. Chunsheng Kang (Ed.), ISBN: 978-953-307-541-9, InTech, Available from: www.intechopen.com/books/gene-therapy-applications/delivery-methods-to-target-rnas-inthe-kidney). Delivery methods to the kidney may include those in Yuan et al. (Am J Physiol Renal Physiol 295: F605-F617, 2008). The method of Yuang et al. may be applied to the composition of the present invention contemplating a 1-2 g subcutaneous injection of polypeptide nuclease conjugated with cholesterol to a human for delivery to the kidneys. In one embodiment, the method of Molitoris et al. (J Am Soc Nephrol 20:1754-1764, 2009) can be adapted to the composition and a cumulative dose of 12-20 mg/kg to a human can be used for delivery to the proximal tubule cells of the kidneys. In one embodiment, the methods of Thompson et al. (Nucleic Acid Therapeutics, Volume 22, Number 4, 2012) can be adapted to the compositions and a dose of up to 25 mg/kg can be delivered via i.v. administration. In one embodiment, the method of Shimizu et al. (J Am Soc Nephrol 21:622-633, 2010) can be adapted to the compositions and a dose of about of 10-20 μmol compositions complexed with nanocarriers in about 1-2 liters of a physiologic fluid for i.p. administration can be used.

Other various delivery vehicles can be used to deliver the composition, system to the kidney such as viral, hydrodynamic, lipid, polymer nanoparticles, aptamers and various combinations thereof (see e.g., Larson et al., Surgery, (August 2007), Vol. 142, No. 2, pp. (262-269); Hamar et al., Proc Natl Acad Sci, (October 2004), Vol. 101, No. 41, pp. (14883-14888); Zheng et al., Am J Pathol, (October 2008), Vol. 173, No. 4, pp. (973-980); Feng et al., Transplantation, (May 2009), Vol. 87, No. 9, pp. (1283-1289); Q. Zhang et al., PloS ONE, (July 2010), Vol. 5, No. 7, e11709, pp. (1-13); Kushibikia et al., J Controlled Release, (July 2005), Vol. 105, No. 3, pp. (318-331); Wang et al., Gene Therapy, (July 2006), Vol. 13, No. 14, pp. (1097-1103); Kobayashi et al., Journal of Pharmacology and Experimental Therapeutics, (February 2004), Vol. 308, No. 2, pp. (688-693); Wolfrum et al., Nature Biotechnology, (September 2007), Vol. 25, No. 10, pp. (1149-1157); Molitoris et al., J Am Soc Nephrol, (August 2009), Vol. 20, No. 8 pp. (1754-1764); Mikhaylova et al., Cancer Gene Therapy, (March 2011), Vol. 16, No. 3, pp. (217-226); Y. Zhang et al., J Am Soc Nephrol, (April 2006), Vol. 17, No. 4, pp. (1090-1101); Singhal et al., Cancer Res, (May 2009), Vol. 69, No. 10, pp. (4244-4251); Malek et al., Toxicology and Applied Pharmacology, (April 2009), Vol. 236, No. 1, pp. (97-108); Shimizu et al., J Am Soc Nephrology, (April 2010), Vol. 21, No. 4, pp. (622-633); Jiang et al., Molecular Pharmaceutics, (May-June 2009), Vol. 6, No. 3, pp. (727-737); Cao et al, J Controlled Release, (June 2010), Vol. 144, No. 2, pp. (203-212); Ninichuk et al., Am J Pathol, (March 2008), Vol. 172, No. 3, pp. (628-637); Purschke et al., Proc Natl Acad Sci, (March 2006), Vol. 103, No. 13, pp. (5173-5178).

In one embodiment, delivery is to liver cells. In one embodiment, the liver cell is a hepatocyte. Delivery of the composition and system herein may be via viral vectors, especially AAV (and in particular AAV2/6) vectors. These can be administered by intravenous injection. A preferred target for the liver, whether in vitro or in vivo, is the albumin gene. This is a so-called ‘safe harbor” as albumin is expressed at very high levels and so some reduction in the production of albumin following successful gene editing is tolerated. It is also preferred as the high levels of expression seen from the albumin promoter/enhancer allows for useful levels of correct or transgene production (from the inserted recombination template) to be achieved even if only a small fraction of hepatocytes is edited. See sites identified by Wechsler et al. (reported at the 57th Annual Meeting and Exposition of the American Society of Hematology-abstract available online at ash.confex.com/ash/2015/webprogram/Paper86495.html and presented on 6^{th} December 2015) which can be adapted for use with the compositions, systems, herein.

Exemplary liver and kidney diseases that can be treated and/or prevented are described elsewhere herein.

### Treating Epithelial and Lung Diseases

In one embodiment, the disease treated or prevented by the composition and system described herein can be a lung or epithelial disease. The compositions and systems described herein can be used for treating epithelial and/or lung diseases. The present invention also contemplates delivering the composition, system, described herein, to one or both lungs.

In one embodiment, a viral vector can be used to deliver the composition, system, or component thereof to the lungs. In one embodiment, the AAV is an AAV-1, AAV-2, AAV-5, AAV-6, and/or AAV-9 for delivery to the lungs. (see, e.g., Li et al., Molecular Therapy, vol. 17 no. 12, 2067-277 Dec. 2009). In one embodiment, the MOI can vary from 1×10³ to 4×10⁵ vector genomes/cell. In one embodiment, the delivery vector can be an RSV vector as in Zamora et al. (Am J Respir Crit Care Med Vol 183. pp 531-538, 2011. The method of Zamora et al. may be applied to the TnpB system of the present invention and an aerosolized composition, for example with a dosage of 0.6 mg/kg, may be contemplated for the present invention.

Subjects treated for a lung disease may for example receive pharmaceutically effective amount of aerosolized AAV vector system per lung endobronchially delivered while spontaneously breathing. As such, aerosolized delivery is preferred for AAV delivery in general. An adenovirus or an AAV particle may be used for delivery. Suitable gene constructs, each operably linked to one or more regulatory sequences, may be cloned into the delivery vector. In this instance, the following constructs are provided as examples: Cbh or EF1a promoter for TnpB, U6 or H1 promoter for nucleic acid component molecule): A preferred arrangement is to use a CFTRdelta508 targeting nucleic acid component molecule, a repair template for deltaF508 mutation and a codon optimized composition, with optionally one or more nuclear localization signal or sequence(s) (NLS(s)), e.g., two (2) NLSs.

### Treating Diseases of the Skin

The compositions and systems described herein can be used for the treatment of skin diseases. The present invention also contemplates delivering the composition and system, described herein, to the skin.

In one embodiment, delivery to the skin (intradermal delivery) of the composition, system, or component thereof can be via one or more microneedles or microneedle containing device. For example, in one embodiment the device and methods of Hickerson et al. (Molecular Therapy—Nucleic Acids (2013) 2, e129) can be used and/or adapted to deliver the composition, system, described herein, for example, at a dosage of up to 300 μl of 0.1 mg/ml compositions to the skin.

In one embodiment, the methods and techniques of Leachman et al. (Molecular Therapy, vol. 18 no. 2, 442-446 February 2010) can be used and/or adapted for delivery of a compositions described herein to the skin.

In one embodiment, the methods and techniques of Zheng et al. (PNAS, Jul. 24, 2012, vol. 109, no. 30, 11975-11980) can be used and/or adapted for nanoparticle delivery of a compositions described herein to the skin. In one embodiment, as dosage of about 25 nM applied in a single application can achieve gene knockdown in the skin.

### Treating Cancer

The compositions, systems, described herein can be used for the treatment of cancer. The present invention also contemplates delivering the composition, system, described herein, to a cancer cell. Also, as is described elsewhere herein the compositions, systems, can be used to modify an immune cell, such as a CAR or CAR T cell, which can then in turn be used to treat and/or prevent cancer. This is also described in International Patent Publication No. WO 2015/161276, the disclosure of which is hereby incorporated by reference and described herein below.

Target genes suitable for the treatment or prophylaxis of cancer can include those set forth in Tables 2 and 3. In one embodiment, target genes for cancer treatment and prevention can also include those described in International Patent Publication No. WO 2015/048577 the disclosure of which is hereby incorporated by reference and can be adapted for and/or applied to the composition, system, described herein.

### Adoptive Cell Therapy

The compositions, systems, and components thereof described herein can be used to modify cells for an adoptive cell therapy. In an aspect of the invention, methods and compositions which involve editing a target nucleic acid sequence, or modulating expression of a target nucleic acid sequence, and applications thereof in connection with cancer immunotherapy are comprehended by adapting the composition, system, of the present invention. In some examples, the compositions, systems, and methods may be used to modify a stem cell (e.g., induced pluripotent cell) to derive modified natural killer cells, gamma delta T cells, and alpha beta T cells, which can be used for the adoptive cell therapy. In certain examples, the compositions, systems, and methods may be used to modify modified natural killer cells, gamma delta T cells, and alpha beta T cells.

As used herein, “ACT”, “adoptive cell therapy” and “adoptive cell transfer” may be used interchangeably. In one embodiment, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1): 424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June; 24(6): 724-730; Besser et al., (2010) Clin. Cancer Res 16(9) 2646-55; Dudley et al., (2002) Science 298(5594): 850-4; and Dudley et al., (2005) Journal of Clinical Oncology 23(10): 2346-57.) or genetically re-directed peripheral blood mononuclear cells (Johnson et al., (2009) Blood 114(3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3(95): 95ra73). In one embodiment, allogenic cells immune cells are transferred (see, e.g., Ren et al., (2017) Clin Cancer Res 23(9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32:189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3): 453-62).

In one embodiment, an antigen (such as a tumor antigen) to be targeted in adoptive cell therapy (such as particularly CAR or TCR T-cell therapy) of a disease (such as particularly of tumor or cancer) may be selected from a group consisting of: MR1 (see, e.g., Crowther, et al., 2020, Genome-wide CRISPR-Cas9 screening reveals ubiquitous T cell cancer targeting via the monomorphic MHC class I-related protein MR1, Nature Immunology volume 21, pages 178-185), B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); K-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTAI (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac (2-8) aNeu5Ac (2-3) bDGalp (1-4) bDGlcp (1-1) Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis (Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac (2-3) bDGalp (1-4) bDGlcp (1-1) Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SARTI, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); LICAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In one embodiment, an antigen to be targeted in adoptive cell therapy (such as particularly CAR or TCR T-cell therapy) of a disease (such as particularly of tumor or cancer) is a tumor-specific antigen (TSA).

In one embodiment, an antigen to be targeted in adoptive cell therapy (such as particularly CAR or TCR T-cell therapy) of a disease (such as particularly of tumor or cancer) is a neoantigen.

In one embodiment, an antigen to be targeted in adoptive cell therapy (such as particularly CAR or TCR T-cell therapy) of a disease (such as particularly of tumor or cancer) is a tumor-associated antigen (TAA).

In one embodiment, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (DI), and any combinations thereof.

In one embodiment, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR a and B chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO 9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, In one embodiment, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signaling domains of either CD3% or FcRγ (scFv-CD35 or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD32; see U.S. Pat. Nos. 8,911,993; 8,916,381; 8,975,071; 9,101,584; 9,102,760; 9,102,761). Third-generation CARs include a combination of costimulatory endodomains, such a CD32-chain, CD97, GDI la-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD35 or scFv-CD28-OX40-CD32; see U.S. Pat. Nos. 8,906,682; 8,399,645; 5,686,281; PCT Publication No. WO 2014/134165; PCT Publication No. WO 2012/079000). In one embodiment, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon R1b), CD79a, CD79b, Fc gamma RIIa, DAP10, and DAP12. In certain preferred embodiments, the primary signaling domain comprises a functional signaling domain of CD35 or FcRγ. In one embodiment, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAMI (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In one embodiment, the one or more costimulatory signaling domains comprise a functional signaling domain of a protein selected, each independently, from the group consisting of: 4-1BB, CD27, and CD28. In one embodiment, a chimeric antigen receptor may have the design as described in U.S. Pat. No. 7,446,190, comprising an intracellular domain of CD35 chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139. Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3 chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native aßTCR, for example by antigen on professional antigen-presenting cells, with attendant costimulation. In addition, additional engineered receptors may be provided on the immunoresponsive cells, for example to improve targeting of a T-cell attack and/or minimize side effects.

By means of an example and without limitation, Kochenderfer et al., (2009) J Immunother. 32(7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34:1157-1165), a portion of the human CD28 molecule, and the intracellular component of the human TCR-molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-molecule. The exact sequence of the CD28 molecule included in the FMC63-28Z CAR corresponded to Genbank identifier NM_006139; the sequence included all amino acids starting with the amino acid sequence IEVMYPPPY (SEQ. I.D. No. 234) and continuing all the way to the carboxy-terminus of the protein. To encode the anti-CD19 scFv component of the vector, the authors designed a DNA sequence which was based on a portion of a previously published CAR (Cooper et al., (2003) Blood 101:1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16:457-472) as well as part of the extracellular portion of human CD28, the entire transmembrane and cytoplasmic portion of human CD28, and the cytoplasmic portion of the human TCR-(molecule (as in Maher et al., 2002) Nature Biotechnology 20:70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in one embodiment, cells intended for adoptive cell therapies, more particularly immunoresponsive cells such as T cells, may express the FMC63-28Z CAR as described by Kochenderfer et al. (supra). Hence, in one embodiment, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD32 chain, and a costimulatory signaling region comprising a signaling domain of CD28. Preferably, the CD28 amino acid sequence is as set forth in Genbank identifier NM 006139 (sequence version 1, 2 or 3) starting with the amino acid sequence IEVMYPPPY (SEQ ID NO: 234) and continuing all the way to the carboxy-terminus of the protein. Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in International Patent Publication No. WO 2015/187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signaling domains (CD28-CD3ξ; 4-1BB-CD3ξ; CD27-CD3ξ; CD28-CD27-CD3ξ, 4-1BB-CD27-CD3ξ; CD27-4-1BB-CD3ξ; CD28-CD27-FcεRI gamma chain; or CD28-FcεRI gamma chain) were disclosed. Hence, in one embodiment, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signaling domain as set forth in Table 1 of International Application No. WO 2015/187528. Preferably, the antigen is CD19, more preferably the antigen-binding element is an anti-CD19 scFv, even more preferably the mouse or human anti-CD19 scFv as described in Example 1 of WO 2015/187528. In one embodiment, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1): 55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147:1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In one embodiment, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In one embodiment, the chimeric inhibitory receptor comprises an extracellular antigen-binding element (or portion or domain) configured to specifically bind to a target antigen, a transmembrane domain, and an intracellular immunosuppressive or repressive signaling domain. In one embodiment, the second target antigen is an antigen that is not expressed on the surface of a cancer cell or infected cell or the expression of which is downregulated on a cancer cell or an infected cell. In one embodiment, the second target antigen is an MHC-class I molecule. In one embodiment, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in one embodiment, TCR expression may eliminated using RNA interference (e.g., snucleic acid component, siRNA, miRNA, etc.), TnpB polypeptide, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US Patent Publication Nos. US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLOS One, 2013) or to downregulate expression of the CAR following binding to the target antigen (International Patent Publication No. WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2^{nd} generation antigen-specific CARs signaling through CD35 and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In one embodiment, ACT includes co-transferring CD4+Th1 cells and CD8+CTLs to induce a synergistic antitumor response (see, e.g., Li et al., Adoptive cell therapy with CD4+ T helper 1 cells and CD8+ cytotoxic T cells enhances complete rejection of an established tumor, leading to generation of endogenous memory responses to non-targeted tumor epitopes. Clin Transl Immunology. 2017 October; 6(10): e160).

In one embodiment, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2): 362-73; and Martin-Orozco N, et al., T helper 17 cells promote cytotoxic T cell activation in tumor immunity. Immunity. 2009 Nov. 20; 31(5): 787-98). Those studies involved an adoptive T cell transfer (ACT) therapy approach, which takes advantage of CD4+ T cells that express a TCR recognizing tyrosinase tumor antigen. Exploitation of the TCR leads to rapid expansion of Th17 populations to large numbers ex vivo for reinfusion into the autologous tumor-bearing hosts.

In one embodiment, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In one embodiment, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1): 56-71. doi: 10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In one embodiment, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10): 1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells, or population of cells, may be made resistant to at least one immunosuppressive agent due to the inactivation of a gene encoding a receptor for such immunosuppressive agent. In one embodiment, the immunosuppressive treatment provides for the selection and expansion of the immunoresponsive T cells within the patient.

In one embodiment, the treatment can be administered before primary treatment (e.g., surgery or radiation therapy) to shrink a tumor before the primary treatment. In another embodiment, the treatment can be administered after primary treatment to remove any remaining cancer cells.

In one embodiment, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In one embodiment, the disclosed CARs may be delivered or administered into a cavity formed by the resection of tumor tissue (i.e., intracavity delivery) or directly into a tumor prior to resection (i.e., intratumoral delivery). In one embodiment, the cell compositions of the present invention are preferably administered by intravenous injection.

The administration of the cells or population of cells can consist of the administration of 104-109 cells per kg body weight, preferably 105 to 106 cells/kg body weight including all integer values of cell numbers within those ranges. Dosing in CAR T cell therapies may for example involve administration of from 106 to 109 cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6:95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; International Patent Publication WO 2011/146862; International Patent Publication WO 2014/011987; International Patent Publication WO 2013/040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6): 1107-15(2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75(18): 3853; Ren et al., 2017, Multiplex genome editing to generate universal CAR T cells resistant to PD1 inhibition, Clin Cancer Res. 2017 May 1; 23(9): 2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25; 9(374); Legut, et al., 2018, CRISPR-mediated TCR replacement generates superior anticancer transgenic T cells. Blood, 131(3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. The composition and systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g., TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see International Patent Publication Nos. WO 2013/176915, WO 2014/059173, WO 2014/172606, WO 2014/184744, and WO 2014/191128).

In one embodiment, editing may result in inactivation of a gene. By inactivating a gene, it is intended that the gene of interest is not expressed in a functional protein form. In a particular embodiment, the system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In one embodiment, homology directed repair (HDR) is used to concurrently inactivate a gene (e.g., TRAC) and insert an endogenous TCR or CAR into the inactivated locus.

Hence, in one embodiment, editing of cells, particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543:113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, a and B, which assemble to form a heterodimer and associates with the CD3-transducing subunits to form the T cell receptor complex present on the cell surface. Each a and B chain of the TCR consists of an immunoglobulin-like N-terminal variable (V) and constant (C) region, a hydrophobic transmembrane domain, and a short cytoplasmic region. As for immunoglobulin molecules, the variable region of the a and B chains are generated by V (D) J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in one embodiment, editing of cells, particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as TnpB system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12): 4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In one embodiment, editing of cells, particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In one embodiment, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2): 356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

International Patent Publication No. WO 2014/172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In one embodiment, metallothioneins are targeted by gene editing in adoptively transferred T cells.

In one embodiment, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244(2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDMI, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, International Patent Publication No. WO 2016/196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as the composition or system herein) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIRI, CD160, 2B4, TGFR beta, CEACAM-1, CEACAM-3, or CEACAM-5. Ren et al., (2017) Clin Cancer Res 23(9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In one embodiment, cells may be engineered to express a CAR, wherein expression and/or function of methylcytosine dioxygenase genes (TET1, TET2 and/or TET3) in the cells has been reduced or eliminated, (such as the composition or system herein) (for example, as described in WO201704916).

In one embodiment, editing of cells, particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In one embodiment, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in International Patent Publication Nos. WO 2016/011210 and WO 2017/011804).

In one embodiment, editing of cells, particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23(9) 2255-2266 performed lentiviral delivery of CAR and electro-transfer of Cas mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In one embodiment, a cell may be multiplied edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In one embodiment, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In one embodiment of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed, and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (e.g., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In one embodiment, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In one embodiment, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead: cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In one embodiment, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells are minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In one embodiment, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g., via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274(5284): 94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of 125I labeled B2-microglobulin (B2m) into MHC class I/B2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003/057171, U.S. Pat. No. 8,034,334, and U.S. Patent Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In one embodiment, T cells comprising a CAR or an exogenous TCR, may be manufactured as described in International Patent Publication No. WO 2015/120096, by a method comprising enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In one embodiment, T cells comprising a CAR or an exogenous TCR, may be manufactured as described in WO 2015/120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In one embodiment, T cell maturation or differentiation in vitro may be delayed or inhibited by the method as described in International Patent Publication No. WO 2017/070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In one embodiment, a patient in need of a T cell therapy may be conditioned by a method as described in International Patent Publication No. WO 2016/191756 comprising administering to the patient a dose of cyclophosphamide between 200 mg/m2/day and 2000 mg/m2/day and a dose of fludarabine between 20 mg/m2/day and 900 mg/m2/day.

### Diseases

Genetic Diseases and Diseases with a Genetic and/or Epigenetic Aspect

The compositions, systems, or components thereof can be used to treat and/or prevent a genetic disease or a disease with a genetic and/or epigenetic aspect. The genes and conditions exemplified herein are not exhaustive. In one embodiment, a method of treating and/or preventing a genetic disease can include administering a composition, system, and/or one or more components thereof to a subject, where the composition, system, and/or one or more components thereof is capable of modifying one or more copies of one or more genes associated with the genetic disease or a disease with a genetic and/or epigenetic aspect in one or more cells of the subject. In one embodiment, modifying one or more copies of one or more genes associated with a genetic disease or a disease with a genetic and/or epigenetic aspect in the subject can eliminate a genetic disease or a symptom thereof in the subject. In one embodiment, modifying one or more copies of one or more genes associated with a genetic disease or a disease with a genetic and/or epigenetic aspect in the subject can decrease the severity of a genetic disease or a symptom thereof in the subject. In one embodiment, the compositions, systems, or components thereof can modify one or more genes or polynucleotides associated with one or more diseases, including genetic diseases and/or those having a genetic aspect and/or epigenetic aspect, including but not limited to, any one or more set forth in Table 4A. It will be appreciated that those diseases and associated genes listed herein are non-exhaustive and non-limiting. Further some genes play roles in the development of multiple diseases.

TABLE 4A
Exemplary Genetic and Other Diseases and Associated Genes

| | Primary | Additional | |
|---|---|---|---|
| | Tissues or | Tissues/ | |
| | System | Systems | |
| Disease Name | Affected | Affected | Genes |
| Achondroplasia | Bone and | | fibroblast growth factor receptor 3 |
| | Muscle | | (FGFR3) |
| Achromatopsia | eye | | CNGA3, CNGB3, GNAT2, PDE6C, |
| | | | PDE6H, ACHM2, ACHM3, |
| Acute Renal Injury | kidney | | NFkappaB, AATF, p85alpha, FAS, |
| | | | Apoptosis cascade elements (e.g., |
| | | | FASR, Caspase 2, 3, 4, 6, 7, 8, 9, 10, |
| | | | AKT, TNF alpha, IGF1, IGF1R, |
| | | | RIPK1), p53 |
| Age Related Macular | eye | | Abcr; CCL2; CC2; CP |
| Degeneration | | | (ceruloplasmin); Timp3; cathepsinD; |
| | | | VLDLR, CCR2 |
| AIDS | Immune System | | KIR3DL1, NKAT3, NKB1, AMB11, |
| | | | KIR3DS1, IFNG, CXCL12, SDF1 |
| Albinism (including | Skin, hair, eyes, | | TYR, OCA2, TYRP1, and SLC45A2, |
| oculocutaneous albinism (types | | | SLC24A5 and C10orf11 |
| 1-7) and ocular albinism) | | | |
| Alkaptonuria | Metabolism of | Tissues/organs | HGD |
| | amino acids | where | |
| | | homogentisic | |
| | | acid | |
| | | accumulates, | |
| | | particularly | |
| | | cartilage (joints), | |
| | | heart valves, | |
| | | kidneys | |
| alpha-1 antitrypsin deficiency | Lung | Liver, skin, | SERPINA1, those set forth in |
| (AATD or A1AD) | | vascular system, | WO2017165862, PiZ allele |
| | | kidneys, GI | |
| ALS | CNS | [0100] | SOD1; ALS2; ALS3; ALS5; |
| | | | ALS7; STEX; FUS; TARDBP; VEGF |
| | | | (VEGF-a; |
| | | | VEGF-b; VEGF-c); DPP6; NEFH, |
| | | | PTGS1, SLC1A2, TNFRSF10B, |
| | | | PRPH, HSP90AA1, CRIA2, IFNG, |
| | | | AMPA2 S100B, FGF2, AOX1, CS, |
| | | | TXN, RAPHJ1, MAP3K5, NBEAL1, |
| | | | GPX1, ICA1L, RAC1, MAPT, ITPR2, |
| | | | ALS2CR4, GLS, ALS2CR8, CNTFR, |
| | | | ALS2CR11, FOLH1, FAM117B, |
| | | | P4HB, CNTF, SQSTM1, STRADB, |
| | | | NAIP, NLR, YWHAQ, SLC33A1, |
| | | | TRAK2, SCA1, NIF3L1, NIF3, |
| | | | PARD3B, COX8A, CDK15, HECW1, |
| | | | HECT, C2, WW 15, NOS1, MET, |
| | | | SOD2, HSPB1, NEFL, CTSB, ANG, |
| | | | HSPA8, Rnase A, VAPB, VAMP, |
| | | | SNCA, alpha HGF, CAT, ACTB, |
| | | | NEFM, TH, BCL2, FAS, CASP3, |
| | | | CLU, SMN1, G6PD, BAX, HSF1, |
| | | | RNF19A, JUN, ALS2CR12, HSPA5, |
| | | | MAPK14, APEX1, TXNRD1, NOS2, |
| | | | TIMP1, CASP9, XIAP, GLG1, EPO, |
| | | | VEGFA, ELN, GDNF, NFE2L2, |
| | | | SLC6A3, HSPA4, APOE, PSMB8, |
| | | | DCTN2, TIMP3, KIFAP3, SLC1A1, |
| | | | SMN2, CCNC, STUB1, ALS2, |
| | | | PRDX6, SYP, CABIN1, CASP1, |
| | | | GART, CDK5, ATXN3, RTN4, |
| | | | C1QB, VEGFC, HTT, PARK7, XDH, |
| | | | GFAP, MAP2, CYCS, FCGR3B, CCS, |
| | | | UBL5, MMP9m SLC18A3, TRPM7, |
| | | | HSPB2, AKT1, DEERL1, CCL2, |
| | | | NGRN, GSR, TPPP3, APAF1, |
| | | | BTBD10, GLUD1, CXCR4, S: C1A3, |
| | | | FLT1, PON1, AR, LIF, ERBB3, |
| | | | : GA: S1, CD44, TP53, TLR3, GRIA1, |
| | | | GAPDH, AMPA, GRIK1, DES, |
| | | | CHAT, FLT4, CHMP2B, BAG1, |
| | | | CRNA COMPONENT4, GSS, BAK1, |
| | | | KDR, GSTP1, OGG1, IL6 |
| Alzheimer's Disease | Brain | | E1; CHIP; UCH; UBB; Tau; LRP; |
| | | | PICALM; CLU; PS1; |
| | | | SORL1; CR1; VLDLR; UBA1; |
| | | | UBA3; CHIP28; AQP1; UCHL1; |
| | | | UCHL3; APP, AAA, CVAP, AD1, |
| | | | APOE, AD2, DCP1, ACE1, MPO, |
| | | | PACIP1, PAXIP1L, PTIP, A2M, |
| | | | BLMH, BMH, PSEN1, AD3, ALAS2, |
| | | | ABCA1, BIN1, BDNF, BTNL8, |
| | | | C1ORF49, CDH4, CHRNB2, |
| | | | CKLFSF2, CLEC4E, CR1L, CSF3R, |
| | | | CST3, CYP2C, DAPK1, ESR1, |
| | | | FCAR, FCGR3B, FFA2, FGA, GAB2, |
| | | | GALP, GAPDHS, GMPB, HP, HTR7, |
| | | | IDE, IF127, IFI6, IFIT2, IL1RN, IL- |
| | | | 1RA, IL8RA, IL8RB, JAG1, KCNJ15, |
| | | | LRP6, MAPT, MARK4, MPHOSPH1, |
| | | | MTHFR, NBN, NCSTN, NIACR2, |
| | | | NMNAT3, NTM, ORM1, P2RY13, |
| | | | PBEF1, PCK1, PICALM, PLAU, |
| | | | PLXNC1, PRNP, PSEN1, PSEN2, |
| | | | PTPRA, RALGPS2, RGSL2, |
| | | | SELENBP1, SLC25A37, SORL1, |
| | | | Mitoferrin-1, TF, TFAM, TNF, |
| | | | TNFRSF10C, UBE1C |
| Amyloidosis | | | APOA1, APP, AAA, CVAP, AD1, |
| | | | GSN, FGA, LYZ, TTR, PALB |
| Amyloid neuropathy | | | TTR, PALB |
| Anemia | Blood | | CDAN1, CDA1, RPS19, DBA, PKLR, |
| | | | PK1, NT5C3, UMPH1, PSN1, RHAG, |
| | | | RH50A, NRAMP2, SPTB, ALAS2, |
| | | | ANH1, ASB, ABCB7, ABC7, ASAT |
| Angelman Syndrome | Nervous system, | | UBE3A |
| | brain | | |
| Attention Deficit Hyperactivity | Brain | | PTCHD1 |
| Disorder (ADHD) | | | |
| Autoimmune lymphoproliferative | Immune system | | TNFRSF6, APT1, FAS, CD95, |
| syndrome | | | ALPS1A |
| Autism, Autism spectrum | Brain | | PTCHD1; Mecp2; BZRAP1; MDGA2; |
| disorders (ASDs), including | | | Sema5A; Neurexin 1; GLO1, RTT, |
| Asperger's and a general | | | PPMX, MRX16, RX79, NLGN3, |
| diagnostic category called | | | NLGN4, KIAA1260, AUTSX2, |
| Pervasive Developmental | | | FMR1, FMR2; FXR1; FXR2; |
| Disorders (PDDs) | | | MGLUR5, ATP10C, CDH10, GRM6, |
| | | | MGLUR6, CDH9, CNTN4, NLGN2, |
| | | | CNTNAP2, SEMA5A, DHCR7, |
| | | | NLGN4X, NLGN4Y, DPP6, NLGN5, |
| | | | EN2, NRCAM, MDGA2, NRXN1, |
| | | | FMR2, AFF2, FOXP2, OR4M2, |
| | | | OXTR, FXR1, FXR2, PAH, |
| | | | GABRA1, PTEN, GABRA5, PTPRZ1, |
| | | | GABRB3, GABRG1, HIRIP3, |
| | | | SEZ6L2, HOXA1, SHANK3, IL6, |
| | | | SHBZRAP1, LAMB1, SLC6A4, |
| | | | SERT, MAPK3, TAS2R1, MAZ, |
| | | | TSC1, MDGA2, TSC2, MECP2, |
| | | | UBE3A, WNT2, see also |
| | | | 20110023145 |
| autosomal dominant polycystic | kidney | liver | PKD1, PKD2 |
| kidney disease (ADPKD) - | | | |
| (includes diseases such as von | | | |
| Hippel-Lindau disease and | | | |
| Tuberous sclerosis complex | | | |
| disease) | | | |
| Autosomal Recessive Polycystic | kidney | liver | PKDH1 |
| Kidney Disease (ARPKD) | | | |
| Ataxia-Telangiectasia (a.k.a | Nervous system, | various | ATM |
| Louis Bar syndrome) | immune system | | |
| B-Cell Non-Hodgkin Lymphoma | | | BCL7A, BCL7 |
| Bardet-Biedl syndrome | Eye, | Liver, ear, | ARL6, BBS1, BBS2, BBS4, BBS5, |
| | musculoskeletal | gastrointestinal | BBS7, BBS9, BBS10, BBS12, |
| | system, kidney, | system, brain | CEP290, INPP5E, LZTFL1, MKKS, |
| | reproductive | | MKS1, SDCCAG8, TRIM32, TTC8 |
| | organs | | |
| Bare Lymphocyte Syndrome | blood | | TAPBP, TPSN, TAP2, ABCB3, PSF2, |
| | | | RING11, MHC2TA, C2TA, RFX5, |
| | | | RFXAP, RFX5 |
| Bartter's Syndrome (types I, II, | kidney | | SLC12A1 (type I), KCNJ1 (type II), |
| III, IVA and B, and V) | | | CLCNKB (type III), BSND (type IV |
| | | | A), or both the CLCNKA CLCNKB |
| | | | genes (type IV B), CASR (type V). |
| Becker muscular dystrophy | Muscle | | DMD, BMD, MYF6 |
| Best Disease (Vitelliform | eye | | VMD2 |
| Macular Dystrophy type 2 ) | | | |
| Bleeding Disorders | blood | | TBXA2R, P2RX1, P2X1 |
| Blue Cone Monochromacy | eye | | OPN1LW, OPN1MW, and LCR |
| Breast Cancer | Breast tissue | | BRCA1, BRCA2, COX-2 |
| Bruton's Disease (aka X-linked | Immune system, | | BTK |
| Agammglobulinemia) | specifically B | | |
| | cells | | |
| Cancers (e.g., lymphoma, chronic | Various | | FAS, BID, CTLA4, PDCD1, CBLB, |
| lymphocytic leukemia (CLL), B | | | PTPN6, TRAC, TRBC, those |
| cell acute lymphocytic leukemia | | | described in WO2015048577 |
| (B-ALL), acute lymphoblastic | | | |
| leukemia, acute myeloid | | | |
| leukemia, non-Hodgkin's | | | |
| lymphoma (NHL), diffuse large | | | |
| cell lymphoma (DLCL), multiple | | | |
| myeloma, renal cell carcinoma | | | |
| (RCC), neuroblastoma, colorectal | | | |
| cancer, breast cancer, ovarian | | | |
| cancer, melanoma, sarcoma, | | | |
| prostate cancer, lung cancer, | | | |
| esophageal cancer, hepatocellular | | | |
| carcinoma, pancreatic cancer, | | | |
| astrocytoma, mesothelioma, head | | | |
| and neck cancer, and | | | |
| medulloblastoma | | | |
| Cardiovascular Diseases | heart | Vascular system | IL1B, XDH, TP53, PTGS, MB, IL4, |
| | | | ANGPT1, ABCGu8, CTSK, PTGIR, |
| | | | KCNJ11, INS, CRP, PDGFRB, |
| | | | CCNA2, PDGFB, KCNJ5, KCNN3, |
| | | | CAPN10, ADRA2B, ABCG5, |
| | | | PRDX2, CPAN5, PARP14, MEX3C, |
| | | | ACE, RNF, IL6, TNF, STN, |
| | | | SERPINE1, ALB, ADIPOQ, APOB, |
| | | | APOE, LEP, MTHFR, APOA1, |
| | | | EDN1, NPPB, NOS3, PPARG, PLAT, |
| | | | PTGS2, CETP, AGTR1, HMGCR, |
| | | | IGF1, SELE, REN, PPARA, PON1, |
| | | | KNG1, CCL2, LPL, VWF, F2, |
| | | | ICAM1, TGFB, NPPA, IL10, EPO, |
| | | | SOD1, VCAM1, IFNG, LPA, MPO, |
| | | | ESR1, MAPK, HP, F3, CST3, COG2, |
| | | | MMP9, SERPINC1, F8, HMOX1, |
| | | | APOC3, IL8, PROLI, CBS, NOS2, |
| | | | TLR4, SELP, ABCA1, AGT, LDLR, |
| | | | GPT, VEGFA, NR3C2, IL18, NOS1, |
| | | | NR3C1, FGB, HGF, IL1A, AKT1, |
| | | | LIPC, HSPD1, MAPK14, SPP1, |
| | | | ITGB3, CAT, UTS2, THBD, F10, CP, |
| | | | TNFRSF11B, EGFR, MMP2, PLG, |
| | | | NPY, RHOD, MAPK8, MYC, FN1, |
| | | | CMA1, PLAU, GNB3, ADRB2, |
| | | | SOD2, F5, VDR, ALOX5, HLA- |
| | | | DRB1, PARP1, CD40LG, PON2, |
| | | | AGER, IRS1, PTGS1, ECE1, F7, |
| | | | IRMN, EPHX2, IGFBP1, MAPK10, |
| | | | FAS, ABCB1, JUN, IGFBP3, CD14, |
| | | | PDE5A, AGTR2, CD40, LCAT, |
| | | | CCR5, MMP1, TIMP1, ADM, |
| | | | DYT10, STAT3, MMP3, ELN, USF1, |
| | | | CFH, HSPA4, MMP12, MME, F2R, |
| | | | SELL, CTSB, ANXA5, ADRB1, |
| | | | CYBA, FGA, GGT1, LIPG, HIF1A, |
| | | | CXCR4, PROC, SCARB1, CD79A, |
| | | | PLTP, ADD1, FGG, SAA1, KCNH2, |
| | | | DPP4, NPR1, VTN, KIAA0101, FOS, |
| | | | TLR2, PPIG, ILIR1, AR, CYP1A1, |
| | | | SERPINA1, MTR, RBP4, APOA4, |
| | | | CDKN2A, FGF2, EDNRB, ITGA2, |
| | | | VLA-2, CABIN1, SHBG, HMGB1, |
| | | | HSP90B2P, CYP3A4, GJA1, CAV1, |
| | | | ESR2, LTA, GDF15, BDNF, |
| | | | CYP2D6, NGF, SP1, TGIF1, SRC, |
| | | | EGF, PIK3CG, HLA-A, KCNQ1, |
| | | | CNR1, FBN1, CHKA, BEST1, |
| | | | CTNNB1, IL2, CD36, PRKAB1, TPO, |
| | | | ALDH7A1, CX3CR1, TH, F9, CH1, |
| | | | TF, HFE, IL17A, PTEN, GSTM1, |
| | | | DMD, GATA4, F13A1, TTR, FABP4, |
| | | | PON3, APOC1, INSR, TNFRSF1B, |
| | | | HTR2A, CSF3, CYP2C9, TXN, |
| | | | CYP11B2, PTH, CSF2, KDR, |
| | | | PLA2G2A, THBS1, GCG, RHOA, |
| | | | ALDH2, TCF7L2, NFE2L2, |
| | | | NOTCH1, UGT1A1, IFNA1, PPARD, |
| | | | SIRT1, GNHR1, PAPPA, ARR3, |
| | | | NPPC, AHSP, PTK2, IL13, MTOR, |
| | | | ITGB2, GSTT1, IL6ST, CPB2, |
| | | | CYP1A2, HNF4A, SLC64A, |
| | | | PLA2G6, TNFSF11, SLC8A1, F2RL1, |
| | | | AKR1A1, ALDH9A1, BGLAP, |
| | | | MTTP, MTRR, SULT1A3, RAGE, |
| | | | C4B, P2RY12, RNLS, CREB1, |
| | | | POMC, RAC1, LMNA, CD59, |
| | | | SCM5A, CYP1B1, MIF, MMP13, |
| | | | TIMP2, CYP19A1, CUP21A2, |
| | | | PTPN22, MYH14, MBL2, SELPLG, |
| | | | AOC3, CTSL1, PCNA, IGF2, ITGB1, |
| | | | CAST, CXCL12, IGHE, KCNE1, |
| | | | TFRC, COL1A1, COL1A2, IL2RB, |
| | | | PLA2G10, ANGPT2, PROCR, NOX4, |
| | | | HAMP, PTPN11, SLCA1, IL2RA, |
| | | | CCL5, IRF1, CF: AR, CA: CA, EIF4E, |
| | | | GSTP1, JAK2, CYP3A5, HSPG2, |
| | | | CCL3, MYD88, VIP, SOAT1, |
| | | | ADRBK1, NR4A2, MMP8, NPR2, |
| | | | GCH1, EPRS, PPARGC1A, F12, |
| | | | PECAM1, CCL4, CERPINA34, |
| | | | CASR, FABP2, TTF2, PROS1, CTF1, |
| | | | SGCB, YME1L1, CAMP, ZC3H12A, |
| | | | AKR1B1, MMP7, AHR, CSF1, |
| | | | HDAC9, CTGF, KCNMA1, UGT1A, |
| | | | PRKCA, COMT, S100B, EGR1, PRL, |
| | | | IL15, DRD4, CAMK2G, SLC22A2, |
| | | | CCL11, PGF, THPO, GP6, TACR1, |
| | | | NTS, HNF1A, SST, KCDN1, |
| | | | LOC646627, TBXAS1, CUP2J2, |
| | | | TBXA2R, ADH1C, ALOX12, AHSG, |
| | | | BHMT, GJA4, SLC25A4, ACLY, |
| | | | ALOX5AP, NUMA1, CYP27B1, |
| | | | CYSLTR2, SOD3, LTC4S, UCN, |
| | | | GHRL, APOC2, CLEC4A, |
| | | | KBTBD10, TNC, TYMS, SHC1, |
| | | | LRP1, SOCS3, ADHIB, KLK3, |
| | | | HSD11B1, VKORC1, SERPINB2, |
| | | | TNS1, RNF19A, EPOR, ITGAM, |
| | | | PITX2, MAPK7, FCGR3A, LEEPR, |
| | | | ENG, GPX1, GOT2, HRH1, NR112, |
| | | | CRH, HTR1A, VDACI, HPSE, |
| | | | SFTPD, TAP2, RMF123, PTK2Bm |
| | | | NTRK2, IL6R, ACHE, GLP1R, GHR, |
| | | | GSR, NQO1, NR5A1, GJB2, |
| | | | SLC9A1, MAOA, PCSK9, FCGR2A, |
| | | | SERPINF1, EDN3, UCP2, TFAP2A, |
| | | | C4BPA, SERPINF2, TYMP, ALPP, |
| | | | CXCR2, SLC3A3, ABCG2, ADA, |
| | | | JAK3, HSPA1A, FASN, FGF1, F11, |
| | | | ATP7A, CR1, GFPA, ROCK1, |
| | | | MECP2, MYLK, BCHE, LIPE, |
| | | | ADORA1, WRN, CXCR3, CD81, |
| | | | SMAD7, LAMC2, MAP3K5, CHGA, |
| | | | IAPP, RHO, ENPP1, PTHLH, NRG1, |
| | | | VEGFC, ENPEP, CEBPB, NAGLU,. |
| | | | F2RL3, CX3CL1, BDKRB1, |
| | | | ADAMTS13, ELANE, ENPP2, CISH, |
| | | | GAST, MYOC, ATP1A2, NF1, GJB1, |
| | | | MEF2A, VCL, BMPR2, TUBB, |
| | | | CDC42, KRT18, HSF1, MYB, |
| | | | PRKAA2, ROCK2, TFP1, PRKG1, |
| | | | BMP2, CTNND1, CTH, CTSS, |
| | | | VAV2, NPY2R, IGFBP2, CD28, |
| | | | GSTA1, PPIA, APOH, S100A8, IL11, |
| | | | ALOX15, FBLN1, NR1H3, SCD, GIP, |
| | | | CHGB, PRKCB, SRD5A1, HSD11B2, |
| | | | CALCRL, GALNT2, ANGPTL4, |
| | | | KCNN4, PIK3C2A, HBEGF, |
| | | | CYP7A1, HLA-DRB5, BNIP3, |
| | | | GCKR, S100A12, PADI4, HSPA14, |
| | | | CXCR1, H19, KRTAP19-3, IDDM2, |
| | | | RAC2, YRY1, CLOCK, NGFR, DBH, |
| | | | CRNA COMPONENT4, CACNA1C, |
| | | | PRKAG2, CHAT, PTGDS, NR1H2, |
| | | | TEK, VEGFB, MEF2C, MAPKAPK2, |
| | | | TNFRSF11A, HSPA9, CYSLTR1, |
| | | | MATIA, OPRL1, IMPA1, CLCN2, |
| | | | DLD, PSMA6, PSMB8, CHI3L1, |
| | | | ALDH1B1, PARP2, STAR, LBP, |
| | | | ABCC6, RGS2, EFNB2, GJB6, |
| | | | APOA2, AMPD1, DYSF, |
| | | | FDFT1,EMD2, CCR6, GJB3, ILIRL1, |
| | | | ENTPD1, BBS4, CELSR2, F11R, |
| | | | RAPGEF3, HYAL1, ZNF259, |
| | | | ATOX1, ATF6, KHK, SAT1, GGH, |
| | | | TIMP4, SLC4A4, PDE2A, PDE3B, |
| | | | FADS1, FADS2, TMSB4X, TXNIP, |
| | | | LIMS1, RHOB, LY96, FOXO1, |
| | | | PNPLA2, TRH, GJC1, S: C17A5, FTO, |
| | | | GJD2, PRSC1, CASP12, GPBAR1, |
| | | | PXK, IL33, TRIB1, PBX4, NUPR1, |
| | | | 15-SEP, CILP2, TERC, GGT2, |
| | | | MTCO1, UOX, AVP |
| Cataract | eye | | CRYAA, CRYA1, CRYBB2, CRYB2, |
| | | | PITX3, BFSP2, CP49, CP47, CRYAA, |
| | | | CRYA1, PAX6, AN2, MGDA, |
| | | | CRYBA1, CRYB1, CRYGC, CRYG3, |
| | | | CCL, LIM2, MP19, CRYGD, CRYG4, |
| | | | BFSP2, CP49, CP47, HSF4, CTM, |
| | | | HSF4, CTM, MIP, AQPO, CRYAB, |
| | | | CRYA2, CTPP2, CRYBB1, CRYGD, |
| | | | CRYG4, CRYBB2, CRYB2, CRYGC, |
| | | | CRYG3, CCL, CRYAA, CRYA1, |
| | | | GJA8, CX50, CAE1, GJA3, CX46, |
| | | | CZP3, CAE3, CCM1, CAM, KRIT1 |
| CDKL-5 Deficiencies or | Brain, CNS | | CDKL5 |
| Mediated Diseases | | | |
| Charcot-Marie-Tooth (CMT) | Nervous system | Muscles | PMP22 (CMT1A and E), MPZ |
| disease (Types 1, 2, 3, 4,) | | (dystrophy) | (CMT1B), LITAF (CMT1C), EGR2 |
| | | | (CMT1D), NEFL (CMT1F), GJB1 |
| | | | (CMT1X), MFN2 (CMT2A), KIF1B |
| | | | (CMT2A2B), RAB7A (CMT2B), |
| | | | TRPV4 (CMT2C), GARS (CMT2D), |
| | | | NEFL (CMT2E), GAPD1 (CMT2K), |
| | | | HSPB8 (CMT2L), DYNC1H1, |
| | | | CMT20), LRSAMI (CMT2P), |
| | | | IGHMBP2 (CMT2S), MORC2 |
| | | | (CMT2Z), GDAP1 (CMT4A), |
| | | | MTMR2 or SBF2/MTMR13 |
| | | | (CMT4B), SH3TC2 (CMT4C), |
| | | | NDRG1 (CMT4D), PRX (CMT4F), |
| | | | FIG4 (CMT4J), NT-3 |
| Chédiak-Higashi Syndrome | Immune system | Skin, hair, eyes, | LYST |
| | | neurons | |
| Choroidermia | | | CHM, REP1, |
| Chorioretinal atrophy | eye | | PRDM13, RGR, TEAD1 |
| Chronic Granulomatous Disease | Immune system | | CYBA, CYBB, NCF1, NCF2, NCF4 |
| Chronic Mucocutaneous | Immune system | | AIRE, CARD9, CLEC7A IL12B, |
| Candidiasis | | | IL12B1, IL1F, IL17RA, IL17RC, |
| | | | RORC, STAT1, STAT3, TRAF31P2 |
| Cirrhosis | liver | | KRT18, KRT8, CIRH1A, NAIC, |
| | | | TEX292, KIAA1988 |
| Colon cancer (Familial | Gastrointestinal | | FAP: APC HNPCC: MSH2, |
| adenomatous polyposis (FAP) | | | MLH1, PMS2, SH6, PMS1 |
| and hereditary nonpolyposis | | | |
| colon cancer (HNPCC)) | | | |
| Combined Immunodeficiency | Immune System | | IL2RG, SCIDX1, SCIDX, IMD4); |
| | | | HIV-1 (CCL5, SCYA5, D17S136E, |
| | | | TCP228 |
| Cone(-rod) dystrophy | eye | | AIPL1, CRX, GUA1A, GUCY2D, |
| | | | PITPM3, PROM1, PRPH2, RIMS1, |
| | | | SEMA4A, ABCA4, ADAM9, ATF6, |
| | | | C21ORF2, C8ORF37, CACNA2D4, |
| | | | CDHR1, CERKL, CNGA3, CNGB3, |
| | | | CNNM4, CNAT2, IFT81, KCNV2, |
| | | | PDE6C, PDE6H, POCIB, RAX2, |
| | | | RDH5, RPGRIP1, TTLL5, RetCG1, |
| | | | GUCY2E |
| Congenital Stationary Night | eye | | CABP4, CACNA1F, CACNA2D4, |
| Blindness | | | GNAT1, CPR179, GRK1, GRM6, |
| | | | LRIT3, NYX, PDE6B, RDH5, RHO, |
| | | | RLBP1, RPE65, SAG, SLC24A1, |
| | | | TRPM1, |
| Congenital Fructose Intolerance | Metabolism | | ALDOB |
| Cori's Disease (Glycogen | Various- | | AGL |
| Storage Disease Type III) | wherever | | |
| | glycogen | | |
| | accumulates, | | |
| | particularly | | |
| | liver, heart, | | |
| | skeletal muscle | | |
| Corneal clouding and dystrophy | eye | | APOA1, TGFBI, CSD2, CDGG1, |
| | | | CSD, BIGH3, CDG2, TACSTD2, |
| | | | TROP2, MIS1, VSX1, RINX, PPCD, |
| | | | PPD, KTCN, COL8A2, FECD, |
| | | | PPCD2, PIP5K3, CFD |
| Cornea plana congenital | | | KERA, 12 |
| Cri du chat Syndrome, also | | | Deletions involving only band 5p15.2 |
| known as 5p syndrome and cat | | | to the entire short arm of chromosome |
| cry syndrome | | | 5, e.g., CTNND2, TERT, |
| Cystic Fibrosis (CF) | Lungs and | Pancreas, liver, | CTFR, ABCC7, CF, MRP7, SCNN1A, |
| | respiratory | digestive | those described in WO2015157070 |
| | system | system, | |
| | | reproductive | |
| | | system, | |
| | | exocrine, glands, | |
| Diabetic nephropathy | kidney | | Gremlin, 12/15- lipoxygenase, TIM44, |
| Dent Disease (Types 1 and 2) | Kidney | | Type 1: CLCN5, Type 2: ORCL |
| Dentatorubro-Pallidoluysian | CNS, brain, | | Atrophin-1 and Atn1 |
| Atrophy (DRPLA) (aka Haw | muscle | | |
| River and Naito-Oyanagi | | | |
| Disease) | | | |
| Down Syndrome | various | | Chromosome 21 trisomy |
| Drug Addiction | Brain | | Prkce; Drd2; Drd4; ABAT; |
| | | | GRIA2; Grm5; Grin1; Htr1b; Grin2a; |
| | | | Drd3; Pdyn; Gria1 |
| Duane syndrome (Types 1, 2, and | eye | | CHN1, indels on chromosomes 4 and 8 |
| 3, including subgroups A, B and | | | |
| C). Other names for this | | | |
| condition include: Duane's | | | |
| Retraction Syndrome (or DR | | | |
| syndrome), Eye Retraction | | | |
| Syndrome, Retraction Syndrome, | | | |
| Congenital retraction syndrome | | | |
| and Stilling-Turk-Duane | | | |
| Syndrome | | | |
| Duchenne muscular dystrophy | muscle | Cardiovascular, | DMD, BMD, dystrophin gene, intron |
| (DMD) | | respiratory | flanking exon 51 of DMD gene, exon |
| | | | 51 mutations in DMD gene, see also |
| | | | WO2013163628 and US Pat. Pub. |
| | | | 20130145487 |
| Edward's Syndrome | | | Complete or partial trisomy of |
| (Trisomy 18) | | | chromosome 18 |
| Ehlers-Danlos Syndrome (Types | Various | | COL5A1, COL5A2, COL1A1, |
| I-VI) | depending on | | COL3A1, TNXB, PLOD1, COL1A2, |
| | type: including | | FKBP14 and ADAMTS2 |
| | musculoskeletal, | | |
| | eye, vasculature, | | |
| | immune, and | | |
| | skin | | |
| Emery-Dreifuss muscular | muscle | | LMNA, LMN1, EMD2, FPLD, |
| dystrophy | | | CMD1A, HGPS, LGMD1B, LMNA, |
| | | | LMN1, EMD2, FPLD, CMD1A |
| Enhanced S-Cone Syndrome | eye | | NR2E3, NRL |
| Fabry's Disease | Various - | | GLA |
| | including skin, | | |
| | eyes, and | | |
| | gastrointestinal | | |
| | system, kidney, | | |
| | heart, brain, | | |
| | nervous system | | |
| Facioscapulohumeral muscular | muscles | | FSHMD1A, FSHD1A, FRG1, |
| dystrophy | | | |
| Factor H and Factor H-like 1 | blood | | HF1, CFH, HUS |
| Factor V Leiden thrombophilia | blood | | Factor V (F5) |
| and Factor V deficiency | | | |
| Factor V and Factor VII | blood | | MCFD2 |
| deficiency | | | |
| Factor VII deficiency | blood | | F7 |
| Factor X deficiency | blood | | F10 |
| Factor XI deficiency | blood | | F11 |
| Factor XII deficiency | blood | | F12, HAF |
| Factor XIIIA deficiency | blood | | F13A1, F13A |
| Factor XIIIB deficiency | blood | | F13B |
| Familial Hypercholestereolemia | Cardiovascular | | APOB, LDLR, PCSK9 |
| | system | | |
| Familial Mediterranean Fever | Various- | Heart, kidney, | MEFV |
| (FMF) also called recurrent | organs/tissues | brain/CNS, | |
| polyserositis or familial | with serous or | reproductive | |
| paroxysmal polyserositis | synovial | organs | |
| | membranes, | | |
| | skin, joints | | |
| Fanconi Anemia | Various - blood | | FANCA, FACA, FA1, FA, FAA, |
| | (anemia), | | FAAP95, FAAP90, FLJ34064, |
| | immune system, | | FANCC, FANCG, RAD51, BRCA1, |
| | cognitive, | | BRCA2, BRIP1, BACH1, FANCJ, |
| | kidneys, eyes, | | FANCB, FANCD1, FANCD2, |
| | musculoskeletal | | FANCD, FAD, FANCE, FACE, |
| | | | FANCF, FANCI, ERCC4, FANCL, |
| | | | FANCM, PALB2, RAD51C, SLX4, |
| | | | UBE2T, FANCB, XRCC9, PHF9, |
| | | | KIAA1596 |
| Fanconi Syndrome Types I | kidneys | | FRTS1, GATM |
| (Childhood onset) and II (Adult | | | |
| Onset) | | | |
| Fragile X syndrome and related | brain | | FMR1, FMR2; FXR1; FXR2; |
| disorders | | | mGLUR5 |
| Fragile XE Mental Retardation | Brain, nervous | | FMR1 |
| (aka Martin Bell syndrome) | system | | |
| Friedreich Ataxia (FRDA) | Brain, nervous | heart | FXN/X25 |
| | system | | |
| Fuchs endothelial corneal | Eye | | TCF4; COL8A2 |
| dystrophy | | | |
| Galactosemia | Carbohydrate | Various-where | GALT, GALK1, and GALE |
| | metabolism | galactose | |
| | disorder | accumulates - | |
| | | liver, brain, eyes | |
| Gastrointestinal Epithelial | | | CISH |
| Cancer, GI cancer | | | |
| Gaucher Disease (Types 1, 2, and | Fat metabolism | Various-liver, | GBA |
| 3, as well as other unusual forms | disorder | spleen, blood, | |
| that may not fit into these types) | | CNS, skeletal | |
| | | system | |
| Griscelli syndrome | | | |
| Glaucoma | eye | | MYOC, TIGR, GLC1A, JOAG, |
| | | | GPOA, OPTN, GLC1E, FIP2, HYPL, |
| | | | NRP, CYP1B1, GLC3A, OPA1, NTG, |
| | | | NPG, CYP1B1, GLC3A, those |
| | | | described in WO2015153780 |
| Glomerulo sclerosis | kidney | | CC chemokine ligand 2 |
| Glycogen Storage Diseases | Metabolism | | SLC2A2, GLUT2, G6PC, G6PT, |
| Types I-VI -See also Cori's | Diseases | | G6PT1, GAA, LAMP2, LAMPB, |
| Disease, Pompe's Disease, | | | AGL, GDE, GBE1, GYS2, PYGL, |
| McArdle's disease, Hers Disease, | | | PFKM, see also Cori's Disease, |
| and Von Gierke's disease | | | Pompe's Disease, McArdle's disease, |
| | | | Hers Disease, and Von Gierke's |
| | | | disease |
| RBC Glycolytic enzyme | blood | | any mutations in a gene for an enzyme |
| deficiency | | | in the glycolysis pathway including |
| | | | mutations in genes for hexokinases I |
| | | | and II, glucokinase, phosphoglucose |
| | | | isomerase, phosphofructokinase, |
| | | | aldolase Bm triosephosphate |
| | | | isomerease, glyceraldehydee-3- |
| | | | phosphate dehydrogenase, |
| | | | phosphoglycerokinase, |
| | | | phosphoglycerate mutase, enolase I, |
| | | | pyruvate kinase |
| Hartnup's disease | Malabsorption | Various- brain, | SLC6A19 |
| | disease | gastrointestinal, | |
| | | skin, | |
| Hearing Loss | ear | | NOX3, Hes5, BDNF, |
| Hemochromatosis (HH) | Iron absorption | Various- | HFE and H63D |
| | regulation | wherever iron | |
| | disease | accumulates, | |
| | | liver, heart, | |
| | | pancreas, joints, | |
| | | pituitary gland | |
| Hemophagocytic | blood | | PRF1, HPLH2, UNC13D, MUNC13- |
| lymphohistiocytosis disorders | | | 4, HPLH3, HLH3, FHL3 |
| Hemorrhagic disorders | blood | | PI, ATT, F5 |
| Hers disease (Glycogen storage | liver | muscle | PYGL |
| disease Type VI) | | | |
| Hereditary angioedema (HAE) | | | kalikrein B1 |
| Hereditary Hemorrhagic | Skin and | | ACVRL1, ENG and SMAD4 |
| Telangiectasia (Osler-Weber- | mucous | | |
| Rendu Syndrome) | membranes | | |
| Hereditary Spherocytosis | blood | | NK1, EPB42, SLC4A1, SPTA1, and |
| | | | SPTB |
| Hereditary Persistence of Fetal | blood | | HBG1, HBG2, BCL11A, promoter |
| Hemoglobin | | | region of HBG 1 and/or 2 (in the |
| | | | CCAAT box) |
| Hemophilia (hemophilia A | blood | | A: FVIII, F8C, HEMA |
| (Classic) a B (aka Christmas | | | B: FVIX, HEMB |
| disease) and C) | | | C: F9, F11 |
| Hepatic adenoma | liver | | TCF1, HNF1A, MODY3 |
| Hepatic failure, early onset, and | liver | | SCOD1, SCO1 |
| neurologic disorder | | | |
| Hepatic lipase deficiency | liver | | LIPC |
| Hepatoblastoma, cancer and | liver | | CTNNB1, PDGFRL, PDGRL, PRLTS, |
| carcinomas | | | AXIN1, AXIN, CTNNB1, TP53, P53, |
| | | | LFS1, IGF2R, MPRI, MET, CASP8, |
| | | | MCH5 |
| Hermansky-Pudlak syndrome | Skin, eyes, | | HPS1, HPS3, HPS4, HPS5, HPS6, |
| | blood, lung, | | HPS7, DTNBP1, BLOC1, BLOC1S2, |
| | kidneys, | | BLOC3 |
| | intestine | | |
| HIV susceptibility or infection | Immune system | | IL10, CSIF, CMKBR2, CCR2, |
| | | | CMKBR5, CCCKR5 (CCR5), those in |
| | | | WO2015148670A1 |
| Holoprosencephaly (HPE) | brain | | ACVRL1, ENG, SMAD4 |
| (Alobar, Semilobar, and Lobar) | | | |
| Homocystinuria | Metabolic | Various- | CBS, MTHFR, MTR, MTRR, and |
| | disease | connective | MMADHC |
| | | tissue, muscles, | |
| | | CNS, | |
| | | cardiovascular | |
| | | system | |
| HPV | | | HPV16 and HPV18 E6/E7 |
| HSV1, HSV2, and related | eye | | HSV1 genes (immediate early and late |
| keratitis | | | HSV-1 genes (UL1, 1.5, 5, 6, 8, 9, 12, |
| | | | 15, 16, 18, 19, 22, 23, 26, 26.5, 27, 28, |
| | | | 29, 30, 31, 32, 33, 34, 35, 36, 37, 38, |
| | | | 42, 48, 49.5, 50, 52, 54, S6, RL2, RS1, |
| | | | those described in WO2015153789, |
| | | | WO2015153791 |
| Hunter's Syndrome (aka | Lysosomal | Various- liver, | IDS |
| Mucopolysaccharidosis type II) | storage disease | spleen, eye, | |
| | | joint, heart, | |
| | | brain, skeletal | |
| Huntington's disease (HD) and | Brain, nervous | | HD, HTT, IT15, PRNP, PRIP, JPH3, |
| HD-like disorders | system | | JP3, HDL2, TBP, SCA17, PRKCE; |
| | | | IGF1; EP300; RCOR1; PRKCZ; |
| | | | HDAC4; and TGM2, and those |
| | | | described in WO2013130824, |
| | | | WO2015089354 |
| Hurler's Syndrome (aka | Lysosomal | Various- liver, | IDUA, α-L-iduronidase |
| mucopolysaccharidosis type I H, | storage disease | spleen, eye, | |
| MPS IH) | | joint, heart, | |
| | | brain, skeletal | |
| Hurler-Scheie syndrome (aka | Lysosomal | Various- liver, | IDUA, α-L-iduronidase |
| mucopolysaccharidosis type I H- | storage disease | spleen, eye, | |
| S, MPS I H-S) | | joint, heart, | |
| | | brain, skeletal | |
| hyaluronidase deficiency (aka | Soft and | | HYAL1 |
| MPS IX) | connective | | |
| | tissues | | |
| Hyper IgM syndrome | Immune system | | CD40L |
| Hyper- tension caused renal | kidney | | Mineral corticoid receptor |
| damage | | | |
| Immunodeficiencies | Immune System | | CD3E, CD3G, AICDA, AID, HIGM2, |
| | | | TNFRSF5, CD40, UNG, DGU, |
| | | | HIGM4, TNFSF5, CD40LG, HIGM1, |
| | | | IGM, FOXP3, IPEX, AIID, XPID, |
| | | | PIDX, TNFRSF14B, TACI |
| Inborn errors of metabolism: | Metabolism | Various organs | See also: Carbohydrate metabolism |
| including urea cycle disorders, | diseases, liver | and cells | disorders (e.g., galactosemia), Amino |
| organic acidemias), fatty acid | | | acid Metabolism disorders (e.g., |
| oxidation defects, amino | | | phenylketonuria), Fatty acid |
| acidopathies, carbohydrate | | | metabolism (e.g., MCAD deficiency), |
| disorders, mitochondrial | | | Urea Cycle disorders (e.g., |
| disorders | | | Citrullinemia), Organic acidemias |
| | | | (e.g., Maple Syrup Urine disease), |
| | | | Mitochondrial disorders (e.g., |
| | | | MELAS), peroxisomal disorders (e.g., |
| | | | Zellweger syndrome) |
| Inflammation | Various | | IL-10; IL-1 (IL-1a; IL-1b); IL-13; IL- |
| | | | 17 (IL-17a (CTLA8); IL- |
| | | | 17b; IL-17c; IL-17d; IL-17f); II-23; |
| | | | Cx3cr1; ptpn22; TNFa; |
| | | | NOD2/CARD15 for IBD; IL-6; IL-12 |
| | | | (IL-12a; IL-12b); |
| | | | CTLA4; Cx3cl1 |
| Inflammatory Bowel Diseases | Gastrointestinal | Joints, skin | NOD2, IRGM, LRRK2, ATG5, |
| (e.g., Ulcerative Colitis and | | | ATG16L1, IRGM, GATM, ECM1, |
| Chron's Disease) | | | CDH1, LAMB1, HNF4A, GNA12, |
| | | | IL10, CARD9/15. CCR6, IL2RA, |
| | | | MST1, TNFSF15, REL, STAT3, |
| | | | IL23R, IL12B, FUT2 |
| Interstitial renal fibrosis | kidney | | TGF-β type II receptor |
| Job's Syndrome (aka Hyper IgE | Immune System | | STAT3, DOCK8 |
| Syndrome) | | | |
| Juvenile Retinoschisis | eye | | RS1, XLRS1 |
| Kabuki Syndrome 1 | | | MLL4, KMT2D |
| Kennedy Disease (aka | Muscles, brain, | | SBMA/SMAX1/AR |
| Spinobulbar Muscular Atrophy) | nervous system | | |
| Klinefelter syndrome | Various- | | Extra X chromosome in males |
| | particularly | | |
| | those involved | | |
| | in development | | |
| | of male | | |
| | characteristics | | |
| Lafora Disease | Brain, CNS | | EMP2A and EMP2B |
| Leber Congenital Amaurosis | eye | | CRB1, RP12, CORD2, CRD, CRX, |
| | | | IMPDH1, OTX2, AIPL1, CABP4, |
| | | | CCT2, CEP290, CLUAP1, CRB1, |
| | | | CRX, DTHD1, GDF6, GUCY2D, |
| | | | IFT140, IQCB1, KCNJ13, LCA5, |
| | | | LRAT, NMNAT1, PRPH2, RD3, |
| | | | RDH12, RPE65, RP20, RPGRIP1, |
| | | | SPATA7, TULP1, LCA1, LCA4, |
| | | | GUC2D, CORD6, LCA3, |
| Lesch-Nyhan Syndrome | Metabolism | Various - joints, | HPRT1 |
| | disease | cognitive, brain, | |
| | | nervous system | |
| Leukocyte deficiencies and | blood | | ITGB2, CD18, LCAMB, LAD, |
| disorders | | | EIF2B1, EIF2BA, EIF2B2, EIF2B3, |
| | | | EIF2B5, LVWM, CACH, CLE, |
| | | | EIF2B4 |
| Leukemia | Blood | | TAL1, TCL5, SCL, TAL2, FLT3, |
| | | | NBS1, NBS, ZNFN1A1, IK1, LYF1, |
| | | | HOXD4, HOX4B, BCR, CML, PHL, |
| | | | ALL, ARNT, KRAS2, RASK2, |
| | | | GMPS, AF10, ARHGEF12, LARG, |
| | | | KIAA0382, CALM, CLTH, CEBPA, |
| | | | CEBP, CHIC2, BTL, FLT3, KIT, |
| | | | PBT, LPP, NPM1, NUP214, D9S46E, |
| | | | CAN, CAIN, RUNX1, CBFA2, |
| | | | AML1, WHSC1L1, NSD3, FLT3, |
| | | | AF1Q, NPM1, NUMA1, ZNF145, |
| | | | PLZF, PML, MYL, STAT5B, AF10, |
| | | | CALM, CLTH, ARL11, ARLTS1, |
| | | | P2RX7, P2X7, BCR, CML, PHL, |
| | | | ALL, GRAF, NF1, VRNF, WSS, |
| | | | NFNS, PTPN11, PTP2C, SHP2, NS1, |
| | | | BCL2, CCND1, PRAD1, BCL1, |
| | | | TCRA, GATA1, GF1, ERYF1, NFE1, |
| | | | ABL1, NQO1, DIA4, NMOR1, |
| | | | NUP214, D9S46E, CAN, CAIN |
| Limb-girdle muscular dystrophy | muscle | | LGMD |
| diseases | | | |
| Lowe syndrome | brain, eyes, | | OCRL |
| | kidneys | | |
| Lupus glomerulo- nephritis | kidney | | MAPK1 |
| Machado- | Brain, CNS, | | ATX3 |
| Joseph's Disease (also known as | muscle | | |
| Spinocerebellar ataxia Type 3) | | | |
| Macular degeneration | eye | | ABC4, CBC1, CHM1, APOE, |
| | | | C1QTNF5, C2, C3, CCL2, CCR2, |
| | | | CD36, CFB, CFH, CFHR1, CFHR3, |
| | | | CNGB3, CP, CRP, CST3, CTSD, |
| | | | CX3CR1, ELOVL4, ERCC6, FBLN5, |
| | | | FBLN6, FSCN2, HMCN1, HTRA1, |
| | | | IL6, IL8, PLEKHAI, PROM1, |
| | | | PRPH2, RPGR, SERPING1, TCOF1, |
| | | | TIMP3, TLR3 |
| Macular Dystrophy | eye | | BEST1, C1QTNF5, CTNNA1, |
| | | | EFEMP1, ELOVL4, FSCN2, |
| | | | GUCA1B, HMCN1, IMPG1, OTX2, |
| | | | PRDM13, PROMI, PRPH2, RPIL1, |
| | | | TIMP3, ABCA4, CFH, DRAM2, |
| | | | IMG1, MFSD8, ADMD, STGD2, |
| | | | STGD3, RDS, RP7, PRPH, AVMD, |
| | | | AOFMD, VMD2 |
| Malattia Leventinesse | eye | | EFEMP1, FBLN3 |
| Maple Syrup Urine Disease | Metabolism | | BCKDHA, BCKDHB, and DBT |
| | disease | | |
| Marfan syndrome | Connective | Musculoskeletal | FBN1 |
| | tissue | | |
| Maroteaux-Lamy Syndrome (aka | Musculoskeletal | Liver, spleen | ARSB |
| MPS VI) | system, nervous | | |
| | system | | |
| McArdle's Disease (Glycogen | Glycogen | muscle | PYGM |
| Storage Disease Type V) | storage disease | | |
| Medullary cystic kidney disease | kidney | | UMOD, HNFJ, FJHN, MCKD2, |
| | | | ADMCKD2 |
| Metachromatic leukodystrophy | Lysosomal | Nervous system | ARSA |
| | storage disease | | |
| Methylmalonic acidemia (MMA) | Metabolism | | MMAA, MMAB, MUT, MMACHC, |
| | disease | | MMADHC, LMBRD1 |
| Morquio Syndrome (aka MPS IV | Connective | heart | GALNS |
| A and B) | tissue, skin, | | |
| | bone, eyes | | |
| Mucopolysaccharidosis diseases | Lysosomal | | See also Hurler/Scheie syndrome, |
| (Types I H/S, I H, II, III A B and | storage disease - | | Hurler disease, Sanfillipo syndrome, |
| C, I S, IVA and B, IX, VII, and | affects various | | Scheie syndrome, Morquio syndrome, |
| VI) | organs/tissues | | hyaluronidase deficiency, Sly |
| | | | syndrome, and Maroteaux-Lamy |
| | | | syndrome |
| Muscular Atrophy | muscle | | VAPB, VAPC, ALS8, SMN1, SMA1, |
| | | | SMA2, SMA3, SMA4, BSCL2, |
| | | | SPG17, GARS, SMAD1, CMT2D, |
| | | | HEXB, IGHMBP2, SMUBP2, |
| | | | CATF1, SMARD1 |
| Muscular dystrophy | muscle | | FKRP, MDC1C, LGMD2I, LAMA2, |
| | | | LAMM, LARGE, KIAA0609, |
| | | | MDC1D, FCMD, TTID, MYOT, |
| | | | CAPN3, CANP3, DYSF, LGMD2B, |
| | | | SGCG, LGMD2C, DMDA1, SCG3, |
| | | | SGCA, ADL, DAG2, LGMD2D, |
| | | | DMDA2, SGCB, LGMD2E, SGCD, |
| | | | SGD, LGMD2F, CMD1L, TCAP, |
| | | | LGMD2G, CMD1N, TRIM32, HT2A, |
| | | | LGMD2H, FKRP, MDCIC, LGMD2I, |
| | | | TTN, CMD1G, TMD, LGMD2J, |
| | | | POMT1, CAV3, LGMDIC, SEPN1, |
| | | | SELN, RSMD1, PLECI, PLTN, EBS1 |
| Myotonic dystrophy (Type 1 and | Muscles | Eyes, heart, | CNBP (Type 2) and DMPK (Type 1) |
| Type 2) | | endocrine | |
| Neoplasia | | | PTEN; ATM; ATR; EGFR; ERBB2; |
| | | | ERBB3; ERBB4; |
| | | | Notch1; Notch2; Notch3; Notch4; |
| | | | AKT; AKT2; AKT3; HIF; |
| | | | HIF1a; HIF3a; Met; HRG; Bcl2; |
| | | | PPAR alpha; PPAR |
| | | | gamma; WT1 (Wilms Tumor); FGF |
| | | | Receptor Family |
| | | | members (5 members: 1, 2, 3, 4, 5); |
| | | | CDKN2a; APC; RB |
| | | | (retinoblastoma); MEN1; VHL; |
| | | | BRCA1; BRCA2; AR |
| | | | (Androgen Receptor); TSG101; IGF; |
| | | | IGF Receptor; Igf1 (4 |
| | | | variants); Igf2 (3 variants); Igf 1 |
| | | | Receptor, Igf 2 Receptor; |
| | | | Bax; Bcl2; caspases family (9 |
| | | | members: |
| | | | 1, 2, 3, 4, 6, 7, 8, 9, 12); Kras; Apc |
| Neurofibromatosis (NF) (NF1, | brain, spinal | | NF1, NF2 |
| formerly Recklinghausen's NF, | cord, nerves, | | |
| and NF2) | and skin | | |
| Niemann-Pick Lipidosis (Types | Lysosomal | Various- where | Types A and B: SMPD1; Type C: |
| A, B, and C) | Storage Disease | sphingomyelin | NPC1 or NPC2 |
| | | accumulates, | |
| | | particularly | |
| | | spleen, liver, | |
| | | blood, CNS | |
| Noonan Syndrome | Various - | | PTPN11, SOS1, RAF1 and KRAS |
| | musculoskeletal, | | |
| | heart, eyes, | | |
| | reproductive | | |
| | organs, blood | | |
| Norrie Disease or X-linked | eye | | NDP |
| Familial Exudative | | | |
| Vitreoretinopathy | | | |
| North Carolina Macular | eye | | MCDR1 |
| Dystrophy | | | |
| Osteogenesis imperfecta (OI) | bones, | | COL1A1, COL1A2, CRTAP, P3H |
| (Types I, II, III, IV, V, VI, VII) | musculoskeletal | | |
| Osteopetrosis | bones | | LRP5, BMND1, LRP7, LR3, OPPG, |
| | | | VBCH2, CLCN7, CLC7, OPTA2, |
| | | | OSTM1, GL, TCIRG1, TIRC7, |
| | | | OC116, OPTB1 |
| Patau's Syndrome | Brain, heart, | | Additional copy of chromosome 13 |
| (Trisomy 13) | skeletal system | | |
| Parkinson's disease (PD) | Brain, nervous | | SNCA (PARK1), UCHL1 (PARK 5), |
| | system | | and LRRK2 (PARK8), (PARK3), |
| | | | PARK2, PARK4, PARK7 (PARK7), |
| | | | PINK1 (PARK6); x-Synuclein, DJ-1, |
| | | | Parkin, NR4A2, NURR1, NOT, |
| | | | TINUR, SNCAIP, TBP, SCA17, |
| | | | NCAP, PRKN, PDJ, DBH, NDUFV2 |
| Pattern Dystrophy of the RPE | eye | | RDS/peripherin |
| Phenylketonuria (PKU) | Metabolism | Various due to | PAH, PKU1, QDPR, DHPR, PTS |
| | disorder | build-up of | |
| | | phenylalanine, | |
| | | phenyl ketones | |
| | | in tissues and | |
| | | CNS | |
| Polycystic kidney and hepatic | Kidney, liver | | FCYT, PKHD1, ARPKD, PKD1, |
| disease | | | PKD2, PKD4, PKDTS, PRKCSH, |
| | | | G19P1, PCLD, SEC63 |
| Pompe's Disease | Glycogen | Various - heart, | GAA |
| | storage disease | liver, spleen | |
| Porphyria (actually refers to a | Various- | | ALAD, ALAS2, CPOX, FECH, |
| group of different diseases all | wherever heme | | HMBS, PPOX, UROD, or UROS |
| having a specific heme | precursors | | |
| production process abnormality) | accumulate | | |
| posterior polymorphous corneal | eyes | | TCF4; COL8A2 |
| dystrophy | | | |
| Primary Hyperoxaluria (e.g., type | Various - eyes, | | LDHA (lactate dehydrogenase A) and |
| 1) | heart, kidneys, | | hydroxyacid oxidase 1 (HAO1) |
| | skeletal system | | |
| Primary Open Angle Glaucoma | eyes | | MYOC |
| (POAG) | | | |
| Primary sclerosing cholangitis | Liver, | | TCF4; COL8A2 |
| | gallbladder | | |
| Progeria (also called Hutchinson- | All | | LMNA |
| Gilford progeria syndrome) | | | |
| Prader-Willi Syndrome | Musculoskeletal | | Deletion of region of short arm of |
| | system, brain, | | chromosome 15, including UBE3A |
| | reproductive | | |
| | and endocrine | | |
| | system | | |
| Prostate Cancer | prostate | | HOXB13, MSMB, GPRC6A, TP53 |
| Pyruvate Dehydrogenase | Brain, nervous | | PDHA1 |
| Deficiency | system | | |
| Kidney/Renal carcinoma | kidney | | RLIP76, VEGF |
| Rett Syndrome | Brain | | MECP2, RTT, PPMX, MRX16, |
| | | | MRX79, CDKL5, STK9, MECP2, |
| | | | RTT, PPMX, MRX16, MRX79, x- |
| | | | Synuclein, DJ-1 |
| Retinitis pigmentosa (RP) | eye | | ADIPOR1, ABCA4, AGBL5, |
| | | | ARHGEF18, ARL2BP, ARL3, ARL6, |
| | | | BEST1, BBS1, BBS2, C2ORF71, |
| | | | C8ORF37, CA4, CERKL, CLRN1, |
| | | | CNGA1, CMGB1, CRB1, CRX, |
| | | | CYP4V2, DHDDS, DHX38, EMC1, |
| | | | EYS, FAM161A, FSCN2, GPR125, |
| | | | GUCA1B, HK1, HPRPF3, HGSNAT, |
| | | | IDH3B, IMPDH1, IMPG2, IFT140, |
| | | | IFT172, KLHL7, KIAA1549, KIZ, |
| | | | LRAT, MAK, MERTK, MVK, NEK2, |
| | | | NUROD1, NR2E3, NRL, OFD1, |
| | | | PDE6A, PDE6B, PDE6G, POMGNT1, |
| | | | PRCD, PROM1, PRPF3, PRPF4, |
| | | | PRPF6, PRPF8, PRPF31, PRPH2, |
| | | | RPB3, RDH12, REEP6, RP39, RGR, |
| | | | RHO, RLBP1, ROMI, RP1, RP1L1, |
| | | | RPY, RP2, RP9, RPE65, RPGR, |
| | | | SAMD11, SAG, SEMA4A, SLC7A14, |
| | | | SNRNP200, SPP2, SPATA7, TRNT1, |
| | | | TOPORS, TTC8, TULP1, USH2A, |
| | | | ZFN408, ZNF513, see also |
| | | | 20120204282 |
| Scheie syndrome (also known as | Various- liver, | | IDUA, α-L-iduronidase |
| mucopolysaccharidosis type I | spleen, eye, | | |
| S(MPS I-S)) | joint, heart, | | |
| | brain, skeletal | | |
| Schizophrenia | Brain | | Neuregulin1 (Nrg1); Erb4 (receptor for |
| | | | Neuregulin); |
| | | | Complexin1 (Cplx1); Tph1 |
| | | | Tryptophan hydroxylase; Tph2 |
| | | | Tryptophan hydroxylase 2; Neurexin |
| | | | 1; GSK3; GSK3a; |
| | | | GSK3b; 5-HTT (Slc6a4); COMT; |
| | | | DRD (Drdla); SLC6A3; DAOA; |
| | | | DTNBP1; Dao (Dao1); TCF4; |
| | | | COL8A2 |
| Secretase Related Disorders | Various | | APH-1 (alpha and beta); PSEN1; |
| | | | NCSTN; PEN-2; Nos1, Parp1, Nat1, |
| | | | Nat2, CTSB, APP, APH1B, PSEN2, |
| | | | PSENEN, BACE1, ITM2B, CTSD, |
| | | | NOTCH1, TNF, INS, DYT10, |
| | | | ADAM17, APOE, ACE, STN, TP53, |
| | | | IL6, NGFR, IL1B, ACHE, CTNNB1, |
| | | | IGF1, IFNG, NRG1, CASP3, MAPK1, |
| | | | CDH1, APBB1, HMGCR, CREB1, |
| | | | PTGS2, HES1, CAT, TGFB1, ENO2, |
| | | | ERBB4, TRAPPC10, MAOB, NGF, |
| | | | MMP12, JAG1, CD40LG, PPARG, |
| | | | FGF2, LRP1, NOTCH4, MAPK8, |
| | | | PREP, NOTCH3, PRNP, CTSG, EGF, |
| | | | REN, CD44, SELP, GHR, ADCYAP1, |
| | | | INSR, GFAP, MMP3, MAPK10, SP1, |
| | | | MYC, CTSE, PPARA, JUN, TIMP1, |
| | | | IL5, IL1A, MMP9, HTR4, HSPG2, |
| | | | KRAS, CYCS, SMG1, IL1R1, |
| | | | PROK1, MAPK3, NTRK1, IL13, |
| | | | MME, TKT, CXCR2, CHRM1, |
| | | | ATXN1, PAWR, NOTCJ2, M6PR, |
| | | | CYP46A1, CSNK1D, MAPK14, |
| | | | PRG2, PRKCA, L1 CAM, CD40, |
| | | | NR1I2, JAG2, CTNND1, CMA1, |
| | | | SORT1, DLK1, THEM4, JUP, CD46, |
| | | | CCL11, CAV3, RNASE3, HSPA8, |
| | | | CASP9, CYP3A4, CCR3, TFAP2A, |
| | | | SCP2, CDK4, JOF1A, TCF7L2, |
| | | | B3GALTL, MDM2, RELA, CASP7, |
| | | | IDE, FANP4, CASK, ADCYAP1R1, |
| | | | ATF4, PDGFA, C21ORF33, SCG5, |
| | | | RMF123, NKFB1, ERBB2, CAV1, |
| | | | MMP7, TGFA, RXRA, STX1A, |
| | | | PSMC4, P2RY2, TNFRSF21, DLG1, |
| | | | NUMBL, SPN, PLSCR1, UBQLN2, |
| | | | UBQLN1, PCSK7, SPON1, SILV, |
| | | | QPCT, HESS, GCC1 |
| Selective IgA Deficiency | Immune system | | Type 1: MSH5; Type 2: TNFRSF13B |
| Severe Combined | Immune system | | JAK3, JAKL, DCLREIC, ARTEMIS, |
| Immunodeficiency (SCID) and | | | SCIDA, RAG1, RAG2, ADA, PTPRC, |
| SCID-X1, and ADA-SCID | | | CD45, LCA, IL7R, CD3D, T3D, |
| | | | IL2RG, SCIDX1, SCIDX, IMD4, |
| | | | those identified in US Pat. App. Pub. |
| | | | 20110225664, 20110091441, |
| | | | 20100229252, 20090271881 and |
| | | | 20090222937; |
| Sickle cell disease | blood | | HBB, BCL11A, BCL11Ae, cis- |
| | | | regulatory elements of the B-globin |
| | | | locus, HBG 1/2 promoter, HBG distal |
| | | | CCAAT box region between −92 and −130 |
| | | | of the HBG Transcription Start |
| | | | Site, those described in |
| | | | WO2015148863, WO 2013/126794, |
| | | | US Pat. Pub. 20110182867 |
| Sly Syndrome (aka MPS VII) | | | GUSB |
| Spinocerebellar Ataxias (SCA | | | ATXN1, ATXN2, ATX3 |
| types 1, 2, 3, 6, 7, 8, 12 and 17) | | | |
| Sorsby Fundus Dystrophy | eye | | TIMP3 |
| Stargardt disease | eye | | ABCR, ELOVL4, ABCA4, PROM1 |
| Tay-Sachs Disease | Lysosomal | Various - CNS, | HEX-A |
| | Storage disease | brain, eye | |
| Thalassemia (Alpha, Beta, Delta) | blood | | HBA1, HBA2 (Alpha), HBB (Beta), |
| | | | HBB and HBD (delta), LCRB, |
| | | | BCL11A, BCL11Ae, cis-regulatory |
| | | | elements of the B-globin locus, HBG |
| | | | 1/2 promoter, those described in |
| | | | WO2015148860, US Pat. Pub. |
| | | | 20110182867, 2015/148860 |
| Thymic Aplasia (DiGeorge | Immune system, | | deletion of 30 to 40 genes in the |
| Syndrome; 22q11.2 deletion | thymus | | middle of chromosome 22 at a location |
| syndrome) | | | known as 22q11.2, including TBX1, |
| | | | DGCR8 |
| Transthyretin amyloidosis | liver | | TTR (transthyretin) |
| (ATTR) | | | |
| trimethylaminuria | Metabolism | | FMO3 |
| | disease | | |
| Trinucleotide Repeat Disorders | Various | | HTT; SBMA/SMAX1/AR; |
| (generally) | | | FXN/X25 ATX3; |
| | | | ATXN1; ATXN2; |
| | | | DMPK; Atrophin-1 and Atn1 |
| | | | (DRPLA Dx); CBP (Creb-BP - global |
| | | | instability); VLDLR; Atxn7; Atxn10; |
| | | | FEN1, TNRC6A, PABPN1, JPH3, |
| | | | MED15, ATXNI, ATXN3, TBP, |
| | | | CACNAIA, ATXN80S, PPP2R2B, |
| | | | ATXN7, TNRC6B, TNRC6C, CELF3, |
| | | | MAB21L1, MSH2, TMEM185A, |
| | | | SIX5, CNPY3, RAXE, GNB2, RPL14, |
| | | | ATXN8, ISR, TTR, EP400, GIGYF2, |
| | | | OGG1, STC1, CNDP1, C10ORF2, |
| | | | MAML3, DKC1, PAXIP1, CASK, |
| | | | MAPT, SP1, POLG, AFF2, THBS1, |
| | | | TP53, ESR1, CGGBP1, ABT1, KLK3, |
| | | | PRNP, JUN, KCNN3, BAX, FRAXA, |
| | | | KBTBD10, MBNL1, RAD51, |
| | | | NCOA3, ERDA1, TSC1, COMP, |
| | | | GGLC, RRAD, MSH3, DRD2, CD44, |
| | | | CTCF, CCND1, CLSPN, MEF2A, |
| | | | PTPRU, GAPDH, TRIM22, WT1, |
| | | | AHR, GPX1, TPMT, NDP, ARX, |
| | | | TYR, EGRI, UNG, NUMBL, FABP2, |
| | | | EN2, CRYGC, SRP14, CRYGB, |
| | | | PDCD1,HOXA1, ATXN2L, PMS2, |
| | | | GLA, CBL, FTH1, IL12RB2, OTX2, |
| | | | HOXA5, POLG2, DLX2, AHRR, |
| | | | MANF, RMEM158, see also |
| | | | 20110016540 |
| Turner's Syndrome (XO) | Various - | | Monosomy X |
| | reproductive | | |
| | organs, and sex | | |
| | characteristics, | | |
| | vasculature | | |
| Tuberous Sclerosis | CNS, heart, | | TSC1, TSC2 |
| | kidneys | | |
| Usher syndrome (Types I, II, and | Ears, eyes | | ABHD12, CDH23, CIB2, CLRN1, |
| III) | | | DFNB31, GPR98, HARS, MYO7A, |
| | | | PCDH15, USH1C, USH1G, USH2A, |
| | | | USH11A, those described in |
| | | | WO2015134812A1 |
| Velocardiofacial syndrome (aka | Various - | | Many genes are deleted, COM, TBX1, |
| 22q11.2 deletion syndrome, | skeletal, heart, | | and other are associated with |
| DiGeorge syndrome, conotruncal | kidney, immune | | symptoms |
| anomaly face syndrome (CTAF), | system, brain | | |
| autosomal dominant Opitz G/BB | | | |
| syndrome or Cayler cardiofacial | | | |
| syndrome) | | | |
| Von Gierke's Disease (Glycogen | Glycogen | Various - liver, | G6PC and SLC37A4 |
| Storage Disease type I) | Storage disease | kidney | |
| Von Hippel-Lindau Syndrome | Various - cell | CNS, Kidney, | VHL |
| | growth | Eye, visceral | |
| | regulation | organs | |
| | disorder | | |
| Von Willebrand Disease (Types | blood | | VWF |
| I, II and III) | | | |
| Wilson Disease | Various - | Liver, brains, | ATP7B |
| | Copper Storage | eyes, other | |
| | Disease | tissues where | |
| | | copper builds up | |
| Wiskott-Aldrich Syndrome | Immune System | | WAS |
| Xeroderma Pigmentosum | Skin | Nervous system | POLH |
| XXX Syndrome | Endocrine, brain | | X chromosome trisomy |

In one embodiment, the compositions, systems, or components thereof can be used treat or prevent a disease in a subject by modifying one or more genes associated with one or more cellular functions, such as any one or more of those in Table 4B. In one embodiment, the disease is a genetic disease or disorder. In some of embodiments, the composition, system, or component thereof can modify one or more genes or polynucleotides associated with one or more genetic diseases such as any set forth in Table 4B.

TABLE 4B
Exemplary Genes controlling Cellular Functions

| CELLULAR FUNCTION | GENES |
|---|---|
| PI3K/AKT Signaling | PRKCE; ITGAM; ITGA5; IRAK1; PRKAA2; EIF2AK2; PTEN; EIF4E; |
| | PRKCZ; GRK6; MAPK1; TSC1; PLK1; AKT2; IKBKB; PIK3CA; CDK8; |
| | CDKN1B; NFKB2; BCL2; PIK3CB; PPP2R1A; MAPK8; BCL2L1; MAPK3; |
| | TSC2; |
| | ITGA1; KRAS; EIF4EBP1; RELA; PRKCD; NOS3; |
| | PRKAA1; MAPK9; CDK2; PPP2CA; PIM1; ITGB7; |
| | YWHAZ; ILK; TP53; RAF1; IKBKG; RELB; DYRK1A; |
| | CDKN1A; ITGB1; MAP2K2; JAK1; AKT1; JAK2; PIK3R1; |
| | CHUK; PDPK1; PPP2R5C; CTNNB1; MAP2K1; NFKB1; |
| | PAK3; ITGB3; CCND1; GSK3A; FRAP1; SFN; ITGA2; |
| | TTK; CSNK1A1; BRAF; GSK3B; AKT3; FOXO1; SGK; |
| | HSP90AA1; RPS6KB1 |
| ERK/MAPK Signaling | PRKCE; ITGAM; ITGA5; HSPB1; IRAK1; PRKAA2; |
| | EIF2AK2; RAC1; RAP1A; TLN1; EIF4E; ELK1; GRK6; |
| | MAPK1; RAC2; PLK1; AKT2; PIK3CA; CDK8; CREB1; |
| | PRKCI; PTK2; FOS; RPS6KA4; PIK3CB; PPP2R1A; |
| | PIK3C3; MAPK8; MAPK3; ITGA1; ETS1; KRAS; MYCN; |
| | EIF4EBP1; PPARG; PRKCD; PRKAA1; MAPK9; SRC; |
| | CDK2; PPP2CA; PIM1; PIK3C2A; ITGB7; YWHAZ; |
| | PPP1CC; KSR1; PXN; RAF1; FYN; DYRK1A; ITGB1; |
| | MAP2K2; PAK4; PIK3R1; STAT3; PPP2R5C; MAP2K1; |
| | PAK3; ITGB3; ESR1; ITGA2; MYC; TTK; CSNK1A1; |
| | CRKL; BRAF; ATF4; PRKCA; SRF; STAT1; SGK |
| Glucocorticoid Receptor | RAC1; TAF4B; EP300; SMAD2; TRAF6; PCAF; ELK1; |
| Signaling | MAPK1; SMAD3; AKT2; IKBKB; NCOR2; UBE2I; |
| | PIK3CA; CREB1; FOS; HSPA5; NFKB2; BCL2; |
| | MAP3K14; STAT5B; PIK3CB; PIK3C3; MAPK8; BCL2L1; |
| | MAPK3; TSC22D3; MAPK10; NRIP1; KRAS; MAPK13; |
| | RELA; STAT5A; MAPK9; NOS2A; PBX1; NR3C1; |
| | PIK3C2A; CDKN1C; TRAF2; SERPINE1; NCOA3; |
| | MAPK14; TNF; RAF1; IKBKG; MAP3K7; CREBBP; |
| | CDKN1A; MAP2K2; JAK1; IL8; NCOA2; AKT1; JAK2; |
| | PIK3R1; CHUK; STAT3; MAP2K1; NFKB1; TGFBR1; |
| | ESR1; SMAD4; CEBPB; JUN; AR; AKT3; CCL2; MMP1; |
| | STAT1; IL6; HSP90AA1 |
| Axonal Guidance Signaling | PRKCE; ITGAM; ROCK1; ITGA5; CXCR4; ADAM12; |
| | IGF1; RAC1; RAP1A; EIF4E; PRKCZ; NRP1; NTRK2; |
| | ARHGEF7; SMO; ROCK2; MAPK1; PGF; RAC2; |
| | PTPN11; GNAS; AKT2; PIK3CA; ERBB2; PRKCI; PTK2; |
| | CFL1; GNAQ; PIK3CB; CXCL12; PIK3C3; WNT11; |
| | PRKD1; GNB2L1; ABL1; MAPK3; ITGA1; KRAS; RHOA; |
| | PRKCD; PIK3C2A; ITGB7; GLI2; PXN; VASP; RAF1; |
| | FYN; ITGB1; MAP2K2; PAK4; ADAM17; AKT1; PIK3R1; |
| | GLI1; WNT5A; ADAM10; MAP2K1; PAK3; ITGB3; |
| | CDC42; VEGFA; ITGA2; EPHA8; CRKL; RND1; GSK3B; |
| | AKT3; PRKCA |
| Ephrin Receptor Signaling | PRKCE; ITGAM; ROCK1; ITGA5; CXCR4; IRAK1; |
| Actin Cytoskeleton | PRKAA2; EIF2AK2; RAC1; RAP1A; GRK6; ROCK2; |
| Signaling | MAPK1; PGF; RAC2; PTPN11; GNAS; PLK1; AKT2; |
| | DOK1; CDK8; CREB1; PTK2; CFL1; GNAQ; MAP3K14; |
| | CXCL12; MAPK8; GNB2L1; ABL1; MAPK3; ITGA1; |
| | KRAS; RHOA; PRKCD; PRKAA1; MAPK9; SRC; CDK2; |
| | PIM1; ITGB7; PXN; RAF1; FYN; DYRK1A; ITGB1; |
| | MAP2K2; PAK4; AKT1; JAK2; STAT3; ADAM10; |
| | MAP2K1; PAK3; ITGB3; CDC42; VEGFA; ITGA2; |
| | EPHA8; TTK; CSNK1A1; CRKL; BRAF; PTPN13; ATF4; |
| | AKT3; SGK |
| | ACTN4; PRKCE; ITGAM; ROCK1; ITGA5; IRAK1; |
| | PRKAA2; EIF2AK2; RAC1; INS; ARHGEF7; GRK6; |
| | ROCK2; MAPK1; RAC2; PLK1; AKT2; PIK3CA; CDK8; |
| | PTK2; CFL1; PIK3CB; MYH9; DIAPH1; PIK3C3; MAPK8; |
| | F2R; MAPK3; SLC9A1; ITGA1; KRAS; RHOA; PRKCD; |
| | PRKAA1; MAPK9; CDK2; PIM1; PIK3C2A; ITGB7; |
| | PPP1CC; PXN; VIL2; RAF1; GSN; DYRK1A; ITGB1; |
| | MAP2K2; PAK4; PIP5K1A; PIK3R1; MAP2K1; PAK3; |
| | ITGB3; CDC42; APC; ITGA2; TTK; CSNK1A1; CRKL; |
| | BRAF; VAV3; SGK |
| Huntington's Disease | PRKCE; IGF1; EP300; RCOR1; PRKCZ; HDAC4; TGM2; |
| Signaling | MAPK1; CAPNS1; AKT2; EGFR; NCOR2; SP1; CAPN2; |
| | PIK3CA; HDAC5; CREB1; PRKC1; HSPA5; REST; |
| | GNAQ; PIK3CB; PIK3C3; MAPK8; IGF1R; PRKD1; |
| | GNB2L1; BCL2L1; CAPN1; MAPK3; CASP8; HDAC2; |
| | HDAC7A; PRKCD; HDAC11; MAPK9; HDAC9; PIK3C2A; |
| | HDAC3; TP53; CASP9; CREBBP; AKT1; PIK3R1; |
| | PDPK1; CASP1; APAF1; FRAP1; CASP2; JUN; BAX; |
| | ATF4; AKT3; PRKCA; CLTC; SGK; HDAC6; CASP3 |
| Apoptosis Signaling | PRKCE; ROCK1; BID; IRAK1; PRKAA2; EIF2AK2; BAK1; |
| | BIRC4; GRK6; MAPK1; CAPNS1; PLK1; AKT2; IKBKB; |
| | CAPN2; CDK8; FAS; NFKB2; BCL2; MAP3K14; MAPK8; |
| | BCL2L1; CAPN1; MAPK3; CASP8; KRAS; RELA; |
| | PRKCD; PRKAA1; MAPK9; CDK2; PIM1; TP53; TNF; |
| | RAF1; IKBKG; RELB; CASP9; DYRK1A; MAP2K2; |
| | CHUK; APAF1; MAP2K1; NFKB1; PAK3; LMNA; CASP2; |
| | BIRC2; TTK; CSNK1A1; BRAF; BAX; PRKCA; SGK; |
| | CASP3; BIRC3; PARP1 |
| B Cell Receptor Signaling | RAC1; PTEN; LYN; ELK1; MAPK1; RAC2; PTPN11; |
| | AKT2; IKBKB; PIK3CA; CREB1; SYK; NFKB2; CAMK2A; |
| | MAP3K14; PIK3CB; PIK3C3; MAPK8; BCL2L1; ABL1; |
| | MAPK3; ETS1; KRAS; MAPK13; RELA; PTPN6; MAPK9; |
| | EGR1; PIK3C2A; BTK; MAPK14; RAF1; IKBKG; RELB; |
| | MAP3K7; MAP2K2; AKT1; PIK3R1; CHUK; MAP2K1; |
| | NFKB1; CDC42; GSK3A; FRAP1; BCL6; BCL10; JUN; |
| | GSK3B; ATF4; AKT3; VAV3; RPS6KB1 |
| Leukocyte Extravasation | ACTN4; CD44; PRKCE; ITGAM; ROCK1; CXCR4; CYBA; |
| Signaling | RAC1; RAP1A; PRKCZ; ROCK2; RAC2; PTPN11; |
| | MMP14; PIK3CA; PRKCI; PTK2; PIK3CB; CXCL12; |
| | PIK3C3; MAPK8; PRKD1; ABL1; MAPK10; CYBB; |
| | MAPK13; RHOA; PRKCD; MAPK9; SRC; PIK3C2A; BTK; |
| | MAPK14; NOX1; PXN; VIL2; VASP; ITGB1; MAP2K2; |
| | CTNND1; PIK3R1; CTNNB1; CLDN1; CDC42; F11R; ITK; |
| | CRKL; VAV3; CTTN; PRKCA; MMP1; MMP9 |
| Integrin Signaling | ACTN4; ITGAM; ROCK1; ITGA5; RAC1; PTEN; RAP1A; |
| | TLN1; ARHGEF7; MAPK1; RAC2; CAPNS1; AKT2; |
| | CAPN2; PIK3CA; PTK2; PIK3CB; PIK3C3; MAPK8; |
| | CAV1; CAPN1; ABL1; MAPK3; ITGA1; KRAS; RHOA; |
| | SRC; PIK3C2A; ITGB7; PPP1CC; ILK; PXN; VASP; |
| | RAF1; FYN; ITGB1; MAP2K2; PAK4; AKT1; PIK3R1; |
| | TNK2; MAP2K1; PAK3; ITGB3; CDC42; RND3; ITGA2; |
| | CRKL; BRAF; GSK3B; AKT3 |
| Acute Phase Response | IRAK1; SOD2; MYD88; TRAF6; ELK1; MAPK1; PTPN11; |
| Signaling | AKT2; IKBKB; PIK3CA; FOS; NFKB2; MAP3K14; |
| | PIK3CB; MAPK8; RIPK1; MAPK3; IL6ST; KRAS; |
| | MAPK13; IL6R; RELA; SOCS1; MAPK9; FTL; NR3C1; |
| | TRAF2; SERPINE1; MAPK14; TNF; RAF1; PDK1; |
| | IKBKG; RELB; MAP3K7; MAP2K2; AKT1; JAK2; PIK3R1; |
| | CHUK; STAT3; MAP2K1; NFKB1; FRAP1; CEBPB; JUN; |
| | AKT3; IL1R1; IL6 |
| PTEN Signaling | ITGAM; ITGA5; RAC1; PTEN; PRKCZ; BCL2L11; |
| | MAPK1; RAC2; AKT2; EGFR; IKBKB; CBL; PIK3CA; |
| | CDKN1B; PTK2; NFKB2; BCL2; PIK3CB; BCL2L1; |
| | MAPK3; ITGA1; KRAS; ITGB7; ILK; PDGFRB; INSR; |
| | RAF1; IKBKG; CASP9; CDKN1A; ITGB1; MAP2K2; |
| | AKT1; PIK3R1; CHUK; PDGFRA; PDPK1; MAP2K1; |
| | NFKB1; ITGB3; CDC42; CCND1; GSK3A; ITGA2; |
| | GSK3B; AKT3; FOXO1; CASP3; RPS6KB1 |
| p53 Signaling | PTEN; EP300; BBC3; PCAF; FASN; BRCA1; GADD45A; |
| Aryl Hydrocarbon Receptor | BIRC5; AKT2; PIK3CA; CHEK1; TP53INP1; BCL2; |
| Signaling | PIK3CB; PIK3C3; MAPK8; THBS1; ATR; BCL2L1; E2F1; |
| | PMAIP1; CHEK2; TNFRSF10B; TP73; RB1; HDAC9; |
| | CDK2; PIK3C2A; MAPK14; TP53; LRDD; CDKN1A; |
| | HIPK2; AKT1; PIK3R1; RRM2B; APAF1; CTNNB1; |
| | SIRT1; CCND1; PRKDC; ATM; SFN; CDKN2A; JUN; |
| | SNAI2; GSK3B; BAX; AKT3 |
| | HSPB1; EP300; FASN; TGM2; RXRA; MAPK1; NQO1; |
| | NCOR2; SP1; ARNT; CDKN1B; FOS; CHEK1; |
| | SMARCA4; NFKB2; MAPK8; ALDH1A1; ATR; E2F1; |
| | MAPK3; NRIP1; CHEK2; RELA; TP73; GSTP1; RB1; |
| | SRC; CDK2; AHR; NFE2L2; NCOA3; TP53; TNF; |
| | CDKN1A; NCOA2; APAF1; NFKB1; CCND1; ATM; ESR1; |
| | CDKN2A; MYC; JUN; ESR2; BAX; IL6; CYP1B1; |
| | HSP90AA1 |
| Xenobiotic Metabolism | PRKCE; EP300; PRKCZ; RXRA; MAPK1; NQO1; |
| Signaling | NCOR2; PIK3CA; ARNT; PRKCI; NFKB2; CAMK2A; |
| | PIK3CB; PPP2R1A; PIK3C3; MAPK8; PRKD1; |
| | ALDH1A1; MAPK3; NRIP1; KRAS; MAPK13; PRKCD; |
| | GSTP1; MAPK9; NOS2A; ABCB1; AHR; PPP2CA; FTL; |
| | NFE2L2; PIK3C2A; PPARGC1A; MAPK14; TNF; RAF1; |
| | CREBBP; MAP2K2; PIK3R1; PPP2R5C; MAP2K1; |
| | NFKB1; KEAP1; PRKCA; EIF2AK3; IL6; CYP1B1; |
| | HSP90AA1 |
| SAPK/JNK Signaling | PRKCE; IRAK1; PRKAA2; EIF2AK2; RAC1; ELK1; |
| | GRK6; MAPK1; GADD45A; RAC2; PLK1; AKT2; PIK3CA; |
| | FADD; CDK8; PIK3CB; PIK3C3; MAPK8; RIPK1; |
| | GNB2L1; IRS1; MAPK3; MAPK10; DAXX; KRAS; |
| | PRKCD; PRKAA1; MAPK9; CDK2; PIM1; PIK3C2A; |
| | TRAF2; TP53; LCK; MAP3K7; DYRK1A; MAP2K2; |
| | PIK3R1; MAP2K1; PAK3; CDC42; JUN; TTK; CSNK1A1; |
| | CRKL; BRAF; SGK |
| PPAr/RXR Signaling | PRKAA2; EP300; INS; SMAD2; TRAF6; PPARA; FASN; |
| | RXRA; MAPK1; SMAD3; GNAS; IKBKB; NCOR2; |
| | ABCA1; GNAQ; NFKB2; MAP3K14; STAT5B; MAPK8; |
| | IRS1; MAPK3; KRAS; RELA; PRKAA1; PPARGC1A; |
| | NCOA3; MAPK14; INSR; RAF1; IKBKG; RELB; MAP3K7; |
| | CREBBP; MAP2K2; JAK2; CHUK; MAP2K1; NFKB1; |
| | TGFBR1; SMAD4; JUN; IL1R1; PRKCA; IL6; HSP90AA1; |
| | ADIPOQ |
| NF-KB Signaling | IRAK1; EIF2AK2; EP300; INS; MYD88; PRKCZ; TRAF6; |
| | TBK1; AKT2; EGFR; IKBKB; PIK3CA; BTRC; NFKB2; |
| | MAP3K14; PIK3CB; PIK3C3; MAPK8; RIPK1; HDAC2; |
| | KRAS; RELA; PIK3C2A; TRAF2; TLR4; PDGFRB; TNF; |
| | INSR; LCK; IKBKG; RELB; MAP3K7; CREBBP; AKT1; |
| | PIK3R1; CHUK; PDGFRA; NFKB1; TLR2; BCL10; |
| | GSK3B; AKT3; TNFAIP3; IL1R1 |
| Neuregulin Signaling | ERBB4; PRKCE; ITGAM; ITGA5; PTEN; PRKCZ; ELK1; |
| Wnt & Beta catenin | MAPK1; PTPN11; AKT2; EGFR; ERBB2; PRKCI; |
| Signaling | CDKN1B; STAT5B; PRKD1; MAPK3; ITGA1; KRAS; |
| | PRKCD; STAT5A; SRC; ITGB7; RAF1; ITGB1; MAP2K2; |
| | ADAM17; AKT1; PIK3R1; PDPK1; MAP2K1; ITGB3; |
| | EREG; FRAP1; PSEN1; ITGA2; MYC; NRG1; CRKL; |
| | AKT3; PRKCA; HSP90AA1; RPS6KB1 |
| | CD44; EP300; LRP6; DVL3; CSNK1E; GJA1; SMO; |
| | AKT2; PIN1; CDH1; BTRC; GNAQ; MARK2; PPP2R1A; |
| | WNT11; SRC; DKK1; PPP2CA; SOX6; SFRP2; ILK; |
| | LEF1; SOX9; TP53; MAP3K7; CREBBP; TCF7L2; AKT1; |
| | PPP2R5C; WNT5A; LRP5; CTNNB1; TGFBR1; CCND1; |
| | GSK3A; DVL1; APC; CDKN2A; MYC; CSNK1A1; GSK3B; |
| | AKT3; SOX2 |
| Insulin Receptor Signaling | PTEN; INS; EIF4E; PTPN1; PRKCZ; MAPK1; TSC1; |
| | PTPN11; AKT2; CBL; PIK3CA; PRKCI; PIK3CB; PIK3C3; |
| | MAPK8; IRS1; MAPK3; TSC2; KRAS; EIF4EBP1; |
| | SLC2A4; PIK3C2A; PPP1CC; INSR; RAF1; FYN; |
| | MAP2K2; JAK1; AKT1; JAK2; PIK3R1; PDPK1; MAP2K1; |
| | GSK3A; FRAP1; CRKL; GSK3B; AKT3; FOXO1; SGK; |
| | RPS6KB1 |
| IL-6 Signaling | HSPB1; TRAF6; MAPKAPK2; ELK1; MAPK1; PTPN11; |
| | IKBKB; FOS; NFKB2; MAP3K14; MAPK8; MAPK3; |
| | MAPK10; IL6ST; KRAS; MAPK13; IL6R; RELA; SOCS1; |
| | MAPK9; ABCB1; TRAF2; MAPK14; TNF; RAF1; IKBKG; |
| | RELB; MAP3K7; MAP2K2; IL8; JAK2; CHUK; STAT3; |
| | MAP2K1; NFKB1; CEBPB; JUN; IL1R1; SRF; IL6 |
| Hepatic Cholestasis | PRKCE; IRAK1; INS; MYD88; PRKCZ; TRAF6; PPARA; |
| | RXRA; IKBKB; PRKCI; NFKB2; MAP3K14; MAPK8; |
| | PRKD1; MAPK10; RELA; PRKCD; MAPK9; ABCB1; |
| | TRAF2; TLR4; TNF; INSR; IKBKG; RELB; MAP3K7; IL8; |
| | CHUK; NR1H2; TJP2; NFKB1; ESR1; SREBF1; FGFR4; |
| | JUN; IL1R1; PRKCA; IL6 |
| IGF-1 Signaling | IGF1; PRKCZ; ELK1; MAPK1; PTPN11; NEDD4; AKT2; |
| | PIK3CA; PRKCI; PTK2; FOS; PIK3CB; PIK3C3; MAPK8; |
| | IGF1R; IRS1; MAPK3; IGFBP7; KRAS; PIK3C2A; |
| | YWHAZ; PXN; RAF1; CASP9; MAP2K2; AKT1; PIK3R1; |
| | PDPK1; MAP2K1; IGFBP2; SFN; JUN; CYR61; AKT3; |
| | FOXO1; SRF; CTGF; RPS6KB1 |
| NRF2-mediated Oxidative | PRKCE; EP300; SOD2; PRKCZ; MAPK1; SQSTM1; |
| Stress Response | NQO1; PIK3CA; PRKCI; FOS; PIK3CB; PIK3C3; MAPK8; |
| | PRKD1; MAPK3; KRAS; PRKCD; GSTP1; MAPK9; FTL; |
| | NFE2L2; PIK3C2A; MAPK14; RAF1; MAP3K7; CREBBP; |
| | MAP2K2; AKT1; PIK3R1; MAP2K1; PPIB; JUN; KEAP1; |
| | GSK3B; ATF4; PRKCA; EIF2AK3; HSP90AA1 |
| Hepatic Fibrosis/Hepatic | EDN1; IGF1; KDR; FLT1; SMAD2; FGFR1; MET; PGF; |
| Stellate Cell Activation | SMAD3; EGFR; FAS; CSF1; NFKB2; BCL2; MYH9; |
| | IGF1R; IL6R; RELA; TLR4; PDGFRB; TNF; RELB; IL8; |
| | PDGFRA; NFKB1; TGFBR1; SMAD4; VEGFA; BAX; |
| | ILIR1; CCL2; HGF; MMP1; STAT1; IL6; CTGF; MMP9 |
| PPAR Signaling | EP300; INS; TRAF6; PPARA; RXRA; MAPK1; IKBKB; |
| | NCOR2; FOS; NFKB2; MAP3K14; STAT5B; MAPK3; |
| | NRIP1; KRAS; PPARG; RELA; STAT5A; TRAF2; |
| | PPARGC1A; PDGFRB; TNF; INSR; RAF1; IKBKG; |
| | RELB; MAP3K7; CREBBP; MAP2K2; CHUK; PDGFRA; |
| | MAP2K1; NFKB1; JUN; IL1R1; HSP90AA1 |
| Fc Epsilon RI Signaling | PRKCE; RAC1; PRKCZ; LYN; MAPK1; RAC2; PTPN11; |
| | AKT2; PIK3CA; SYK; PRKCI; PIK3CB; PIK3C3; MAPK8; |
| | PRKD1; MAPK3; MAPK10; KRAS; MAPK13; PRKCD; |
| | MAPK9; PIK3C2A; BTK; MAPK14; TNF; RAF1; FYN; |
| | MAP2K2; AKT1; PIK3R1; PDPK1; MAP2K1; AKT3; |
| | VAV3; PRKCA |
| G-Protein Coupled | PRKCE; RAP1A; RGS16; MAPK1; GNAS; AKT2; IKBKB; |
| Receptor Signaling | PIK3CA; CREB1; GNAQ; NFKB2; CAMK2A; PIK3CB; |
| | PIK3C3; MAPK3; KRAS; RELA; SRC; PIK3C2A; RAF1; |
| | IKBKG; RELB; FYN; MAP2K2; AKT1; PIK3R1; CHUK; |
| | PDPK1; STAT3; MAP2K1; NFKB1; BRAF; ATF4; AKT3; |
| | PRKCA |
| Inositol Phosphate | PRKCE; IRAK1; PRKAA2; EIF2AK2; PTEN; GRK6; |
| Metabolism | MAPK1; PLK1; AKT2; PIK3CA; CDK8; PIK3CB; PIK3C3; |
| | MAPK8; MAPK3; PRKCD; PRKAA1; MAPK9; CDK2; |
| | PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; |
| | PIK3C3; MAPK8; CAV1; ABL1; MAPK3; KRAS; SRC; |
| | PIK3C2A; PDGFRB; RAF1; MAP2K2; JAK1; JAK2; |
| | PIK3R1; PDGFRA; STAT3; SPHK1; MAP2K1; MYC; |
| | JUN; CRKL; PRKCA; SRF; STAT1; SPHK2 |
| VEGF Signaling | ACTN4; ROCK1; KDR; FLT1; ROCK2; MAPK1; PGF; |
| | AKT2; PIK3CA; ARNT; PTK2; BCL2; PIK3CB; PIK3C3; |
| | BCL2L1; MAPK3; KRAS; HIF1A; NOS3; PIK3C2A; PXN; |
| | RAF1; MAP2K2; ELAVL1; AKT1; PIK3R1; MAP2K1; SFN; |
| | VEGFA; AKT3; FOXO1; PRKCA |
| Natural Killer Cell | PRKCE; RAC1; PRKCZ; MAPK1; RAC2; PTPN11; |
| Signaling | KIR2DL3; AKT2; PIK3CA; SYK; PRKCI; PIK3CB; |
| | PIK3C3; PRKD1; MAPK3; KRAS; PRKCD; PTPN6; |
| | PIK3C2A; LCK; RAF1; FYN; MAP2K2; PAK4; AKT1; |
| | PIK3R1; MAP2K1; PAK3; AKT3; VAV3; PRKCA |
| Cell Cycle: G1/S | HDAC4; SMAD3; SUV39H1; HDAC5; CDKN1B; BTRC; |
| Checkpoint Regulation | ATR; ABL1; E2F1; HDAC2; HDAC7A; RB1; HDAC11; |
| | HDAC9; CDK2; E2F2; HDAC3; TP53; CDKN1A; CCND1; |
| | E2F4; ATM; RBL2; SMAD4; CDKN2A; MYC; NRG1; |
| | GSK3B; RBL1; HDAC6 |
| T Cell Receptor Signaling | RAC1; ELK1; MAPK1; IKBKB; CBL; PIK3CA; FOS; |
| | NFKB2; PIK3CB; PIK3C3; MAPK8; MAPK3; KRAS; |
| | RELA; PIK3C2A; BTK; LCK; RAF1; IKBKG; RELB; FYN; |
| | MAP2K2; PIK3R1; CHUK; MAP2K1; NFKB1; ITK; BCL10; |
| | JUN; VAV3 |
| Death Receptor Signaling | CRADD; HSPB1; BID; BIRC4; TBK1; IKBKB; FADD; |
| | FAS; NFKB2; BCL2; MAP3K14; MAPK8; RIPK1; CASP8; |
| | DAXX; TNFRSF10B; RELA; TRAF2; TNF; IKBKG; RELB; |
| | CASP9; CHUK; APAF1; NFKB1; CASP2; BIRC2; CASP3; |
| | BIRC3 |
| FGF Signaling | RAC1; FGFR1; MET; MAPKAPK2; MAPK1; PTPN11; |
| | AKT2; PIK3CA; CREB1; PIK3CB; PIK3C3; MAPK8; |
| | MAPK3; MAPK13; PTPN6; PIK3C2A; MAPK14; RAF1; |
| | AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; |
| | STAT5B; PIK3CB; PIK3C3; GNB2L1; BCL2L1; MAPK3; |
| | ETS1; KRAS; RUNX1; PIM1; PIK3C2A; RAF1; MAP2K2; |
| | AKT1; JAK2; PIK3R1; STAT3; MAP2K1; CCND1; AKT3; |
| | STAT1 |
| Amyotrophic Lateral | BID; IGF1; RAC1; BIRC4; PGF; CAPNS1; CAPN2; |
| Sclerosis Signaling | PIK3CA; BCL2; PIK3CB; PIK3C3; BCL2L1; CAPN1; |
| | PIK3C2A; TP53; CASP9; PIK3R1; RAB5A; CASP1; |
| | APAF1; VEGFA; BIRC2; BAX; AKT3; CASP3; BIRC3 |
| JAK/Stat Signaling | PTPN1; MAPK1; PTPN11; AKT2; PIK3CA; STAT5B; |
| | PIK3CB; PIK3C3; MAPK3; KRAS; SOCS1; STAT5A; |
| | PTPN6; PIK3C2A; RAF1; CDKN1A; MAP2K2; JAK1; |
| | AKT1; JAK2; PIK3R1; STAT3; MAP2K1; FRAP1; AKT3; |
| | STAT1 |
| Nicotinate and Nicotinamide | PRKCE; IRAK1; PRKAA2; EIF2AK2; GRK6; MAPK1; |
| Metabolism | PLK1; AKT2; CDK8; MAPK8; MAPK3; PRKCD; PRKAA1; |
| | PBEF1; MAPK9; CDK2; PIM1; DYRK1A; MAP2K2; |
| | MAP2K1; PAK3; NT5E; TTK; CSNK1A1; BRAF; SGK |
| Chemokine Signaling | CXCR4; ROCK2; MAPK1; PTK2; FOS; CFL1; GNAQ; |
| | CAMK2A; CXCL12; MAPK8; MAPK3; KRAS; MAPK13; |
| | RHOA; CCR3; SRC; PPP1CC; MAPK14; NOX1; RAF1; |
| | MAP2K2; MAP2K1; JUN; CCL2; PRKCA |
| IL-2 Signaling | ELK1; MAPK1; PTPN11; AKT2; PIK3CA; SYK; FOS; |
| | STAT5B; PIK3CB; PIK3C3; MAPK8; MAPK3; KRAS; |
| | SOCS1; STAT5A; PIK3C2A; LCK; RAF1; MAP2K2; |
| | JAK1; AKT1; PIK3R1; MAP2K1; JUN; AKT3 |
| Synaptic Long Term | PRKCE; IGF1; PRKCZ; PRDX6; LYN; MAPK1; GNAS; |
| Depression | PRKCI; GNAQ; PPP2R1A; IGF1R; PRKD1; MAPK3; |
| | KRAS; GRN; PRKCD; NOS3; NOS2A; PPP2CA; |
| | YWHAZ; RAF1; MAP2K2; PPP2R5C; MAP2K1; PRKCA |
| Estrogen Receptor | TAF4B; EP300; CARM1; PCAF; MAPK1; NCOR2; |
| Signaling | SMARCA4; MAPK3; NRIP1; KRAS; SRC; NR3C1; |
| | HDAC3; PPARGC1A; RBM9; NCOA3; RAF1; CREBBP; |
| | MAP2K2; NCOA2; MAP2K1; PRKDC; ESR1; ESR2 |
| Protein Ubiquitination | TRAF6; SMURF1; BIRC4; BRCA1; UCHL1; NEDD4; |
| Pathway | CBL; UBE2I; BTRC; HSPA5; USP7; USP10; FBXW7; |
| | USP9X; STUB1; USP22; B2M; BIRC2; PARK2; USP8; |
| | USP1; VHL; HSP90AA1; BIRC3 |
| IL-10 Signaling | TRAF6; CCR1; ELK1; IKBKB; SP1; FOS; NFKB2; |
| | MAP3K14; MAPK8; MAPK13; RELA; MAPK14; TNF; |
| | IKBKG; RELB; MAP3K7; JAK1; CHUK; STAT3; NFKB1; |
| | JUN; IL1R1; IL6 |
| VDR/RXR Activation | PRKCE; EP300; PRKCZ; RXRA; GADD45A; HES1; |
| | NCOR2; SP1; PRKCI; CDKN1B; PRKD1; PRKCD; |
| | RUNX2; KLF4; YY1; NCOA3; CDKN1A; NCOA2; SPP1; |
| | LRP5; CEBPB; FOXO1; PRKCA |
| TGF-beta Signaling | EP300; SMAD2; SMURF1; MAPK1; SMAD3; SMAD1; |
| | FOS; MAPK8; MAPK3; KRAS; MAPK9; RUNX2; |
| | SERPINE1; RAF1; MAP3K7; CREBBP; MAP2K2; |
| | MAP2K1; TGFBR1; SMAD4; JUN; SMAD5 |
| Toll-like Receptor Signaling | IRAK1; EIF2AK2; MYD88; TRAF6; PPARA; ELK1; |
| | IKBKB; FOS; NFKB2; MAP3K14; MAPK8; MAPK13; |
| | RELA; TLR4; MAPK14; IKBKG; RELB; MAP3K7; CHUK; |
| | NFKB1; TLR2; JUN |
| p38 MAPK Signaling | HSPB1; IRAK1; TRAF6; MAPKAPK2; ELK1; FADD; FAS; |
| | CREB1; DDIT3; RPS6KA4; DAXX; MAPK13; TRAF2; |
| | MAPK14; TNF; MAP3K7; TGFBR1; MYC; ATF4; IL1R1; |
| | SRF; STAT1 |
| Neurotrophin/TRK Signaling | NTRK2; MAPK1; PTPN11; PIK3CA; CREB1; FOS; |
| | PIK3CB; PIK3C3; MAPK8; MAPK3; KRAS; PIK3C2A; |
| | RAF1; MAP2K2; AKT1; PIK3R1; PDPK1; MAP2K1; |
| | CDC42; JUN; ATF4 |
| FXR/RXR Activation | INS; PPARA; FASN; RXRA; AKT2; SDC1; MAPK8; |
| | APOB; MAPK10; PPARG; MTTP; MAPK9; PPARGC1A; |
| | TNF; CREBBP; AKT1; SREBF1; FGFR4; AKT3; FOXO1 |
| Synaptic Long Term | PRKCE; RAP1A; EP300; PRKCZ; MAPK1; CREB1; |
| Potentiation | PRKCI; GNAQ; CAMK2A; PRKD1; MAPK3; KRAS; |
| | PRKCD; PPP1CC; RAF1; CREBBP; MAP2K2; MAP2K1; |
| | ATF4; PRKCA |
| Calcium Signaling | RAP1A; EP300; HDAC4; MAPK1; HDAC5; CREB1; |
| | CAMK2A; MYH9; MAPK3; HDAC2; HDAC7A; HDAC11; |
| | HDAC9; HDAC3; CREBBP; CALR; CAMKK2; ATF4; |
| | HDAC6 |
| EGF Signaling | ELK1; MAPK1; EGFR; PIK3CA; FOS; PIK3CB; PIK3C3; |
| | MAPK8; MAPK3; PIK3C2A; RAF1; JAK1; PIK3R1; |
| | STAT3; MAP2K1; JUN; PRKCA; SRF; STAT1 |
| Hypoxia Signaling in the | EDN1; PTEN; EP300; NQO1; UBE2I; CREB1; ARNT; |
| Cardiovascular System | HIF1A; SLC2A4; NOS3; TP53; LDHA; AKT1; ATM; |
| | VEGFA; JUN; ATF4; VHL; HSP90AA1 |
| LPS/IL-1 Mediated Inhibition | IRAK1; MYD88; TRAF6; PPARA; RXRA; ABCA1; |
| of RXR Function | MAPK8; ALDH1A1; GSTP1; MAPK9; ABCB1; TRAF2; |
| | TLR4; TNF; MAP3K7; NR1H2; SREBF1; JUN; IL1R1 |
| LXR/RXR Activation | FASN; RXRA; NCOR2; ABCA1; NFKB2; IRF3; RELA; |
| | NOS2A; TLR4; TNF; RELB; LDLR; NR1H2; NFKB1; |
| | SREBF1; IL1R1; CCL2; IL6; MMP9 |
| Amyloid Processing | PRKCE; CSNK1E; MAPK1; CAPNS1; AKT2; CAPN2; |
| | CAPN1; MAPK3; MAPK13; MAPT; MAPK14; AKT1; |
| | PSEN1; CSNK1A1; GSK3B; AKT3; APP |
| IL-4 Signaling | AKT2; PIK3CA; PIK3CB; PIK3C3; IRS1; KRAS; SOCS1; |
| | PTPN6; NR3C1; PIK3C2A; JAK1; AKT1; JAK2; PIK3R1; |
| | FRAP1; AKT3; RPS6KB1 |
| Cell Cycle: G2/M DNA | EP300; PCAF; BRCA1; GADD45A; PLK1; BTRC; |
| Damage Checkpoint | CHEK1; ATR; CHEK2; YWHAZ; TP53; CDKN1A; |
| Regulation | PRKDC; ATM; SFN; CDKN2A |
| Nitric Oxide Signaling in the | KDR; FLT1; PGF; AKT2; PIK3CA; PIK3CB; PIK3C3; |
| Cardiovascular System | CAV1; PRKCD; NOS3; PIK3C2A; AKT1; PIK3R1; |
| | VEGFA; AKT3; HSP90AA1 |
| Purine Metabolism | NME2; SMARCA4; MYH9; RRM2; ADAR; EIF2AK4; |
| | PKM2; ENTPD1; RAD51; RRM2B; TJP2; RAD51C; |
| | NT5E; POLD1; NME1 |
| cAMP-mediated Signaling | RAP1A; MAPK1; GNAS; CREB1; CAMK2A; MAPK3; |
| | SRC; RAF1; MAP2K2; STAT3; MAP2K1; BRAF; ATF4 |
| Mitochondrial Dysfunction | SOD2; MAPK8; CASP8; MAPK10; MAPK9; CASP9; |
| Notch Signaling | PARK7; PSEN1; PARK2; APP; CASP3 |
| | HES1; JAG1; NUMB; NOTCH4; ADAM17; NOTCH2; |
| | PSEN1; NOTCH3; NOTCH1; DLL4 |
| Endoplasmic Reticulum | HSPA5; MAPK8; XBP1; TRAF2; ATF6; CASP9; ATF4; |
| Stress Pathway | EIF2AK3; CASP3 |
| Pyrimidine Metabolism | NME2; AICDA; RRM2; EIF2AK4; ENTPD1; RRM2B; |
| | NT5E; POLD1; NME1 |
| Parkinson's Signaling | UCHL1; MAPK8; MAPK13; MAPK14; CASP9; PARK7; |
| | PARK2; CASP3 |
| Cardiac & Beta Adrenergic | GNAS; GNAQ; PPP2R1A; GNB2L1; PPP2CA; PPP1CC; |
| Signaling | PPP2R5C |
| Glycolysis/Gluconeogenesis | HK2; GCK; GPI; ALDH1A1; PKM2; LDHA; HK1 |
| Interferon Signaling | IRF1; SOCS1; JAK1; JAK2; IFITM1; STAT1; IFIT3 |
| Sonic Hedgehog Signaling | ARRB2; SMO; GLI2; DYRK1A; GLI1; GSK3B; DYRK1B |
| Glycerophospholipid | PLD1; GRN; GPAM; YWHAZ; SPHK1; SPHK2 |
| Metabolism | |
| Phospholipid Degradation | PRDX6; PLD1; GRN; YWHAZ; SPHK1; SPHK2 |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision Repair | ERCC5; ERCC4; XPA; XPC; ERCC1 |
| Pathway | |
| Starch and Sucrose | UCHL1; HK2; GCK; GPI; HK1 |
| Metabolism | |
| Aminosugars Metabolism | NQO1; HK2; GCK; HK1 |
| Arachidonic Acid | PRDX6; GRN; YWHAZ; CYP1B1 |
| Metabolism | |
| Circadian Rhythm Signaling | CSNK1E; CREB1; ATF4; NR1D1 |
| Coagulation System | BDKRB1; F2R; SERPINE1; F3 |
| Dopamine Receptor | PPP2R1A; PPP2CA; PPP1CC; PPP2R5C |
| Signaling | |
| Glutathione Metabolism | IDH2; GSTP1; ANPEP; IDH1 |
| Glycerolipid Metabolism | ALDH1A1; GPAM; SPHK1; SPHK2 |
| Linoleic Acid Metabolism | PRDX6; GRN; YWHAZ; CYP1B1 |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDX1; TYR |
| Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of Xenobiotics | GSTP1; CYP1B1 |
| by Cytochrome p450 | |
| Methane Metabolism | PRDX6; PRDX1 |
| Phenylalanine Metabolism | PRDX6; PRDX1 |
| Propanoate Metabolism | ALDH1A1; LDHA |
| Selenoamino Acid | PRMT5; AHCY |
| Metabolism | |
| Sphingolipid Metabolism | SPHK1; SPHK2 |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated Oxidative | PRDX1 |
| Stress Response | |
| Pentose Phosphate | GPI |
| Pathway | |
| Pentose and Glucuronate | UCHL1 |
| Interconversions | |
| Retinol Metabolism | ALDH1A1 |
| Riboflavin Metabolism | TYR |
| Tyrosine Metabolism | PRMT5, TYR |
| Ubiquinone Biosynthesis | PRMT5 |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; Grk2; |
| | Trpa1; Pomc; Cgrp; Crf; Pka; Era; Nr2b; TRPM5; Prkaca; |
| | Prkacb; Prkar1a; Prkar2a |
| Mitochondrial Function | AIF; CytC; SMAC (Diablo); Aifm-1; Aifm-2 |
| Developmental Neurology | BMP-4; Chordin (Chrd); Noggin (Nog); WNT (Wnt2; |
| | Wnt2b; Wnt3a; Wnt4; Wnt5a; Wnt6; Wnt7b; Wnt8b; |
| | Wnt9a; Wnt9b; Wnt10a; Wnt10b; Wnt16); beta-catenin; |
| | Dkk-1; Frizzled related proteins; Otx-2; Gbx2; FGF-8; |
| | Reelin; Dab1; unc-86 (Pou4f1 or Brn3a); Numb; Reln |

In an aspect, the invention provides a method of individualized or personalized treatment of a genetic disease in a subject in need of such treatment comprising: (a) introducing one or more mutations ex vivo in a tissue, organ or a cell line, or in vivo in a transgenic non-human mammal, comprising delivering to cell(s) of the tissue, organ, cell or mammal a composition comprising the particle delivery system or the delivery system or the virus particle of any one of the above embodiment or the cell of any one of the above embodiment, wherein the specific mutations or precise sequence substitutions are or have been correlated to the genetic disease; (b) testing treatment(s) for the genetic disease on the cells to which the vector has been delivered that have the specific mutations or precise sequence substitutions correlated to the genetic disease; and (c) treating the subject based on results from the testing of treatment(s) of step (b).

### Infectious Diseases

In one embodiment, the composition, system(s) or component(s) thereof can be used to diagnose, prognose, treat, and/or prevent an infectious disease caused by a microorganism, such as bacteria, virus, fungi, parasites, or combinations thereof.

In one embodiment, the system(s) or component(s) thereof can be capable of targeting specific microorganism within a mixed population. Exemplary methods of such techniques are described in e.g., Gomaa A A, Klumpe H E, Luo M L, Selle K, Barrangou R, Beisel C L. 2014. Programmable removal of bacterial strains by use of genome-targeting composition, systems, mBio 5: e00928-13; Citorik R J, Mimee M, Lu T K. 2014. Sequence-specific antimicrobials using efficiently delivered RNA-guided nucleases. Nat Biotechnol 32:1141-1145, the teachings of which can be adapted for use with the compositions, systems, and components thereof described herein.

In one embodiment, the composition, system(s) and/or components thereof can be capable of targeting pathogenic and/or drug-resistant microorganisms, such as bacteria, virus, parasites, and fungi. In one embodiment, the composition, system(s) and/or components thereof can be capable of targeting and modifying one or more polynucleotides in a pathogenic microorganism such that the microorganism is less virulent, killed, inhibited, or is otherwise rendered incapable of causing disease and/or infecting and/or replicating in a host cell.

In one embodiment, the pathogenic bacteria that can be targeted and/or modified by the composition, system, (s) and/or component(s) thereof described herein include, but are not limited to, those of the genus Actinomyces (e.g., A. israelii), Bacillus (e.g., B. anthracis, B. cereus), Bactereoides (e.g., B. fragilis), Bartonella (B. henselae, B. quintana), Bordetella (B. pertussis), Borrelia (e.g., B. burgdorferi, B. garinii, B. afzelii, and B. recurreentis), Brucella (e.g., B. abortus, B. canis, B. melitensis, and B. suis), Campylobacter (e.g., C. jejuni), Chlamydia (e.g., C. pneumoniae and C. trachomatis), Chlamydophila (e.g., C. psittaci), Clostridium (e.g., C. botulinum, C. difficile, C. perfringens. C. tetani), Corynebacterium (e.g., C. diptheriae), Enterococcus (e.g., E. Faecalis, E. faecium), Ehrlichia (E. canis and E. chaffensis) Escherichia (e.g., E. coli), Francisella (e.g., F. tularensis), Haemophilus (e.g., H. influenzae), Helicobacter (H. pylori), Klebsiella (E.g., K. pneumoniae), Legionella (e.g., L. pneumophila), Leptospira (e.g., L. interrogans, L. santarosai, L. weilii, L. noguchii), Listereia (e.g., L. monocytogeenes), Mycobacterium (e.g., M. leprae, M. tuberculosis, M. ulcerans), Mycoplasma (M. pneumoniae), Neisseria (N. gonorrhoeae and N. menigitidis), Nocardia (e.g., N. asteeroides), Pseudomonas (P. aeruginosa), Rickettsia (R. rickettsia), Salmonella (S. typhi and S. typhimurium), Shigella (S. sonnei and S. dysenteriae), Staphylococcus (S. aureus, S. epidermidis, and S. saprophyticus), Streeptococcus (S. agalactiaee, S. pneumoniae, S. pyogenes), Treponema (T. pallidum), Ureeaplasma (e.g., U. urealyticum), Vibrio (e.g., V. cholerae), Yersinia (e.g., Y. pestis, Y. enteerocolitica, and Y. pseudotuberculosis).

In one embodiment, the pathogenic virus that can be targeted and/or modified by the composition, system, (s) and/or component(s) thereof described herein include, but are not limited to, a double-stranded DNA virus, a partly double-stranded DNA virus, a single-stranded DNA virus, a positive single-stranded RNA virus, a negative single-stranded RNA virus, or a double stranded RNA virus. In one embodiment, the pathogenic virus can be from the family Adenoviridae (e.g., Adenovirus), Herpeesviridae (e.g., Herpes simplex, type 1, Herpes simplex, type 2, Varicella-zoster virus, Epstein-Barr virus, Human cytomegalovirus, Human herpesvirus, type 8), Papillomaviridae (e.g., Human papillomavirus), Polyomaviridae (e.g., BK virus, JC virus), Poxviridae (e.g., smallpox), Hepadnaviridae (e.g., Hepatitis B), Parvoviridae (e.g., Parvovirus B19), Astroviridae (e.g., Human astrovirus), Caliciviridae (e.g., Norwalk virus), Picornaviridae (e.g., coxsackievirus, hepatitis A virus, poliovirus, rhinovirus), Coronaviridae (e.g., Severe acute respiratory syndrome-related coronavirus, strains: Severe acute respiratory syndrome virus, Severe acute respiratory syndrome coronavirus 2 (COVID-19)), Flaviviridae (e.g., Hepatitis C virus, yellow fever virus, dengue virus, West Nile virus, TBE virus), Togaviridae (e.g., Rubella virus), Hepeviridae (e.g., Hepatitis E virus), Retroviridae (Human immunodeficiency virus (HIV)), Orthomyxoviridae (e.g., Influenza virus), Arenaviridae (e.g., Lassa virus), Bunyaviridae (e.g., Crimean-Congo hemorrhagic fever virus, Hantaan virus), Filoviridae (e.g., Ebola virus and Marburg virus), Paramyxoviridae (e.g., Measles virus, Mumps virus, Parainfluenza virus, Respiratory syncytial virus), Rhabdoviridae (Rabies virus), Hepatits D virus, Reoviridae (e.g., Rotavirus, Orbivirus, Coltivirus, Banna virus).

In one embodiment, the pathogenic fungi that can be targeted and/or modified by the composition, system, (s) and/or component(s) thereof described herein include, but are not limited to, those of the genus Candida (e.g., C. albicans), Aspergillus (e.g., A. fumigatus, A. flavus, A. clavatus), Cryptococcus (e.g., C. neoformans, C. gattii), Histoplasma (H. capsulatum), Pneumocystis (e.g., P. jiroveecii), Stachybotrys (e.g., S. chartarum).

In one embodiment, the pathogenic parasites that can be targeted and/or modified by the composition, system(s) and/or component(s) thereof described herein include, but are not limited to, protozoa, helminths, and ectoparasites. In one embodiment, the pathogenic protozoa that can be targeted and/or modified by the composition, system(s) and/or component(s) thereof described herein include, but are not limited to, those from the groups Sarcodina (e.g., ameba such as Entamoeba), Mastigophora (e.g., flagellates such as Giardia and Leishmania), Cilophora (e.g., ciliates such as Balantidum), and sporozoa (e.g., plasmodium and cryptosporidium). In one embodiment, the pathogenic helminths that can be targeted and/or modified by the composition, system(s) and/or component(s) thereof described herein include, but are not limited to, flatworms (platyhelminths), thorny-headed worms (acanthoceephalins), and roundworms (nematodes). In one embodiment, the pathogenic ectoparasites that can be targeted and/or modified by the composition, system(s) and/or component(s) thereof described herein include, but are not limited to, ticks, fleas, lice, and mites.

In one embodiment, the pathogenic parasite that can be targeted and/or modified by the composition, system(s) and/or component(s) thereof described herein include, but are not limited to, Acanthamoeba spp., Balamuthia mandrillaris, Babesiosis spp. (e.g., Babesia B. divergens, B. bigemina, B. equi, B. microfti, B. duncani), Balantidiasis spp. (e.g., Balantidium coli), Blastocystis spp., Cryptosporidium spp., Cyclosporiasis spp. (e.g., Cyclospora cayetanensis), Dientamoebiasis spp. (e.g., Dientamoeba fragilis), Amoebiasis spp. (e.g., Entamoeba histolytica), Giardiasis spp. (e.g., Giardia lamblia), Isosporiasis spp. (e.g., Isospora belli), Leishmania spp., Naegleria spp. (e.g., Naegleria fowleri), Plasmodium spp. (e.g., Plasmodium falciparum, Plasmodium vivax, Plasmodium ovale curtisi, Plasmodium ovale wallikeri, Plasmodium malariae, Plasmodium knowlesi), Rhinosporidiosis spp. (e.g., Rhinosporidium seeberi), Sarcocystosis spp. (e.g., Sarcocystis bovihominis, Sarcocystis suihominis), Toxoplasma spp. (e.g., Toxoplasma gondii), Trichomonas spp. (e.g., Trichomonas vaginalis), Trypanosoma spp. (e.g., Trypanosoma brucei), Trypanosoma spp. (e.g., Trypanosoma cruzi), Tapeworm (e.g., Cestoda, Taenia multiceps, Taenia saginata, Taenia solium), Diphyllobothrium latum spp., Echinococcus spp. (e.g., Echinococcus granulosus, Echinococcus multilocularis, E. vogeli, E. oligarthrus), Hymenolepis spp. (e.g., Hymenolepis nana, Hymenolepis diminuta), Bertiella spp. (e.g., Bertiella mucronata, Bertiella studeri), Spirometra (e.g., Spirometra erinaceieuropaei), Clonorchis spp. (e.g., Clonorchis sinensis; Clonorchis viverrini), Dicrocoelium spp. (e.g., Dicrocoelium dendriticum), Fasciola spp. (e.g., Fasciola hepatica, Fasciola gigantica), Fasciolopsis spp. (e.g., Fasciolopsis buski), Metagonimus spp. (e.g., Metagonimus yokogawai), Metorchis spp. (e.g., Metorchis conjunctus), Opisthorchis spp. (e.g., Opisthorchis viverrini, Opisthorchis felineus), Clonorchis spp. (e.g., Clonorchis sinensis), Paragonimus spp. (e.g., Paragonimus westermani; Paragonimus africanus; Paragonimus caliensis; Paragonimus kellicotti; Paragonimus skrjabini; Paragonimus uterobilateralis), Schistosoma sp., Schistosoma spp. (e.g., Schistosoma mansoni, Schistosoma haematobium, Schistosoma japonicum, Schistosoma mekongi, and Schistosoma intercalatum), Echinostoma spp. (e.g., E. echinatum), Trichobilharzia spp. (e.g., Trichobilharzia regent), Ancylostoma spp. (e.g., Ancylostoma duodenale), Necator spp. (e.g., Necator americanus), Angiostrongylus spp., Anisakis spp., Ascaris spp. (e.g., Ascaris lumbricoides), Baylisascaris spp. (e.g., Baylisascaris procyonis), Brugia spp. (e.g., Brugia malayi, Brugia timori), Dioctophyme spp. (e.g., Dioctophyme renale), Dracunculus spp. (e.g., Dracunculus medinensis), Enterobius spp. (e.g., Enterobius vermicularis, Enterobius gregorii), Gnathostoma spp. (e.g., Gnathostoma spinigerum, Gnathostoma hispidum), Halicephalobus spp. (e.g., Halicephalobus gingivalis), Loa loa spp. (e.g., Loa loa filaria), Mansonella spp. (e.g., Mansonella streptocerca), Onchocerca spp. (e.g., Onchocerca volvulus), Strongyloides spp. (e.g., Strongyloides stercoralis), Thelazia spp. (e.g., Thelazia californiensis, Thelazia callipaeda), Toxocara spp. (e.g., Toxocara canis, Toxocara cati, Toxascaris leonine), Trichinella spp. (e.g., Trichinella spiralis, Trichinella britovi, Trichinella nelsoni, Trichinella nativa), Trichuris spp. (e.g., Trichuris trichiura, Trichuris vulpis), Wuchereria spp. (e.g., Wuchereria bancrofti), Dermatobia spp. (e.g., Dermatobia hominis), Tunga spp. (e.g., Tunga penetrans), Cochliomyia spp. (e.g., Cochliomyia hominivorax), Linguatula spp. (e.g., Linguatula serrata), Archiacanthocephala sp., Moniliformis sp. (e.g., Moniliformis moniliformis), Pediculus spp. (e.g., Pediculus humanus capitis, Pediculus humanus humanus), Pthirus spp. (e.g., Pthirus pubis), Arachnida spp. (e.g., Trombiculidae, Ixodidae, Argaside), Siphonaptera spp (e.g., Siphonaptera: Pulicinae), Cimicidae spp. (e.g., Cimex lectularius and Cimex hemipterus), Diptera spp., Demodex spp. (e.g., Demodex folliculorum/brevis/canis), Sarcoptes spp. (e.g., Sarcoptes scabiei), Dermanyssus spp. (e.g., Dermanyssus gallinae), Ornithonyssus spp. (e.g., Ornithonyssus sylviarum, Ornithonyssus bursa, Ornithonyssus bacoti), Laelaps spp. (e.g., Laelaps echidnina), Liponyssoides spp. (e.g., Liponyssoides sanguineus).

In one embodiment the gene targets can be any of those as set forth in Table 1 of Strich and Chertow, 2019. J. Clin. Microbio. 57:4 e01307-18, which is incorporated herein as if expressed in its entirety herein.

In one embodiment, the method can include delivering a composition, system, and/or component thereof to a pathogenic organism described herein, allowing the composition, system, and/or component thereof to specifically bind and modify one or more targets in the pathogenic organism, whereby the modification kills, inhibits, reduces the pathogenicity of the pathogenic organism, or otherwise renders the pathogenic organism non-pathogenic. In one embodiment, delivery of the composition, system, occurs in vivo (i.e., in the subject being treated). In one embodiment occurs by an intermediary, such as microorganism or phage that is non-pathogenic to the subject but is capable of transferring polynucleotides and/or infecting the pathogenic microorganism. In one embodiment, the intermediary microorganism can be an engineered bacteria, virus, or phage that contains the composition, system(s) and/or component(s) thereof and/or vectors and/or vector systems. The method can include administering an intermediary microorganism containing the composition, system(s) and/or component(s) thereof and/or vectors and/or vector systems to the subject to be treated. The intermediary microorganism can then produce the compositions and/or component thereof or transfer a composition, system, polynucleotide to the pathogenic organism. In embodiments, where the compositions and/or component thereof, vector, or vector system is transferred to the pathogenic microorganism, the composition, system, or component thereof is then produced in the pathogenic microorganism and modifies the pathogenic microorganism such that it is less virulent, killed, inhibited, or is otherwise rendered incapable of causing disease and/or infecting and/or replicating in a host or cell thereof.

In one embodiment, where the pathogenic microorganism inserts its genetic material into the host cell's genome (e.g., a virus), the composition, system, can be designed such that it modifies the host cell's genome such that the viral DNA or cDNA cannot be replicated by the host cell's machinery into a functional virus. In one embodiment, where the pathogenic microorganism inserts its genetic material into the host cell's genome (e.g., a virus), the composition, system, can be designed such that it modifies the host cell's genome such that the viral DNA or cDNA is deleted from the host cell's genome.

It will be appreciated that inhibiting or killing the pathogenic microorganism, the disease and/or condition that its infection causes in the subject can be treated or prevented. Thus, also provided herein are methods of treating and/or preventing one or more diseases or symptoms thereof caused by any one or more pathogenic microorganisms, such as any of those described herein.

### Mitochondrial Diseases

Some of the most challenging mitochondrial disorders arise from mutations in mitochondrial DNA (mtDNA), a high copy number genome that is maternally inherited. In one embodiment, mtDNA mutations can be modified using a composition, system, described herein. In one embodiment, the mitochondrial disease that can be diagnosed, prognosed, treated, and/or prevented can be MELAS (mitochondrial myopathy encephalopathy, and lactic acidosis and stroke-like episodes), CPEO/PEO (chronic progressive external ophthalmoplegia syndrome/progressive external ophthalmoplegia), KSS (Kearns-Sayre syndrome), MIDD (maternally inherited diabetes and deafness), MERRF (myoclonic epilepsy associated with ragged red fibers), NIDDM (noninsulin-dependent diabetes mellitus), LHON (Leber hereditary optic neuropathy), LS (Leigh Syndrome) an aminoglycoside induced hearing disorder, NARP (neuropathy, ataxia, and pigmentary retinopathy), Extrapyramidal disorder with akinesia-rigidity, psychosis and SNHL, Nonsyndromic hearing loss a cardiomyopathy, an encephalomyopathy, Pearson's syndrome, or a combination thereof.

In one embodiment, the mtDNA of a subject can be modified in vivo or ex vivo. In one embodiment, where the mtDNA is modified ex vivo, after modification the cells containing the modified mitochondria can be administered back to the subject. In one embodiment, the composition, system, or component thereof can be capable of correcting an mtDNA mutation, or a combination thereof.

In one embodiment, at least one of the one or more mtDNA mutations is selected from the group consisting of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem (SEQ ID NO: 235) repeats at positions 305-314 and/or 956-965, deletion at positions from 8,469-13,447, 4,308-14,874, and/or 4,398-14,822, 961ins/delC, the mitochondrial common deletion (e.g., mtDNA 4,977 bp deletion), and combinations thereof.

In one embodiment, the mitochondrial mutation can be any mutation as set forth in or as identified by use of one or more bioinformatic tools available at Mitomap available at mitomap.org. Such tools include, but are not limited to, “Variant Search, aka Market Finder”, Find Sequences for Any Haplogroup, aka “Sequence Finder”, “Variant Info”, “POLG Pathogenicity Prediction Server”, “MITOMASTER”, “Allele Search”, “Sequence and Variant Downloads”, “Data Downloads”. MitoMap contains reports of mutations in mtDNA that can be associated with disease and maintains a database of reported mitochondrial DNA Base Substitution Diseases: rRNA/tRNA mutations.

In one embodiment, the method includes delivering a composition, system, and/or a component thereof to a cell, and more specifically one or more mitochondria in a cell, allowing the composition, system, and/or component thereof to modify one or more target polynucleotides in the cell, and more specifically one or more mitochondria in the cell. The target polynucleotides can correspond to a mutation in the mtDNA, such as any one or more of those described herein. In one embodiment, the modification can alter a function of the mitochondria such that the mitochondria functions normally or at least is/are less dysfunctional as compared to an unmodified mitochondria. Modification can occur in vivo or ex vivo. Where modification is performed ex vivo, cells containing modified mitochondria can be administered to a subject in need thereof in an autologous or allogenic manner.

### Microbiome Modification

Microbiomes play important roles in health and disease. For example, the gut microbiome can play a role in health by controlling digestion, preventing growth of pathogenic microorganisms and have been suggested to influence mood and emotion. Imbalanced microbiomes can promote disease and are suggested to contribute to weight gain, unregulated blood sugar, high cholesterol, cancer, and other disorders. A healthy microbiome has a series of joint characteristics that can be distinguished from non-healthy individuals, thus detection and identification of the disease-associated microbiome can be used to diagnose and detect disease in an individual. The compositions, systems, and components thereof can be used to screen the microbiome cell population and be used to identify a disease associated microbiome. Cell screening methods utilizing compositions, systems, and components thereof are described elsewhere herein and can be applied to screening a microbiome, such as a gut, skin, vagina, and/or oral microbiome, of a subject.

In one embodiment, the microbe population of a microbiome in a subject can be modified using a composition, system, and/or component thereof described herein. In one embodiment, the composition, system, and/or component thereof can be used to identify and select one or more cell types in the microbiome and remove them from the microbiome population. Exemplary methods of selecting cells using a composition, system, and/or component thereof are described elsewhere herein. In this way, the make-up or microorganism profile of the microbiome can be altered. In one embodiment, the alteration causes a change from a diseased microbiome composition to a healthy microbiome composition. In this way, the ratio of one type or species of microorganism to another can be modified, such as going from a diseased ratio to a healthy ratio. In one embodiment, the cells selected are pathogenic microorganisms.

In one embodiment, the compositions and systems described herein can be used to modify a polynucleotide in a microorganism of a microbiome in a subject. In one embodiment, the microorganism is a pathogenic microorganism. In one embodiment, the microorganism is a commensal and non-pathogenic microorganism. Methods of modifying polynucleotides in a cell in the subject are described elsewhere herein and can be applied to these embodiments.

### Models of Diseases and Conditions

In an aspect, the invention provides a method of modeling a disease associated with a genomic locus in a eukaryotic organism or a non-human organism comprising manipulation of a target sequence within a coding, non-coding or regulatory element of said genomic locus comprising delivering a non-naturally occurring or engineered composition comprising a viral vector system comprising one or more viral vectors operably encoding a composition for expression thereof, wherein the composition comprises particle delivery system or the delivery system or the virus particle of any one of the above embodiments or the cell of any one of the above embodiment.

In one aspect, the invention provides a method of generating a model eukaryotic cell that can include one or more a mutated disease genes and/or infectious microorganisms. In one embodiment, a disease gene is any gene associated an increase in the risk of having or developing a disease. In one embodiment, the method includes (a) introducing one or more vectors into a eukaryotic cell, wherein the one or more vectors comprise a composition, system, and/or component thereof and/or a vector or vector system that is capable of driving expression of a composition, system, and/or component thereof including, but not limited to: a nucleic acid component molecule sequence, one or more TnpB polypeptides, and combinations thereof and (b) allowing a composition, system, or complex to bind to one or more target polynucleotides, e.g., to effect cleavage, nicking, or other modification of the target polynucleotide within said disease gene, wherein the composition, system, or complex is composed of one or more TnpB polypeptide complexed with (1) one or more nucleic acid component molecule sequences that is/are hybridized to the target sequence(s) within the target polynucleotide(s), and optionally (2) the nucleic acid component scaffold sequence(s), thereby generating a model eukaryotic cell comprising one or more mutated disease gene(s). Thus, in one embodiment the composition and system, contains nucleic acid molecules for and drives expression of one or more of: a TnpB polypeptide, a nucleic acid component molecule sequence and/or a Homologous Recombination template and/or a stabilizing ligand if the TnpB polypeptide has a destabilization domain. In one embodiment, said cleavage comprises cleaving one or two strands at the location of the target sequence by the TnpB polypeptide. In one embodiment, nicking comprises nicking one or two strands at the location of the target sequence by the TnpB polypeptide. In one embodiment, said cleavage or nicking results in modified transcription of a target polynucleotide. In one embodiment, modification results in decreased transcription of the target polynucleotide. In one embodiment, the method further comprises repairing said cleaved or nicked target polynucleotide by homologous recombination with an recombination template polynucleotide, wherein said repair results in a mutation comprising an insertion, deletion, or substitution of one or more nucleotides of said target polynucleotide. In one embodiment, said mutation results in one or more amino acid changes in a protein expression from a gene comprising the target sequence.

The disease modeled can be any disease with a genetic or epigenetic component. In one embodiment, the disease modeled can be any as discussed elsewhere herein.

### In Situ Disease Detection

The compositions, systems, and/or components thereof can be used for diagnostic methods of detection such as in CASFISH (see e.g., Deng et al. 2015. PNAS USA 112(38): 11870-11875), CRISPR-Live FISH (see e.g., Wang et al. 2020. Science; 365(6459): 1301-1305), sm-FISH (Lee and Jefcoate. 2017. Front. Endocrinol. doi.org/10.3389/fendo.2017.00289), sequential FISH CRISPRainbow (Ma et al. Nat Biotechnol, 34(2016), pp. 528-530), CRISPR-Sirius (Nat Methods, 15(2018), pp. 928-931), Casilio (Cheng et al. Cell Res, 26(2016), pp. 254-257), Halo-Tag based genomic loci visualization techniques (e.g., Deng et al. 2015. PNAS USA 112(38): 11870-11875; Knight et al., Science, 350(2015), pp. 823-826), RNA-aptamer based methods (e.g., Ma et al., J Cell Biol, 214(2016), pp. 529-537), molecular beacon-based methods (e.g., Zhao et al. Biomaterials, 100(2016), pp. 172-183; Wu et al. Nucleic Acids Res (2018)), Quantum Dot-based systems (e.g., Ma et al. Anal Chem, 89(2017), pp. 12896-12901), multiplexed methods (e.g., Ma et al., Proc Natl Acad Sci USA, 112(2015), pp. 3002-3007; Fu et al. Nat Commun, 7(2016), p. 11707; Ma et al. Nat Biotechnol, 34(2016), pp. 528-530; Shao et al. Nucleic Acids Res, 44(2016), Article e86); Wang et al. Sci Rep, 6(2016), p. 26857), and other in situ CRISPR-hybridization based methods (e.g., Chen et al. Cell, 155(2013), pp. 1479-1491; Gu et al. Science, 359(2018), pp. 1050-1055; Tanebaum et al. Cell, 159(2014), pp. 635-646; Ye et al. Protein Cell, 8(2017), pp. 853-855; Chen et al. Nat Commun, 9(2018), p. 5065; Shao et al. ACS Synth Biol (2017); Fu et al. Nat Commun, 7(2016), p. 11707; Shao et al. Nucleic Acids Res, 44(2016), Article e86; Wang et al., Sci Rep, 6(2016), p. 26857), all of which are incorporated by reference herein as if expressed in their entirety and whose teachings can be adapted to the compositions, systems, and components thereof described herein in view of the description herein.

In one embodiment, the composition, system, or component thereof can be used in a detection method, such as an in situ detection method described herein. In one embodiment, the composition, system, or component thereof can include a catalytically inactive TnpB polypeptide described herein and use this system in detection methods such as fluorescence in situ hybridization (FISH) or any other described herein. In one embodiment, the inactivated TnpB polypeptide, which lacks the ability to produce DNA double-strand breaks may be fused with a marker, such as fluorescent protein, such as the enhanced green fluorescent protein (eEGFP) and co-expressed with small nucleic acid component molecules to target pericentric, centric and telomeric repeats in vivo. The dead TnpB polypeptide or system thereof can be used to visualize both repetitive sequences and individual genes in the human genome. Such new applications of labelled dead TnpB polypeptide and compositions, systems, thereof can be important in imaging cells and studying the functional nuclear architecture, especially in cases with a small nucleus volume or complex 3-D structures.

### Cell Selection

In one embodiment, the compositions, systems, and/or components thereof described herein can be used in a method to screen and/or select cells. In one embodiment, composition, system-based screening/selection method can be used to identify diseased cells in a cell population. In one embodiment, selection of the cells results in a modification in the cells such that the selected cells die. In this way, diseased cells can be identified, and removed from the healthy cell population. In one embodiment, the diseased cells can be a cancer cell, pre-cancerous cell, a virus or other pathogenic organism infected cells, or otherwise abnormal cell. In one embodiment, the modification can impart another detectable change in the cells to be selected (e.g., a functional change and/or genomic barcode) that facilitates selection of the desired cells. In one embodiment a negative selection scheme can be used to obtain a desired cell population. In these embodiments, the cells to be selected against are modified, thus can be removed from the cell population based on their death or identification or sorting based the detectable change imparted on the cells. Thus, in these embodiments, the remaining cells after selection are the desired cell population.

In one embodiment, a method of selecting one or more cell(s) containing a polynucleotide modification can include: introducing one or more composition, system, (s) and/or components thereof, and/or vectors or vector systems into the cell(s), wherein the composition, system, (s) and/or components thereof, and/or vectors or vector systems contains and/or is capable of expressing one or more of: a TnpB polypeptide, an nucleic acid component sequence, and an recombination template; wherein, for example that which is being expressed is within and expressed in vivo by the composition, system, vector or vector system and/or the recombination template comprises the one or more mutations that abolish TnpB polypeptide cleavage; allowing homologous recombination of the recombination template with the target polynucleotide in the cell(s) to be selected; allowing a composition, system, or complex to bind to a target polynucleotide to effect cleavage of the target polynucleotide within said gene, wherein the AAV-complex comprises the TnpB polypeptide complexed with (1) the nucleic acid component molecule sequence that is hybridized to the target sequence within the target polynucleotide, and (2) the nucleic acid component scaffold, wherein binding of the complex to the target polynucleotide induces cell death or imparts some other detectable change to the cell, thereby allowing one or more cell(s) in which one or more mutations have been introduced to be selected. In one embodiment, the cell to be selected may be a eukaryotic cell. In one embodiment, the cell to be selected may be a prokaryotic cell. Selection of specific cells via the methods herein can be performed without requiring a selection marker or a two-step process that may include a counter-selection system.

### Therapeutic Agent Development

The compositions, systems, and components thereof described herein can be used to develop TnpB polypeptide-based biologically active agents, such as small molecule therapeutics. Thus, described herein are methods for developing a biologically active agent that modulates a cell function and/or signaling event associated with a disease and/or disease gene. In one embodiment, the method comprises (a) contacting a test compound with a diseased cell and/or a cell containing a disease gene cell; and (b) detecting a change in a readout that is indicative of a reduction or an augmentation of a cell signaling event or other cell functionality associated with said disease or disease gene, thereby developing said biologically active agent that modulates said cell signaling event or other functionality associated with said disease gene. In one embodiment, the diseased cell is a model cell described elsewhere herein. In one embodiment, the diseased cell is a diseased cell isolated from a subject in need of treatment. In one embodiment, the test compound is a small molecule agent. In one embodiment, test compound is a small molecule agent. In one embodiment, the test compound is a biologic molecule agent.

In one embodiment, the method involves developing a therapeutic based on the composition, system, described herein. In particular embodiments, the therapeutic comprises a TnpB polypeptide and/or a nucleic acid component with a reprogrammable spacer capable of hybridizing to a target sequence of interest. In particular embodiments, the therapeutic is a vector or vector system that can contain a) a first regulatory element operably linked to a nucleotide sequence encoding the TnpB polypeptide; and b) a second regulatory element operably linked to one or more nucleotide sequences encoding one or more nucleic acid molecules comprising a nucleic acid component comprising a reprogrammable spacer sequence, a conserved RNA sequence; wherein components (a) and (b) are located on same or different vectors. In particular embodiments, the biologically active agent is a composition comprising a delivery system operably configured to deliver composition, system, or components thereof, and/or or one or more polynucleotide sequences, vectors, or vector systems containing or encoding said components into a cell and capable of forming a complex with the components of the composition and system herein, and wherein said complex is operable in the cell. In one embodiment, the complex can include the TnpB polypeptide as described herein, nucleic acid component scaffold comprising the guide sequence (reprogrammable spacer sequence), and a conserved nucleotide sequence. In any such compositions, the delivery system can be a yeast system, a lipofection system, a microinjection system, a biolistic system, virosomes, liposomes, immunoliposomes, polycations, lipid: nucleic acid conjugates or artificial virions, or any other system as described herein. In particular embodiments, the delivery is via a particle, a nanoparticle, a lipid or a cell penetrating peptide (CPP).

Also described herein are methods for developing or designing a composition, system, optionally a composition, system, based therapy or therapeutic, comprising (a) selecting for a (therapeutic) locus of interest nucleic acid component target sites, wherein said target sites have minimal sequence variation across a population, and from said selected target sites subselecting target sites, wherein a nucleic acid component directed against said target sites recognizes a minimal number of off-target sites across said population, or (b) selecting for a (therapeutic) locus of interest nucleic acid component target sites, wherein said target sites have minimal sequence variation across a population, or selecting for a (therapeutic) locus of interest nucleic acid component target sites, wherein a nucleic acid component directed against said target sites recognizes a minimal number of off-target sites across said population, and optionally estimating the number of (sub) selected target sites needed to treat or otherwise modulate or manipulate a population, and optionally validating one or more of the (sub) selected target sites for an individual subject, optionally designing one or more nucleic acid component recognizing one or more of said (sub) selected target sites.

In one embodiment, the method for developing or designing a nucleic acid component for use in a composition, system, optionally a composition, system, based therapy or therapeutic, can include (a) selecting for a (therapeutic) locus of interest nucleic acid component target sites, wherein said target sites have minimal sequence variation across a population, and from said selected target sites subselecting target sites, wherein a nucleic acid component molecule directed against said target sites recognizes a minimal number of off-target sites across said population, or (b) selecting for a (therapeutic) locus of interest nucleic acid component molecule target sites, wherein said target sites have minimal sequence variation across a population, or selecting for a (therapeutic) locus of interest nucleic acid component molecule target sites, wherein a nucleic acid component molecule directed against said target sites recognizes a minimal number of off-target sites across said population, and optionally estimating the number of (sub) selected target sites needed to treat or otherwise modulate or manipulate a population, optionally validating one or more of the (sub) selected target sites for an individual subject, optionally designing one or more nucleic acid component molecule recognizing one or more of said (sub) selected target sites.

In one embodiment, thee method for developing or designing a composition, system, optionally a composition, system, based therapy or therapeutic in a population, can include (a) selecting for a (therapeutic) locus of interest reprogrammable spacer target sites, wherein said target sites have minimal sequence variation across a population, and from said selected target sites subselecting target sites, wherein a nucleic acid component directed against said target sites recognizes a minimal number of off-target sites across said population, or (b) selecting for a (therapeutic) locus of interest nucleic acid component reprogrammable spacer target sites, wherein said target sites have minimal sequence variation across a population, or selecting for a (therapeutic) locus of interest nucleic acid component reprogrammable spacer target sites, wherein a nucleic acid component directed against said target sites recognizes a minimal number of off-target sites across said population, and optionally estimating the number of (sub) selected target sites needed to treat or otherwise modulate or manipulate a population, optionally validating one or more of the (sub) selected target sites for an individual subject, optionally designing one or more nucleic acid component recognizing one or more of said (sub) selected target sites.

In one embodiment the method for developing or designing a nucleic acid component molecule for use in a composition, system, optionally a composition, system, based therapy or therapeutic in a population, can include (a) selecting for a (therapeutic) locus of interest nucleic acid component molecule target sites, wherein said target sites have minimal sequence variation across a population, and from said selected target sites subselecting target sites, wherein a nucleic acid component molecule directed against said target sites recognizes a minimal number of off-target sites across said population, or (b) selecting for a (therapeutic) locus of interest nucleic acid component molecule target sites, wherein said target sites have minimal sequence variation across a population, or selecting for a (therapeutic) locus of interest nucleic acid component molecule target sites, wherein a nucleic acid component molecule directed against said target sites recognizes a minimal number of off-target sites across said population, and optionally estimating the number of (sub) selected target sites needed to treat or otherwise modulate or manipulate a population, optionally validating one or more of the (sub) selected target sites for an individual subject, optionally designing one or more nucleic acid component reprogrammable spacer recognizing one or more of said (sub) selected target sites.

In one embodiment, the method for developing or designing a composition, system, such as a composition, system, based therapy or therapeutic, optionally in a population; or for developing or designing a nucleic acid component reprogrammable spacer for use in a composition, system, optionally a composition, system, based therapy or therapeutic, optionally in a population, can include selecting a set of target sequences for one or more loci in a target population, wherein the target sequences do not contain variants occurring above a threshold allele frequency in the target population (i.e. platinum target sequences); removing from said selected (platinum) target sequences any target sequences having high frequency off-target candidates (relative to other (platinum) targets in the set) to define a final target sequence set; preparing one or more, such as a set of compositions, systems, based on the final target sequence set, optionally wherein a number of composition prepared is based (at least in part) on the size of a target population.

In one embodiment, off-target candidates/off-targets, TAMrestrictiveness, target cleavage efficiency, or effector protein specificity is identified or determined using a sequencing-based double-strand break (DSB) detection assay, such as described herein elsewhere. In one embodiment, off-target candidates/off-targets are identified or determined using a sequencing-based double-strand break (DSB) detection assay, such as described herein elsewhere. In one embodiment, off-targets, or off target candidates have at least 1, preferably 1-3, mismatches or (distal) TAMmismatches, such as 1 or more, such as 1, 2, 3, or more (distal) TAMmismatches. In one embodiment, sequencing-based DSB detection assay comprises labeling a site of a DSB with an adapter comprising a primer binding site, labeling a site of a DSB with a barcode or unique molecular identifier, or combination thereof, as described herein elsewhere.

It will be understood that the reprogrammable spacer sequence of the nucleic acid component is 100% complementary to the target site, i.e., does not comprise any mismatch with the target site. It will be further understood that “recognition” of an (off-) target site by a reprogrammable spacer presupposes composition, system, functionality, i.e., an (off-) target site is only recognized by a reprogrammable spacer RNA if binding of the reprogrammable spacer RNA to the (off-) target site leads to composition, system, activity (such as induction of single or double strand DNA cleavage, transcriptional modulation, etc.).

In one embodiment, the target sites having minimal sequence variation across a population are characterized by absence of sequence variation in at least 99%, preferably at least 99.9%, more preferably at least 99.99% of the population. In one embodiment, optimizing target location comprises selecting target sequences or loci having an absence of sequence variation in at least 99%, %, preferably at least 99.9%, more preferably at least 99.99% of a population. These targets are referred to herein elsewhere also as “platinum targets”. In one embodiment, said population comprises at least 1000 individuals, such as at least 5000 individuals, such as at least 10000 individuals, such as at least 50000 individuals.

In one embodiment, the off-target sites are characterized by at least one mismatch between the off-target site and the nucleic acid component. In one embodiment, the off-target sites are characterized by at most five, preferably at most four, more preferably at most three mismatches between the off-target site and the nucleic acid component. In one embodiment, the off-target sites are characterized by at least one mismatch between the off-target site and the nucleic acid component and by at most five, preferably at most four, more preferably at most three mismatches between the off-target site and the nucleic acid component.

In one embodiment, said minimal number of off-target sites across said population is determined for high-frequency haplotypes in said population. In one embodiment, said minimal number of off-target sites across said population is determined for high-frequency haplotypes of the off-target site locus in said population. In one embodiment, said minimal number of off-target sites across said population is determined for high-frequency haplotypes of the target site locus in said population. In one embodiment, the high-frequency haplotypes are characterized by occurrence in at least 0.1% of the population.

In one embodiment, the number of (sub) selected target sites needed to treat a population is estimated based on based low frequency sequence variation, such as low frequency sequence variation captured in large scale sequencing datasets. In one embodiment, the number of (sub) selected target sites needed to treat a population of a given size is estimated.

In one embodiment, the method further comprises obtaining genome sequencing data of a subject to be treated; and treating the subject with a composition, system, selected from the set of compositions, systems, wherein the composition, system, selected is based (at least in part) on the genome sequencing data of the individual. In one embodiment, the ((sub) selected) target is validated by genome sequencing, preferably whole genome sequencing.

In one embodiment, target sequences or loci as described herein are (further) selected based on optimization of one or more parameters, such as TAMtype (natural or modified), TAMnucleotide content, TAMlength, target sequence length, TAMrestrictiveness, target cleavage efficiency, and target sequence position within a gene, a locus or other genomic region. Methods of optimization are discussed in greater detail elsewhere herein.

In one embodiment, target sequences or loci as described herein are (further) selected based on optimization of one or more of target loci location, target length, target specificity, and TAMcharacteristics. As used herein, TAMcharacteristics may comprise for instance TAMsequence, TAMlength, and/or TAM GC contents. In one embodiment, optimizing TAMcharacteristics comprises optimizing nucleotide content of aTAM. In one embodiment, optimizing nucleotide content of TAMis selecting a TAMwith a motif that maximizes abundance in the one or more target loci, minimizes mutation frequency, or both. Minimizing mutation frequency can for instance be achieved by selecting TAMsequences devoid of or having low or minimal CpG.

In one embodiment, the effector protein for each composition and system, in the set of compositions, systems, is selected based on optimization of one or more parameters selected from the group consisting of; effector protein size, ability of effector protein to access regions of high chromatin accessibility, degree of uniform enzyme activity across genomic targets, epigenetic tolerance, mismatch/budge tolerance, effector protein specificity, effector protein stability or half-life, effector protein immunogenicity or toxicity. Methods of optimization are discussed in greater detail elsewhere herein.

### Optimization of the Systems

The methods of the present invention can involve optimization of selected parameters or variables associated with the composition, system, and/or its functionality, as described herein further elsewhere. Optimization of the composition, system, in the methods as described herein may depend on the target(s), such as the therapeutic target or therapeutic targets, the mode or type of composition, system, modulation, such as composition, system, based therapeutic target(s) modulation, modification, or manipulation, as well as the delivery of the composition, system, components. One or more targets may be selected, depending on the genotypic and/or phenotypic outcome. For instance, one or more therapeutic targets may be selected, depending on (genetic) disease etiology or the desired therapeutic outcome. The (therapeutic) target(s) may be a single gene, locus, or other genomic site, or may be multiple genes, loci or other genomic sites. As is known in the art, a single gene, locus, or other genomic site may be targeted more than once, such as by use of multiple nucleic acid components, or nucleic acid component scaffold and multiple reprogrammable spacers.

The activity of the composition and/or system, such as TnpB polypeptide-based therapy or therapeutics may involve target disruption, such as target mutation, such as leading to gene knockout. The activity of the composition and/or system, such as TnpB polypeptide-based therapy or therapeutics may involve replacement of particular target sites, such as leading to target correction. TnpB polypeptide based therapy or therapeutics may involve removal of particular target sites, such as leading to target deletion. The activity of the composition and/or system, such as TnpB polypeptide-based therapy or therapeutics may involve modulation of target site functionality, such as target site activity or accessibility, leading for instance to (transcriptional and/or epigenetic) gene or genomic region activation or gene or genomic region silencing. The skilled person will understand that modulation of target site functionality may involve TnpB polypeptide mutation (such as for instance generation of a catalytically inactive TnpB polypeptide) and/or functionalization (such as for instance fusion of the TnpB polypeptide with a heterologous functional domain, such as a transcriptional activator or repressor), as described herein elsewhere.

Accordingly, in an aspect, the invention relates to a method as described herein, comprising selection of one or more (therapeutic) target, selecting one or more functionality of the composition and/or system, and optimization of selected parameters or variables associated with the composition and/or its functionality. In a related aspect, the invention relates to a method as described herein, comprising (a) selecting one or more (therapeutic) target loci, (b) selecting one or more composition functionalities, (c) optionally selecting one or more modes of delivery, and preparing, developing, or designing a composition herein selected based on steps (a)-(c).

In one embodiment, the functionality of the composition and/or system comprises genomic mutation. In one embodiment, the functionality of the composition and/or system comprises single genomic mutation. In one embodiment, the functionality of the composition and/or system functionality comprises multiple genomic mutation. In one embodiment, the functionality of the composition and/or system comprises gene knockout. In one embodiment, the functionality of the composition and/or system comprises single gene knockout. In one embodiment, the functionality of the composition and/or system comprises multiple gene knockout. In one embodiment, the functionality of the composition and/or system comprises gene correction. In one embodiment, the functionality of the composition and/or system comprises single gene correction. In one embodiment, the functionality of the composition and/or system comprises multiple gene correction. In one embodiment, the functionality of the composition and/or system comprises genomic region correction. In one embodiment, the functionality of the composition and/or system comprises single genomic region correction. In one embodiment, the functionality of the composition and/or system comprises multiple genomic region correction. In one embodiment, the functionality of the composition and/or system comprises gene deletion. In one embodiment, the functionality of the composition and/or system comprises single gene deletion. In one embodiment, the functionality of the composition and/or system comprises multiple gene deletion. In one embodiment, the functionality of the composition and/or system comprises genomic region deletion. In one embodiment, the functionality of the composition and/or system comprises single genomic region deletion. In one embodiment, the functionality of the composition and/or system comprises multiple genomic region deletion. In one embodiment, the functionality of the composition and/or system comprises modulation of gene or genomic region functionality. In one embodiment, the functionality of the composition and/or system comprises modulation of single gene or genomic region functionality. In one embodiment, the functionality of the composition and/or system comprises modulation of multiple gene or genomic region functionality. In one embodiment, the functionality of the composition and/or system comprises gene or genomic region functionality, such as gene or genomic region activity. In one embodiment, the functionality of the composition and/or system comprises single gene or genomic region functionality, such as gene or genomic region activity. In one embodiment, the functionality of the composition and/or system comprises multiple gene or genomic region functionality, such as gene or genomic region activity. In one embodiment, the functionality of the composition and/or system comprises modulation gene activity or accessibility optionally leading to transcriptional and/or epigenetic gene or genomic region activation or gene or genomic region silencing. In one embodiment, the functionality of the composition and/or system comprises modulation single gene activity or accessibility optionally leading to transcriptional and/or epigenetic gene or genomic region activation or gene or genomic region silencing. In one embodiment, the functionality of the composition and/or system comprises modulation multiple gene activity or accessibility optionally leading to transcriptional and/or epigenetic gene or genomic region activation or gene or genomic region silencing.

Optimization of selected parameters or variables in the methods as described herein may result in optimized or improved the system, such as TnpB polypeptide-based therapy or therapeutic, specificity, efficacy, and/or safety. In one embodiment, one or more of the following parameters or variables are taken into account, are selected, or are optimized in the methods of the invention as described herein: TnpB polypeptide allosteric interactions, TnpB polypeptide functional domains and functional domain interactions, TnpB polypeptide specificity, nucleic acid component specificity, composition specificity, TAMrestrictiveness, TAMtype (natural or modified), TAMnucleotide content, TAMlength, TnpB polypeptide activity, nucleic acid component activity, TnpB polypeptide/nucleic acid component molecule complex activity, target cleavage efficiency, target site selection, target sequence length, ability of effector protein to access regions of high chromatin accessibility, degree of uniform enzyme activity across genomic targets, epigenetic tolerance, mismatch/budge tolerance, TnpB polypeptide stability, TnpB polypeptide mRNA stability, nucleic acid component molecule stability, TnpB polypeptide complex stability, TnpB polypeptide protein or mRNA immunogenicity or toxicity, nucleic acid component molecule immunogenicity or toxicity, TnpB polypeptide immunogenicity or toxicity, TnpB polypeptide or mRNA dose or titer, nucleic acid component molecule dose or titer, dose or titer, TnpB polypeptide protein size, TnpB polypeptide expression level, nucleic acid component molecule expression level, TnpB polypeptide expression level, TnpB polypeptide spatiotemporal expression, nucleic acid component molecule spatiotemporal expression, TnpB polypeptide/nucleic acid component spatiotemporal expression.

By means of example, and without limitation, parameter or variable optimization may be achieved as follows. TnpB polypeptide specificity may be optimized by selecting the most specific TnpB polypeptide, e.g., TnpB. This may be achieved for instance by selecting the most specific TnpB polypeptide orthologue or by specific TnpB polypeptide mutations which increase specificity. nucleic acid component specificity may be optimized by selecting the most specific nucleic acid component. This can be achieved for instance by selecting nucleic acid component having low homology, i.e., at least one or preferably more, such as at least 2, or preferably at least 3, mismatches to off-target sites. The specificity may be optimized by increasing TnpB polypeptide specificity and/or nucleic acid component specificity as above.

Target length or target sequence length may be optimized, for instance, by selecting the appropriate TnpB polypeptide, such as the appropriate TnpB polypeptide recognizing a desired target or target sequence nucleotide length. Alternatively, or in addition, the target (sequence) length may be optimized by providing a target having a length deviating from the target (sequence) length typically associated with the TnpB polypeptide, such as the naturally occurring TnpB polypeptide. The TnpB polypeptide or target (sequence) length may be naturally occurring or may for instance be optimized based on TnpB polypeptide mutants having an altered target (sequence) length recognition, or target (sequence) length recognition repertoire. For instance, increasing or decreasing target (sequence) length may influence target recognition and/or off-target recognition. TnpB polypeptide activity may be optimized by selecting the most active TnpB polypeptide. This may be achieved for instance by selecting the most active TnpB polypeptide ortholog or by specific TnpB polypeptide mutations which increase activity. The ability of the TnpB polypeptide protein to access regions of high chromatin accessibility, may be optimized by selecting the appropriate TnpB polypeptide or mutant thereof, and can consider the size of the TnpB polypeptide, charge, or other dimensional variables etc. The degree of uniform TnpB polypeptide activity may be optimized by selecting the appropriate TnpB polypeptide or mutant thereof, and can consider TnpB polypeptide specificity and/or activity, TAMspecificity, target length, mismatch tolerance, epigenetic tolerance, TnpB polypeptide and/or nucleic acid component stability and/or half-life, TnpB polypeptide and/or nucleic acid component immunogenicity and/or toxicity, etc. nucleic acid component activity may be optimized by selecting the most active nucleic acid component. In one embodiment, this can be achieved by increasing nucleic acid component stability through RNA modification. compositions activity may be optimized by increasing TnpB polypeptide activity and/or nucleic acid component activity as above.

The target site selection may be optimized by selecting the optimal position of the target site within a gene, locus or other genomic region. The target site selection may be optimized by optimizing target location comprises selecting a target sequence with a gene, locus, or other genomic region having low variability. This may be achieved for instance by selecting a target site in an early and/or conserved exon or domain (i.e., having low variability, such as polymorphisms, within a population).

In one embodiment, optimizing target (sequence) length comprises selecting a target sequence within one or more target loci between 5 and 25 nucleotides. In one embodiment, a target sequence is 20 nucleotides.

In one embodiment, optimizing target specificity comprises selecting targets loci that minimize off-target candidates.

In one embodiment, the target site may be selected by minimization of off-target effects (e.g., off-targets qualified as having 1-5, 1-4, or preferably 1-3 mismatches compared to target, preferably also considering variability within a population. TnpB polypeptide stability may be optimized by selecting TnpB polypeptide having appropriate half-life, such as preferably a short half-life while still capable of maintaining sufficient activity. In one embodiment, this can be achieved by selecting an appropriate TnpB polypeptide orthologue having a specific half-life or by specific TnpB polypeptide mutations or modifications which affect half-life or stability, such as inclusion (e.g., fusion) of stabilizing or destabilizing domains or sequences. TnpB polypeptide mRNA stability may be optimized by increasing or decreasing TnpB polypeptide mRNA stability. In one embodiment, this can be achieved by increasing or TnpB polypeptide mRNA stability through mRNA modification. nucleic acid component stability may be optimized by increasing or decreasing nucleic acid component stability. In one embodiment, this can be achieved by increasing or decreasing nucleic acid component stability through RNA modification. The stability may be optimized by increasing or decreasing TnpB polypeptide stability and/or nucleic acid component molecule stability as above. TnpB polypeptide protein or mRNA immunogenicity or toxicity may be optimized by decreasing TnpB polypeptide or mRNA immunogenicity or toxicity. In one embodiment, this can be achieved by mRNA or protein modifications. Similarly, in case of DNA based expression systems, DNA immunogenicity or toxicity may be decreased. nucleic acid component immunogenicity or toxicity may be optimized by decreasing nucleic acid component immunogenicity or toxicity. In one embodiment, this can be achieved by nucleic acid component modifications. Similarly, in case of DNA based expression systems, DNA immunogenicity or toxicity may be decreased. The immunogenicity or toxicity may be optimized by decreasing TnpB polypeptide immunogenicity or toxicity and/or nucleic acid component immunogenicity or toxicity as above, or by selecting the least immunogenic or toxic TnpB polypeptide/nucleic acid component combination. Similarly, in case of DNA based expression systems, DNA immunogenicity or toxicity may be decreased. TnpB polypeptide protein or mRNA dose or titer may be optimized by selecting dosage or titer to minimize toxicity and/or maximize specificity and/or efficacy. nucleic acid component dose or titer may be optimized by selecting dosage or titer to minimize toxicity and/or maximize specificity and/or efficacy. The composition dose or titer may be optimized by selecting dosage or titer to minimize toxicity and/or maximize specificity and/or efficacy. The TnpB polypeptide size may be optimized by selecting minimal protein size to increase efficiency of delivery, in particular for virus mediated delivery. TnpB polypeptide, nucleic acid component, or complex thereof expression level may be optimized by limiting (or extending) the duration of expression and/or limiting (or increasing) expression level. This may be achieved for instance by using self-inactivating compositions, systems, such as including a self-targeting (e.g., TnpB polypeptide targeting) nucleic acid component molecule, by using viral vectors having limited expression duration, by using appropriate promoters for low (or high) expression levels, by combining different delivery methods for individual TnpB system components, such as virus mediated delivery of TnpB polypeptide encoding nucleic acid combined with non-virus mediated delivery of nucleic acid component, or virus mediated delivery of nucleic acid component combined with non-virus mediated delivery of TnpB polypeptide or mRNA. TnpB polypeptide, nucleic acid component, or TnpB complex spatiotemporal expression may be optimized by appropriate choice of conditional and/or inducible expression systems, including controllable TnpB polypeptide activity optionally a destabilized TnpB polypeptide and/or a split TnpB polypeptide, and/or cell- or tissue-specific expression systems.

In an aspect, the invention relates to a method as described herein, comprising selection of one or more (therapeutic) target, selecting the functionality of the composition and/or system, selecting composition mode of delivery, selecting composition delivery vehicle or expression system, and optimization of selected parameters or variables associated with the composition and/or its functionality, optionally wherein the parameters or variables are one or more selected from TnpB polypeptide specificity, nucleic acid component specificity, TnpB complex specificity, TnpB polypeptide activity, nucleic acid component molecule activity, TnpB polypeptide/nucleic acid component complex activity, target cleavage efficiency, target site selection, target sequence length, ability of effector protein to access regions of high chromatin accessibility, degree of uniform enzyme activity across genomic targets, epigenetic tolerance, mismatch/budge tolerance, TnpB polypeptide stability, TnpB polypeptide mRNA stability, nucleic acid component stability, TnpB complex stability, TnpB polypeptide protein or mRNA immunogenicity or toxicity, nucleic acid component immunogenicity or toxicity, TnpB polypeptide/nucleic acid component complex immunogenicity or toxicity, TnpB polypeptide protein or mRNA dose or titer, nucleic acid component dose or titer, TnpB complex dose or titer, TnpB polypeptide protein size, TnpB polypeptide expression level, nucleic acid component expression level, TnpB polypeptide/nucleic acid component molecule complex expression level, TnpB polypeptide spatiotemporal expression, nucleic acid component spatiotemporal expression, TnpB polypeptide/nucleic acid component complex spatiotemporal expression.

It will be understood that the parameters or variables to be optimized as well as the nature of optimization may depend on the (therapeutic) target, the functionality of the composition and/or system, the system mode of delivery, and/or the composition delivery vehicle or expression system.

In an aspect, the invention relates to a method as described herein, comprising optimization of nucleic acid component specificity at the population level. Preferably, said optimization of nucleic acid component specificity comprises minimizing nucleic acid component target site sequence variation across a population and/or minimizing nucleic acid component off-target incidence across a population.

In one embodiment, optimization can result in selection of a TnpB polypeptide that is naturally occurring or is modified. In one embodiment, optimization can result in selection of a TnpB polypeptide that has nuclease, nickase, deaminase, transposase, and/or has one or more effector functionalities deactivated or eliminated. In one embodiment, optimizing a TAMspecificity can include selecting a TnpB polypeptide with a modified TAMspecificity. In one embodiment, optimizing can include selecting a TnpB polypeptide having a minimal size. In one embodiment, optimizing effector protein stability comprises selecting an effector protein having a short half-life while maintaining sufficient activity, such as by selecting an appropriate TnpB polypeptide orthologue having a specific half-life or stability. In one embodiment, optimizing immunogenicity or toxicity comprises minimizing effector protein immunogenicity or toxicity by protein modifications. In one embodiment, optimizing functional specific comprises selecting a protein effector with reduced tolerance of mismatches and/or bulges between the nucleic acid component molecule and one or more target loci.

In one embodiment, optimizing efficacy comprises optimizing overall efficiency, epigenetic tolerance, or both. In one embodiment, maximizing overall efficiency comprises selecting an effector protein with uniform enzyme activity across target loci with varying chromatin complexity, selecting an effector protein with enzyme activity limited to areas of open chromatin accessibility. In one embodiment, chromatin accessibility is measured using one or more of ATAC-seq, or a DNA-proximity ligation assay. In one embodiment, optimizing epigenetic tolerance comprises optimizing methylation tolerance, epigenetic mark competition, or both. In one embodiment, optimizing methylation tolerance comprises selecting an effector protein that modify methylated DNA. In one embodiment, optimizing epigenetic tolerance comprises selecting an effector protein unable to modify silenced regions of a chromosome, selecting an effector protein able to modify silenced regions of a chromosome, or selecting target loci not enriched for epigenetic markers.

In one embodiment, selecting an optimized nucleic acid component molecule comprises optimizing stability, immunogenicity, or both, or other associated parameters or variables as described herein elsewhere.

In one embodiment, optimizing nucleic acid component molecule stability and/or nucleic acid component molecule immunogenicity comprises RNA modification, or other nucleic acid component molecule associated parameters or variables as described herein elsewhere. In one embodiment, the modification comprises removing 1-3 nucleotides form the 3′ end of a target complementarity region of the nucleic acid component molecule. In one embodiment, modification comprises an extended nucleic acid component molecule and/or trans RNA/DNA element that create stable structures in the nucleic acid component molecule that compete with nucleic acid component molecule base pairing at a target of off-target loci, or extended complimentary nucleotides between the nucleic acid component molecule and target sequence, or both.

In one embodiment, the mode of delivery comprises delivering nucleic acid component molecule and/or TnpB polypeptide, delivering nucleic acid component molecule and/or TnpB polypeptide mRNA, or delivery nucleic acid component molecule and/or TnpB polypeptide as a DNA based expression system. In one embodiment, the mode of delivery further comprises selecting a delivery vehicle and/or expression systems from the group consisting of liposomes, lipid particles, nanoparticles, biolistics, or viral-based expression/delivery systems. In one embodiment, expression is spatiotemporal expression is optimized by choice of conditional and/or inducible expression systems, including controllable TnpB polypeptide activity optionally a destabilized TnpB polypeptide and/or a split TnpB polypeptide, and/or cell- or tissue-specific expression system.

The methods as described herein may further involve selection of the mode of delivery. In one embodiment, nucleic acid component and/or TnpB polypeptide are or are to be delivered. In one embodiment, nucleic acid component and/or TnpB polypeptide mRNA are or are to be delivered. In one embodiment, nucleic acid component and/or TnpB polypeptide provided in a DNA-based expression system are, or are to be, delivered. In one embodiment, delivery of the individual system components comprises a combination of the above modes of delivery. In one embodiment, delivery comprises delivering nucleic acid component and/or TnpB polypeptide protein, delivering nucleic acid component and/or TnpB polypeptide mRNA, or delivering nucleic acid component and/or TnpB polypeptide as a DNA based expression system.

The methods as described herein may further involve selection of the composition delivery vehicle and/or expression system. Delivery vehicles and expression systems are described herein elsewhere. By means of example, delivery vehicles of nucleic acids and/or proteins include nanoparticles, liposomes, etc. Delivery vehicles for DNA, such as DNA-based expression systems include for instance biolistics, viral based vector systems (e.g., adenoviral, AAV, lentiviral), etc. the skilled person will understand that selection of the mode of delivery, as well as delivery vehicle or expression system may depend on for instance the cell or tissues to be targeted. In one embodiment, the delivery vehicle and/or expression system for delivering the compositions, systems, or components thereof comprises liposomes, lipid particles, nanoparticles, biolistics, or viral-based expression/delivery systems.

### Considerations for Therapeutic Applications

A consideration in genome editing therapy is the choice of sequence-specific nuclease, such as a variant of a TnpB polypeptide. Each nuclease variant may possess its own unique set of strengths and weaknesses, many of which must be balanced in the context of treatment to maximize therapeutic benefit. For a specific editing therapy to be efficacious, a sufficiently high level of modification must be achieved in target cell populations to reverse disease symptoms. This therapeutic modification ‘threshold’ is determined by the fitness of edited cells following treatment and the amount of gene product necessary to reverse symptoms. With regard to fitness, editing creates three potential outcomes for treated cells relative to their unedited counterparts: increased, neutral, or decreased fitness. In the case of increased fitness, corrected cells may be able and expand relative to their diseased counterparts to mediate therapy. In this case, where edited cells possess a selective advantage, even low numbers of edited cells can be amplified through expansion, providing a therapeutic benefit to the patient. Where the edited cells possess no change in fitness, an increase the therapeutic modification threshold can be warranted. As such, significantly greater levels of editing may be needed to treat diseases, where editing creates a neutral fitness advantage, relative to diseases where editing creates increased fitness for target cells. If editing imposes a fitness disadvantage, as would be the case for restoring function to a tumor suppressor gene in cancer cells, modified cells would be outcompeted by their diseased counterparts, causing the benefit of treatment to be low relative to editing rates. This may be overcome with supplemental therapies to increase the potency and/or fitness of the edited cells relative to the diseased counterparts.

In addition to cell fitness, the amount of gene product necessary to treat disease can also influence the minimal level of therapeutic genome editing that can treat or prevent a disease or a symptom thereof. In cases where a small change in the gene product levels can result in significant changes in clinical outcome, the minimal level of therapeutic genome editing is less relative to cases where a larger change in the gene product levels is needed to gain a clinically relevant response. In one embodiment, the minimal level of therapeutic genome editing can range from 0.1 to 1%, 1-5%, 5-10%, 10-15%, 15-20%, 20-25%, 25-30%, 30-35%, 35-40%, 40-45%. 45-50%, or 50-55%. Thus, where a small change in gene product levels can influence clinical outcomes and diseases where there is a fitness advantage for edited cells, are ideal targets for genome editing therapy, as the therapeutic modification threshold is low enough to permit a high chance of success.

The activity of NHEJ and HDR DSB repair can vary by cell type and cell state. NHEJ is not highly regulated by the cell cycle and is efficient across cell types, allowing for high levels of gene disruption in accessible target cell populations. In contrast, HDR acts primarily during S/G2 phase, and is therefore restricted to cells that are actively dividing, limiting treatments that require precise genome modifications to mitotic cells [Ciccia, A. & Elledge, S. Molecular cell 40, 179-204(2010); Chapman, J. R., et al. Molecular cell 47, 497-510(2012)].

The efficiency of correction via HDR may be controlled by the epigenetic state or sequence of the targeted locus, or the specific repair template configuration (single vs. double stranded, long vs. short homology arms) used [Hacein-Bey-Abina, S., et al. The New England journal of medicine 346, 1185-1193(2002); Gaspar, H. B., et al. Lancet 364, 2181-2187(2004); Beumer, K. J., et al. G3(2013)]. The relative activity of NHEJ and HDR machineries in target cells may also affect gene correction efficiency, as these pathways may compete to resolve DSBs [Beumer, K. J., et al. Proceedings of the National Academy of Sciences of the United States of America 105, 19821-19826(2008)]. HDR also imposes a delivery challenge not seen with NHEJ strategies, as it uses the concurrent delivery of nucleases and repair templates. Thus, these differences can be kept in mind when designing, optimizing, and/or selecting a TnpB polypeptide based therapeutic as described in greater detail elsewhere herein.

TnpB polypeptide-based polynucleotide modification application can include combinations of proteins, small RNA molecules, and/or repair templates, and can make, In one embodiment, delivery of these multiple parts substantially more challenging than, for example, traditional small molecule therapeutics. Two main strategies for delivery of compositions, systems, and components thereof have been developed: ex vivo and in vivo. In one embodiment of ex vivo treatments, diseased cells are removed from a subject, edited and then transplanted back into the patient. In other embodiments, cells from a healthy allogeneic donor are collected, modified using a composition or component thereof, to impart various functionalities and/or reduce immunogenicity, and administered to an allogeneic recipient in need of treatment. Ex vivo editing has the advantage of allowing the target cell population to be well defined and the specific dosage of therapeutic molecules delivered to cells to be specified. The latter consideration may be particularly important when off-target modifications are a concern, as titrating the amount of nuclease may decrease such mutations (Hsu et al., 2013). Another advantage of ex vivo approaches is the typically high editing rates that can be achieved, due to the development of efficient delivery systems for proteins and nucleic acids into cells in culture for research and gene therapy applications.

In vivo polynucleotide modification via compositions, systems, and/or components thereof involves direct delivery of the compositions, systems, and/or components thereof to cell types in their native tissues. In vivo polynucleotide modification via compositions, systems, and/or components thereof allows diseases in which the affected cell population is not amenable to ex vivo manipulation to be treated. Furthermore, delivering compositions, systems, and/or components thereof to cells in situ allows for the treatment of multiple tissue and cell types.

In one embodiment, such as those where viral vector systems are used to generate viral particles to deliver the composition and/or component thereof to a cell, the total cargo size of the composition and/or component thereof should be considered as vector systems can have limits on the size of a polynucleotide that can be expressed therefrom and/or packaged into cargo inside of a viral particle. In one embodiment, the tropism of a vector system, such as a viral vector system, should be considered as it can impact the cell type to which the composition or component thereof can be efficiently and/or effectively delivered.

When delivering a system or component thereof via a viral-based system, it can be important to consider the amount of viral particles that will be needed to achieve a therapeutic effect so as to account for the potential immune response that can be elicited by the viral particles when delivered to a subject or cell(s). When delivering a system or component thereof via a viral based system, it can be important to consider mechanisms of controlling the distribution and/or dosage of the system in vivo. Generally, to reduce the potential for off-target effects, it is optimal but not necessarily required, that the amount of the system be as close to the minimum or least effective dose. In practice this can be challenging to do.

In one embodiment, it can be important to consider the immunogenicity of the system or component thereof. In embodiments where the immunogenicity of the system or component thereof is of concern, the immunogenicity system or component thereof can be reduced. By way of example only, the immunogenicity of the system or component thereof can be reduced using the approach set out in Tangri et al. Accordingly, directed evolution or rational design may be used to reduce the immunogenicity of the TnpB polypeptide in the host species (human or other species).

### Xenotransplantation

The present invention also contemplates use of the composition described herein, e.g., TnpB polypeptide protein systems, to provide RNA-guided DNA nucleases adapted to be used to provide modified tissues for transplantation. For example, RNA-guided DNA nucleases may be used to knockout, knockdown or disrupt selected genes in an animal, such as a transgenic pig (such as the human heme oxygenase-1 transgenic pig line), for example by disrupting expression of genes that encode epitopes recognized by the human immune system, i.e., xenoantigen genes. Candidate porcine genes for disruption may for example include a (1,3)-galactosyltransferase and cytidine monophosphate-N-acetylneuraminic acid hydroxylase genes (see PCT Patent Publication WO 2014/066505). In addition, genes encoding endogenous retroviruses may be disrupted, for example the genes encoding all porcine endogenous retroviruses (see Yang et al., 2015, Genome-wide inactivation of porcine endogenous retroviruses (PERVs), Science 27 Nov. 2015: Vol. 350 no. 6264 pp. 1101-1104). In addition, RNA-guided DNA nucleases may be used to target a site for integration of additional genes in xenotransplant donor animals, such as a human CD55 gene to improve protection against hyperacute rejection.

Embodiments of the invention also relate to methods and compositions related to knocking out genes, amplifying genes and repairing particular mutations associated with DNA repeat instability and neurological disorders (Robert D. Wells, Tetsuo Ashizawa, Genetic Instabilities and Neurological Diseases, Second Edition, Academic Press, Oct. 13, 2011-Medical). Specific aspects of tandem repeat sequences have been found to be responsible for more than twenty human diseases (New insights into repeat instability: role of RNA·DNA hybrids. McIvor E I, Polak U, Napierala M. RNA Biol. 2010 September-October; 7(5): 551-8). The present effector protein systems may be harnessed to correct these defects of genomic instability.

Several further aspects of the invention relate to correcting defects associated with a wide range of genetic diseases which are further described on the website of the National Institutes of Health under at the topic subsection Genetic Disorders (website health.nih.gov/topic/GeneticDisorders). The genetic brain diseases may include but are not limited to Adrenoleukodystrophy, Agenesis of the Corpus Callosum, Aicardi Syndrome, Alpers' Disease, Alzheimer's Disease, Barth Syndrome, Batten Disease, CADASIL, Cerebellar Degeneration, Fabry's Disease, Gerstmann-Straussler-Scheinker Disease, Huntington's Disease and other Triplet Repeat Disorders, Leigh's Disease, Lesch-Nyhan Syndrome, Menkes Disease, Mitochondrial Myopathies and NINDS Colpocephaly. These diseases are further described on the website of the National Institutes of Health under the subsection Genetic Brain Disorders.

### Applications in Plants and Fungi

The compositions, systems, and methods described herein can be used to perform gene or genome interrogation or editing or manipulation in plants and fungi. For example, the applications include investigation and/or selection and/or interrogations and/or comparison and/or manipulations and/or transformation of plant genes or genomes; e.g., to create, identify, develop, optimize, or confer trait(s) or characteristic(s) to plant(s) or to transform a plant or fugus genome. There can accordingly be improved production of plants, new plants with new combinations of traits or characteristics or new plants with enhanced traits. The compositions, systems, and methods can be used with regard to plants in Site-Directed Integration (SDI) or Gene Editing (GE) or any Near Reverse Breeding (NRB) or Reverse Breeding (RB) techniques.

The compositions, systems, and methods herein may be used to confer desired traits (e.g., enhanced nutritional quality, increased resistance to diseases and resistance to biotic and abiotic stress, and increased production of commercially valuable plant products or heterologous compounds) on essentially any plants and fungi, and their cells and tissues. The compositions, systems, and methods may be used to modify endogenous genes or to modify their expression without the permanent introduction into the genome of any foreign gene.

In one embodiment, compositions, systems, and methods may be used in genome editing in plants or where RNAi or similar genome editing techniques have been used previously; see, e.g., Nekrasov, “Plant genome editing made easy: targeted mutagenesis in model and crop plants using the CRISPR-Cas system,” Plant Methods 2013, 9:39 (doi: 10.1186/1746-4811-9-39); Brooks, “Efficient gene editing in tomato in the first generation using the CRISPR-Cas9 system,” Plant Physiology September 2014 pp 114247577; Shan, “Targeted genome modification of crop plants using a CRISPR-Cas system,” Nature Biotechnology 31, 686-688(2013); Feng, “Efficient genome editing in plants using a CRISPR/Cas system,” Cell Research (2013) 23:1229-1232. doi: 10.1038/cr.2013.114; published online 20 Aug. 2013; Xie, “RNA-guided genome editing in plants using a CRISPR-Cas system,” Mol Plant. 2013 November; 6(6): 1975-83. doi: 10.1093/mp/sst119. Epub 2013 Aug. 17; Xu, “Gene targeting using the Agrobacterium tumefaciens-mediated CRISPR-Cas system in rice,” Rice 2014, 7:5(2014), Zhou et al., “Exploiting SNPs for biallelic CRISPR mutations in the outcrossing woody perennial Populus reveals 4-coumarate: CoA ligase specificity and Redundancy,” New Phytologist (2015) (Forum) 1-4 (available online only at www.newphytologist.com); Caliando et al, “Targeted DNA degradation using a CRISPR device stably carried in the host genome, NATURE COMMUNICATIONS 6:6989, DOI: 10.1038/ncomms7989, www.nature.com/naturecommunications DOI: 10.1038/ncomms7989; U.S. Pat. No. 6,603,061-Agrobacterium-Mediated Plant Transformation Method; U.S. Pat. No. 7,868,149-Plant Genome Sequences and Uses Thereof and US 2009/0100536-Transgenic Plants with Enhanced Agronomic Traits, Morrell et al “Crop genomics: advances and applications,” Nat Rev Genet. 2011 Dec. 29; 13(2): 85-96, all the contents and disclosure of each of which are herein incorporated by reference in their entirety. Aspects of utilizing the compositions, systems, and methods may be analogous to the use of the composition in plants, and mention is made of the University of Arizona website “CRISPR-PLANT” (genome.arizona.edu/crispr/) (supported by Penn State and AGI) for directionas to nucleic acid modification in plant systems.

The compositions, systems, and methods may also be used on protoplasts. A “protoplast” refers to a plant cell that has had its protective cell wall completely or partially removed using, for example, mechanical or enzymatic means resulting in an intact biochemical competent unit of living plant that can reform their cell wall, proliferate and regenerate grow into a whole plant under proper growing conditions.

The compositions, systems, and methods may be used for screening genes (e.g., endogenous, mutations) of interest. In some examples, genes of interest include those encoding enzymes involved in the production of a component of added nutritional value or generally genes affecting agronomic traits of interest, across species, phyla, and plant kingdom. By selectively targeting e.g., genes encoding enzymes of metabolic pathways, the genes responsible for certain nutritional aspects of a plant can be identified. Similarly, by selectively targeting genes which may affect a desirable agronomic trait, the relevant genes can be identified. Accordingly, the present invention encompasses screening methods for genes encoding enzymes involved in the production of compounds with a particular nutritional value and/or agronomic traits.

It is also understood that reference herein to animal cells may also apply, mutatis mutandis, to plant or fungal cells unless otherwise apparent; and, the enzymes herein having reduced off-target effects and systems employing such enzymes can be used in plant applications, including those mentioned herein.

In some cases, nucleic acids introduced to plants and fungi may be codon optimized for expression in the plants and fungi. Methods of codon optimization include those described in Kwon K C, et al., Codon Optimization to Enhance Expression Yields Insights into Chloroplast Translation, Plant Physiol. 2016 September; 172(1): 62-77.

The components (e.g., TnpB polypeptide) in the compositions and systems may further comprise one or more functional domains described herein. In some examples, the functional domains may be an exonuclease. Such exonuclease may increase the efficiency of the TnpB polypeptide′ function, e.g., mutagenesis efficiency. An example of the functional domain is Trex2, as described in Weiss T et al., www.biorxiv.org/content/10.1101/2020.04.11.037572v1, doi: doi.org/10.1101/2020.04.11.037572.

### Examples of Plants

The compositions, systems, and methods herein can be used to confer desired traits on essentially any plant. A wide variety of plants and plant cell systems may be engineered for the desired physiological and agronomic characteristics. In general, the term “plant” relates to any various photosynthetic, eukaryotic, unicellular or multicellular organism of the kingdom Plantae characteristically growing by cell division, containing chloroplasts, and having cell walls comprised of cellulose. The term plant encompasses monocotyledonous and dicotyledonous plants.

The compositions, systems, and methods may be used over a broad range of plants, such as for example with dicotyledonous plants belonging to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales; monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g., those belonging to the orders Pinales, Ginkgoales, Cycadales, Araucariales, Cupressales and Gnetales.

The compositions, systems, and methods herein can be used over a broad range of plant species, included in the non-limitative list of dicot, monocot or gymnosperm genera hereunder: Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, and Pseudotsuga.

In one embodiment, target plants and plant cells for engineering include those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Specifically, the plants are intended to comprise without limitation angiosperm and gymnosperm plants such as acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini.

The term plant also encompasses Algae, which are mainly photoautotrophs unified primarily by their lack of roots, leaves and other organs that characterize higher plants. The compositions, systems, and methods can be used over a broad range of “algae” or “algae cells.” Examples of algae include eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). Examples of algae species include those of Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

### Plant Promoters

In order to ensure appropriate expression in a plant cell, the components of the components and systems herein may be placed under control of a plant promoter. A plant promoter is a promoter operable in plant cells. A plant promoter is capable of initiating transcription in plant cells, whether or not its origin is a plant cell. The use of different types of promoters is envisaged.

In some examples, the plant promoter is a constitutive plant promoter, which is a promoter that is able to express the open reading frame (ORF) that it controls in all or nearly all of the plant tissues during all or nearly all developmental stages of the plant (referred to as “constitutive expression”). One example of a constitutive promoter is the cauliflower mosaic virus 35S promoter. In some examples, the plant promoter is a regulated promoter, which directs gene expression not constitutively, but in a temporally- and/or spatially-regulated manner, and includes tissue-specific, tissue-preferred and inducible promoters. Different promoters may direct the expression of a gene in different tissues or cell types, or at different stages of development, or in response to different environmental conditions. In some examples, the plant promoter is a tissue-preferred promoters, which can be utilized to target enhanced expression in certain cell types within a particular plant tissue, for instance vascular cells in leaves or roots or in specific cells of the seed.

Exemplary plant promoters include those obtained from plants, plant viruses, and bacteria such as Agrobacterium or Rhizobium which comprise genes expressed in plant cells. Additional examples of promoters include those described in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

In some examples, a plant promoter may be an inducible promoter, which is inducible and allows for spatiotemporal control of gene editing or gene expression may use a form of energy. The form of energy may include sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. In a particular example, of the components of a light inducible system include a TnpB polypeptide, a light-responsive cytochrome heterodimer (e.g., from Arabidopsis thaliana), and a transcriptional activation/repression domain.

In some examples, the promoter may be a chemical-regulated promotor (where the application of an exogenous chemical induces gene expression) or a chemical-repressible promoter (where application of the chemical represses gene expression). Examples of chemical-inducible promoters include maize ln2-2 promoter (activated by benzene sulfonamide herbicide safeners), the maize GST promoter (activated by hydrophobic electrophilic compounds used as pre-emergent herbicides), the tobacco PR-1 a promoter (activated by salicylic acid), promoters regulated by antibiotics (such as tetracycline-inducible and tetracycline-repressible promoters).

### Stable Integration in the Genome of Plants

In one embodiment, polynucleotides encoding the components of the compositions and systems may be introduced for stable integration into the genome of a plant cell. In some cases, vectors or expression systems may be used for such integration. The design of the vector or the expression system can be adjusted depending on for when, where and under what conditions the nucleic acid component molecule and/or the TnpB polypeptide gene are expressed. In some cases, the polynucleotides may be integrated into an organelle of a plant, such as a plastid, mitochondrion or a chloroplast. The elements of the expression system may be on one or more expression constructs which are either circular such as a plasmid or transformation vector, or non-circular such as linear double stranded DNA.

In one embodiment, the method of integration generally comprises the steps of selecting a suitable host cell or host tissue, introducing the construct(s) into the host cell or host tissue, and regenerating plant cells or plants therefrom. In some examples, the expression system for stable integration into the genome of a plant cell may contain one or more of the following elements: a promoter element that can be used to express the RNA and/or TnpB polypeptide in a plant cell; a 5′ untranslated region to enhance expression; an intron element to further enhance expression in certain cells, such as monocot cells; a multiple-cloning site to provide convenient restriction sites for inserting the nucleic acid component molecule and/or the TnpB polypeptide gene sequences and other desired elements; and a 3′ untranslated region to provide for efficient termination of the expressed transcript.

### Transient Expression in Plants

In one embodiment, the components of the compositions and systems may be transiently expressed in the plant cell. In some examples, the compositions and systems may modify a target nucleic acid only when both the nucleic acid component molecule and the TnpB polypeptide are present in a cell, such that genomic modification can further be controlled. As the expression of the TnpB polypeptide is transient, plants regenerated from such plant cells typically contain no foreign DNA. In certain examples, the TnpB polypeptide is stably expressed and the nucleic acid component molecule sequence is transiently expressed.

DNA and/or RNA (e.g., mRNA) may be introduced to plant cells for transient expression. In such cases, the introduced nucleic acid may be provided in sufficient quantity to modify the cell but do not persist after a contemplated period of time has passed or after one or more cell divisions.

The transient expression may be achieved using suitable vectors. Exemplary vectors that may be used for transient expression include a PEAQ vector (may be tailored for Agrobacterium-mediated transient expression) and Cabbage Leaf Curl virus (CaLCuV), and vectors described in Sainsbury F. et al., Plant Biotechnol J. 2009 September; 7(7): 682-93; and Yin K et al., Scientific Reports volume 5, Article number: 14926(2015).

Combinations of the different methods described above are also envisaged.

Translocation to and/or Expression in Specific Plant Organelles

The compositions and systems herein may comprise elements for translocation to and/or expression in a specific plant organelle.

### Chloroplast Targeting

In one embodiment, it is envisaged that the compositions and systems are used to specifically modify chloroplast genes or to ensure expression in the chloroplast. The compositions and systems (e.g., TnpB polypeptide, nucleic acid components, or their encoding polynucleotides) may be transformed, compartmentalized, and/or targeted to the chloroplast. In an example, the introduction of genetic modifications in the plastid genome can reduce biosafety issues such as gene flow through pollen.

Examples of methods of chloroplast transformation include Particle bombardment, PEG treatment, and microinjection, and the translocation of transformation cassettes from the nuclear genome to the plastid. In some examples, targeting of chloroplasts may be achieved by incorporating in chloroplast localization sequence, and/or the expression construct a sequence encoding a chloroplast transit peptide (CTP) or plastid transit peptide, operably linked to the 5′ region of the sequence encoding the components of the compositions and systems. Additional examples of transforming, targeting and localization of chloroplasts include those described in WO2010061186, Protein Transport into Chloroplasts, 2010, Annual Review of Plant Biology, Vol. 61:157-180, and US20040142476, which are incorporated by reference herein in their entireties.

### Exemplary Applications in Plants

The compositions, systems, and methods may be used to generate genetic variation(s) in a plant (e.g., crop) of interest. One or more, e.g., a library of, nucleic acid components targeting one or more locations in a genome may be provided and introduced into plant cells together with the TnpB polypeptide. For example, a collection of genome-scale point mutations and gene knock-outs can be generated. In some examples, the compositions, systems, and methods may be used to generate a plant part or plant from the cells so obtained and screening the cells for a trait of interest. The target genes may include both coding and non-coding regions. In some cases, the trait is stress tolerant and the method is a method for the generation of stress-tolerant crop varieties.

In one embodiment, the compositions, systems, and methods are used to modify endogenous genes or to modify their expression. The expression of the components may induce targeted modification of the genome, either by direct activity of the TnpB polypeptide and optionally introduction of recombination template DNA, or by modification of genes targeted. The different strategies described herein above allow TnpB polypeptide-mediated targeted genome editing without requiring the introduction of the components into the plant genome.

In some cases, the modification may be performed without the permanent introduction into the genome of the plant of any foreign gene, including those encoding components of the composition herein, so as to avoid the presence of foreign DNA in the genome of the plant. This can be of interest as the regulatory requirements for non-transgenic plants are less rigorous. Components which are transiently introduced into the plant cell are typically removed upon crossing.

For example, the modification may be performed by transient expression of the components of the compositions and systems. The transient expression may be performed by delivering the components of the compositions and systems with viral vectors, delivery into protoplasts, with the aid of particulate molecules such as nanoparticles or CPPs.

Generation of Plants with Desired Traits

The compositions, systems, and methods herein may be used to introduce desired traits to plants. The approaches include introduction of one or more foreign genes to confer a trait of interest, editing or modulating endogenous genes to confer a trait of interest.

### Agronomic Traits

In one embodiment, crop plants can be improved by influencing specific plant traits. Examples of the traits include improved agronomic traits such as herbicide resistance, disease resistance, abiotic stress tolerance, high yield, and superior quality, pesticide-resistance, disease resistance, insect and nematode resistance, resistance against parasitic weeds, drought tolerance, nutritional value, stress tolerance, self-pollination voidance, forage digestibility biomass, and grain yield.

In one embodiment, genes that confer resistance to pests or diseases may be introduced to plants. In cases there are endogenous genes that confer such resistance in plants, their expression and function may be enhanced (e.g., by introducing extra copies, modifications that enhance expression and/or activity).

Examples of genes that confer resistance include plant disease resistance genes (e.g., Cf-9, Pto, RSP2, SIDMR6-1), genes conferring resistance to a pest (e.g., those described in WO96/30517), Bacillus thuringiensis proteins, lectins, Vitamin-binding proteins (e.g., avidin), enzyme inhibitors (e.g., protease or proteinase inhibitors or amylase inhibitors), insect-specific hormones or pheromones (e.g., ecdysteroid or a juvenile hormone, variant thereof, a mimetic based thereon, or an antagonist or agonist thereof) or genes involved in the production and regulation of such hormone and pheromones, insect-specific peptides or neuropeptide, Insect-specific venom (e.g., produced by a snake, a wasp, etc., or analog thereof), Enzymes responsible for a hyperaccumulation of a monoterpene, a sesquiterpene, a steroid, hydroxamic acid, a phenylpropanoid derivative or another nonprotein molecule with insecticidal activity, Enzymes involved in the modification of biologically active molecule (e.g., a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic), molecules that stimulates signal transduction, Viral-invasive proteins or a complex toxin derived therefrom, Developmental-arrestive proteins produced in nature by a pathogen or a parasite, a developmental-arrestive protein produced in nature by a plant, or any combination thereof.

The compositions, systems, and methods may be used to identify, screen, introduce or remove mutations or sequences lead to genetic variability that give rise to susceptibility to certain pathogens, e.g., host specific pathogens. Such approach may generate plants that are non-host resistance, e.g., the host and pathogen are incompatible or there can be partial resistance against all races of a pathogen, typically controlled by many genes and/or also complete resistance to some races of a pathogen but not to other races.

In one embodiment, the compositions, systems, and methods may be used to modify genes involved in plant diseases. Such genes may be removed, inactivated, or otherwise regulated or modified. Examples of plant diseases include those described in [0045]-[0080] of US20140213619A1, which is incorporated by reference herein in its entirety.

In one embodiment, genes that confer resistance to herbicides may be introduced to plants. Examples of genes that confer resistance to herbicides include genes conferring resistance to herbicides that inhibit the growing point or meristem, such as an imidazolinone or a sulfonylurea, genes conferring glyphosate tolerance (e.g., resistance conferred by, e.g., mutant 5-enolpyruvylshikimate-3-phosphate synthase genes, aroA genes and glyphosate acetyl transferase (GAT) genes, respectively), or resistance to other phosphono compounds such as by glufosinate (phosphinothricin acetyl transferase (PAT) genes from Streptomyces species, including Streptomyces hygroscopicus and Streptomyces viridichromogenes), and to pyridinoxy or phenoxy proprionic acids and cyclohexones by ACCase inhibitor-encoding genes), genes conferring resistance to herbicides that inhibit photosynthesis (such as a triazine (psbA and gs+ genes) or a benzonitrile (nitrilase gene), and glutathione S-transferase), genes encoding enzymes detoxifying the herbicide or a mutant glutamine synthase enzyme that is resistant to inhibition, genes encoding a detoxifying enzyme is an enzyme encoding a phosphinothricin acetyltransferase (such as the bar or pat protein from Streptomyces species), genes encoding hydroxyphenylpyruvatedioxygenases (HPPD) inhibitors, e.g., naturally occurring HPPD resistant enzymes, and genes encoding a mutated or chimeric HPPD enzyme.

In one embodiment, genes involved in Abiotic stress tolerance may be introduced to plants. Examples of genes include those capable of reducing the expression and/or the activity of poly(ADP-ribose) polymerase (PARP) gene, transgenes capable of reducing the expression and/or the activity of the PARG encoding genes, genes coding for a plant-functional enzyme of the nicotineamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphorybosyltransferase, enzymes involved in carbohydrate biosynthesis, enzymes involved in the production of polyfructose (e.g., the inulin and levan-type), the production of alpha-1,6 branched alpha-1,4-glucans, the production of alternan, the production of hyaluronan.

In one embodiment, genes that improve drought resistance may be introduced to plants. Examples of genes Ubiquitin Protein Ligase protein (UPL) protein (UPL3), DR02, DR03, ABC transporter, and DREB1A.

### Nutritionally Improved Plants

In one embodiment, the compositions, systems, and methods may be used to produce nutritionally improved plants. In some examples, such plants may provide functional foods, e.g., a modified food or food ingredient that may provide a health benefit beyond the traditional nutrients it contains. In certain examples, such plants may provide nutraceuticals foods, e.g., substances that may be considered a food or part of a food and provides health benefits, including the prevention and treatment of disease. The nutraceutical foods may be useful in the prevention and/or treatment of diseases in animals and humans, e.g., cancers, diabetes, cardiovascular disease, and hypertension.

An improved plant may naturally produce one or more desired compounds and the modification may enhance the level or activity or quality of the compounds. In some cases, the improved plant may not naturally produce the compound(s), while the modification enables the plant to produce such compound(s). In some cases, the compositions, systems, and methods used to modify the endogenous synthesis of these compounds indirectly, e.g., by modifying one or more transcription factors that controls the metabolism of this compound.

Examples of nutritionally improved plants include plants comprising modified protein quality, content and/or amino acid composition, essential amino acid contents, oils and fatty acids, carbohydrates, vitamins and carotenoids, functional secondary metabolites, and minerals. In some examples, the improved plants may comprise or produce compounds with health benefits. Examples of nutritionally improved plants include those described in Newell-McGloughlin, Plant Physiology, July 2008, Vol. 147, pp. 939-953.

Examples of compounds that can be produced include carotenoids (e.g., α-Carotene or β-Carotene), lutein, lycopene, Zeaxanthin, Dietary fiber (e.g., insoluble fibers, β-Glucan, soluble fibers, fatty acids (e.g., @-3 fatty acids, Conjugated linoleic acid, GLA), Flavonoids (e.g., Hydroxycinnamates, flavonols, catechins and tannins), Glucosinolates, indoles, isothiocyanates (e.g., Sulforaphane), Phenolics (e.g., stilbenes, caffeic acid and ferulic acid, epicatechin), Plant stanols/sterols, Fructans, inulins, fructo-oligosaccharides, Saponins, Soybean proteins, Phytoestrogens (e.g., isoflavones, lignans), Sulfides and thiols such as diallyl sulphide, Allyl methyl trisulfide, dithiolthiones, Tannins, such as proanthocyanidins, or any combination thereof.

The compositions, systems, and methods may also be used to modify protein/starch functionality, shelf life, taste/aesthetics, fiber quality, and allergen, antinutrient, and toxin reduction traits.

Examples of genes and nucleic acids that can be modified to introduce the traits include stearyl-ACP desaturase, DNA associated with the single allele which may be responsible for maize mutants characterized by low levels of phytic acid, Tf RAP2.2 and its interacting partner SINAT2, Tf Dof1, and DOF Tf AtDof1.1 (OBP2).

### Modification of Polyploid Plants

The compositions, systems, and methods may be used to modify polyploid plants. Polyploid plants carry duplicate copies of their genomes (e.g., as many as six, such as in wheat). In some cases, the compositions, systems, and methods may be multiplexed to affect all copies of a gene, or to target dozens of genes at once. For instance, the compositions, systems, and methods may be used to simultaneously ensure a loss of function mutation in different genes responsible for suppressing defenses against a disease. The modification may be simultaneous suppression the expression of the TaMLO-A1, TaMLO-B1 and TaMLO-DI nucleic acid sequence in a wheat plant cell and regenerating a wheat plant therefrom, in order to ensure that the wheat plant is resistant to powdery mildew (e.g., as described in WO2015109752).

### Regulation of Fruit-Ripening

The compositions, systems, and methods may be used to regulate ripening of fruits. Ripening is a normal phase in the maturation process of fruits and vegetables. Only a few days after it starts it may render a fruit or vegetable inedible, which can bring significant losses to both farmers and consumers.

In one embodiment, the compositions, systems, and methods are used to reduce ethylene production. In some examples, the compositions, systems, and methods may be used to suppress the expression and/or activity of ACC synthase, insert a ACC deaminase gene or a functional fragment thereof, insert a SAM hydrolase gene or functional fragment thereof, suppress ACC oxidase gene expression.

Alternatively or additionally, the compositions, systems, and methods may be used to modify ethylene receptors (e.g., suppressing ETR1) and/or Polygalacturonase (PG). Suppression of a gene may be achieved by introducing a mutation, an antisense sequence, and/or a truncated copy of the gene to the genome.

### Increasing Storage Life of Plants

In one embodiment, the compositions, systems, and methods are used to modify genes involved in the production of compounds which affect storage life of the plant or plant part. The modification may be in a gene that prevents the accumulation of reducing sugars in potato tubers. Upon high-temperature processing, these reducing sugars react with free amino acids, resulting in brown, bitter-tasting products and elevated levels of acrylamide, which is a potential carcinogen. In particular embodiments, the methods provided herein are used to reduce or inhibit expression of the vacuolar invertase gene (VInv), which encodes a protein that breaks down sucrose to glucose and fructose.

### Reducing Allergens in Plants

In one embodiment, the compositions, systems, and methods are used to generate plants with a reduced level of allergens, making them safer for consumers. To this end, the compositions, systems, and methods may be used to identify and modify (e.g., suppress) one or more genes responsible for the production of plant allergens. Examples of such genes include Lol p5, as well as those in peanuts, soybeans, lentils, peas, lupin, green beans, mung beans, such as those described in Nicolaou et al., Current Opinion in Allergy and Clinical Immunology 2011; 11(3): 222), which is incorporated by reference herein in its entirety.

### Generation of Male Sterile Plants

The compositions, systems, and methods may be used to generate male sterile plants. Hybrid plants typically have advantageous agronomic traits compared to inbred plants. However, for self-pollinating plants, the generation of hybrids can be challenging. In different plant types (e.g., maize and rice), genes have been identified which are important for plant fertility, more particularly male fertility. Plants that are as such genetically altered can be used in hybrid breeding programs.

The compositions, systems, and methods may be used to modify genes involved male fertility, e.g., inactivating (such as by introducing mutations to) genes required for male fertility. Examples of the genes involved in male fertility include cytochrome P450-like gene (MS26) or the meganuclease gene (MS45), and those described in Wan X et al., Mol Plant. 2019 Mar. 4; 12(3): 321-342; and Kim Y J, et al., Trends Plant Sci. 2018 January; 23(1): 53-65.

### Increasing the Fertility Stage in Plants

In one embodiment, the compositions, systems, and methods may be used to prolong the fertility stage of a plant such as of a rice. For instance, a rice fertility stage gene such as Ehd3 can be targeted in order to generate a mutation in the gene and plantlets can be selected for a prolonged regeneration plant fertility stage.

### Production of Early Yield of Products

In one embodiment, the compositions, systems, and methods may be used to produce early yield of the product. For example, flowering process may be modulated, e.g., by mutating flowering repressor gene such as SP5G. Examples of such approaches include those described in Soyk S, et al., Nat Genet. 2017 January; 49(1): 162-168.

### Oil and Biofuel Production

The compositions, systems, and methods may be used to generate plants for oil and biofuel production. Biofuels include fuels made from plant and plant-derived resources. Biofuels may be extracted from organic matter whose energy has been obtained through a process of carbon fixation or are made through the use or conversion of biomass. This biomass can be used directly for biofuels or can be converted to convenient energy containing substances by thermal conversion, chemical conversion, and biochemical conversion. This biomass conversion can result in fuel in solid, liquid, or gas form. Biofuels include bioethanol and biodiesel. Bioethanol can be produced by the sugar fermentation process of cellulose (starch), which may be derived from maize and sugar cane. Biodiesel can be produced from oil crops such as rapeseed, palm, and soybean. Biofuels can be used for transportation.

### Generation of Plants for Production of Vegetable Oils and Biofuels

The compositions, systems, and methods may be used to generate algae (e.g., diatom) and other plants (e.g., grapes) that express or overexpress high levels of oil or biofuels.

In some cases, the compositions, systems, and methods may be used to modify genes involved in the modification of the quantity of lipids and/or the quality of the lipids. Examples of such genes include those involved in the pathways of fatty acid synthesis, e.g., acetyl-CoA carboxylase, fatty acid synthase, 3-ketoacyl_acyl-carrier protein synthase III, glycerol-3-phospate deshydrogenase (G3PDH), Enoyl-acyl carrier protein reductase (Enoyl-ACP-reductase), glycerol-3-phosphate acyltransferase, lysophosphatidic acyl transferase or diacylglycerol acyltransferase, phospholipid: diacylglycerol acyltransferase, phoshatidate phosphatase, fatty acid thioesterase such as palmitoyi protein thioesterase, or malic enzyme activities.

In further embodiments, it is envisaged to generate diatoms that have increased lipid accumulation. This can be achieved by targeting genes that decrease lipid catabolization. Examples of genes include those involved in the activation of triacylglycerol and free fatty acids, β-oxidation of fatty acids, such as genes of acyl-CoA synthetase, 3-ketoacyl-CoA thiolase, acyl-CoA oxidase activity and phosphoglucomutase.

In some examples, algae may be modified for production of oil and biofuels, including fatty acids (e.g., fatty esters such as acid methyl esters (FAME) and fatty acid ethyl esters (FAEE)). Examples of methods of modifying microalgae include those described in Stovicek et al. Metab. Eng. Comm., 2015; 2:1; U.S. Pat. No. 8,945,839; and International Patent Publication No. WO 2015/086795.

In some examples, one or more genes may be introduced (e.g., overexpressed) to the plants (e.g., algae) to produce oils and biofuels (e.g., fatty acids) from a carbon source (e.g., alcohol). Examples of the genes include genes encoding acyl-CoA synthases, ester synthases, thioesterases (e.g., tesA, 'tesA, tesB, fatB, fatB2, fatB3, fatAl, or fatA), acyl-CoA synthases (e.g., fadD, JadK, BH3103, pfl-4354, EAV15023, fadDI, fadD2, RPC_4074,fadDD35, fadDD22, faa39), ester synthases (e.g., synthase/acyl-CoA: diacylglycerl acyltransferase from Simmondsia chinensis, Acinetobacter sp. ADP, Alcanivorax borkumensis, Pseudomonas aeruginosa, Fundibacter jadensis, Arabidopsis thaliana, or Alkaligenes eutrophus, or variants thereof).

Additionally or alternatively, one or more genes in the plants (e.g., algae) may be inactivated (e.g., expression of the genes is decreased). For examples, one or more mutations may be introduced to the genes. Examples of such genes include genes encoding acyl-CoA dehydrogenases (e.g., fade), outer membrane protein receptors, and transcriptional regulator (e.g., repressor) of fatty acid biosynthesis (e.g., fabR), pyruvate formate lyases (e.g., pflB), lactate dehydrogenases (e.g., IdhA).

### Organic Acid Production

In one embodiment, plants may be modified to produce organic acids such as lactic acid. The plants may produce organic acids using sugars, pentose or hexose sugars. To this end, one or more genes may be introduced (e.g., and overexpressed) in the plants. An example of such genes includes LDH gene.

In some examples, one or more genes may be inactivated (e.g., expression of the genes is decreased). For examples, one or more mutations may be introduced to the genes. The genes may include those encoding proteins involved an endogenous metabolic pathway which produces a metabolite other than the organic acid of interest and/or wherein the endogenous metabolic pathway consumes the organic acid.

Examples of genes that can be modified or introduced include those encoding pyruvate decarboxylases (pdc), fumarate reductases, alcohol dehydrogenases (adh), acetaldehyde dehydrogenases, phosphoenolpyruvate carboxylases (ppc), D-lactate dehydrogenases (d-ldh), L-lactate dehydrogenases (l-ldh), lactate 2-monooxygenases, lactate dehydrogenase, cytochrome-dependent lactate dehydrogenases (e.g., cytochrome B2-dependent L-lactate dehydrogenases).

### Enhancing Plant Properties for Biofuel Production

In one embodiment, the compositions, systems, and methods are used to alter the properties of the cell wall of plants to facilitate access by key hydrolyzing agents for a more efficient release of sugars for fermentation. By reducing the proportion of lignin in a plant the proportion of cellulose can be increased. In particular embodiments, lignin biosynthesis may be downregulated in the plant so as to increase fermentable carbohydrates.

In some examples, one or more lignin biosynthesis genes may be down regulated. Examples of such genes include 4-coumarate 3-hydroxylases (C3H), phenylalanine ammonia-lyases (PAL), cinnamate 4-hydroxylases (C4H), hydroxycinnamoyl transferases (HCT), caffeic acid O-methyltransferases (COMT), caffeoyl CoA 3-O-methyltransferases (CCoAOMT), ferulate 5-hydroxylases (F5H), cinnamyl alcohol dehydrogenases (CAD), cinnamoyl CoA-reductases (CCR), 4-coumarate-CoA ligases (4CL), monolignol-lignin-specific glycosyltransferases, and aldehyde dehydrogenases (ALDH), and those described in WO 2008064289.

In some examples, plant mass that produces lower level of acetic acid during fermentation may be reduced. To this end, genes involved in polysaccharide acetylation (e.g., Cas1L and those described in WO 2010096488) may be inactivated.

### Other Microorganisms for Oils and Biofuel Production

In one embodiment, microorganisms other than plants may be used for production of oils and biofuels using the compositions, systems, and methods herein. Examples of the microorganisms include those of the genus of Escherichia, Bacillus, Lactobacillus, Rhodococcus, Synechococcus, Synechoystis, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces.

### Plant Cultures and Regeneration

In one embodiment, the modified plants or plant cells may be cultured to regenerate a whole plant which possesses the transformed or modified genotype and thus the desired phenotype. Examples of regeneration techniques include those relying on manipulation of certain phytohormones in a tissue culture growth medium, relying on a biocide and/or herbicide marker which has been introduced together with the desired nucleotide sequences, obtaining from cultured protoplasts, plant callus, explants, organs, pollens, embryos or parts thereof.

### Detecting Modifications in the Plant Genome-Selectable Markers

When the compositions, systems, and methods are used to modify a plant, suitable methods may be used to confirm and detect the modification made in the plant. In some examples, when a variety of modifications are made, one or more desired modifications or traits resulting from the modifications may be selected and detected. The detection and confirmation may be performed by biochemical and molecular biology techniques such as Southern analysis, PCR, Northern blot, S1 RNase protection, primer-extension or reverse transcriptase-PCR, enzymatic assays, ribozyme activity, gel electrophoresis, Western blot, immunoprecipitation, enzyme-linked immunoassays, in situ hybridization, enzyme staining, and immunostaining.

In some cases, one or more markers, such as selectable and detectable markers, may be introduced to the plants. Such markers may be used for selecting, monitoring, isolating cells and plants with desired modifications and traits. A selectable marker can confer positive or negative selection and is conditional or non-conditional on the presence of external substrates. Examples of such markers include genes and proteins that confer resistance to antibiotics, such as hygromycin (hpt) and kanamycin (nptII), and genes that confer resistance to herbicides, such as phosphinothricin (bar) and chlorosulfuron (als), enzyme capable of producing or processing a colored substances (e.g., the β-glucuronidase, luciferase, B or C1 genes).

### Applications in Fungi

The compositions, systems, and methods described herein can be used to perform efficient and cost effective gene or genome interrogation or editing or manipulation in fungi or fungal cells, such as yeast. The approaches and applications in plants may be applied to fungi as well.

A fungal cell may be any type of eukaryotic cell within the kingdom of fungi, such as phyla of Ascomycota, Basidiomycota, Blastocladiomycota, Chytridiomycota, Glomeromycota, Microsporidia, and Neocallimastigomycota. Examples of fungi or fungal cells in include yeasts, molds, and filamentous fungi.

In one embodiment, the fungal cell is a yeast cell. A yeast cell refers to any fungal cell within the phyla Ascomycota and Basidiomycota. Examples of yeasts include budding yeast, fission yeast, and mold, S. cerervisiae, Kluyveromyces marxianus, Issatchenkia orientalis, Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, Pichia kudriavzevii and Candida acidothermophilum).

In one embodiment, the fungal cell is a filamentous fungal cell, which grow in filaments, e.g., hyphae or mycelia. Examples of filamentous fungal cells include Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In one embodiment, the fungal cell is of an industrial strain. Industrial strains include any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes include fermentation (e.g., in production of food or beverage products), distillation, biofuel production, production of a compound, and production of a polypeptide. Examples of industrial strains include, without limitation, JAY270 and ATCC4124.

In one embodiment, the fungal cell is a polyploid cell whose genome is present in more than one copy. Polyploid cells include cells naturally found in a polyploid state, and cells that has been induced to exist in a polyploid state (e.g., through specific regulation, alteration, inactivation, activation, or modification of meiosis, cytokinesis, or DNA replication). A polyploid cell may be a cell whose entire genome is polyploid, or a cell that is polyploid in a particular genomic locus of interest. In some examples, the abundance of nucleic acid component molecule may more often be a rate-limiting component in genome engineering of polyploid cells than in haploid cells, and thus the methods using the composition described herein may take advantage of using certain fungal cell types.

In one embodiment, the fungal cell is a diploid cell, whose genome is present in two copies. Diploid cells include cells naturally found in a diploid state, and cells that have been induced to exist in a diploid state (e.g., through specific regulation, alteration, inactivation, activation, or modification of meiosis, cytokinesis, or DNA replication). A diploid cell may refer to a cell whose entire genome is diploid, or it may refer to a cell that is diploid in a particular genomic locus of interest.

In one embodiment, the fungal cell is a haploid cell, whose genome is present in one copy. Haploid cells include cells naturally found in a haploid state, or cells that have been induced to exist in a haploid state (e.g., through specific regulation, alteration, inactivation, activation, or modification of meiosis, cytokinesis, or DNA replication). A haploid cell may refer to a cell whose entire genome is haploid, or it may refer to a cell that is haploid in a particular genomic locus of interest.

The compositions and systems, and nucleic acid encoding thereof may be introduced to fungi cells using the delivery systems and methods herein. Examples of delivery systems include lithium acetate treatment, bombardment, electroporation, and those described in Kawai et al., 2010, Bioeng Bugs. 2010 November-December; 1(6): 395-403.

In some examples, a yeast expression vector (e.g., those with one or more regulatory elements) may be used. Examples of such vectors include a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2u plasmids, yeast integrative plasmids, yeast replicative plasmids, shuttle vectors, and episomal plasmids.

### Biofuel and Materials Production by Fungi

In one embodiment, the compositions, systems, and methods may be used for generating modified fungi for biofuel and material productions. For instance, the modified fungi for production of biofuel or biopolymers from fermentable sugars and optionally to be able to degrade plant-derived lignocellulose derived from agricultural waste as a source of fermentable sugars. Foreign genes required for biofuel production and synthesis may be introduced into fungi. In some examples, the genes may encode enzymes involved in the conversion of pyruvate to ethanol or another product of interest, degrade cellulose (e.g., cellulase), endogenous metabolic pathways which compete with the biofuel production pathway.

In some examples, the compositions, systems, and methods may be used for generating and/or selecting yeast strains with improved xylose or cellobiose utilization, isoprenoid biosynthesis, and/or lactic acid production. One or more genes involved in the metabolism and synthesis of these compounds may be modified and/or introduced to yeast cells. Examples of the methods and genes include lactate dehydrogenase, PDC1 and PDC5, and those described in Ha, S., et al. (2011) Proc. Natl. Acad. Sci. USA 108(2): 504-9 and Galazka, J. M., et al. (2010) Science 330(6000): 84-6; Jakočiũnas T et al., Metab Eng. 2015 March; 28:213-222; Stovicek V, et al., FEMS Yeast Res. 2017 Aug. 1; 17(5).

### Improved Plants and Yeast Cells

The present disclosure further provides improved plants and fungi. The improved and fungi may comprise one or more genes introduced, and/or one or more genes modified by the compositions, systems, and methods herein. The improved plants and fungi may have increased food or feed production (e.g., higher protein, carbohydrate, nutrient or vitamin levels), oil and biofuel production (e.g., methanol, ethanol), tolerance to pests, herbicides, drought, low or high temperatures, excessive water, etc.

The plants or fungi may have one or more parts that are improved, e.g., leaves, stems, roots, tubers, seeds, endosperm, ovule, and pollen. The parts may be viable, nonviable, regeneratable, and/or non-regeneratable.

The improved plants and fungi may include gametes, seeds, embryos, either zygotic or somatic, progeny and/or hybrids of improved plants and fungi. The progeny may be a clone of the produced plant or fungi, or may result from sexual reproduction by crossing with other individuals of the same species to introgress further desirable traits into their offspring. The cell may be in vivo or ex vivo in the cases of multicellular organisms, particularly plants.

### Further Applications in Plants

Further applications of the compositions, systems, and methods on plants and fungi include visualization of genetic element dynamics (e.g., as described in Chen B, et al., Cell. 2013 Dec. 19; 155(7): 1479-91), targeted gene disruption positive-selection in vitro and in vivo (as described in Malina A et al., Genes Dev. 2013 Dec. 1; 27(23): 2602-14), epigenetic modification such as using fusion of TnpB polypeptide and histone-modifying enzymes (e.g., as described in Rusk N, Nat Methods. 2014 January; 11(1): 28), identifying transcription regulators (e.g., as described in Waldrip Z J, Epigenetics. 2014 September; 9(9): 1207-11), anti-virus treatment for both RNA and DNA viruses (e.g., as described in Price A A, et al., Proc Natl Acad Sci USA. 2015 May 12; 112(19): 6164-9; Ramanan V et al., Sci Rep. 2015 Jun. 2; 5:10833), alteration of genome complexity such as chromosome numbers (e.g., as described in Karimi-Ashtiyani R et al., Proc Natl Acad Sci USA. 2015 Sep. 8; 112(36): 11211-6; Anton T, et al., Nucleus. 2014 March-April; 5(2): 163-72), self-cleavage of the composition for controlled inactivation/activation (e.g., as described Sugano S S et al., Plant Cell Physiol. 2014 March; 55(3): 475-81), multiplexed gene editing (as described in Kabadi A M et al., Nucleic Acids Res. 2014 Oct. 29; 42(19): e147), development of kits for multiplex genome editing (as described in Xing H L et al., BMC Plant Biol. 2014 Nov. 29; 14:327), starch production (as described in Hebelstrup K H et al., Front Plant Sci. 2015 Apr. 23; 6:247), targeting multiple genes in a family or pathway (e.g., as described in Ma X et al., Mol Plant. 2015 August; 8(8): 1274-84), regulation of non-coding genes and sequences (e.g., as described in Lowder L G, et al., Plant Physiol. 2015 October; 169(2): 971-85), editing genes in trees (e.g., as described in Belhaj K et al., Plant Methods. 2013 Oct. 11; 9(1): 39; Harrison M M, et al., Genes Dev. 2014 Sep. 1; 28(17): 1859-72; Zhou X et al., New Phytol. 2015 October; 208(2): 298-301), introduction of mutations for resistance to host-specific pathogens and pests.

Additional examples of modifications of plants and fungi that may be performed using the compositions, systems, and methods include analogous modifications described in International Patent Publication Nos. WO2016/099887, WO2016/025131, WO2016/073433, WO2017/066175, WO2017/100158, WO 2017/105991, WO2017/106414, WO2016/100272, WO2016/100571, WO 2016/100568, WO 2016/100562, and WO 2017/019867.

### Applications in Non-Human Animals

The compositions, systems, and methods may be used to study and modify non-human animals, e.g., introducing desirable traits and disease resilience, treating diseases, facilitating breeding, etc. In one embodiment, the compositions, systems, and methods may be used to improve breeding and introducing desired traits, e.g., increasing the frequency of trait-associated alleles, introgression of alleles from other breeds/species without linkage drag, and creation of de novo favorable alleles. Genes and other genetic elements that can be targeted may be screened and identified. Examples of application and approaches include those described in Tait-Burkard C, et al., Livestock 2.0—genome editing for fitter, healthier, and more productive farmed animals. Genome Biol. 2018 Nov. 26; 19(1): 204; Lillico S, Agricultural applications of genome editing in farmed animals. Transgenic Res. 2019 August; 28 (Suppl 2): 57-60; Houston R D, et al., Harnessing genomics to fast-track genetic improvement in aquaculture. Nat Rev Genet. 2020 Apr. 16. doi: 10.1038/s41576-020-0227-y, which are incorporated herein by reference in their entireties. Applications described in other sections such as therapeutic, diagnostic, etc. can also be used on the animals herein.

The compositions, systems, and methods may be used on animals such as fish, amphibians, reptiles, mammals, and birds. The animals may be farm and agriculture animals, or pets. Examples of farm and agriculture animals include horses, goats, sheep, swine, cattle, llamas, alpacas, and birds, e.g., chickens, turkeys, ducks, and geese. The animals may be a non-human primate, e.g., baboons, capuchin monkeys, chimpanzees, lemurs, macaques, marmosets, tamarins, spider monkeys, squirrel monkeys, and vervet monkeys. Examples of pets include dogs, cats, horses, wolfs, rabbits, ferrets, gerbils, hamsters, chinchillas, fancy rats, guinea pigs, canaries, parakeets, and parrots.

In one embodiment, one or more genes may be introduced (e.g., overexpressed) in the animals to obtain or enhance one or more desired traits. Growth hormones, insulin-like growth factors (IGF-1) may be introduced to increase the growth of the animals, e.g., pigs or salmon (such as described in Pursel V G et al., J Reprod Fertil Suppl. 1990; 40:235-45; Waltz E, Nature. 2017; 548:148). Fat-1 gene (e.g., from C elegans) may be introduced for production of larger ratio of n-3 to n-6 fatty acids may be induced, e.g., in pigs (such as described in Li M, et al., Genetics. 2018; 8:1747-54). Phytase (e.g., from E coli) xylanase (e.g., from Aspergillus niger), beta-glucanase (e.g., from Bacillus lichenformis) may be introduced to reduce the environmental impact through phosphorous and nitrogen release reduction, e.g., in pigs (such as described in Golovan S P, et al., Nat Biotechnol. 2001; 19:741-5; Zhang X et al., elife. 2018). snucleic acid component decoy may be introduced to induce avian influenza resilience e.g., in chicken (such as described in Lyall et al., Science. 2011; 331:223-6). Lysozyme or lysostaphin may be introduced to induce mastitis resilience e.g., in goat and cow (such as described in Maga E A et al., Foodborne Pathog Dis. 2006; 3:384-92; Wall R J, et al., Nat Biotechnol. 2005; 23:445-51). Histone deacetylase such as HDAC6 may be introduced to induce PRRSV resilience, e.g., in pig (such as described in Lu T., et al., PLOS One. 2017; 12: e0169317). CD163 may be modified (e.g., inactivated or removed) to introduce PRRSV resilience in pigs (such as described in Prather R S et al . . . , Sci Rep. 2017 Oct. 17; 7(1): 13371). Similar approaches may be used to inhibit or remove viruses and bacteria (e.g., Swine Influenza Virus (SIV) strains which include influenza C and the subtypes of influenza A known as H1N1, HIN2, H2N1, H3N1, H3N2, and H2N3, as well as pneumonia, meningitis and oedema) that may be transmitted from animals to humans.

In one embodiment, one or more genes may be modified or edited for disease resistance and production traits. Myostatin (e.g., GDF8) may be modified to increase muscle growth, e.g., in cow, sheep, goat, catfish, and pig (such as described in Crispo M et al., PLOS One. 2015; 10: e0136690; Wang X, et al., Anim Genet. 2018; 49:43-51; Khalil K, et al., Sci Rep. 2017; 7:7301; Kang J-D, et al., RSC Adv. 2017; 7:12541-9). Pc POLLED may be modified to induce horlessness, e.g., in cow (such as described in Carlson D F et al., Nat Biotechnol. 2016; 34:479-81). KISSIR may be modified to induce boretaint (hormone release during sexual maturity leading to undesired meat taste), e.g., in pigs. Dead end protein (dnd) may be modified to induce sterility, e.g., in salmon (such as described in Wargelius A, et al., Sci Rep. 2016; 6:21284). Nano2 and DDX may be modified to induce sterility (e.g., in surrogate hosts), e.g., in pigs and chicken (such as described Park K-E, et al., Sci Rep. 2017; 7:40176; Taylor L et al., Development. 2017; 144:928-34). CD163 may be modified to induce PRRSV resistance, e.g., in pigs (such as described in Whitworth K M, et al., Nat Biotechnol. 2015; 34:20-2). RELA may be modified to induce ASFV resilience, e.g., in pigs (such as described in Lillico S G, et al., Sci Rep. 2016; 6:21645). CD18 may be modified to induce Mannheimia (Pasteurella) haemolytica resilience, e.g., in cows (such as described in Shanthalingam S, et al., roc Natl Acad Sci USA. 2016; 113:13186-90). NRAMP1 may be modified to induce tuberculosis resilience, e.g., in cows (such as described in Gao Y et al., Genome Biol. 2017; 18:13). Endogenous retrovirus genes may be modified or removed for xenotransplantation such as described in Yang L, et al. Science. 2015; 350:1101-4; Niu D et al., Science. 2017; 357:1303-7). Negative regulators of muscle mass (e.g., Myostatin) may be modified (e.g., inactivated) to increase muscle mass, e.g., in dogs (as described in Zou Q et al., J Mol Cell Biol. 2015 December; 7(6): 580-3).

Animals such as pigs with severe combined immunodeficiency (SCID) may generated (e.g., by modifying RAG2) to provide useful models for regenerative medicine, xenotransplantation (discussed also elsewhere herein), and tumor development. Examples of methods and approaches include those described Lee K, et al., Proc Natl Acad Sci USA. 2014 May 20; 111(20): 7260-5; and Schomberg et al. FASEB Journal, April 2016; 30(1): Suppl 571.1.

SNPs in the animals may be modified. Examples of methods and approaches include those described Tan W. et al., Proc Natl Acad Sci USA. 2013 Oct. 8; 110(41): 16526-31; Mali P, et al., Science. 2013 Feb. 15; 339(6121): 823-6.

Stem cells (e.g., induced pluripotent stem cells) may be modified and differentiated into desired progeny cells, e.g., as described in Heo Y T et al., Stem Cells Dev. 2015 Feb. 1; 24(3): 393-402.

Profile analysis (such as Igenity) may be performed on animals to screen and identify genetic variations related to economic traits. The genetic variations may be modified to introduce or improve the traits, such as carcass composition, carcass quality, maternal and reproductive traits and average daily gain.

### Detection Compositions and Methods of Detection

In another aspect, embodiments disclosed herein are directed to polynucleotide detection compositions, systems and methods. The detection composition may comprise any of the TnpB polypeptides and any one or more ωRNAs discussed above. In the addition, the compositions and system may comprise a detection construct. In one example embodiment, the detection construct comprises at least a portion of single-stranded polynucleotide. The one or more ωRNAs are configured to bind a target sequence on a target polypeptide. Binding of the TnpB complex to the target sequence activates TnpB cleavage activity and may further activate TnpB collateral activity whereby TnpB subsequently cleaves non-target single-stranded polynucleotides in an ωRNA-independent fashion. Accordingly, the detection construct can be configured so that a detectable signal is generated upon cleavage of the single-stranded portion of the detection constructs thereby indicating the present of the target sequence in a sample. Example detection constructs are discussed in further detail below. In further example embodiments, the compositions may further comprise amplification reagents. Amplification reagents may comprise primers and polymerase and/or reverse transcriptases needed to amplify the target sequence. In on example embodiment, the amplification reagents are isothermal amplification reagents. In other example embodiments, the compositions and systems may further comprise quick extraction solutions that allow for detection of target sequences in crude samples or with minimal purification prior to amplification and/or detection.

### Detection Construct

The systems and methods described herein comprise a detection construct. As used herein, a “detection construct” refers to a molecule that can be cleaved or otherwise deactivated by an activated TnpB system protein described herein. The term “detection construct” may also be referred to in the alternative as a “masking construct.” Depending on the nuclease activity of the TnpB protein and the methods utilized, the masking construct may be a RNA-based masking construct or a DNA-based masking construct. The Nucleic Acid-based masking constructs comprises a nucleic acid element that is cleavable by a TnpB protein. Cleavage of the nucleic acid element releases agents or produces conformational changes that allow a detectable signal to be produced. Example constructs demonstrating how the nucleic acid element may be used to prevent or mask generation of detectable signal are described below and embodiments of the invention comprise variants of the same. Prior to cleavage, or when the masking construct is in an ‘active’ state, the masking construct blocks the generation or detection of a positive detectable signal. In one embodiment, detection constructs are designed for cutting motifs of particular TnpB proteins.

It will be understood that in certain example embodiments a minimal background signal may be produced in the presence of an active masking construct. A positive detectable signal may be any signal that can be detected using optical, fluorescent, chemiluminescent, electrochemical or other detection methods known in the art. The term “positive detectable signal” is used to differentiate from other detectable signals that may be detectable in the presence of the masking construct. For example, in one embodiment a first signal may be detected when the masking agent is present or when a TnpB system has not been activated (i.e., a negative detectable signal), which then converts to a second signal (e.g., the positive detectable signal) upon detection of the target molecules and cleavage or deactivation of the masking agent, or upon activation of the TnpB protein. The positive detectable signal, then, is a signal detected upon activation of the TnpB protein, and may be, in a colorimetric or fluorescent assay, a decrease in fluorescence or color relative to a control or an increase in fluorescence or color relative to a control, depending on the configuration of the lateral flow substrate, and as described further herein.

In certain example embodiments, the masking construct may comprise an HCR initiator sequence and a cutting motif, or a cleavable structural element, such as a loop or hairpin, that prevents the initiator from initiating the HCR reaction. The cutting motif may be preferentially cut by one of the activated TnpB effector proteins. Upon cleavage of the cutting motif or structure element by an activated TnpB protein, the initiator is then released to trigger the HCR reaction, detection thereof indicating the presence of one or more targets in the sample. In certain example embodiments, the masking construct comprises a hairpin with an RNA loop. When an activated TnpB protein cuts the RNA loop, the initiator can be released to trigger the HCR reaction.

In certain example embodiments, the masking construct may suppress generation of a gene product. The gene product may be encoded by a reporter construct that is added to the sample. The masking construct may be an interfering RNA involved in an RNA interference pathway, such as a short hairpin RNA (shRNA) or small interfering RNA (siRNA). The masking construct may also comprise microRNA (miRNA). While present, the masking construct suppresses expression of the gene product. The gene product may be a fluorescent protein or other RNA transcript or proteins that would otherwise be detectable by a labeled probe, aptamer, or antibody but for the presence of the masking construct. Upon activation of the effector protein the masking construct is cleaved or otherwise silenced allowing for expression and detection of the gene product as the positive detectable signal. In preferred embodiments, the masking constructs comprise two or more detectable signals, for example, fluorescent signals, that can be read on different channels of a fluorimeter.

In specific embodiments, the masking construct comprises a silencing RNA that suppresses generation of a gene product encoded by a reporting construct, wherein the gene product generates the detectable positive signal when expressed.

In certain example embodiments, the masking construct may sequester one or more reagents needed to generate a detectable positive signal such that release of the one or more reagents from the masking construct results in generation of the detectable positive signal. The one or more reagents may combine to produce a colorimetric signal, a chemiluminescent signal, a fluorescent signal, or any other detectable signal and may comprise any reagents known to be suitable for such purposes. In certain example embodiments, the one or more reagents are sequestered by RNA aptamers that bind the one or more reagents. The one or more reagents are released when the effector protein is activated upon detection of a target molecule and the RNA or DNA aptamers are degraded.

In certain example embodiments, the masking construct may be immobilized on a solid substrate in an individual discrete volume (defined further below) and sequesters a single reagent. For example, the reagent may be a bead comprising a dye. When sequestered by the immobilized reagent, the individual beads are too diffuse to generate a detectable signal, but upon release from the masking construct are able to generate a detectable signal, for example by aggregation or simple increase in solution concentration. In certain example embodiments, the immobilized masking agent is an RNA- or DNA-based aptamer that can be cleaved by the activated effector protein upon detection of a target molecule.

In certain other example embodiments, the masking construct binds to an immobilized reagent in solution thereby blocking the ability of the reagent to bind to a separate labeled binding partner that is free in solution. Thus, upon application of a washing step to a sample, the labeled binding partner can be washed out of the sample in the absence of a target molecule. However, if the effector protein is activated, the masking construct is cleaved to a degree sufficient to interfere with the ability of the masking construct to bind the reagent thereby allowing the labeled binding partner to bind to the immobilized reagent. Thus, the labeled binding partner remains after the wash step indicating the presence of the target molecule in the sample. In certain aspects, the masking construct that binds the immobilized reagent is a DNA or RNA aptamer. The immobilized reagent may be a protein and the labeled binding partner may be a labeled antibody. Alternatively, the immobilized reagent may be streptavidin and the labeled binding partner may be labeled biotin. The label on the binding partner used in the above embodiments may be any detectable label known in the art. In addition, other known binding partners may be used in accordance with the overall design described herein.

In certain example embodiments, the masking construct may comprise a ribozyme. Ribozymes are RNA molecules having catalytic properties. Ribozymes, both naturally and engineered, comprise or consist of RNA that may be targeted by the effector proteins disclosed herein. The ribozyme may be selected or engineered to catalyze a reaction that either generates a negative detectable signal or prevents generation of a positive control signal. Upon deactivation of the ribozyme by the activated effector protein the reaction generating a negative control signal, or preventing generation of a positive detectable signal, is removed thereby allowing a positive detectable signal to be generated. In one example embodiment, the ribozyme may catalyze a colorimetric reaction causing a solution to appear as a first color. When the ribozyme is deactivated, the solution then turns to a second color, the second color being the detectable positive signal. An example of how ribozymes can be used to catalyze a colorimetric reaction are described in Zhao et al. “Signal amplification of glucosamine-6-phosphate based on ribozyme glmS,” Biosens Bioelectron. 2014; 16:337-42, and provide an example of how such a system could be modified to work in the context of the embodiments disclosed herein. Alternatively, ribozymes, when present can generate cleavage products of, for example, RNA transcripts. Thus, detection of a positive detectable signal may comprise detection of non-cleaved RNA transcripts that are only generated in the absence of the ribozyme.

In one embodiment, the masking construct may be a ribozyme that generates a negative detectable signal, and wherein a positive detectable signal is generated when the ribozyme is deactivated.

In certain example embodiments, the one or more reagents is a protein, such as an enzyme, capable of facilitating generation of a detectable signal, such as a colorimetric, chemiluminescent, or fluorescent signal, that is inhibited or sequestered such that the protein cannot generate the detectable signal by the binding of one or more DNA or RNA aptamers to the protein. Upon activation of the effector proteins disclosed herein, the DNA or RNA aptamers are cleaved or degraded to an extent that they no longer inhibit the protein's ability to generate the detectable signal. In certain example embodiments, the aptamer is a thrombin inhibitor aptamer. In certain example embodiments the thrombin inhibitor aptamer has a sequence of GGGAACAAAGCUGAAGUACUUACCC (SEQ ID NO: 236). When this aptamer is cleaved, thrombin will become active and will cleave a peptide colorimetric or fluorescent substrate. In certain example embodiments, the colorimetric substrate is para-nitroanilide (pNA) covalently linked to the peptide substrate for thrombin. Upon cleavage by thrombin, pNA is released and becomes yellow in color and easily visible to the eye. In certain example embodiments, the fluorescent substrate is 7-amino-4-methylcoumarin a blue fluorophore that can be detected using a fluorescence detector. Inhibitory aptamers may also be used for horseradish peroxidase (HRP), beta-galactosidase, or calf alkaline phosphatase (CAP) and within the general principals laid out above.

In one embodiment, RNAse or DNAse activity is detected colorimetrically via cleavage of enzyme-inhibiting aptamers. One potential mode of converting DNAse or RNAse activity into a colorimetric signal is to couple the cleavage of a DNA or RNA aptamer with the re-activation of an enzyme that is capable of producing a colorimetric output. In the absence of RNA or DNA cleavage, the intact aptamer will bind to the enzyme target and inhibit its activity. The advantage of this readout system is that the enzyme provides an additional amplification step: once liberated from an aptamer via collateral activity (e.g., TnpB collateral activity), the colorimetric enzyme will continue to produce colorimetric product, leading to a multiplication of signal.

In one embodiment, an existing aptamer that inhibits an enzyme with a colorimetric readout is used. Several aptamer/enzyme pairs with colorimetric readouts exist, such as thrombin, protein C, neutrophil elastase, and subtilisin. These proteases have colorimetric substrates based upon pNA and are commercially available. In one embodiment, a novel aptamer targeting a common colorimetric enzyme is used. Common and robust enzymes, such as beta-galactosidase, horseradish peroxidase, or calf intestinal alkaline phosphatase, could be targeted by engineered aptamers designed by selection strategies such as SELEX. Such strategies allow for quick selection of aptamers with nanomolar binding efficiencies and could be used for the development of additional enzyme/aptamer pairs for colorimetric readout.

In one embodiment, the masking construct may be a DNA or RNA aptamer and/or may comprise a DNA or RNA-tethered inhibitor.

In one embodiment, the masking construct may comprise a DNA or RNA oligonucleotide to which a detectable ligand and a masking component are attached.

In one embodiment, RNAse or DNase activity is detected colorimetrically via cleavage of RNA-tethered inhibitors. Many common colorimetric enzymes have competitive, reversible inhibitors: for example, beta-galactosidase can be inhibited by galactose. Many of these inhibitors are weak, but their effect can be increased by increases in local concentration. By linking local concentration of inhibitors to DNase RNAse activity, colorimetric enzyme and inhibitor pairs can be engineered into DNase and RNAse sensors. The colorimetric DNase or RNAse sensor based upon small-molecule inhibitors involves three components: the colorimetric enzyme, the inhibitor, and a bridging RNA or DNA that is covalently linked to both the inhibitor and enzyme, tethering the inhibitor to the enzyme. In the uncleaved configuration, the enzyme is inhibited by the increased local concentration of the small molecule; when the DNA or RNA is cleaved (e.g., by TnpB collateral cleavage), the inhibitor will be released and the colorimetric enzyme will be activated.

In one embodiment, the aptamer or DNA- or RNA-tethered inhibitor may sequester an enzyme, wherein the enzyme generates a detectable signal upon release from the aptamer or DNA or RNA tethered inhibitor by acting upon a substrate. In one embodiment, the aptamer may be an inhibitor aptamer that inhibits an enzyme and prevents the enzyme from catalyzing generation of a detectable signal from a substance. In one embodiment, the DNA- or RNA-tethered inhibitor may inhibit an enzyme and may prevent the enzyme from catalyzing generation of a detectable signal from a substrate.

In one embodiment, RNAse activity is detected colorimetrically via formation and/or activation of G-quadruplexes. G quadruplexes in DNA can complex with heme (iron (III)-protoporphyrin IX) to form a DNAzyme with peroxidase activity. When supplied with a peroxidase substrate (e.g., ABTS: (2,2′-Azinobis [3-ethylbenzothiazoline-6-sulfonic acid]-diammonium salt)), the G-quadruplex-heme complex in the presence of hydrogen peroxide causes oxidation of the substrate, which then forms a green color in solution. An example G-quadruplex forming DNA sequence is: GGGTAGGGCGGGTTGGGA (SEQ ID NO: 237). By hybridizing an additional DNA or RNA sequence, referred to herein as a “staple,” to this DNA aptamer, formation of the G-quadraplex structure will be limited. Upon collateral activation, the staple will be cleaved allowing the G quadraplex to form and heme to bind. This strategy is particularly appealing because color formation is enzymatic, meaning there is additional amplification beyond collateral activation.

In one embodiment, the masking construct may comprise an RNA oligonucleotide designed to bind a G-quadruplex forming sequence, wherein a G-quadruplex structure is formed by the G-quadruplex forming sequence upon cleavage of the masking construct, and wherein the G-quadruplex structure generates a detectable positive signal.

In certain example embodiments, the masking construct may be immobilized on a solid substrate in an individual discrete volume (defined further below) and sequesters a single reagent. For example, the reagent may be a bead comprising a dye. When sequestered by the immobilized reagent, the individual beads are too diffuse to generate a detectable signal, but upon release from the masking construct are able to generate a detectable signal, for example by aggregation or simple increase in solution concentration. In certain example embodiments, the immobilized masking agent is a DNA- or RNA-based aptamer that can be cleaved by the activated effector protein upon detection of a target molecule.

In one example embodiment, the masking construct comprises a detection agent that changes color depending on whether the detection agent is aggregated or dispersed in solution. For example, certain nanoparticles, such as colloidal gold, undergo a visible purple to red color shift as they move from aggregates to dispersed particles. Accordingly, in certain example embodiments, such detection agents may be held in aggregate by one or more bridge molecules. At least a portion of the bridge molecule comprises RNA or DNA. Upon activation of the effector proteins disclosed herein, the RNA or DNA portion of the bridge molecule is cleaved allowing the detection agent to disperse and resulting in the corresponding change in color. In certain example embodiments, the detection agent is a colloidal metal. The colloidal metal material may include water-insoluble metal particles or metallic compounds dispersed in a liquid, a hydrosol, or a metal sol. The colloidal metal may be selected from the metals in groups IA, IB, IIB and IIIB of the periodic table, as well as the transition metals, especially those of group VIII. Preferred metals include gold, silver, aluminum, ruthenium, zinc, iron, nickel and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the A13+, Ru3+, Zn2+, Fe3+, Ni2+ and Ca2+ ions.

When the RNA or DNA bridge is cut by the activated TnpB polypeptide, the aforementioned color shift is observed. In certain example embodiments the particles are colloidal metals. In certain other example embodiments, the colloidal metal is a colloidal gold. In certain example embodiments, the colloidal nanoparticles are 15 nm gold nanoparticles (AuNPs). Due to the unique surface properties of colloidal gold nanoparticles, maximal absorbance is observed at 520 nm when fully dispersed in solution and appear red in color to the naked eye. Upon aggregation of AuNPs, they exhibit a red-shift in maximal absorbance and appear darker in color, eventually precipitating from solution as a dark purple aggregate. In certain example embodiments the nanoparticles are modified to include DNA linkers extending from the surface of the nanoparticle. Individual particles are linked together by single-stranded RNA (ssRNA) or single-stranded DNA (ssDNA) bridges that hybridize on each end to at least a portion of the DNA linkers. Thus, the nanoparticles will form a web of linked particles and aggregate, appearing as a dark precipitate. Upon activation of the TnpB polypeptides disclosed herein, the ssRNA or ssDNA bridge will be cleaved, releasing the AU NPS from the linked mesh and produce a visible red color. Example DNA linkers and bridge sequences are listed below. Thiol linkers on the end of the DNA linkers may be used for surface conjugation to the AuNPS. Other forms of conjugation may be used. In certain example embodiments, two populations of AuNPs may be generated, one for each DNA linker. This will help facilitate proper binding of the ssRNA bridge with proper orientation. In certain example embodiments, a first DNA linker is conjugated by the 3′ end while a second DNA linker is conjugated by the 5′ end.

In certain other example embodiments, the masking construct may comprise an RNA or DNA oligonucleotide to which are attached a detectable label and a masking agent of that detectable label. An example of such a detectable label/masking agent pair is a fluorophore and a quencher of the fluorophore. Quenching of the fluorophore can occur as a result of the formation of a non-fluorescent complex between the fluorophore and another fluorophore or non-fluorescent molecule. This mechanism is known as ground-state complex formation, static quenching, or contact quenching. Accordingly, the RNA or DNA oligonucleotide may be designed so that the fluorophore and quencher are in sufficient proximity for contact quenching to occur. Fluorophores and their cognate quenchers are known in the art and can be selected for this purpose by one having ordinary skill in the art. The particular fluorophore/quencher pair is not critical in the context of this invention, only that selection of the fluorophore/quencher pairs ensures masking of the fluorophore. Upon activation of the effector proteins disclosed herein, the RNA or DNA oligonucleotide is cleaved thereby severing the proximity between the fluorophore and quencher needed to maintain the contact quenching effect. Accordingly, detection of the fluorophore may be used to determine the presence of a target molecule in a sample.

In certain other example embodiments, the masking construct may comprise one or more RNA oligonucleotides to which are attached one or more metal nanoparticles, such as gold nanoparticles. In one embodiment, the masking construct comprises a plurality of metal nanoparticles crosslinked by a plurality of RNA or DNA oligonucleotides forming a closed loop. In one embodiment, the masking construct comprises three gold nanoparticles crosslinked by three RNA or DNA oligonucleotides forming a closed loop. In one embodiment, the cleavage of the RNA or DNA oligonucleotides by the TnpB protein leads to a detectable signal produced by the metal nanoparticles.

In certain other example embodiments, the masking construct may comprise one or more RNA or DNA oligonucleotides to which are attached one or more quantum dots. In one embodiment, the cleavage of the RNA or DNA oligonucleotides by the TnpB protein leads to a detectable signal produced by the quantum dots.

In one example embodiment, the masking construct may comprise a quantum dot. The quantum dot may have multiple linker molecules attached to the surface. At least a portion of the linker molecule comprises RNA or DNA. The linker molecule is attached to the quantum dot at one end and to one or more quenchers along the length or at terminal ends of the linker such that the quenchers are maintained in sufficient proximity for quenching of the quantum dot to occur. The linker may be branched. As above, the quantum dot/quencher pair is not critical, only that selection of the quantum dot/quencher pair ensures masking of the fluorophore. Quantum dots and their cognate quenchers are known in the art and can be selected for this purpose by one having ordinary skill in the art. Upon activation of the effector proteins disclosed herein, the RNA or DNA portion of the linker molecule is cleaved thereby eliminating the proximity between the quantum dot and one or more quenchers needed to maintain the quenching effect. In certain example embodiments the quantum dot is streptavidin conjugated. RNA or DNA are attached via biotin linkers and recruit quenching molecules with the sequences/5Biosg/UCUCGUACGUUC/3IAbRQSp/ (SEQ ID NO: 238) or /5Biosg/UCUCGUACGUUCUCUCGUACGUUC/3IAbRQSp/ (SEQ ID NO: 239) where /5Biosg/ is a biotin tag and/31AbRQSp/is an Iowa black quencher (Iowa Black FQ). Upon cleavage, by the activated effectors disclosed herein the quantum dot will fluoresce visibly.

In specific embodiments, the detectable ligand may be a fluorophore and the masking component may be a quencher molecule.

In a similar fashion, fluorescence energy transfer (FRET) may be used to generate a detectable positive signal. FRET is a non-radiative process by which a photon from an energetically excited fluorophore (i.e., “donor fluorophore”) raises the energy state of an electron in another molecule (i.e., “the acceptor”) to higher vibrational levels of the excited singlet state. The donor fluorophore returns to the ground state without emitting a fluoresce characteristic of that fluorophore. The acceptor can be another fluorophore or non-fluorescent molecule. If the acceptor is a fluorophore, the transferred energy is emitted as fluorescence characteristic of that fluorophore. If the acceptor is a non-fluorescent molecule the absorbed energy is loss as heat. Thus, in the context of the embodiments disclosed herein, the fluorophore/quencher pair is replaced with a donor fluorophore/acceptor pair attached to the oligonucleotide molecule. When intact, the masking construct generates a first signal (negative detectable signal) as detected by the fluorescence or heat emitted from the acceptor. Upon activation of the effector proteins disclosed herein the RNA oligonucleotide is cleaved and FRET is disrupted such that fluorescence of the donor fluorophore is now detected (positive detectable signal).

In certain example embodiments, the masking construct comprises the use of intercalating dyes which change their absorbance in response to cleavage of long RNAs or DNAs to short nucleotides. Several such dyes exist. For example, pyronine-Y will complex with RNA and form a complex that has an absorbance at 572 nm. Cleavage of the RNA results in loss of absorbance and a color change. Methylene blue may be used in a similar fashion, with changes in absorbance at 688 nm upon RNA cleavage. Accordingly, in certain example embodiments the masking construct comprises an RNA and intercalating dye complex that changes absorbance upon the cleavage of RNA by the effector proteins disclosed herein.

In certain example embodiments, the masking construct may comprise an initiator for an HCR reaction. See e.g., Dirks and Pierce. PNAS 101, 15275-15728 (2004). HCR reactions utilize the potential energy in two hairpin species. When a single-stranded initiator having a portion of complementary to a corresponding region on one of the hairpins is released into the previously stable mixture, it opens a hairpin of one speces. This process, in turn, exposes a single-stranded region that opens a hairpin of the other species. This process, in turn, exposes a single stranded region identical to the original initiator. The resulting chain reaction may lead to the formation of a nicked double helix that grows until the hairpin supply is exhausted. Detection of the resulting products may be done on a gel or colorimetrically. Example colorimetric detection methods include, for example, those disclosed in Lu et al. “Ultra-sensitive colorimetric assay system based on the hybridization chain reaction-triggered enzyme cascade amplification ACS Appl Mater Interfaces, 2017, 9(1): 167-175, Wang et al. “An enzyme-free colorimetric assay using hybridization chain reaction amplification and split aptamers” Analyst 2015, 150, 7657-7662, and Song et al. “Non-covalent fluorescent labeling of hairpin DNA probe coupled with hybridization chain reaction for sensitive DNA detection.” Applied Spectroscopy, 70(4): 686-694(2016).

In certain example embodiments, the masking construct suppresses generation of a detectable positive signal until cleaved, or modified by an activated TnpB protein. In one embodiment, the masking construct may suppress generation of a detectable positive signal by masking the detectable positive signal, or generating a detectable negative signal instead.

### Amplification Reagents

In certain example embodiments, target RNAs and/or DNAs may be amplified prior to activating the CRISPR effector protein. Any suitable RNA or DNA amplification technique may be used. In certain example embodiments, the RNA or DNA amplification is an isothermal amplification. In certain example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR, multiple displacement amplification (MDA), rolling circle amplification (RCA), ligase chain reaction (LCR), or ramification amplification method (RAM).

In certain example embodiments, the RNA or DNA amplification is NASBA, which is initiated with reverse transcription of target RNA by a sequence-specific reverse primer to create an RNA/DNA duplex. RNase H is then used to degrade the RNA template, allowing a forward primer containing a promoter, such as the T7 promoter, to bind and initiate elongation of the complementary strand, generating a double-stranded DNA product. The RNA polymerase promoter-mediated transcription of the DNA template then creates copies of the target RNA sequence. Importantly, each of the new target RNAs can be detected by the guide RNAs thus further enhancing the sensitivity of the assay. Binding of the target RNAs by the guide RNAs then leads to activation of the CRISPR effector protein and the methods proceed as outlined above. The NASBA reaction has the additional advantage of being able to proceed under moderate isothermal conditions, for example at approximately 41° C., making it suitable for systems and devices deployed for early and direct detection in the field and far from clinical laboratories.

In certain other example embodiments, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, an RNA polymerase promoter, such as a T7 promoter, is added to one of the primers. This results in an amplified double-stranded DNA product comprising the target sequence and a RNA polymerase promoter. After, or during, the RPA reaction, an RNA polymerase is added that will produce RNA from the double-stranded DNA templates. The amplified target RNA can then in turn be detected by the CRISPR effector system. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above.

In an embodiment of the invention may comprise nickase-based amplification. The nicking enzyme may be a CRISPR protein. Accordingly, the introduction of nicks into dsDNA can be programmable and sequence-specific. FIG. 115 depicts an embodiment of the invention, which starts with two guides designed to target opposite strands of a dsDNA target. According to the invention, the nickase can be Cpf1, C2c1, Cas9 or any ortholog or CRISPR protein that cleaves or is engineered to cleave a single strand of a DNA duplex. The nicked strands may then be extended by a polymerase. In an embodiment, the locations of the nicks are selected such that extension of the strands by a polymerase is towards the central portion of the target duplex DNA between the nick sites. In one embodiment, primers are included in the reaction capable of hybridizing to the extended strands followed by further polymerase extension of the primers to regenerate two dsDNA pieces: a first dsDNA that includes the first strand Cpf1 guide site or both the first and second strand Cpf1 guide sites, and a second dsDNA that includes the second strand Cpf1 guide site or both the first and second strand Cprf guide sites. These pieces continue to be nicked and extended in a cyclic reaction that exponentially amplifies the region of the target between nicking sites.

The amplification can be isothermal and selected for temperature. In one embodiment, the amplification proceeds rapidly at 37 degrees. In other embodiments, the temperature of the isothermal amplification may be chosen by selecting a polymerase (e.g., Bsu, Bst, Phi29, klenow fragment etc.) operable at a different temperature.

Thus, wherease nicking isothermal amplification techniques use nicking enyzmes with fixed sequence preference (e.g., in nicking enzyme amplification reaction or NEAR), which requires denaturing of the original dsDNA target to allow annealing and extension of primers that add the nicking substrate to the ends of the target, use of a CRISPR nickase wherein the nicking sites can be programed via guide RNAs means that no denaturing step is necessary, enabling the entire reaction to be truly isothermal. This also simplifies the reaction because these primers that add the nicking substrate are different than the primers that are used later in the reaction, meaning that NEAR requires two primer sets (i.e., 4 primers) while Cpf1 nicking amplification only requires one primer set (i.e., two primers). This makes nicking Cpf1 amplification much simpler and easier to operate without complicated instrumentation to perform the denaturation and then cooling to the isothermal temperature.

Accordingly, in certain example embodiments the systems disclosed herein may include amplification reagents. Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present invention.

Other components of a biological or chemical reaction may include a cell lysis component in order to break open or lyse a cell for analysis of the materials therein. A cell lysis component may include, but is not limited to, a detergent, a salt as described above, such as NaCl, KCl, ammonium sulfate [(NH4)2SO4], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application, and may be specific to the reaction in some cases. Amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the invention may be any specific or general polymerase known in the art and useful or the invention, including Taq polymerase, Q5 polymerase, or the like.

In some embodiments, amplification reagents as described herein may be appropriate for use in hot-start amplification. Hot start amplification may be beneficial in some embodiments to reduce or eliminate dimerization of adaptor molecules or oligos, or to otherwise prevent unwanted amplification products or artifacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or aptamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents.

Amplification of nucleic acids may be performed using specific thermal cycle machinery or equipment, and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reactions conditions for the particular application or materials. One of skill in the art will understand and be able to optimize reaction conditions to obtain sufficient amplification.

In one embodiment, detection of DNA with the methods or systems of the invention requires transcription of the (amplified) DNA into RNA prior to detection.

It will be evident that detection methods of the invention can involve nucleic acid amplification and detection procedures in various combinations. The nucleic acid to be detected can be any naturally occurring or synthetic nucleic acid, including but not limited to DNA and RNA, which may be amplified by any suitable method to provide an intermediate product that can be detected. Detection of the intermediate product can be by any suitable method including but not limited to binding and activation of a CRISPR protein which produces a detectable signal moiety by direct or collateral activity.

### LAMP-Based Isothermal Amplification

In certain example embodiments, the LAMP amplification reagents may include primers to SARS-COV2. LAMP reagents may further comprise colorimetric and/or fluorescent detection reagents, such as hydroxy napthol blue (see, e.g., Goto, M., et al., Colorimetric detection of loop-mediated isothermal amplification reaction by using hydroxy naphthol blue. Biotechniques, 2009. 46(3): p. 167-72.) leuco triphenylmethane dyes (see, e.g., Miyamoto, S., et al., Method for colorimetric detection of double-stranded nucleic acid using leuco triphenylmethane dyes. Anal Biochem, 2015. 473: p. 28-33) and pH-snesitive dyes (see, e.g., Tanner, N. A., Y. Zhang, and T. C. Evans, Jr., Visual detection of isothermal nucleic acid amplification using pH-sensitive dyes. Biotechniques, 2015. 58(2): p. 59-68); as well as fluorescent detection (see, e.g., Yu et al., Clinical Chemistry, hvaa 102, doi: 10.1093/clinchem/hvaa102 12 May 2020), including use of quenching probes (see, e.g., Shirato et al., J Virol Methods. 2018 August; 258:41-48. doi: 10.1016/j.jviromet.2018.05.006). An overview of LAMP methods, including OSD-LAMP, for sequence-specific detection is described in Becherer et al., Anal. Methods, 2020,12, 717-746, doi: 10.1039/C9AY02246E, incorporated herein by reference.

In embodiments, the LAMP amplification reagents can comprise oligonucleotide strand displacement (OSD) probes. As used herein, oligonucleotide strand displacement probes are also referred to herein as oligonucleotide strand exchange probes or one-step strand displacement probes. The general concept of the use of OSD exchange is depicted in FIG. 1 of Bhadra et al., High-surety isothermal amplification and detection of SARS-COV-2, including with crude enzymes, doi: 10.1101/2020.04.13.039941. OSD probes rely on the binding enthalpy between the target-binding probe and amplicon of the LAMP reaction yielding a strand exchange reaction, leading to an easily read change in fluorescent signal. As a result, the results of a LAMP reaction can be visually or optically read fluorogenic OSD probes.

In an aspect, the OSD probes comprise a sequence specific for a target molecule. The OSD probes may comprise a pre-hybridized nucleic acid sequence, strand wherein the target sequence is 5, 6, 7, 8, 9, 10, 11, 12 or 13 nucleotides longer than the strand to which it is hybridized, allowing for sequence-specific interaction with a complementary target, with the OSD undergoing strand exchange and yielding a change in fluorescent signal.

In an aspect, the OSD probes are provided at a concentration of about 50 nM to 200 nM, about 75 nM to 150 nM, less than or equal to 200 nM, 190 nM, 180 nM, 170 nM, 160 nM, 150 nM, 140 nM, 130 nM, 120 nM, 110 nM, 100 nM, 90 nM, 80 nM, 75 nM, 65 nM, or 50 nM. Probes can be designed to be complementary to the loop region between the F1c and F2 primer binding sites for the LAMP primers, this can be referred to as the long toehold region. The complementary portion can be between about 9 and 14 nucleotides long, more preferably 11-12 nucleotides long. In an aspect, the longer strand of the OSD is labeled with a fluorescent molecule at the 5′ or 3′ end of the strand. In an aspect, the label is provided on the end opposite the designed complementary target region (long toehold region). The short strand is prepared with a quencher on one end of the probe, and can be designed to comprise a region complementary to a portion of the long strand. The OSD probes can be provided as part of LAMP reagents as described herein, which may comprise their use on any of the devices, cartridges or in any of the compositions as provided herein, including being provided as a lyophilized reagent in some instances.

### Extraction Solutions

In certain aspects, embodiments disclosed herein are directed to compositions and kits that consolidate extraction-free lysis and amplification of target nucleic acids into a single reaction volume. In certain example embodiments, the extraction-free lysis reagents can be used to extract nucleic acids from cells and/or viral particles. In contrast to existing protocols, the extraction-free lysis solution does not require isolation of the nucleic acid prior to further amplification. The extraction-free lysis reagents may be mixed with amplification reagents such as standard RT-PCR amplification reactions.

In one embodiment, extraction-free lysis solution and isothermal amplification reagents may be lyophilized in a single reaction volume, to be reconstituted by addition of a sample to be assayed. In certain other embodiments, the extraction-free lysis solution and isothermal amplification reagents may be lyophilized and stored on a cartridge or lateral flow strip, as discussed in further detail below.

In certain example embodiments, the single lysis reaction compositions and kits may further comprise one or more TnpB proteins possessing collateral activity and a detection construct. Pairing with one or more TnpB proteins may increase sensitivity or specificity of the assay. In certain example embodiments, the one or more TnpB proteins may be thermostable TnpB proteins. Example TnpB proteins are disclosed in further detail below.

In certain example embodiments, the single lysis amplification reaction compositions and kits may comprise optimized primers and/or one or more additives. In an aspect, the design optimizes the primers used in the amplification, in particular aspects, the isothermal amplification is used alone. In another aspect, the isothermal amplification is used with TnpB systems. In either approach, design considerations can follow a rational design for optimization of the reactions. In an example, varying additives with specific primers, target, TnpB protein, temperature, and other additive concentrations within the reaction can be identified. Optimization can be made with the goal of reducing the number of steps and buffer exchanges that have to occur in the reaction, simplifying the reaction and reducing the risks of contamination at transfer steps. In an aspect, addition of inhibitors, such as proteinase K can be considered so that buffer exchanges can be reduced. Similarly, optimizing the salt levels as well as the type of salt utilized can further facilitate and optimize the one-pot detections disclosed herein. In an aspect, potassium chloride can be utilized rather than sodium chloride when such amplification approaches are used with bead concentration in a lysis step.

In one embodiment, the compositions and kits may further comprise nucleic acid binding bead. The bead may be used to capture, concentrate or otherwise enrich for particular material. The bead may be magnetic, and may be provided to capture nucleic acid material. In another aspect, the bead is a silica bead. Beads may be utilized in an extraction step of the methods disclosed herein. Beads can be optionally used with the methods described herein, including with the one-pot methods that allow for concentration of viral nucleic acids from large volume samples, such as saliva or swab samples to allow for a single one-pot reaction method. Concentration of desired target molecules can be increased by about 10-fold, 50-fold, 100-fold, 200-fold, 500-fold, 800-fold, 1000-fold, 1500-fold, 2000-fold, 2500-fold, 3000-fold, or more.

Magnetic beads in a PEG and salt solution are preferred in an aspect, and in embodiments bind to viral RNA and/or DNA which allows for concentration and lysis concurrently. Silica beads can be used in another aspect. Capture moieties such as oligonucleotide functionalized beads are envisioned for use. The beads may be using with the extraction reagents, allowed to incubate with a sample and the lysis/extraction buffer, thereby concrrating target molecules on the beads. When used with a cartridge device detailed elsewhere herein, a magnet can be activated and the beads collected, with optional flushing of the extraction buffer and one or more washes performed. Advantageously, the beads can be used in the one-pot methods and systems without additional washings of the beads, allowing for a more efficient process without increased risks of contamination in multi-step processes. Beads can be utilized with the isothermal amplifications detailed herein, and the beads can flow into an amplification chamber of the cartridge or be maintained in the pot for the amplification step. Upon heating, nucleic acid can be released off the beads.

### Diagnostic Devices

The systems described herein can be embodied on diagnostic devices. A number of substrates and configurations may be used. The devices may be capable of defining multiple individual discrete volumes within the device. As used herein an “individual discrete volume” refers to a discrete space, such as a container, receptacle, or other defined volume or space that can be defined by properties that prevent and/or inhibit migration of target molecules, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof that can contain a sample within a defined space. Individual discrete volumes may be identified by molecular tags, such as nucleic acid barcodes. By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the use of non-walled, or semipermeable discrete volumes is that some reagents, such as buffers, chemical activators, or other agents may be passed through the discrete volume, while other materials, such as target molecules, may be maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In one embodiment, the compartment is an aqueous droplet in a water-in-oil emulsion. In specific embodiments, any of the applications, methods, or systems described herein requiring exact or uniform volumes may employ the use of an acoustic liquid dispenser.

In some embodiments, the individual discrete volumes may be droplets.

In certain example embodiments, the device comprises a flexible material substrate on which a number of spots may be defined. Flexible substrate materials suitable for use in diagnostics and biosensing are known within the art. The flexible substrate materials may be made of plant derived fibers, such as cellulosic fibers, or may be made from flexible polymers such as flexible polyester films and other polymer types. Within each defined spot, reagents of the system described herein are applied to the individual spots. Each spot may contain the same reagents except for a different guide RNA or set of guide RNAs, or where applicable, a different detection aptamer to screen for multiple targets at once. Thus, the systems and devices herein may be able to screen samples from multiple sources (e.g., multiple clinical samples from different individuals) for the presence of the same target, or a limited number of targets, or aliquots of a single sample (or multiple samples from the same source) for the presence of multiple different targets in the sample. In certain example embodiments, the elements of the systems described herein are freeze dried onto the paper or cloth substrate. Example flexible material based substrates that may be used in certain example devices are disclosed in Pardee et al. Cell. 2016, 165(5): 1255-66 and Pardee et al. Cell. 2014, 159(4): 950-54. Suitable flexible material-based substrates for use with biological fluids, including blood are disclosed in International Patent Application Publication No. WO/2013/071301 entitled “Paper based diagnostic test” to Shevkoplyas et al. U.S. Patent Application Publication No. 2011/0111517 entitled “Paper-based microfluidic systems” to Siegel et al. and Shafiee et al. “Paper and Flexible Substrates as Materials for Biosensing Platforms to Detect Multiple Biotargets” Scientific Reports 5:8719(2015). Further flexible based materials, including those suitable for use in wearable diagnostic devices are disclosed in Wang et al. “Flexible Substrate-Based Devices for Point-of-Care Diagnostics” Cell 34(11): 909-21(2016). Further flexible based materials may include nitrocellulose, polycarbonate, methylethyl cellulose, polyvinylidene fluoride (PVDF), polystyrene, or glass (see e.g., US20120238008). In one embodiment, discrete volumes are separated by a hydrophobic surface, such as but not limited to wax, photoresist, or solid ink.

In some embodiments, a dosimeter or badge may be provided that serves as a sensor or indicator such that the wearer is notified of exposure to certain microbes or other agents. For example, the systems described herein may be used to detect a particular pathogen. Likewise, aptamer based embodiments disclosed above may be used to detect both polypeptide as well as other agents, such as chemical agents, to which a specific aptamer may bind. Such a device may be useful for surveillance of soldiers or other military personnel, as well as clinicians, researchers, hospital staff, and the like, in order to provide information relating to exposure to potentially dangerous agents as quickly as possible, for example for biological or chemical warfare agent detection. In other embodiments, such a surveillance badge may be used for preventing exposure to dangerous microbes or pathogens in immunocompromised patients, burn patients, patients undergoing chemotherapy, children, or elderly individuals.

In specific embodiments, each individual discrete volume further comprises one or more detection aptamers comprising a masked RNA polymerase promoter binding site or a masked primer binding site. As such, each individual discrete volume may further comprise nucleic acid amplification reagents.

In specific embodiments, the target molecule may be a target DNA and the individual discrete volumes further comprise a primer that binds the target DNA and comprises an RNA polymerase promoter.

Samples sources that may be analyzed using the systems and devices described herein include biological samples of a subject or environmental samples. Environmental samples may include surfaces or fluids. The biological samples may include, but are not limited to, saliva, blood, plasma, sera, stool, urine, sputum, mucous, lymph, synovial fluid, spinal fluid, cerebrospinal fluid, a swab from skin or a mucosal membrane, or combination thereof. In an example embodiment, the environmental sample is taken from a solid surface, such as a surface used in the preparation of food or other sensitive compositions and materials.

In other example embodiments, the elements of the systems described herein may be place on a single use substrate, such as swab or cloth that is used to swab a surface or sample fluid. For example, the system could be used to test for the presence of a pathogen on a food by swabbing the surface of a food product, such as a fruit or vegetable. Similarly, the single use substrate may be used to swab other surfaces for detection of certain microbes or agents, such as for use in security screening. Single use substrates may also have applications in forensics, where the CRISPR systems are designed to detect, for example identifying DNA SNPs that may be used to identify a suspect, or certain tissue or cell markers to determine the type of biological matter present in a sample. Likewise, the single use substrate could be used to collect a sample from a patient-such as a saliva sample from the mouth- or a swab of the skin. In other embodiments, a sample or swab may be taken of a meat product on order to detect the presence of absence of contaminants on or within the meat product.

Near-real-time microbial diagnostics are needed for food, clinical, industrial, and other environmental settings (see e.g., Lu T K, Bowers J, and Koeris M S., Trends Biotechnol. 2013 June; 31(6): 325-7). In one embodiment, the present invention is used for rapid detection of foodborne pathogens using guide RNAs specific to a pathogen (e.g., Campylobacter jejuni, Clostridium perfringens, Salmonella spp., Escherichia coli, Bacillus cereus, Listeria monocytogenes, Shigella spp., Staphylococcus aureus, Staphylococcal enteritis, Streptococcus, Vibrio cholerae, Vibrio parahaemolyticus, Vibrio vulnificus, Yersinia enterocolitica and Yersinia pseudotuberculosis, Brucella spp., Corynebacterium ulcerans, Coxiella burnetii, or Plesiomonas shigelloides).

In one embodiment, the device is or comprises a flow strip. For instance, a lateral flow strip allows for detection by color. The reporter is modified to have a first molecule (such as for instance FITC) attached to the 5′ end and a second molecule (such as for instance biotin) attached to the 3′ end (or vice versa). The lateral flow strip is designed to have two capture lines with anti-first molecule (e.g., anti-FITC) antibodies hybridized at the first line and anti-second molecule (e.g., anti-biotin) antibodies at the second downstream line. As the reaction flows down the strip, uncleaved reporter will bind to anti-first molecule antibodies at the first capture line, while cleaved reporters will liberate the second molecule and allow second molecule binding at the second capture line. Second molecule sandwich antibodies, for instance conjugated to nanoparticles, such as gold nanoparticles, will bind any second molecule at the first or second line and result in a strong readout/signal (e.g., color). As more reporter is cleaved, more signal will accumulate at the second capture line and less signal will appear at the first line. In certain aspects, the invention relates to the use of a follow strip as described herein for detecting nucleic acids or polypeptides. In certain aspects, the invention relates to a method for detecting nucleic acids or polypeptides with a flow strip as defined herein, e.g., (lateral) flow tests or (lateral) flow immunochromatographic assays.

The embodiments disclosed herein are directed to lateral flow detection devices that comprise TnpB systems. The device may comprise a lateral flow substrate for detecting a TnpB collateral reaction. Substrates suitable for use in lateral flow assays are known in the art. These may include, but are not necessarily limited to, membranes or pads made of cellulose and/or glass fiber, polyesters, nitrocellulose, or absorbent pads (J Saudi Chem Soc 19(6): 689-705; 2015). The TnpB system, i.e., one or more TnpB systems and corresponding reporter constructs are added to the lateral flow substrate at a defined reagent portion of the lateral flow substrate, typically on one end of the lateral flow substrate. Reporting constructs used within the context of the present invention comprise a first molecule and a second molecule linked by anDNA linker. The lateral flow substrate further comprises a sample portion. The sample portion may be equivalent to, continuous with, or adjacent to the reagent portion. The lateral flow strip further comprises a first capture line, typically a horizontal line running across the device, but other configurations are possible. The first capture region is proximate to and on the same end of the lateral flow substrate as the sample loading portion. A first binding agent that specifically binds the first molecule of the reporter construct is fixed or otherwise immobilized to the first capture region. The second capture region is located towards the opposite end of the lateral flow substrate from the first binding region. A second binding agent is fixed or otherwise immobilized at the second capture region. The second binding agent specifically binds the second molecule of the reporter construct, or the second binding agent may bind a detectable ligand. For example, the detectable ligand may be a particle, such as a colloidal particle, that when it aggregates can be detected visually. The particle may be modified with an antibody that specifically binds the second molecule on the reporter construct. If the reporter construct is not cleaved it will facilitate accumulation of the detectable ligand at the first binding region. If the reporter construct is cleaved the detectable ligand is released to flow to the second binding region. In such an embodiment, the second binding agent is an agent capable of specifically or non-specifically binding the detectable ligand on the antibody on the detectable ligand. Examples of suitable binding agents for such an embodiment include, but are not limited to, protein A and protein G.

Lateral support substrates may be located within a housing (see for example, “Rapid Lateral Flow Test Strips” Merck Millipore 2013). The housing may comprise at least one opening for loading samples and a second single opening or separate openings that allow for reading of detectable signal generated at the first and second capture regions.

The TnpB system may be freeze-dried to the lateral flow substrate and packaged as a ready to use device, or the TnpB system may be added to the reagent portion of the lateral flow substrate at the time of using the device. Samples to be screened are loaded at the sample loading portion of the lateral flow substrate. The samples must be liquid samples or samples dissolved in an appropriate solvent, usually aqueous. The liquid sample reconstitutes the TnpB reagents such that a TnpB reaction can occur. The liquid sample begins to flow from the sample portion of the substrate towards the first and second capture regions. Intact reporter construct is bound at the first capture region by binding between the first binding agent and the first molecule. Likewise, the detection agent will begin to collect at the first binding region by binding to the second molecule on the intact reporter construct. If target molecule(s) are present in the sample, the TnpB collateral effect is activated. As activated TnpB comes into contact with the bound reporter construct, the reporter constructs are cleaved, releasing the second molecule to flow further down the lateral flow substrate towards the second binding region. The released second molecule is then captured at the second capture region by binding to the second binding agent, where additional detection agent may also accumulate by binding to the second molecule. Accordingly, if the target molecule(s) is not present in the sample, a detectable signal will appear at the first capture region, and if the target molecule(s) is present in the sample, a detectable signal will appear at the location of the second capture region.

Specific binding-integrating molecules comprise any members of binding pairs that can be used in the present invention. Such binding pairs are known to those skilled in the art and include, but are not limited to, antibody-antigen pairs, enzyme-substrate pairs, receptor-ligand pairs, and streptavidin-biotin. In addition to such known binding pairs, novel binding pairs may be specifically designed. A characteristic of binding pairs is the binding between the two members of the binding pair.

Oligonucleotide Linkers having molecules on either end may comprise DNA if the TnpB has DNA collateral activity. Oligonucleotide linkers may be single stranded or double stranded, and in one embodiment, they could contain both RNA and DNA regions. Oligonucleotide linkers may be of varying lengths, such as 5-10 nucleotides, 10-20 nucleotides, 20-50 nucleotides, or more.

In some embodiments, the polypeptide identifier elements include affinity tags, such as hemagglutinin (HA) tags, Myc tags, FLAG tags, V5 tags, chitin binding protein (CBP) tags, maltose-binding protein (MBP) tags, GST tags, poly-His tags, and fluorescent proteins (for example, green fluorescent protein (GFP), yellow fluorescent protein (YFP), cyan fluorescent protein (CFP), dsRed, mCherry, Kaede, Kindling, and derivatives thereof, FLAG tags, Myc tags, AU1 tags, T7 tags, OLLAS tags, Glu-Glu tags, VSV tags, or a combination thereof. Other Affinity tags are well known in the art. Such labels can be detected and/or isolated using methods known in the art (for example, by using specific binding agents, such as antibodies, that recognize a particular affinity tag). Such specific binding agents (for example, antibodies) can further contain, for example, detectable labels, such as isotope labels and/or nucleic acid barcodes such as those described herein.

In certain example embodiments, a lateral flow device comprises a lateral flow substrate comprising a first end for application of a sample. The first region is loaded with a detectable ligand, such as those disclosed herein, for example a gold nanoparticle. The gold nanoparticle may be modified with a first antibody, such as an anti-FITC antibody. The first region also comprises a detection construct. In one example embodiment, a DNA detection construct and a TnpB system as disclosed herein. In one example embodiment, and for purposes of further illustration, the DNA construct may comprise a FAM molecule on a first end of the detection construction and a biotin on a second end of the detection construct. Upstream of the flow of solution from the first end of the lateral flow substrate is a first test band. The test band may comprise a biotin ligand. Accordingly, when the DNA detection construct is present it its initial state, i.e. in the absence of target, the FAM molecule on the first end will bind the anti-FITC antibody on the gold nanoparticle, and the biotin on the second end of the DNA construct will bind the biotin ligand allowing for the detectable ligand to accumulate at the first test, generating a detectable signal. Generation of a detectable signal at the first band indicate the absence of the target ligand. In the presence of target, the TnpB complex forms and the TnpB is activated resulting in cleavage of the DNA detection construct. In the absence of intact DNA detection construct the colloidal gold will flow past the second strip. The lateral flow device may comprise a second band, upstream of the first band. The second band may comprise a molecule capable of binding the antibody-labeled colloidal gold molecule, for example an anti-rabbit antibody capable of binding a rabbit anti-FTIC antibody on the colloidal gold. Therefore, in the presence of one or more targets, the detectable ligand will accumulate at the second band, indicating the presence of the one or more targets in the sample.

In certain example embodiments, the device is a microfluidic device that generates and/or merges different droplets (i.e., individual discrete volumes). For example, a first set of droplets may be formed containing samples to be screened and a second set of droplets formed containing the elements of the systems described herein. The first and second set of droplets are then merged and then diagnostic methods as described herein are carried out on the merged droplet set. Microfluidic devices disclosed herein may be silicone-based chips and may be fabricated using a variety of techniques, including, but not limited to, hot embossing, molding of elastomers, injection molding, LIGA, soft lithography, silicon fabrication and related thin film processing techniques. Suitable materials for fabricating the microfluidic devices include, but are not limited to, cyclic olefin copolymer (COC), polycarbonate, poly(dimethylsiloxane) (PDMS), and poly(methylacrylate) (PMMA). In one embodiment, soft lithography in PDMS may be used to prepare the microfluidic devices. For example, a mold may be made using photolithography which defines the location of flow channels, valves, and filters within a substrate. The substrate material is poured into a mold and allowed to set to create a stamp. The stamp is then sealed to a solid support, such as but not limited to, glass. Due to the hydrophobic nature of some polymers, such as PDMS, which absorbs some proteins and may inhibit certain biological processes, a passivating agent may be necessary (Schoffner et al. Nucleic Acids Research, 1996, 24:375-379). Suitable passivating agents are known in the art and include, but are not limited to, silanes, parylene, n-Dodecyl-b-D-matoside (DDM), pluronic, Tween-20, other similar surfactants, polyethylene glycol (PEG), albumin, collagen, and other similar proteins and peptides.

In certain example embodiments, the system and/or device may be adapted for conversion to a flow-cytometry readout in or allow to all of sensitive and quantitative measurements of millions of cells in a single experiment and improve upon existing flow-based methods, such as the PrimeFlow assay. In certain example embodiments, cells may be cast in droplets containing unpolymerized gel monomer, which can then be cast into single-cell droplets suitable for analysis by flow cytometry. A detection construct comprising a fluorescent detectable label may be cast into the droplet comprising unpolymerized gel monomer. Upon polymerization of the gel monomer to form a bead within a droplet. Because gel polymerization is through free-radical formation, the fluorescent reporter becomes covalently bound to the gel. The detection construct may be further modified to comprise a linker, such as an amine. A quencher may be added post-gel formation and will bind via the linker to the reporter construct. Thus, the quencher is not bound to the gel and is free to diffuse away when the reporter is cleaved by the TnpB. Amplification of signal in droplet may be achieved by coupling the detection construct to a hybridization chain reaction (HCR initiator) amplification. DNA/RNA hybrid hairpins may be incorporated into the gel which may comprise a hairpin loop that has a RNase sensitive domain. By protecting a strand displacement toehold within a hairpin loop that has a RNase sensitive domain, HCR initiators may be selectively deprotected following cleavage of the hairpin loop by the TnpB system. Following deprotection of HCR initiators via toehold mediated strand displacement, fluorescent HCR monomers may be washed into the gel to enable signal amplification where the initiators are deprotected.

An example of microfluidic device that may be used in the context of the invention is described in Hour et al. “Direct Detection and drug-resistance profiling of bacteremias using inertial microfluidics” Lap Chip. 15(10): 2297-2307(2016).

In systems described herein, may further be incorporated into wearable medical devices that assess biological samples, such as biological fluids, of a subject outside the clinic setting and report the outcome of the assay remotely to a central server accessible by a medical care professional. The device may include the ability to self-sample blood, such as the devices disclosed in U.S. Patent Application Publication No. 2015/0342509 entitled “Needle-free Blood Draw to Peeters et al., U.S. Patent Application Publication No. 2015/0065821 entitled “Nanoparticle Phoresis” to Andrew Conrad.

In some embodiments, the individual discrete volumes are microwells.

In certain example embodiments, the device may comprise individual wells, such as microplate wells. The size of the microplate wells may be the size of standard 6, 24, 96, 384, 1536, 3456, or 9600 sized wells. In certain example embodiments, the elements of the systems described herein may be freeze dried and applied to the surface of the well prior to distribution and use.

The devices disclosed herein may further comprise inlet and outlet ports, or openings, which in turn may be connected to valves, tubes, channels, chambers, and syringes and/or pumps for the introduction and extraction of fluids into and from the device. The devices may be connected to fluid flow actuators that allow directional movement of fluids within the microfluidic device. Example actuators include, but are not limited to, syringe pumps, mechanically actuated recirculating pumps, electroosmotic pumps, bulbs, bellows, diaphragms, or bubbles intended to force movement of fluids. In certain example embodiments, the devices are connected to controllers with programmable valves that work together to move fluids through the device. In certain example embodiments, the devices are connected to the controllers discussed in further detail below. The devices may be connected to flow actuators, controllers, and sample loading devices by tubing that terminates in metal pins for insertion into inlet ports on the device.

As shown herein the elements of the system are stable when freeze dried, therefore embodiments that do not require a supporting device are also contemplated, i.e., the system may be applied to any surface or fluid that will support the reactions disclosed herein and allow for detection of a positive detectable signal from that surface or solution. In addition to freeze-drying, the systems may also be stably stored and utilized in a pelletized form. Polymers useful in forming suitable pelletized forms are known in the art.

In some embodiments, the individual discrete volumes are defined on a solid substrate. In some embodiments, the individual discrete volumes are spots defined on a substrate. In some embodiments, the substrate may be a flexible materials substrate, for example, including, but not limited to, a paper substrate, a fabric substrate, or a flexible polymer-based substrate. In specific embodiments, the flexible materials substrate is a paper substrate or a flexible polymer based substrate.

In one embodiment, the TnpB is bound to each discrete volume in the device. Each discrete volume may comprise a different ωRNA specific for a different target molecule. In one embodiment, a sample is exposed to a solid substrate comprising more than one discrete volume each comprising a ωRNA specific for a target molecule. Not being bound by a theory, each ωRNA will capture its target molecule from the sample and the sample does not need to be divided into separate assays. Thus, a valuable sample may be preserved. The effector protein may be a fusion protein comprising an affinity tag. Affinity tags are well known in the art (e.g., HA tag, Myc tag, Flag tag, His tag, biotin). The effector protein may be linked to a biotin molecule and the discrete volumes may comprise streptavidin. In other embodiments, the CRISPR effector protein is bound by an antibody specific for the effector protein. Methods of binding a CRISPR enzyme has been described previously (see, e.g., US20140356867A1).

The devices disclosed herein may also include elements of point of care (POC) devices known in the art for analyzing samples by other methods. See, for example St John and Price, “Existing and Emerging Technologies for Point-of-Care Testing” (Clin Biochem Rev. 2014 August; 35(3): 155-167).

The present invention may be used with a wireless lab-on-chip (LOC) diagnostic sensor system (see e.g., U.S. Pat. No. 9,470,699 “Diagnostic radio frequency identification sensors and applications thereof”). In one embodiment, the present invention is performed in a LOC controlled by a wireless device (e.g., a cell phone, a personal digital assistant (PDA), a tablet) and results are reported to said device.

Radio frequency identification (RFID) tag systems include an RFID tag that transmits data for reception by an RFID reader (also referred to as an interrogator). In a typical RFID system, individual objects (e.g., store merchandise) are equipped with a relatively small tag that contains a transponder. The transponder has a memory chip that is given a unique electronic product code. The RFID reader emits a signal activating the transponder within the tag through the use of a communication protocol. Accordingly, the RFID reader is capable of reading and writing data to the tag. Additionally, the RFID tag reader processes the data according to the RFID tag system application. Currently, there are passive and active type RFID tags. The passive type RFID tag does not contain an internal power source, but is powered by radio frequency signals received from the RFID reader. Alternatively, the active type RFID tag contains an internal power source that enables the active type RFID tag to possess greater transmission ranges and memory capacity. The use of a passive versus an active tag is dependent upon the particular application.

Lab-on-the chip technology is well described in the scientific literature and consists of multiple microfluidic channels, input or chemical wells. Reactions in wells can be measured using radio frequency identification (RFID) tag technology since conductive leads from RFID electronic chip can be linked directly to each of the test wells. An antenna can be printed or mounted in another layer of the electronic chip or directly on the back of the device. Furthermore, the leads, the antenna and the electronic chip can be embedded into the LOC chip, thereby preventing shorting of the electrodes or electronics. Since LOC allows complex sample separation and analyses, this technology allows LOC tests to be done independently of a complex or expensive reader. Rather a simple wireless device such as a cell phone or a PDA can be used. In one embodiment, the wireless device also controls the separation and control of the microfluidics channels for more complex LOC analyses. In one embodiment, a LED and other electronic measuring or sensing devices are included in the LOC-RFID chip. Not being bound by a theory, this technology is disposable and allows complex tests that require separation and mixing to be performed outside of a laboratory.

In preferred embodiments, the LOC may be a microfluidic device. The LOC may be a passive chip, wherein the chip is powered and controlled through a wireless device. In one embodiment, the LOC includes a microfluidic channel for holding reagents and a channel for introducing a sample. In one embodiment, a signal from the wireless device delivers power to the LOC and activates mixing of the sample and assay reagents. Specifically, in the case of the present invention, the system may include a masking agent, CRISPR effector protein, and guide RNAs specific for a target molecule. Upon activation of the LOC, the microfluidic device may mix the sample and assay reagents. Upon mixing, a sensor detects a signal and transmits the results to the wireless device. In one embodiment, the unmasking agent is a conductive RNA molecule. The conductive RNA molecule may be attached to the conductive material. Conductive molecules can be conductive nanoparticles, conductive proteins, metal particles that are attached to the protein or latex or other beads that are conductive. In one embodiment, if DNA or RNA is used then the conductive molecules can be attached directly to the matching DNA or RNA strands. The release of the conductive molecules may be detected across a sensor. The assay may be a one step process.

Since the electrical conductivity of the surface area can be measured precisely quantitative results are possible on the disposable wireless RFID electro-assays. Furthermore, the test area can be very small allowing for more tests to be done in a given area and therefore resulting in cost savings. In one embodiment, separate sensors each associated with a different CRISPR effector protein and guide RNA immobilized to a sensor are used to detect multiple target molecules. Not being bound by a theory, activation of different sensors may be distinguished by the wireless device.

In addition to the conductive methods described herein, other methods may be used that rely on RFID or Bluetooth as the basic low cost communication and power platform for a disposable RFID assay. For example, optical means may be used to assess the presence and level of a given target molecule. In one embodiment, an optical sensor detects unmasking of a fluorescent masking agent.

In one embodiment, the device of the present invention may include handheld portable devices for diagnostic reading of an assay (see e.g., Vashist et al., Commercial Smartphone-Based Devices and Smart Applications for Personalized Healthcare Monitoring and Management, Diagnostics 2014, 4(3), 104-128; mReader from Mobile Assay; and Holomic Rapid Diagnostic Test Reader).

As noted herein, certain embodiments allow detection via colorimetric change which has certain attendant benefits when embodiments are utilized in POC situations and or in resource poor environments where access to more complex detection equipment to readout the signal may be limited. However, portable embodiments disclosed herein may also be coupled with hand-held spectrophotometers that enable detection of signals outside the visible range. An example of a hand-held spectrophotometer device that may be used in combination with the present invention is described in Das et al. “Ultra-portable, wireless smartphone spectrophotometer for rapid, non-destructive testing of fruit ripeness.” Nature Scientific Reports. 2016, 6:32504, DOI: 10.1038/srep32504. Finally, in one embodiment utilizing quantum dot-based masking constructs, use of a hand held UV light, or other suitable device, may be successfully used to detect a signal owing to the near complete quantum yield provided by quantum dots.

### Methods for Detecting Nucleic Acids

The low cost and adaptability of the assay platform lends itself to a number of applications including (i) general RNA/DNA quantitation, (ii) rapid, multiplexed RNA/DNA and protein expression detection, and (iii) sensitive detection of target nucleic acids, peptides, and proteins in both clinical and environmental samples. Additionally, the systems disclosed herein may be adapted for detection of transcripts within biological settings, such as cells. Given the highly specific nature of the CRISPR effectors described herein, it may be possible to track allelic specific expression of transcripts or disease-associated mutations in live cells.

In some embodiments, methods include detecting target nucleic acids in samples, comprising distributing a sample or set of samples into one or more individual discrete volumes comprising a TnpB system as described hereinof. The sample or set of samples may then be incubated under conditions sufficient to allow binding of the one or more ωRNAs to one or more target molecules, and the TnpB protein may be activated via binding of the one or more ωRNA to the one or more target molecules, wherein activating the CRISPR effector protein results in modification of the detection construct such that a detectable positive signal is generated. The one or more detectable positive signals may then be detected, with detection indicating the presence of one or more target molecules in the sample.

In some embodiments, methods of the invention include detecting polypeptides in samples, comprising distributing a sample or set of samples into a set of individual discrete volumes comprising peptide detection aptamers and a TnpB as described herein. The sample or set of samples may then be incubated under conditions sufficient to allow binding of the peptide detection aptamers to the one or more target molecules, wherein binding of the aptamer to a corresponding target molecule exposes the RNA polymerase binding site or primer binding site resulting in generation of a trigger RNA. The TnpB may then be activated via binding of the one or more ωRNAs to the trigger RNA, wherein activating the TnpB protein results in modification of the detection construct such that a detectable positive signal is produced. The detectable positive signal may then be detected, with detection of the detectable positive signal indicating the presence of one or more target molecules in a sample.

In certain example embodiments, a single guide sequence specific to a single target is placed in separate volumes. Each volume may then receive a different sample or aliquot of the same sample. In certain example embodiments, multiple ωRNAs each to separate target may be placed in a single well such that multiple targets may be screened in a different well. In order to detect multiple ωRNA in a single volume, in certain example embodiments, multiple TnpB proteins with different specificities may be used.

In embodiments, different TnpB orthologs with different sequence specificities may be used. Cutting motifs may be used to take advantage of the sequence specificities of different orthologs. The detection construct can comprise a cutting motif preferentially cut by a given TnpB ortholog. A cutting motif sequence can be a particular nucleotide base, a repeat nucleotide base in a homopolymer, or a heteropolymer of bases. The cutting motif can be a dinucleotide sequence, a trinucleotide sequence or more complex motifs comprising 4, 5, 6, 7, 8, 9, or 10 nucleotide motifs. For example, one orthologue may preferentially cut A, while others preferentially cut C, G, U/T. Accordingly, detection constructs completely comprising, or comprised of a substantial portion, of a single nucleotide may be generated, each with a different fluorophore that can be detected at differing wavelengths. In this way up to four different targets may be screened in a single individual discrete volume. In certain other example embodiments, different orthologues with different nucleotide editing preferences may be used such as TnpBs in combination with a Cas13 or Cas12.

In addition to single base editing preferences, additional detection constructs can be designed based on other motif cutting preferences of TnpB, Cas12, and Cas13 orthologs. For example, Cas13 or Cas12 orthologs may preferentially cut a dinucleotide sequence, a trinucleotide sequence or more complex motifs comprising 4, 5, 6, 7, 8, 9, or 10 nucleotide motifs. As an example, LwaCas13a showed strong preference for a hexanucleotide motif sequences, with CcaCas13b showing strong preference for other hexanucleotide motifs. Thus, the upper bound for multiplex assays using the embodiments disclosed herein is primarily limited by the number of distinguishable detectable labels and the detection channels needed to detect them. In certain example embodiments, 2, 3, 4, 5, 6, 7, 8, 9, 10, 11, 12, 13, 14, 15, 16, 17, 18, 19, 20, 21, 22, 23, 24, 25, 25, 27, 28, 29, or 30 different targets are detected. Example methods for identifying such motifs are further disclosed in the Working Examples below.

In specific embodiments, the target molecule may be a target DNA and the method may further comprise binding the target DNA with a primer comprising an RNA polymerase site, as described herein.

In specific embodiments, the one or more ωRNA may be designed to detect a single nucleotide polymorphism in a target RNA or DNA, or a splice variant of an RNA transcript.

A sample for use with the invention may be a biological or environmental sample, such as a food sample (fresh fruits or vegetables, meats), a beverage sample, a paper surface, a fabric surface, a metal surface, a wood surface, a plastic surface, a soil sample, a freshwater sample, a wastewater sample, a saline water sample, exposure to atmospheric air or other gas sample, or a combination thereof. For example, household/commercial/industrial surfaces made of any materials including, but not limited to, metal, wood, plastic, rubber, or the like, may be swabbed and tested for contaminants. Soil samples may be tested for the presence of pathogenic bacteria or parasites, or other microbes, both for environmental purposes and/or for human, animal, or plant disease testing. Water samples such as freshwater samples, wastewater samples, or saline water samples can be evaluated for cleanliness and safety, and/or potability, to detect the presence of, for example, Cryptosporidium parvum, Giardia lamblia, or other microbial contamination. In further embodiments, a biological sample may be obtained from a source including, but not limited to, a tissue sample, saliva, blood, plasma, sera, stool, urine, sputum, mucous, lymph, synovial fluid, cerebrospinal fluid, ascites, pleural effusion, seroma, pus, bile, aqueous or vitreous humor, transudate, exudate, or swab of skin or a mucosal membrane surface. In some particular embodiments, an environmental sample or biological samples may be crude samples and/or the one or more target molecules may not be purified or amplified from the sample prior to application of the method. Identification of microbes may be useful and/or needed for any number of applications, and thus any type of sample from any source deemed appropriate by one of skill in the art may be used in accordance with the invention.

In some embodiments, the one or more ωRNAs may be designed to bind to cell free nucleic acids. In some embodiments, the one or more ωRNAs may be designed to detect a single nucleotide polymorphism in a target RNA or DNA, or a splice variant of an RNA transcript. In some embodiments, the one or more guide RNAs are designed to bind to one or more target molecules that are diagnostic for a disease state, as described herein.

In some embodiments, the disease state may be an infection, an organ disease, a blood disease, an immune system disease, a cancer, a brain and nervous system disease, an endocrine disease, a pregnancy or childbirth-related disease, an inherited disease, or an environmentally-acquired disease.

In certain example embodiments, the systems, devices, and methods, disclosed herein are directed to detecting the presence of one or more microbial agents in a sample, such as a biological sample obtained from a subject. In certain example embodiments, the microbe may be a bacterium, a fungus, a yeast, a protozoa, a parasite, or a virus. Accordingly, the methods disclosed herein can be adapted for use in other methods (or in combination) with other methods that require quick identification of microbe species, monitoring the presence of microbial proteins (antigens), antibodies, antibody genes, detection of certain phenotypes (e.g., bacterial resistance), monitoring of disease progression and/or outbreak, and antibiotic screening. Because of the rapid and sensitive diagnostic capabilities of the embodiments disclosed here, detection of microbe species type, down to a single nucleotide difference, and the ability to be deployed as a POC device, the embodiments disclosed herein may be used guide therapeutic regimens, such as selection of the appropriate antibiotic or antiviral. The embodiments disclosed herein may also be used to screen environmental samples (air, water, surfaces, food etc.) for the presence of microbial contamination.

Disclosed is a method to identify microbial species, such as bacterial, viral, fungal, yeast, or parasitic species, or the like. Particular embodiments disclosed herein describe methods and systems that will identify and distinguish microbial species within a single sample, or across multiple samples, allowing for recognition of many different microbes. The present methods allow the detection of pathogens and distinguishing between two or more species of one or more organisms, e.g., bacteria, viruses, yeast, protozoa, and fungi or a combination thereof, in a biological or environmental sample, by detecting the presence of a target nucleic acid sequence in the sample. A positive signal obtained from the sample indicates the presence of the microbe. Multiple microbes can be identified simultaneously using the methods and systems of the invention, by employing the use of more than one effector protein, wherein each effector protein targets a specific microbial target sequence. In this way, a multi-level analysis can be performed for a particular subject in which any number of microbes can be detected at once. In some embodiments, simultaneous detection of multiple microbes may be performed using a set of probes that can identify one or more microbial species.

Multiplex analysis of samples enables large-scale detection of samples, reducing the time and cost of analyses. However, multiplex analyses are often limited by the availability of a biological sample. In accordance with the invention, however, alternatives to multiplex analysis may be performed such that multiple effector proteins can be added to a single sample and each masking construct may be combined with a separate quencher dye. In this case, positive signals may be obtained from each quencher dye separately for multiple detection in a single sample.

Disclosed herein are methods for distinguishing between two or more species of one or more organisms in a sample. The methods are also amenable to detecting one or more species of one or more organisms in a sample.

In some embodiments, the methods provide for detection of disease states that are characterized by the presence or absence of an antibiotic or drug resistance or susceptibility gene or transcript or polypeptide, preferably in a pathogen or a cell.

### Devices for Detection Assays

In one embodiment, the detection assay can be provided on a cartridge or chip. In an aspect, the cartridge can comprise one or more ampoules and one or more wells that are communicatively coupled, allowing for the transfer, exchange or movement of reagents and sample with or without the use of beads through the chambers of the cartridge and facilitating detection assays utilizing systems/devices for facilitating the detection assay on the cartridge.

### Cartridge

The cartridge, also referred to herein as a chip, according to the present invention comprises a series of components of ampoules and chambers that are communicatively coupled with one or more other components on the cartridge. The coupling is typically a fluidic communication, for example, via channels. The cartridge may comprise a membrane that seals one or more of the chambers and/or ampoules. In an aspect, the membrane allows for storage of reagents, buffers and other solid or fluid components which cover and seal the cartridge. The membrane can be configured to be punctured, pierced or otherwise released from sealing or covering one or more components of the cartridge by a means for releasing reagents.

As noted above, certain embodiments enable the use of nucleic acid binding beads to concentrate target nucleic acid but that do not require elution of the isolated nucleic acid. Thus, in certain example embodiments, the cartridge may further comprise an activatable magnet, such as an electro-magnet. A means for activating the magnet may be located on the device, or the means for supplying the magnet or activating the magnet on the cartridge may be provided by a second device, such as those disclosed in further detail below.

### Ampoules

The ampoules, also referred to as blisters, allow for storage and release of reagents throughout the cartridge. Ampoules can include liquid or solid reagents, for example, lysis reagents in one ampoule and reaction reagents in another ampoule. The reagents can be as described elsewhere herein, and can be adapted for the use in the cartridge. The ampoule may be sealed by a film that allows for the bursting, puncture or other release of the contents of the ampoules. See, e.g., Becker, H. & Gärtner, C. Microfluidics-enabled diagnostic systems: markets, challenges, and examples. In Microchip Diagnostics: Methods and Protocols (eds Taly, V. et al.) (Springer, New York, 2017); Czurratis et al., doi: 10.1088/0960-1317/25/4/045002. Considerations for ampoules can include as discussed in, for example, Smith, S., et al., Blister pouches for effective reagent storage on microfluidic chips for blood cell counting. Microfluid Nanofluid 20, 163(2016). DOI:10.1007/s10404-016-1830-2. In an aspect, the seal is a frangible seal formed of a composite-layer film that is assembled to the cartridge main body. While referred to herein as an ampoule, the ampoule may comprise a cavity on a chip which comprises a sealed film that is opened by the release means.

### Chambers

The chambers on the chip may located and sized for fluidic communication via channels or other communication means with ampoules and/or other chambers on the chip.

### Means for Reading the Results of the Assay

A means for reading the results of the assay can be provided in the system. The means for reading the results of the assay will depend in part on the type of detectable signal generated by the assay. In particular embodiments, the assay generates a detectable fluorescent or color readaout. In these instances, the means for reading the results of the assay will be an optic means, for example a single channel or multi-channel optical means such as a fluorimeter, colorimeter or other spectroscopic sensor.

A combination of means for reading the results of the assay can be utilized, and may include readings such as turbidity, temperature, magnetic, radio, or electrical properties and or optical properties, including scattering, polarization effects, etc.

The system may further comprise a user interface for programming the device and/or readout of the results of the assay. The user interface may comprise an LED screen. The system can be further configured for a USB port that can allow for docking of four or more devices.

In an aspect, the system comprises a means for activating a magnet that is disposed within or on the cartridge.

### Lateral Flow Devices

In one embodiment, the detection assay can be provided on a lateral flow device, see, e.g., International Publication WO 2019/071051, incorporated herein by reference for exemplary lateral flow devices. The lateral flow device can be adapted to detect one or more coronaviruses and/or other viruses in combination of the coronavirus. The lateral flow device may comprise a flexible substrate, such as a paper substrate or a flexible polymer-based substrate, which can include freeze-dried reagents for detection assays with a visual readout of the assay results. See, WO 2019/071051 at [0145]-[0151] and Example 2, specifically incorporated herein by reference. In an aspect, lyophilized reagents can include preferred excipients that aid in rate of reaction, specificity, or other variable, for example, trehalose, histidine, and/or glycine. In one embodiment, a coronavirus assay can be utilized with isothermal amplification reagents, allowing amplification without complex instrumentation that may be unavailable in the field. Accordingly, the assay can be adapted for field diagnostics, including use of visual readout on a lateral flow device, rapid, sensitive detection and can be deployed for early and direct detection. Colorimetric detection can be utilized and may be particularly suited for field deployable applications, as described in International Application PCT/US2019/015726, published as WO2019/148206. In particular, colorimetric detection can be as described in WO2019/148206 at FIGS. 102, 105, 107-111 and [00306]-[00324], incorporated herein by reference and may be utilized with the TnpB systems.

In one embodiment, the invention provides a lateral flow device comprising a substrate comprising a first end and a second end. The first end may comprise a sample loading portion, a first region comprising a detectable ligand, two or more TnpB systems, two or more detection constructs, and one or more first capture regions, each comprising a first binding agent. The substrate may also comprise two or more second capture regions between the first region of the first end and the second end, each second capture region comprising a different binding agent. Each of the two or more TnpB systems may comprise a TnpB protein and one or more nucleic acid component molecules, each nucleic acid component molecule sequence configured to bind one or more target molecules.

The device may comprise a lateral flow substrate for detecting a reaction between a TnpB polypeptide and a target molecule triggering collateral, non-specific cleavage of detection construct. Substrates suitable for use in lateral flow assays are known in the art. These may include, but are not necessarily limited to, membranes or pads made of cellulose and/or glass fiber, polyesters, nitrocellulose, or absorbent pads (J Saudi Chem Soc 19(6): 689-705; 2015), and other embodiments further described herein. The detection system, i.e., one or more TnpB systems and corresponding reporter constructs are added to the lateral flow substrate at a defined reagent portion of the lateral flow substrate, typically on one end of the lateral flow substrate. Reporting constructs used within the context of the present invention can comprise a first molecule and a second molecule linked by an RNA or DNA linker. The lateral flow substrate further comprises a sample portion. The sample portion may be equivalent to, continuous with, or adjacent to the reagent portion. In an aspect, the lateral flow substrate can be contained within a further device. In an aspect, the lateral flow substrate can be utilized for visual readout of a detectable signal in one-pot reactions, e.g., wherein steps of extracting, amplifying and detecting are performed in an individual discrete volume.

### Lateral Flow Substrate

In certain example embodiments, a lateral flow device comprises a lateral flow substrate on which detection can be performed. Substrates suitable for use in lateral flow assays are known in the art. These may include, but are not necessarily limited to, membranes or pads made of cellulose and/or glass fiber, polyesters, nitrocellulose, or absorbent pads (J Saudi Chem Soc 19(6): 689-705; 2015).

Lateral support substrates comprise a first and second end, and one or more capture regions that each comprise binding agents. The first end may comprise a sample loading portion, a first region comprising a detectable ligand, two or more TnpB systems, two or more detection constructs, and one or more first capture regions, each comprising a first binding agent. The substrate may also comprise two or more second capture regions between the first region of the first end and the second end, each second capture region comprising a different binding agent. Each of the two or more TnpB systems may comprise a TnpB protein and one or more nucleic acid component molecules, each nucleic acid component configured to bind one or more target molecules. The lateral flow substrates may be configured to detect a reaction wherein collateral, non-specific cleavage is triggered upon binding and cleavage of a target molecule in the reaction by the TnpB protein.

Lateral support substrates may be located within a housing (see for example, “Rapid Lateral Flow Test Strips” Merck Millipore 2013). The housing may comprise at least one opening for loading samples and a second single opening or separate openings that allow for reading of detectable signal generated at the first and second capture regions.

The embodiments disclosed herein can be prepared in freeze-dried format for convenient distribution and point-of-care (POC) applications. Such embodiments are useful in multiple scenarios in human health including, for example, viral detection, bacterial strain typing, sensitive genotyping, and detection of disease-associated cell free DNA. Accordingly, the lateral substrate comprising one or more of the elements of the system, including detectable ligands, TnpB systems, detection constructs and binding agents may be freeze-dried to the lateral flow substrate and packaged as a ready to use device. Alternatively, all or a portion of the elements of the system may be added to the reagent portion of the lateral flow substrate at the time of using the device.

### First End and Second End of the Substrate

The substrate of the lateral flow device comprises a first and second end. The TnpB system, i.e. one or more TnpB systems and corresponding reporter constructs are added to the lateral flow substrate at a defined reagent portion of the lateral flow substrate, typically on a first end of the lateral flow substrate. Reporting constructs used within the context of the present invention comprise a first molecule and a second molecule linked by an RNA or DNA linker. The lateral flow substrate further comprises a sample portion. The sample portion may be equivalent to, continuous with, or adjacent to the reagent portion.

In certain example embodiments, the first end comprises a first region. The first region comprises a detectable ligand, two or more TnpB systems, two or more detection constructs, and one or more first capture regions, each comprising a first binding agent.

### Capture Regions

The lateral flow substrate can comprise one or more capture regions. In embodiments the first end of the lateral flow substrate comprises one or more first capture regions, with two or more second capture regions between the first region of the first end of the substrate and the second end of the substrate. The capture regions may be provided as a capture line, typically a horizontal line running across the device, but other configurations are possible. The first capture region is proximate to and on the same end of the lateral flow substrate as the sample loading portion.

### Binding Agents

Specific binding-integrating molecules comprise any members of binding pairs that can be used in the present invention. Such binding pairs are known to those skilled in the art and include, but are not limited to, antibody-antigen pairs, enzyme-substrate pairs, receptor-ligand pairs, and streptavidin-biotin. In addition to such known binding pairs, novel binding pairs may be specifically designed. A characteristic of binding pairs is the binding between the two members of the binding pair.

A first binding agent that specifically binds the first molecule of the reporter construct is fixed or otherwise immobilized to the first capture region. The second capture region is located towards the opposite end of the lateral flow substrate from the first capture region. A second binding agent is fixed or otherwise immobilized at the second capture region. The second binding agent specifically binds the second molecule of the reporter construct, or the second binding agent may bind a detectable ligand. For example, the detectable ligand may be a particle, such as a colloidal particle, that when it aggregates can be detected visually, and generates a detectable positive signal. The particle may be modified with an antibody that specifically binds the second molecule on the reporter construct. If the reporter construct is not cleaved it will facilitate accumulation of the detectable ligand at the first binding region. If the reporter construct is cleaved the detectable ligand is released to flow to the second binding region. In such an embodiment, the second binding region comprises a second binding agent capable of specifically or non-specifically binding the detectable ligand on the antibody of the detectable ligand. Binding agents can be, for example, antibodies, that recognize a particular affinity tag. Such binding agents can further contain, for example, detectable labels, such as isotope labels and/or nucleic acid barcodes. A barcode is a short sequence of nucleotides (for example, DNA, RNA, or combinations thereof) that is used as an identifier. A nucleic acid barcode may have a length of 4-100 nucleotides and be either single or double-stranded. Methods for identifying cells with barcodes are known in the art. Accordingly, nucleic acid component molecules of the TnpB systems described herein may be used to detect the barcode.

### Detectable Ligands

The first region is loaded with a detectable ligand, such as those disclosed herein, for example a gold nanoparticle. The detectable ligand may be a particle, such as a colloidal particle, that when it aggregates can be detected visually. The particle may be modified with an antibody that specifically binds the second molecule on the reporter construct. If the reporter construct is not cleaved it will facilitate accumulation of the detectable ligand at the first binding region. If the reporter construct is cleaved the detectable ligand is released to flow to the second binding region. In such an embodiment, the second binding agent is an agent capable of specifically or non-specifically binding the detectable ligand on the antibody on the detectable ligand. Examples of suitable binding agents for such an embodiment include, but are not limited to, protein A and protein G. In some examples, the detectable ligand is a gold nanoparticle, which may be modified with a first antibody, such as an anti-FITC antibody.

### Lateral Flow Detection Constructs

The first region also comprises a detection construct. In one example embodiment, an RNA detection construct and a TnpB system (a TnpB protein and one or more nucleic acid component molecules configured to bind to one or more target sequences) as disclosed herein. In one example embodiment, and for purposes of further illustration, the RNA construct may comprise a FAM molecule on a first end of the detection construction and a biotin on a second end of the detection construct. Upstream of the flow of solution from the first end of the lateral flow substrate is a first test band. The test band may comprise a biotin ligand. Accordingly, when the RNA detection construct is present it its initial state, i.e., in the absence of target, the FAM molecule on the first end will bind the anti-FITC antibody on the gold nanoparticle, and the biotin on the second end of the RNA construct will bind the biotin ligand allowing for the detectable ligand to accumulate at the first test, generating a detectable signal. Generation of a detectable signal at the first band indicates the absence of the target ligand. In the presence of target, the TnpB complex forms and the TnpB protein is activated resulting in cleavage of the detection construct. In the absence of intact RNA detection construct the colloidal gold will flow past the second strip. The lateral flow device may comprise a second band, upstream of the first band. The second band may comprise a molecule capable of binding the antibody-labeled colloidal gold molecule, for example an anti-rabbit antibody capable of binding a rabbit anti-FITC antibody on the colloidal gold. Therefore, in the presence of one or more targets, the detectable ligand will accumulate at the second band, indicating the presence of the one or more targets in the sample.

In one embodiment, the first end of the lateral flow device comprises two detection constructs and each of the two detection constructs comprises an RNA or DNA oligonucleotide, comprising a first molecule on a first end and a second molecule on a second end. The first molecule and the second molecule may be linked by an RNA or DNA linker.

In one embodiment, the first molecule on the first end of the first detection construct may be FAM and the second molecule on the second end of the first detection construct may be biotin, or vice versa. In one embodiment, the first molecule on the first end of the second detection construct may be FAM and the second molecule on the second end of the second detection construct may be Digoxigenin (DIG), or vice versa.

In one embodiment, the first end may comprise three detection constructs, wherein each of the three detection constructs comprises an RNA or DNA oligonucleotide, comprising a first molecule on a first end and a second molecule on a second end. In specific embodiments, the first and second molecules on the detection constructs comprise Tye 665 and Alexa 488; Tye 665 and FAM, and Tye 665 and Digoxigenin (DIG), respectively.

In one embodiment, the first end of the lateral flow device comprises two or more TnpB systems, also referred to as a TnpB system. In one embodiment, such a TnpB system may include a TnpB protein and one or more nucleic acid component molecules configured to bind to one or more target sequences.

### Samples

When utilizing the detection systems with a lateral flow substrate, samples to be screened are loaded at the sample loading portion of the lateral flow substrate. The samples must be liquid samples or samples dissolved in an appropriate solvent, usually aqueous. The liquid sample reconstitutes the detection reagents such that a detection reaction can occur. The liquid sample begins to flow from the sample portion of the substrate towards the first and second capture regions.

A sample for use with the invention may be a biological or environmental sample, such as a surface sample, a fluid sample, or a food sample (fresh fruits or vegetables, meats). Food samples may include a beverage sample, a paper surface, a fabric surface, a metal surface, a wood surface, a plastic surface, a soil sample, a freshwater sample, a wastewater sample, a saline water sample, exposure to atmospheric air or other gas sample, or a combination thereof. For example, household/commercial/industrial surfaces made of any materials including, but not limited to, metal, wood, plastic, rubber, or the like, may be swabbed and tested for contaminants. Soil samples may be tested for the presence of pathogenic bacteria or parasites, or other microbes, both for environmental purposes and/or for human, animal, or plant disease testing. Water samples such as freshwater samples, wastewater samples, or saline water samples can be evaluated for cleanliness and safety, and/or potability, to detect the presence of, for example, Cryptosporidium parvum, Giardia lamblia, or other microbial contamination. In further embodiments, a biological sample may be obtained from a source including, but not limited to, a tissue sample, saliva, blood, plasma, sera, stool, urine, sputum, mucous, lymph, synovial fluid, spinal fluid, cerebrospinal fluid, ascites, pleural effusion, seroma, pus, bile, aqueous or vitreous humor, transudate, exudate, or swab of skin or a mucosal membrane surface. In some particular embodiments, an environmental sample or biological samples may be crude samples and/or the one or more target molecules may not be purified or amplified from the sample prior to application of the method. Identification of microbes may be useful and/or needed for any number of applications, and thus any type of sample from any source deemed appropriate by one of skill in the art may be used in accordance with the invention.

In particular embodiments, the methods and systems can be utilized for direct detection from patient samples. In an aspect, the methods and systems can further allow for direct detection from patient samples with a visual readout to further facilitate field-deployability. In an aspect, a field depoloyable version can include, for example the lateral flow devices and systems as described herein, and/or colorimetric detection. The methods and systems can be utilized to distinguish multipe viral species and strains and identify clinically relevant mutations, important with viral outbreaks such as the coronavirus outbreak in Wuhan (2019-nCOV). In an aspect, the sample is from a nasophyringeal swab or a saliva sample. See., e.g., Wyllie et al., “Saliva is more sensitive for SARS-COV-2 detection in COVID-19 patients than nasopharyngeal swabs,” DOI: 10.1101/2020.04.16.20067835.

Methods for Detecting and/or Quantifying Target Nucleic Acids

In one embodiment, the invention provides methods for detecting target nucleic acids in a sample. Such methods may comprise contacting a sample with the first end of a lateral flow device as described herein. The first end of the lateral flow device may comprise a sample loading portion, wherein the sample flows from the sample loading portion of the substrate towards the first and second capture regions and generates a detectable signal.

A positive detectable signal may be any signal that can be detected using optical, fluorescent, chemiluminescent, electrochemical or other detection methods known in the art, as described elsewhere herein.

In one embodiment, the lateral flow device may be capable of detecting two different target nucleic acid sequences. In one embodiment, this detection of two different target nucleic acid sequences may occur simultaneously.

In one embodiment, the absence of target nucleic acid sequences in a sample elicits a detectable fluorescent signal at each capture region. In such instances, the absence of any target nucleic acid sequences in a sample may cause a detectable signal to appear at the first and second capture regions.

In one embodiment, the lateral flow device as described herein is capable of detecting three different target nucleic acid sequences. In specific embodiments, when the target nucleic acid sequences are absent from the sample, a fluorescent signal may be generated at each of the three capture regions. In such exemplary embodiments, a fluorescent signal may be absent at the capture region for the corresponding target nucleic acid sequence when the sample contains one or more target nucleic acid sequences.

Samples to be screened are loaded at the sample loading portion of the lateral flow substrate. The samples must be liquid samples or samples dissolved in an appropriate solvent, usually aqueous. The liquid sample reconstitutes the system reagents such that a detection reaction can occur. Intact reporter construct is bound at the first capture region by binding between the first binding agent and the first molecule. Likewise, the detection agent will begin to collect at the first binding region by binding to the second molecule on the intact reporter construct. If target molecule(s) are present in the sample, the TnpB protein collateral effect is activated. As activated TnpB protein comes into contact with the bound reporter construct, the reporter constructs are cleaved, releasing the second molecule to flow further down the lateral flow substrate towards the second binding region. The released second molecule is then captured at the second capture region by binding to the second binding agent, where additional detection agent may also accumulate by binding to the second molecule. Accordingly, if the target molecule(s) is not present in the sample, a detectable signal will appear at the first capture region, and if the target molecule(s) is present in the sample, a detectable signal will appear at the location of the second capture region.

In one embodiment, the invention provides a method for quantifying target nucleic acids in samples comprising distributing a sample or set of samples into one or more individual discrete volumes comprising two or more TnpB systems as described herein. The method may comprise using HDA to amplify one or more target molecules in the sample or set of samples, as described herein. The method may further comprise incubating the sample or set of samples under conditions sufficient to allow binding of the nucleic acid component molecules to one or more target molecules. The method may further comprise activating the TnpB protein via binding of the nucleic acid component molecules to the one or more target molecules. Activating the TnpB protein may result in modification of the detection construct such that a detectable positive signal is generated. The method may further comprise detecting the one or more detectable positive signals, wherein detection indicates the presence of one or more target molecules in the sample. The method may further comprise comparing the intensity of the one or more signals to a control to quantify the nucleic acid in the sample. The steps of amplifying, incubating, activating, and detecting may all be performed in the same individual discrete volume.

An “individual discrete volume” is a discrete volume or discrete space, such as a container, receptacle, or other defined volume or space that can be defined by properties that prevent and/or inhibit migration of nucleic acids and reagents necessary to carry out the methods disclosed herein, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof. By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the used of non-walled, or semipermeable is that some reagents, such as buffers, chemical activators, or other agents maybe passed in Applicants' through the discrete volume, while other material, such as target molecules, maybe maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain example embodiments, the individual discrete volumes are the wells of a microplate. In certain example embodiments, the microplate is a 96 well, a 384 well, or a 1536 well microplate.

Incubating the sample at either the amplification step or the extraction steps as described herein can be performed using heat sources known in the art. Advantageously, the heat source can be readily commercially available heating sources that do not require complicated instrumentation. Exemplary heating systems can include heating blocks, incubators, and/or water baths with temperatures maintained by commercially available sous-vide cookers. In this way, sample diagnostics can be performed without the requirement of expensive and proprietary equipment found primarily in diagnostic laboratory and hospital settings.

In certain example embodiments, paper-based microfluidics may be used for transfer of samples or reagents. For example, paper strips having wax barrier printed at a defined distance from the end of a paper dipstick may be used to define a volume of reagent or sample to be transferred. For example, a wax barrier may be printed across a paper dipstick to define a microliter volume such that when the dipstick is transferred into a volume of a reagent or sample only a microliter of said reagent or sample is absorbed onto the dipstick. The dipstick may be place in a second reagent mix, where the reagent or sample will diffuse into the reaction mixture. Such components allow for preparation and use of the assay without specialized equipment such as pipettors.

Optical means may be used to assess the presence and level of a given target molecule. In one embodiment, an optical sensor detects unmasking of a fluorescent masking agent. In one embodiment, the device of the present invention may include handheld portable devices for diagnostic reading of an assay (see e.g., Vashist et al., Commercial Smartphone-Based Devices and Smart Applications for Personalized Healthcare Monitoring and Management, Diagnostics 2014, 4(3), 104-128; mReader from Mobile Assay; and Holomic Rapid Diagnostic Test Reader).

As noted herein, certain embodiments allow detection via colorimetric change which has certain attendant benefits when embodiments are utilized in POC situations and or in resource poor environments where access to more complex detection equipment to readout the signal may be limited. However, portable embodiments disclosed herein may also be coupled with hand-held spectrophotometers that enable detection of signals outside the visible range. An example of a hand-held spectrophotometer device that may be used in combination with the present invention is described in Das et al. “Ultra-portable, wireless smartphone spectrophotometer for rapid, non-destructive testing of fruit ripeness.” Nature Scientific Reports. 2016, 6:32504, DOI: 10.1038/srep32504. Finally, In one embodiment utilizing quantum dot-based masking constructs, use of a hand-held UV light, or other suitable device, may be successfully used to detect a signal owing to the near complete quantum yield provided by quantum dots.

### Amplifying Target Molecules

The step of amplifying one or more target molecules can comprise amplification systems known in the art. In one embodiment, amplification is isothermal. In certain example embodiments, target RNAs and/or DNAs may be amplified prior to activating the TnpB protein. Any suitable RNA or DNA amplification technique may be used. In certain embodiments, the amplifying step may take less than about 1 hour, 50 minutes, 40 minutes, 30 minutes, 25 minutes, 20 minutes or 15 minutes, which may depend on the sample, starting concentrations and nature of amplification used.

In one embodiment, the amplifying of the target molecules and the detection of the target molecules can be performed in a single reaction, for example, a ‘one-pot’ method. General guidance for use of a single-pot approach can be as described in Gootenberg, et al., Science 2018 Apr. 27: 360(6387) 439-444 (using Cas13, Cas12a and Csm6 generally, detecting multiple targets in a single reaction, and specifically performing DNA extraction in a sample and using as input for direct detection at Figure S33); and Ding et al., “All-in-One Dual CRISPR-Cas12a (AIOD-CRISPR) Assay: A Case for Rapid, Ultrasensitive and Visual Detection of Novel Coronavirus SARS-COV-2 and HIV Virus,” doi: 10.1101/2020.03.19.998724, biorxiv preprint (utilizing a pair of crRNAs with dual CRISPR-Cas12a detection for a one-pot approach to target-specific nucleic acid detection).

In certain example embodiments, the RNA or DNA amplification is an isothermal amplification. In certain example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR, multiple displacement amplification (MDA), rolling circle amplification (RCA), ligase chain reaction (LCR), or ramification amplification method (RAM).

The amplifying of target molecules can be optimized by methods as detailed herein. In an aspect, the design optimizes the primers used in the amplification. In particular aspects, the isothermal amplification is used with TnpB systems. In either approach, design considerations can follow a rational design for optimization of the reactions. Optimization of the methods as disclosed herein can include first screening primers to identify one or more sets of primers that work well for a particular target, TnpB protein and/or reaction. Once the primers have been screened, titration of magnesium concentration can be performed to identify an optimal magnesium concentration for higher signal to noise readout. In an example, varying additives with specific primers, target, TnpB protein, temperature, and other additive concentrations within the reaction can be identified. Optimization can be made with the goal of reducing the number of steps and buffer exchanges that have to occur in the reaction, simplifying the reaction and reducing the risks of contamination at transfer steps. Similarly, optimizing the salt levels as well as the type of salt utilized can further facilitate and optimize the one-pot detections disclosed herein.

### Loop-Mediated Isothermal Amplification

In certain example embodiments, a loop-mediated isothermal amplification (LAMP) reaction may be used to target nucleic acids, which encompasses both LAMP and RT-LAMP reactions. LAMP can be performed with a four-primer system for isothermal nucleic acid amplification in conjunction with a polymerase. Notomi et al., Nucleic Acids Res. 2000, 28, 12, Nagamine et al., Molecular and Cellular Probes (2002) 16, 223-229, doi: 10.1006/mcpr.2002.0415. When performing LAMP with a 4-primer system, two loop-forming inner primers, denoted as FIP and BIP, are provided with two outer primers, F3 and B3. The inner primers each contain two distinct sequences, one for priming in the first stage of the amplification and the other sequence for self-priming in subsequent amplification states. The two outer primers initiate strand displacement of nucleic acid strands initiated from the FIP and BIP primers, thereby generating formation of loops and strand displacement nucleic acid synthesis utilizing the provided polymerase. LAMP can be conducted with two to six primers, ranging from only the two loop-forming primers, up to at least the addition of 2 additional primers, LF and LB along with the two outer primers and two inner primers. LAMP technologies advantageously have high specificity and can work at a variety of pH and temperature. In a preferred aspect, the LAMP is an isothermal reaction at between about 45° C. to 75° C., 55 to 70° C. or 60° C. to 65° C. Colorimetric LAMP (Y. Zhang et al., doi: 10.1101/2020.92.26.20028373), RT-LAMP (Lamb et al., doi: 10.1101/2020.02.19.20025155; and Yang et al., doi: 10.1101/2020.03.02.20030130) have been developed for detection of COVID-19, and are incorporated herein by reference in their entirety.

In one embodiment, the LAMP reagents may include Bst 2.0+RTx or Bst 3.0 from New England Biolabs. In one embodiment, the LAMP reagents may comprise colorimetric or fluorescent detection. Detection of LAMP products can be accomplished using colorimetric tools, such as hydroxy napthol blue (see, e.g., Goto, M., et al., Colorimetric detection of loop-mediated isothermal amplification reaction by using hydroxy naphthol blue. Biotechniques, 2009. 46(3): p. 167-72.) leuco triphenylmethane dyes (see, e.g., Miyamoto, S., et al., Method for colorimetric detection of double-stranded nucleic acid using leuco triphenylmethane dyes. Anal Biochem, 2015. 473: p. 28-33) and pH-snesitive dyes (see, e.g., Tanner, N. A., Y. Zhang, and T. C. Evans, Jr., Visual detection of isothermal nucleic acid amplification using pH-sensitive dyes. Biotechniques, 2015. 58(2): p. 59-68); as well as fluorescent detection (see, e.g., Yu et al., Clinical Chemistry, hvaa102, doi: 10.1093/clinchem/hvaa102 12 May 2020), including use of quenching probes (see, e.g., Shirato et al., J Virol Methods. 2018 August; 258:41-48. doi: 10.1016/j.jviromet.2018.05.006).

In an aspect, the primer sets for LAMP are designed to amplify one or more target sequences, generating amplicons that comprise the one or more target sequences. Optionally, the primers can comprise barcodes that can be designed as described elsewhere herein. Incubating to a temperature sufficient for LAMP amplification, e.g., 50° C.-72° C., more preferably 55° C. to 65° C., using a polymerase and, optionally a reverse transcriptase (in the event RT-LAMP is utilized). Preferably the enzymes utilized in the LAMP reaction are heat-stabilized. LAMP primer sites have been designed, see, e.g., Park et al., “Development of Reverse Transcription Loop-Mediated Isothermal Amplification Assays Targeting SARS-COV-2” J. of Mol. Diag. (2020). Optionally, a control template is further provided with the sample, which may differ from the target sequence but share primer binding sites. In an exemplary embodiment, visual read out of the detection results can be accomplished using commercially-available lateral flow substrate, e.g., a commercially available paper substrate.

### NASBA

In certain example embodiments, the RNA or DNA amplification is NASBA, which is initiated with reverse transcription of target RNA by a sequence-specific reverse primer to create a RNA/DNA duplex. RNase H is then used to degrade the RNA template, allowing a forward primer containing a promoter, such as the T7 promoter, to bind and initiate elongation of the complementary strand, generating a double-stranded DNA product. The RNA polymerase promoter-mediated transcription of the DNA template then creates copies of the target RNA sequence. Importantly, each of the new target RNAs can be detected by the nucleic acid component molecules thus further enhancing the sensitivity of the assay. Binding of the target RNAs by the nucleic acid component molecules then leads to activation of the TnpB protein and the methods proceed as outlined above. The NASBA reaction has the additional advantage of being able to proceed under moderate isothermal conditions, for example at approximately 41° C., making it suitable for systems and devices deployed for early and direct detection in the field and far from clinical laboratories.

### RPA

In certain other example embodiments, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, an RNA polymerase promoter, such as a T7 promoter, is added to one of the primers. This results in an amplified double-stranded DNA product comprising the target sequence and an RNA polymerase promoter. After, or during, the RPA reaction, an RNA polymerase is added that will produce RNA from the double-stranded DNA templates. The amplified target RNA can then in turn be detected by the TnpB system. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above.

### Transposase Based Amplification

Embodiments disclosed herein provide systems and methods for isothermal amplification of target nucleic acid sequences by contacting oligonucleotides containing the target nucleic acid sequence with a transposon complex. The oligonucleotides may be single stranded or double stranded RNA, DNA, or RNA/DNA hybrid oligonucleotides. The transposon complex comprises a transposase and a transposon sequence comprising one or more RNA polymerase promoters. The transposase facilitates insertion of the one or more RNA polymerase promoters into the oligonucleotide. An RNA polymerase promoter can then transcribe the target nucleic acid sequence from the inserted one or more RNA polymerase promoters. One advantage of this system is that there is no need to heat or melt double-stranded DNA templates, since RNA polymerase polymerases require a double-stranded template. Such isothermal amplification is fast and simple, obviating the need for complicated and expensive instrumentation for denaturation and cooling. In certain example embodiment the RNA polymerase promoter is a native of modified T7 RNA promoter.

The term “transposon”, as used herein, refers to a nucleic acid segment, which is recognized by a transposase or an integrase enzyme and which is an essential component of a functional nucleic acid-protein complex (i.e., a transposome) capable of transposition. The term “transposase” as used herein refers to an enzyme, which is a component of a functional nucleic acid-protein complex capable of transposition and which is mediating transposition. The term “transposase” also refers to integrases from retrotransposons or of retroviral origin. Transposon complexes form between a transposase enzyme and a fragment of double stranded DNA that contains a specific binding sequence for the enzyme, termed “transposon end”. The sequence of the transposon binding site can be modified with other bases, at certain positions, without affecting the ability for transposon complex to form a stable structure that can efficiently transpose into target DNA.

In embodiments provided herein, the transposon complex may comprise a transposase and a transposon sequence comprising one or more RNA polymerase promoters. The term “promoter” refers to a region of DNA involved in binding the RNA polymerase to initiate transcription. In specific embodiments, the RNA polymerase promoter may be a T7 RNA polymerase promoter. The T7 RNA promoter may be inserted into the double-stranded polynucleotide using the transposase. In one embodiment, insertion of the T7 RNA polymerase promoter into the oligonucleotide may be random.

The frequency of transposition is very low for most transposons, which use complex mechanisms to limit activity. Tn5 transposase, for example, utilizes a DNA binding sequence that is suboptimal and the C-terminus of the transposase interferes with DNA binding. Mechanisms involved in Tn5 transposition have been carefully characterized by Reznikoff and colleagues. Tn5 transposes by a cut-and-paste mechanism. The transposon has two pairs of 19 bp elements that are utilized by the transposase: outside elements (OE) and inside elements (IE). One transposase monomer binds to each of the two elements that are utilized. After a monomer is bound to each end of the transposon, the two monomers dimerize, forming a synapse. Vectors with donor backbones of at least 200 bp, but less than 1000 bp, are most functional for transposition in bacteria. Transposon cleavage occurs by trans catalysis and only when monomers bound to each DNA end are in a synaptic complex. Tn5 transposes with a relaxed target site selection and can therefore insert into target DNA with little to no target sequence specificity.

The natural downregulation of Tn5 transposition can be overcome by selection of a hyperactive transposase and by optimizing the transposase-binding elements [York et al. 1998]. A mosaic element (ME), made by modification of three bases of the wild type OE, led to a 50-fold increase in transposition events in bacteria as well as cell-free systems. The combined effect of the optimized ME and hyperactive mutant transposase is estimated to result in a 100-fold increase in transposition activity. Goryshin et al showed that preformed Tn5 transposition complexes could be functionally introduced into bacterial or yeast by electroporation [Goryshin et al. 2000]. Linearization of the DNA, to have inverted repeats precisely positioned at both ends of the transposon, allowed Goryshin and coworkers to bypass the cutting step of transposition thus enhancing transposition efficiency.

In one embodiment, the transposase may be used to tagment the oligonucleotide sequence comprising the target sequence. The term “tagmentation” refers to a step in the Assay for Transposase Accessible Chromatin using sequencing (ATAC-seq) as described. (See, Buenrostro, J. D., Giresi, P. G., Zaba, L. C., Chang, H. Y., Greenleaf, W. J., Transposition of native chromatin for fast and sensitive epigenomic profiling of open chromatin, DNA-binding proteins and nucleosome position. Nature methods 2013; 10 (12): 1213-1218). Specifically, a hyperactive Tn5 transposase loaded in vitro with adapters for high-throughput DNA sequencing, can simultaneously fragment and tag a genome with sequencing adapters. In one embodiment the adapters are compatible with the methods described herein.

In one embodiment, the transposase may be a Tn5 transposase. In one embodiment, the transposase may be a variant of a Tn5 transposase, or an engineered transposase. Transposases may be engineered using any method known in the art. The engineered transposase may be optimized to function at a temperature ranging from 30° C. to 45° C., 35° C. to 40° C. or any temperature in between. The engineered transposase may be optimized to release from the oligonucleotide at a faster rate compared to a wild type transposase.

In one embodiment, the transposase may be a Tn5 transposase, a Mu transposase, or a Tn7 transposase. Transposition efficiency in vitro may vary depending on the transposon system used. Generally, Tn5 and Mu transposases effect higher levels of transposition efficiency. In one embodiment, insertion may be random. In one embodiment, insertion may occur in GC rich regions of the target sequence.

In one embodiment, the transposon sequence may comprise two 19 base pair Mosaic End (ME) Tn5 transposase recognition sequences. Tn5 transposases will generally transpose any DNA sequence contained between such short 19 base pair ME Tn5 transposase recognition sequences.

In one embodiment, use of a transposase allows for separation of a double-stranded polynucleotide in the absence of heat or melting. Approaches can be adapted from those described in PCT/US2019/039195, incorporated herein by reference.

### Nickase Dependent Amplification

In an embodiment of the invention may comprise nickase-based amplification. The nicking enzyme may be a TnpB protein. Accordingly, the introduction of nicks into dsDNA can be programmable and sequence-specific. In an embodiment of the invention, two guides can be designed to target opposite strands of a dsDNA target. According to the invention, the nickase can be TnpB, or one may use any CRISPR protein such as Cpf1, C2c1, Cas9, or any ortholog or CRISPR protein that cleaves or is engineered to cleave a single strand of a DNA duplex. In a particular embodiment, the TnpB is utilized in the nickase dependent amplification. The nicked strands may then be extended by a polymerase. In an embodiment, the locations of the nicks are selected such that extension of the strands by a polymerase is towards the central portion of the target duplex DNA between the nick sites. In one embodiment, primers are included in the reaction capable of hybridizing to the extended strands followed by further polymerase extension of the primers to regenerate two dsDNA pieces: a first dsDNA that includes the first strand TnpB guide site or both the first and second strand TnpB guide sites, and a second dsDNA that includes the second strand TnpB guide site or both the first and second strand TnpB guide sites. These pieces continue to be nicked and extended in a cyclic reaction that exponentially amplifies the region of the target between nicking sites. Alternatively, a CRISPR-Cas protein instead of TnpB can be used for nickase-based amplification, and such methods are known in the art.

The amplification can be isothermal and selected for temperature. In one embodiment, the amplification proceeds rapidly at 37 degrees. In other embodiments, the temperature of the isothermal amplification may be chosen by selecting a polymerase (e.g., Bsu, Bst, Phi29, klenow fragment etc.) operable at a different temperature.

Thus, whereas nicking isothermal amplification techniques use nicking enyzmes with fixed sequence preference (e.g., in nicking enzyme amplification reaction or NEAR), which requires denaturing of the original dsDNA target to allow annealing and extension of primers that add the nicking substrate to the ends of the target, use of a reprogrammable nickase wherein the nicking sites can be programed via RNA molecules means that no denaturing step is necessary, enabling the entire reaction to be truly isothermal. This also simplifies the reaction because these primers that add the nicking substrate are different than the primers that are used later in the reaction, meaning that NEAR requires two primer sets (i.e., 4 primers) while Cpf1 nicking amplification only requires one primer set (i.e., two primers). This makes nicking Cpf1 amplification much simpler and easier to operate without complicated instrumentation to perform the denaturation and then cooling to the isothermal temperature.

In an aspect, the isothermal amplification reagents may be utilized with a thermostable TnpB protein. The combination of thermostable protein and isothermal amplification reagents may be utilized to further improve reaction times for detection and diagnostics.

Accordingly, in certain example embodiments the systems disclosed herein may include amplification reagents. Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present invention.

A salt, such as magnesium chloride (MgCl2), potassium chloride (KCl), or sodium chloride (NaCl), may be included in an amplification reaction, such as PCR, in order to improve the amplification of nucleic acid fragments. Although the salt concentration will depend on the particular reaction and application, in one embodiment, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present invention and as described herein. In certain preferred embodiments, when polynucleotide extraction beads such as magnetic beads are utilized, a Plant QuickExtract solution can be used in combination with a KCl buffer in optimized detection methods according to the present disclosure.

Other components of a biological or chemical reaction may include a cell lysis component in order to break open or lyse a cell for analysis of the materials therein. A cell lysis component may include, but is not limited to, a detergent, a salt as described above, such as NaCl, KCl, ammonium sulfate [(NH4)2SO4], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application, and may be specific to the reaction in some cases. Amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the invention may be any specific or general polymerase known in the art and useful or the invention, including Taq polymerase, Q5 polymerase, or the like.

In one embodiment, amplification reagents as described herein may be appropriate for use in hot-start amplification. Hot start amplification may be beneficial in one embodiment to reduce or eliminate dimerization of adaptor molecules or oligos, or to otherwise prevent unwanted amplification products or artifacts and obtain optimum amplification of the desired product. Many components described herein for use in amplification may also be used in hot-start amplification. In one embodiment, reagents or components appropriate for use with hot-start amplification may be used in place of one or more of the composition components as appropriate. For example, a polymerase or other reagent may be used that exhibits a desired activity at a particular temperature or other reaction condition. In one embodiment, reagents may be used that are designed or optimized for use in hot-start amplification, for example, a polymerase may be activated after transposition or after reaching a particular temperature. Such polymerases may be antibody-based or aptamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents.

Amplification of nucleic acids may be performed using specific thermal cycle machinery or equipment, and may be performed in single reactions or in bulk, such that any desired number of reactions may be performed simultaneously. In one embodiment, amplification may be performed using microfluidic or robotic devices, or may be performed using manual alteration in temperatures to achieve the desired amplification. In one embodiment, optimization may be performed to obtain the optimum reactions conditions for the particular application or materials. One of skill in the art will understand and be able to optimize reaction conditions to obtain sufficient amplification.

In one embodiment, detection of DNA with the methods or systems of the invention requires transcription of the (amplified) DNA into RNA prior to detection.

It will be evident that detection methods of the invention can involve nucleic acid amplification and detection procedures in various combinations. The nucleic acid to be detected can be any naturally occurring or synthetic nucleic acid, including but not limited to DNA and RNA, which may be amplified by any suitable method to provide an intermediate product that can be detected. Detection of the intermediate product can be by any suitable method including but not limited to binding and activation of a TnpB protein which produces a detectable signal moiety by direct or collateral activity.

### Helicase-Dependent Amplification

In helicase-dependent amplification, a helicase enzyme is used to unwind a double stranded nucleic acid to generate templates for primer hybridization and subsequent primer-extension. This process utilizes two oligonucleotide primers, each hybridizing to the 3′-end of either the sense strand containing the target sequence or the anti-sense strand containing the reverse-complementary target sequence. The HDA reaction is a general method for helicase-dependent nucleic acid amplification.

In combining this method with a TnpB detection system, the target nucleic acid may be amplified by opening R-loops of the target nucleic acid using first and second TnpB complexes. The first and second strand of the target nucleic acid may thus be unwound using a helicase, allowing primers and polymerase to bind and extend the DNA under isothermal conditions.

The term “helicase” refers here to any enzyme capable of unwinding a double stranded nucleic acid enzymatically. For example, helicases are enzymes that are found in all organisms and in all processes that involve nucleic acid such as replication, recombination, repair, transcription, translation and RNA splicing. (Kornberg and Baker, DNA Replication, W. H. Freeman and Company (2^{nd} ed. (1992)), especially chapter 11). Any helicase that translocates along DNA or RNA in a 5′ to 3′ direction or in the opposite 3′ to 5′ direction may be used in present embodiments of the invention. This includes helicases obtained from prokaryotes, viruses, archaea, and eukaryotes or recombinant forms of naturally occurring enzymes as well as analogues or derivatives having the specified activity. Examples of naturally occurring DNA helicases, described by Kornberg and Baker in chapter 11 of their book, DNA Replication, W. H. Freeman and Company (2^{nd} ed. (1992)), include E. coli helicase I, II, III, & IV, Rep, DnaB, PriA, PcrA, T4 Gp41helicase, T4 Dda helicase, T7 Gp4 helicases, SV40 Large T antigen, yeast RAD. Additional helicases that may be useful in HDA include RecQ helicase (Harmon and Kowalczykowski, J. Biol. Chem. 276:232-243 (2001)), thermostable UvrD helicases from T. tengcongensis (disclosed in this invention, Example XII) and T. thermophilus (Collins and McCarthy, Extremophiles. 7:35-41. (2003)), thermostable DnaB helicase from T. aquaticus (Kaplan and Steitz, J. Biol. Chem. 274:6889-6897 (1999)), and MCM helicase from archaeal and eukaryotic organisms (Grainge et al., Nucleic Acids Res. 31:4888-4898(2003)).

A traditional definition of a helicase is an enzyme that catalyzes the reaction of separating/unzipping/unwinding the helical structure of nucleic acid duplexes (DNA, RNA or hybrids) into single-stranded components, using nucleoside triphosphate (NTP) hydrolysis as the energy source (such as ATP). However, it should be noted that not all helicases fit this definition anymore. A more general definition is that they are motor proteins that move along the single-stranded or double stranded nucleic acids (usually in a certain direction, 3′ to 5′ or 5 to 3, or both), i.e., translocases, that can or cannot unwind the duplexed nucleic acid encountered. In addition, some helicases simply bind and “melt” the duplexed nucleic acid structure without an apparent translocase activity.

Helicases exist in all living organisms and function in all aspects of nucleic acid metabolism. Helicases are classified based on the amino acid sequences, directionality, oligomerization state and nucleic-acid type and structure preferences. The most common classification method was developed based on the presence of certain amino acid sequences, called motifs. According to this classification helicases are divided into 6 super families: SF1, SF2, SF3, SF4, SF5 and SF6. SF1 and SF2 helicases do not form a ring structure around the nucleic acid, whereas SF3 to SF6 do. Superfamily classification is not dependent on the classical taxonomy.

DNA helicases are responsible for catalyzing the unwinding of double-stranded DNA (dsDNA) molecules to their respective single-stranded nucleic acid (ssDNA) forms. Although structural and biochemical studies have shown how various helicases can translocate on ssDNA directionally, consuming one ATP per nucleotide, the mechanism of nucleic acid unwinding and how the unwinding activity is regulated remains unclear and controversial (T. M. Lohman, E. J. Tomko, C. G. Wu, “Non-hexameric DNA helicases and translocases: mechanisms and regulation,” Nat Rev Mol Cell Biol 9:391-401 (2008)). Since helicases can potentially unwind all nucleic acids encountered, understanding how their unwinding activities are regulated can lead to harnessing helicase functions for biotechnology applications.

The term “HDA” refers to Helicase Dependent Amplification, which is an in vitro method for amplifying nucleic acids by using a helicase preparation for unwinding a double stranded nucleic acid to generate templates for primer hybridization and subsequent primer-extension. This process utilizes two oligonucleotide primers, each hybridizing to the 3′-end of either the sense strand containing the target sequence or the anti-sense strand containing the reverse-complementary target sequence. The HDA reaction is a general method for helicase-dependent nucleic acid amplification.

The invention comprises use of any suitable helicase known in the art. These include, but are not necessarily limited to, UvrD helicase, CRISPR-Cas3 helicase, E. coli helicase I, E. coli helicase II, E. coli helicase III, E. coli helicase IV, Rep helicase, DnaB helicase, PriA helicase, PcrA helicase, T4 Gp41 helicase, T4 Dda helicase, SV40 Large T antigen, yeast RAD helicase, RecD helicase, RecQ helicase, thermostable T. tengcongensis UvrD helicase, thermostable T. thermophilus UvrD helicase, thermostable T. aquaticus DnaB helicase, Dda helicase, papilloma virus El helicase, archaeal MCM helicase, eukaryotic MCM helicase, and T7 Gp4 helicase.

In particularly preferred embodiments, the helicase comprises a super mutation. In particular embodiments, although the E coli mutation has been described, the mutations were generated by sequence alignment (e.g., D409A/D410A for TteUvrd) and result in thermophilic enzymes working at lower temperatures like 37° C., which is advantageous for amplification methods and systems described herein. In one embodiment, the super mutations is an aspartate to alanine mutation, with position based on sequence alignment. In one embodiment, the super mutant helicase is selected from WP_003870487.1 Thermoanaerobacter ethanolicus 403/404, WP_049660019.1 Bacillus sp. FJAT-27231 407/408, WP_034654680.1 Bacillus megaterium 415/416, WP_095390358.1 Bacillus simplex 407/408, and WP_055343022.1 Paeniclostridium sordellii 402/403.

### Incubating

Methods of detection and/or extraction using the systems disclosed herein can comprise incubating the sample or set of samples under conditions sufficient to allow binding of the nucleic acid component molecules to one or more target molecules. Exstraction can comprise incubating the sample under conditions sufficient to allow release of viral RNA present in the sample, which may comprise incubating at 22° C. to 60° C. for 30 to 70 minutes or at 90° C.-100° C. for about 10 minutes.

In certain example embodiments, the incubation time of the amplifying and detecting in the present invention may be shortened. The assay may be performed in a period of time required for an enzymatic reaction to occur. One skilled in the art can perform biochemical reactions in 5 minutes (e.g., 5 minute ligation). Incubating may occur at one or more temperatures over timeframes between about 10 minutes and 90 minutes, preferably less than 90 minutes, 75 minutes, 60 minutes, 45 minutes, 30 minutes, 25 minutes, 20 minutes, 15 minutes, or 10 minutes depending on sample, reagents and components of the system. In one embodiment, incubating for the amplification is performed at one or more temperatures between about 20° C. and 80° C., In one embodiment, about 37° C. In one embodiment, incubating for the amplification is performed at one or more temperatures between about 55° C. and 65° C., between about 59° C. and 61° C., In one embodiment, about 60° C.

### Activating

In certain example embodiment, activating of the TnpB protein occurs via binding of the TnpB complex via the nucleic acid component molecule to the one or more target molecules, wherein activating the TnpB protein results in modification of the detection construct such that a detectable signal is generated.

### Detecting a Signal

Detecting may comprise visual observance of a positive signal relative to a control. Detecting may comprise a loss of signal or presence of signal at one or more capture regions, for example colorimetric detection, or fluorescent detection. In certain example embodiments, further modifications may be introduced that further amplify the detectable positive signal. For example, activated TnpB protein collateral activation may be used to generate a secondary target or additional nucleic acid component molecule sequence, or both. In one example embodiment, the reaction solution would contain a secondary target that is spiked in at high concentration. The secondary target may be distinct from the primary target (i.e., the target for which the assay is designed to detect) and in certain instances may be common across all reaction volumes. A secondary nucleic acid component molecule sequence for the secondary target may be protected, e.g., by a secondary structural feature such as a hairpin with an RNA loop, and unable to bind the second target or the TnpB protein. Cleavage of the protecting group by an activated TnpB r protein (i.e., after activation by formation of complex with the primary target(s) in solution) and formation of a complex with free TnpB protein in solution and activation from the spiked in secondary target. In certain other example embodiments, a similar concept is used with free nucleic acid component molecule sequence to a secondary target and protected secondary target. Cleavage of a protecting group off the secondary target would allow additional TnpB protein, nucleic acid component sequence, secondary target sequence to form. In yet another example embodiment, activation of TnpB protein by the primary target(s) may be used to cleave a protected or circularized primer, which would then be released to perform an isothermal amplification reaction, such as those disclosed herein, on a template for either secondary nucleic acid component sequence, secondary target, or both. Subsequent transcription of this amplified template would produce more secondary nucleic acid component molecule sequence and/or secondary target sequence, followed by additional TnpB protein collateral activation.

### Quantifying

In particular methods, comparing the intensity of the one or more signals to a control is performed to quantify the nucleic acid in the sample. The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control patient (can be stored sample or previous sample measurement) with a known outcome; normal tissue, fluid, or cells isolated from a subject, such as a normal patient or the patient having a condition of interest.

The intensity of a signal is “significantly” higher or lower than the normal intensity if the signal is greater or less, respectively, than the normal or control level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternatively, the signal can be considered “significantly” higher or lower than the normal and/or control signal if the amount is at least about two, and preferably at least about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, two times, three times, four times, five times, or more, or any range in between, such as 5%-100%, higher or lower, respectively, than the normal and/or control signal. Such significant modulation values can be applied to any metric described herein, such as altered level of expression, altered activity, changes in biomarker inhibition, changes in test agent binding, and the like.

In one embodiment, the detectable positive signal may be a loss of fluorescent signal or colorimetric relative to a control, as described herein. In one embodiment, the detectable positive signal may be detected on a lateral flow device, as described herein.

### Applications of Detection Methods

Systems and methods can be designed for the detection and diagnosis of microbes, including bacterial, fungi and viral microbes. In an aspect, the systems may comprise multiplex detection of multiple variants of viral infections, including coronavirus, different viruses which may be related coronaviruses or respiratory viruses, or a combination thereof. In embodiments, assays can be performed for a variety of viruses and viral infections, including acute respiratory infections using the disclosure detailed herein. The systems can comprise two or more TnpB systems to multiplex, as described elsewhere herein, to detect a plurality of respiratory infections or viral infections, including coronavirus. The coronavirus is a positive-sense single stranded RNA family of viruses, infecting a variety of animals and humans. SARS-COV is one type of coronavirus infection, as well as MERS-COV Detection of one or more coronaviruses are envisioned, including the 2019-nCOV detected in Wuhan City. Sequences of the 2019-nCOV are available at GISAID accession no. EPI_ISL_402124 and EPI_ISL_402127-402130, and described in DOI: 10.1101/2020.01.22.914952. Further deposits of the SARS-COV-2 deposited in the GISAID platform include EP_ISL_402119-402121 and EP_ISL_402123-402124; see also GenBank Accession No. MN908947.3.

Target molecule detection can comprise two or more detection systems utilizing TnpB proteins. The TnpB protein may preferably be thermostable, with multiplexing designed such that different TnpB proteins with different sequence specificities, operable temperatures, or cutting preferences can be used.

A multiplex embodiment can be designed to track one or more variants of coronavirus or one or more variants of coronavirus, including SARS-COV-2, in combination with other viruses, for example, Human respiratory syncytial virus, Middle East respiratory syndrome (MERS) coronavirus, Severe acute respiratory syndrome-related (SARS) coronavirus, and influenza. In embodiments, assays can be done in multiplex to detect multiple variants of coronavirus, different viruses which may be related coronaviruses or respiratory viruses, or a combination thereof. In an aspect, each assay can take place in an individual discrete volume. An “individual discrete volume” is a discrete volume or discrete space, such as a container, receptacle, or other defined volume or space that can be defined by properties that prevent and/or inhibit migration of nucleic acids and reagents necessary to carry out the methods disclosed herein, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof. By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the use of non-walled, or semipermeable is that some reagents, such as buffers, chemical activators, or other agents maybe passed in Applicants' through the discrete volume, while other material, such as target molecules, maybe maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain example embodiments, the individual discrete volumes are the wells of a microplate. In certain example embodiments, the microplate is a 96 well, a 384 well, or a 1536 well microplate.

In certain example embodiments, the systems, devices, and methods, disclosed herein are directed to detecting the presence of one or more microbial agents in a sample, such as a biological sample obtained from a subject. In certain example embodiments, the microbe may be a bacterium, a fungus, a yeast, a protozoan, a parasite, or a virus. Accordingly, the methods disclosed herein can be adapted for use in other methods (or in combination) with other methods that require quick identification of microbe species, monitoring the presence of microbial proteins (antigens), antibodies, antibody genes, detection of certain phenotypes (e.g., bacterial resistance), monitoring of disease progression and/or outbreak, and antibiotic screening. Because of the rapid and sensitive diagnostic capabilities of the embodiments disclosed here, detection of microbe species type, down to a single nucleotide difference, and the ability to be deployed as a POC device, the embodiments disclosed herein may be used as guide therapeutic regimens, such as a selection of the appropriate antibiotic or antiviral. The embodiments disclosed herein may also be used to screen environmental samples (air, water, surfaces, food etc.) for the presence of microbial contamination.

Disclosed is a method to identify microbial species, such as bacterial, viral, fungal, yeast, or parasitic species, or the like. Particular embodiments disclosed herein describe methods and systems that will identify and distinguish microbial species within a single sample, or across multiple samples, allowing for recognition of many different microbes. The present methods allow the detection of pathogens and distinguishing between two or more species of one or more organisms, e.g., bacteria, viruses, yeast, protozoa, and fungi or a combination thereof, in a biological or environmental sample, by detecting the presence of a target nucleic acid sequence in the sample. A positive signal obtained from the sample indicates the presence of the microbe. Multiple microbes can be identified simultaneously using the methods and systems of the invention, by employing the use of more than one effector protein, wherein each effector protein targets a specific microbial target sequence. In this way, a multi-level analysis can be performed for a particular subject in which any number of microbes can be detected at once, for example, a subject with unknown respiratory infection, having symptoms of coronavirus, or an individual at risk or having been exposed to coronavirus. In one embodiment, simultaneous detection of multiple microbes may be performed using a set of probes that can identify one or more microbial species.

### Microbe Detection

In one embodiment, a method for detecting microbes in samples is provided comprising distributing a sample or set of samples into one or more individual discrete volumes, the individual discrete volumes comprising a TNPB system as described herein; incubating the sample or set of samples under conditions sufficient to allow binding of the one or more nucleic acid component molecules to one or more microbe-specific targets; activating the TnpB protein via binding of the one or more nucleic acid component molecules to the one or more target molecules, wherein activating the TnpB protein results in modification of the RNA-based masking construct such that a detectable positive signal is generated; and detecting the detectable positive signal, wherein detection of the detectable positive signal indicates a presence of one or more target molecules in the sample. The one or more target molecules may be mRNA, gDNA (coding or non-coding), trRNA, or rRNA comprising a target nucleotide tide sequence that may be used to distinguish two or more microbial species/strains from one another. The nucleic acid component molecules may be designed to detect target sequences. The embodiments disclosed herein may also utilize certain steps to improve hybridization between nucleic acid component molecule and target RNA sequences. Methods for enhancing ribonucleic acid hybridization are disclosed in WO 2015/085194, entitled “Enhanced Methods of Ribonucleic Acid Hybridization” which is incorporated herein by reference. The microbe-specific target may be RNA or DNA or a protein. If DNA method may further comprise the use of DNA primers that introduce a RNA polymerase promoter as described herein. If the target is a protein, then the method will utilize aptamers and steps specific to protein detection described herein.

### Detection of Single Nucleotide Variants

In one embodiment, one or more identified target sequences may be detected using nucleic acid component molecules that are specific for and bind to the target sequence as described herein. The systems and methods of the present invention can distinguish even between single nucleotide polymorphisms present among different microbial species and therefore, use of multiple nucleic acid component molecules in accordance with the invention may further expand on or improve the number of target sequences that may be used to distinguish between species. For example, in one embodiment, the one or more nucleic acid component molecules may distinguish between microbes at the species, genus, family, order, class, phylum, kingdom, or phenotype, or a combination thereof.

Detection Based on rRNA Sequences

In certain example embodiments, the devices, systems, and methods disclosed herein may be used to distinguish multiple microbial species in a sample. In certain example embodiments, identification may be based on ribosomal RNA sequences, including the 16S, 23S, and 5S subunits. Methods for identifying relevant rRNA sequences are disclosed in U.S. Patent Application Publication No. 2017/0029872. In certain example embodiments, a set of nucleic acid component molecule may be designed to distinguish each species by a variable region that is unique to each species or strain. nucleic acid component molecules may also be designed to target RNA genes that distinguish microbes at the genus, family, order, class, phylum, kingdom levels, or a combination thereof. In certain example embodiments where amplification is used, a set of amplification primers may be designed to flanking constant regions of the ribosomal RNA sequence and a nucleic acid component molecule designed to distinguish each species by a variable internal region. In certain example embodiments, the primers and nucleic acid component molecules may be designed to conserved and variable regions in the 16S subunit respectfully. Other genes or genomic regions that uniquely variable across species or a subset of species such as the RecA gene family, RNA polymerase β subunit, may be used as well. Other suitable phylogenetic markers, and methods for identifying the same, are discussed for example in Wu et al. arXiv:1307.8690 [q-bio.GN].

In certain example embodiments, a method or diagnostic is designed to screen microbes across multiple phylogenetic and/or phenotypic levels at the same time. For example, the method or diagnostic may comprise the use of multiple TnpB systems with different nucleic acid component molecules. A first set of nucleic acid component molecules may distinguish, for example, between mycobacteria, gram positive, and gram negative bacteria. These general classes can be even further subdivided. For example, nucleic acid componentss could be designed and used in the method or diagnostic that distinguish enteric and non-enteric within gram negative bacteria. A second set of nucleic acid component molecules can be designed to distinguish microbes at the genus or species level. Thus, a matrix may be produced identifying all mycobacteria, gram positive, gram negative (further divided into enteric and non-enteric) with each genus of species of bacteria identified in a given sample that fall within one of those classes. The foregoing is for example purposes only. Other means for classifying other microbe types are also contemplated and would follow the general structure described above.

### Screening for Drug Resistance

In certain example embodiments, the devices, systems and methods disclosed herein may be used to screen for microbial genes of interest, for example antibiotic and/or antiviral resistance genes. nucleic acid component molecules may be designed to distinguish between known genes of interest. Samples, including clinical samples, may then be screened using the embodiments disclosed herein for detection of such genes. The ability to screen for drug resistance at POC would have tremendous benefit in selecting an appropriate treatment regime. In certain example embodiments, the antibiotic resistance genes are carbapenemases including KPC, NDM1, CTX-M15, OXA-48. Other antibiotic resistance genes are known and may be found for example in the Comprehensive Antibiotic Resistance Database (Jia et al. “CARD 2017: expansion and model-centric curation of the Comprehensive Antibiotic Resistance Database.” Nucleic Acids Research, 45, D566-573).

Ribavirin is an effective antiviral that hits a number of RNA viruses. Several clinically important viruses have evolved ribavirin resistance including Foot and Mouth Disease Virus doi: 10.1128/JVI.03594-13; polio virus (Pfeifer and Kirkegaard. PNAS, 100(12): 7289-7294, 2003); and hepatitis C virus (Pfeiffer and Kirkegaard, J. Virol. 79(4): 2346-2355, 2005). A number of other persistent RNA viruses, such as hepatitis and HIV, have evolved resistance to existing antiviral drugs: hepatitis B virus (lamivudine, tenofovir, entecavir) doi: 10/1002/hep22900; hepatitis C virus (telaprevir, BILN2061, ITMN-191, SCh6, boceprevir, AG-021541, ACH-806) doi: 10.1002/hep.22549; and HIV (many drug resistance mutations) hivb.standford.edu. The embodiments disclosed herein may be used to detect such variants among others.

Aside from drug resistance, there are a number of clinically relevant mutations that could be detected with the embodiments disclosed herein, such as persistent versus acute infection in LCMV (doi: 10.1073/pnas. 1019304108), and increased infectivity of Ebola (Diehl et al. Cell. 2016, 167(4): 1088-1098.

As described herein elsewhere, closely related microbial species (e.g., having only a single nucleotide difference in a given target sequence) may be distinguished by introduction of a synthetic mismatch in the nucleic acid component molecule.

### Monitoring Microbe Outbreaks

In one embodiment, a TnpB system or methods of use thereof as described herein may be used to determine the evolution of a pathogen outbreak. The method may comprise detecting one or more target sequences from a plurality of samples from one or more subjects, wherein the target sequence is a sequence from a microbe causing the outbreaks. Such a method may further comprise determining a pattern of pathogen transmission, or a mechanism involved in a disease outbreak caused by a pathogen.

The pattern of pathogen transmission may comprise continued new transmissions from the natural reservoir of the pathogen or subject-to-subject transmissions (e.g., human-to-human transmission) following a single transmission from the natural reservoir or a mixture of both. In one embodiment, the pathogen transmission may be bacterial or viral transmission, in such case, the target sequence is preferably a microbialgenome or fragments thereof. In one embodiment, the pattern of the pathogen transmission is the early pattern of the pathogen transmission, i.e., at the beginning of the pathogen outbreak. Determining the pattern of the pathogen transmission at the beginning of the outbreak increases likelihood of stopping the outbreak at the earliest possible time thereby reducing the possibility of local and international dissemination.

Determining the pattern of the pathogen transmission may comprise detecting a pathogen sequence according to the methods described herein. Determining the pattern of the pathogen transmission may further comprise detecting shared intra-host variations of the pathogen sequence between the subjects and determining whether the shared intra-host variations show temporal patterns. Patterns in observed intrahost and interhost variation provide important insight about transmission and epidemiology (Gire, et al., 2014).

Detection of shared intra-host variations between the subjects that show temporal patterns is an indication of transmission links between subject (in particular between humans) because it can be explained by subject infection from multiple sources (superinfection), sample contamination recurring mutations (with or without balancing selection to reinforce mutations), or co-transmission of slightly divergent viruses that arose by mutation earlier in the transmission chain (Park, et al., Cell 161(7): 1516-1526, 2015). Detection of shared intra-host variations between subjects may comprise detection of intra-host variants located at common single nucleotide polymorphism (SNP) positions. Positive detection of intra-host variants located at common (SNP) positions is indicative of superinfection and contamination as primary explanations for the intra-host variants. Superinfection and contamination can be parted on the basis of SNP frequency appearing as inter-host variants (Park, et al., 2015). Otherwise, superinfection and contamination can be ruled out. In this latter case, detection of shared intra-host variations between subjects may further comprise assessing the frequencies of synonymous and nonsynonymous variants and comparing the frequency of synonymous and nonsynonymous variants to one another. A nonsynonymous mutation is a mutation that alters the amino acid of the protein, likely resulting in a biological change in the microbe that is subject to natural selection. Synonymous substitution does not alter an amino acid sequence. Equal frequency of synonymous and nonsynonymous variants is indicative of the intra-host variants evolving neutrally. If frequencies of synonymous and nonsynonymous variants are divergent, the intra-host variants are likely to be maintained by balancing selection. If frequencies of synonymous and nonsynonymous variants are low, this is indicative of recurrent mutation. If frequencies of synonymous and nonsynonymous variants are high, this is indicative of co-transmission (Park, et al., 2015).

Like Ebola virus, Lassa virus (LASV) can cause hemorrhagic fever with high case fatality rates. Andersen et al. generated a genomic catalog of almost 200 LASV sequences from clinical and rodent reservoir samples (Andersen, et al., Cell Volume 162, Issue 4, p 738-750, 13 Aug. 2015). Andersen et al. show that whereas the 2013-2015 EVD epidemic is fueled by human-to-human transmissions, LASV infections mainly result from reservoir-to-human infections. Andersen et al. elucidated the spread of LASV across West Africa and show that this migration was accompanied by changes in LASV genome abundance, fatality rates, codon adaptation, and translational efficiency. The method may further comprise phylogenetically comparing a first pathogen sequence to a second pathogen sequence, and determining whether there is a phylogenetic link between the first and second pathogen sequences. The second pathogen sequence may be an earlier reference sequence. If there is a phylogenetic link, the method may further comprise rooting the phylogeny of the first pathogen sequence to the second pathogen sequence. Thus, it is possible to construct the lineage of the first pathogen sequence. (Park, et al., 2015).

The method may further comprise determining whether the mutations are deleterious or adaptive. Deleterious mutations are indicative of transmission-impaired viruses and dead-end infections, thus normally only present in an individual subject. Mutations unique to one individual subject are those that occur on the external branches of the phylogenetic tree, whereas internal branch mutations are those present in multiple samples (i.e., in multiple subjects). Higher rate of nonsynonymous substitution is a characteristic of external branches of the phylogenetic tree (Park, et al., 2015).

In internal branches of the phylogenetic tree, selection has had more opportunity to filter out deleterious mutants. Internal branches, by definition, have produced multiple descendent lineages and are thus less likely to include mutations with fitness costs. Thus, lower rate of nonsynonymous substitution is indicative of internal branches (Park, et al., 2015).

Synonymous mutations, which likely have less impact on fitness, occurred at more comparable frequencies on internal and external branches (Park, et al., 2015).

By analyzing the sequenced target sequence, such as viral genomes, it is possible to discover the mechanisms responsible for the severity of the epidemic episode such as during the 2014 Ebola outbreak. For example, Gire et al. made a phylogenetic comparison of the genomes of the 2014 outbreak to all 20 genomes from earlier outbreaks suggests that the 2014 West African virus likely spread from central Africa within the past decade. Rooting the phylogeny using divergence from other ebolavirus genomes was problematic (6, 13). However, rooting the tree on the oldest outbreak revealed a strong correlation between sample date and root-to-tip distance, with a substitution rate of 8×10⁻⁴ per site per year (13). This suggests that the lineages of the three most recent outbreaks all diverged from a common ancestor at roughly the same time, around 2004, which supports the hypothesis that each outbreak represents an independent zoonotic event from the same genetically diverse viral population in its natural reservoir. They also found out that the 2014 EBOV outbreak might be caused by a single transmission from the natural reservoir, followed by human-to-human transmission during the outbreak. Their results also suggested that the epidemic episode in Sierra Leon might stem from the introduction of two genetically distinct viruses from Guinea around the same time (Gire, et al., 2014).

It has been also possible to determine how the Lassa virus spread out from its origin point, in particular thanks to human-to-human transmission and even retrace the history of this spread 400 years back (Andersen, et al., Cell 162(4): 738-50, 2015).

In relation to the work needed during the 2013-2015 EBOV outbreak and the difficulties encountered by the medical staff at the site of the outbreak, and more generally, the method of the invention makes it possible to carry out sequencing using fewer selected probes such that sequencing can be accelerated, thus shortening the time needed from sample taking to results procurement. Further, kits and systems can be designed to be usable on the field so that diagnostics of a patient can be readily performed without need to send or ship samples to another part of the country or the world.

In any method described above, sequencing the target sequence or fragment thereof may be used any of the sequencing processes described above. Further, sequencing the target sequence or fragment thereof may be a near-real-time sequencing. Sequencing the target sequence or fragment thereof may be carried out according to previously described methods (Experimental Procedures: Matranga et al., 2014; and Gire, et al., 2014). Sequencing the target sequence or fragment thereof may comprise parallel sequencing of a plurality of target sequences. Sequencing the target sequence or fragment thereof may comprise Illumina sequencing.

Analyzing the target sequence or fragment thereof that hybridizes to one or more of the selected probes may be an identifying analysis, wherein hybridization of a selected probe to the target sequence or a fragment thereof indicates the presence of the target sequence within the sample.

Currently, primary diagnostics are based on the symptoms a patient has. However, various diseases may share identical symptoms so that diagnostics rely much on statistics. For example, malaria triggers flu-like symptoms: headache, fever, shivering, joint pain, vomiting, hemolytic anemia, jaundice, hemoglobin in the urine, retinal damage, and convulsions. These symptoms are also common for septicemia, gastroenteritis, and viral diseases. Amongst the latter, Ebola hemorrhagic fever has the following symptoms fever, sore throat, muscular pain, headaches, vomiting, diarrhea, rash, decreased function of the liver and kidneys, internal and external hemorrhage.

When a patient is presented to a medical unit, for example in tropical Africa, basic diagnostics will conclude to malaria because statistically, malaria is the most probable disease within that region of Africa. The patient is consequently treated for malaria although the patient might not actually have contracted the disease, and the patient ends up not being correctly treated. This lack of correct treatment can be life-threatening especially when the disease the patient contracted presents a rapid evolution. It might be too late before the medical staff realizes that the treatment given to the patient is ineffective and comes to the correct diagnostics and administers the adequate treatment to the patient.

The method of the invention provides a solution to this situation. Indeed, because the number of nucleic acid component molecules can be dramatically reduced, this makes it possible to provide on a single chip selected probes divided into groups, each group being specific to one disease, such that a plurality of diseases, e.g., viral infection, can be diagnosed at the same time. Thanks to the invention, more than 3 diseases can be diagnosed on a single chip, preferably more than 4, 5, 6, 7, 8, 9, 10, 11, 12, 13, 14, 15, 16, 17, 18, 19, 20 diseases at the same time, preferably the diseases that most commonly occur within the population of a given geographical area. Since each group of selected probes is specific to one of the diagnosed diseases, a more accurate diagnosis can be performed, thus diminishing the risk of administering the wrong treatment to the patient.

In other cases, a disease such as a viral infection may occur without any symptoms, or had caused symptoms but dissipated before the patient is presented to the medical staff. In such cases, either the patient does not seek any medical assistance or the diagnostics is complicated due to the absence of symptoms on the day of the presentation.

The present invention may also be used in concert with other methods of diagnosing disease, identifying pathogens and optimizing treatment based upon detection of nucleic acids, such as mRNA in crude, non-purified samples.

The method of the invention also provides a powerful tool to address this situation. Indeed, since a plurality of groups of selected nucleic acid component molecules, each group being specific to one of the most common diseases that occur within the population of the given area, are comprised within a single diagnostic, the medical staff only need to contact a biological sample taken from the patient with the chip. Reading the chip reveals the diseases the patient has contracted.

In some cases, the patient is presented to the medical staff for diagnostics of particular symptoms. The method of the invention makes it possible not only to identify which disease causes these symptoms but at the same time determine whether the patient suffers from another disease he was not aware of.

This information might be of utmost importance when searching for the mechanisms of an outbreak. Indeed, groups of patients with identical viruses also show temporal patterns suggesting a subject-to-subject transmission links.

### Example Microbes

The embodiment disclosed herein may be used to detect a number of different microbes. The term microbe as used herein includes bacteria, fungus, protozoa, parasites and viruses.

### Bacteria

The following provides an example list of the types of microbes that might be detected using the embodiments disclosed herein. In certain example embodiments, the microbe is a bacterium. Examples of bacteria that can be detected in accordance with the disclosed methods include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginale Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Epidermophyton floccosum, Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Microsporum canis, Moraxella catarrhalis, Morganella sp., Mobiluncus sp., Micrococcus sp., Mycobacterium sp. (such as Mycobacterium leprae, Mycobacterium tuberculosis, Mycobacterium paratuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Pityrosporum orbiculare (Malassezia furfur), Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Trichophyton rubrum, T. mentagrophytes, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia among others.

### Fungi

In certain example embodiments, the microbe is a fungus or a fungal species. Examples of fungi that can be detected in accordance with the disclosed methods include without limitation any one or more of (or any combination of), Aspergillus, Blastomyces, Candidiasis, Coccidiodomycosis, Cryptococcus neoformans, Cryptococcus gatti, sp. Histoplasma sp. (such as Histoplasma capsulatum), Pneumocystis sp. (such as Pneumocystis jirovecii), Stachybotrys (such as Stachybotrys chartarum), Mucroymcosis, Sporothrix, fungal eye infections ringworm, Exserohilum, Cladosporium.

In certain example embodiments, the fungus is a yeast. Examples of yeast that can be detected in accordance with disclosed methods include without limitation one or more of (or any combination of), Aspergillus species (such as Aspergillus fumigatus, Aspergillus flavus and Aspergillus clavatus), Cryptococcus sp. (such as Cryptococcus neoformans, Cryptococcus gattii, Cryptococcus laurentii and Cryptococcus albidus), a Geotrichum species, a Saccharomyces species, a Hansenula species, a Candida species (such as Candida albicans), a Kluyveromyces species, a Debaryomyces species, a Pichia species, or combination thereof. In certain example embodiments, the fungi is a mold. Example molds include, but are not limited to, a Penicillium species, a Cladosporium species, a Byssochlamys species, or a combination thereof.

### Protozoa

In certain example embodiments, the microbe is a protozoa. Examples of protozoa that can be detected in accordance with the disclosed methods and devices include without limitation any one or more of (or any combination of), Euglenozoa, Heterolobosea, Diplomonadida, Amoebozoa, Blastocystic, and Apicomplexa. Example Euglenoza include, but are not limited to, Trypanosoma cruzi (Chagas disease), T. brucei gambiense, T. brucei rhodesiense, Leishmania braziliensis, L. infantum, L. mexicana, L. major, L. tropica, and L. donovani. Example Heterolobosea include, but are not limited to, Naegleria fowleri. Example Diplomonadids include, but are not limited to, Giardia intestinalis (G. lamblia, G. duodenalis). Example Amoebozoa include, but are not limited to, Acanthamoeba castellanii, Balamuthia madrillaris, Entamoeba histolytica. Example Blastocysts include, but are not limited to, Blastocystic hominis. Example Apicomplexa include, but are not limited to, Babesia microti, Cryptosporidium parvum, Cyclospora cayetanensis, Plasmodium falciparum, P. vivax, P. ovale, P. malariae, and Toxoplasma gondii.

### Parasites

In certain example embodiments, the microbe is a parasite. Examples of parasites that can be detected in accordance with disclosed methods include without limitation one or more of (or any combination of), an Onchocerca species and a Plasmodium species.

### Viruses

In certain example embodiments, the systems, devices, and methods, disclosed herein are directed to detecting viruses in a sample. The embodiments disclosed herein may be used to detect viral infection (e.g., of a subject or plant), or determination of a viral strain, including viral strains that differ by a single nucleotide polymorphism. The virus may be a DNA virus, a RNA virus, or a retrovirus. Non-limiting example of viruses useful with the present invention include, but are not limited to Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2. In certain example embodiments, the viral sequence may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyxovirus, Australian bat lyssavirus, Avian bornavirus, Avian metapneumovirus, Avian paramyxoviruses, penguin or Falkland Islandsvirus, BK polyomavirus, Bagaza virus, Banna virus, Bat herpesvirus, Bat sapovirus, Bear Canon mammarenavirus, Beilong virus, Betacoronavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwera virus, Caliciviridae virus. California encephalitis virus, Candiru virus, Canine distemper virus, Canine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyxovirus SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human genital-associated circular DNA virus-1, Human herpesvirus 1-8, Human immunodeficiency virus 1/2, Human mastadenovirus A-G, Human papillomavirus, Human parainfluenza virus 1-4, Human paraechovirus, Human picornavirus, Human smacovirus, Ikoma lyssavirus, Ilheus virus, Influenza A-C, Ippy mammarenavirus, Irkut virus, J-virus, JC polyomavirus, Japanese encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2\0.225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O′nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Porcine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence. Examples of RNA viruses that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In certain example embodiments, the virus may be a plant virus selected from the group comprising Tobacco mosaic virus (TMV), Tomato spotted wilt virus (TSWV), Cucumber mosaic virus (CMV), Potato virus Y (PVY), the RT virus Cauliflower mosaic virus (CaMV), Plum pox virus (PPV), Brome mosaic virus (BMV), Potato virus X (PVX), Citrus tristeza virus (CTV), Barley yellow dwarf virus (BYDV), Potato leafroll virus (PLRV), Tomato bushy stunt virus (TBSV), rice tungro spherical virus (RTSV), rice yellow mottle virus (RYMV), rice hoja blanca virus (RHBV), maize rayado fino virus (MRFV), maize dwarf mosaic virus (MDMV), sugarcane mosaic virus (SCMV), Sweet potato feathery mottle virus (SPFMV), sweet potato sunken vein closterovirus (SPSVV), Grapevine fanleaf virus (GFLV), Grapevine virus A (GVA), Grapevine virus B (GVB), Grapevine fleck virus (GFkV), Grapevine leafroll-associated virus-1, -2, and -3, (GLRaV-1, -2, and -3), Arabis mosaic virus (ArMV), or Rupestris stem pitting-associated virus (RSPaV). In a preferred embodiment, the target RNA molecule is part of said pathogen or transcribed from a DNA molecule of said pathogen. For example, the target sequence may be comprised in the genome of an RNA virus. It is further preferred that TnpB protein hydrolyzes said target RNA molecule of said pathogen in said plant if said pathogen infects or has infected said plant. It is thus preferred that the TnpB system is capable of cleaving the target RNA molecule from the plant pathogen both when the TnpB system (or parts needed for its completion) is applied therapeutically, i.e., after infection has occurred or prophylactically, i.e. before infection has occurred.

In certain example embodiments, the virus may be a retrovirus. Example retroviruses that may be detected using the embodiments disclosed herein include one or more of or any combination of viruses of the Genus Alpharetrovirus, Betaretrovirus, Gammaretrovirus, Deltaretrovirus, Epsilonretrovirus, Lentivirus, Spumavirus, or the Family Metaviridae, Pseudoviridae, and Retroviridae (including HIV), Hepadnaviridae (including Hepatitis B virus), and Caulimoviridae (including Cauliflower mosaic virus).

In certain example embodiments, the virus is a DNA virus. Example DNA viruses that may be detected using the embodiments disclosed herein include one or more of (or any combination of) viruses from the Family Myoviridae, Podoviridae, Siphoviridae, Alloherpesviridae, Herpesviridae (including human herpes virus, and Varicella Zozter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidovirus, among others. In one embodiment, a method of diagnosing a species-specific bacterial infection in a subject suspected of having a bacterial infection is described as obtaining a sample comprising bacterial ribosomal ribonucleic acid from the subject; contacting the sample with one or more of the probes described, and detecting hybridization between the bacterial ribosomal ribonucleic acid sequence present in the sample and the probe, wherein the detection of hybridization indicates that the subject is infected with Escherichia coli, Klebsiella pneumoniae, Pseudomonas aeruginosa, Staphylococcus aureus, Acinetobacter baumannii, Candida albicans, Enterobacter cloacae, Enterococcus faecalis, Enterococcus faecium, Proteus mirabilis, Staphylococcus agalactiae, or Staphylococcus maltophilia or a combination thereof.

### Coronavirus

Systems and methods of the presently disclosed invention are designed to detect coronavirus, in an aspect, the target sequence is the 2019-nCOV, also referred to herein as SARS-CoV-2, which causes COVID-19. The coronavirus is a positive-sense single stranded RNA family of viruses, infecting a variety of animals and humans. SARS-COV is one type of coronavirus infection, as well as MERS-COV. Detection of one or more coronaviruses are envisioned, including the SARS-COV-2 detected in Wuhan City. Sequences of the SARS-COV-2 are available at GISAID accession no. EPI_ISL_402124 and EPI_ISL_402127-402130, and described in DOI: 10.1101/2020.01.22.914952. Further deposits of the SARS-COV2 are deposited in the GISAID platform include EP_ISL 402119-402121 and EP_ISL_402123-402124; see also GenBank Accession No. MN908947.3. In an aspect, one may use known SARS and SARS-related coronaviruses or other viruses from one or more hosts to generate a non-redundant alignment. Related viruses can be found, for example in bats.

In one embodiment, the systems are designed to comprise at least one highly active nucleic acid component polynucleotide which is designed according to the methods disclosed herein. In a preferred embodiment, the nucleic acid component polynucleotide binds to at least one target sequence that is a unique coronavirus genomic sequence, thereby identifying the presence of coronavirus to the exclusion of other viruses. The systems and methods can be designed to detect a plurality of respiratory infections or viral infections, including coronavirus.

In an aspect the at least one nucleic acid component polynucleotide binds to a coronavirus sequence encoding a polypeptide that is immunostimulatory to a host immune system. Immunostiumulatory polypeptides have the ability to enhance, stimulate, or increase response of the immune system, typically by inducing the activation or activity of a components of the immune system (e.g., an immune cell). In embodiments, the immunostimulatory polypeptide contributes to immune-mediated disease in the host. In an aspect, the host is a mammal, for example, a human, a bat, or a pangolin, that may be infected by a coronavirus. Cyranoski, D. Did pangolins spread the China coronavirus to people? Nature, 7 Feb. 2020. In one embodiment, the nucleic acid component polynucleotide can be designed to detect SARS-COV-2 or a variant thereof in meat, live anmials and humans so that testing can be performed, for example at markets and other public places where sources of contamination can arise.

Gene targets may comprise ORFlab, N protein, RNA-dependent RNA polymerase (RdRP), E protein, ORF1b-nsp14, Spike glycoprotein(S), or pancorona targets. Molecular assays have been under development and can be used as a starting point to develop nucleic acid component molecules for the methods and systems described herein. See, “Diagnostic detection of 2019-nCOV by real-time RT-PCR” Charité, Berlin Germany S (17 Jan. 2020)′ Detection of 2019 novel coronavirus (2019-nCOV) in suspected human cases by RT-PCR-Hong Kong University (23 Jan. 2020); PCR and sequencing protocol for 2019-nCOV-Department of Medical Sciences, Ministry of Public Health, Thailand (updated 28 Jan. 2020); PCR and sequencing protocols for 2019-nCOV-National Institute of Infectious Diseases Japan (24 Jan. 2020); US CDC panel primer and probes-U.S. CDC, USAV-U.S. CDC, USA (28 Jan. 2020); China CDC Primers and probes for detection 2019-nCOV (24 Jan. 2020), incorporated in their entirety by reference. Further, the nucleic acid component molecule design may exploit differences or similarities with SARS-COV. Researchers have recently identified simialrities and fifrferences between 2019-nCOV and SARS-COV. “Coronavirus Genome Annotation Reveals Amino Acid Differences with Other SARS Viruses,” genomeweb, Feb. 10, 2020. For example, nucleic acid component molecules based on the 8a protein, which was present in SARS-CoV but absent in SARS-COV-2, can be utilized to differentiate between the viruses. Similarly, the 8b and 3b proteins have different lengths in SARS-COV and SARS-COV-2 and can be utilized to design nucleic acid component molecules to detect non-overlapping proteins of nucleotides encoding in the two viruses. Wu et al., Genome Composition and Divergence of the Novel Coronavirus (2019-nCOV) Originating in China, Cell Host & Microbe (2020), DOI: 10.1016/j.chom.2020.02.001, incorporated herein by reference, including all supplemental information, in particular Table S1. Mutations may also be detected, with nucleic acid component and/or primers designed specifically to detect, for example, changes in the SARS-COV-2 virus. In an embodiment, the nucleic acid component or primer can be designed to detect the D614G mutation in the SARS-COV-2 spike protein. See, Korber et al., Cell 182, 812-827 (2020); doi: 10.1016/j.cell.2020.06.043.Other mutations in the spike protein can be designed utilizing the COVID-19 viral genome analysis pipeline available at cov.lanl.gov. Further resources to design primers and nucleic acid components to detect coronavirus or coronavirus mutations can be found at Starr, et al, “Deep Mutational Scanning of SARS-COV-2 Receptor Binding Domain Reveals Constraints in Folding and ACE2 Binding,” Cell, 182, 1-16 (2020); doi: 10.1016/j.cell.2020.08.012.

The systems and methods of detection can be used to identify single nucleotide variants, detection based on rRNA sequences, screening for drug resistance, monitoring microbe outbreaks, genetic perturbations, and screening of environmental samples, as described in PCT/US2018/054472 filed Oct. 22, 2018 at [0183]-[0327], incorporated herein by reference.

In certain example embodiments, the systems, devices, and methods disclosed herein may be used for biomarker detection. For example, the systems, devices and method disclosed herein may be used for SNP detection and/or genotyping. The systems, devices and methods disclosed herein may be also used for the detection of any disease state or disorder characterized by aberrant gene expression. Aberrant gene expression includes aberration in the gene expressed, location of expression and level of expression. Multiple transcripts or protein markers related to cardiovascular, immune disorders, and cancer among other diseases may be detected. In certain example embodiments, the embodiments disclosed herein may be used for cell free DNA detection of diseases that involve lysis. In certain example embodiments, the embodiments could be utilized for faster and more portable detection for pre-natal testing of cell-free DNA. The embodiments disclosed herein may be used for screening panels of different SNPs associated with, among others, different coronaviruses, evolving SARS-COV2, and other related respiratory viral infections. As described herein elsewhere, closely related genotypes/alleles or biomarkers (e.g., having only a single nucleotide difference in a given target sequence) may be distinguished by introduction of a synthetic mismatch in the nucleic acid component molecule.

In an aspect, the invention relates to a method for detecting target nucleic acids in samples, comprising: distributing a sample or set of samples into one or more individual discrete volumes, the individual discrete volumes comprising a TnpB system according to the invention as described herein; incubating the sample or set of samples under conditions sufficient to allow binding of the one or more nucleic acid component molecules to one or more target molecules; activating the TnpB protein via binding of the one or more nucleic acid component molecules to the one or more target molecules, wherein activating the TnpB protein results in modification of the RNA-based masking construct such that a detectable positive signal is generated; and detecting the detectable positive signal, wherein detection of the detectable positive signal indicates a presence of one or more target molecules in the sample.

The sensitivity of the assays described herein are well suited for detection of target nucleic acids in a wide variety of biological sample types, including sample types in which the target nucleic acid is dilute or for which sample material is limited. Methods for field deployable and rapid diagnostic assays can be optimized for the type of sample material utilized and can be adapted from approaches used for other assays known in the art. See, e.g., Myhrvold et al., 2018. Biomarker screening may be carried out on a number of sample types including, but not limited to, saliva, urine, blood, feces, sputum, and cerebrospinal fluid. The embodiments disclosed herein may also be used to detect up- and/or down-regulation of genes. For example, a sample may be serially diluted such that only over-expressed genes remain above the detection limit threshold of the assay.

In one embodiment, the present invention provides steps of obtaining a sample of biological fluid (e.g., urine, blood plasma or serum, sputum, cerebral spinal fluid), and extracting the DNA or RNA. The mutant nucleotide sequence to be detected, may be a fraction of a larger molecule or can be present initially as a discrete molecule.

In embodiments, DNA is isolated from plasma/serum of a cancer patient. For comparison, DNA samples isolated from neoplastic tissue and a second sample may be isolated from non-neoplastic tissue from the same patient (control), for example, lymphocytes. The non-neoplastic tissue can be of the same type as the neoplastic tissue or from a different organ source. In one embodiment, blood samples are collected and plasma immediately separated from the blood cells by centrifugation. Serum may be filtered and stored frozen until DNA/RNA extraction.

In an aspect, sample preparation can comprise methods as disclosed herein to circumvent other RNA extraction methods and can be used with standard amplification techniques such as RT-PCR as well as the TnpB detection methods disclosed herein. In an aspect, the method may comprise a one-step extraction-free RNA preparation method that can be used with samples tested for coronavirus, which may be, in an aspect, a RT-qPCR testing method, a lateral flow detection method or other TnpB detection method disclosed herein. Advantageously, the RNA extraction method can be utilized directly with other testing protocols. In an aspect, the method comprises use of a nasopharyngeal swab, nasal saline lavage, or other nasal sample (e.g., anterior nasal swab) with Quick Extract™ DNA Extraction Solution (QE09050), Lucigen, or QuickExtract Plant DNA Extraction Solution, Lucigen. In an aspect, the sample is diluted 2:1, 1:1 or 1:2 sample: DNA extraction solution. The sample: extraction mix is incubated at about 90° C. to about 98° C., preferably about 95° C. In another aspect, incubation is performed at between about 20° C. to about 90° C., about 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85 86, 87, 88, 89 or 90° C. The incubation period can be about 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes, preferably about 4 to 6 minutes, or about 5 minutes. Incubation time and temperature may vary depending on sample size and quality, and incubation time may increase if using lower temperature. Current CDC Real-Time RT-PCR Diagnostic Panel are as described at fda.gov/media/134922/download, “CDC 2019-Novel Coronavirus (2019-nCOV) Real-Time RT-PCR Diagnostic Panel.” In one embodiment, the DNA extraction solution can remain with the sample subsequent to incubation and be utilized in the next steps of detection methods. In an aspect, the detection method is an RT-qPCR reaction and the extraction solution is kept at a concentration of less than 10%, 9%, 8%, 7%, 6%, 5%, 4%, 3% of the reaction mixture, where the reaction mixture comprises the detection reaction reagents, sample and extraction solution.

In one embodiment, a bead is utilized with particular embodiments of the invention and may be included with the extraction solution. The bead may be used to capture, concentrate or otherwise enrich for particular material. The bead may be magnetic, and may be provided to capture nucleic acid material. In another aspect, the bead is a silica bead. Beads may be utilized in an extraction step of the methods disclosed herein. Beads can be optionally used with the methods described herein, including with the one-pot methods that allow for concentration of viral nucleic acids from large volume samples, such as saliva or swab samples to allow for a single one-pot reaction method. Concentration of desired target molecules can be increased by about 10-fold, 50-fold, 100-fold, 200-fold, 500-fold, 800-fold, 1000-fold, 1500-fold, 2000-fold, 2500-fold, 3000-fold, or more.

Magnetic beads in a PEG and salt solution are preferred in an aspect, and in embodiments bind to viral RNA and/or DNA which allows for concentration and lysis concurrently. Silica beads can be used in another aspect. Capture moieties such as oligonucleotide functionalized beads are envisioned for use. The beads may be using with the extraction reagents, allowed to incubate with a sample and the lysis/extraction buffer, thereby concentrating target molecules on the beads. Extraction can be performed as described elsewhere herein, at 22° C.-60° C., with subsequent isothermal amplification and/or TnpB detection performed under conditions as described elsewhere herein. When used with a cartridge device detailed elsewhere herein, a magnet can be activated and the beads collected, with optional flushing of the extraction buffer and one or more washes performed. Advantageously, the beads can be used in the one-pot methods and systems without additional washings of the beads, allowing for a more efficient process without increased risks of contamination in multi-step processes. Beads can be utilized with the isothermal amplifications detailed herein, and the beads can flow into an amplification chamber of the cartridge or be maintained in the pot for the amplification step. Upon heating, nucleic acid can be released off the beads.

In certain example embodiments, target nucleic acids are detected directly from a crude or unprocessed sample, such as blood, serum, saliva, cebrospinal fluid, sputum, or urine. In certain example embodiments, the target nucleic acid is cell free DNA.

### Kits

In one aspect, the invention provides kits containing any one or more of the elements disclosed in the above methods and compositions. In one aspect, the invention provides a kit comprising one or more of the components described herein. In one embodiment, the kit comprises the compositions herein and instructions for using the kit. In one embodiment, the kit comprises a vector system and instructions for using the kit. In one embodiment, the kit comprises a delivery system and instructions for using the kit. In one embodiment, the kit comprises a vector system and instructions for using the kit. Elements may be provided individually or in combinations, and may be provided in any suitable container, such as a vial, a bottle, or a tube. The kits may include the nucleic acid component and optionally an unbound protector strand as described herein. The kits may include the nucleic acid component with a protector strand bound to at least partially to a reprogrammable spacer portion of the nucleic acid component sequence (i.e., pnucleic acid component). Thus, the kits may include the pnucleic acid component in the form of a partially double stranded nucleotide sequence as described here. In one embodiment, the kit includes instructions in one or more languages, for example in more than one language. The instructions may be specific to the applications and methods described herein.

In one embodiment, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g., in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In one embodiment, the buffer is alkaline. In one embodiment, the buffer has a pH from about 7 to about 10. In one embodiment, the kit comprises one or more oligonucleotides corresponding to a nucleic acid component scaffold, reprogrammable sequence for insertion into a vector so as to operably link the nucleic acid component sequence and a regulatory element. In one embodiment, the kit comprises a homologous recombination template polynucleotide. In one embodiment, the kit comprises one or more of the vectors and/or one or more of the polynucleotides described herein. The kit may advantageously allow to provide all elements of the systems of the invention.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A non-naturally occurring, engineered composition comprising a) a TnpB polypeptide, a MazE polypeptide or domain and a nucleic acid component comprising a scaffold and a reprogrammable spacer sequence, the nucleic acid component capable of forming a complex with the TnpB polypeptide and directing sequence-specific binding of a target polynucleotide, and wherein the MazE polypeptide or domain is linked to or otherwise capable of complexing with the TnpB polypeptide and/or nucleic acid component.

2. The composition of claim 1, wherein the TnpB polypeptide comprises about 200 to about 500 amino acids.

3. The composition of claim 1, wherein the reprogrammable spacer sequence comprises a spacer of 10 nucleotides to 30 nucleotides in length.

4. The composition of claim 1, wherein the nucleic acid component comprises a scaffold of about 80 to 200 nucleotides in length.

5. The composition of any of the previous claims, wherein the complex binds a target adjacent motif (TAM) sequence 5′ of the target polynucleotide.

6. The composition of claim 5, wherein the TAM sequence comprises TCA.

7. The composition of claim 5, wherein the TAM sequence comprises TTCAN.

8. The composition of any of the previous claims, wherein the target polynucleotide is DNA.

9. The composition of any one of the preceding claims, further comprising a homologous recombination donor template comprising a donor sequence for insertion into a target polynucleotide.

10. The composition of any of the previous claims, further comprising a functional domain associated with the TnpB polypeptide.

11. The composition of claim 10, wherein the functional domain is a transposase, an integrase, a nucleobase deaminase, a reverse transcriptase, a recombinase, an integrase, a topoisomerase, a retrotransposon, phosphatase, polymerase, ligase, helitron, a helicase, a methylase, a demethylase, a translation activater, a translation repressor, a transcription activator, a transcription repressor, a transcription release factor, a chromatin modifier, a histone modifier, a nuclease.

12. The composition of any one of the preceding claims, wherein the TnpB polypeptide and MazE polypeptide are selected from Table 1.

13. A vector system comprising one or more vectors encoding the TnpB polypeptide and the nucleic acid component of any of the preceding claims.

14. An engineered cell comprising the composition of any of claims 1 to 13.

15. A method of modifying target polynucleotide sequences in a cell, comprising introducing into the cell the composition of any of claims 1 to 13, wherein the TnpB complex makes one or more modifications to one or more target polynucleotide sequences.

16. The method of claim 15, wherein the one or more modifications comprises cleaving the one or more target polynucleotides.

17. The method of claim 16, wherein the cleaving results in a 5′ overhang.

18. The method of claim 17, wherein the cleaving occurs distal to a target-adjacent motif.

19. The method of claim 18, wherein the cleaving occurs at the site of the spacer annealing site or 3′ of the target sequence.

20. The method of claim 15, wherein the polypeptide and/or nucleic acid components are provided via one or more polynucleotides encoding the polypeptides and/or nucleic acid component(s), and wherein the one or more polynucleotides are operably configured to express the TnpB polypeptide and/or the nucleic acid components.

21. The method of claim 15, wherein the one or more modifications comprise one or more mutations

22. The method of claim 21, wherein the one or more mutations include substitutions, deletions, and insertions.

23. An engineered, non-naturally occurring composition comprising:
a. a TnpB polypeptide, wherein the TnpB polypeptide is catalytically inactive,
b. a nucleotide deaminase associated with or otherwise capable of forming a complex with the TnpB polypeptide,
c. a MazE polypeptide or domain; and
d. an nucleic acid component molecule capable of forming a complex with the TnpB protein and directing site-specific binding at a target sequence.

24. The composition of claim 23, wherein the TnpB and MazE is selected from Table 1.

25. The composition of claim 24, wherein the nucleotide deaminase is an adenosine deaminase or a cytidine deaminase.

26. One or more polynucleotides encoding one or more components of the composition of any one of claim 23 or 24.

27. One or more vectors encoding the one or more polynucleotides of claim 26.

28. A cell or progeny thereof genetically engineered to express one or more components of the composition of any one of claims 1 to 27.

29. A method of editing nucleic acids in target polynucleotides comprising delivering the composition of claim 23 or 24, the one or more polynucleotides of claim 26, or one or more vectors of claim 27 to a cell or population of cells comprising the target polynucleotides.

30. The method of claim 29, wherein the target polynucleotides are target sequences within genomic DNA.

31. The method of claim 29 or 30, wherein the target polynucleotide is edited at one or more bases to introduce a G→A or C→T mutation.

32. An isolated cell or progeny thereof comprising one or more base edits made using the method of any one of claims 30 to 31.

33. An engineered, non-naturally occurring composition comprising:
a. a catalytically dead TnpB polypeptide,
b. a reverse transcriptase associated with or otherwise capable of forming a complex with the TnpB polypeptide,
c. a MazE polypeptide or domain, and
d. an nucleic acid component molecule capable of forming a complex with the TnpB polypeptide and directing site-specific binding of the complex to a target sequence of a target polynucleotide, the guide molecule further comprising a donor template encoding a donor sequence for insertion into the target polynucleotide.

34. One or more polynucleotides encoding one or more components of the composition of claim 33.

35. One or more vectors encoding the one or more polynucleotides of claim 34.

36. A method of modifying target polynucleotides comprising;
delivering the composition of claim 33, the one or more polynucleotides of claim 34, or the one or more vectors of claim 35 to a cell, or population of cells, comprising the target polynucleotides, wherein the complex directs the reverse transcriptase to the target sequence and the reverse transcriptase facilitates insertion of a donor sequence encoded by the donor template from the ωRNA component molecule into the target polynucleotide.

37. The method of claim 36, wherein insertion of the donor sequence:
a. introduces one or more base edits;
b. corrects or introduces a premature stop codon;
c. disrupts a splice site;
d. inserts or restores a splice site;
e. inserts a gene or gene fragment at one or both alleles of the target polynucleotide; or;
f. a combination thereof.

38. An isolated cell or progeny thereof comprising the modifications made using the method of claim 36 or 37.

39. An engineered, non-naturally occurring composition comprising:
a. a TnpB polypeptide,
b. a non-LTR retrotransposon protein associated with or otherwise capable of forming a complex with the TnpB polypeptide,
c. a MazE polypeptide or domain,
d. a nucleic acid component molecule capable of forming a complex with the TnpB polypeptide and directing site-specific binding of the complex to a target sequence of a target polynucleotide;
e. and a donor template encoding a donor sequence for insertion into the target polynucleotide and located between two binding elements capable of forming a complex with the non-LTR retrotransposon protein.

40. The composition of claim 39 wherein the TnpB polypeptide is fused to the N-terminus of the non-LTR retrotransposon protein.

41. The composition of claim 39 or 40, wherein the TnpB polypeptide is engineered to have nickase activity.

42. The composition of claim 41, wherein the nucleic acid component molecule directs the fusion protein to a target sequence 5′ of the targeted insertion site, and wherein the TnpB polypeptide generates a strand break at the targeted insertion site.

43. The composition of claim 41, wherein the nucleic acid component molecule directs the fusion protein to a target sequence 3′ of the targeted insertion site, and wherein the TnpB polypeptide generates a strand break at the targeted insertion site.

44. The composition of claim 41, wherein the donor polynucleotide further comprises a homology region to the target sequence on the 5′ end of the donor construct, the 3′ end of the donor construct, or both.

45. The composition of claim 44, wherein the homology region is from 8 to 25 base pairs.

46. One or more polynucleotides encoding one or more components of the composition of any one of claims 41 to 45.

47. One or more vectors comprising the one or more polynucleotides of claim 46.

48. A method of modifying target polynucleotides comprising;
delivering the composition of any one of claims 41 to 45, the one or more polynucleotides of claim 45, or one or more vectors of claim 47 to a cell or population of cells comprising the target polynucleotides, wherein the complex directs the non-LTR retrotransposon protein to the target sequence and the non-LTR retrotransposon protein facilitates insertion of the donor polynucleotide sequence from the donor construct into the target polynucleotide.

49. The method of claim 48, wherein insertion of the donor sequence:
a. introduces one or more base edits;
b. corrects or introduces a premature stop codon;
c. disrupts a splice site;
d. inserts or restores a splice site;
e. inserts a gene or gene fragment at one or both alleles of the target polynucleotide; or;
f. a combination thereof.

50. An isolated cell or progeny thereof comprising the modifications made using the method of claim 48 or 49.

51. An engineered, non-naturally occurring composition comprising:
a. A TnpB polypeptide,
b. an integrase protein associated with or otherwise capable of forming a complex with the TnpB polypeptide, and optionally a reverse transcriptase, and
c. an nucleic acid component molecule capable of forming a complex with the TnpB polypeptide and directing site-specific binding of the complex to a target sequence of a target polynucleotide, the guide molecule further comprising a donor template encoding a donor sequence for insertion into the target polynucleotide and located between two binding elements capable of forming a complex with the integrase protein.

52. The composition of claim 51 wherein the TnpB polypeptide is fused to the integrase protein and optionally the reverse transcriptase.

53. The composition of claim 51 or 52, wherein the TnpB polypeptide is engineered to have nickase activity.

54. The composition of claim 53, wherein the nucleic acid component molecule directs the fusion protein to a target sequence, and wherein the TnpB polypeptide generates a nick at the targeted insertion site.

55. The composition of claim 53, wherein the donor polynucleotide further comprises a homology region to the target sequence on the 5′ end of the donor construct, the 3′ end of the donor construct, or both.

56. One or more polynucleotides encoding one or more components of the composition of any one of claims 53 to 55.

57. One or more vectors comprising the one or more polynucleotides of claim 56.

58. A method of modifying target polynucleotides comprising;
delivering the composition of any one of claims 51 to 55, the one or more polynucleotides of claim 56, or one or more vectors of claim 57 to a cell or population of cells comprising the target polynucleotides, wherein the complex directs the integrase protein to the target sequence and the integrase protein facilitates insertion of the donor polynucleotide sequence from the donor construct into the target polynucleotide.

59. The method of claim 58 wherein insertion of the donor sequence:
a. introduces one or more base edits;
b. corrects or introduces a premature stop codon;
c. disrupts a splice site;
d. inserts or restores a splice site;
e. inserts a gene or gene fragment at one or both alleles of the target polynucleotide; or;
f. a combination thereof.

60. An isolated cell or progeny thereof comprising the modifications made using the method of claim 58 or 59.

61. A composition for detecting the presence of a target polynucleotide in a sample, comprising:
one or more TnpB polypeptides possessing collateral activity;
at least one nucleic acid component comprising a sequence capable of binding a target polynucleotide and designed to form a complex with the one or more TnpB polypeptides;
a MazE polypeptide or domain, and
a detection construct comprising a polynucleotide component, wherein the TnpB polypeptides exhibit collateral nuclease activity and cleaves the polynucleotide component of the detection construct once activated by the target sequence; and
optionally, isothermal amplification reagents.

62. The composition of claim 61, wherein the TnpB and MazE is selected from Table 1.

63. The composition of claim 62, wherein the one or more TnpB polypeptides possess nuclease activity, in the temperature range of 45° C. to 60° C.

64. The composition of claim 62, wherein the isothermal amplification reagents are loop-mediated isothermal amplification (LAMP) reagents.

65. The composition of claim 64, wherein the LAMP reagents comprise LAMP primers.

66. The composition of any one of the claims 61 to 65, further comprising one or more additives to increase reaction specificity or kinetics.

67. The composition of any one of claims 61 to 66, further comprising polynucleotide binding beads.

68. A method for detecting polynucleotides in a sample, the method comprising;
contacting one or more target sequences with a TnpB polypeptide, at least one nucleic acid component capable of forming a complex with the TnpB polypeptide and direct sequence-specific binding to one or more target polynucleotides and a detection construct, wherein the TnpB polypeptide exhibits collateral nuclease activity and cleaves the detection construction once activated by the one or more target sequences; and
detecting a signal from cleavage of the detection construction thereby detecting the one or more target polynucleotides.

69. The method of claim 68, further comprising amplifying the target polynucleotides using isothermal amplification prior to the contacting step.

70. The method of claim 69, wherein detection of amplified target polynucleotides by binding of the target polynucleotides to the TnpB complex occurs in the temperature range of 45° C. to 60° C.

71. The method of claim 68, wherein the target polynucleotide is detected in one hour or less.


SEQUENCE LISTING (239 sequences)

SEQ ID NO: 1 - protein, 356 aa, synthetic construct
  REGION 1..356: note = Synthetic
VKLSFNISSEKAKKILSTVWLFKIACHRILSKSKEIKDMLVSSKVAWIKMFKNHAYEIIPNKRYSYGVVYLVYGIWESARRLRINYDDVELSDWLFIQHYEREVNGNVIRVYEDGSVLVTTYGYDGSKDRILVKAKPNKGQAELLNSIVESKEKYMPKLIIRDYGVRDDKLYVRGEVHISISYDFYLKHVKRYNEPKGSLIGGVDVNTDRINLAIIDEDGKLRDVKTFWFREVTARGYPRRRAWSIIGMRVHELLKYAYHHGVSTIVLENPEVIGRMRLAWIRNGKRLHRNYNWRVTIFRNRIIEMITMKAPIYGLRVNYVNPKGTTHSREHDDIMMRYGLDRHTASAYLIALKSI

SEQ ID NO: 2 - protein, 323 aa, synthetic construct
  REGION 1..323: note = Synthetic
VFRREAYSVIPSRRYADGVVALVRGIYESCRALGVSFRDAELGDWLMFQQSEREYPARNITLKAGYEFHVTVVGYDGSTGRVTVEPTVPKSYRVLLDGVLREKPKYCARVMVKGFGERKSDLWLNGEIHLTLPLELYHRCMAKDTPATGSNVAGVDVNVDRLNVAIVDTSGRLRDTTTFWFREVTSRGYPRRRARCVIGMSVHRMLKYLASHGVGMVVLENPEVIGYLRYCWVRSGERGSRNYNYKVAVFRSSIIEMIALKATLYGLKSVYVDPRGTSSSQEHRFVMEKHGLDRHTASAYLIALKHLQDEKIMNNRKAHEQHK

SEQ ID NO: 3 - protein, 290 aa, synthetic construct
  REGION 1..290: note = Synthetic
VLVRSIYESCRQLGVDFRSVELSDWLMFQQSELEYPAKSITLKPGYEFHITTVDYYKNTYRDVIKPIMSKSYRLLLEKVLEERQRYTGRVVIKSYGIRKDSLWIRGEVQITIPTDFYYKHMTRCREPRGSLICGVDVNIDRLNLAIIDEKGNLRDCKTFWFSETTARGFSRRSARSIISAKTREMLGYVHHHGVKTLLLENPEVLGKLKLLWVRSNGRRHENYNYRVSVFRSSVIEMIALKAPLYGIEVKYVDPKGTTNSREHDDAMRKYRLDRHVASAYLIALRGLKHQ

SEQ ID NO: 4 - protein, 368 aa, synthetic construct
  REGION 1..368: note = Synthetic
MNYVTLVLPFNIGGDTAKRLLSTAWLFKAAVHRVLGVAKQSPALPGTDIGWKSAFRSIAYSVIPNRRYADGAVTLVRSIYESCRQLGVDFRSVELSSWLMFQQSEKEYPARNITLKHGYEVHVTTISYSKESHRDVIKPSIPKSYSALLSRIIEERQEYSGRVVVKGYGVRGNTLWVRGEVQITIPADFYCKHMAKYREPKGSLIGGVDVNVDRVNLAIIDEKGSLRDYKTFWFTEATVRRYPRKRARSVIGMRVHELLKYAYHHGVSTIALESPEVLGRLKLLWARRGDRKHEDYNYRKSVFRSSVIEMIALKAPLYGMKVEYVDPRGTTNSTEHDRLMKVCGLDRHTASAYMIALRGLAHQVKQMK

SEQ ID NO: 5 - protein, 330 aa, synthetic construct
  REGION 1..330: note = Synthetic
MLSIAKQTSALPTTDIGWKDAFRKVIYEVMPNRSYVDGVIVLVRGIYESCRQLGVNFKEVELGNWLMFQQTEKEYPVRNITLKRGYEFYITTLDYDGNSERIVIKPTTLKNYKLLLDKILEESQKHTARVVVKDYGIRKGKLWVHGEIQLTIPLNFYYKYMTRYKRNYGKLYGGVDVNSDRINLAIVDKEGNLRDTYTFWFREVTARSYPRRKARTIIGMRIHEMLKYAYHHGVKTLFLENSEVLGRLKLMWMRNGKRLNRNYNWRVTTFRSSIIEMIAMKASLYAIRVMYVNPKGTTSSEKHDGIMRKYRLDKHTASAYLITLKGLDLL

SEQ ID NO: 6 - protein, 203 aa, synthetic construct
  REGION 1..203: note = Synthetic
VINELISGYYTYGAGVSIKVEKNEDWRDVWSKRLILYLNIPINLYVKYMGSVITNVSSNALLWAGFDFNVDRVCMAIVDSAGRLRDTKTVFFSNAVNMPREVSESMREEALTELVKYAVAHGVKYLAVEDLERPRRVKGKVGKWALRQYIQHLRVLAKRFNLDILEVNPAYSSIDAIGIALAQGLDKHTASAYLIALRGLRHQ

SEQ ID NO: 7 - protein, 167 aa, synthetic construct
  REGION 1..167: note = Synthetic
VELYIKHINPKNVDVAGTVNSNWLLAGFDFNVDRVCMAIIDPAGRLRDARTEFFSNAANTPKEVSENARKEAVVKLVKYAVAHGVRYIAVEGLRRPSKVEGRVGKWAHRQYLQHLKVLAKRFGLEVIEVNPAYSTIDAIGIALTLGLDKHTASAYIIALRGLAHKSM

SEQ ID NO: 8 - protein, 374 aa, synthetic construct
  REGION 1..374: note = Synthetic
MSLYQIAGRIIADNPRTLEDFKFVTINGKLVVDDPLDHMKLVKLARAYASAVRKLLNLIWGGHSHKEATKLLYNILPNYVYLETAYKNAKAITENIRFFEEYLGKNRILADIHRFWIASRGNKWDKGNRNIKLIPRDTYFEVLIKYPWDGSWIKARAFFGERYVPLLRELIELAGRREEGYGVVVSFREHPRIHIQIPLWLYLKYFSTSKPRGYGLIAGFDVNSDRLNVVVIDNDGNIVAVETFWYCETVSHGFPKGKAEWIRLNALSSALEWCRRIGADYVVFEDLTRIRCKRFTKNHYVNRKIAKFPKKQILIHGVIKALKLGFIVVLVNPRGTTNSVTHEQIMREKGLDRHMASAYMIAYRGLKVIRNHEK

SEQ ID NO: 9 - protein, 95 aa, synthetic construct
  REGION 1..95: note = Synthetic
MGLGFILISVSSSVRPPTPQLYVMVVTFISFIATIFWHGCYIVEHYVFKSRVISAGYDKYGNPRYSISIPKEVVEKIKRLHGREVIVHVIVPDSW

SEQ ID NO: 10 - protein, 364 aa, synthetic construct
  REGION 1..364: note = Synthetic
MMRSKLIASKEALDNFQFVTINGKVVFEDKDRIVRIARAYSQVVKSAIRPLFDGKSIDELTKEFYNILPNYVYLETALKQAKTITDGLLDREDEKGEIIHARIRKFWFASRGNKSDKGNRNVKFHVLEDHVEVKVKDPWNKEWIFGKAYFGKEYLPLLHELEELAERKEEGYGAVISFKHYPMIHLQVPLWLYLKHFSLPKPNGYGLVAGFDLNSDRLNVVVINKDGKVITTKTWWYSEVVSHGFPKEKARALRLNALSEALNFLSRIGVDYVVFEDLFLVRKKKFTRSKSGNRKVSRFAKKQMLIHGVIKALRLGFNVVLVNPKGTTNSEDHERVMREKGFDRHIASAYLIALKGLGMLNDNK

SEQ ID NO: 11 - protein, 71 aa, synthetic construct
  REGION 1..71: note = Synthetic
MIILTAKFINFAVTFYHMEQIVFRGVIASAGRDKYGDQRYTINVPKSVRDKVSKIVGKEVIVIVILPDDEE

SEQ ID NO: 12 - protein, 364 aa, synthetic construct
  REGION 1..364: note = Synthetic
MMRSKLIASKEAIDNFQFITINGKVIFNEKERVVRIARAYSQVIKSAIKPLFDGKSVDELTKEFYNVLPNYVYLETALKQAKTIVEGLLEREEERGEIIHSRIRKFWFGSRGNKSDKGNRNVKFHVLENYVEVRVKDPWNKEWIYGKAYFGKEYLPLLKELEDLSQRKEEGYGAVISFKEKPMIHLQVPLWLYLKHFSSLKPAGYGLIAGFDLNSDRLNVVAINKDGKVITTRTFWYSDVTRPGFPKEKARALRLNALSNALEFLSRIGVDYVVFEDLFLVKKRKFTRSKGGNRKISRFAKKQLLIHGVIKSLRLGFNVVLVNPKGTTNSEEHDRVMREKGFDKHTASAYLIALKGLGMLNDIK

SEQ ID NO: 13 - protein, 54 aa, synthetic construct
  REGION 1..54: note = Synthetic
VEQIVFRSTVSSYGTDKYGNKRYGITIPSKLRDKGEKLYGKEVIVIVILPDDEE

SEQ ID NO: 14 - protein, 363 aa, synthetic construct
  REGION 1..363: note = Synthetic
MMKSKLIGSMEALDNFQFVTINGKVVFEDKDKVVRIARAYSQVVKSAIRPLFDGKSVDELTKEFYNILPNYIYLETALKQAKTITDGLLDREDEKGEIIHARIRRFWFGSRGNKSDKGNRNIKFHVLEDHVEIKVKDPWGKWVHGKAYFGKEYLPLLKELEELSKRKEEGYGAIVSFKHYPMIHLQIPLWLYLKYFSSPKPNGYGLIAGFDLNSDRLNVVVINKDGKVVAKKTWWYSEVVSHGFPKEKARALRLNALSESLNSLSRIGVDYVVFEDLFLVKKRKFTRSKSGNRKVSHFAKKQLLIHGVIKALRLGFNVILVNPKGTTNSEDHERVMREKGFDRHIASAYLIVLKCLGMLNDAK

SEQ ID NO: 15 - protein, 362 aa, synthetic construct
  REGION 1..362: note = Synthetic
MRNKLIGSREALDNFQFVTINGRVLFNEKERVVRIARAYSQVVKSAIKPLFDGKSIDELTKEFYNVLPNYIYLETALKQAKTIVDGLLEREEEKDKILHAKIRKFWFASRGNKSDKGNRNVKFHVFQDHVEVKVKDPWGKWVYGKAYFGKEYLSLLHELEELASNKEEGYGAVISFEEKPMIHLQIPLWLYLKHFSSPKPVGYGLIAGFDLNSDRLNVVVVNEEGKVVTKKTWWYSDVTRPGFPKGKAMALRLNALSQALNFLSRIGVDYVVFEDLFLVKKRKFTRSKSGNRKITRFAKKQLLIHGVIKALRLGFNVILVNPKGTTNSEDHEKVMREKGFDRHTASAYLIALKGLGMLNDIK

SEQ ID NO: 16 - protein, 54 aa, synthetic construct
  REGION 1..54: note = Synthetic
MEQIVFRSTVSSYGTDKYGNKRYGITIPSKLRDKGEKLYGKEVIVIVILPDDEE

SEQ ID NO: 17 - protein, 361 aa, synthetic construct
  REGION 1..361: note = Synthetic
MTNKLIGSKEALEHFQFVTINGKVNFEDKERVVRIAKAYSQVVKSTIRPLFDGKSIDELTKEFYNLLPNYLETALKQAKTIIDGLLEREENRGEMIHARIRKFWFGSRGNKADKGNRNVKFHVLEDHVLIKVRDPWNKEWIFGKAYFGKEYLPLLKELEDLSQRKEEGYGAVISFKEKPMIHLQVPVWLYLKHFSSLKPAGYGLIAGFDLNSDRLNVVVINKDGKVITTRTFWYSDVTRPGFPKVKARALRLNALSNALEFLSRIGVDYVVFEDLFLVKKRKFTRSKSGNRKITRFAKKQLLTHGVIKSLRLGFNVVLVNPKGTTNSEEHDRAMREKGFDRHIASAYLIALKGLGMLNDIK

SEQ ID NO: 18 - protein, 53 aa, synthetic construct
  REGION 1..53: note = Synthetic
MEQIVFKSTVISYGTDKYGNKRYGITIPAKIREKGEKLYGKEVIVIVILPDDE

SEQ ID NO: 19 - protein, 360 aa, synthetic construct
  REGION 1..360: note = Synthetic
MRNRIICSREALENFQFVTINGKVIFEDKDKIVRIARAYSQVVKSAIRPLFDGKSIDELTKEFYNILPNYIYLETALKQAKTIVDGLLDREKDKILHAKIKRFWFGSRGNKSDKGNRNVKFHVFQDHVEVKVKDPWGKWVHGKAYFGKEYLPLLHELEELAERKEEGYGAVVSFKHYPMIHLQIPLWLYLKHFSSPKPNGYGLVAGFDLNSDRLNVVVVNKDGKVVTKKTWWYSDVTRPGFPKGKAMALRLNALSQALNFLSRVGVDYVAFEDLFLVKKRRFTRSRSGNRKVSRFAKKQLLTHGVVKALRLGFNVVLVNPKGTTNSEDHDRIMREKGFDRHTASAYLIALKGLGMLNDIK

SEQ ID NO: 20 - protein, 54 aa, synthetic construct
  REGION 1..54: note = Synthetic
MVQVTFHSKIFSMGHDKYGDPKYAIYVPKSIHEKIKGLLEKEVIVIVILPDDEE

SEQ ID NO: 21 - protein, 360 aa, synthetic construct
  REGION 1..360: note = Synthetic
MPRIIADKETLEQFQFVTINGRLIPKTGEDFMKLCFLAYRFRESVRHELNLVFRDVPQRDAYKELAKMLPSSIYGETAYKYAKLLAEGSKGKIKVRRIWVASRGGVTWGGNLNIRLASSNRVLVRYYGGEWIELESRFGKKYIPLIEELIKLAKGRKESYGVVISFRNGKMYIHLEVPLHLYLKHFSEPKQRGYNLVAGFDVNSDRVNVAVLDINASIVALRTFWYSEVTSHGFPAERAKWIRLNALSNALKWCRSIGVDFVVFEDLTKVKKRRFTGNSNANRKISKFAKKQIIRHGVLKALKIGFTVILVNPEKTTNSLTHKQIMKEKGLDRHMASAYITAYRGIKKIKEQKTATTPPT

SEQ ID NO: 22 - protein, 56 aa, synthetic construct
  REGION 1..56: note = Synthetic
VLMARIMSFRARITRHGTARGRPRYAVLIPMELSEKVEKEGWIHREVEVIIVEPDA

SEQ ID NO: 23 - protein, 359 aa, synthetic construct
  REGION 1..359: note = Synthetic
MMRSELTGSKEALGKFQFVTISGRVEFEDVGRVVKIAHYYSRAVKAGINLALKGVPLNDAVKELYRIIPYAFYAETAYKQALALIKNGGNKIEIRRRWIACKGSKADRGNRGIKFHVLEDHVEVKVKDPWGRWIVGRAYFGRKYLPLLRELEELAGKGEEGYGAVISFKEKPMIHLQIPLWLYLKHFSVKKPIGYGLIAGFDLNSDRLNVVVIDRDGRIVTTRTYWYPEVTRPGFPREKGKALRLNALSNALKFLSRIGVDYVVFEDLFLVKGRRRFTKSKSGNRRISRFAKRQLLTHGVIKSLRLGFNVILANPKGTTNSKEHEKVMKERGFDRHTASAYLIALRGLETNSIKGVRSN

SEQ ID NO: 24 - protein, 354 aa, synthetic construct
  REGION 1..354: note = Synthetic
MMRSKLIGSKEASDNFQFVTINGRVEFEDVERISRIAYYYSKAVKAGINLALKGVSLNDAVKELYKIIPYAFYAETAYKQALALVENKGSKVEIKKRWIACRGNKSDKGNRGIKFHVLEDHILIKVKDPWGKWVHGKAYFGKKYLPLLSELEELSRRKEEGYGAVISFKHYPMIHLQVPLWLYLKHFSSPKPNGYGLFAGFDLNSDRLNVVVVNEEGKVVTNKTWRYSDVTRPGFPKGKAMALRLNALSESLNFLSRVGVDYVVFEDLFLVKRRKFTRSKSGNRKIMRFAKKQLLIHGVIKALRLGFNVILVNPKGTTNSEYHDRIMREKGFDRHTASAYLIALKGLEVIKNNE

SEQ ID NO: 25 - protein, 54 aa, synthetic construct
  REGION 1..54: note = Synthetic
MEQIVFRGVISSAGSDKYGEKRYAIYIPKSVKEKAGKIAGNEVVVIVILPDDEE

SEQ ID NO: 26 - protein, 354 aa, synthetic construct
  REGION 1..354: note = Synthetic
MMGSRLIGSKEALENFQFVTINGRVEFEDIGRISRIAYYYSKAVKAGINLALRGVPLDDAVKELYKIIPYAFYAETAYKQALALVKNNGNKVEVRRRWIACRGNKSDKGNRGIKFHVSEDHVEVKVKDPWGTWIVGRAYFGRKYLPLLRESEDLASRREEGYGAVISFKERPMIHLQIPLWLYLRYLSSPKPPGYGLVAGFDLNSDRLNVVVINRDGGVITTKTFWYSDVTRPGFPKERAKALRLNALSEALNFLSRIGVDYVVFEDLFLVKRRKFTRSKSGNRKVSRFAKKQLLTHGVIKALRLGFNVVLVNPKGTTNSEEHEKVMREKGYDRHTASAYLIALKGLEVIKNNE

SEQ ID NO: 27 - protein, 54 aa, synthetic construct
  REGION 1..54: note = Synthetic
MEQIVFRGVISSAGSDKYGEKRYAIYIPKSVREKAGKIAGKEVIVIVILPDDGE

SEQ ID NO: 28 - protein, 366 aa, synthetic construct
  REGION 1..366: note = Synthetic
LSSYQMMRNKLIGSREALDNFQFVTINGRVLFDEKERVIRIARAYSQVVKSAIKPLFDGKSVDELTKEFYNILPNYVYLETALKQAKTIVDGLLDREKDKIIHAKVKRFWFGSRGNRSDKGNRNVKFHVLKDHVEIKVKDPWGKWVYGKAYFGKQYLSLLHELEELASNKEEGYGAVISFEEKPMIHLQIPLWLYLKYFSSPKPNGYGLIAGFDLNSDRLNVVVINKDGEITANKTWWYSDVTRPGFPKGKARALRLNALSESLNSLSRIGVDYVVFEDLFLVKKKKFTRSRSGNRKVSRFAKRQLLIHGVIKALRLGFNVVLVNPKGTTNSEDHDRIMREKGFDRHIASAYLIALKGLGMLNDAN

SEQ ID NO: 29 - protein, 54 aa, synthetic construct; identical to SEQ ID NO: 16
  REGION 1..54: note = Synthetic

SEQ ID NO: 30 - protein, 374 aa, synthetic construct
  REGION 1..374: note = Synthetic
MPKIIADKETQEQFMYITINGRLIPKSNENYMKLCLLAYRFKQAVKHGINLVLSDVPQKQAYKELAKILPSSIYGETAYKYAKLLVEGAREQVNNEVIVNRISTKRKWIASRGGVTWRGNLNIKLISTDKVLVRYYDGEWLEFKVRFGEKYLPLVNELIELANQKRTSYGASISFKYGKPYIHVELPLWLYLKHLLAPKPNGYGLIAGFDINSDRLNAVIIDRECEIIALRTFWYSEAISHGFPRNKAKWIRLNALAKALKWCRRIGVDYVVFENLTKIKTRRFTSIPNANRKIAKFPKKQLLKNSIIKALKLRFTVILVNPRGTSSSIAHKQIMREKGLDKHATSAYIIAYRGLKTLKEQEPSITPSTTSTIN

SEQ ID NO: 31 - protein, 47 aa, synthetic construct
  REGION 1..47: note = Synthetic
VEQYVFHAKISKCSGRKLCIYIPREIEDRLKHLHGQEVVVVVIKPDA

SEQ ID NO: 32 - protein, 374 aa, synthetic construct
  REGION 1..374: note = Synthetic
MPSIIADRETIEQFMYVTINGRLVPKSNEDYMKLCLLAHRFKQAVKHGVNLVLEKIPQKEAYKELAKLLPSSIYGETAYKYAKLLVKGAKEQVSNEVIVNRINIKKKWVASRGGVTWRGNLNIKLISTDRVLVRYYNGEWLEFKMRFGEKYLSLINELIELASYKRISYGITISFRKGRSYIHVEVPLWLYLKHFSSPKPKGYGLIAGFDINSDRLNVVVLSGDGSIAVMKTSWYSETVSHGLPKNKAKWLRLSALSEVLKWCRRIGVDYIVYEDLTKIKTRRFTSNPCANRKITRFPKKRFLKHGVIKALKLGFTVILVNPRGTSSSITHKQIMIEKGLDKHMTSAYIIAYRGLKKLKEQKPSITPSITPIIN

SEQ ID NO: 33 - protein, 54 aa, synthetic construct
  REGION 1..54: note = Synthetic
MGRILSFRARISRHGSVKNKPRYAILIPMELSEKIKKEGWIGREIEVIIIEPDA

SEQ ID NO: 34 - protein, 374 aa, synthetic construct
  REGION 1..374: note = Synthetic
MPKIIADKETAEQFMYATVNGRLIPRSNEDYMKLCLLAYRFKQAVKHGVNLALRRVPQKEAYKELAKILPSSIYGETAYKYANLLVESAKEQIDNEIYINKITIKKKWIASRGGVTWRGNLNIKLTSTNKVLVRYYNSEWLEFKVAFGERYLSLIKELVKLAKQKKISYGVTISFRNGKLYIHIEIPIWLYLKYFSQPKSKGYGLIAGFDLNSDRLNVIVINGNGDIVASKTCWYSEVVSHGFPRNKARWRRLNVLSNILKWCKRIGVDYVVFEDLTKIKTRKFTHNPNINRKITKFPKKQLLRHGVIKALKLGFTVILVNPKGTSGSVTHRQIMREKGLDRHMASAYMIAYRGLKKLKEQDPSITPSTTPTIN

SEQ ID NO: 35 - protein, 53 aa, synthetic construct
  REGION 1..53: note = Synthetic
LLWCTAVEQYVFHAKISKGSGKKLCIYIPKEIEDKMRRLHGQKVIVIVIKPDA

SEQ ID NO: 36 - protein, 374 aa, synthetic construct
  REGION 1..374: note = Synthetic
MPKITADKETLERFTYITVNGKLIPRSNEDYMKLCLLAYRFKQAVKHGINLVLKNTPQGKAYKELAKLLPSSIYGETAYKYANLLVESAKEQIDNEIYINKIVIKKKWIASRGGVTWRGNLNIKLTSTNEVLVRYYNGEWLEFKVAFGERYHSLIKELVKLANQKKISYGVTISFRNGKPFIHVEIPIWLYLKYFSQPKLKGYGLVAGFDLNSDRLNVVVINGNGDIVALKTFWYSEVISHGFPRNKARWRRLNVLSNMLKWCRRVGVDYVVFEDLTRIKARKFTHNPNINRKIAKFSKKQLLRHGVIKALKLGFTVILVNPKGTSSSVTHRQIMREKGLDRHMASAYMITYRGLKKLKEQEPPITPSTTAGYP

SEQ ID NO: 37 - protein, 53 aa, synthetic construct
  REGION 1..53: note = Synthetic
LLWCTAVEQYVFHAKISKGSGRKLCIYIPKEVEDKIRHLHGREVIIVVIKPDA

SEQ ID NO: 38 - protein, 354 aa, synthetic construct
  REGION 1..354: note = Synthetic
MMRSKLIGSKEAIENFQFVTINGRVEFEDVEKISRIAYYYSKAVKAGINLALRRVSLNDAVKELYKIIPYAFYAETAYKQALALVENNGNKVEIRKRWIACRGNRSDNGNRGIKFHVLENHVLIKVKDPWGKWVYGEAYFGKEYLPLLKELEELSRRKAEGYGAVISFKHYPVIHLQIPLWLYLKHFSSPKPNGYGLFAGFDLNSDRLNVVVVNKEGKVVTSKTWRYSDVARPGFPKDKAKALRSNALSQALNFLSRIGIDYVVFEDLFLVKRREFTRSKSGNRKASRFAKRQMLTHGVVKALRLGFNVILVNPKGTTNSEAHDRMMREKGFDRHTASAYLIALKGLEVIRNNE

SEQ ID NO: 39 - protein, 57 aa, synthetic construct
  REGION 1..57: note = Synthetic
MLTEIFINISVTNNMWFKLHFAQRSPQWVTISTVTRNTRYTSLRAFTRRSRACWERR

SEQ ID NO: 40 - protein, 362 aa, synthetic construct
  REGION 1..362: note = Synthetic
MKSRIIGSKEALDNFQFVTINGRVLFNEKERVVRIARAYSQVVKSAIKPLFDGKSVDELTKEFYNILPNYIYLETALKQARTITDGLLDREDEKGKIRHARIRKFWFGSRGNKSDKGNRNIKFHVLEYHVLIKVKDPWGKWVYGKAYFGKEYLPLLHELEELAERKEEGYGAVISFKHYPMIHLQIPLWLYLKYFSSLKPNGYGLVAGFDLNSDRLNVVVVNKEGSIVTTKTWWYSEVVSHGFPKGKAMALRLNALSQALNFLSRVGVDYVVFEDLFLVKNRRFTKSKSGNRKITRFAKRQMLIHGVIKALRLGFNVILVNPKGTTNSEEHDRVMREKGFDRHTASAYLIALKGLGMLNDIK

SEQ ID NO: 41 - protein, 71 aa, synthetic construct
  REGION 1..71: note = Synthetic
MLILTEKFINFAVTYNHMEQIVFRGVISSAGRDKYGDQRYTINVPKSVRDKVSKIAGKEVIIIVILPDDEE

SEQ ID NO: 42 - protein, 362 aa, synthetic construct
  REGION 1..362: note = Synthetic
LMPRIIADEETIEQFMYVTVNGRLKPKSSEDYMKLCLLAYRFKQTVKHGLDLVLKGIPQKEAYKELAKLLPSSIYGETAYKYAKLLVEGSNERKIKIRRIWVASRGGVTWRGNLNIRLVSTNRVLIRYYNGEWLEFSARFGRKYLPLIRELIELAKYKRISYGVSISFNNGKPYVHVEIPLWLYLKHFSTPKSEGYGLIAGFDLNSDRVNVVVINGEGDIVALKTFWYSDVVSHGFPREKARWLRLNALADALKWCRRIGVDYIVFEDLIKIKTRKFTSNPTINRKITRFPKKQMIRHGVMKALKLGFTVILVDPRGTSNSITHRQIMIEKGLDRHMASAHMIAYRGLKRLKEHIPSTTPPT

SEQ ID NO: 43 - protein, 56 aa, synthetic construct
  REGION 1..56: note = Synthetic
VVMAKFLCFRARISRHGSVKGRPRYAILIPMELSEKLEKERWIGREVEVIVIETDA

SEQ ID NO: 44 - protein, 374 aa, synthetic construct
  REGION 1..374: note = Synthetic
MSSYQIAGKVITDNPKALESYRFVTINGKLVLDNPLDHMKLVRLARAYSIVVRKSLNLIWGGHSHKEATKLLYNILPNYVYLETAFKNAKAIAENIRFYEKNLGKDRVLAHIRRFWVASRGNKWDRGNRNIKLIPRDTHFEVLIKYPWDSSWIKAKAFFGKRFIPLLRELTQLANEKKEGYGIVVSFREHPRIHVQVPLWLYLKYFSTPKQEGYGLIAGFDINSDRVNVVVLDGNGSIVAMKTFWYSETVSHGFPRDRAKWVRLNALSDALGWCRRIGVDYIVFEELTKIKIKKYVSNPSINRKISKFPKKQVLVHGVIKALKLGLTVVLVNPRGTSNSVTHRQIMREKGLDRHMASAYIIAYRGLKVIRNNQK

SEQ ID NO: 45 - protein, 72 aa, synthetic construct
  REGION 1..72: note = Synthetic
MVVTFISFTATTYYRGCHTMEHYVFRSRVISAGYDKYGNPRYSISIPKEIVEKIKHLHGREVIVHVIVPDSW

SEQ ID NO: 46 - protein, 380 aa, synthetic construct
  REGION 1..380: note = Synthetic
LSLSLIPKIIADKETLEQFMYVTVNGKLIPRGNEDYMKLCLLAYRFKQAVKHGINLVLKSTPQREAYKELARLLPTSIYGETAYKYAKLLVEGAKEHLDNGNKVINRICIKRKWLASRGGVTWRGNLNIKLLSTDKVLIRYYDNEWLEFNIRFGSRYVSLIKELIELANQKRISYGVSISFNNGKLYIHIEIPLWLCLKRFSMSKPRGYGLVAGFDFNSDRLNVVVIDGEGNIVAIKTFWYSEIISHGFPKGKARWLRLNVLANALKWCRKIGVDYIVFEDLTRVKNKRFTSNPYANRKITKFPKKQMLRHGVIKALKLGFMVILANPRGTSNSITHKQIMRERGLDRHMASAYIIAYRGLKKLKEQEPSTTPSTTPIIN

SEQ ID NO: 47 - protein, 47 aa, synthetic construct
  REGION 1..47: note = Synthetic
VEQYVFHAKISKGSGKKLCIYIPKEVENKIRHLHGQEVIVIVIKPDT

SEQ ID NO: 48 - protein, 368 aa, synthetic construct
  REGION 1..368: note = Synthetic
LLSYQMMRNRVIGSKEALENFQFVTINGRVLFNEKERVVRIARAYSQVVKSAIRPLFDGKSIDELTKEFYNILPNYVYLETALKQAKTIVDGLLDREDEKGEIIHAKVKRFWFASRGNKSDKGNRNIKFHVLEDHVLIKVKDPWGKWIYGKAYFGKEYLPLLKELEELSRRKEEGYGAVISFKDHPMIHLQIPLWSYLKHFSSPKPKGYGLIAGFDLNSDRLNVVVINKESSIVTAKTWWYSEVVSHGFPKDKAKALRLNALSQALNFLSRIGVDYVVFEDLFLIKNRRFTRSKSGNRKVSRFAKKQLLIHGVVKSLRLGFSVVLVNPKGTTNSEDHNRIMRKKGFDRHTASAYLIALKGLGMLNDIK

SEQ ID NO: 49 - protein, 42 aa, synthetic construct
  REGION 1..42: note = Synthetic
MGHDKYGDPKYAIYVPKSVHEKIKGLLGKEVIVIVILPDDEE

SEQ ID NO: 50 - protein, 368 aa, synthetic construct
  REGION 1..368: note = Synthetic
LLSYQMMRSKLIGSKGAIENFQFVTVNGKVIFEDKDKIVRIARAYSQVVKSAIKPLFDGKSVDELTKEFYNILPNYIYLETALKQAKTIVDGLLEREDEKGEIIHARIRRFWFGSRGNKSDKGNRNIKFHVLEDHVLIKVKDPWGKWVFGKAYFGKEYLPLLKELEDLASKKVEGYGAVISFEEKPMIHLQIPLWLYLKHFSSPKPNGYGLIAGFDLNSDRLNVVVVSKDGKVIANKTWRYSDVVRPGFPKGKAMALRLNALSQSLIFLSRIGVDYVVFEDLFLVKRRKFTRSRSGNRKVSRFAKRQMLTHGVIKALRLGFNVILVNPKGTTNSEEHEKVMREKGFDRHTASAYLIALKGLGMLNDIK

SEQ ID NO: 51 - protein, 54 aa, synthetic construct; identical to SEQ ID NO: 20
  REGION 1..54: note = Synthetic

SEQ ID NO: 52 - protein, 269 aa, synthetic construct
  REGION 1..269: note = Synthetic
LRITVTRSRLERGGSLVGGNKSDNGNRGIKFHVFQDHVEIKVKDPWGKWVHGKAYFGKKYLPLLKELEELSRRKEEGYGAIVSFKDYPMIHLQIPLWLYLKHFSSPKPNGYGLFAGFDLNSDRLNVVVISKDGEITANKTWWYSDVTRPGFPKGKAMALRLNALSESLNFLSRVGVDYVVFEDLFLVKRRRFTRGKSGNRKITRFAKRQMLTHGVIKALRLGFNVVLVNPKGTTNSEDHDKVMREKGFDRHTASAYLITLKGLEVIKNN

SEQ ID NO: 53 - protein, 54 aa, synthetic construct
  REGION 1..54: note = Synthetic
MVQVTFHSKISSMGHDKYGDPKYAIYVPKSVHEKIKGLLEKEVIVIVILPDDEE

SEQ ID NO: 54 - protein, 345 aa, synthetic construct
  REGION 1..345: note = Synthetic
VAGEGEEIRATVLGKLDFENGENRALLLYTMRLFRDAVEFAHNLLKKGLSKNQIVKLVTSRVLNNKWYSMSAYVRAKLYEEQQFLKLRKPQLYSVGSSDEGGNRNIKFNSTNSVKIKIPSADGRHRWIWCKVKFGRKHIPVVRRVVEESISYGAGIAIKDGKFFLYMNIPVEIYAREMRKEIKSKVEPKHLAGFDFNTDRINMVIVNEKGEIRDVKNEHFPEITSHGFPKEKAKTARQRALAELVKYAREHGVRYFVVERLEKTGTKTKSKTVNRKIAKFALREYLNQMEILVEKVGGRLIEVDPAYTSIDAMPLAKKLGIDRHTASAYLIALRGLILLKNHKNA

SEQ ID NO: 55 - protein, 59 aa, synthetic construct
  REGION 1..59: note = Synthetic
MGKKRKVMLTVDEELYREFKLYALRKFSDVRNFVSKALEEAMSLWLEREKKLERPFSGN

SEQ ID NO: 56 - protein, 339 aa, synthetic construct
  REGION 1..339: note = Synthetic
VGKEVRPTIKGKLVLSSAEDKAFLLYVMRNFKDAVDYAHSLLKKGLKENKIIKLLTSRILNNKWYSRSAYIRAKLYSEQPYLKLKKPQLYSVGVKAEKGNRNIRLMSTEIVLIKIPSATGKHRWLKAKVKFGRKHIPIIQELTNSNISYGAGICTKDSKLILYINIPLELYVKYMRKESKTKITEHIAGFDFNPDRINMVIIDRQGIMRDVRNEHFMEITSHGFPREKAVSLRRESLTKLVKYAREHGVKYYVIERLSKPKPKGNKTAKRKISKMALREFTQQMEVLVPKVEGELIKVNPAYSSISAKIIAGDLGLDVHTTSAYIVAMRGLKKIQKAAV

SEQ ID NO: 57 - protein, 52 aa, synthetic construct
  REGION 1..52: note = Synthetic
MELIGVYRVLPKMKMTIPKEIAEKWDLKPKDKLVVYYDEKNGRMVVEKWEKK

SEQ ID NO: 58 - protein, 333 aa, synthetic construct
  REGION 1..333: note = Synthetic
VTIRGRLILNSIVDKAWLLLLMRNFRDAVEYAHNLLRKNVSENDIVKLLASRILSNAHYSYSALQRAKMYRQQPYLKLKKPQLFSIGKRNEKGNRNIRFLSTDRVQIKIPSATGKHRWITAGVRFGRKYISTIKKLINNSIPYGAGVYINSGLELHVNVPLELYTEKLKVGNKNTKNDKGYIASFDFNSDRICMVIVSREGRILDVKNKHFPEVTSPGYSGRKARDKRMKALAELVDYAYHHNVTDYVAEILSRPKKKSRSKTGNRKSSRWALREFVTHLKTLVARYGGKPHFINPAYTSVDAIPLSKQLGLDIHTTSAYLLAIRFIKGYEEL

SEQ ID NO: 59 - protein, 47 aa, synthetic construct
  REGION 1..47: note = Synthetic
VYRVLPKMRMTIPKEVAEKMGLKVGDKLVVYFDNKKQRMVVEKWRKG

SEQ ID NO: 60 - protein, 349 aa, synthetic construct
  REGION 1..349: note = Synthetic
MAGKREEVRATIKGKLAFDSDFDRALLFYTMRLFKDAVEMAHNLIRKQLKKNEIVKLLTSRILNNKWYSVSAYMRAKLYENQPHLKLRKPLLYSLGSSDENGNRNIKLIKTSLVKIKIPSADGKHKWIECKVKFSKKHLPVIQDLIEEKTSYSVGISLNDMNFILYINVPVEVYVDKAKKTIKSKSIPKHFVGFDFNPDRINMVIIDENGEIRDIKNAHFHEVISHGFSKDKAKQIRQEFLVKLVKYAREHGVRYFVVEQLKKPEPRGSKKAKRKISKMALMEYLNQMKILVRKVGGKLIKVNPAYTSIDAIPISKKLGIDIHTASAYLIALRGLRLIKTHKIPKSYLF

SEQ ID NO: 61 - protein, 55 aa, synthetic construct
  REGION 1..55: note = Synthetic
MSKKRKVMLTINEELYGEFKVYALKKFKDVRNFVSKALEEALRLWLEKEKKSERR

SEQ ID NO: 62 - protein, 342 aa, synthetic construct
  REGION 1..342: note = Synthetic
VAKRGKGKATVIGKLTVDDPVNKAKLLHLMRMFRDAVEMAHYHMRKKFEASESKRRLTKFLCNAWYASSAMKKAKLYVSQEKLKLTKPQLFSVGCSGNMEKGNRNIRLISTDKVLVKIPHADGKHEWLELKTKFGKKYLDLVSKLISEEYSYGASIVLKEENGKINFFLHISIPIDLYVQFFKKSSRNKIVGYIAGFDFNSDRINMVIINKNGILLDVKSEHFPEVTSHGFPREKAKGLRRQALAKLVKYARNHGVKYYVVENLERSKKKTICRKANRKISKWAVREYLTQMEILVKKVDGILVKINPMYSSVAARFLAKDLGLDVHTTSAYIIALRSLKLL

SEQ ID NO: 63 - protein, 50 aa, synthetic construct
  REGION 1..50: note = Synthetic
MVRKTIDIPDRLWREFEIEAVKKFGHYGAIKKAIREAIELWLKEVKEKQQ

SEQ ID NO: 64 - protein, 346 aa, synthetic construct
  REGION 1..346: note = Synthetic
MVRTGEEGRATIVGKIEFQNETDKALLLYTMRLFKDAVEFAHNLFRKDLPKNEIVKLLTSRILHNKWYSVSAYIRAKLYENQSYLKLKKPQLYSAGSSDENGNRNIKFVRTDLVKIKIPSADGKHRWIECKVKFGRKHIPIIEELLRGGQTYGAGVSLSDDGKYVLYVNVPRKLYVERMKKKIKTNAKARLFAGFDFNPDRINMVIVDKKGQIREARNEHFHEVTSHGFPREKAKAIRREALARLVKYAKEHGVKYFVVERLEKPKTKTKSKSANRKITKFAVREYLEQMRILVEKVGGKIIEVDPAYTSIDAIPLAKKLGIDRHTASAYLIALRGLMLLKNHKNT

SEQ ID NO: 65 - protein, 59 aa, synthetic construct
  REGION 1..59: note = Synthetic
MGKRRKVMLLIDSDLYREFQVYALKKFYGRRDYVSAAFEEAVRLWLEQEKRAEQQSSGK

SEQ ID NO: 66 - protein, 335 aa, synthetic construct
  REGION 1..335: note = Synthetic
MKPTIKGKLLFDSLEDRALLLYAMRNFKDAVEYAHSLMRKGVRDGEIVKLLTSRILNNKWYSVSALKRAKLYRDQPYLKLRRPQLFSVGSRDEGGNRNIKLDATDTVRIKIPSASGRHRWITAKAQFSKKHLPIIQELVNVRTSYGAGIYLKNGRFEIHVNVPLELYVKHMRKKTRAKVADHFAGFDFNPDRICMVIIDRKGIIRDIKSERFPEVTSHGYPRDKAGAVRQEALAKLVKYARDHGARYYVIERLSRPKPKGSKSARRKISKMALRQFTQQMEVLVPKVGGVLVKVNPAYSSVSAKIVAEDLGLDVHMASAYIIALRGMKKIQKDTE

SEQ ID NO: 67 - protein, 52 aa, synthetic construct
  REGION 1..52: note = Synthetic
MELIGVYRVLPKMRMTIPKEVAERMGLREGDKLVVYYDAENGRMVVEKWRKK

SEQ ID NO: 68 - protein, 345 aa, synthetic construct
  REGION 1..345: note = Synthetic
MVRGGEEIRATVLGKLDFENGENRALLLYTMRLFRDAVEFAHNLLKKGLSKNQIVKLVTSRVLNNKWYSMSAYVRAKLYEEQQFLKLRKPQLYSVGSSDEGGNRNIKLVSSSIVEIKNPSANGRHRWISCKVGFGRKHIPVIRRVIGEGISYGAGISLKDGKFFLYVNVPVEIYAEEMRNEIRSNAKPKYFAGFDLNSDRINMVIVDGRGEIRDVKSEHFPEVTSHGFPKEKAKTIRQHALAKLVKYAREHGAKYFVIEILKRTKTKTMSKTANRKISKFALREYLNQMRILAKKIDGTLIEVDPAYTSIDAIPLAKKLGIDRHTASAYLIALRGLMLLKNHKNI

SEQ ID NO: 69 - protein, 59 aa, synthetic construct
  REGION 1..59: note = Synthetic
MGNKRKVMLTVDEELYREFKLYALRKFSDVRNFVSKALEEAMRLWLEGEKKLERPSSGN

SEQ ID NO: 70 - protein, 355 aa, synthetic construct
  REGION 1..355: note = Synthetic
MEKVKPTIRGKLVFDSAEDKAFLLYAMRNFKDAVEYAHSLMRKGLKRNKIERILTSRILKNKWYSISAFKKARLYRDKPYLKLRRPQLFSVGGENENGNRNIRFDSTNMVRIKIPSTTGRHRWITAKTQFGKEQIPIVNELVNPSFPYSAGVSLKNNEFRLYVNVPLELYVEHMRKKSDAKIVGYIAGFDFNPDRINMVIIDSQGIIRDIQNEDFPEVTSPGFPKEKSETIRKEKLAKLVKYARNHGVKYYAVEKLSKPKPEGCRNAKRKISKMALKQFIQQIEVLVPKVNGVLVKVPPAYSSVSAKIIAEDLGIDIHTASAYIIALRGYKKIQKNKKQATADKLRPQEFCFLKN

SEQ ID NO: 71 - protein, 80 aa, synthetic construct
  REGION 1..80: note = Synthetic
MLIPFVILIGVGLTKEFMWLGYINVLGDTMELMGVYRVLPKVRMTIPKEVAERMGLKVGDKLIVYYDEENQRMVVEKWKK

SEQ ID NO: 72 - protein, 352 aa, synthetic construct
  REGION 1..352: note = Synthetic
MAKRTKEEKVTIKGKLVIHDEIVLSKLINLMRQFRDAVELAHYLLFKRKLKDSEVKRRLTRLLSNAWYGYSVIKKAKLYQKQPHIKLRKPLLFSVGCKGLEKGNRNIRLVDTDKVLIKVPHANGKHEWIECKVEFGRKYLELVKELVLGEYPYSATITVKLKNKEDWRKVFRKRLYLHLIIPLDLYLKYFSRKPKNNIVGHVAGFDFNVDRVNMVIIDQKGIIRDIKNEHFPEVTSHGFPRNKAKATRRESVAKLVKYAREHGVKYYVIEDLSKPKPKGTRTAKKKIYRMALREFIQQMEVLVPKVDGVLIKVNPAYSSVSARIIAEDLGLDVHTASAYIIAKRGLTNLQKP

SEQ ID NO: 73 - protein, 62 aa, synthetic construct
  REGION 1..62: note = Synthetic
LPLPPYGSSYALYSELPSGNRGSSGIHQPQDLIGFKRTTFIDSMKSFISLTFHRCLLGMGFG

SEQ ID NO: 74 - protein, 345 aa, synthetic construct
  REGION 1..345: note = Synthetic
MARKREEVKPTIVGRLYLESPVDKAKLLHLMRKFRDAIEMAHYHMRQGRDINEIHRRLTRFLDNGHYAHSAYQRARLYKNQPYLKLRKPQLFSVGRACEKGNRNIRLSSTERVKIKIPHADGKHEWIEGRTRFGSRYLPLIEELVKLENISYSAGIYIEDRGFVICISVPLELYVKHFSKKPRNKIVGHIASFDLNPDRICMVIVNQQGKILDIRNEYFPEVTSHGFPREKAMHLRREAISKLVTYASEHGVEHYVVERLSKPRGKAKSKSGRRKQSKFAVREYLQQMQVLVPKVGGKLHKLNPAYVSIDAIPLSKKLGLDIHTTSAYLLAIRYIRNRRTKRNKN

SEQ ID NO: 75 - protein, 51 aa, synthetic construct
  REGION 1..51: note = Synthetic
MVKKTIEIPDDLWEEFMFKAVKKFGYYGAIKKAVKEAVELWLEREKKLSQQ

SEQ ID NO: 76 - protein, 376 aa, synthetic construct
  REGION 1..376: note = Synthetic
LNRPVTVDIPFTVTGEKARLVWWTAWFSKFCAHRLLNDAKENEILVDLSQTSFLKYARKRCYDVLPNRRYVDGIAVLIHSTLKSARKLGLKVNEIKLRQWLLFQCEADKKKKGNLNVRLTSTREAEALVFNQEREPRKIKINLRTPKKYEKLLKILVERAIKSEVGYPARIVIKEYNTHLNDLRLYCSLQVMVPYDLYLEVMRKYDAPLGNHVAGIDVNVDRLNIAIVDRYGRLRDVKTFWFREITSRGYRRKRAWTKIYQAIHDMLNYAYHHGVSHVVLENPEIVGYLKYYWIKNGNRKTKNYNYRTSIFRNRIIETIAYKTPLYGLKTIYVNPKGTTRSKEHEKIMKKYGLDRHTASAYLIALKSLKKIHYATK

SEQ ID NO: 77 - protein, 372 aa, synthetic construct
  REGION 1..372: note = Synthetic
VAEYITLRLPFHVTGVDAKLIYWTAWLCKLCAHRLLEAIKLNPSLAYLSQYDFIKVGRRLCYDIIPNRRYVDGISTLIHASLQSAKALKVDITKLEPKHWLLFQSEAEPWAKGNLNIQFVDYDKVKVLVFDRDKSSRRIIIEPVIPKGYIRLVKALVDKALKKEIGYPARIYITDYGERLEHLYGEIQVMVKYNFYLEVMKRYDKPLGDNIAGVDVNVDRLNLVVINRDGDIVWKHTVRFPQITSRGCPRKRAWSIIGEAIHSILRNAYSHGASVVAVENPEVLGYLRYYWIKNGDRGTKNYNYRISIFRNSIIERIVWKAPLYGLHVVTVNPKGTTHSKDHEYVMRRYGLDRHTASAYLIALKTRRNLQRP

SEQ ID NO: 78 - protein, 375 aa, synthetic construct
  REGION 1..375: note = Synthetic
VKMPRYVTIGLDFHVDGEDAKAILSTAWLSKLVAHRVLNLVKENELLAGLSEYMFLKVMRRKCYSILPNRRYVDGMLKLIHSIIKSAKALGVDIESIELKQWLLFQSDGERGYARGNMNIKLIDTHHARVLTLDYTGKTRYEELKLTTPKGWNKLVESLIERASRGEIGYPVRLVIRDYGIGKHGLHVHGEVQVMAPYTLYLEVMRRYEEPRGKNIAGIDVNVGRLDAVVVDEHGNLLSYRTFWIKNATFMGVRRRRSRSLIGEQIHALLKWLYSQGVSIIGLENPEVIGYLRYYWVRSGERRGNMWNWKISMFRNSIIERLTWKAPLYSMKTIYVNPRNTSKEGKRIGKKLGLDRHLGSAYIIARRTLKNLQRP

SEQ ID NO: 79 - protein, 375 aa, synthetic construct
  REGION 1..375: note = Synthetic
MELPKYITLTIDFHVSGDYARNILSTAWLSKLVAHRVLRLIKENEVLADLSEYKFLKILRSKCYDILPNRRYVDGILKLVHSTLKSAKALEVNMKDIGLKQWLLFQSDGERGYARGNMNIRFIGTHRVRVLTLGYDKKTRYIELKVPTPKGWKKLIEVLVNRAFDGQIGYPARVVVKDYGIGKHGLHVHGEVQVMVPYSLYLDVMRRYDKPRGDKIAGVDVNVERLDAVVIDRHGNLLSYGTFWLKNATFMGVRRKRSWSLVGEKIHALLKWLYSQGVSVVGLENPEVIGYLRYYWIRNGERRESRWNWKVSMFRNSIIERITWKAPLYSIKPTYVNPKNTSKEGERIGRRLGLDRHLGSAYIMAKRTLKSLQRP

SEQ ID NO: 80 - protein, 374 aa, synthetic construct
  REGION 1..374: note = Synthetic
MKQIEYITLRLPFHVTGGNAKLIYWTAWLCKICAHRLLNIIKENPLLADLSQYDFIKFSRRLCYDIIPNRRYIDGISTLLHASLQSAKALKVDITKLELKPWLLFQSEAEPWAKGNLNIQFIDYNKVRVLVFDKDKSSRKITMKPVIPKGYAKLTRSLVEKALRKEIGYPARIYITDYGGGLEHLYGEIQVMVKYDFYLEAMKRYDKPPGNNIAGIDVNVDRLNLVVINRSGDIVWRYTARFPQVTSRGYKRKRAWSIIGERIHEILRNAYSHGASVIAVENPEIIGYLRYYWIRNGERKTKNYNYKVSIFRNSIIERIMWKAALYGLQVITVNPKGTTHSNNHEYVMRKCGLDEHTASAYLIALRARRNLQRP

SEQ ID NO: 81 - protein, 369 aa, synthetic construct
  REGION 1..369: note = Synthetic
ITISLEFHTDDEEARAVLTTAWFSKLVAHRALRLVKENEVLADLSEYKFLKVMRSKCYDILPNRRYVDGMLKLIYSTLKSARALRVDIEKVELKQWLLFQSDGERGYARGNMNIRLINTHHVRVLTLDYDRKTRYVELKLAIPKGWRKLVEVLVEKATKGLIGYPARIIVKDYGIGKRGLHIHGELQIMIPYSLYLEVMRRYDKPRGNNVAGVDVNVDRLDAVVIDIYGNLLSYKTFWLKNSTFMGVRRKRSWSLIGEQIHALLKWLYSQGVSVIGLENPEIIGYLRYYWIRNGERKGRKWNWKVSMFRNSIIEKITWKAPLYSMKTIYVNPRNTSKEGEKIGKRLGLDRHLGSAYIIAKRALKYLQRP

SEQ ID NO: 82 - protein, 337 aa, synthetic construct
  REGION 1..337: note = Synthetic
MGKEEARATIKGSLELGDLERAKLLYTMRLFKEAVEKAHHLIKEGMPEGEIYKRLYYTIPNSHYCQAAYRKARLYEPQCHLSLKKPLLFSHGKGAGREDGNRNIKIESERIRIKIPHANGKHEWIEGKLRTQKRYKPILEELKNYSYSAGVAMNKGGYSVYISIPIELYAKISGKKHIRSDAKHIAAFDLNSDRLNMALLRANGLLIETKNRHFPEVNSQGYGEGKAKDKRLKALSELIDYATVHGAKTFVFEKLKMNGRKNISTKTGNRKLTRFAAHELLSHAEIMVKKRGGEFVQINPAYSSQSARIINQDLGVDVHTASAVVLGLRYLHTHKYT

SEQ ID NO: 83 - protein, 49 aa, synthetic construct
  REGION 1..49: note = Synthetic
MGWIRLRISDEVEREFRIKVAELGGKKGDLSRYFEEAVKEWVKKKRERQ

SEQ ID NO: 84 - protein, 339 aa, synthetic construct
  REGION 1..339: note = Synthetic
MDRKTEEARATIKGKLELEDLERAKVFYTMRLFKEAVEKAHHLIKKGLSKGEIVKSLYWTIPNTHYCEAAYAKAKLYSEQNHLKLKNPLLFSTGKGAGREEGNRNIKIKGNEVKIKIAHADGKHEWVTGKLIIQKRYLPILNELEDYGYSAGISLKDNRYRLHISVPVELYAKKDNVKRVKKEAKNIASFDLNSDRINMTLMRSNGLLIEARNKNFPEVNSPGYSENKAKDTRLKALSELIKYATAHDVKTFVFERLKMKGAKNISSKTGNRKMSKFAAREFLSHAEIMIRKRGGEFKQVNPAYSSKNARIINQDLGMDVHTASALVLGLRYLHTPKNI

SEQ ID NO: 85 - protein, 52 aa, synthetic construct
  REGION 1..52: note = Synthetic
MGIINVKVDDEVEMAFRRGVLEAKGKKKGALGKGIEEAMKIWIEKQKKQEQQ

SEQ ID NO: 86 - protein, 335 aa, synthetic construct
  REGION 1..335: note = Synthetic
VAREEGRATIKGKLELGALERAKLLYTMRLFREAVEKAHHMKKKGAAEEEIRKALYWTIPNAHYCDSAYKRALLYASQEHLKLRKPLLFSVGKGAKRENGNRNIQIDGERIRIKIPRADGKHEWIEGKIKTSKKYLPILKELRNYSYSAGISLRDGKYYVYLSVPMELYARVGEKEKVREDAEHIASFDINSDRINCVLLRKNGELIEVRNHRFPEVNGPGFPKEKARDTRLKALAELIDYAAAHGVKTFVFEQLSRKNKKDTGSKTGNRKLSRFALRELLSHAEVMVRKRGGEFKQVSAAYCSKIAKIIHHDLGMDVHTASALVLGLRYLRALN

SEQ ID NO: 87 - protein, 49 aa, synthetic construct
  REGION 1..49: note = Synthetic
MGWVRVRLSDEVERRFRIKVAELGGKKGDLSRYFEEAVKEWLEKREERR

SEQ ID NO: 88 - protein, 385 aa, synthetic construct
  REGION 1..385: note = Synthetic
MSDVVGASTVVDYVTIIGKLYPVSSEDYMRLVRLAWDFKNAVRITTRMIAKGVDKNSILRELRGMLNKAYADSAYKVARAIVEGCLFNGCNPLHIEVRRPFIVSEGESSRLGNRNVRLENTGLVKVKYPYDGSWITLRTEFGERYLPLLRELVDLARQREVSYGARIVFRGGSIYLHLSIPIKLYLKHFRKGEAEGGFVGGFDLNSDRVNLVIIDEHGRIVDTRTAWFTETTSHGFPRNRARALRLRALAKLLKYCYTHNVGVVVFENLPLIKRRRFTRSRVGNRKITRFAKRQLIQYGIVMALKYGFKTLLTNPRGTTNSKEHEEVMRKYGLDRHTASAYLTALRGLKALINMYTTNNHKNQNSPRVNWPSHIYPQQNQTLNRN

SEQ ID NO: 89 - protein, 48 aa, synthetic construct
  REGION 1..48: note = Synthetic
VPEEYVFHARISKTSHGLLCIYIPKRLSVRMMHLHGREVIIRVTVPDE

SEQ ID NO: 90 - protein, 352 aa, synthetic construct
  REGION 1..352: note = Synthetic
MSDDVVDATLLIKYVTVIGRLVPISSEDYMKLVRLAYDFKKAVLYATRMIAKGIDANSILRELRSMLNKAYGDSAYKVAKAIVEGCEYSNGNPRHIKIKRLFIVSDGEASRLGNRNIRLESVGVVKIKYPYDGSWLTFKALFGREYLPLVAELVELARQKRVSYGARIVFRSGRIYLHISIPLELYLKHFKRGEAGGSLVAGFDLNSDRINMVIVDKYGRIRDTKTAWFPEVTSHGFPRNKAKTRRLEALSKLLRYAYYHGVGVVVFENLFAVKKRVFGRNRIANRKIARFAKGELLRHGLVMAMKYGFRVYLANPKGTSNSREHDEVMRRYGLDRHTASAYLIALKGLKQQ

SEQ ID NO: 91 - protein, 48 aa, synthetic construct
  REGION 1..48: note = Synthetic
MPEEYVFHARISKTSHGLLCIYIPKGLSSKMQHLHRREVIIRVTVPDE

SEQ ID NO: 92 - protein, 329 aa, synthetic construct
  REGION 1..329: note = Synthetic
MRLVRLAWDFRRAVHVATRMIARGLEENSVLKELRRVLNKAYADSAYKTAKAMVDGCVFNGGNPLHIEVKKLFIISEGESSRLGNRNVRLEDTCLVKVKYLYDNSWITLQVEFGEKYLPLLRELVDLAKQKKVSYAARMVFREGNIYLHLSIPTQLYLKYFKKGGGKGRFIAGFDLNSDRVNLVIVDKYGRIVDARTAWFPGATSHGFPREKAKMLRLSALARLLEYCYRHGVSVVVFENLLSVKRKRFTRSKKANRKITRFAKKQLIQHGVVMALKYGFRVLLVDPKGTTNSRKHCELMKRYGLDRHTTSAYLIALKGSNTNEYECHK

SEQ ID NO: 93 - protein, 48 aa, synthetic construct
  REGION 1..48: note = Synthetic
VLEEYVFHARVSKTSHGLLCIYIPKELSEKMKHLHRRSVIVRVTVPDK

SEQ ID NO: 94 - protein, 356 aa, synthetic construct
  REGION 1..356: note = Synthetic
VSRCQTSSVVDASSIVNYVTITGRVYPVSSEDYMRLVRLAWDFRRAVHVATRMIVKGLEEDRILRELRRMLNKAYADSAYNAAKAMVEGCIFNGGNPLHIEVRKLFIISEGESSRLGNRNVRLEDTCLVKVKYPYDSSWITLRAEFGEKYLPLLRELVDLAKQKKLSYTARIVFREGIIYLHLSIPIQLYLKYFKKGEARGELIAAFDLNSDRVNLVIIDEHGRILDTRTAWFPEATSHGFPRGKAKALRLNALARLLEYCYRHSVSIVVFENLLSVKRKRFTRSKIANRKITRFAKKQLIQYSVVMALKYGFRVFLVDPKGTTNSREHYDVMRKYRLDRHAASAYLIALRGLRHQ

SEQ ID NO: 95 - protein, 48 aa, synthetic construct
  REGION 1..48: note = Synthetic
MPEEYVFHARISKTSHGLLCIYIPKELSEKMRHLHRRLVVVRVTVPDK

SEQ ID NO: 96 - protein, 362 aa, synthetic construct
  REGION 1..362: note = Synthetic
VSRCQTSSVIDASSIVNYVTIIGRVYPTSSGDYMRLVRLAWDFRRAVQVSTRMIARGLEENSVLRELRRVLNKAYADSAYKVAKAIVEGCIFNGGNSLNIEVKKPFIVSEGESSRLGNRNVRLEDTCLVKVKYPYDGSWITLRAEFGEKYLPLLRELVDLARQKKLSYTARIVFREGDIYLHLSIPTQLYLKYFKKGEARGRLIAGFDLNSDRVNLVIIDEHGKIVDVKTAWFPEVTSHGFPREKAKALRLNALARLLEYCYRHGVSAVVFENLLSVKRKRFTRSKKANRKITRSAKKQLIQHGVVMALKHGFRVLLVDPKGTTNSREHGEIMKRFGLDRHTASAYLIALRGLRHHEYGCYK

SEQ ID NO: 97 - protein, 48 aa, synthetic construct
  REGION 1..48: note = Synthetic
VPEEYVFHARVSKTSHGLLCIYIPKELSEKMRHLHRRLVVVRVTVPDK

SEQ ID NO: 98 - protein, 356 aa, synthetic construct
  REGION 1..356: note = Synthetic
MELPESVTIVGRIEFPDKKSYLELGKLCYLFKKNLQKAVKMYARGIRRDIIIKVVTKELNLGYADTIYKLARFIVEGCMFNNGEPFKIRIRKVFLASRGFSSNKGNRNIRLISTSEVLVRTPDRKWIKLYVKFGKKYLPLVKELVDLALKKKISYFARIVFRDGKAYLHISIPIQLYLKHFRLGLARGKLIAGFDINSDRINMVVIDENGCIRDVKNKHFPEVVSHGFPSNKARYLRLNAIAELLDYAHHHGVKYICFENLDVIKKKKFTKNKKANRKISKFAKREILTHAVIRALRYGFRILFVNPKGSSKEARKIHKKLGLDIHTASAYIIAIRGLKILKENESVENNEKIKLW

SEQ ID NO: 99 - protein, 72 aa, synthetic construct
  REGION 1..72: note = Synthetic
MSEVYAFRSTVTKDRDGAFKIRIPKHLTEKFSKLYRKNVIVVVLTDLGGKYGASRIRDNSGENRISRQEVLS

SEQ ID NO: 100 - protein, 359 aa, synthetic construct
  REGION 1..359: note = Synthetic
VSQYQTSSTVVDASTLVEYATITGRLIPASSEDYMKLTRLAWSFKRAVQVATRMIAKGVDSNDVLKELRKMLNKAYADSAFKIAKAIVRGCITNGGDPLHVEVRKLFIVSEGESSRLGNRNVRFEDTNLVKVKYPYDGSWTQFRAKFGEKYLPLLRELVKLAKQKKASYNAKIVFRNTKIYLHLSVPIQLYLKYFKRGDALGEFIAGFDLNSDRVNLVIIDKYERIVDVKTEWFPEVTSPGFSREKARALRLRALAKLLKYCYTHGVGAVVFENLLDVKKRRFAKSKTANRKISRFAKRELLHYVIVMSLKYGFKVLLADPKGTTSSAEHGRFMMKHGLDRHTASAYLVALRGLTHQIT

SEQ ID NO: 101 - protein, 52 aa, synthetic construct
  REGION 1..52: note = Synthetic
MVPVVPEEYVFHARISRTSHGLLCIYIPKELSEKMKHLHRREVIVRVTVPDK

SEQ ID NO: 102 - protein, 377 aa, synthetic construct
  REGION 1..377: note = Synthetic
MAGEVREPEVTHVTVTGRITPLNSEDHVKLCYLAYRFRKNLVRAVKMYARGVDKQVIVKEITRELNLGYADTIYKMAKLIVEGARGNGSKPLKIKVRKLFIASRGFSANKGNRNIRLESTNTLYVNIPREGWVKFKVSFGEKYIPLVKELIELAKSRKYSYGARIVFRNGRIYLHLSVPVELYLKYFGKGEARGSLIAGFDLNSDRVNMVIVDKSGVIRDARTEWFPEVTSHGFPRNRAKVARLQALSKLIEYAYHHGVGVAVFEDLDRIKRRRFTCSPVANRKMSWFPKRKLLGHGVVMALKHGFNVYLVDPSYTSVVGGYLSRELGLDKHTASAYMLTAKYLGLDLKSLKVTQITRKPKTKQHTDSPRLLYLAVI

SEQ ID NO: 103 - protein, 117 aa, synthetic construct
  REGION 1..117: note = Synthetic
LVFLDTGSFHLFISFLPAYGSRVVLGVSGAIGEERFTEPRPSPTAHQLTGSEIFKYPDALIYFSVVCLGKLYVRVDDDLETKFRVAVLRVKGKKRGALSEAVEEAIKLWLEKYESQR

SEQ ID NO: 104 - protein, 352 aa, synthetic construct
  REGION 1..352: note = Synthetic
MSKTEVVDASELVKYVTITGKLVPISIEDHMKLARLAHDFKRAVLVATRMVAKGVREAEVLRELRKRLNKAYADSAFKTAKAIVEGCSFNGGNPLHIEVRKLFIVSEGEASRHGNRNVKFESTDVIKVKYPHDGSWLSLRAEFGEKYLPLVRDLVDLAERRRISYHAKIVFRSGKVYLHLSIPIQLYLKYFKEGEARGRLIAGFDLNSDRVNLVIIDEHGEIVDVRTAWFPEATSHGFPREKAKALRLNALARLLKYCHRHGVSVVVFENLLSVKRKRFTRSKKANRKINRFAKKQLIQHGVVMALKYGLRALLVDPKGTTNSREHGELMKKYGLNRHTASAYLIALRGLRH

SEQ ID NO: 105 - protein, 48 aa, synthetic construct
  REGION 1..48: note = Synthetic
LPEEYVFHAKISKTSHGLLCIYIPKDLSQKMRHLHKREVIVRVTVPDE

SEQ ID NO: 106 - protein, 358 aa, synthetic construct
  REGION 1..358: note = Synthetic
LVNGADVVRYVTVTGRLVPLTPEDYMKLARLARDFRRAVVYATRMIAKRLDRSEVLRELRGMLNKAYGDSAYKVAKALVEGAKFNGGDPRCIEVKRLFIVSEGEASRLGNRNVRLESTDTVKIKYPYDGSWLTFRACFGEEYLPLVRELVELARARKASYGGRIIFRSGRVYLHLSIPVELYLKHFRRGEARGNLVAGFDLNSDRINMVIVDRYGRIRDIKTAWFPEVTSHGFPRNKAKARRLEALAKLLEYAYYHSVGTVVFENLLAIKHRRYTKSPSANRKITRFAKRELLQHAIISAMKYGFKVLLTDPRGTTRSRGHDEIMRRHGLDRHAASAYLIALRGTEGYRKYNLRFQPT

SEQ ID NO: 107 - protein, 55 aa, synthetic construct
  REGION 1..55: note = Synthetic
VGVVGAVMPEEYVFHARISRTSHGLLCIYIPKELSEKMRHLHRRRVIVRVTVPDK

SEQ ID NO: 108 - protein, 390 aa, synthetic construct
  REGION 1..390: note = Synthetic
MNTTYKCCYAISPNLKGVMCMDIRTPKIDYVTISGRVYVYNEDYQRLVYLAYRFRKAVVKATRMLTKGLSKEYVTKVITEDLNQGYAKSVVDIAKLLIKSAEYHGSNPLKIKVKKLFIVSKGEAFIKGNQNIRLVSSDKLLISYHLNGRHGRHDDWIECEARFGEEYIPLVNEVIDRAMNNELSYNAKILFRNGKIYLHLSVPLELYIKYFKKGEAKGGNIACFDLNSDRINMVIIDRQGIIKETKTEWYSEVNRPKYPKNKAWPLRLQALGRLLKYAYYHNVSTVLYEDLDRIKRRRVKKTKNRSGNRKASNFPKKKLLQYAITMALKYNFKVYLVNPSYTSRLAEKLKNAFCLDKHTVSAYLLGLRYLNPETYKKLLDRDTQKLLLNQ

SEQ ID NO: 109 - protein, 55 aa, synthetic construct
  REGION 1..55: note = Synthetic
LIGLLQAPGLQQATSPHELDGGIALQRHTMFSRGLFHGLSTYIYKLDKFVGSRPS

SEQ ID NO: 110 - protein, 356 aa, synthetic construct
  REGION 1..356: note = Synthetic
  SITE 42: note = misc_feature - Xaa can be any naturally occurring amino acid
VAGEIREPGVTHVTVTGRVVPLDSNSYMELVLLAHRFRKALXKAVKMRARGVDKNAIVKEITRELNLGYADTIYKLAKLVVEGARRNNSNLLKVEIRKLFVASRGFSSNKGNRNIRLLSTSELEVNIPWRGWVKFRVLFGGQYIPLVSELIEKALRRELSYTAKIVFRNGRPYLQVSTPIELYLKHFKKEVARGDLIAGFDLNSDRINMVIVDKLGVIRDVKTEWFPEVTSPGFPREKANTLRLQALAKLLGYAYHHGVGVVVFEDLERIRGRRYTKSRSANRKIGRFPKRKLLQYAIVMALKYGFKVYLANPAYTSKIGEKLGKELGFDKHTASAYVLALKALQPENYKLLTELR

SEQ ID NO: 111 - protein, 50 aa, synthetic construct
  REGION 1..50: note = Synthetic
  SITE 19: note = misc_feature - Xaa can be any naturally occurring amino acid
VGRLFVTIDDVVEERFRRXVARVYGVKKGALSRAVEEAIKLWLEKYESQE

SEQ ID NO: 112 - protein, 319 aa, synthetic construct
  REGION 1..319: note = Synthetic
MYAKGINRITIVKEVTKELNLRYADTIYKLAKLIVEGAKCNNSNPLKVKIRKLFIASRGFTVNKGNRNIRLLSSNELEINILWKGWIRFKVLFGRKYIPLVNELVKKALAKKLSYTAKIAFKNGNVYLHVSVPIELYLKYFSKGKAEGKLIAGFDLNSDRINMVIVDKYGIIRDVKTEWFPEVTSHGFPRNKAHTLRLQALAKLLDYAYHCSVEVVVFEDLERIKKRRFTMSRTANRKITRFPKRKLLKHAVVMAMKYGFKVHLVNPANTSKLGEKIGEKLGLDKHTASAYILTLKYLGVANLISCNPSSQRNQTTINY

SEQ ID NO: 113 - protein, 50 aa, synthetic construct
  REGION 1..50: note = Synthetic
VGILHVRIDDELEKKFRKKVLDVYGARKGALAKAVEEAIKLWLEKYESQS

SEQ ID NO: 114 - protein, 356 aa, synthetic construct
  REGION 1..356: note = Synthetic
MAGEVREPEITYVTITGRVIPLDNESYMELVLLAYRFRKALIKAIKMYAKGLDRNTIVKEITRELNLGYADTIYKLAKLIVKGAKHNNSNPLKIKVRKLFVASRGFTSNKGNRNIRLLSTSELQVNIPWKGWVKYRALFGKRYIPIVRELAEKALSKRMSYTARIVFRNGKIYLHLTVPIELYLKYLKKGEAKGGLIAGFDLNSDRINMVIVDKHGVIRDVKTEWFPEVISHGFPRNKANTMRLQALTRLMEYAYHHGVDTVVFEDLKRIKKRRCTRSKTANRKITRFPKRKLLEHGVVMARKYGFKVYLVNPAYTSKMGEKLGKILGLDRHTASAYILVLKYLGISKIPLSISSS

SEQ ID NO: 115 - protein, 52 aa, synthetic construct
  REGION 1..52: note = Synthetic
MLVGILHVRIDDELEKKFRKKVLDIYGVKKGALAKAVEEAIKLWLEKYENQK

SEQ ID NO: 116 - protein, 355 aa, synthetic construct
  REGION 1..355: note = Synthetic
VSRCQTSSIIDASSIVNYVTIIGRIYSASSEDYMRLVRLARDFRRAVQVATRMIARGLEENSVLRELRRMLNKAYADSAYKAAKTMMEGCIFNGGNPLHIEVKKPFIVSEGESSRLGNRNVRLEDTCLVKVKYPYDGSWITLRAEFGEKYLPLLRELVDLAKQKKLSYTARIVFKEGDIYLHISIPIQLYLKYFKEGEARGQLIAGFDLNSDRVNLVIIDEHGEIVDARTAWFPETTSHGFPREKAKALRLNALARLLKYCYRHGVSVVVFEDLLSVKRKRFTRSRTANRKITRFAKKQLIQHGVVTALKYGFKVLLVDPKGSTNSREHDELMRKYRLDRHTASAYLIALRGLKH

SEQ ID NO: 117 - protein, 48 aa, synthetic construct
  REGION 1..48: note = Synthetic
VPEEYVFHARISKTSHELLCIYIPKELSEKMRHLHRRLVVVRVTVPDK

SEQ ID NO: 118 - protein, 355 aa, synthetic construct
  REGION 1..355: note = Synthetic
MAGEIREPEVTHVTVAGRVVPLDSYSYMELVLLAHRFRKALTKAAKMYAKSMDKNTIVKEITRELNLGYADTVYKLAKLVVEGARRSNSNPLKVEIRKLFVASRGFSSNKGNRNIRLLSTSELEVNIPWRGWVKFRVLFGGQYIPLVSELVEKALRRELSYTAKIVFRNGRPYLQVSVPIELYLKHFKRGVAGGKLVAGFDLNSDRINMVVVDKLGIIRDVKTEWFPEVTSPGFPREKANALRLRALARLLGYAYYHGVGVVVFEDLERIKGREYTKSTSANRKIGRFPKRKLLQHAIVMALKYGFKVNLVNPAYTSKIGEKLGKELGFDKHTTSAYLVALKALQSENYKLLTEL

SEQ ID NO: 119 - protein, 50 aa, synthetic construct
  REGION 1..50: note = Synthetic
VGRLFVVIDDVVEERFRRVVARVCGAKKGVLSKAVEEAIKLWLEKYESQK

SEQ ID NO: 120 - protein, 356 aa, synthetic construct
  REGION 1..356: note = Synthetic
MAREVREPEITHVTITGRILPLDNDSYMELILLAYRFRKALIRAIKMYAKGLDRNTIVKEITKELNLGYADTIYKLAKLIAEGAKHNGSNPLRVRIGKLFIVSRGFSSNKGNRNIRLLSTNELQVNIPWKGLLRFKVAFGERYISLVSELVKKALSRKLSYTAKIVFRNGMVYLHVSVPIKLYLKHFSKGRAKGSLVAGFDLNSDRINMVIVDKLGIIRDVKTEWFPEVTSHGFPRNKAHTLRLQSLAKLLNYAYHHGVGVVLFEDLDRIKKRRYTRSRTANRKITRFPKRKLLEHSIVTALKNGFKVYLVNPAYTSKLGEKLGTELGLDKHIASAYVLVLKYLGVSEIPLSISSS

SEQ ID NO: 121 - protein, 156 aa, synthetic construct
  REGION 1..156: note = Synthetic
MPRSKEGSRFHMFMVNYIAEHLRKSGYKVSKFVRLPNNGIIDVYAERNGEIIIAECILRPTRRLIEEKIRKYKKFGKLIIVIPESYFNIDIKEENVEIWRIPVTINKKAILVEIDDELEMKFRIVVLKVKGKKRGALSEAVEEAIRLWLEKYGSQK

SEQ ID NO: 122 - protein, 357 aa, synthetic construct
  REGION 1..357: note = Synthetic
MDVPKYVTVCGRVIFDNNETYLELGKLCYLFRKNLQKAVKMYAKGLSKNIIVKTITKEMNLGYADTIYKLAKFIVKGCRANGGNPLRIRIKRMFLVSRGFSSNKGNRNIRLVSTREVLVRTLDKRWLKLGVKFGKKYLTLIEELVKLALQKRASYSARIIFRNGKAYLSVSVPIQLYLKYFKLGKAKGKLIAGLDINSDRINMVIIDDDGNIRDVKNEHFHEVTSQGFPRNKAKYIRLNAIARLLDYAYHHGVRYVCFENLDVIKKKKFTKSKKANRKITKFAKRQILSHAVIRAMRYGFKILFVNPKNSTKEAKKIHRKLGFDVHTASAYIIALRGLKILKNNGNFQNILTTQTSE

SEQ ID NO: 123 - protein, 54 aa, synthetic construct
  REGION 1..54: note = Synthetic
MSQIFVFRSTITKDKDGSYKVRIPKSISSKFDSLYKKGVVVIILTELGERYGRS

SEQ ID NO: 124 - protein, 363 aa, synthetic construct
  REGION 1..363: note = Synthetic
VLQYRISDVIATRLVTYTTIIGKLIPTSPENHMKLVRLAYDFRRAVLYATRMIGKDMKTNDILKELRNMLNKAYGDSAYKVAKAIVEGCRFNGGEPRHIKIKRLFIVSEGEASRFGNRNVRLETTDIVKVRYPYDKSWIILRTCFGRKYLSLVREVSELAKQRRISYGARIVFKDGRIYLHLSIPINLYLKYFSKGKAHGELVVGFDINSDRMNMVIIDRYGKIRDIKTKWFSEVTSHGYPKSKAKARRLEALAKLLKYTYHHNVGIVVFENLFTIKKREYSRNPTANRKITRFAKKELLRYGILMAMKYGFKVLLVNPRGTTRSKEHDEVMKKLGLDRHVASAYLIALRGIRRYTMIQKAMI

SEQ ID NO: 125 - protein, 48 aa, synthetic construct
  REGION 1..48: note = Synthetic
VSEEYVFHARISKTSHGLLCIYIPKELSEKMKHLHRRKVIVRVTVPDK

SEQ ID NO: 126 - protein, 358 aa, synthetic construct
  REGION 1..358: note = Synthetic
VLRYQTSSAVDASSIVNYVAIVGRVYPASNEDYMRLVALAWDFRRAVQVATRMIARGLEENSVLRELRRVLNKAYADSAFKTAKAMVEGCLFNGGNPLRIEVRRLFIVSEGESSRLGNRNVRLEDTHLVKVRYPHDGSWITLRAEFGERYLPLLRELVDLARQKKLSYKAIVVFREGGIYLHLSVPIQLYLKYFKKGEARGQFIAGFDLNSDRVNLVIIDEHGRIVDTRTAWFPEATSHGFPREKAKVLRLNALARLLEYCYRHGVGTVVFENLLLVKRRRFTGDRTGNRKIARFAKRQLIQHGVVMALKYGFRVLLVDPRGTSGSKEHDEIMKRFGLDKHVASAYLIALRGLRYQQI

SEQ ID NO: 127 - protein, 43 aa, synthetic construct
  REGION 1..43: note = Synthetic
VFHARVSKTSHGLLCIYIPKELSERMRHLHGRLVVVRVTVPDK

SEQ ID NO: 128 - protein, 181 aa, synthetic construct
  REGION 1..181: note = Synthetic
MAPYSLYLDVMKRYENPIGEHIGGVDVNVDRLNLAIVDKYGRLRDVKTFWFREITSRGYRRKQAWTKVHQAIHKLLDYAYQHGVSVIALESPKIVGHLKYYWIRNNTDRGSRNYNYRKAIFRNKIIEVTTYKAPLYSIKVIHVNPKGTTSSREHGEVAKKYGLDKHVASAYMVALKGLKQL

SEQ ID NO: 129 - protein, 183 aa, synthetic construct
  REGION 1..183: note = Synthetic
MVKYGFYLKVMRRCDKPLGRNIAGVDVNTDRLNLVVIDENGDIVWKYTAKFLQVTSRGYLRRRAWSIIGEAIHKVLKNAHSHGASVIAVENPKVIGYLRYYWIRNGDRKLENYNYKISIFRNSIIKRIMWKAPLYGLQVIAVNPKGTTHSEDHEYIMRRYGLDRHTASAYLIALKARRNLQRP

SEQ ID NO: 130 - protein, 201 aa, synthetic construct
  REGION 1..201: note = Synthetic
VVIKSYGVEEGDLWVQEEVQITIPIDFYYKHMSKARVNNGRLYGGVDVNVDRINLVIIDDSGSLRDIHTFWFEEASRKGCNKHRARSIIGMKIHKLLKHAYHHGVKTMFLENPEVLGRLKLFWIKSDDRKHKNYNYKRTIFRSSIIEMITLKAPLYGIKVKFVNPKGTTNSEEHDEIMKRYGLDRHVASAYLIALKGLRHQ

SEQ ID NO: 131 - protein, 276 aa, synthetic construct
  REGION 1..276: note = Synthetic
MVCWIERRIRFCTLRLRGSGFGSRGNKSDKGNRNVKFHVFQDHVEVRVKDPWGKWVHGEAYFGKEYLPLLHELEELAERKEEGYGAVVSFKHYPMIHLQIPLWLYLKHFSLPKPVGYGLVAGFDLNSDRLNVVVVNKDGKVVTKKTWWYSDVTRPGFPKGKAMALRLNALSQALNFLSRVGVDYVAFEDLFLVKKRRFTRSKSGNRKVSRFAKKQLLTHGVVKALRLGFNVVLVNPKGTTNSEGHDRIMREKGFDRHTASAYLIALKGLGMLNDIK

SEQ ID NO: 132 - protein, 253 aa, synthetic construct
  REGION 1..253: note = Synthetic
LPVGGIKLIRVNRNIKFRVLEDHVLIKVKDPWGTWVYGKAYFGKDYLPLLRELEDLSQRKEEGYGAVISFPRIHLQVPLWLYLKHFSLPKPNGYGLIAGFDLNSDRLNLVVIDKGGGAITIKTFWYSDVTRPGFPKEKARALRLNALSQALNFLSRIGVDYVVYEDLFLVKKRKFTRSRSGNRKVSRFAKMQMLIHGVIKALRVGFNVVLVNPKGTTNSEEHDRVMRKKGFDKHTASAYLIALKGLGMLNDIK

SEQ ID NO: 133 - protein, 237 aa, synthetic construct
  REGION 1..237: note = Synthetic
LEDHVLIKVKDPWGEWVFGKAYFGKEYLPLLKELEELSRRKEEGYGAVVSFKDYPMIHLQIPLWLYLKHFSSPKPVGYGLFAGFDLNSDRLNVVVVNKESRIVTKKTWWYSDVTRPGFPKGKAMALRLNALSESLNFLSRVGVDYVVFEDLFLVKKRKFTRSKSGNRKVSRFAKRQMLIHGVIKALRLGFNVVLVNPKGTTNSEGHDRIMREKGFDRHTASAYLIALKGLGMLNDIK

SEQ ID NO: 134 - protein, 233 aa, synthetic construct
  REGION 1..233: note = Synthetic
VLIKVKDPWGKWVYGKAYFGKEYLPLLRELEELSKRKEEGYGAVISFNERPMIHLQIPLWLYLKYFSTPKLAGYGLIAGFDLNSDRLNVVVVNKDGKVVAKRTWWYSEVVSHGFPKEEAMALRLNALSEALEFLSRIGVDCVVFEDLFLIKGRKFTSSKSGNRRVSRFAKRQMLTHGVIKALRLGFNVVLVNPKGTTNSEEHDRVMREKGFDRHVASAYLIALKGLEVIKNNE

SEQ ID NO: 135 - protein, 182 aa, synthetic construct
  REGION 1..182: note = Synthetic
MIHLQIPLWLYLKHFSSPKLVGYGLVAGFDLNSDRLNVVVVNKDGKVIAIKTFWYSDVTRPGFPKGKAMALRLNALSQALNFLSRVGVDYVVFEDLFLVKKRRFTRSRSGNRKVSRFAKRQMLTHGVIKALRLGFNVILVNPKGTTNSEGHDRIMREKGFDRHTASAYLIALKGLGMLNDIK

SEQ ID NO: 136 - protein, 182 aa, synthetic construct
  REGION 1..182: note = Synthetic
MIHLQIPLWLYLKHFSSPKPVGYGLFAGFDLNSDRLNVVVVSEDGEIIANKTWRYSDVTRPGFPKGKARALRLNALSQALNFLSRIGVDYVIFEDLFLVKKRKFTRSKSGNRKVSRFAKKQLLTHGVIKALRLCFNVVLVNPKGTTNSEDHDKVMREKGFDRHTASAYLIALKGLEVIKNNE

SEQ ID NO: 137 - protein, 182 aa, synthetic construct
  REGION 1..182: note = Synthetic
MIHLQVPLWLYLKHFSSLKPAGYGLIAGFDLNSDRLNVVAINKDGKVITTRTFWYSDVTRPGFPKEKARALRLNALSNALEFLSRIGVDYVVFEDLFLVKKRKFTRSKGGNRKISRFAKKQLLIHGVIKSLRLGFNVVLVNPKGTTNSEEHDRVMREKGFDKHTASAYLIALKGLGMLNDIK

SEQ ID NO: 138 - protein, 166 aa, synthetic construct
  REGION 1..166: note = Synthetic
SSPKPNGYGLIAGFDLNTDRLNVVINKESSIVTTKTWWCSEVVSHGFPKGEARALRLNALSQALIFLSRIGVDYVVFEDLFLVKRRRFTRSKSGNRKVSRFAKKQLLIHGVIKALRLGFNVILVNPKGTTNSEEHEKVMREKGFDRHTASAYLIALKGLGILNDGK

SEQ ID NO: 139 - protein, 141 aa, synthetic construct
  REGION 1..141: note = Synthetic
MVIVDKCGRIRDTRTAWFPEVTSHGFPRNKAKTRRLEALSELLRYAYYHGVGVVVFENLFAVKKRVFGRNRIANRKIARFAKRELLRHGVVMAMKYGFRVYLANPKGTTSSKEHDEVMRRYGLDRHTASAYLIALKGLKQQ

SEQ ID NO: 140 - protein, 162 aa, synthetic construct
  REGION 1..162: note = Synthetic
VGYGLVAGIDLNSDRLNVVVVNEEGKVVTAKTWWYSEVVSHGFPKDKAKALRLNALSQALNSLSRIGVDYVVFEDLFLVKKRRFTRSKSGNRKITRFAKRQMLIHGVIKSLRLGFNVVLVNPKGTTNSEDHDKVMREKGFDRHTASAYLIALKGLDAIKNNE

SEQ ID NO: 141 - protein, 157 aa, synthetic construct
  REGION 1..157: note = Synthetic
VLIAGFDINSDRINVVVIDVSGDIIALKSFWYSETVSPGFPRNKARLLRLNAVSNALKWCRGIRVDSVVFENLTKIKIRGFTENPYANRKIAKFAKKELIKHGVIKALKLRFAVILVDPKGTTNSLTHKQIMKEKELDNHAASAYIIAYRGLKRLKE

SEQ ID NO: 142 - protein, 242 aa, synthetic construct
  REGION 1..242: note = Synthetic
LEDHVEVKVKDPWGRWIVGRAYFGRKYLPLLRELEELAGKGEEGYGAVISFKEKPMIHLQIPLWLYLKHFSVKKPIGYGLIAGFDLNSDRLNVVVIDRDGRIVTTRTYWYPEVTRPGFPREKGKALRLNALSNALKFLSRIGVDYVVFEDLFLVKGRRRFTKSKSGNRRISRFAKRQLLTHGVIKSLRLGFNVILANPKGTTNSKEHEKVMKERGFDRHTASAYLIALRGLETNSIKGVRSN

SEQ ID NO: 143 - protein, 235 aa, synthetic construct
  REGION 1..235: note = Synthetic
MVKYPYDRSWLFFKAQFGEKYVSLLQELVKLSKERKLSYGARIVFRDGKIYLHVSIPIELYLKHFNKGSTNGVLTAGFDFNANRINMVIVDRYGRVRDIKTVRFPEVTSHGYPRNKARAKRLEALAKLLKYAYHHNVGTVVFENLFIVKHRKFTRNSTANRKISRFAKKELLQYGVVMAMKYGFKVLLVNPKGTTSSKEHDIVMKKYGLDRHTASAYLIALRGIERHTTIQKATT

SEQ ID NO: 144 - protein, 207 aa, synthetic construct
  REGION 1..207: note = Synthetic
LIKELVELAKAKRISYVARIIFHNGSVYLHASIPIGLYLKYFSKGRANGELIAGFDLNSDRINMVIVDRYGRIRDMKTTWFPEVTSHGFPKNKARARRLEALTKLLRYAYYHGVGTVVFENFLMIKHRRYVRNPTANRKITRFAKKELLQYAIVSAMKYGFEILLINPKGTTCSREHDEVMKRYGLDRHAASAYLIALKGIKRYEMT

SEQ ID NO: 145 - protein, 188 aa, synthetic construct
  REGION 1..188: note = Synthetic
MWLYLKYFSTPRQKGYGLIAGFDINSDRLNVVVINEVGVIIALKTFWYSEVVSHGFSKEKARWMRLNALSSALKWCRRIGVDYVVLEDLTRIKNRKFTSNPTANRKIAKFPKKQMLKHGVIKALKLGFTVILVNPKGTSSSVTHRQIMREKGLDRHMASAYMIAYRGLKKLEEQEPSISPSTTPTINQ

SEQ ID NO: 146 - protein, 237 aa, synthetic construct
  REGION 1..237: note = Synthetic
LDDHVEVKAKDPWGKWIYGKAYFGKEYLPLLHELEELASNKVEGYGAVISFKHYPMIHLQIPLWLYLKYFSSPKPNGYGLVAGFDLNSDRLNVVVVNKEGKVITTKTWWYSEVVSHGFPKGKAMALRLNALSQALSFLSRVGVDYVALEDLFLVKRRKFTRSKSGNRKVSRFAKKQLLVHGVIKALRLGFNVILVNPKGTTNSEEHEKVMMEKGFDRHTASAYLIALRGLEVIKNNE

SEQ ID NO: 147 - protein, 348 aa, synthetic construct
  REGION 1..348: note = Synthetic
MVRESEERATIVGKLLVNDDVALAKLLTLMRMFRDSVELAHTLLFRRKFSESEVKRRLTKVLSNAWYASSAIKVAKLYKEQTKIKLRKPLLYSVGAKCEEGNRNIRLVSTDKVLIKIPHADGRHEWIEVRVRFGKNYIPLLQELNSCNYGAGVTIKLKGKRENWKTVFRKKLYLYLNVPADLYAKYFGKRVKVESENKFYAGFDFNVDRINMVIVDSYGRIRDIRNIHFPEVINYDKNKSRAIRQDALSKLVEYAVSHGVKYFVIEDLSKPNNIRGNVRKWSIRKYQQQVSMLVKKVGGTLVKVNPAYTSIDAIGISLSKGIDVHTASAYLIALRGIKRYTTIQKATV

SEQ ID NO: 148 - protein, 50 aa, synthetic construct
  REGION 1..50: note = Synthetic
MVRKTVEIPDELWREFEVHAVRKFGYYGAIKKALEEAIRLWLEKVKKEQQ

SEQ ID NO: 149 - protein, 350 aa, synthetic construct
  REGION 1..350: note = Synthetic
MAREGEEKATVVGKLILNDEVSLARLLTIMRQFRDAVELGHSLLFREKLSESEVKRRLTRILSNAWYAYSAIKVARLYKEQSKIRLRKPLLYSVGARCEKSNRNIRLVSTDKVLIKIPHASGKHEWIEVGVRFGKKYLPLMRELISGSYSYGAGVTIKLKGKKEDWRRVFREKLYLYLNIPVSLYVKYFSKRVKVKPRNRFYAGFDFNVDRINMVIVDSYGRIRDVRNIRFPEVVNYDKDKSRAIRQEALSKLVEYAVSHGVKYFVIEDLSKPNKIRGKIRKWSVREYQQQMEMLIKKVSGTLIKVNPVYTSIDAIGIALSRRIDIHTASAYLIALRGIKGYELIQKTII

SEQ ID NO: 150 - protein, 53 aa, synthetic construct
  REGION 1..53: note = Synthetic
VYEMVRKTVEVPDELWREFEVYAVRKFGYYGAIKKALEEAIRLWLEKVKKKLQ

SEQ ID NO: 151 - protein, 348 aa, synthetic construct
  REGION 1..348: note = Synthetic
MARGSEEGYVTIVGKLIVSDGVSASKLLLLMRRFRDSVEMAHNTTSKRQLGESEVKRRITRYLSNAWYASSAIKMSKLYEEQDHLRLRKPFLYSLGSRDENGNRNIKFVSTDRVLIKIPHEDGRHEWIECKALFAKKHIPVIEELLRGRYSYSAGVAIKLRKGEDWRSVWRKRLVIYISIPVELYIKHVKPKNVDVAGTVNSNWLLAGFDFNVDRVCMAIIDPAGRLRDARTEFFSNAANTLKEVSENARKVAVVKLVKYAVAHGVRYIAVEGLRRPSRVEGRVGKWAHRQYLQHLKVLAKRFGLEVIEVNPAYSTIDAIGIALTLGLDKHTASAYIIALRGLAHKSM

SEQ ID NO: 152 - protein, 76 aa, synthetic construct
  REGION 1..76: note = Synthetic
VTCYIFWFLRFILYIVLYLVYDLSERVYVKKTVTVPLDLWREFEVEATRKFGYYGAIRKAVEEAIKLWLEAQRKAT

SEQ ID NO: 153 - protein, 274 aa, synthetic construct
  REGION 1..274: note = Synthetic
MSKLYEEQDHLRLRKPFLYSLGSRDENGNRNIKFVSTDRVLIKIPHEDGRHEWIECKALFAKKHIPVIEELLRGRYSYSAGVAIKLRKGEDWRSVWRKRLIIYISIPVELYIKHVKPKNVDVAGTVNSNWLLAGFDFNVDRVCMAIIDPAGRLRDARTEFFSNAANTLKEVSENARKVAVVKLVKYAVAHGVRYIAVEGLRRPSRVEGRVGKWAHRQYLQHLKVLAKRFGLEVIEVNPAYSTIDAIGIALTLGLDKHTASAYIIALRGLAHKSM

SEQ ID NO: 154 - protein, 66 aa, synthetic construct
  REGION 1..66: note = Synthetic
MARGSEEGYVTIVGKLIVSDGVSASKLLLLMRRFRDSVEMAHNTTSKRQLGESEVKRRITRYLSNA

SEQ ID NO: 155 - protein, 348 aa, synthetic construct
  REGION 1..348: note = Synthetic
  SITE 310: note = misc_feature - Xaa can be any naturally occurring amino acid
LARGAEEERVTVVGKLVIQDGVSASKLLLLMRKFRDAVEMAHSMLYKKQLSETEVKRRLTRYLSNAWYANSAIKVAKLYREQDHIKLRKPLLYSVGSSDEKGNRNIKFTSTDSVLVKFPRESGSHEWIECKVLFGKKQLPVISELITGSYTYGAGISIKVKRSEDWRSMWKKRLIIYVNIPIELYAKHRRVGGVPTNSGAELWAGFDFNVDRVCVAIVDSAGRLRDVKTRLFSNAVNTPSEVSKTIREEALAELVKYAVAHGVKHLVVESLERPGRVKGKVGKWALRQYIQHLRVLARRHNVDVVEVSPXYSSIDAIGVALALGLDRHTASAYLIALRGLAHQIKKMK

SEQ ID NO: 156 - protein, 56 aa, synthetic construct
  REGION 1..56: note = Synthetic
MSEKKFVKKTVTVPADLWREFEVEATRKFGHYGAIKRAMEEAIRLWLEVQKKRESQ

SEQ ID NO: 157 - protein, 372 aa, synthetic construct
  REGION 1..372: note = Synthetic
VIKEAIINHVTVRAFSRISNRDVIAKLLFTARMYRLGVIIALECLEKNIPMKECMFKIKRFVSNQKYAHACYERAREMYKSAKKLGKEVRKLKQWLIFEAIGDKLDKGNRNIRLSSPNEVEVVVFTKNYGIERIRVPIKLLKTHVSIIRELFELGSNKLLGYNARCVIRDYSRDKALVEFQFTVPVELYLKYRRVYDKPRGRNIGGIDWNSDRANLAIVSSKGELLDHKTWWFPEVTSHGYPRTARRTQQSQILSEIMDYCYHHGCSVVVFEDPDIIKTRRCTKNATANRKITRWTKRDASQMFVLKALRYGLKPFFVNPAYTSKLAELIAKDVGLDRHTASAYILALEYLGLNPKEIYQNLHRPIKTFTLR

SEQ ID NO: 158 - protein, 51 aa, synthetic construct
  REGION 1..51: note = Synthetic
VSEVLLVFEGKISRWGRRGYVIYVPKEYERVVEKYYGEKVVVIILRKGENP

SEQ ID NO: 159 - protein, 365 aa, synthetic construct
  REGION 1..365: note = Synthetic
MIREAIIDRVTVRGFGKVRDSDAIAKLVFTARMYRLGVLTALECLESKIPMKECMFKIKRFVPNQKYAHACYERAREMYKSAKRKGRKVKQLRQWFMFESIGDRLDKGNRNIRLSSPNEVRVVVFAEKFGIKRIKLPIKPLKTHTEIIKELYELGSSKLLGYNARCIIRDYSRDEALVELQFIVPVDLYLKYRRIYDKPRGRNIGGIDWNSDRANLAIVSPKGELLDHKTWWFPETTSHGYPRIARKTKQSQVLSEIMDYCYHHGCGVVVFEDPNIIKTRRFTKNSTVNRKITRWTKREASQMFILKALRYGLKPFFVNPAYTSKLAGLIAKDLGLDRHTASAYILALEYLGLRPKEAYQNLQKP

SEQ ID NO: 160 - protein, 51 aa, synthetic construct
  REGION 1..51: note = Synthetic
VSEVLLVFEGKISRWGRRGYVIYVPREYERVAEKYRGEEVIVIVLRKGANP

SEQ ID NO: 161 - protein, 365 aa, synthetic construct
  REGION 1..365: note = Synthetic
VIKEAIINHVTVRAFSRISNRDDVAKLLFTARMYRLGVTIALEYLEKNIPMKDCMFKIKRFVPNQKYAHACYERAREMYKSAKRKGKKVKQLRQWLMFESIGDRLDRGNRNIRLSSPNEVEVVVFTKNYSIERVRVPIKPLKTHTEIIEELYELGSSKLLGYNARCIIRDYSRDKALVEFQFIVPLELYLKYRRLYDKPRGLNIGGIDWNSDRANLAVVSPKGELLDHKTWWFPETTSHGYPRTARRTKQSQILSEIMDYCYHHGCSVVVFEDPDTIKTRRYTKNATANRKITRWTKREASQMFILKALRYGLKPFFANPAYTSKLAEVIAKELGLDRHTTSAYILALEYLGLKPKEIYQNLQRP

SEQ ID NO: 162 - protein, 52 aa, synthetic construct
  REGION 1..52: note = Synthetic
LVSEKLLVFEGKVSRWGQRGYVIYVPKEYERVVKKYHGEKVIVIILRKGENP

SEQ ID NO: 163 - protein, 348 aa, synthetic construct
  REGION 1..348: note = Synthetic
MARRRRENSSEEKVTIKGRLVIEDRFALAKLLYTGRLFRDAVEMAYRLMKKGLKHEEATRRVTKFIGNAHYAYSATKRAMAYLDQERLNFKKPQLYSVGKGCEKGNRNIRLVSVDKVLIKIPHVDGRHEWLEAKVKFHSRHIPLIEELIQRANNGMPYSVSVVFNKGKYYLHLHIPLELYAKHTTKVKVSSKAHLIAGFDINPDRICMVIVDTNGNIRDVKTKHFPEVTLVGFPKNKAKDLRLKALSELINYACYHNVKYFVFEKIRKIPKKKMKTKSASRKVSRFAYAELLQHAKVMVRKKKGKFVQVNPAFTSVDAIPLSRKLGLDIHVSSAYLLAIRCLKSINAY

SEQ ID NO: 164 - protein, 48 aa, synthetic construct
  REGION 1..48: note = Synthetic
MKIAESRVSKRYLVAIPRAVRVALNIDVGDVIEWHVEDGKIVVKKKLQ

SEQ ID NO: 165 - protein, 342 aa, synthetic construct
  REGION 1..342: note = Synthetic
MARKARKEYLAVNGKLLIMDNIARSKLLYTMRLFRDAVKMAHYLTRKGLAWKDIEKRLTSFISNAHYGHSAYQRAKLWENQPYLKLRKKFLFSVGKSAEKGNRNIRLIPSDNYFIVKIKIPHANGRHEWIECKAKFGKRYIPLLKELSRRNVSYGVEIGEYYKIHVYVPLELYAKYLGKKVGGKGKSLHVAGFDLNTDRINMAIINSNGELLDFKVKHFPEVTQHGFPKEKAKDIRLKALSELIDYACYHDVEYFVFEKLGKIKRKKTSNKNINRKLSKFSKKQLLTHAKVMVAKRGKKFCEVNPAFSSQDAELIARKLRLDTHTVSAYILAYRALKSINVY

SEQ ID NO: 166 - protein, 50 aa, synthetic construct
  REGION 1..50: note = Synthetic
MSIIHVKIDKDLAIEFRRKVLEVKGVKKGALAEAVEEAIRLWLEKHGKST

SEQ ID NO: 167 - protein, 341 aa, synthetic construct
  REGION 1..341: note = Synthetic
MVRKVRKEYVAVNGRLLIADNIAKSKLLYTMRLFRDTIKMAHYLMRKELAWEDIEKRLTSLISNAHYGHSAYQRAKLWKDRPYLKLRKKFLFSVGKSAEKGNRNIRLIPSDNYFIVKIKIPHADGRHEWIECKAIFGKRYIPLLKELSKGNVSYGVEIGEDYKIHVHVPLELYARYLGKNISRRGKSLHIAGFDLNADRINMVIINSSGELLDFKVKHFPEVTQHGFPKERAKDIRLKALSELIDYACYHNVEYFVFEKLGRRRRKKTSNRNINRKLSKFPKKQLLTHAKVMVAKRGKKFCEENPAFSSQDAELIAKKLRLDTHTTSAYILAYRYLKSINT

SEQ ID NO: 168 - protein, 62 aa, synthetic construct
  REGION 1..62: note = Synthetic
VVSYISILVFRCMSIIHVKIDKELAMEFRRKVLEVKGVKKGALTEAVEEAIRLWLEKYGRST

SEQ ID NO: 169 - protein, 342 aa, synthetic construct
  REGION 1..342: note = Synthetic
MARKTRKEYVAVNGKLLIANNIAKSKLLYTMRLFRDAVKMAHYLMRKGLVWRDIEKRLTSFISNAHYGHSAYQRAKLWKDQPYLKLRKKFLFSVGKSAEKGNRNIRLIPSDNCFIVKIKVPHADGRHEWIECKAKFGKRYISLLKKLSEENVSYGVEIGEDYKIHVYVPLELYTKYLGKKISRKGKSLHIAGFDLNVDRINMVIINSSGELLDFKTKHFPEVTQHGFPREKAKDIRLKALSELIDYACYHNVEYFVFEKLGKVKRKRTSNKNVNRKLSKFPKRQLLTHAKIMVVKRGKKFYEVNPAFSSQDAELIAKKLRLDMHTASAYILAYRALKSINVY

SEQ ID NO: 170 - protein, 50 aa, synthetic construct
  REGION 1..50: note = Synthetic
MSIIHVKIDKDLAIEFRRKVLEVKGVKKGALAEAVEEAIRLWLEKHEKST

SEQ ID NO: 171 - protein, 343 aa, synthetic construct
  REGION 1..343: note = Synthetic
MVHRGGQSSSEEKECITIKGRLEFPDIVAKAKIFYTMRLFREAVERTFILFKKGLKDREAVKRVTRYMNNAHYSYSALQRAKMYANQNRLRLRKPQLYSIGKACEKGNRNIRLVSKDTVLIKMPHADGRHEWVECKVKFGKKHLPIIEELINAEFPYSVAVTLNNSKFYLHVAVPLEIVAKYDHHPKVSERSGYIAGFDLNSDRINMVIINEHGEMVDIKNKHFPEITQHGYSTNKTRDAVLKALSELIDYAAHHNVRYFTFEKLGNIKRKKTRNKNANRKISRFPYRTLLTHAKIMVKKRNGVFATVSPAYTSIDAIPLSRKLGLDIHTTSAYLLALRYLNL

SEQ ID NO: 172 - protein, 394 aa, synthetic construct
  REGION 1..394: note = Synthetic
MARRRRENSDKEKVTIKGRLIAGDGFTLAKLLYAMRLFRDSIEMAYRLMKKENLKFDETTKRVTKFMSNAHYAYSATKKAMAYLNQERLDLKKPQLYSIGKGCEKGNRNIRLISVDKVLIKIPHADGRHEWIEMEVKFGSRNIPIVEELIELTESDIPYSASVVLRKQKYYLYLHIPLELYAKHTAKVRVSTKAHLMAGFDINPDRVCMVIVDFNGQIRDVKTKHFPEVTLAGFPKSKAKDLRLKTLSELIDYACYHNVKCFIFERIRKIPKKKMKTRSTSRKVSKFAYAELLRHAEIMVKKRNGIFIQINPAYTSVDAIPPKQEIGIGHSHDFRIPFSDKVLKIYKHLSMPMKNLTVIRGVTSRVVFTPIGFNFNNNTSHKTIIGEFSNEKFA

SEQ ID NO: 173 - protein, 352 aa, synthetic construct
  REGION 1..352: note = Synthetic
MAHRRRQDNSKEKEHITIVGRLEFPDIVTKAKVLYTMRLFKESIERSHILLKKGLKDKEIVMRITRYMNNAHYSYSALKRAKMYRKQNKLNLRKPQLYSVGKACEKGNRNVRLISTDTVLVKVPHANGRHEWIKCKVKFGRKHIPIVSELVNPGFSYSAGIVLNDGKFYLHVTIPLEIVVRYDSHPRISEKSKYIAGFDLNSDRMNMVIINENGEILDIKNKHFPEITQPGHSKDKARDIILKSLSELIDYAVHHNVRYFVFENLNNIKRGKTGNKNANRKIARFPYRILVKHARIMVKKRNGNFATVSPAYTSIDAIPLSRKLGLDVHTTAAYILTFRYLSLHKSIYVYEK

SEQ ID NO: 174 - protein, 52 aa, synthetic construct
  REGION 1..52: note = Synthetic
MVIAMKVGESKVTSKYLVVLPKPIRIALNLMKGDYIEWHIEGDRIIVRKKNI

SEQ ID NO: 175 - protein, 350 aa, synthetic construct
  REGION 1..350: note = Synthetic
MVYRGREGNSEKKDYVTIRGRLELPDMVAKAKVFYAMRIFREAVERAFILIKKGVDDKEIARRITRYLSNAHYSYSALQRAKMYREQYRLRLKKPQLYSIGKACEKGNRNIHLTSSDEALVKIPHADGKHEWVRCSVRFGKKHIPTIRELINAGFPYSCGIVLNNGKFYIHVTVPLEIMLKHDSHPKASAKSEYIAGFDLNSDRINMVITNENGEIIDIKNKHFPEITQHGYSKNKARDTILKSLNELINYAVHHNVKCFVFEELDNIKARKTRSRNANRKISRLPYRILLTYAKTTVKKRNGTFTTVPPAYASIDAIPLSKKLGLDIHTASAYLLAFRHLESTKIYQYL

SEQ ID NO: 176 - protein, 52 aa, synthetic construct
  REGION 1..52: note = Synthetic
MVKVLKVGESKVTSKYLVVLPKAVRIALNLKRGDYLEWYIEGEKVIVRKKTM

SEQ ID NO: 177 - protein, 349 aa, synthetic construct
  REGION 1..349: note = Synthetic
MARRRWENSGEEKVTVKGRLIIEDGFALAKLLYTMRLFRDSVEMAYRLLKKEGLSLNETVRRVTRFISNAHYAYSAAKRAAAYLNQERLDLKKPQLYSIGRGCEKGNRNIRLASVDRVLIKIPHANGRHEWLEARVKFPSRYIPLVEEAIRLAENGSPYSASVVLSKGKYYLHLHIPLELYANHTAKVKVSDKARLTAGFDINPDRICMVIVDADGNIRDVKTKHFPEVTLAGFSKSKARDLRLKALSELIDYACHHNVKYFVFEKIRKISRRKMKTRSASRKVSRFAYAELLEHAKVMVEKRGGVFVQVSPAYTSVDAIPLSRKLGLDIHAASAYLLAIRRLKSISDY

SEQ ID NO: 178 - protein, 350 aa, synthetic construct
  REGION 1..350: note = Synthetic
MARGRGENRREESKEYVTIKGKIIFPDKTSEAKVLFTMRLFRDVVKKAIVFLKSSLSEAEVKRRLTKFINNAWYSSSAIKKAKTYLKQKRAKLKKPLLFSLGCKGIEKGNRNIRLIDTDKVLVKIPHANGKHEWIKCNVKFGKKYIPLIKELLTGEIAYSSTVVLSNGKYYFHVTIPLNLYAKHTGKSFVKKNASYVAGFDINPDRINMVIINEAGDIIDIRNEHFHDLIQHGYPKNKAREIRLSALSKLIDYAVNHNVKYFIFEKITNIKPDKNRKANRRIGKFAYRQLLQHAEIMVKKRDGAFKTISPAYTSIDAVPLARKFGLDRHTASAYLMAIRGLLFLKNHKNT

SEQ ID NO: 179 - protein, 49 aa, synthetic construct
  REGION 1..49: note = Synthetic
MKIGRSKIMQKSVAVIPKAVREALGLKVGDIIEWHVEEGKIVVKKAKSM

SEQ ID NO: 180 - protein, 355 aa, synthetic construct
  REGION 1..355: note = Synthetic
MARKTRTIRRAEVNYVTIKTKLEPKTSEDYLKLALLTEKFKRAVELAIRLQLKGIRKSEGVKEVSRLVLNNWWYSDSAWDYAKMLLKGAKQNGGNPLHIHLKSKFLISKPKENEKGNRNVRIEGLNVRIRSNGEWLNFKMKTGKNFLPVIFDVQKLKYGAQVVLRNGKVYLHVQVPFEVYLKHYGRTAYGKLYSGFDLNSDRVNMVILDERGIIRDVRVEHFPEVNSPGFSKRRARDLRLKALAHLLDYAFYHGVGVVFFEDLERIKRKNGKATNSRKGNRKASNFAKKELLEHGIVMALKRGFEVYLVNPAGSSKLGRELAQGLGLDVHFSSAFVIGWWGLNSLKSQKKNSPVW

SEQ ID NO: 181 - protein, 57 aa, synthetic construct
  REGION 1..57: note = Synthetic
LVNTMGVITVKIPDELEVKFRKKILDVYGVKKGALGKAITEAIELWLEKHERSVEQK

SEQ ID NO: 182 - protein, 355 aa, synthetic construct
  REGION 1..355: note = Synthetic
MARKTRRAEVNYVTIKTKLEPEKREDYLKLALLAEKFRRAVELAIRLQLKGVKKSVGVKEVSRLVLNNWWYSDSAWDYAKMLLRGAEQNGGNPRHIHLKSKFLISKPKENEKGNRNVRIEWPNVRIRSNGEWLEFRMKTAEKFLPVILDVQKFKYGAQVVLKNGKIYLHVQVLFGIYLKHLGKITRGRLYAGFDLNSDRVNMVILDETGIIRDVRVEHFPEVNSSGFSRRKARDLRLKALAQLLDYAYHHGVGTVFFEDLGKIKRRNGKAAGSRKGNRKASNFAKKELLEHGIVMASKRGFKVYLVNPADSSKLGRELARGLGLDVHTASAFVIGWWGVNSLKTHENSQKEEQFG

SEQ ID NO: 183 - protein, 54 aa, synthetic construct
  REGION 1..54: note = Synthetic
LVNTVGVITVKIPDELEVKFRKKILEVYGPRKGALGMAITEAIELWLEKHEEQK

SEQ ID NO: 184 - protein, 279 aa, synthetic construct
  REGION 1..279: note = Synthetic
VGLRQNAVERGGQNGGNPRHIHLKSKFLVSKPQENRGGNRNVRIEGMNVRIRSNGEWLNFRMSAPEKFLSVILDVQKFKYGAQVVLRNGKVYLHVQVPFGIYLKHLGKTTGGGLYAGFDLNSDRVNMVTLDENGIIRDVRVEHFPEVNSPGFSRRKARDLRLKTLARLLDYAFYHGVGTVFFEDLSRIKRRNGKATNSKKGNRKASNFAKKELLEHGVVMASKRGFDVYLVNPAGSSKLGRELAQGLGLDVHTASAFVIGWWGVNSLKTHKHPKEEQFG

SEQ ID NO: 185 - protein, 57 aa, synthetic construct
  REGION 1..57: note = Synthetic
LVNTMGVITVKIPDELEVKFRKKILDVYGVKKGALGMAITEAIELWLEKHEKSAEQK

SEQ ID NO: 186 - protein, 293 aa, synthetic construct
  REGION 1..293: note = Synthetic
MQLEGVDISTGVKELRKLVNNWWYAISAWDYANMLREGCEINKGNPRHIHVKSKFLISYAKSNEQGNRNIKVDDKIVRIRDPFSRKWLEFRHDFGGRFIKAEKYTAKVVFRNGRIYLHLSIPFELVQYGVTCGKLIASFDLNSDRINMIIVDREEKIRDVKVKHFPEVTSHGFPRNKAKDIRLKALAELLDYAYYHDVGVVLFEDLERIKRRKGRTTKTKSKKANRKISKFAKRELLHYGVTMALKRGFKVYLVNPANTSNIAKQICKKLGLDVHCTSAYVLAQKFVNSHELF

SEQ ID NO: 187 - protein, 376 aa, synthetic construct
  REGION 1..376: note = Synthetic
MSYITLTLPFNVLGDDARILIATAKQFRLVTLKVFDEFLRDEKPPLSQFSMYKGYRKVGYEILPNRRYVDGAIDLVYGAIESVNELNRLYKEMGFNGEIKLEDIEWKNWLLFQCEGESQHMNVNIKIQEDLSFRVLAFDFDGDKSYIFIRPTIPKRYELILRNLWFYMQPYYARVVLKQFGNRKGVIFTHGEVQVSIDYDFYLDFMRRFDEPKGKNVAGVDVNLDRINLAIVSRDGKLRDYKTYWFREVKSRNMPRRRVLSLIGERVHELLKYAYHHGVSRIYFEDLSVLGKLKLIWIKNGKRFNENYNYAVQTFRSRVIEMIRLKAPIYGLKTSYVNPANTSKIGEIKAKKLGVDRHTASAYIIALRGLKLFNVV

SEQ ID NO: 188 - protein, 376 aa, synthetic construct
  REGION 1..376: note = Synthetic
MDYITLILPFNISGEEARKLITTARLFRLAVLKVFNEFLSDGKPPLSQFSMYKRYRKVGYGVLPNRRYVDGAIDLVYGIIESVNELNRLHKKLGFDEEIKLEDVKFNNWLLFQCEGESKHMNINIKIREDLSFRILTFDFEGNKSYIIVKPTISKRYKPILQNIWFYMQPYYARVVLKQFGNRKGVLFTHGEIQVVVDYDFYLELMRRYNQPKGKNVAGIDVNLDRINLAIVNKDGELRDYKTFWFREVKARNMPKRRVWSIIGMRVHEMLKYAYYHGVCRIYLEDLSILGKLKLLWLKNGRRFNKNYNYAVQSFRSRVIETIKMKAPLYGMKTSYVNPANTSKISEKLARKLGVDKHTASAYVIALRGLKLLNIA

SEQ ID NO: 189 - protein, 375 aa, synthetic construct
  REGION 1..375: note = Synthetic
MNYITLTLPFNVSGDDARKLVATARLFRLVVLKAFNTFLNEGKPPLSQFSMYKRYRKVGYEILPNRRYVDGAIDLVYGVIESVNELNRLYKELKIDEEVKLEDVKFNNWLLFQCEGESKHMNINIKIREDLSFRVLTFDFEGNKSYVIVRPTIPKRWRKILQSLWFYMQPYYARVVLKQFGNRKIIFTHGKIQVAVDYDSYLEVMRKYNLPKGKNIAGIDVNLDRINLAIVNKDGELRDYKTFWFREVKARNVPRRRIWSIIGEKVHEMLEYAYHHGVNRIYLEDLSTLGKLKLLWIKNGRRFNENYNYAVQSFRSKVIEMIRMKAPLYGMVSSYVNPANTSKIGEELMRKLGVDKHTASAYVIALRGLKLLNVT

SEQ ID NO: 190 - protein, 404 aa, synthetic construct
  REGION 1..404: note = Synthetic
LVKEGLDSTYRRSIKKSLRGILGMKQRSQYVYIPEEATVRARFTVKGSEANWLLLTMARFKAGTQKALDVAKQGGTRFTDLVRASMSVIGNNRYARGSAKIVQGIIKSIKALQKLGENISLNDVELGDWWMIQSIGARNDASEKGNRNIRLVGGNTVEVLVFDGTSWRRIKAEYRASTRYNMILNEVAKLGMENKLGYLARIILLNYSHHRAYCELQVTIPWSLYVSHLPVKICVHGDNICGIDVNLDRINLAIVSKQGILLDTYTVRFPELKMQGLSRERRISTMMKAIHKALNYAVNHGCSLIVLENVKTLGYLRWAWIKRGDRRNSTWNRRVSLFTIEIAERISWYASQYGLKTYYANPSHTSKLAELIAKDMGLDRHTASAYVLALEYLGLNPRVICQNL

SEQ ID NO: 191 - protein, 322 aa, synthetic construct
  REGION 1..322: note = Synthetic
MSVIGNNRYARGSAKIVQGIVESVEVLQKLGEDISLNDVELGDWWLLQSIGSRNDASEKGNRNIRLVGGNTVEVLVYDGASWRRVRATYRASKRYAKILEEVAVLGMECRLGYLARLVLLNYSPNRAYCELQITIPWSLYERYLPTKACARGDNVCGIDVNLDRLNLAIVSKQGILLDTFTSRFPELKVQGLDRSRRTSIVMRAIHEVLNYATTHGCSLVVLENPGTLGFLKWAWIKRGERRHTSWNRRVSLYTVEVIERILWHAAQYGFRVCLVNPAYTSKLAELVARDLGLDKHTASAYILALEFLGLDPKELYQHLQRP

SEQ ID NO: 192 - protein, 408 aa, Deinococcus radiodurans
MIRNKAFVVRLYPNAAQTELINRTLGSARFVYNHFLARRIAAYKESGKGLTYGQTSSELTLLKQAEETSWLSEVDKFALQNSLKNLETAYKNFFRTVKQSGKKVGFPRFRKKRTGESYRTQFTNNNIQIGEGRLKLPKLGWVKTKGQQDIQGKILNVTVRRIHEGHYEASVLCEVEIPYLPAAPKFAAGVDVGIKDFAIVTDGVRFKHEQNPKYYRSTLKRLRKAQQTLSRRKKGSARYGKAKTKLARIHKRIVNKRQDFLHKLTTSLVREYEIIGTEHLKPDNMRKNRRLALSISDAGWGEFIRQLEYKAAWYGRLVSKVSPYFPSSQLCHDCGFKNPEVKNLAVRTWTCPNCGETHDRDENAALNIRREALVAAGISDTLNAHGGYVRPASAGNGLRSENHATLVV

SEQ ID NO: 193 - protein, 7 aa, Simian virus 40
PKKKRKV

SEQ ID NO: 194 - protein, 16 aa, synthetic construct
  REGION 1..16: note = Synthetic
KRPAATKKAGQAKKKK

SEQ ID NO: 195 - protein, 9 aa, Homo sapiens
PAAKRVKLD

SEQ ID NO: 196 - protein, 11 aa, Homo sapiens
RQRRNELKRSP

SEQ ID NO: 197 - protein, 38 aa, Homo sapiens
NQSSNFGPMKGGNFGGRSSGPYGGGGQYFAKPRNQGGY

SEQ ID NO: 198 - protein, 42 aa, Homo sapiens
RMRIZFKNKGKDTAELRRRRVEVSVELRKAKKDEQILKRRNV

SEQ ID NO: 199 - protein, 8 aa, Homo sapiens
VSRKRPRP

SEQ ID NO: 200 - protein, 8 aa, Homo sapiens
PPKKARED

SEQ ID NO: 201 - protein, 8 aa, Homo sapiens
PQPKKKPL

SEQ ID NO: 202 - protein, 12 aa, Mus sp.
SALIKKKKKMAP

SEQ ID NO: 203 - protein, 5 aa, Influenza virus
DRLRR

SEQ ID NO: 204 - protein, 7 aa, Influenza virus
PKQKKRK

SEQ ID NO: 205 - protein, 10 aa, Hepatitis D virus
RKLKKKIKKL

SEQ ID NO: 206 - protein, 10 aa, Mus sp.
REKKKFLKRR

SEQ ID NO: 207 - protein, 20 aa, Homo sapiens
KRKGDEVDGVDEVAKKKSKK

SEQ ID NO: 208 - protein, 17 aa, Homo sapiens
RKCLQAGMNLEARKTKK

SEQ ID NO: 209 - protein, 4 aa, synthetic construct
  REGION 1..4: note = Synthetic
GGGS

SEQ ID NO: 210 - protein, 5 aa, synthetic construct
  REGION 1..5: note = Synthetic
GGGGS

SEQ ID NO: 211 - protein, 9 aa, synthetic construct
  REGION 1..9: note = Synthetic
GGSGGSGGS

SEQ ID NO: 212 - protein, 15 aa, synthetic construct
  REGION 1..15: note = Synthetic
GGGGSGGGGSGGGGS

SEQ ID NO: 213 - protein, 20 aa, synthetic construct
  REGION 1..20: note = Synthetic
GGGGSGGGGSGGGGSGGGGS

SEQ ID NO: 214 - protein, 25 aa, synthetic construct
  REGION 1..25: note = Synthetic
GGGGSGGGGSGGGGSGGGGSGGGGS

SEQ ID NO: 215 - protein, 30 aa, synthetic construct
  REGION 1..30: note = Synthetic
GGGGSGGGGSGGGGSGGGGSGGGGSGGGGS

SEQ ID NO: 216 - protein, 35 aa, synthetic construct
  REGION 1..35: note = Synthetic
GGGGSGGGGSGGGGSGGGGSGGGGSGGGGSGGGGS

SEQ ID NO: 217 - protein, 40 aa, synthetic construct
  REGION 1..40: note = Synthetic
GGGGSGGGGSGGGGSGGGGSGGGGSGGGGSGGGGSGGGGS

SEQ ID NO: 218 - protein, 45 aa, synthetic construct
  REGION 1..45: note = Synthetic
GGGGSGGGGSGGGGSGGGGSGGGGSGGGGSGGGGSGGGGSGGGGS

SEQ ID NO: 219 - protein, 50 aa, synthetic construct
  REGION 1..50: note = Synthetic
GGGGSGGGGSGGGGSGGGGSGGGGSGGGGSGGGGSGGGGSGGGGSGGGGS

SEQ ID NO: 220 - protein, 55 aa, synthetic construct
  REGION 1..55: note = Synthetic
GGGGSGGGGSGGGGSGGGGSGGGGSGGGGSGGGGSGGGGSGGGGSGGGGSGGGGS

SEQ ID NO: 221 - protein, 60 aa, synthetic construct
  REGION 1..60: note = Synthetic
GGGGSGGGGSGGGGSGGGGSGGGGSGGGGSGGGGSGGGGSGGGGSGGGGSGGGGSGGGGS

SEQ ID NO: 222 - protein, 65 aa, synthetic construct
  REGION 1..65: note = Synthetic
GGGGSGGGGSGGGGSGGGGSGGGGSGGGGSGGGGSGGGGSGGGGSGGGGSGGGGSGGGGSGGGGS

SEQ ID NO: 223 - protein, 70 aa, synthetic construct
  REGION 1..70: note = Synthetic
GGGGSGGGGSGGGGSGGGGSGGGGSGGGGSGGGGSGGGGSGGGGSGGGGSGGGGSGGGGSGGGGSGGGGS

SEQ ID NO: 224 - protein, 75 aa, synthetic construct
  REGION 1..75: note = Synthetic
GGGGSGGGGSGGGGSGGGGSGGGGSGGGGSGGGGSGGGGSGGGGSGGGGSGGGGSGGGGSGGGGSGGGGSGGGGS

SEQ ID NO: 225 - protein, 10 aa, synthetic construct
  REGION 1..10: note = Synthetic
GGGGSGGGGS

SEQ ID NO: 226 - protein, 32 aa, synthetic construct
  REGION 1..32: note = Synthetic
LEPGEKPYKCPECGKSFSQSGALTRHQRTHTR

SEQ ID NO: 227 - protein, 20 aa, synthetic construct
  REGION 1..20: note = Synthetic
PKKKRKVEASSPKKRKVEAS

SEQ ID NO: 228 - DNA, 27 bp, synthetic construct
  misc_feature 1..27: note = Synthetic
  source 1..27: mol_type = other DNA
ggtggtagtggagggagcggcggttca

SEQ ID NO: 229 - DNA, 72 bp, synthetic construct
  misc_feature 1..72: note = Synthetic
  source 1..72: mol_type = other DNA
ggtggaggaggctctggtggaggcggtagcggaggcggagggtcgggtggtagtggagggagcggcggttca

SEQ ID NO: 230 - DNA, 48 bp, synthetic construct
  misc_feature 1..48: note = Synthetic
  source 1..48: mol_type = other DNA
tcgggatctgagacgcctgggacctcggaatcggctacgcccgaaagt

SEQ ID NO: 231 - DNA, 192 bp, synthetic construct
  misc_feature 1..192: note = Synthetic
  source 1..192: mol_type = other DNA
gtggataacaaatttaacaaagaaatgtgggcggcgtgggaagaaattcgtaacctgccgaacctgaacggctggcagatgaccgcgtttattgcgagcctggtggatgatccgagccagagcgcgaacctgctggcggaagcgaaaaaactgaacgatgcgcaggcgccgaaaaccggcggtggttctggt

SEQ ID NO: 232 - DNA, 108 bp, synthetic construct
  misc_feature 1..108: note = Synthetic
  source 1..108: mol_type = other DNA
ggtggttctgccggtggctccggttctggctccagcggtggcagctctggtgcgtccggcacgggtactgcgggtggcactggcagcggttccggtactggctctggc

SEQ ID NO: 233 - DNA, 12 bp, synthetic construct
  misc_feature 1..12: note = Synthetic
  source 1..12: mol_type = other DNA
acttgtttaagt

SEQ ID NO: 234 - protein, 9 aa, synthetic construct
  REGION 1..9: note = Synthetic
IEVMYPPPY

SEQ ID NO: 235 - DNA, 10 bp, synthetic construct
  misc_feature 1..10: note = Synthetic
  source 1..10: mol_type = other DNA
ccccctcccc

SEQ ID NO: 236 - RNA, 25 nt, synthetic construct
  misc_feature 1..25: note = Synthetic
  source 1..25: mol_type = other RNA
gggaacaaagctgaagtacttaccc

SEQ ID NO: 237 - DNA, 18 bp, synthetic construct
  misc_feature 1..18: note = Synthetic
  source 1..18: mol_type = other DNA
gggtagggcgggttggga

SEQ ID NO: 238 - RNA, 12 nt, synthetic construct
  misc_feature 1..12: note = Synthetic
  source 1..12: mol_type = other RNA
  misc_feature 1: note = Biotin Tag = /5Biosg/
  misc_feature 12: note = 3IAbRQSp = Iowa black quencher
tctcgtacgttc

SEQ ID NO: 239 - RNA, 24 nt, synthetic construct
  misc_feature 1..24: note = Synthetic
  source 1..24: mol_type = other RNA
  misc_feature 1: note = Biotin Tag = /5Biosg/
  misc_feature 24: note = 3IAbRQSp = Iowa black quencher
tctcgtacgttctctcgtacgttc